(19) United States
(12) Patent Application Publication
(10) Pub. No.: US 2026/0001871 A1
(43) Pub. Date: Jan. 1, 2026

(54) TRIAZOLE DERIVATIVES AS MODULATORS OF MAS-RELATED G-PROTEIN RECEPTOR D

(71) Applicant: ESCIENT PHARMACEUTICALS, INC., San Diego, CA (US)

(72) Inventors: Adam YEAGER, San Diego, CA (US); Marion LANIER, San Diego, CA (US); Brandon SELFRIDGE, San Diego, CA (US); Liming HUANG, San Diego, CA (US); Esther MARTINBOROUGH, San Diego, CA (US); Marcus BOEHM, San Diego, CA (US)

(21) Appl. No.: 18/878,466

(22) PCT Filed: Jun. 23, 2023
(86) PCT No.: PCT/US2023/026136

§ 371 Date: Dec. 23, 2024

Related U.S. Application Data
(60) Provisional application No. 63/367,012, filed on Jun. 24, 2022.

(51) Int. Cl. C07D 413/14; A61K 31/4192; A61K 31/4196; A61K 31/427; A61K 31/437; A61K 31/4439; A61K 31/444; A61K 31/506; A61K 31/519; A61K 31/5377; C07D 249/06; C07D 401/12; C07D 401/14; C07D 403/10; C07D 403/12; C07D 403/14; C07D 405/12; C07D 405/14; C07D 409/12; C07D 409/14; C07D 413/12; C07D 417/12; C07D 417/14; C07D 471/04; C07D 487/04

(52) U.S. Cl. CPC C07D 413/14; A61K 31/4192; A61K 31/4196; A61K 31/427; A61K 31/437; A61K 31/4439; A61K 31/444; A61K 31/506; A61K 31/519; A61K 31/5377; C07D 249/06; C07D 401/12; C07D 401/14; C07D 403/10; C07D 403/12; C07D 403/14; C07D 405/12; C07D 405/14; C07D 409/12; C07D 409/14; C07D 413/12; C07D 417/12; C07D 417/14; C07D 471/04; C07D 487/04

(57) ABSTRACT

Provided is a compound having the structure of Formula (I): or a pharmaceutically acceptable salt, hydrate, solvate or isotope thereof, wherein A, B, R₁, R₂, R₃, R₄, R₅, R₆, R₇, m, n, p, and y are as defined herein, for use in methods for modulating MRGPRD or for treating a MRGPRD dependent condition. Pharmaceutical compositions containing such compounds, as well as to compounds themselves, are also provided.

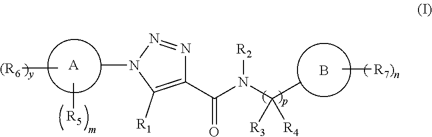

### BACKGROUND

### Technical Field

The invention relates to modulators of the Mas-related G-protein coupled receptor D, to products containing the same, as well as to methods of their use and preparation.

### Description of the Related Art

Mas-related G protein receptors (MRGPRs) are a group of orphan receptors with limited expression in very specialized tissues. Little is known about the function of most of these receptors. There are eight related receptors in this class expressed in humans, only four of which have readily identifiable orthologs in other species (i.e., MRGPR D, E, F and G). The other four receptors (MRGPR X1, X2, X3 and X4) have no counterpart, based on homology, in species other than humans and primates.

### BRIEF SUMMARY

This invention is based, in part, on the identification of MRGPRD or MRGPRD ortholog modulator compounds. MRGPRD corresponds functionally to mouse Mrgprd and rat Mrgprd. MRGPRD and its orthologs are expressed in the dorsal root ganglia as well as several peripheral organs. MRGPRD and its orthologs have been shown to be involved in pain signaling, physiological and pathophysiological processes of the gastrointestinal (GI) tract, Ca2⁺ dysregulation in the heart (cardiac output and vascular tone) and has also been shown to be expressed in skin, immune cells, the eye, kidney, and brain.

In one embodiment is provided a method of treating a MRGPRD or a MRGPRD ortholog dependent condition, by administering to a subject in need thereof an effective amount of the pharmaceutical composition of the modulator compounds of the present invention.

Accordingly, in an embodiment, methods are provided for modulating a MRGPRD by contacting the MRGPRD with an effective amount of a compound having the structure of Formula (I):

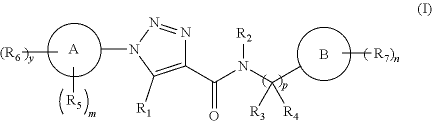

or a pharmaceutically acceptable salt, isomer, hydrate, solvate or isotope thereof, wherein A, B, R₁, R₂, R₃, R₄, R₅, R₆, R₇, m, n, p, and y are as defined below.

In other embodiments, compounds are provided having any of the structures (I′), (I″), (II), (II′), (III), (IV), (V), (VA), (VB), (VC), (VD), (VI), (VIA), (VIB), (VIC), (VID), (VII), or (VIII) as defined herein, or a pharmaceutically acceptable salt, isomer, hydrate, solvate or isotope thereof.

In some embodiments, compounds having activity as modulators of the Mas-related G-protein coupled receptor D are provided, the compounds having the following structure (IX):

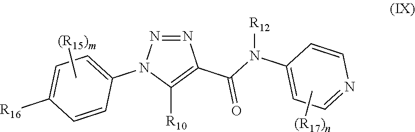

or a pharmaceutically acceptable salt, isomer, hydrate, solvate or isotope thereof, wherein R₁₀, R₁₂, R₁₅, R₁₆, R₁₇, m, and n are as defined below.

In some embodiments, compounds having activity as modulators of the Mas-related G-protein coupled receptor D are provided, the compounds having the following structure (X):

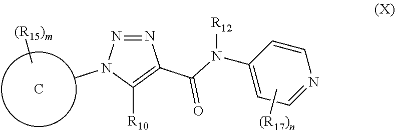

or a pharmaceutically acceptable salt, isomer, hydrate, solvate or isotope thereof, wherein C, R₁₀, R₁₂, R₁₅, R₁₇, m, and n are as defined below.

In some embodiments, compounds having activity as modulators of the Mas-related G-protein coupled receptor D are provided, the compounds having the following structure (XI):

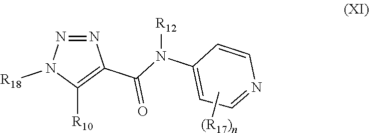

or a pharmaceutically acceptable salt, isomer, hydrate, solvate or isotope thereof, wherein R₁₀, R₁₂, R₁₇, R₁₈, and n are as defined below.

In yet other embodiments, pharmaceutical compositions are provided comprising a carrier or excipient and a compound having structure (I), or a pharmaceutically acceptable salt, isomer, hydrate, solvate or isotope thereof.

In other embodiments, pharmaceutical compositions are provided comprising structures (I′), (I″), (II), (II′), (III), (IV), (V), (VA), (VB), (VC), (VD), (VI), (VIA), (VIB), (VIC), (VID), (VII), (VIII), (IX), (X), or (XI) as defined herein or a pharmaceutically acceptable salt, isomer, hydrate, solvate or isotope thereof.

In another embodiment, methods are provided for treating an MRGPRD dependent condition by administering to a subject in need thereof an effective amount of a compound or pharmaceutical composition having structure (I), (I′), (I″), (II), (II′), (III), (IV), (V), (VA), (VB), (VC), (VD), (VI), (VIA), (VIB), (VIC), (VID), (VII), (VIII), (IX), (X), or (XI) or a pharmaceutically acceptable salt, isomer, hydrate, solvate or isotope thereof.

### DETAILED DESCRIPTION

### MRGPRs

Mas-related G-protein-coupled receptors (MRGPRs) comprise a subfamily of Class A receptors named after the first discovered member, Mas. MRGPRs were first identified on specialized sensory neurons that encode itch (pruriceptors) and pain (nociceptors). Both neuronal subtypes have their cell bodies residing in the dorsal root and trigeminal ganglia (DRG and TG). Importantly, activation of Mrgprs expressed at the surface of sensory neurons has been shown to induce both non-histaminergasrc itch, pain sensations and mechanical/visceral hypersensitivity.

The eight human MRGPRs comprise of MRGPRX1-4 and MRGPRD-G. Whilst MrgprX1-4 are only expressed in humans and non-human primates, MRGPRD-G seem to be expressed in all mammals and the orthlogs of MRGPRD are readily identified despite low sequence homology. Of note, MRGPRD is encoded by a single copy MRGPRD gene with defined orthologues in rodents and humans and thus constitutes an attractive therapeutic target for pain and other indications.

Despite the sensory nature of these receptors, the function of these MRGPRs has proved elusive. Triangulation of agonist profiles, receptor expression in tissues and loss/gain of function in animal (disease) models has proved important in recognizing the therapeutic potential of these receptors.

### MRGPRD Agonists

The first ligand described for MRGPRD of human, rat, mouse, and monkey was β-alanine. This amino acid analog is produced in the liver from uracil or from dietary carnosine by the enzyme carnosinase. Coupling to calcium-activated chloride channels via Gq proteins, phospholipase C, and inositol-3 phosphate-induced Ca2⁺ release has been described.

Alamandine, Ala1-Ang-(1-7), a peptide in the Renin-Angiotensin System (RAS), has also been described as a potent agonist of MRGPRD. Peptides seem to interact differently with the receptor as β-alanine, because β-alanine does not inhibit the vasodilatory actions of alamandine. However, D-Pro7-Ang-(1-7) was shown to be an inhibitor of alamandine binding to MRGPRD.

5-oxoETE, a lipid elevated in the gut of IBS patients, has been shown to induce calcium signaling in sensory neurons that is dependent on the presence of MRGPRD, suggesting that 5-oxoETE might signal in neurons via MRGPRD.

### MRGPRD Expression

In addition to DRG, the expression of MRGPRD transcripts has been identified in several peripheral organs including artery, heart, bladder, GI tract, eye, brain, and kidney. This suggests that MRGPRD may have a role in several peripheral indications

### MRGPRD in Neurons of the Dorsal Root Ganglia

MRGPRD is expressed at very high level in most unmyelinated nociceptive neurons that are labeled by isolectin-B4 and expressed in DRG. Like other members in the MRGPRs family, MRGPRD has been suggested to be highly related to the sensation of pain and itch.

MRGPRD activation has been shown to mediate pain signaling, characterized by hypersensitivity to multiple stimuli that lead to painful sensation once integrated in the brain. Indeed, elevated expression of MRGPRD is seen in seen in models of neuropathic pain.

### MRGPRD in the Gut

Expression of MRGPRD has been demonstrated in sensory neurons innervating the colon. In colon-projecting sensory neurons, 41% of TRPV1-positive neurons were also reported to express MRGPRD. Activation of MRGPRD signaling in the colon has been shown to participate in the development of pain sensation in the context of irritable bowel syndrome (IBS).

The expression of MRGPRD in the enteric neurons indicates that MRGPRD is highly associated with physiological and pathophysiological processes of the GI tract, such as bowel motility/dysmotility and intestinal inflammation.

In a recent study, an increase in the arachidonic acid metabolite 5-oxoETE was found in biopsies from patients with clinically established IBS compared to healthy subjects. 5-oxoETE has been shown to induce calcium signaling in sensory neurons. In the absence of MRGPRD, activation of sensory neurons by 5-oxoETE was significantly decreased, suggesting that 5-oxoETE might signal in neurons via MRGPRD.

### MRGPRD in Vascular Tissue

In the aorta/heart, MRGPRD is activated by its receptor agonist, alamandine, and produces the endothelial-dependent vasodilation in rat and mouse aortic rings. In hypertensive rats, alamandine treatment restored the contractile function and prevented Ca²⁺ dysregulation via activation of MrgprD in cardiomyocytes.

Alamandine via MRGPRD induces AMPK/NO signaling to counter-regulate ANGII-induced hypertrophy, highlighting the therapeutic potential of the alamandine/MrgD axis in the heart.

Blockers of the receptors of alternate RAS, such as the MRGPRD, increase splanchnic vascular resistance in cirrhotic animals, and thus drugs targeting the alternate RAS may be useful in the treatment of portal hypertension and liver fibrosis.

### MRGPRD in Immune Cells

MRGPRD has been found to be expressed in neutrophils and is thought to be involved in inflammatory reactions.

Alamandine, through MRGPRD receptors, do not affect MO macrophages but reduce the proinflammatory TNF-α, CCL2, and IL-1β transcript expression levels in LPS+IFN-γ-stimulated macrophages.

### MRGPRD in the Eye

MRGPRD is expressed in retinal neurons, retinal vasculature, Müller glial and RPE cells. MRGPRD-deficient mice do not exhibit gross changes in retinal morphology and thickness in aging. In vitro studies in human retinal cells show that alamandine attenuated increases in inflammatory cytokine gene expression and production of reactive oxygen species. These results support the notion that alamandine/MRGPRD may represent another new protective axis of RAS in the retina exerting anti-oxidative and anti-inflammatory effects.

### MRGPRD in the Kidney

Allantoin induces scratching behavior in mice and active DRG neurons; the calcium influx and the action potential were significantly reduced in DRG neurons of MRGPRD KO mice, suggesting a role for MRGPRD in chronic kidney disease (CKD).

### MRGPRD in the Brain

Studies in the mouse brain show that MRGPRD-positive cells have been identified in some forebrain areas, including cortex, hippocampus, amygdala, hypothalamus, habenular nuclei, striatum and pallidum, as well as in some mid-brain nuclei in a region-specific manner. The specific localization of MRGPRD in the reward- and limbic-related areas can hint at a role of MRGPRD in processes such as pain perception/modulation, synaptic plasticity, learning, memory, and cognition.

Additionally, alamandine induces antidepressant-like effects in low brain angiotensinogen transgenic rats. An MRGPRD receptor antagonist reversed the antidepressant-like effect induced by alamandine, suggesting a role of MRGPRD the treatment of neuropsychiatric diseases.

### MRGPRD and Lung Cancer

MRGPRD is reported to be expressed in lung cancer and promotes tumorigenesis. Therefore, targeting MRGPRD may provide a novel therapy for lung or other cancers.

In some embodiments, a method for the treatment of an MRGPRD dependent cancer related condition is provided, the method comprising administering an effective amount of a compound or pharmaceutical composition as described herein, or a pharmaceutically acceptable salt, ester, prodrug, solvate, hydrate or derivative thereof to a subject in need thereof. In certain embodiments, the cancer is lung cancer, pancreatic cancer, or skin cancer. In certain embodiments, the cancer is lung cancer. In other embodiments, the cancer is pancreatic cancer. In yet other embodiments, the cancer is skin cancer. In specific embodiments, the cancer is melanoma.

Embodiments of the invention also relate to a method of treating a MRGPRD dependent hyperproliferative disorder in a mammal that comprises administering to said mammal a therapeutically effective amount of a compound or pharmaceutical composition as described herein, or a pharmaceutically acceptable salt, ester, prodrug, solvate, hydrate or derivative thereof. In some embodiments, said method relates to the treatment of cancer such as acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

In certain particular embodiments, the invention relates to methods for treatment of MRGPRD dependent lung cancers, the methods comprise administering an effective amount of any of the above-described compound (or a pharmaceutical composition comprising the same) to a subject in need thereof. In certain embodiments the MRGPRD dependent lung cancer is a non-small cell lung carcinoma (NSCLC), for example adenocarcinoma, squamous-cell lung carcinoma or large-cell lung carcinoma. In other embodiments, the MRGPRD dependent lung cancer is a small cell lung carcinoma. Other MRGPRD dependent lung cancers treatable with the disclosed compounds include, but are not limited to, glandular tumors, carcinoid tumors and undifferentiated carcinomas.

Subjects that can be treated with compounds or pharmaceutical compositions of the invention, or pharmaceutically acceptable salt, isomer, hydrate, solvate or isotope of said compounds, according to the methods of this invention include, for example, subjects that have been diagnosed as having acute myeloid leukemia, acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myeloproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some embodiments subjects that are treated with the compounds or pharmaceutical compositions of the invention include subjects that have been diagnosed as having a non-cancerous hyperproliferative disorder such as benign hyperplasia of the skin (e.g., psoriasis), restenosis, or prostate (e.g., benign prostatic hypertrophy (BPH)).

### Measurement of MRGPRD Activity

See example 26.

### MRGPRD Utilities

As used herein, the phrase “MRGPRD dependent condition” means a condition where the activation, over sensitization, or desensitization of MRGPRD by a natural or synthetic ligand initiates, mediates, sustains, or augments a pathological condition.

A method of treating a subject having a pathological condition is provided, the method comprising of the administration to the subject a pharmaceutically effective amount of a compound having structure (I) or a pharmaceutically acceptable salt, isomer, hydrate, solvate or isotope thereof, or a pharmaceutical composition thereof.

As mentioned above, the invention relates to modulators of the MRGPRD, to products containing the same, as well as to methods of their use and preparation. This receptor mediates disorders including dry eye syndrome/keratoconjunctivitis sicca and related conditions, chronic itch (e.g., pruritus), inflammation disorders, autoimmunity, skin disorders, cardiovascular disease, renal disease, cognitive impairment due to neurodegenerative diseases, age-induced cognitive impairment, vascular cognitive impairment, post-stroke cognitive impairment, and psychiatric disorders.

### Definitions

As used herein, the following terms have the meaning defined below, unless the context indicates otherwise.

“Modulating” MRGPRD means that the compound interacts with the MRGPRD in a manner such that it functions as an inverse agonist to the receptor, and/or as a competitive antagonist to the receptor. In one embodiment, such modulation is partially or fully selective against other MRGPRs, such as MRGPRX1, X2, X3 and/or X4.

The term “agonism” is used herein to encompass compounds that interact in some way with a receptor and thereby function as an agonist, either by binding to the receptor at the binding site of its natural ligand or at locations other than the binding site. Thus, the phrase to “MRGPRD agonism” is used herein to encompass compounds that interact in some way with the MRGPRD and thereby function as an agonist, either by binding to the GPCR receptor at the binding site of its natural ligand, or at a location other than the binding site (i.e., allosteric binding).

Conversely, the term “antagonism” is used herein to encompass compounds that interact in some way with a receptor and thereby function as an antagonist, either by binding to the receptor at the binding site of its natural ligand or at locations other than the binding site. Thus, the phrase to “MRGPRD antagonism” is used herein to encompass compounds that interact in some way with the MRGPRD and thereby function as an antagonist, either by binding to the GPCR at the binding site of its natural ligand, or at a location other than the binding site (i.e., allosteric binding).

A partial agonist is compound that binds to and activates a receptor, but with reduced efficacy compared to a full agonist. In the presence of a full agonist, a partial agonist behaves as an effective competitive antagonist. An inverse agonist is a compound that binds to a receptor and induces an opposing pharmacological response to that of an agonist. An allosteric modulator is a compound that binds at a location distinct from the orthosteric site, or the site of action of the primary ligand, and exerts an indirect effect by influencing binding or efficacy of the primary ligand. Pure allostery exerts no effect on a protein in the absence of a primary ligand that either activates or deactivates a receptor.

“MRGPR” refers to one or more of the Mas-related G protein coupled receptors, which are a group of orphan receptors with limited expression in very specialized tissues (e.g., in mast cells and dorsal root ganglia) and barrier tissues. There are eight related receptors in this class expressed in humans, only 4 of which have readily identifiable orthologs in other species (i.e., MRGPRD, E, F and G). Some of the other four receptors (MRGPRX1, X2, X3 and X4) have counterparts in higher species including dogs and horses, but they do not have a single corresponding ortholog in rodents.

“MRGPRD,” also referred to as “MRGD,” or, “TGR7”, or “MAS related GPR family member D” refers to a member of the MRGPR family.

“Effective amount” refers to a quantity of a specified agent sufficient to achieve a desired effect in a subject being treated with that agent. Ideally, an effective amount of an agent is an amount sufficient to inhibit or treat the disease without causing substantial toxicity in the subject. The effective amount of an agent will be dependent on the subject being treated, the severity of the affliction, and the manner of administration of the pharmaceutical composition. Methods of determining an effective amount of the disclosed compound sufficient to achieve a desired effect in a subject will be understood by those of skill in the art in light of this disclosure.

In the following description, certain specific details are set forth to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this invention belongs. As used in the specification and claims, the singular form “a”, “an” and “the” include plural references unless the context clearly dictates otherwise.

“Alkyl” means a saturated or unsaturated straight chain or branched alkyl group having from 1 to 8 carbon atoms, in some embodiments from 1 to 6 carbon atoms, in some embodiments from 1 to 4 carbon atoms, and in some embodiments from 1 to 3 carbon atoms. Examples of saturated straight chain alkyl groups include, but are not limited to, methyl, ethyl, n-propyl, in-butyl, n-pentyl-, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. An unsaturated alkyl includes alkenyl and alkynyl as defined below.

“Alkenyl” means a straight chain or branched alkenyl group having from 2 to 8 carbon atoms, in some embodiments from 2 to 6 carbon atoms, in some embodiments from 2 to 4 carbon atoms, and in some embodiments from 2 to 3 carbon atoms. Alkenyl groups are unsaturated hydrocarbons that contain at least one carbon-carbon double bond. Examples of alkenyl groups include, but are not limited to, vinyl, propenyl, butenyl, pentenyl, and hexenyl.

“Alkynyl” means a straight chain or branched alkynyl group having from 2 to 8 carbon atoms, in some embodiments from 2 to 6 carbon atoms, in some embodiments from 2 to 4 carbon atoms, and in some embodiments from 2 to 3 carbon atoms. Alkynyl groups are unsaturated hydrocarbons that contain at least one carbon-carbon triple bond. Examples of alkynyl groups include, but are not limited to, ethynyl, propynyl, butynyl, pentynyl, and hexynyl.

“Halo” or “halogen” refers to fluorine, chlorine, bromine, and iodine.

“Hydroxy” or “hydroxyl” refers to —OH.

“Cyano” refers to —CN.

“Carboxy” or “carboxyl” refers to —CO₂H.

“Amino” or “aminyl” refers to —NH₂, —NHalkyl or N(alkyl)₂, wherein alkyl is as defined above. Examples of amino or aminyl include, but are not limited to —NH₂, —NHCH₃, —N(CH₃)₂, and the like.

“Aminylalkyl” refers to an amino or aminyl as described above joined by way of an alkyl as described above (i.e., -alkyl-aminyl). Examples of aminylalkyl include, but are not limited to,

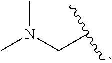 [SMILES: *CN(C)C]

and the like.

“Cyanoalkyl” refers to CN as described above joined by way of an alkyl as described above (i.e., -alkyl-CN). Examples of cyanoalkyl include, but are not limited to,

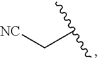 [SMILES: *CC#N]

and the like.

“Haloalkyl” refers to alkyl as defined above with one or more hydrogen atoms replaced with halogen. Examples of haloalkyl groups include, but are not limited to, —CF₃, —CHF₂, and the like.

“Hydroxylalkyl” refers to a hydroxyl as described above joined by way of an alkyl as described above i.e. -alkyl-OH). Examples of hydroxylalkyl include, but are not limited to,

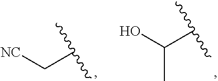

and the like.

“Alkoxy” refers to alkyl as defined above joined by way of an oxygen atom (i.e., —O-alkyl). Examples of alkoxy groups include, but are not limited to, methoxy, ethoxy, n-propoxy, n-butoxy, isopropoxy, sec-butoxy, tert-butoxy, and the like.

“Alkoxyalkyl” refers to alkoxy as described above joined by way of an alkyl as described above i.e., -alkyl-alkoxy). Examples of alkoxyalkyl groups include, but are not limited to,

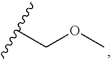 [SMILES: *COC]

and the like.

“Alkoxyalkenyl” refers to alkoxy as described above joined by way of an alkenyl as described above (i.e., -alkenyl-alkoxy). Examples of alkoxyalkenyl groups include, but are not limited to,

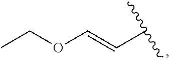 [SMILES: */C=C/OCC]

and the like.

“Haloalkoxy” refers to haloalkyl as defined above joined by way of an oxygen atom (i.e., —O-haloalkyl). Examples of haloalkoxy groups include, but are not limited to, —OCF₃, and the like.

“Alkylcarbonyl” refers to alkyl as described above joined by way of a carbonyl (i.e., —C(O)-alkyl). Examples of alkylcarbonyl groups include, but are not limited to,

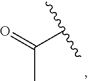 [SMILES: *C(C)=O]

and the like.

“Aminylcarbonyl” refers to aminyl as described above joined by way of a carbonyl (i.e., —C(O)-aminyl). Examples of aminylcarbonyl groups include, but are not limited to,

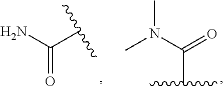

and the like.

“Alkylsulfonyl” refers to alkyl as described above joined by way of a sulfonyl (i.e., —S(O)₂-alkyl). Examples of alkylsulfonyl groups include, but are not limited to,

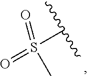 [SMILES: *S(C)(=O)=O]

and the like.

“Aminylsulfonyl” refers to aminyl as described above joined by way of a sulfonyl (i.e., —S(O)₂-aminyl). Examples of aminylsulfonyl groups include, but are not limited to,

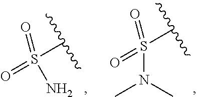

and the like.

“Alkylsulfonylaminyl” refers to alkylsulfonyl as described above joined by way of an aminyl (i.e., -aminyl-S(O)₂-alkyl). Examples of alkylsulfonylaminyl groups include, but are not limited to,

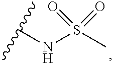 [SMILES: *NS(C)(=O)=O]

and the like.

“Cycloalkyl” refers to alkyl groups forming a ring structure, which can be substituted or unsubstituted, wherein the ring is either completely saturated, partially unsaturated, or fully unsaturated, wherein if there is unsaturation, the conjugation of the pi-electrons in the ring do not give rise to aromaticity. Examples of cycloalkyl include, but are not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. “Carbocycle” or “carbocyclyl” or “carbocyclic ring” refers to alkyl groups forming a ring structure, which can be substituted or unsubstituted, wherein the ring is either completely saturated, partially unsaturated, or fully unsaturated, wherein if there is unsaturation, the conjugation of the pi-electrons in the ring may give rise to aromaticity. In one embodiment, carbocycle includes cycloalkyl as defined above. In another embodiment, carbocycle includes aryl as defined above.

“Cycloalkylalkyl” refers to a cycloalkyl as described above joined by way of an alkyl as described above i.e. -alkyl-cycloalkyl). Examples of cycloalkylalkyl include, but are not limited to,

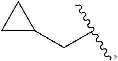 [SMILES: *CC1CC1]

and the like.

“Aryl” groups are cyclic aromatic hydrocarbons that do not contain heteroatoms. Representative aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. The terms “aryl” and “aryl groups” include fused rings wherein at least one ring, but not necessarily all rings, are aromatic, such as fused aromatic-aliphatic ring systems (e.g., indanyl, tetrahydronaphthyl, and the like). In one embodiment, aryl is phenyl or naphthyl, and in another embodiment aryl is phenyl.

“Arylalkyl” refers to an aryl group as described above jointed by way of an alkyl as described above (i.e., -alkyl-aryl). Examples of arylalkyl include, but are not limited to,

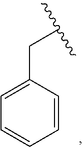 [SMILES: *Cc1ccccc1]

and the like.

“Heterocycle” or “heterocyclyl” or “heterocyclic ring” refers to aromatic and non-aromatic ring moieties containing 3 or more ring members, of which one or more is a heteroatom such as, but not limited to, N, O, S, or P. In some embodiments, heterocyclyl include 3 to 20 ring members, whereas other such groups have 3 to 15 ring members. At least one ring contains a heteroatom, but every ring in a polycyclic system need not contain a heteroatom. For example, a dioxolanyl ring and a benzdioxolanyl ring system (methylenedioxyphenyl ring system) are both heterocyclyl groups within the meaning herein.

Heterocyclyl groups also include fused ring species including those having fused aromatic and non-aromatic groups. A heterocyclyl group also includes polycyclic ring systems containing a heteroatom such as, but not limited to, quinuclidyl, and also includes heterocyclyl groups that have substituents, including but not limited to alkyl, halo, amino, aminyl, hydroxy, cyano, carboxy, nitro, thio, or alkoxy groups, bonded to one of the ring members. A heterocyclyl group as defined herein can be a heteroaryl group or a partially or completely saturated cyclic group including at least one ring heteroatom. Heterocyclyl groups include, but are not limited to, pyrrolidinyl, furanyl, tetrahydrofuranyl, dioxolanyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups.

In one embodiment, heterocyclyl includes heteroaryl.

“Heterocyclylalkyl” refers to a heterocyclyl group as described above joined by way of an alkyl as described above (i.e., -alkyl-heterocyclyl).

“Heteroaryl” refers to aromatic ring moieties containing 5 or more ring members, of which, one or more is a heteroatom such as, but not limited to, N, O, and S. Heteroaryl groups include, but are not limited to, groups such as pyrrolyl, pyrazolyl, pyridinyl, pyridazinyl, pyrimidyl, pyrazyl, pyrazinyl, pyrimidinyl, thienyl, triazolyl, tetrazolyl, triazinyl, thiazolyl, thiophenyl, oxazolyl, isoxazolyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl, and 2,3-dihydro indolyl.

“Heteroarylalkyl” refers to a heteroaryl as described above joined by way of an alkyl as described above (i.e., -alkyl-heteroaryl). Examples of heteroarylalkyl include, but are not limited to,

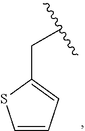 [SMILES: *Cc1cccs1]

and the like.

“Isomer” is used herein to encompass all chiral, diastereomeric or racemic forms of a structure (also referred to as a stereoisomer, as opposed to a structural or positional isomer), unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized so as to be substantially free of their enantiomeric or diastereomeric partners, and these are all within the scope of certain embodiments of the invention. The isomers resulting from the presence of a chiral center comprise a pair of nonsuperimposable-isomers that are called “enantiomers.” Single enantiomers of a pure compound are optically active (i.e., they are capable of rotating the plane of plane polarized light and designated R or S).

“Isolated optical isomer” means a compound which has been substantially purified from the corresponding optical isomer(s) of the same formula. For example, the isolated isomer may be at least about 80%, at least 80% or at least 85% pure by weight. In other embodiments, the isolated isomer is at least 90% pure or at least 98% pure, or at least 99% pure by weight.

“Substantially enantiomerically or diastereomerically” pure means a level of enantiomeric or diastereomeric enrichment of one enantiomer with respect to the other enantiomer or diastereomer of at least about 80%, and more specifically in excess of 80%, 85%, 90%, 95%, 98%, 99%, 99.5% or 99.9%.

The terms “racemate” and “racemic mixture” refer to an equal mixture of two enantiomers. A racemate is labeled “(f)” because it is not optically active (i.e., will not rotate plane-polarized light in either direction since its constituent enantiomers cancel each other out). All compounds with an asterisk (*) adjacent to a tertiary or quaternary carbon are optically active isomers, which may be purified from the respective racemate and/or synthesized by appropriate chiral synthesis.

A “hydrate” is a compound that exists in combination with water molecules. The combination can include water in stoichiometric quantities, such as a monohydrate or a dihydrate, or can include water in random amounts. As the term is used herein a “hydrate” refers to a solid form; that is, a compound in a water solution, while it may be hydrated, is not a hydrate as the term is used herein.

A “solvate” is similar to a hydrate except that a solvent other than water is present. For example, methanol or ethanol can form an “alcoholate”, which can again be stoichiometric or non-stoichiometric. As the term is used herein a “solvate” refers to a solid form; that is, a compound in a solvent solution, while it may be solvated, is not a solvate as the term is used herein.

“Isotope” refers to atoms with the same number of protons but a different number of neutrons, and an isotope of a compound of structure (1) includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes, deuterium (one proton and one neutron) and tritium (one proton and two neutrons). While fluorine has a number of isotopes, fluorine-19 is longest-lived. Thus, an isotope of a compound having the structure of structure (I) includes, but not limited to, compounds of structure (I) wherein one or more carbon 12 atoms are replaced by carbon-13 and/or carbon-14 atoms, wherein one or more hydrogen atoms are replaced with deuterium and/or tritium, and/or wherein one or more fluorine atoms are replaced by fluorine-19.

“Salt” generally refers to an organic compound, such as a carboxylic acid or an amine, in ionic form, in combination with a counter ion. For example, salts formed between acids in their anionic form and cations are referred to as “acid addition salts”. Conversely, salts formed between bases in the cationic form and anions are referred to as “base addition salts.”

The term “pharmaceutically acceptable” refers an agent that has been approved for human consumption and is generally non-toxic. For example, the term “pharmaceutically acceptable salt” refers to nontoxic inorganic or organic acid and/or base addition salts (see, e.g., Lit et al., Salt Selection for Basic Drugs, Int. J. Pharm., 33, 201-217, 1986) (incorporated by reference herein).

Pharmaceutically acceptable base addition salts of compounds of the invention include, for example, metallic salts including alkali metal, alkaline earth metal, and transition metal salts such as, for example, calcium, magnesium, potassium, sodium, and zinc salts. Pharmaceutically acceptable base addition salts also include organic salts made from basic amines such as, for example, N,N′dibenzylethylenediamine, chloroprocaine, choline, diethanolamine, ethylenediamine, meglumine (N-methylglucamine), and procaine.

Pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, aromatic aliphatic, heterocyclic, carboxylic, and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, hippuric, malonic, oxalic, embonic (pamoic), methanesulfonic, ethanesulfonic, benzenesulfonic, panthothenic, trifluoromethanesulfonic, 2-hydroxyethanesulfonic, p-toluenesulfonic, sulfanilic, cyclohexylaminosulfonic, stearic, alginic, βhydroxybutyric, salicylic, -galactaric, and galacturonic acid.

The compounds of the disclosure (i.e., compounds of structure (I) and embodiments thereof), or their pharmaceutically acceptable salts may contain one or more centers of geometric asymmetry and may thus give rise to enantiomers, diastereomers, and other stereoisomeric forms that are defined, in terms of absolute stereochemistry, as (R)- or (S)- or, as (D)- or (L)- for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also included.

Although pharmaceutically unacceptable salts are not generally useful as medicaments, such salts may be useful, for example as intermediates in the synthesis of compounds having the structure (I), for example in their purification by recrystallization.

As used herein, the phrase “MRGPRD or MRGPRD ortholog dependent condition” means a condition where the activation, over sensitization, or desensitization of MRGPRD or its ortholog by a natural or synthetic ligand initiates, mediates, sustains, or augments a pathological condition

In some embodiments, the MRGPRD dependent condition is a pain associated condition, an itch associated condition, an inflammatory condition, an ocular associated condition, a cardiovascular and renal disease associated condition, an inflammatory or autoimmune disorder, or a cognitive impairment associated condition.

As used herein, the phrase “pain associated condition” means any pain due to a medical condition. Thus, in one embodiment, the method of present invention is provided to treat a pain associated condition, such as Acute Pain, Advanced Prostate Cancer, AIDS-Related Pain, Ankylosing Spondylitis, Arachnoiditis, Arthritis, Arthrofibrosis, Ataxic Cerebral Palsy, Autoimmune Atrophic Gastritis, Avascular Necrosis, Back Pain, Behcet's Disease (Syndrome), Burning Mouth Syndrome, Bursitis, Cancer Pain, Carpal Tunnel, Cauda Equina Syndrome, Central Pain Syndrome, Cerebral Palsy, Cervical Stenosis, Charcot-Marie-Tooth (CMT) Disease, Chronic Fatigue Syndrome (CFS), Chronic Functional Abdominal Pain (CFAP), Chronic Pain, Chronic Pancreatitis, Chronic Pelvic Pain Syndrome, Collapsed Lung (Pneumothorax), Complex Regional Pain Syndrome (RSD), Constipation, Corneal Neuropathic Pain, Crohn's Disease, Degenerative Disc Disease, Dental Pain, Dercum's Disease, Dermatomyositis, Diabetic Peripheral Neuropathy (DPN), Dry Eye Syndrome, Dystonia, Ehlers-Danlos Syndrome (EDS), Endometriosis, Eosinophilia-Myalgia Syndrome (EMS), Erythromelalgia, Fibromyalgia, Gout, Headaches, Herniated disc, Hydrocephalus, Inflammatory bowel disease (IBD), Intercostal Neuraligia, Interstitial Cystitis, Irritable Bowel syndrome (IBS), Juvenile Dermatositis (Dermatomyositis), Knee Injury, Leg Pain, Loin Pain-Haematuria Syndrome, Lupus, Lyme Disease, Medullary Sponge Kidney (MSK), Meralgia Paresthetica, Mesothelioma, Migraine, Musculoskeletal pain, Myofascial Pain, Myositis, Neck Pain, Neuropathic Pain, Occipital Neuralgia, Occular Itch, Osteoarthritis, Paget's Disease, Parsonage Turner Syndrome, Pelvic Pain, Periodontitis Pain, Peripheral Neuropathy, Phantom Limb Pain, Pinched Nerve, Polycystic Kidney Disease, Polymyalgia Rhuematica, Polymyositis, Porphyria, Post Herniorraphy Pain Syndrome, Post Mastectomy, Postoperative Pain, Pain Syndrome, Post Stroke Pain, Post Thorocotomy Pain Syndrome, Postherpetic Neuralgia (Shingles), Post-Polio Syndrome, Primary Lateral Sclerosis, Psoriatic Arthritis, Pudendal Neuralgia, Radiculopathy, Raynaud's Disease, Rheumatoid Arthritis (RA), Sacroiliac Joint Dysfunction, Sarcoidosi, Scheuemann's Kyphosis Disease, Sciatica, Scoliosis, Shingles (Herpes Zoster), Sjogren's Syndrome, Spasmodic Torticollis, Sphincter of Oddi Dysfunction, Spinal Cerebellum Ataxia (SCA Ataxia), Spinal Cord Injury, Spinal Stenosis, Syringomyelia, Tarlov Cysts, Transverse Myelitis, Trigeminal Neuralgia, Neuropathic Pain, Ulcerative Colitis, Vascular Pain and Vulvodynia.

As used herein, the phrase “itch associated condition” means pruritus (including acute and chronic pruritus) associated with any condition. The itch sensation can originate, e.g., from the peripheral nervous system (e.g., dermal or neuropathic itch) or from the central nervous system (e.g., neuropathic, neurogenic or psychogenic itch). Thus, in one embodiment, the method of present invention is provided to treat an itch associated condition, such as chronic itch; contact dermatitis; Allergic blepharitis; Anaphylaxis; Anaphylactoid drug reactions; Anaphylactic shock; Anemia; Atopic dermatitis; Bullous pemphigoid; Candidiasis; Chicken pox; end-stage renal failure; hemodialysis; Cholestatic pruritis; Chronic urticaria; Contact dermatitis, Atopic Dermatitis; Dermatitis herpetiformis; Diabetes; Drug allergy, Dry skin; Dyshidrotic dermatitis; Ectopic eczema; Eosinophilic fasciitis; Epidermolysis bullosa; Erythrasma; Food allergy; Folliculitis; Fungal skin infection; Hemorrhoids; Herpes; HIV infection; Hodgkin's disease; Hyperthyroidism; Iodinated contrast dye allergy; Iron deficiency anemia; Kidney disease; Leukemia, Porphyrias; Lymphoma; Mast cell activation syndrome; Malignancy; Mastocystosis; Multiple myeloma; Neurodermatitis; Onchocerciasis; Paget's disease; Pediculosis; Polycythemia rubra vera; Prurigo nodularis; Lichen Planus; Lichen Sclerosis; Pruritus ani; Pseudo-allergic reactions; Pseudorabies; Psoriasis; Rectal prolapse; Sarcoidosis granulomas; Scabies; Schistosomiasis; Scleroderma, Severe stress, Stasia dermatitis; Swimmers itch; Thyroid disease; Tinea cruris; Uremic Pruritus; Rosacea; Cutaneous amyloidosis; Scleroderma; Acne; wound healing; burn healing, ocular itch; and Urticaria.

As used herein, the term “administration” refers to providing a compound, or a pharmaceutical composition comprising the compound as described herein. The compound or composition can be administered by another person to the subject, or it can be self-administered by the subject. Non-limiting examples of routes of administration are oral, parenteral (e.g., intravenous), or topical.

As used herein, the term “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

As used herein, the term “subject” refers to an animal (e.g., a mammal, such as a human, dog or horse). A subject to be treated according to the methods described herein may be one who has been diagnosed with a MRGPRD dependent condition or MRGPRD ortholog dependent condition, such as a pain associated condition. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition. The term “patient” may be used interchangeably with the term “subject.” A subject may refer to an adult or pediatric subject.

The Federal Food, Drug, and Cosmetic Act defines “pediatric” as a subject aged 21 or younger at the time of their diagnosis or treatment. Pediatric subpopulations are further characterized as: (i) neonates—from birth through the first 28 days of life; (ii) infants—from 29 days to less than 2 years; (iii) children—2 years to less than 12 years; and (iv) adolescents—aged 12 through 21. Despite the definition, depending on the susceptible patient population and clinical trial evaluation, an approved regulatory label may include phrasing that specifically modifies the range of a pediatric population, such as, for example, pediatric patients up to 22 years of age.

In another embodiment, the method of treating a subject having a MRGPRD dependent condition (e.g., a pain associated conditions) described herein further comprises administering to the subject a pharmaceutically effective amount of a second therapeutic agent. In another embodiment, a method of treating a subject having a pain associated condition is provided, the method comprising administering to the subject a pharmaceutically effective amount of a compound having structure (I) or a pharmaceutically acceptable salt, isomer, hydrate, solvate or isotope thereof, or a pharmaceutical composition thereof.

### Methods

### MRGPRD Modulators

In some embodiments, compounds having activity as modulators of the Mas-related G-protein coupled receptor D are provided, the compounds having the following structure (I):

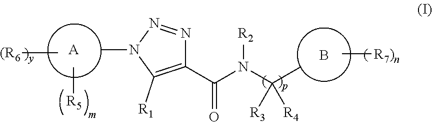

or a pharmaceutically acceptable salt, isomer, hydrate, solvate or isotope thereof, wherein:
when A is a monocyclic carbocyclic or monocyclic heterocyclic ring, then y is 1-4, or when A is a bicyclic carbocyclic or bicyclic heterocyclic ring, then y is 0-5;
B is a carbocyclic or heterocyclic ring;
R₁ is isopropyl, tert-butyl, cyclopropyl, cyclobutyl or cyclopentyl;
R₂ is H or alkyl, or R₂ joins to B to form a ring;
R₃ and R₄ are each independently H or alkyl, or R₃ and R₄ join together to form a carbocyclic ring;
R₅ is halo, OH, CN, alkyl, haloalkyl, alkoxy, haloalkoxy, alkoxyalkyl, alkoxyalkenyl, cyanoalkyl, alkylcarbonyl, aminylcarbonyl, cycloalkyl, heterocyclyl, aryl or heteroaryl;
R₆ is carbocyclyl or heterocyclyl, optionally substituted with one or more R₈;
R₇ is aminyl, halo, OH, CN, alkyl, alkenyl, alkynyl, haloalkyl, alkoxy, alkoxyalkyl, alkylcarbonyl, cycloalkyl, heterocyclyl, heteroaryl, alkylsulfonyl or alkylsulfonylaminyl;
R₈ is aminyl, halo, OH, CN, alkyl, alkenyl, alkynyl, haloalkyl, haloalkoxy, alkoxy, alkoxyalkyl, hydroxylalkyl, aminylalkyl, cyanoalkyl, carboxyl, alkylcarbonyl, aminylcarbonyl, cycloalkyl, heterocyclyl, aryl, arylalkyl, heteroaryl, heteroarylalkyl, alkylsulfonyl, aminylsulfonyl, cycloalkylalkyl, and wherein two R₈ may join together to form a carbocyclic or heterocyclic ring;
n is 0-4;
m is 0-4; and
p is 0-5.

In other embodiments are provided, compounds having the following

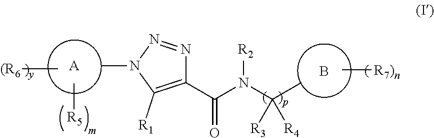

or a pharmaceutically acceptable salt, isomer, hydrate, solvate or isotope thereof, wherein:
when A is a monocyclic carbocyclic or monocyclic heterocyclic ring; and
y is 1-4.

In some embodiments, compounds are provided wherein A is a monocyclic carbocyclic ring. In other embodiments, compounds are provided wherein A is a saturated or partially saturated monocyclic carbocyclic ring. In certain embodiments, compounds are provided wherein A is phenyl. In some embodiments, compounds are provided wherein A is a monocyclic heterocyclic ring. In other embodiments, compounds are provided wherein A is a heteroaryl ring.

In some embodiments, compounds are provided wherein A is a monocyclic carbocyclic ring and y is 1. In other embodiments, compounds are provided wherein A is a monocyclic carbocyclic ring and y is 2. In additional embodiments, compounds are provided wherein A is a monocyclic carbocyclic ring and y is 3. In yet other embodiments, compounds are provided wherein A is a monocyclic carbocyclic ring and y is 4.

In some embodiments, compounds are provided wherein A is a monocyclic heterocyclic ring and y is 1. In other embodiments, compounds are provided wherein A is a monocyclic heterocyclic ring and y is 2. In additional embodiments, compounds are provided wherein A is a monocyclic heterocyclic ring and y is 3. In yet other embodiments, compounds are provided wherein A is a monocyclic heterocyclic ring and y is 4.

In some embodiments, compounds are provided wherein A is a heteroaryl ring and y is 1. In other embodiments, compounds are provided wherein A is a heteroaryl ring and y is 2. In additional embodiments, compounds are provided wherein A is a heteroaryl ring and y is 3. In yet other embodiments, compounds are provided wherein A is a heteroaryl ring and y is 4.

In some embodiments, compounds are provided having the following structure (I″):

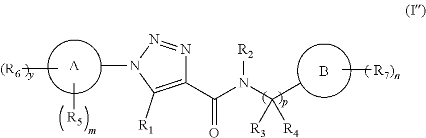

or a pharmaceutically acceptable salt, isomer, hydrate, solvate or isotope thereof, wherein:
when A is a bicyclic carbocyclic or bicyclic heterocyclic ring; and
y is 0-5.

In certain embodiments, compounds are provided wherein A is a bicyclic carbocyclic ring. In other embodiments, compounds are provided wherein A is a bicyclic heterocyclic ring. In additional embodiments, compounds are provided, wherein A is a bicyclic heteroaryl ring.

In some embodiments, compounds are provided wherein A is a bicyclic carbocyclic ring and y is 0. In other embodiments, compounds are provided wherein A is a bicyclic carbocyclic ring and y is 1. In additional embodiments, compounds are provided wherein A is a bicyclic carbocyclic ring and y is 2. In yet other embodiments, compounds are provided wherein A is a bicyclic carbocyclic ring and y is 3. In further embodiments, compounds are provided wherein A is a bicyclic carbocyclic ring and y is 4. In some embodiments, compounds are provided wherein A is a bicyclic carbocyclic ring and y is 5.

In some embodiments, compounds are provided wherein A is a bicyclic heterocyclic ring and y is 0. In other embodiments, compounds are provided wherein A is a bicyclic heterocyclic ring and y is 1. In additional embodiments, compounds are provided wherein A is a bicyclic heterocyclic ring and y is 2. In yet other embodiments, compounds are provided wherein A is a bicyclic heterocyclic ring and y is 3. In further embodiments, compounds are provided wherein A is a bicyclic heterocyclic ring and y is 4. In other embodiments, compounds are provided wherein A is a bicyclic heterocyclic ring and y is 5.

In some embodiments, compounds are provided, wherein A is a bicyclic heteroaryl ring and y is 0. In other embodiments, compounds are provided, wherein A is a bicyclic heteroaryl ring and y is 1. In additional embodiments, compounds are provided, wherein A is a bicyclic heteroaryl ring and y is 2. In yet other embodiments, compounds are provided, wherein A is a bicyclic heteroaryl ring and y is 3. In further embodiments, compounds are provided, wherein A is a bicyclic heteroaryl ring and y is 4. In some embodiments, compounds are provided, wherein A is a bicyclic heteroaryl ring and y is 5.

In other embodiments, compounds are provided, wherein A has one of the following structures:

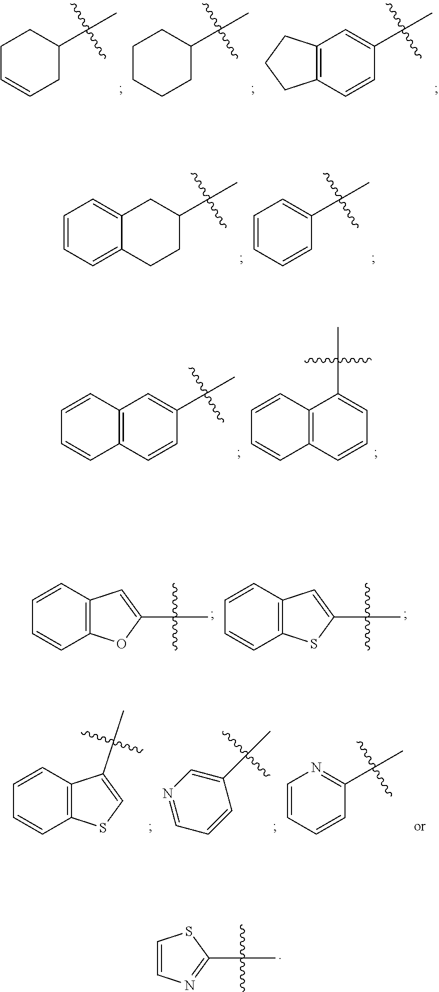

In some embodiments, compounds are provided having the following structure (II):

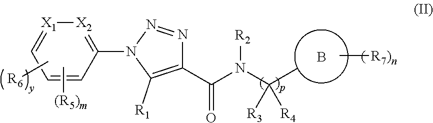

or a pharmaceutically acceptable salt, isomer, hydrate, solvate or isotope thereof, wherein:
X₁ and X₂ are both C, or X₁ is N and X2 is C, or X₁ is C and X₂ is N, and
y is 1-4.

In some embodiments, compounds are provided having the following

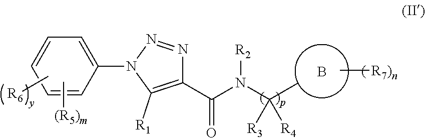

or a pharmaceutically acceptable salt, isomer, hydrate, solvate or isotope thereof, wherein:
   y is 1-4.

In some embodiments, compounds are provided having the following structure (III):

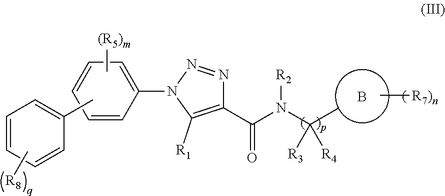

or a pharmaceutically acceptable salt, isomer, hydrate, solvate or isotope thereof, wherein:
q is 0-5.

In some embodiments, compounds are provided having the following structure (IV):

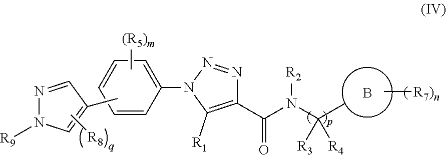

or a pharmaceutically acceptable salt, isomer, hydrate, solvate or isotope thereof, wherein:
R₉ is H, alkyl, haloalkyl, alkoxyalkyl, aminylsulfonyl, cycloalkyl, cycloalkylalkyl, heterocyclyl, heterocyclylalkyl, aryl, arylalkyl, heteroaryl, or heteroarylalkyl; and
q is 0-2.

In some embodiments, compounds are provided having the following structure (V):

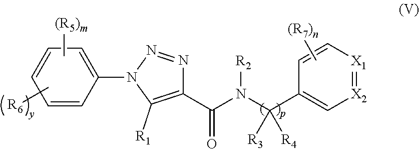

or a pharmaceutically acceptable salt, isomer, hydrate, solvate or isotope thereof, wherein:
one of X₁ and X2 is N and the other is CH optionally substituted by R₇;
n is 0 to 4; and
y is 1-4.

In some of the foregoing embodiments, compounds are provided wherein X₁ is N, X2 is CH optionally substituted by R₇, p is 0, and R₆ is a heterocyclic ring. In yet other embodiments, compounds are provided wherein X₁ is N, X2 is CH optionally substituted by R₇, p is 1, and R₆ is a heterocyclic ring. In certain embodiments, compounds are provided wherein R₆ is a 5-membered heterocyclic ring. In other certain embodiments, compounds are provided wherein R₆ is a 6-membered heterocyclic ring. In some embodiments, compounds are provided wherein R₆ is a 5-membered heteroaryl ring. In other embodiments, compounds are provided wherein R₆ is a 6-membered heteroaryl ring.

In some embodiments, compounds are provided having the following structure (VA):

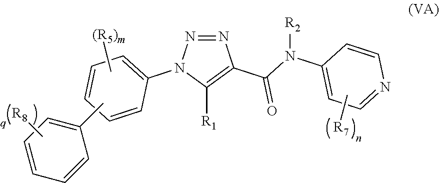

or a pharmaceutically acceptable salt, isomer, hydrate, solvate or isotope thereof, wherein:
n is 0-4; and
q is 0-5.

In some embodiments, compounds are provided having the following structure (VB):

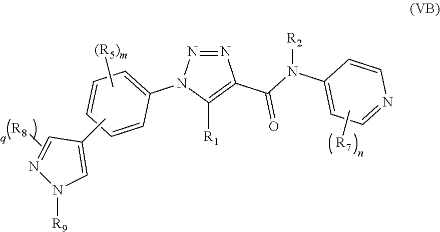

or a pharmaceutically acceptable salt, isomer, hydrate, solvate or isotope thereof, wherein:
R₉ is H, alkyl, haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl;
n is 0-4 and
q is 0-2.

In some embodiments, compounds are provided having the following structure (VC):

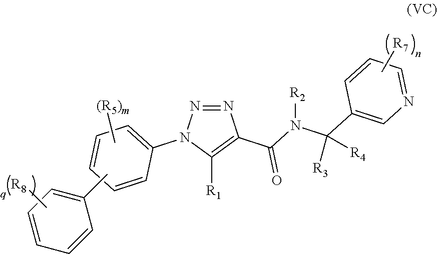

or a pharmaceutically acceptable salt, isomer, hydrate, solvate or isotope thereof, wherein:
n is 0-4; and
q is 0-5.

In some embodiments, compounds are provided having the following structure (VD):

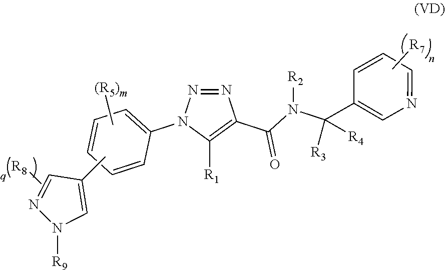

or a pharmaceutically acceptable salt, isomer, hydrate, solvate or isotope thereof, wherein:
R₉ is H, alkyl, haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl;
n is 0-4; and
q is 0-2.

In some embodiments, compounds are provided having the following structure (VI):

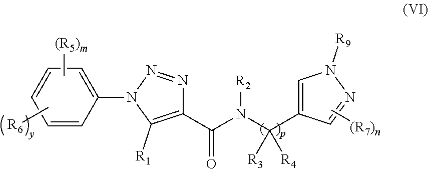

or a pharmaceutically acceptable salt, isomer, hydrate, solvate or isotope thereof, wherein:
R₉ is H, alkyl, haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl;
n is 0-2; and
y is 1-4.

In some embodiments, compounds are provided having the following structure (VIA):

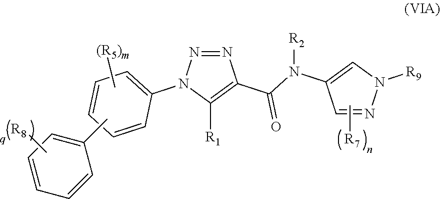

or a pharmaceutically acceptable salt, isomer, hydrate, solvate or isotope thereof, wherein:
R₉ is H, alkyl, haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl;
n is 0-4; and
q is 0-5.

In some embodiments, compounds are provided having the following structure (VIB):

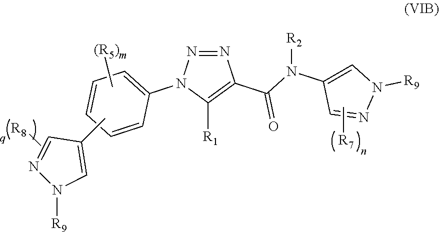

or a pharmaceutically acceptable salt, isomer, hydrate, solvate or isotope thereof, wherein:
each R₉ is independently H, alkyl, haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl;
n is 0-2; and
q is 0-2.

In some embodiments, compounds are provided having the following structure (VIC):

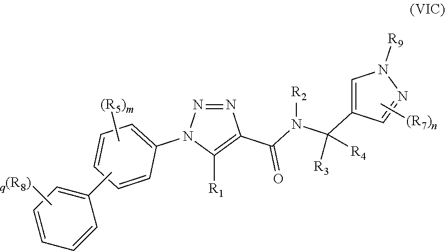

or a pharmaceutically acceptable salt, isomer, hydrate, solvate or isotope thereof, wherein
R₉ is H, alkyl, haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl;
n is 0-2; and
q is 0-5.

In some embodiments, compounds are provided having the following structure (VID):

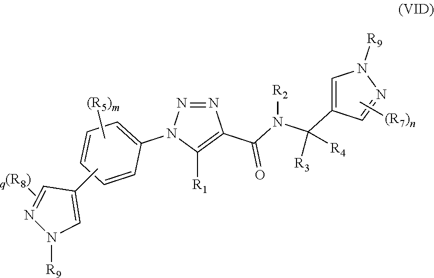

or a pharmaceutically acceptable salt, isomer, hydrate, solvate or isotope thereof, wherein:
each R₉ is independently H, alkyl, haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl;
n is 0-2; and
q is 0-2.

In some embodiments, compounds are provided having the following structure (VII):

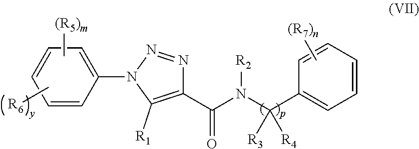

or a pharmaceutically acceptable salt, isomer, hydrate, solvate or isotope thereof, wherein:
y is 1-4.

In some embodiments, compounds are provided having the following structure (VIII):

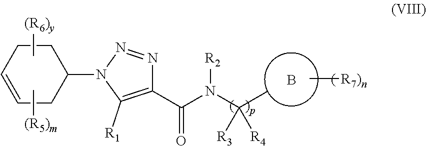

or a pharmaceutically acceptable salt, isomer, hydrate, solvate or isotope thereof, wherein:
y is 1-4.

In some embodiments, compounds are provided wherein B is a carbocyclic ring. In certain embodiments, compounds are provided wherein B is a saturated carbocyclic ring. In other embodiments, compounds are provided wherein B is an aryl ring. In some embodiments, compounds are provided wherein B is a heterocyclic ring. In some embodiments, compounds are provided wherein B is a saturated heterocyclic ring. In certain embodiments, compounds are provided wherein B is a heteroaryl ring. In some embodiments, compounds are provided wherein B has one of the following structures:

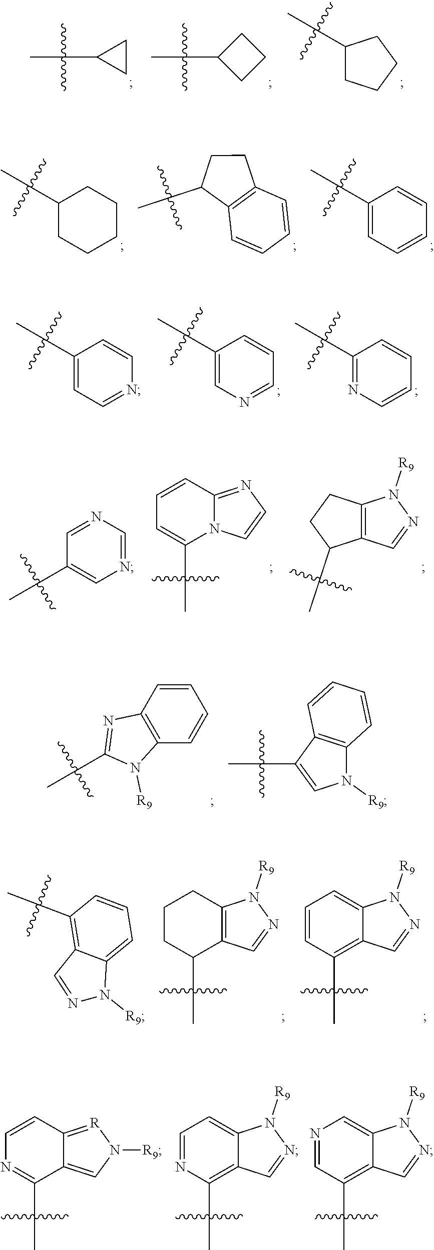

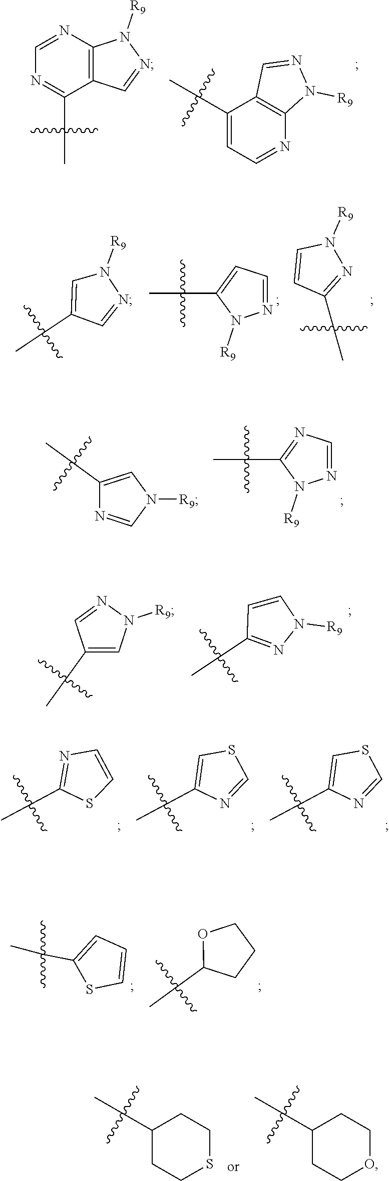

wherein B is optionally substituted by 0-4 R₇; and
wherein R₉ is H, alkyl, or haloalkyl, alkoxyalkyl, aminylsulfonyl, cycloalkyl, cycloalkylalkyl, heterocyclyl, heterocyclylalkyl, aryl, arylalkyl, heteroaryl, or heteroarylalkyl.

In some embodiments, compounds are provided wherein R₁ is isopropyl. In certain embodiments, compounds are provided wherein R₁ is tert-butyl. In other embodiments, compounds are provided wherein R₁ is cyclopropyl. In some embodiments, compounds are provided wherein R₁ is cyclobutyl. In additional embodiments, compounds are provided wherein R₁ is cyclopentyl.

In some embodiments, compounds are provided wherein R₂ is H. In other embodiments, compounds are provided wherein R₂ is alkyl. In certain embodiments, compounds are provided wherein R₂ is methyl. In additional embodiments, compounds are provided wherein R₂ is ethyl. In further embodiments, compounds are provided wherein R₂ is n-propyl. In yet other embodiments, compounds are provided wherein R₂ is isopropyl. In some embodiments, compounds are provided wherein R₂ joins to B to form a ring. In certain embodiments, compounds are provided wherein R₂ joins to B to form a ring having the following structure:

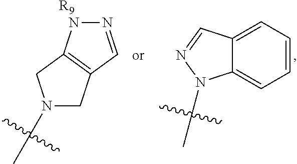

wherein the above structures are optionally substituted by 0-4 R₇; and
wherein R₉ is H, alkyl, or haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl.

In some embodiments, compounds are provided wherein R₃ and R₄ are both H. In other embodiments, compounds are provided wherein R₃ and R₄ are both alkyl. In certain embodiments, compounds are provided wherein one of R₃ and R₄ is H and the other is alkyl. In some embodiments, compounds are provided wherein the alkyl is methyl. In other embodiments, compounds are provided wherein the alkyl is ethyl. In some embodiments, compounds are provided wherein the alkyl is isopropyl. In additional embodiments, compounds are provided wherein R₃ and R₄ join together to form a carbocyclic ring. In certain embodiments, compounds are provided wherein R₃ and R₄ join together to form a 3- to 5-membered carbocyclic ring. In some embodiments, compounds are provided wherein R₃ and R₄ join together to form cyclopropyl. In other embodiments, compounds are provided wherein R₃ and R₄ join together to form cyclobutyl. In certain embodiments, compounds are provided wherein R₃ and R₄ join together to form cyclopentyl.

In some embodiments, compounds are provided wherein R₅ is halo. In certain embodiments, compounds are provided wherein R₅ is F. In other embodiments, compounds are provided wherein R₅ is Cl. In additional embodiments, compounds are provided wherein R₅ is Br. In further embodiments, compounds are provided wherein R₅ is I. In some embodiments, compounds are provided wherein R₅ is OH. In additional embodiments, compounds are provided wherein R₅ is CN. In some embodiments, compounds are provided wherein R₅ is alkyl. In certain embodiments, compounds are provided wherein R₅ is methyl. In other embodiments, compounds are provided wherein R₅ is ethyl. In further embodiments, compounds are provided wherein R₅ is n-propyl. In yet other embodiments, compounds are provided wherein R₅ is isopropyl. In some embodiments, compounds are provided wherein R₅ is tert-butyl. In some embodiments, compounds are provided wherein R₅ is haloalkyl. In certain embodiments, compounds are provided wherein R₅ is CF₃. In other embodiments, compounds are provided wherein R₅ is alkoxy. In certain embodiments, compounds are provided wherein R₅ is methoxy. In some embodiments, compounds are provided wherein R₅ is haloalkoxy. In certain embodiments, compounds are provided wherein R₅ is OCF₃. In additional embodiments, compounds are provided wherein R₅ is alkoxyalkyl. In certain embodiments, compounds are provided wherein R₅ is CH₂OCH₃. In some embodiments, compounds are provided wherein R₅ is alkoxyalkenyl. In certain embodiments, compounds are provided wherein R₅ is

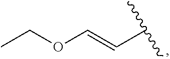 [SMILES: */C=C/OCC]

In other embodiments, compounds are provided wherein R₅ is cyanoalkyl. In certain embodiments, compounds are provided wherein R₅ is CH₂CN. In other embodiments, compounds are provided wherein R₅ is alkylcarbonyl. In certain embodiments, compounds are provided wherein R₅ is C(O)CH₃. In some embodiments, compounds are provided wherein R₅ is aminylcarbonyl. In certain embodiments, compounds are provided wherein R₅ is C(O)NH₂. In additional embodiments, compounds are provided wherein R₅ is cycloalkyl. In some embodiments, compounds are provided wherein R₅ is C₃-C₅ cycloalkyl. In certain embodiments, compounds are provided wherein R₅ is cyclopropyl. In some embodiments, compounds are provided wherein R₅ is heterocyclyl. In other embodiments, compounds are provided wherein R₅ is aryl. In additional embodiments, compounds are provided wherein R₅ is heteroaryl. In some embodiments, compounds are provided wherein R₅ has the following structures:

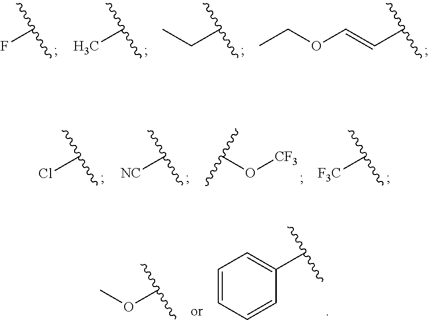

In some embodiments, compounds are provided wherein R₆ is carbocyclyl optionally substituted with one or more R₈. In other embodiments, compounds are provided wherein R₆ is heterocyclyl optionally substituted with one or more R₈. In certain embodiments, compounds are provided wherein R₆ has one of the following structures:

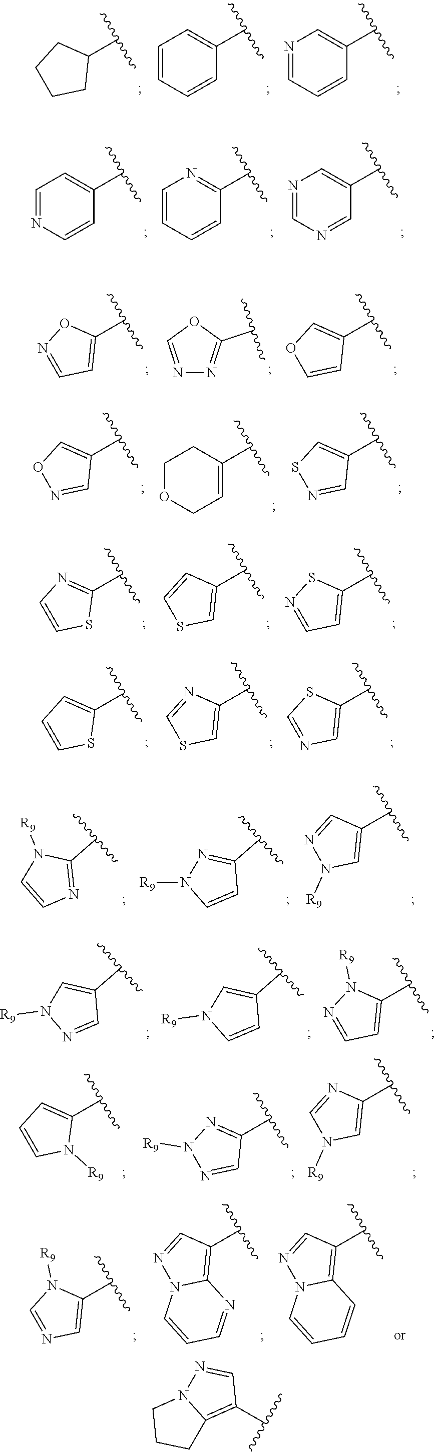

wherein R₆ is optionally substituted with one or more R₈; and
wherein R₉ is H, alkyl, or haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl.

In some embodiments, compounds are provided wherein R₇ is aminyl. In other embodiments, compounds are provided wherein R₇ is halo. In certain embodiments, compounds are provided wherein R₇ is F. In additional embodiments, compounds are provided wherein R₇ is Cl. In additional embodiments, compounds are provided wherein R₇ is Br. In further embodiments, compounds are provided wherein R₇ is I. In some embodiments, compounds are provided wherein R₇ is OH. In other embodiments, compounds are provided wherein R₇ is CN. In further embodiments, compounds are provided wherein R₇ is alkyl. In certain embodiments, compounds are provided wherein R₇ is methyl. In other embodiments, compounds are provided wherein R₇ is ethyl. In additional embodiments, compounds are provided wherein R₇ is n-propyl. In further embodiments, compounds are provided wherein R₇ is isopropyl. In yet other embodiments, compounds are provided wherein R₇ is tert-butyl. In some embodiments, compounds are provided wherein R₇ is alkenyl. In other embodiments, compounds are provided wherein R₇ is alkynyl. In certain embodiments, compounds are provided wherein R₇ is ethynyl. In some embodiments, compounds are provided wherein R₇ is haloalkyl. In certain embodiments, compounds are provided wherein R₇ is CF₃. In other embodiments, compounds are provided wherein R₇ is CHF₂. In additional embodiments, compounds are provided wherein R₇ is alkoxy. In certain embodiments, compounds are provided wherein R₇ is methoxy. In other embodiments, compounds are provided wherein R₇ is alkoxyalkyl. In certain embodiments, compounds are provided wherein R₇ is

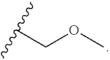 [SMILES: *COC]

In some embodiments, compounds are provided wherein R₇ is alkylcarbonyl. In certain embodiments, compounds are provided wherein R₇ is C(O)CH₃. In other embodiments, compounds are provided wherein R₇ is cycloalkyl. In certain embodiments, compounds are provided wherein R₇ is cyclopropyl. In other embodiments, compounds are provided wherein R₇ is cyclobutyl. In additional embodiments, compounds are provided wherein R₇ is cyclopentyl. In some embodiments, compounds are provided wherein R₇ is heterocyclyl. In certain embodiments, compounds are provided wherein R₇ is morpholino. In other embodiments, compounds are provided wherein R₇ is heteroaryl. In certain embodiments, compounds are provided wherein R₇ is triazolyl. In some embodiments, compounds are provided wherein R₇ is alkylsulfonyl. In certain embodiments, compounds are provided wherein R₇ is

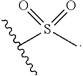 [SMILES: *S(C)(=O)=O]

In other embodiments, compounds are provided, wherein R₇ is alkylsulfonylaminyl. In certain embodiments, compounds are provided wherein R₇ is

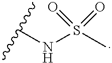 [SMILES: *NS(C)(=O)=O]

In some embodiments, compounds are provided wherein R₇ has one of the following structures:

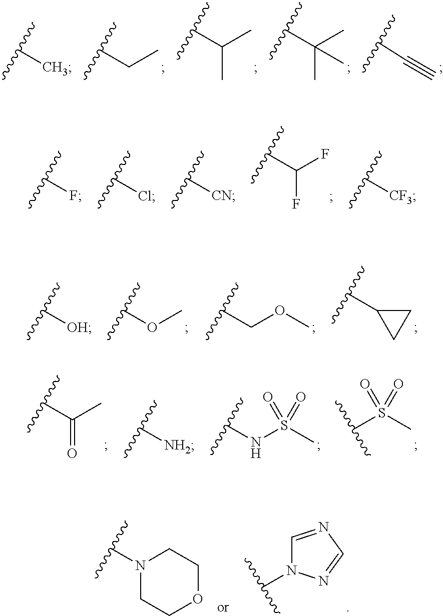

In some embodiments, compounds are provided wherein R₈ is aminyl. In certain embodiments, compounds are provided, wherein R₈ is

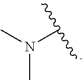 [SMILES: *N(C)C]

In other embodiments, compounds are provided wherein R₈ is halo. In certain embodiments, compounds are provided wherein R₈ is F. In additional embodiments, compounds are provided wherein R₈ is Cl. In additional embodiments, compounds are provided wherein R₈ is Br. In further embodiments, compounds are provided wherein R₈ is I. In some embodiments, compounds are provided wherein R₈ is OH. In other embodiments, compounds are provided wherein R₈ is CN. In some embodiments, compounds are provided wherein R₈ is alkyl. In certain embodiments, compounds are provided wherein R₈ is methyl. In other embodiments, compounds are provided wherein R₈ is ethyl. In additional embodiments, compounds are provided wherein R₈ is n-propyl. In yet other embodiments, compounds are provided wherein R₈ is isopropyl. In further embodiments, compounds are provided wherein R₈ is tert-butyl. In other embodiments, compounds are provided wherein R₈ is

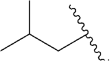 [SMILES: *CC(C)C]

In some embodiments, compounds are provided wherein R₈ is alkenyl. In other embodiments, compounds are provided wherein R₈ is alkynyl. In certain embodiments, compounds are provided wherein R₈ is ethynyl. In some embodiments, compounds are provided wherein R₈ is haloalkyl. In certain embodiments, compounds are provided wherein R₈ is CF₃. In additional embodiments, compounds are provided wherein R₈ is CHF₂. In further embodiments, compounds are provided wherein R₈ is CH₂CF₃. In some embodiments, compounds are provided wherein R₈ is haloalkoxy. In certain embodiments, compounds are provided wherein R₈ is OCF₃. In some embodiments, compounds are provided wherein R₈ is alkoxy. In certain embodiments, compounds are provided wherein R₈ is OCH₃. In additional embodiments, compounds are provided wherein R₈ is

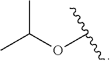 [SMILES: *OC(C)C]

In some embodiments, compounds are provided wherein R₈ is alkoxyalkyl. In certain embodiments, compounds are provided wherein R₈ is

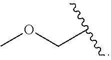 [SMILES: *COC]

In some embodiments, compounds are provided wherein R₈ is hydroxylalkyl. In certain embodiments, compounds are provided wherein R₈ is

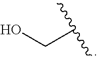 [SMILES: *CO]

In additional embodiments, compounds are provided wherein R₈ is

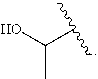 [SMILES: *C(C)O]

In other embodiments, compounds are provided wherein R₈ is aminylalkyl. In certain embodiments, compounds are provided wherein R₈ is

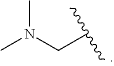 [SMILES: *CN(C)C]

In some embodiments, compounds are provided wherein R₈ is cyanoalkyl. In certain embodiments, compounds are provided wherein R₈ is

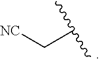 [SMILES: *CC#N]

In some embodiments, compounds are provided wherein R₈ is carboxyl. In other embodiments, compounds are provided wherein R₈ is alkylcarbonyl. In certain embodiments, compounds are provided wherein R₈ is

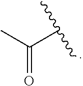 [SMILES: *C(C)=O]

In additional embodiments, compounds are provided wherein R₈ is

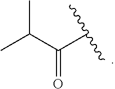 [SMILES: *C(=O)C(C)C]

In some embodiments, compounds are provided wherein R₈ is aminylcarbonyl. In certain embodiments, compounds are provided wherein R₈ is

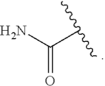 [SMILES: *C(N)=O]

In additional embodiments, compounds are provided wherein R₈ is

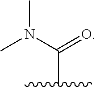 [SMILES: *C(=O)N(C)C]

In some embodiments, compounds are provided wherein R₈ is cycloalkyl. In certain embodiments, compounds are provided wherein R₈ is cyclopropyl. In additional embodiments, compounds are provided wherein R₈ is cyclobutyl. In yet other embodiments, compounds are provided wherein R₈ is cyclopentyl. In some embodiments, compounds are provided wherein R₈ is heterocyclyl. In certain embodiments, compounds are provided wherein R₈ is tetrahydropyranyl. In some embodiments, compounds are provided wherein R₈ is aryl. In certain embodiments, compounds are provided wherein R₈ is phenyl. In other embodiments, compounds are provided wherein R₈ is arylalkyl. In certain embodiments, compounds are provided wherein R₈ is

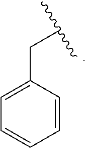 [SMILES: *Cc1ccccc1]

In some embodiments, compounds are provided wherein R₈ is heteroaryl. In other embodiments, compounds are provided wherein R₈ is heteroarylalkyl. In certain embodiments, compounds are provided wherein R₈ is

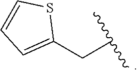 [SMILES: *Cc1cccs1]

In some embodiments, compounds are provided wherein R₈ is alkylsulfonyl. In certain embodiments, compounds are provided wherein R₈ is

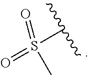 [SMILES: *S(C)(=O)=O]

In other embodiments, compounds are provided wherein R₈ is aminylsulfonyl. In certain embodiments, compounds are provided wherein R₈ is

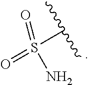 [SMILES: *S(N)(=O)=O]

In additional embodiments, compounds are provided wherein R₈ is

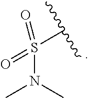 [SMILES: *S(=O)(=O)N(C)C]

In some embodiments, compounds are provided wherein R₈ is cycloalkylalkyl. In certain embodiments, compounds are provided wherein R₈ is

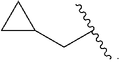 [SMILES: *CC1CC1]

In some embodiments, compounds are provided wherein two R₈ join together to form a carbocyclic ring. In other embodiments, compounds are provided wherein two R₈ join together to form a heterocyclic ring. In some embodiments, compounds are provided wherein R₈ has one of the following structures:

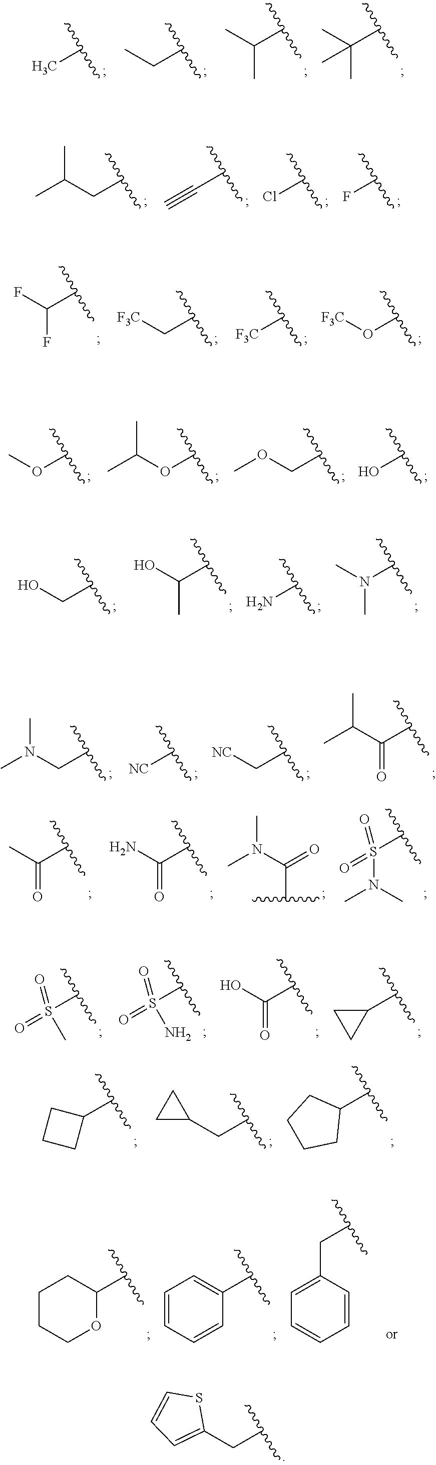

In some embodiments, compounds are provided wherein R₉ is H. In other embodiments, compounds are provided wherein R₉ is alkyl. In certain embodiments, compounds are provided wherein R₉ is methyl. In additional embodiments, compounds are provided wherein R₉ is ethyl. In further embodiments, compounds are provided wherein R₉ is n-propyl. In yet other embodiments, compounds are provided wherein R₉ is isopropyl. In some embodiments, compounds are provided wherein R₉ is tert-butyl. In additional embodiments, compounds are provided wherein R₉ is

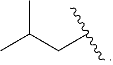 [SMILES: *CC(C)C]

In some embodiments, compounds are provided wherein R₉ is haloalkyl. In certain embodiments, compounds are provided wherein R₉ is CF₃. In additional embodiments, compounds are provided wherein R₉ is CHF₂. In yet other embodiments, compounds are provided wherein R₉ is

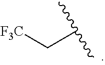 [SMILES: *CC(F)(F)F]

In some embodiments, compounds are provided wherein R₉ is alkoxyalkyl. In additional embodiments, compounds are provided wherein R₉ is

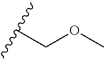 [SMILES: *COC]

In other embodiments, compounds are provided wherein R₉ is aminylsulfonyl.

In certain embodiments, compounds are provided wherein R₉ is

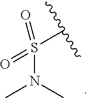 [SMILES: *S(=O)(=O)N(C)C]

In some embodiments, compounds are provided wherein R₉ is cycloalkyl. In certain embodiments, compounds are provided wherein R₉ is cyclopropyl. In additional embodiments, compounds are provided wherein R₉ is cyclobutyl. In further embodiments, compounds are provided wherein R₉ is cyclopentyl. In some embodiments, compounds are provided wherein R₉ is cycloalkylalkyl. In certain embodiments, compounds are provided wherein R₉ is

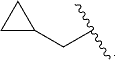 [SMILES: *CC1CC1]

In some embodiments, compounds are provided wherein R₉ is heterocyclyl. In certain embodiments, compounds are provided wherein R₉ is tetrahydropyranyl. In some embodiments, compounds are provided wherein R₉ is heterocyclylalkyl. In other embodiments, compounds are provided wherein R₉ is aryl. In certain embodiments, compounds are provided wherein R₉ is phenyl. In some embodiments, compounds are provided wherein R₉ is arylalkyl. In certain embodiments, compounds are provided wherein R₉ is

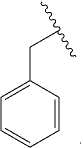 [SMILES: *Cc1ccccc1]

In some embodiments, compounds are provided wherein R₉ is heteroaryl. In other embodiments, compounds are provided wherein R₉ is heteroarylalkyl. In certain embodiments, compounds are provided wherein R₉ is

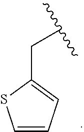 [SMILES: *Cc1cccs1]

In some embodiments, compounds are provided wherein R₉ has one of the following structures:

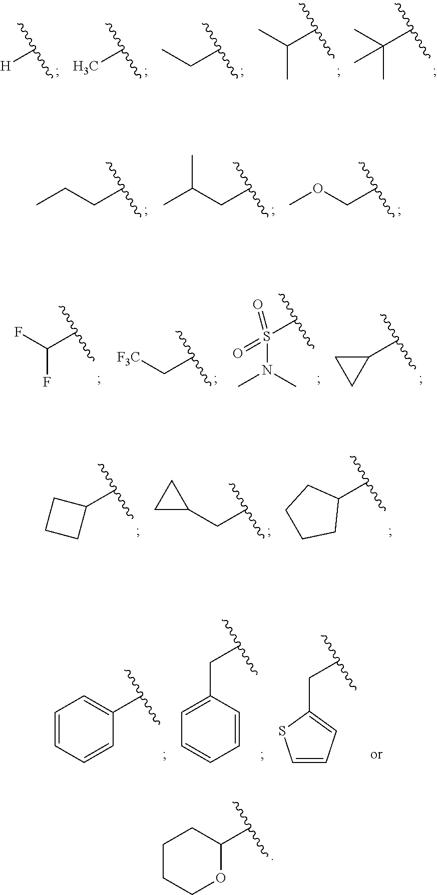

In some embodiments, compounds are provided wherein n is 0. In other embodiments, compounds are provided wherein n is 1. In additional embodiments, compounds are provided wherein n is 2. In further embodiments, compounds are provided wherein n is 3. In yet other embodiments, compounds are provided wherein n is 4.

In some embodiments, compounds are provided wherein m is 0. In other embodiments, compounds are provided wherein m is 1. In additional embodiments, compounds are provided wherein m is 2. In further embodiments, compounds are provided wherein m is 3. In yet other embodiments, compounds are provided wherein m is 4.

In some embodiments, compounds are provided wherein p is 0. In other embodiments, compounds are provided wherein p is 1. In additional embodiments, compounds are provided wherein p is 2. In further embodiments, compounds are provided wherein p is 3. In yet other embodiments, compounds are provided wherein p is 4. In some embodiments, compounds are provided wherein p is 5.

In some embodiments, compounds are provided wherein q is 0. In other embodiments, compounds are provided wherein q is 1. In additional embodiments, compounds are provided wherein q is 2.

In some embodiments, compounds having activity as modulators of the Mas-related G-protein coupled receptor D are provided, the compounds having the following structure (IX):

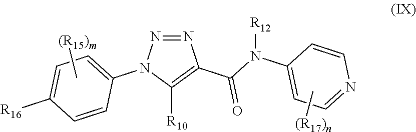

or a pharmaceutically acceptable salt, isomer, hydrate, solvate or isotope thereof, wherein:
R₁₀ is isopropyl, tert-butyl, cyclopropyl, cyclobutyl or cyclopentyl;
R₁₂ is H or alkyl;
R₁₅ is halo, alkyl, CN, OH, alkoxy, haloalkyl, cyanoalkyl or alkylcarbonyl;
R₁₆ is H, alkyl, CN, OH, alkoxy, haloalkyl, cyanoalkyl or alkylcarbonyl;
R₁₇ is halo, alkyl or haloalkyl;
m is 0-4;
n is 0-4;
provided that when R₁₆ is methyl and R₁ is cyclopropyl, then m or n is not 0; and
provided that when R₁₆ is H, R₁ is cyclopropyl, and m is 1, then R₁₅ is not F.

In other embodiments are provided, compounds having the following structure (IX):

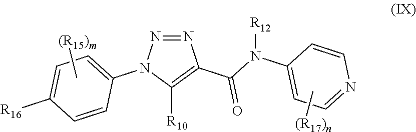

or a pharmaceutically acceptable salt, isomer, hydrate, solvate or isotope thereof, wherein:
(A) R₁₆ is H;
R₁₀ is isopropyl, tert-butyl, cyclopropyl, cyclobutyl or cyclopentyl;
R₁₂ is H or alkyl;
R₁₅ is Cl, Br, I, alkyl, CN, OH, alkoxy, haloalkyl, cyanoalkyl or alkylcarbonyl;
R₁₇ is halo, alkyl or haloalkyl;
m is 0-4; and
n is 0-4;
(B) R₁₆ is methyl and R₁₀ is cyclopropyl;
R₁₂ is H or alkyl;
R₁₅ is halo, alkyl, CN, OH, alkoxy, haloalkyl, cyanoalkyl or alkylcarbonyl;
R₁₇ is halo, alkyl or haloalkyl; and
m is 1-4 and n is 0-4, or m is 0-4 and n is 1-4;
(C) R₁₆ is methyl and R₁₀ is tert-butyl, cyclopropyl, cyclobutyl or cyclopentyl;
R₁₂ is H or alkyl;
R₁₅ is halo, alkyl, CN, OH, alkoxy, haloalkyl, cyanoalkyl or alkylcarbonyl;
R₁₇ is halo, alkyl or haloalkyl;
m is 0-4; and
n is 0-4; or
(D) R₁₆ is C₂-C₆ alkyl, CN, OH, alkoxy, haloalkyl, cyanoalkyl or alkylcarbonyl;
R₁₀ is isopropyl, tert-butyl, cyclopropyl, cyclobutyl or cyclopentyl;
R₁₂ is H or alkyl;
R₁₅ is halo, alkyl, CN, OH, alkoxy, haloalkyl, cyanoalkyl or alkylcarbonyl;
R₁₇ is halo, alkyl or haloalkyl;
m is 0-4; and
n is 0-4.

In some embodiments, compounds are provided wherein R₁₀ is isopropyl. In other embodiments, compounds are provided wherein R₁₀ is tert-butyl. In additional embodiments, compounds are provided wherein R₁₀ is cyclopropyl. In further embodiments, compounds are provided wherein R₁₀ is cyclobutyl. In yet other embodiments, compounds are provided wherein R₁₀ is cyclopentyl.

In some embodiments, compounds are provided wherein R₁₂ is H. In other embodiments, compounds are provided wherein R₁₂ is alkyl. In certain embodiments, compounds are provided wherein R₁₂ is methyl. In additional embodiments, compounds are provided wherein R₁₂ is ethyl. In further embodiments, compounds are provided wherein R₁₂ is n-propyl. In yet other embodiments, compounds are provided wherein Ru is isopropyl.

In some embodiments, compounds are provided wherein R₁₅ is halo. In certain embodiments, compounds are provided wherein R₁₅ is F. In other embodiments, compounds are provided wherein R₁₅ is Cl. In additional embodiments, compounds are provided wherein R₁₅ is Br. In further embodiments, compounds are provided wherein R₁₅ is I. In additional embodiments, compounds are provided wherein R₁₅ is Cl. In some embodiments, compounds are provided wherein R₁₅ is alkyl. In certain embodiments, compounds are provided wherein R₁₅ is methyl. In additional embodiments, compounds are provided wherein R₁₅ is ethyl. In further embodiments, compounds are provided wherein R₁₅ is n-propyl. In yet other embodiments, compounds are provided wherein R₁₅ is isopropyl. In some embodiments, compounds are provided wherein R₁₅ is tert-butyl. In some embodiments, compounds are provided wherein R₁₅ is CN. In other embodiments, compounds are provided wherein R₁₅ is OH. In some embodiments, compounds are provided wherein R₁₅ is alkoxy. In certain embodiments, compounds are provided wherein R₁₅ is methoxy. In some embodiments, compounds are provided wherein R₁₅ is haloalkyl. In certain embodiments, compounds are provided wherein R₁₅ is CF₃. In some embodiments, compounds are provided wherein R₁₅ is cyanoalkyl. In certain embodiments, compounds are provided wherein R₁₅ is CH₂CN. In other embodiments, compounds are provided wherein R₁₅ is alkylcarbonyl. In certain embodiments, compounds are provided wherein R₁₅ is C(O)CH₃.

In some embodiments, compounds are provided wherein R₁₆ is H. In other embodiments, compounds are provided wherein R₁₆ is alkyl. In certain embodiments, compounds are provided wherein R₁₆ is methyl. In additional embodiments, compounds are provided wherein R₁₆ is ethyl. In further embodiments, compounds are provided wherein R₁₆ is n-propyl. In yet other embodiments, compounds are provided wherein R₁₆ is isopropyl. In some embodiments, compounds are provided wherein R₁₆ is tert-butyl. In some embodiments, compounds are provided wherein R₁₆ is CN. In other embodiments, compounds are provided wherein R₁₆ is OH. In some embodiments, compounds are provided wherein R₁₆ is alkoxy. In certain embodiments, compounds are provided wherein R₁₆ is methoxy. In some embodiments, compounds are provided wherein R₁₆ is haloalkyl. In certain embodiments, compounds are provided wherein R₁₆ is CF₃. In some embodiments, compounds are provided wherein R₁₆ is cyanoalkyl. In certain embodiments, compounds are provided wherein R₁₆ is CH₂CN. In some embodiments, compounds are provided wherein R₁₆ is alkylcarbonyl. In certain embodiments, compounds are provided wherein R₁₆ is C(O)CH₃.

In some embodiments, compounds are provided wherein R₁₇ is halo. In certain embodiments, compounds are provided wherein R₁₇ is F. In additional embodiments, compounds are provided wherein R₁₇ is Cl. In additional embodiments, compounds are provided wherein R₁₇ is Br. In further embodiments, compounds are provided wherein R₁₇ is I. In some embodiments, compounds are provided wherein R₁₇ is alkyl. In certain embodiments, compounds are provided wherein R₁₇ is methyl. In additional embodiments, compounds are provided wherein R₁₇ is ethyl. In further embodiments, compounds are provided wherein R₁₇ is n-propyl. In yet other embodiments, compounds are provided wherein R₁₇ is isopropyl. In some embodiments, compounds are provided wherein R₁₇ is tert-butyl. In some embodiments, compounds are provided wherein R₁₇ is haloalkyl. In certain embodiments, compounds are provided wherein R₁₇ is CF₃.

In some embodiments, compounds are provided wherein m is 0. In other embodiments, compounds are provided wherein m is 1.

In some embodiments, compounds are provided wherein n is 0. In other embodiments, compounds are provided wherein n is 1. In yet other embodiments, compounds are provided wherein n is 2.

In some embodiments, compounds having activity as modulators of the Mas-related G-protein coupled receptor D are provided, the compounds having the following structure (X):

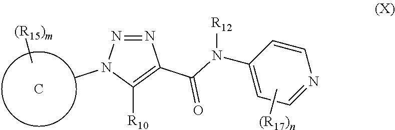

or a pharmaceutically acceptable salt, isomer, hydrate, solvate or isotope thereof, wherein:
C is a saturated carbocyclic ring;
R₁₀ is alkyl, cyclopropyl, cyclobutyl or cyclopentyl;
R₁₂ is H or alkyl;
R₁₅ is halo, alkyl, CN, OH, alkoxy, haloalkyl, cyanoalkyl or alkylcarbonyl;
R₁₇ is halo, alkyl or haloalkyl;
m is 0-4; and
n is 0-4.

In some embodiments, compounds are provided wherein C is cycloalkyl. In certain embodiments, compounds are provided wherein C is cyclopentyl. In additional embodiments, compounds are provided wherein C is cyclohexyl. In some embodiments, compounds are provided wherein R₁₀ is alkyl. In certain embodiments, compounds are provided wherein R₁₀ is methyl. In additional embodiments, compounds are provided wherein R₁₀ is ethyl. In further embodiments, compounds are provided wherein R₁₀ is n-propyl. In yet other embodiments, compounds are provided wherein R₁₀ is isopropyl. In some embodiments, compounds are provided wherein R₁₀ is tert-butyl. In some embodiments, compounds are provided wherein R₁₀ is cyclopropyl. In other embodiments, compounds are provided wherein R₁₀ is cyclobutyl. In additional embodiments, compounds are provided wherein R₁₀ is cyclopentyl.

In some embodiments, compounds are provided wherein R₁₂ is H. In other embodiments, compounds are provided wherein R₁₂ is alkyl. In certain embodiments, compounds are provided wherein R₁₂ is methyl. In additional embodiments, compounds are provided wherein R₁₂ is ethyl. In further embodiments, compounds are provided wherein R₁₂ is n-propyl. In yet other embodiments, compounds are provided wherein R₁₂ is isopropyl. In some embodiments, compounds are provided wherein R₁₂ is tert-butyl.

In some embodiments, compounds are provided wherein R₁₅ is halo. In certain embodiments, compounds are provided wherein R₁₅ is F. In additional embodiments, compounds are provided wherein R₁₅ is Cl. In additional embodiments, compounds are provided wherein R₁₅ is Br. In further embodiments, compounds are provided wherein R₁₅ is I. In some embodiments, compounds are provided wherein R₁₅ is alkyl. In certain embodiments, compounds are provided wherein R₁₅ is methyl. In additional embodiments, compounds are provided wherein R₁₅ is ethyl. In further embodiments, compounds are provided wherein R₁₅ is n-propyl. In yet other embodiments, compounds are provided wherein R₁₅ is isopropyl. In some embodiments, compounds are provided wherein R₁₅ is tert-butyl. In some embodiments, compounds are provided wherein R₁₅ is CN. In other embodiments, compounds are provided wherein R₁₅ is OH. In some embodiments, compounds are provided wherein R₁₅ is alkoxy. In certain embodiments, compounds are provided wherein R₁₅ is methoxy. In some embodiments, compounds are provided wherein R₁₅ is haloalkyl. In certain embodiments, compounds are provided wherein R₁₅ is CF₃. In some embodiments, compounds are provided wherein R₁₅ is cyanoalkyl. In certain embodiments, compounds are provided wherein R₁₅ is CH₂CN. In some embodiments, compounds are provided wherein R₁₅ is alkylcarbonyl. In certain embodiments, compounds are provided wherein R₁₅ is C(O)CH₃.

In some embodiments, compounds are provided wherein R₁₇ is halo. In certain embodiments, compounds are provided wherein R₁₇ is F. In additional embodiments, compounds are provided wherein R₁₇ is Cl. In additional embodiments, compounds are provided wherein R₁₇ is Br. In further embodiments, compounds are provided wherein R₁₇ is I. In some embodiments, compounds are provided wherein R₁₇ is alkyl. In certain embodiments, compounds are provided wherein R₁₇ is methyl. In additional embodiments, compounds are provided wherein R₁₇ is ethyl. In further embodiments, compounds are provided wherein R₁₇ is n-propyl. In yet other embodiments, compounds are provided wherein R₁₇ is isopropyl. In some embodiments, compounds are provided wherein R₁₇ is tert-butyl. In some embodiments, compounds are provided wherein R₁₇ is haloalkyl. In certain embodiments, compounds are provided wherein R₁₇ is CF₃.

In some embodiments, compounds are provided wherein m is 0. In other embodiments, compounds are provided wherein m is 1.

In some embodiments, compounds are provided wherein n is 0. In other embodiments, compounds are provided wherein n is 1. In yet other embodiments, compounds are provided wherein n is 2.

In some embodiments, compounds having activity as modulators of the Mas-related G-protein coupled receptor D are provided, the compounds having the following structure (XI):

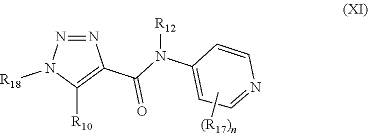

or a pharmaceutically acceptable salt, isomer, hydrate, solvate or isotope thereof, wherein:
R₁₀ is isopropyl, tert-butyl, cyclopropyl, cyclobutyl or cyclopentyl;
R₁₂ is H or alkyl;
R₁₇ is halo, alkyl or haloalkyl;
R₁₈ is an alkyl group optionally substituted with one or more R₁₉;
R₁₉ is halo, CN, OH, alkoxy, haloalkyl, or alkylcarbonyl; and
n is 0-4.

In some embodiments, compounds are provided wherein R₁₀ is isopropyl. In other embodiments, compounds are provided wherein R₁₀ is tert-butyl. In additional embodiments, compounds are provided wherein R₁₀ is cyclopropyl. In further embodiments, compounds are provided wherein R₁₀ is cyclobutyl. In yet other embodiments, compounds are provided wherein R₁₀ is cyclopentyl.

In some embodiments, compounds are provided wherein R₁₂ is H. In other embodiments, compounds are provided wherein R₁₂ is alkyl. In certain embodiments, compounds are provided wherein R₁₂ is methyl. In additional embodiments, compounds are provided wherein R₁₂ is ethyl. In further embodiments, compounds are provided wherein R₁₇ is n-propyl. In yet other embodiments, compounds are provided wherein RI is isopropyl. In some embodiments, compounds are provided wherein R₁₂ is tert-butyl.

In some embodiments, compounds are provided wherein R₁₇ is halo. In certain embodiments, compounds are provided wherein R₁₇ is F. In additional embodiments, compounds are provided wherein R₁₇ is Cl. In additional embodiments, compounds are provided wherein R₁₇ is Br. In further embodiments, compounds are provided wherein R₁₇ is I. In some embodiments, compounds are provided wherein R₁₇ is alkyl. In certain embodiments, compounds are provided wherein R₁₇ is methyl. In additional embodiments, compounds are provided wherein R₁₇ is ethyl. In further embodiments, compounds are provided wherein R₁₇ is n-propyl. In yet other embodiments, compounds are provided wherein R₁₇ is isopropyl. In some embodiments, compounds are provided wherein R₁₇ is tert-butyl. In some embodiments, compounds are provided wherein R₁₇ is haloalkyl. In certain embodiments, compounds are provided wherein R₁₇ is CF₃.

In some embodiments, compounds are provided wherein R₁₈ is C₁-C₄ alkyl. In some embodiments, compounds are provided wherein R₁₈ is C₁-C₄ alkyl substituted with one R₁₉. In some embodiments, compounds are provided wherein R₁₈ is methyl substituted with one R₁₉. In some embodiments, compounds are provided wherein R₁₈ is ethyl substituted with one R₁₉.

In some embodiments, compounds are provided wherein R₁₉ is halo. In certain embodiments, compounds are provided wherein R₁₉ is F. In additional embodiments, compounds are provided wherein R₁₉ is Cl. In additional embodiments, compounds are provided wherein R₁₉ is Br. In further embodiments, compounds are provided wherein R₁₉ is I. In some embodiments, compounds are provided wherein R₁₉ is CN. In other embodiments, compounds are provided wherein R₁₉ is OH. In some embodiments, compounds are provided wherein R₁₉ is haloalkyl. In certain embodiments, compounds are provided wherein R₁₉ is CF₃. In some embodiments, compounds are provided wherein R₁₉ is alkoxy. In certain embodiments, compounds are provided wherein R₁₉ is methoxy. In some embodiments, compounds are provided wherein R₁₉ is alkylcarbonyl. In certain embodiments, compounds are provided wherein R₁₉ is C(O)CH₃.

In some embodiments, compounds are provided wherein n is 0. In other embodiments, compounds are provided wherein n is 1. In yet other embodiments, compounds are provided wherein n is 2.

See, Table A for exemplary compound structures according to the above.

### Indications

### Pain

In one embodiment, the malcondition for which modulation of MRGPRD is medically indicated, the pain associated condition is: Acute Pain, Advanced Prostate Cancer, AIDS-Related Pain, Ankylosing Spondylitis, Arachnoiditis, Arthritis, Arthrofibrosis, Ataxic Cerebral Palsy, Autoimmune Atrophic Gastritis, Avascular Necrosis, Back Pain, Behcet's Disease (Syndrome), Burning Mouth Syndrome, Bursitis, Cancer Pain, Carpal Tunnel, Cauda Equina Syndrome, Central Pain Syndrome, Cerebral Palsy, Cervical Stenosis, Charcot-Marie-Tooth (CMT) Disease, Chronic Fatigue Syndrome (CFS), Chronic Functional Abdominal Pain (CFAP), Chronic Pain, Chronic Pancreatitis, Chronic Pelvic Pain Syndrome, Collapsed Lung (Pneumothorax), Complex Regional Pain Syndrome (RSD), Constipation, Corneal Neuropathic Pain, Crohn's Disease, Degenerative Disc Disease, Dental Pain, Dercum's Disease, Dermatomyositis, Diabetic Peripheral Neuropathy (DPN), Dry Eye Syndrome, Dystonia, Ehlers-Danlos Syndrome (EDS), Endometriosis, Eosinophilia-Myalgia Syndrome (EMS), Erythromelalgia, Fibromyalgia, Gout, Headaches, Herniated disc, Hydrocephalus, Inflammatory Bowel Disease (IBD), Intercostal Neuraligia, Interstitial Cystitis, Irritable Bowel syndrome (IBS), Juvenile Dermatositis (Dermatomyositis), Knee Injury, Leg Pain, Loin Pain-Haematuria Syndrome, Lupus, Lyme Disease, Medullary Sponge Kidney (MSK), Meralgia Paresthetica, Mesothelioma, Migraine, Musculoskeletal pain, Myofascial Pain, Myositis, Neck Pain, Neuropathic Pain, Occipital Neuralgia, ocular Itch, Osteoarthritis, Paget's Disease, Parsonage Turner Syndrome, Pelvic Pain, Periodontitis Pain, Peripheral Neuropathy, Phantom Limb Pain, Pinched Nerve, Polycystic Kidney Disease, Polymyalgia Rhuematica, Polymyositis, Porphyria, Post Herniorraphy Pain Syndrome, Post Mastectomy, Postoperative Pain, Pain Syndrome, Post Stroke Pain, Post Thorocotomy Pain Syndrome, Postherpetic Neuralgia (Shingles), Post-Polio Syndrome, Primary Lateral Sclerosis, Psoriatic Arthritis, Pudendal Neuralgia, Radiculopathy, Raynaud's Disease, Rheumatoid Arthritis (RA), Sacroiliac Joint Dysfunction, Sarcoidosi, Scheuemann's Kyphosis Disease, Sciatica, Scoliosis, Shingles (Herpes Zoster), Sjogren's Syndrome, Spasmodic Torticollis, Sphincter of Oddi Dysfunction, Spinal Cerebellum Ataxia (SCA Ataxia), Spinal Cord Injury, Spinal Stenosis, Syringomyelia, Tarlov Cysts, Transverse Myelitis, Trigeminal Neuralgia, Neuropathic Pain, Ulcerative Colitis, Vascular Pain or Vulvodynia.

### Skin

In another embodiment, the malcondition for which modulation of MRGPRD is medically indicated, the itch associated condition is: chronic itch; contact dermatitis; Allergic blepharitis; Anemia; Atopic dermatitis; Bullous pemphigoid; Candidiasis; Chicken pox; end-stage renal failure; hemodialysis; Chronic urticaria; Contact dermatitis, Atopic Dermatitis; Dermatitis herpetiformis; Diabetes; Drug allergy, Dry skin; Dyshidrotic dermatitis; Ectopic eczema; Eosinophilic fasciitis; Epidermolysis bullosa; Erythrasma; Food allergy; Folliculitis; Fungal skin infection; Hemorrhoids; Herpes; HIV infection; Hodgkin's disease; Hyperthyroidism; Iodinated contrast dye allergy; Iron deficiency anemia; Kidney disease; Leukemia, porphyrias; Lymphoma; Malignancy; Mastocystosis, Multiple myeloma; Neurodermatitis; Onchocerciasis; Paget's disease; Pediculosis; Polycythemia rubra vera; Prurigo nodularis; Lichen Planus; Lichen Sclerosis; Pruritus ani; Pseudorabies; Psoriasis; Rectal prolapse; Sarcoidosis granulomas; Scabies; Schistosomiasis; Scleroderma, Severe stress, Stasia dermatitis; Swimmer's itch; Thyroid disease; Tinea cruris, Rosacea; Cutaneous amyloidosis; Scleroderma; Acne; wound healing; burn healing; ocular itch; or Urticaria.

In a specific embodiment, the itch associated condition is urticaria, pruritus, atopic dermatitis, dry skin, psoriasis, contact dermatitis, or eczema.

### Ocular

In another embodiment, the malcondition for which modulation of MRGPRD is medically indicated, the ocular associated condition is: dry eye syndrome/keratoconjunctivitis sicca and related conditions, including xeropthalmia, meibomian gland dysfunction and lacrimal gland dysfunction; dry eye associated with other medical conditions including dacryoadenitis, dacryocystitis, allergic conjunctivitis, blepharitis, rheumatoid arthritis, systemic lupus erythematous, scleroderma, Sjogren's syndrome, Stevens-Johnson syndrome, sarcoidosis, sympathetic opthalmia, diabetic retinopathy, parasitic eye infections, thyroid disorders, and vitamin A deficiency; dry eye associated with medications such as antihistamines, decongestants, anti-depressants, tranquilizers, diuretics, hormone replacement, oral contraceptives, antihypertensives, isotretonin treatments for acne, and anticholinergic drugs; and dry eye associated with eye surgery including laser eye surgery, glaucoma surgery, corneal transplantation, and cataract removal surgery.

### Cardiovascular and Renal

In another embodiment, the malcondition for which modulation of MRGPRD is medically indicated, cardiovascular and renal diseases associated condition is: peripheral vascular disease, cerebrovascular disease, coronary artery disease, cardiac hypertrophy, cardiac fibrosis, cardiovascular hypertension, renovascular hypertension, renal fibrosis, renal disease, nephritis, atherosclerosis, coronary atherosclerotic heart disease, acute myocardial infarction, stroke, thrombosis, coronary atherothrombosis, pulmonary embolism, myocardial ischemia, carotid stenosis, vertebral stenosis, intracranial stenosis, and aneurysms as well as treatment of cardiac dysfunction induced by sepsis, rheumatic fever, or other acute or chronic disorders that influence cardiovascular and renal function such as diabetes.

### Immune/GI

In another embodiment, the malcondition for which modulation of MRGPRD is medically indicated, the chronic inflammatory and autoimmune associated condition include: chronic pulmonary allergy, asthma, chronic bronchitis, atherosclerosis, Graves' disease, Hashimoto's thyroiditis, chronic inflammatory demyelinating polyneuropathy, ankylosing spondylitis, sacroiliitis, steatohepatitis, scleroderma, systemic sclerosis, diabetes, ulcerative colitis, Crohn's disease, inflammatory bowel disease, systemic lupus erythematous, alopecia areata, temporal arteritis, chronic peptic ulcer, polymyalgia rheumatica, periodontitis, sinusitis, rhinitis, pancreatitis, nephritis, Sjogren's syndrome, dermatomyositis, polymyositis, inclusion body myositis, autoimmune necrotizing myopathy, idiopathic inflammatory myopathies, multiple sclerosis, rheumatoid arthritis, and vasculitis.

As used herein, the term “autoimmune disorder”, or “inflammatory disorder” means a disease or disorder arising from and/or directed against an individual's own tissues or organs, or a co-segregate or manifestation thereof, or resulting condition therefrom. Typically, various clinical and laboratory markers of autoimmune diseases may exist including, but not limited to, hypergammaglobulinemia, high levels of autoantibodies, antigen-antibody complex deposits in tissues, clinical benefit from corticosteroid or immunosuppressive treatments, and lymphoid cell aggregates in affected tissues. Thus, in one embodiment, the method of present invention is provided to treat an autoimmune disorder, such as chronic inflammation, mast cell activation syndrome, Multiple Sclerosis, Steven Johnson's Syndrome, Toxic Epidermal Necrolysis, appendicitis, bursitis, cutaneous lupus, colitis, cystitis, dermatitis, phlebitis, reflex sympathetic dystrophy/complex regional pain syndrome (rsd/crps), rhinitis, tendonitis, tonsillitis, acne vulgaris, sinusitis, rosacea, psoriasis, graft-versus-host disease, reactive airway disorder, asthma, airway infection, allergic rhinitis, autoinflammatory disease, celiac disease, chronic prostatitis, diverticulitis, glomerulonephritis, hidradenitis suppurativa, hypersensitivities, intestinal disorder, epithelial intestinal disorder, inflammatory bowel disease, irritable bowel syndrome, Crohn's Disease, ulcerative colitis, lupus erythematous, interstitial cystitis, otitis, pelvic inflammatory disease, endometrial pain, reperfusion injury, rheumatic fever, rheumatoid arthritis, sarcoidosis, transplant rejection, psoriasis, lung inflammation, chronic obstructive pulmonary disease, permanent sputum eosinophilia, eosinophilic leukemia, eosinophilic esophagitis, eosinophilic gastritis, mast cell gastrointestinal disease, hypereosinophilic syndrome, aspirin-exacerbated respiratory disease, nasal polyposis, chronic rhinosinusitis, antibody-dependent cell-mediated cytotoxicity, neurofibromatosis, swannamatoisis, tubulointerstitial nephritis, glomerulonephritis, diabetic nephropathy, allograft rejection, amyloidosis, renovascular ischemia, reflux nephropathy, polycystic kidney disease, liver fibrosis/cirrhosis, autoimmune liver disease, Biliary atresia, acute and chronic Hepatitis B and C virus, Liver tumors and cancer, Lung tumors and cancer, Alcoholic liver disease, Polycystic liver disease, Liver cholangiocarcinoma, neuromyelitis optica spectum disorder, cardiovascular disease, and vasculitis.

### Cognitive Indications

In another embodiment, the malcondition for which modulation of MRGPRD is medically indicated, cognitive impairment associated condition include neurodegenerative diseases including Parkinson's disease, Alzheimer's disease, Huntington's disease, Lewy body dementia, frontotemporal dementia, progressive supranuclear palsy, corticobasal syndrome, frontotemporal lobar degeneration, amyotrophic lateral sclerosis and multiple sclerosis, as well as age-induced cognitive impairment, vascular cognitive impairment and post-stroke cognitive impairment.

### Cancer

In another embodiment, the malcondition for which modulation of MRGPRD is medically indicated, the cancer related condition is: acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

In another embodiment, the cancer related condition is lung cancer, pancreatic cancer, or skin cancer. In a specific embodiment, the cancer related condition is lung cancer. In another specific embodiment the cancer related condition is pancreatic cancer. In another specific embodiment, the cancer related condition is skin cancer. In a certain embodiment the skin cancer related condition is melanoma.

In one embodiment, a compound is selected from any one of the compounds listed in Table A, or a pharmaceutically acceptable salt, isomer, hydrate, solvate or isotope thereof.

Representative compounds of Formula (I), as well as Formulas (II) through (XI) as applicable, include any one of the compounds listed in Table A below, as well as a pharmaceutically acceptable isomer, racemate, hydrate, solvate, isotope, or salt thereof. To this end, representative compounds are identified herein by their respective “Compound Number”, which is sometimes abbreviated as “Compound No.” or “Cpd. No.”

TABLE A
Representative Compounds

| Cpd. | |
|---|---|
| No. | Structure |
| 1-4 | 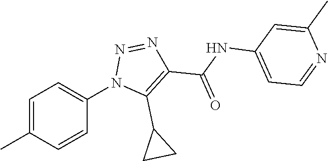 |
| 1-5 | 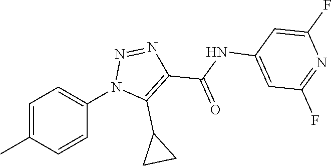 [SMILES: Cc1ccc(-n2nnc(C(=O)Nc3cc(F)nc(F)c3)c2C2CC2)cc1] |
| 1-6 | 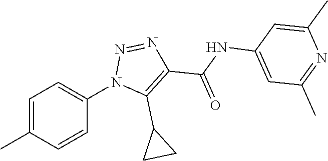 |
| 1-7 | 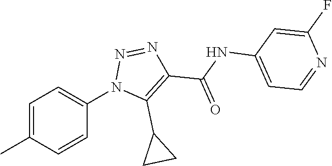 [SMILES: Cc1ccc(-n2nnc(C(=O)Nc3ccnc(F)c3)c2C2CC2)cc1] |
| 1-9 | 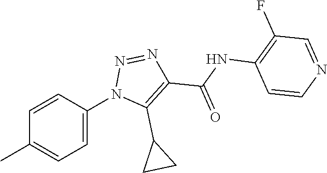 [SMILES: Cc1ccc(-n2nnc(C(=O)Nc3ccncc3F)c2C2CC2)cc1] |
| 1-19 | 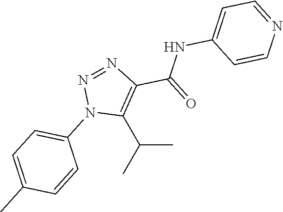 [SMILES: Cc1ccc(-n2nnc(C(=O)Nc3ccncc3)c2C(C)C)cc1] |
| 2-1 | 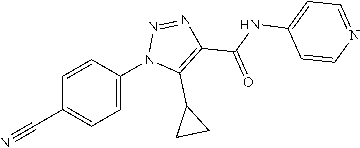 [SMILES: N#Cc1ccc(-n2nnc(C(=O)Nc3ccncc3)c2C2CC2)cc1] |
| 2-2 | 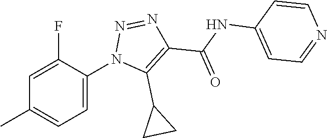 [SMILES: Cc1ccc(-n2nnc(C(=O)Nc3ccncc3)c2C2CC2)c(F)c1] |
| 2-3 | 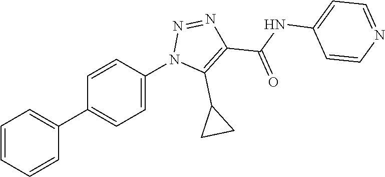 [SMILES: O=C(Nc1ccncc1)c1nnn(-c2ccc(-c3ccccc3)cc2)c1C1CC1] |
| 2-4 | 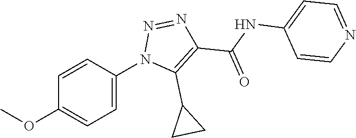 [SMILES: COc1ccc(-n2nnc(C(=O)Nc3ccncc3)c2C2CC2)cc1] |
| 2-5 | 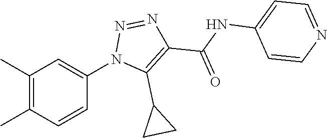 [SMILES: Cc1ccc(-n2nnc(C(=O)Nc3ccncc3)c2C2CC2)cc1C] |
| 2-6 | 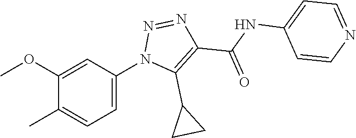 [SMILES: COc1cc(-n2nnc(C(=O)Nc3ccncc3)c2C2CC2)ccc1C] |
| 2-7 | 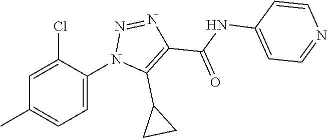 [SMILES: Cc1ccc(-n2nnc(C(=O)Nc3ccncc3)c2C2CC2)c(Cl)c1] |
| 2-8 | 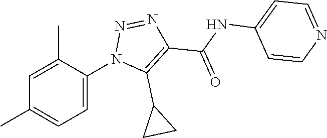 [SMILES: Cc1ccc(-n2nnc(C(=O)Nc3ccncc3)c2C2CC2)c(C)c1] |
| 2-9 | 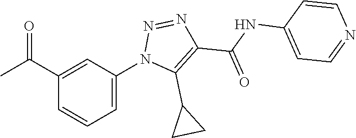 [SMILES: CC(=O)c1cccc(-n2nnc(C(=O)Nc3ccncc3)c2C2CC2)c1] |
| 2-10 | 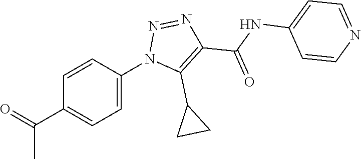 [SMILES: CC(=O)c1ccc(-n2nnc(C(=O)Nc3ccncc3)c2C2CC2)cc1] |
| 2-11 | 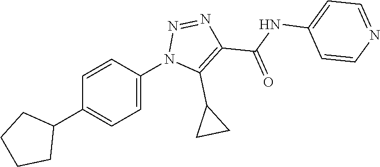 [SMILES: O=C(Nc1ccncc1)c1nnn(-c2ccc(C3CCCC3)cc2)c1C1CC1] |
| 2-13 | 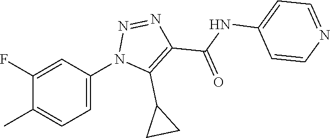 [SMILES: Cc1ccc(-n2nnc(C(=O)Nc3ccncc3)c2C2CC2)cc1F] |
| 2-14 | 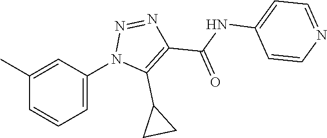 [SMILES: Cc1cccc(-n2nnc(C(=O)Nc3ccncc3)c2C2CC2)c1] |
| 3-1 | 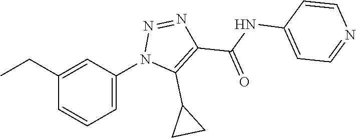 [SMILES: CCc1cccc(-n2nnc(C(=O)Nc3ccncc3)c2C2CC2)c1] |
| 3-2 | 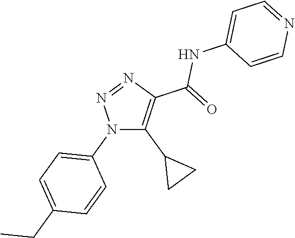 [SMILES: CCc1ccc(-n2nnc(C(=O)Nc3ccncc3)c2C2CC2)cc1] |
| 3-3 | 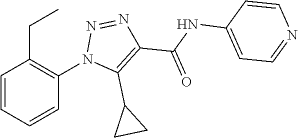 [SMILES: CCc1ccccc1-n1nnc(C(=O)Nc2ccncc2)c1C1CC1] |
| 3-4 | 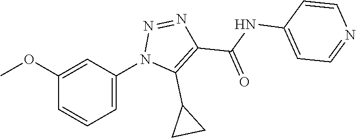 [SMILES: COc1cccc(-n2nnc(C(=O)Nc3ccncc3)c2C2CC2)c1] |
| 3-5 | 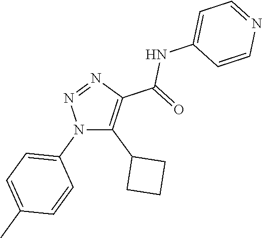 [SMILES: Cc1ccc(-n2nnc(C(=O)Nc3ccncc3)c2C2CCC2)cc1] |
| 3-6 | 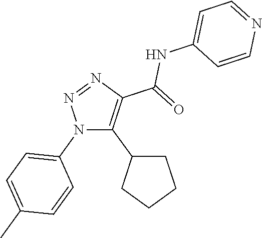 [SMILES: Cc1ccc(-n2nnc(C(=O)Nc3ccncc3)c2C2CCCC2)cc1] |
| 4-1 | 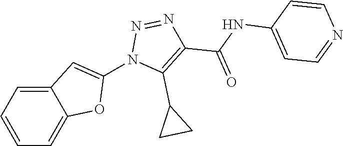 [SMILES: O=C(Nc1ccncc1)c1nnn(-c2cc3ccccc3o2)c1C1CC1] |
| 4-2 | 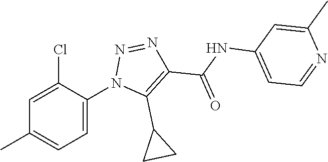 |
| 4-3 | 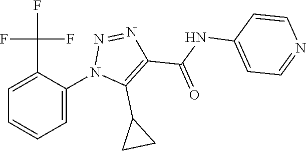 [SMILES: O=C(Nc1ccncc1)c1nnn(-c2ccccc2C(F)(F)F)c1C1CC1] |
| 4-4 | 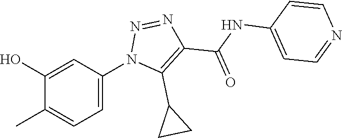 [SMILES: Cc1ccc(-n2nnc(C(=O)Nc3ccncc3)c2C2CC2)cc1O] |
| 4-5 | 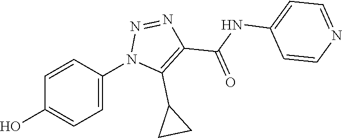 [SMILES: O=C(Nc1ccncc1)c1nnn(-c2ccc(O)cc2)c1C1CC1] |
| 4-6 | 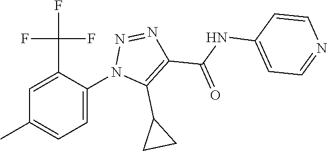 [SMILES: Cc1ccc(-n2nnc(C(=O)Nc3ccncc3)c2C2CC2)c(C(F)(F)F)c1] |
| 4-7 | 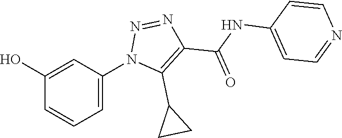 [SMILES: O=C(Nc1ccncc1)c1nnn(-c2cccc(O)c2)c1C1CC1] |
| 4-8 | 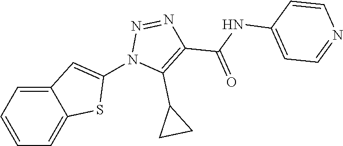 [SMILES: O=C(Nc1ccncc1)c1nnn(-c2cc3ccccc3s2)c1C1CC1] |
| 4-9 | 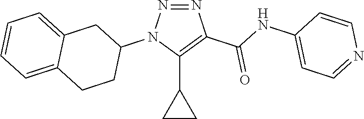 [SMILES: O=C(Nc1ccncc1)c1nnn(C2CCc3ccccc3C2)c1C1CC1] |
| 4-10 | 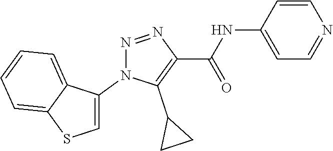 [SMILES: O=C(Nc1ccncc1)c1nnn(-c2csc3ccccc23)c1C1CC1] |
| 4-11 | 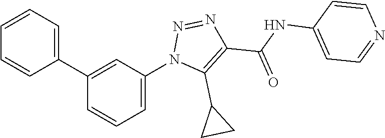 [SMILES: O=C(Nc1ccncc1)c1nnn(-c2cccc(-c3ccccc3)c2)c1C1CC1] |
| 4-12 | 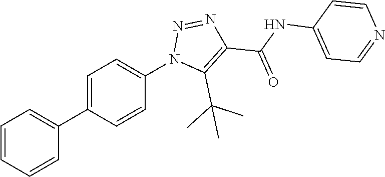 [SMILES: CC(C)(C)c1c(C(=O)Nc2ccncc2)nnn1-c1ccc(-c2ccccc2)cc1] |
| 4-13 | 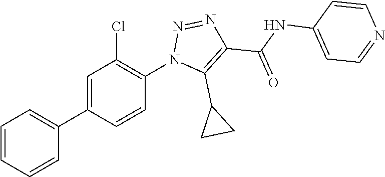 [SMILES: O=C(Nc1ccncc1)c1nnn(-c2ccc(-c3ccccc3)cc2Cl)c1C1CC1] |
| 5-1 | 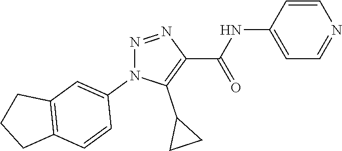 [SMILES: O=C(Nc1ccncc1)c1nnn(-c2ccc3c(c2)CCC3)c1C1CC1] |
| 5-2 | 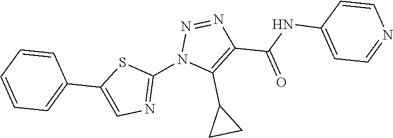 [SMILES: O=C(Nc1ccncc1)c1nnn(-c2ncc(-c3ccccc3)s2)c1C1CC1] |
| 5-3 | 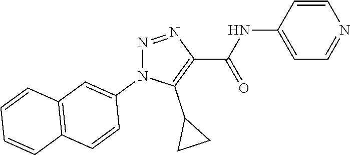 [SMILES: O=C(Nc1ccncc1)c1nnn(-c2ccc3ccccc3c2)c1C1CC1] |
| 5-4 | 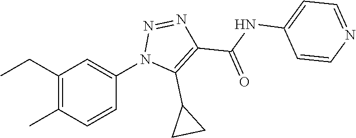 [SMILES: CCc1cc(-n2nnc(C(=O)Nc3ccncc3)c2C2CC2)ccc1C] |
| 5-5 | 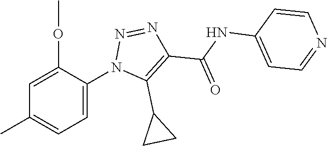 [SMILES: COc1cc(C)ccc1-n1nnc(C(=O)Nc2ccncc2)c1C1CC1] |
| 5-6 | 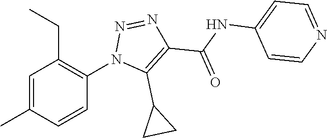 [SMILES: CCc1cc(C)ccc1-n1nnc(C(=O)Nc2ccncc2)c1C1CC1] |
| 5-7 | 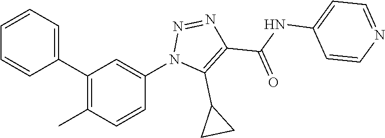 [SMILES: Cc1ccc(-n2nnc(C(=O)Nc3ccncc3)c2C2CC2)cc1-c1ccccc1] |
| 6-1 | 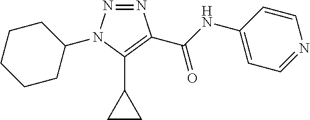 [SMILES: O=C(Nc1ccncc1)c1nnn(C2CCCCC2)c1C1CC1] |
| 6-2 | 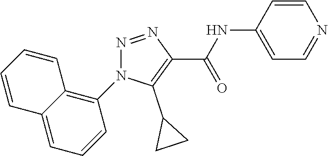 [SMILES: O=C(Nc1ccncc1)c1nnn(-c2cccc3ccccc23)c1C1CC1] |
| 6-3 | 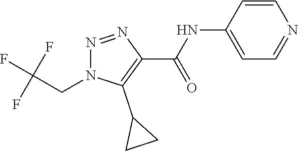 [SMILES: O=C(Nc1ccncc1)c1nnn(CC(F)(F)F)c1C1CC1] |
| 6-4 | 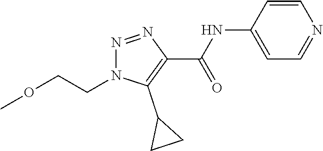 [SMILES: COCCn1nnc(C(=O)Nc2ccncc2)c1C1CC1] |
| 6-5 | 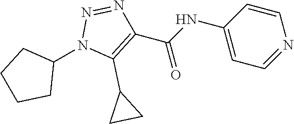 [SMILES: O=C(Nc1ccncc1)c1nnn(C2CCCC2)c1C1CC1] |
| 7-1 | 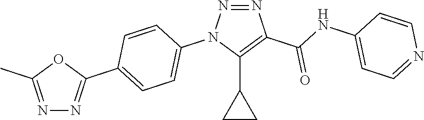 [SMILES: Cc1nnc(-c2ccc(-n3nnc(C(=O)Nc4ccncc4)c3C3CC3)cc2)o1] |
| 7-2 | 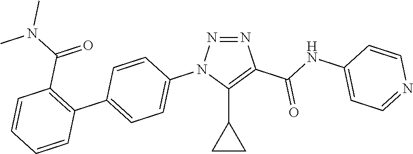 [SMILES: CN(C)C(=O)c1ccccc1-c1ccc(-n2nnc(C(=O)Nc3ccncc3)c2C2CC2)cc1] |
| 7-3 | 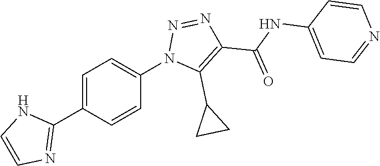 [SMILES: O=C(Nc1ccncc1)c1nnn(-c2ccc(-c3ncc[nH]3)cc2)c1C1CC1] |
| 7-4 | 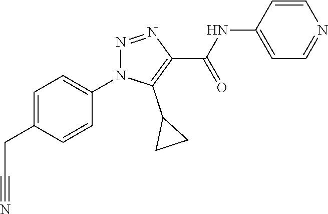 [SMILES: N#CCc1ccc(-n2nnc(C(=O)Nc3ccncc3)c2C2CC2)cc1] |
| 7-5 | 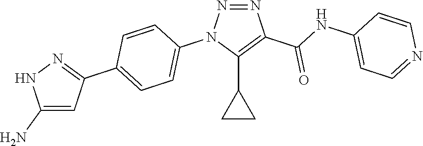 [SMILES: Nc1cc(-c2ccc(-n3nnc(C(=O)Nc4ccncc4)c3C3CC3)cc2)n[nH]1] |
| 7-7 | 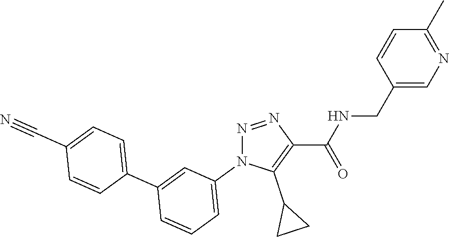 [SMILES: Cc1ccc(CNC(=O)c2nnn(-c3cccc(-c4ccc(C#N)cc4)c3)c2C2CC2)cn1] |
| 7-8 | 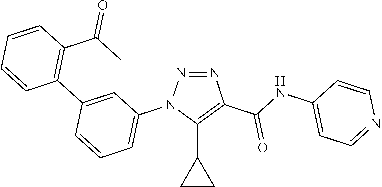 [SMILES: CC(=O)c1ccccc1-c1cccc(-n2nnc(C(=O)Nc3ccncc3)c2C2CC2)c1] |
| 7-9 | 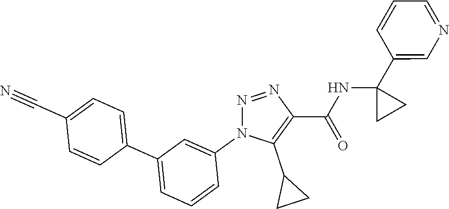 [SMILES: N#Cc1ccc(-c2cccc(-n3nnc(C(=O)NC4(c5cccnc5)CC4)c3C3CC3)c2)cc1] |
| 7-10 | 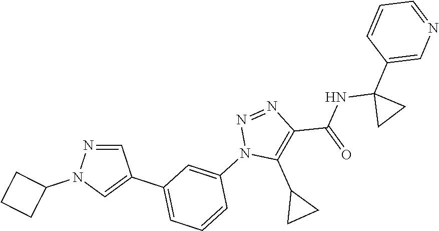 [SMILES: O=C(NC1(c2cccnc2)CC1)c1nnn(-c2cccc(-c3cnn(C4CCC4)c3)c2)c1C1CC1] |
| 7-11 | 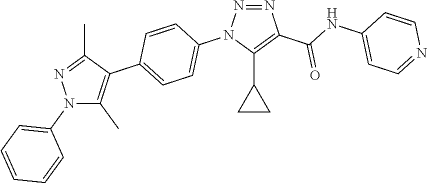 [SMILES: Cc1nn(-c2ccccc2)c(C)c1-c1ccc(-n2nnc(C(=O)Nc3ccncc3)c2C2CC2)cc1] |
| 7-12 | 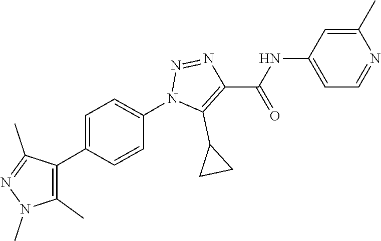 [SMILES: Cc1cc(NC(=O)c2nnn(-c3ccc(-c4c(C)nn(C)c4C)cc3)c2C2CC2)ccn1] |
| 7-13 | 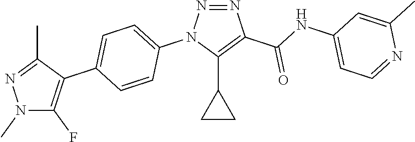 |
| 7-14 | 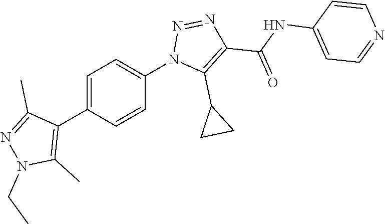 [SMILES: CCn1nc(C)c(-c2ccc(-n3nnc(C(=O)Nc4ccncc4)c3C3CC3)cc2)c1C] |
| 7-15 | 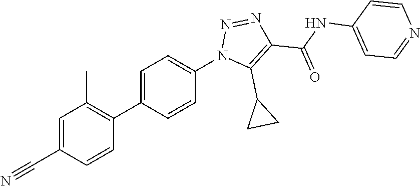 [SMILES: Cc1cc(C#N)ccc1-c1ccc(-n2nnc(C(=O)Nc3ccncc3)c2C2CC2)cc1] |
| 7-16 | 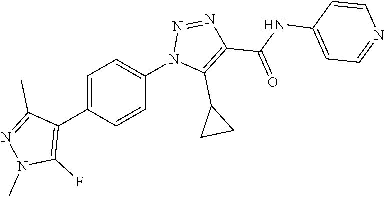 [SMILES: Cc1nn(C)c(F)c1-c1ccc(-n2nnc(C(=O)Nc3ccncc3)c2C2CC2)cc1] |
| 7-17 | 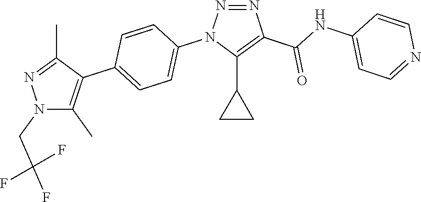 [SMILES: Cc1nn(CC(F)(F)F)c(C)c1-c1ccc(-n2nnc(C(=O)Nc3ccncc3)c2C2CC2)cc1] |
| 7-18 | 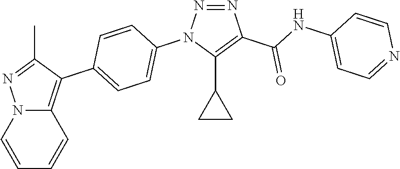 [SMILES: Cc1nn2ccccc2c1-c1ccc(-n2nnc(C(=O)Nc3ccncc3)c2C2CC2)cc1] |
| 7-19 | 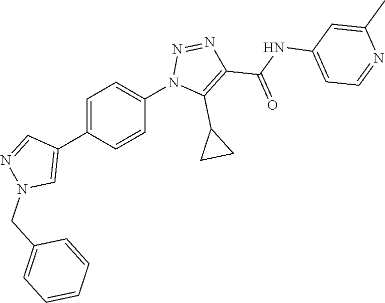 [SMILES: Cc1cc(NC(=O)c2nnn(-c3ccc(-c4cnn(Cc5ccccc5)c4)cc3)c2C2CC2)ccn1] |
| 7-20 | 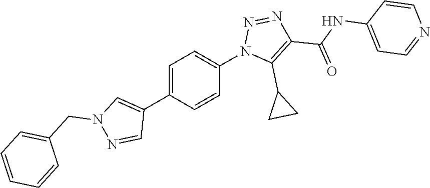 [SMILES: O=C(Nc1ccncc1)c1nnn(-c2ccc(-c3cnn(Cc4ccccc4)c3)cc2)c1C1CC1] |
| 7-21 | 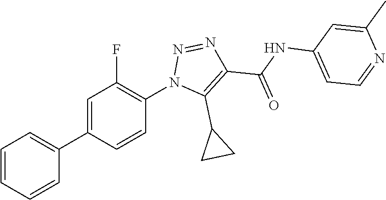 [SMILES: Cc1cc(NC(=O)c2nnn(-c3ccc(-c4ccccc4)cc3F)c2C2CC2)ccn1] |
| 7-22 | 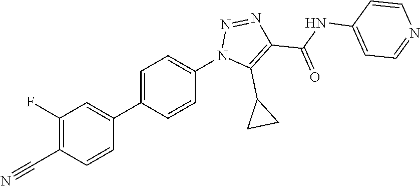 [SMILES: N#Cc1ccc(-c2ccc(-n3nnc(C(=O)Nc4ccncc4)c3C3CC3)cc2)cc1F] |
| 7-23 | 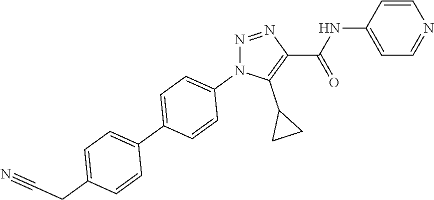 [SMILES: N#CCc1ccc(-c2ccc(-n3nnc(C(=O)Nc4ccncc4)c3C3CC3)cc2)cc1] |
| 7-24 | 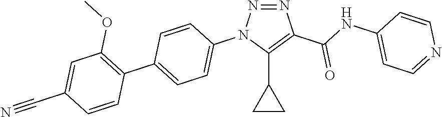 [SMILES: COc1cc(C#N)ccc1-c1ccc(-n2nnc(C(=O)Nc3ccncc3)c2C2CC2)cc1] |
| 7-25 | 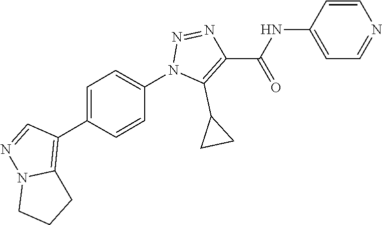 [SMILES: O=C(Nc1ccncc1)c1nnn(-c2ccc(-c3cnn4c3CCC4)cc2)c1C1CC1] |
| 7-26 | 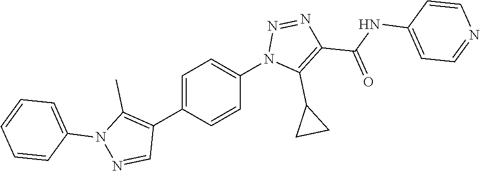 [SMILES: Cc1c(-c2ccc(-n3nnc(C(=O)Nc4ccncc4)c3C3CC3)cc2)cnn1-c1ccccc1] |
| 7-27 | 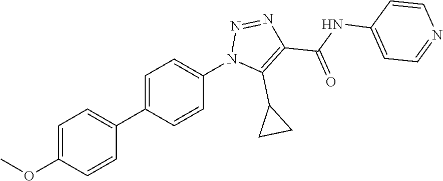 [SMILES: COc1ccc(-c2ccc(-n3nnc(C(=O)Nc4ccncc4)c3C3CC3)cc2)cc1] |
| 7-28 | 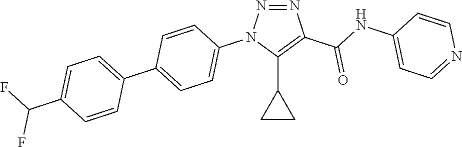 [SMILES: O=C(Nc1ccncc1)c1nnn(-c2ccc(-c3ccc(C(F)F)cc3)cc2)c1C1CC1] |
| 7-29 | 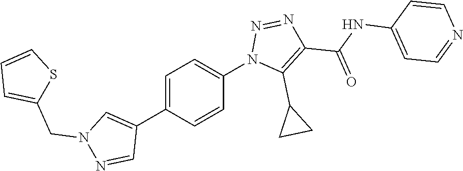 [SMILES: O=C(Nc1ccncc1)c1nnn(-c2ccc(-c3cnn(Cc4cccs4)c3)cc2)c1C1CC1] |
| 7-30 | 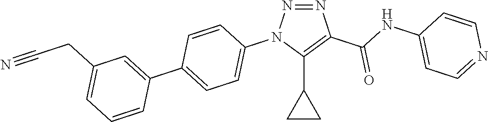 [SMILES: N#CCc1cccc(-c2ccc(-n3nnc(C(=O)Nc4ccncc4)c3C3CC3)cc2)c1] |
| 7-31 | 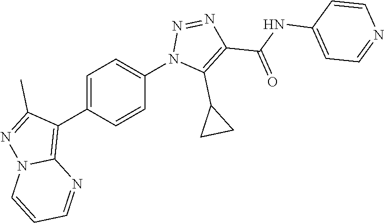 [SMILES: Cc1nn2cccnc2c1-c1ccc(-n2nnc(C(=O)Nc3ccncc3)c2C2CC2)cc1] |
| 7-32 | 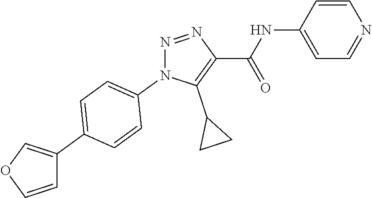 [SMILES: O=C(Nc1ccncc1)c1nnn(-c2ccc(-c3ccoc3)cc2)c1C1CC1] |
| 7-33 | 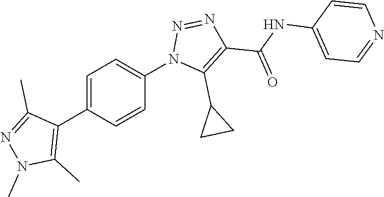 [SMILES: Cc1nn(C)c(C)c1-c1ccc(-n2nnc(C(=O)Nc3ccncc3)c2C2CC2)cc1] |
| 7-34 | 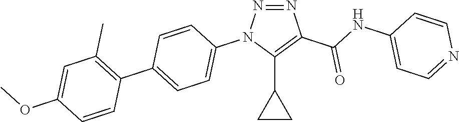 [SMILES: COc1ccc(-c2ccc(-n3nnc(C(=O)Nc4ccncc4)c3C3CC3)cc2)c(C)c1] |
| 7-35 | 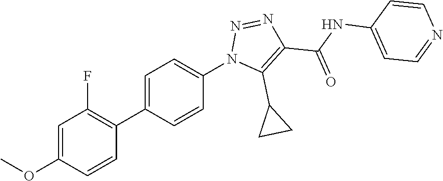 [SMILES: COc1ccc(-c2ccc(-n3nnc(C(=O)Nc4ccncc4)c3C3CC3)cc2)c(F)c1] |
| 7-36 | 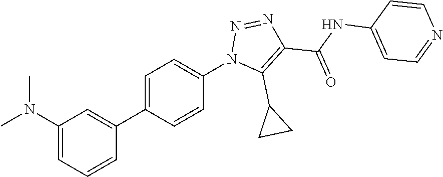 [SMILES: CN(C)c1cccc(-c2ccc(-n3nnc(C(=O)Nc4ccncc4)c3C3CC3)cc2)c1] |
| 7-37 | 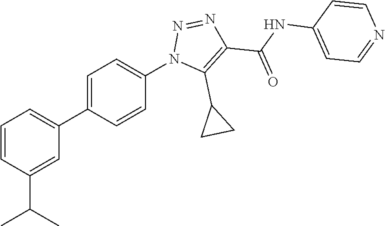 [SMILES: CC(C)c1cccc(-c2ccc(-n3nnc(C(=O)Nc4ccncc4)c3C3CC3)cc2)c1] |
| 7-38 | 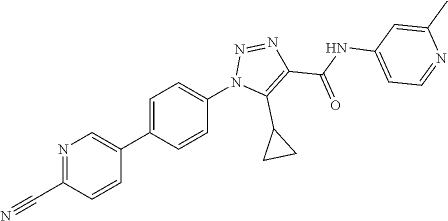 [SMILES: Cc1cc(NC(=O)c2nnn(-c3ccc(-c4ccc(C#N)nc4)cc3)c2C2CC2)ccn1] |
| 7-39 | 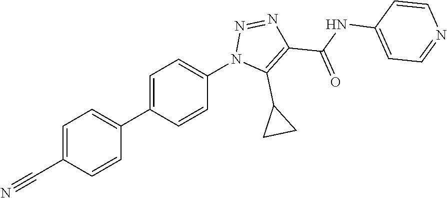 [SMILES: N#Cc1ccc(-c2ccc(-n3nnc(C(=O)Nc4ccncc4)c3C3CC3)cc2)cc1] |
| 7-40 | 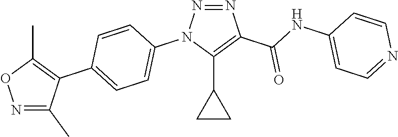 [SMILES: Cc1noc(C)c1-c1ccc(-n2nnc(C(=O)Nc3ccncc3)c2C2CC2)cc1] |
| 7-41 | 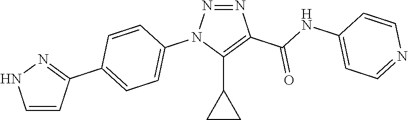 [SMILES: O=C(Nc1ccncc1)c1nnn(-c2ccc(-c3cc[nH]n3)cc2)c1C1CC1] |
| 7-42 | 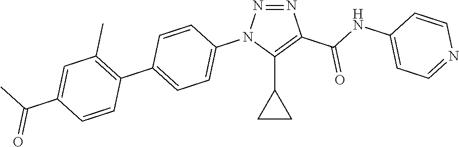 [SMILES: CC(=O)c1ccc(-c2ccc(-n3nnc(C(=O)Nc4ccncc4)c3C3CC3)cc2)c(C)c1] |
| 7-43 | 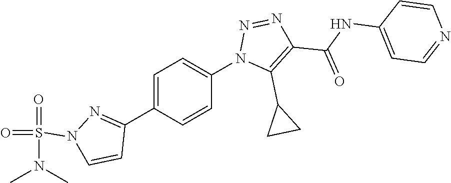 [SMILES: CN(C)S(=O)(=O)n1ccc(-c2ccc(-n3nnc(C(=O)Nc4ccncc4)c3C3CC3)cc2)n1] |
| 7-44 | 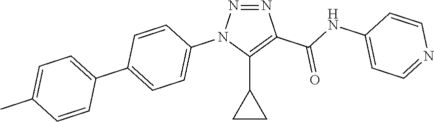 [SMILES: Cc1ccc(-c2ccc(-n3nnc(C(=O)Nc4ccncc4)c3C3CC3)cc2)cc1] |
| 7-45 | 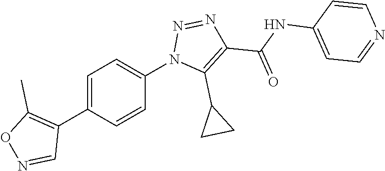 [SMILES: Cc1oncc1-c1ccc(-n2nnc(C(=O)Nc3ccncc3)c2C2CC2)cc1] |
| 7-46 | 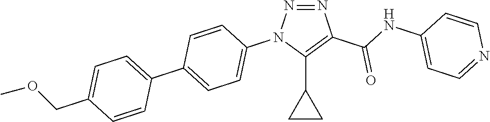 [SMILES: COCc1ccc(-c2ccc(-n3nnc(C(=O)Nc4ccncc4)c3C3CC3)cc2)cc1] |
| 7-47 | 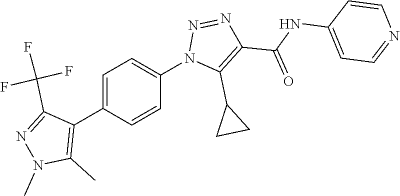 [SMILES: Cc1c(-c2ccc(-n3nnc(C(=O)Nc4ccncc4)c3C3CC3)cc2)c(C(F)(F)F)nn1C] |
| 7-48 | 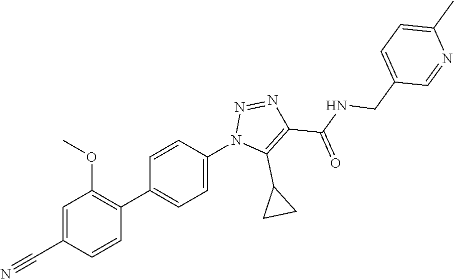 [SMILES: COc1cc(C#N)ccc1-c1ccc(-n2nnc(C(=O)NCc3ccc(C)nc3)c2C2CC2)cc1] |
| 7-49 | 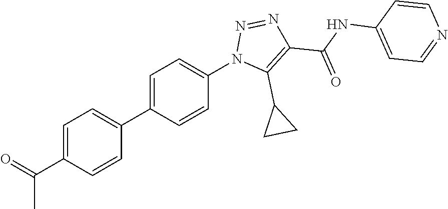 [SMILES: CC(=O)c1ccc(-c2ccc(-n3nnc(C(=O)Nc4ccncc4)c3C3CC3)cc2)cc1] |
| 7-50 | 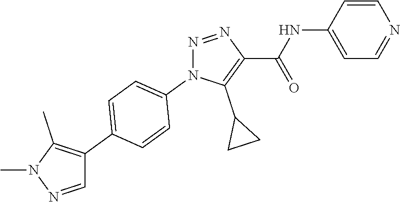 [SMILES: Cc1c(-c2ccc(-n3nnc(C(=O)Nc4ccncc4)c3C3CC3)cc2)cnn1C] |
| 7-51 | 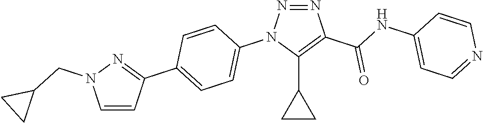 [SMILES: O=C(Nc1ccncc1)c1nnn(-c2ccc(-c3ccn(CC4CC4)n3)cc2)c1C1CC1] |
| 7-52 | 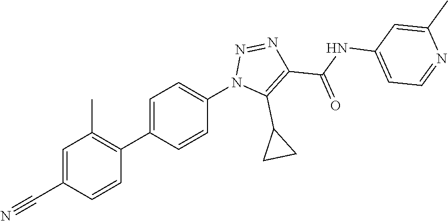 [SMILES: Cc1cc(NC(=O)c2nnn(-c3ccc(-c4ccc(C#N)cc4C)cc3)c2C2CC2)ccn1] |
| 7-53 | 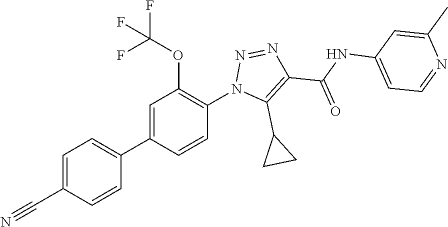 [SMILES: Cc1cc(NC(=O)c2nnn(-c3ccc(-c4ccc(C#N)cc4)cc3OC(F)(F)F)c2C2CC2)ccn1] |
| 7-54 | 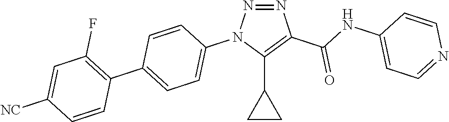 [SMILES: N#Cc1ccc(-c2ccc(-n3nnc(C(=O)Nc4ccncc4)c3C3CC3)cc2)c(F)c1] |
| 7-55 | 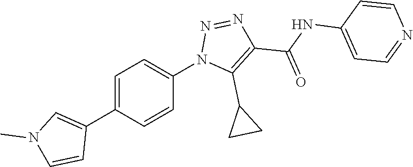 [SMILES: Cn1ccc(-c2ccc(-n3nnc(C(=O)Nc4ccncc4)c3C3CC3)cc2)c1] |
| 7-56 | 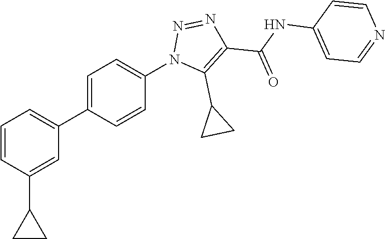 [SMILES: O=C(Nc1ccncc1)c1nnn(-c2ccc(-c3cccc(C4CC4)c3)cc2)c1C1CC1] |
| 7-57 | 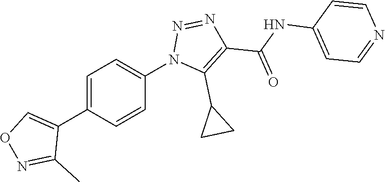 [SMILES: Cc1nocc1-c1ccc(-n2nnc(C(=O)Nc3ccncc3)c2C2CC2)cc1] |
| 7-58 | 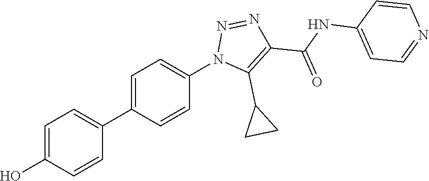 [SMILES: O=C(Nc1ccncc1)c1nnn(-c2ccc(-c3ccc(O)cc3)cc2)c1C1CC1] |
| 7-59 | 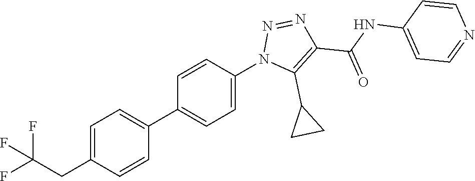 [SMILES: O=C(Nc1ccncc1)c1nnn(-c2ccc(-c3ccc(CC(F)(F)F)cc3)cc2)c1C1CC1] |
| 7-60 | 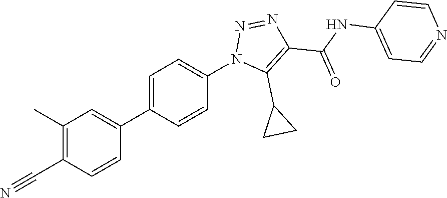 [SMILES: Cc1cc(-c2ccc(-n3nnc(C(=O)Nc4ccncc4)c3C3CC3)cc2)ccc1C#N] |
| 7-61 | 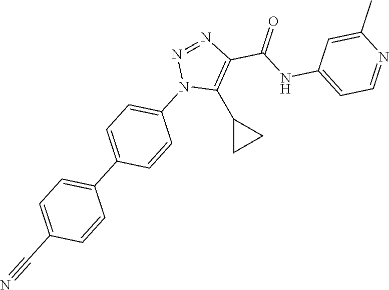 [SMILES: Cc1cc(NC(=O)c2nnn(-c3ccc(-c4ccc(C#N)cc4)cc3)c2C2CC2)ccn1] |
| 7-62 | 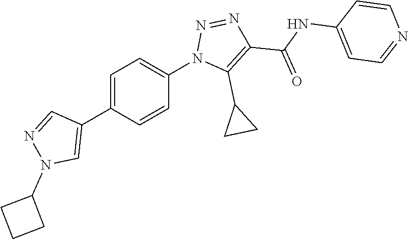 [SMILES: O=C(Nc1ccncc1)c1nnn(-c2ccc(-c3cnn(C4CCC4)c3)cc2)c1C1CC1] |
| 7-63 | 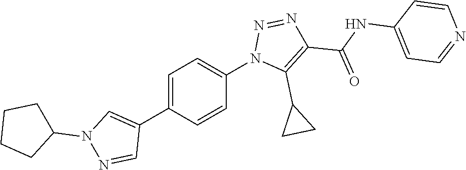 [SMILES: O=C(Nc1ccncc1)c1nnn(-c2ccc(-c3cnn(C4CCCC4)c3)cc2)c1C1CC1] |
| 7-64 | 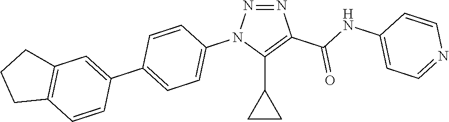 [SMILES: O=C(Nc1ccncc1)c1nnn(-c2ccc(-c3ccc4c(c3)CCC4)cc2)c1C1CC1] |
| 7-65 | 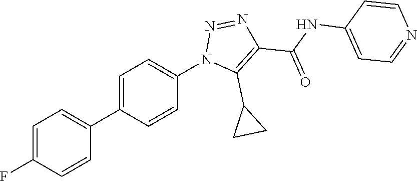 [SMILES: O=C(Nc1ccncc1)c1nnn(-c2ccc(-c3ccc(F)cc3)cc2)c1C1CC1] |
| 7-66 | 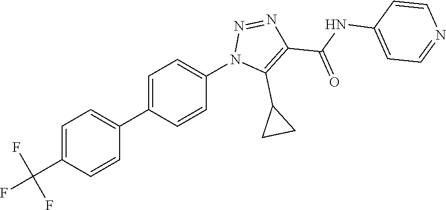 [SMILES: O=C(Nc1ccncc1)c1nnn(-c2ccc(-c3ccc(C(F)(F)F)cc3)cc2)c1C1CC1] |
| 7-67 | 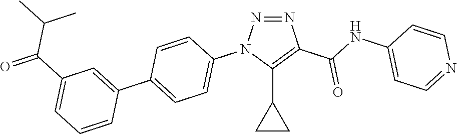 [SMILES: CC(C)C(=O)c1cccc(-c2ccc(-n3nnc(C(=O)Nc4ccncc4)c3C3CC3)cc2)c1] |
| 7-68 | 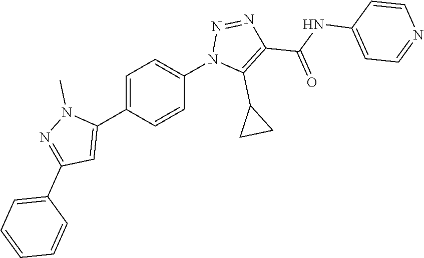 [SMILES: Cn1nc(-c2ccccc2)cc1-c1ccc(-n2nnc(C(=O)Nc3ccncc3)c2C2CC2)cc1] |
| 7-69 | 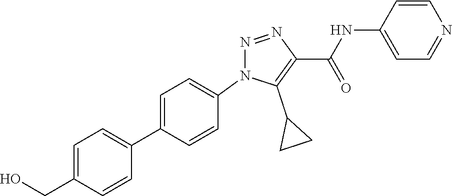 [SMILES: O=C(Nc1ccncc1)c1nnn(-c2ccc(-c3ccc(CO)cc3)cc2)c1C1CC1] |
| 7-70 | 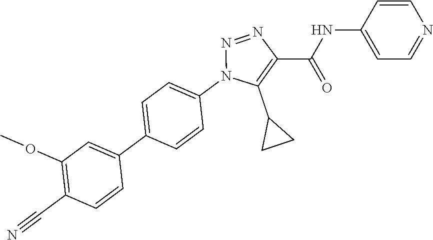 [SMILES: COc1cc(-c2ccc(-n3nnc(C(=O)Nc4ccncc4)c3C3CC3)cc2)ccc1C#N] |
| 7-71 | 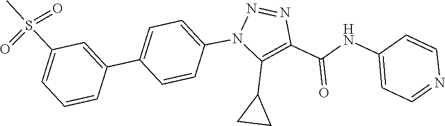 [SMILES: CS(=O)(=O)c1cccc(-c2ccc(-n3nnc(C(=O)Nc4ccncc4)c3C3CC3)cc2)c1] |
| 7-72 | 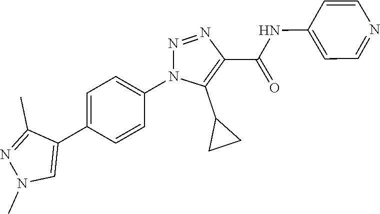 [SMILES: Cc1nn(C)cc1-c1ccc(-n2nnc(C(=O)Nc3ccncc3)c2C2CC2)cc1] |
| 7-73 | 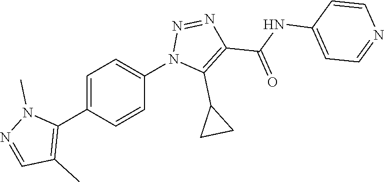 [SMILES: Cc1cnn(C)c1-c1ccc(-n2nnc(C(=O)Nc3ccncc3)c2C2CC2)cc1] |
| 7-74 | 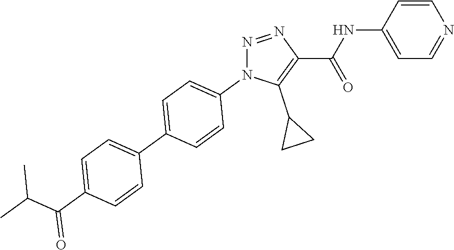 [SMILES: CC(C)C(=O)c1ccc(-c2ccc(-n3nnc(C(=O)Nc4ccncc4)c3C3CC3)cc2)cc1] |
| 7-75 | 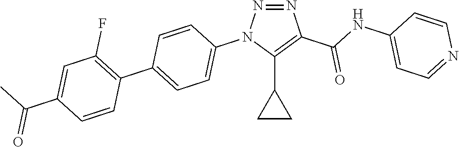 [SMILES: CC(=O)c1ccc(-c2ccc(-n3nnc(C(=O)Nc4ccncc4)c3C3CC3)cc2)c(F)c1] |
| 7-76 | 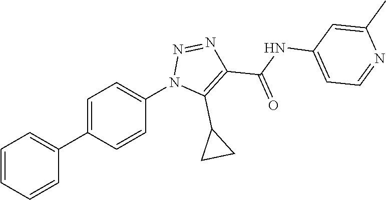 [SMILES: Cc1cc(NC(=O)c2nnn(-c3ccc(-c4ccccc4)cc3)c2C2CC2)ccn1] |
| 7-77 | 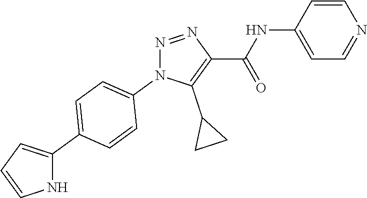 [SMILES: O=C(Nc1ccncc1)c1nnn(-c2ccc(-c3ccc[nH]3)cc2)c1C1CC1] |
| 7-78 | 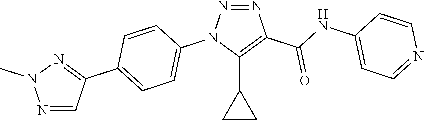 [SMILES: Cn1ncc(-c2ccc(-n3nnc(C(=O)Nc4ccncc4)c3C3CC3)cc2)n1] |
| 7-79 | 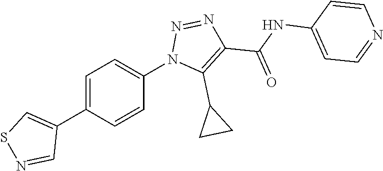 [SMILES: O=C(Nc1ccncc1)c1nnn(-c2ccc(-c3cnsc3)cc2)c1C1CC1] |
| 7-80 | 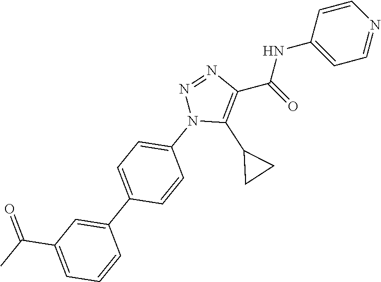 [SMILES: CC(=O)c1cccc(-c2ccc(-n3nnc(C(=O)Nc4ccncc4)c3C3CC3)cc2)c1] |
| 7-81 | 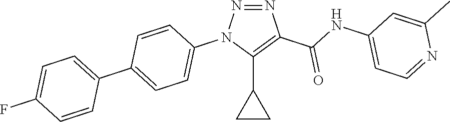 [SMILES: Cc1cc(NC(=O)c2nnn(-c3ccc(-c4ccc(F)cc4)cc3)c2C2CC2)ccn1] |
| 7-82 | 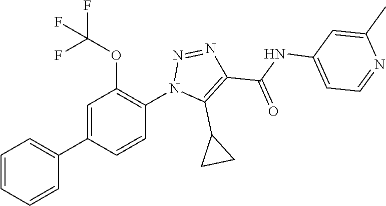 [SMILES: Cc1cc(NC(=O)c2nnn(-c3ccc(-c4ccccc4)cc3OC(F)(F)F)c2C2CC2)ccn1] |
| 7-83 | 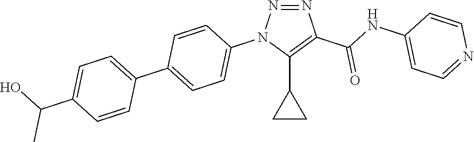 [SMILES: CC(O)c1ccc(-c2ccc(-n3nnc(C(=O)Nc4ccncc4)c3C3CC3)cc2)cc1] |
| 7-84 | 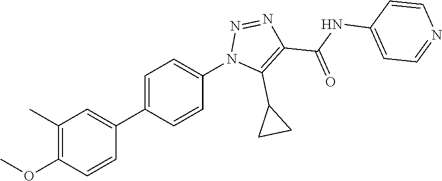 [SMILES: COc1ccc(-c2ccc(-n3nnc(C(=O)Nc4ccncc4)c3C3CC3)cc2)cc1C] |
| 7-85 | 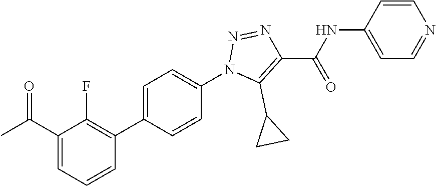 [SMILES: CC(=O)c1cccc(-c2ccc(-n3nnc(C(=O)Nc4ccncc4)c3C3CC3)cc2)c1F] |
| 7-86 | 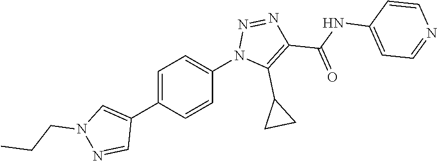 [SMILES: CCCn1cc(-c2ccc(-n3nnc(C(=O)Nc4ccncc4)c3C3CC3)cc2)cn1] |
| 7-87 | 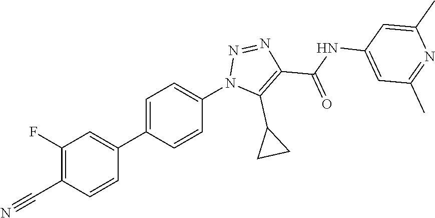 [SMILES: Cc1cc(NC(=O)c2nnn(-c3ccc(-c4ccc(C#N)c(F)c4)cc3)c2C2CC2)cc(C)n1] |
| 7-88 | 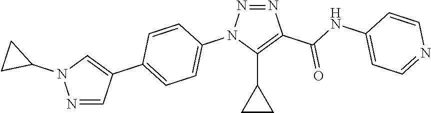 [SMILES: O=C(Nc1ccncc1)c1nnn(-c2ccc(-c3cnn(C4CC4)c3)cc2)c1C1CC1] |
| 7-89 | 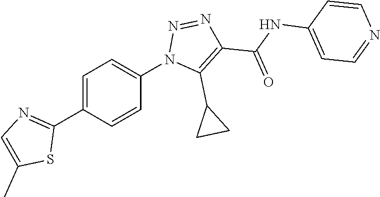 [SMILES: Cc1cnc(-c2ccc(-n3nnc(C(=O)Nc4ccncc4)c3C3CC3)cc2)s1] |
| 7-90 | 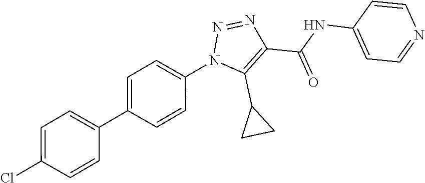 [SMILES: O=C(Nc1ccncc1)c1nnn(-c2ccc(-c3ccc(Cl)cc3)cc2)c1C1CC1] |
| 7-91 | 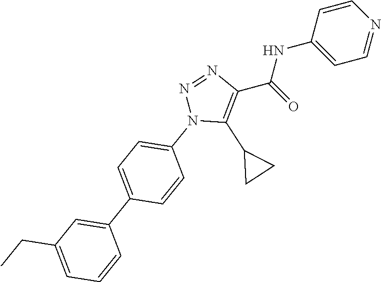 [SMILES: CCc1cccc(-c2ccc(-n3nnc(C(=O)Nc4ccncc4)c3C3CC3)cc2)c1] |
| 7-92 | 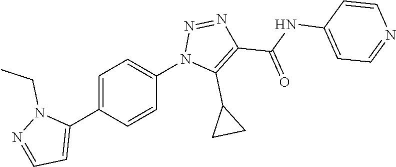 [SMILES: CCn1nccc1-c1ccc(-n2nnc(C(=O)Nc3ccncc3)c2C2CC2)cc1] |
| 7-93 | 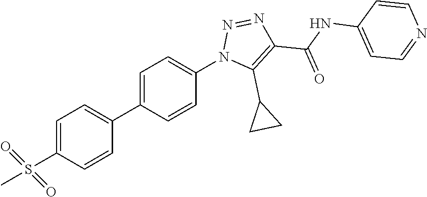 [SMILES: CS(=O)(=O)c1ccc(-c2ccc(-n3nnc(C(=O)Nc4ccncc4)c3C3CC3)cc2)cc1] |
| 7-94 | 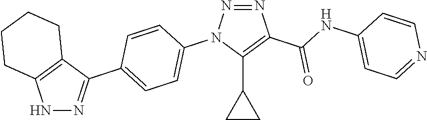 [SMILES: O=C(Nc1ccncc1)c1nnn(-c2ccc(-c3n[nH]c4c3CCCC4)cc2)c1C1CC1] |
| 7-95 | 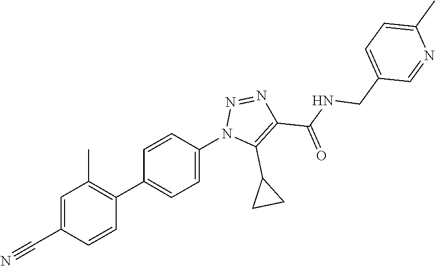 [SMILES: Cc1ccc(CNC(=O)c2nnn(-c3ccc(-c4ccc(C#N)cc4C)cc3)c2C2CC2)cn1] |
| 7-96 | 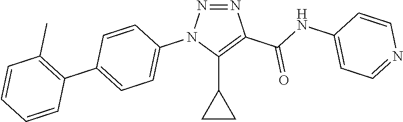 [SMILES: Cc1ccccc1-c1ccc(-n2nnc(C(=O)Nc3ccncc3)c2C2CC2)cc1] |
| 7-97 | 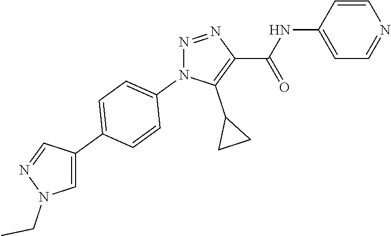 [SMILES: CCn1cc(-c2ccc(-n3nnc(C(=O)Nc4ccncc4)c3C3CC3)cc2)cn1] |
| 7-98 | 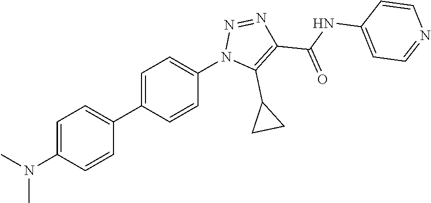 [SMILES: CN(C)c1ccc(-c2ccc(-n3nnc(C(=O)Nc4ccncc4)c3C3CC3)cc2)cc1] |
| 7-99 | 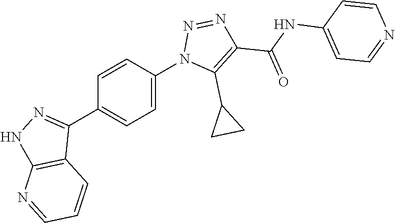 [SMILES: O=C(Nc1ccncc1)c1nnn(-c2ccc(-c3n[nH]c4ncccc34)cc2)c1C1CC1] |
| 7-100 | 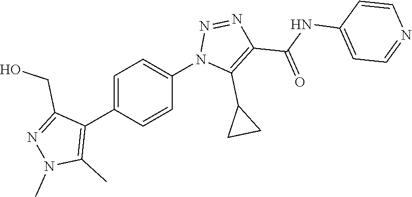 [SMILES: Cc1c(-c2ccc(-n3nnc(C(=O)Nc4ccncc4)c3C3CC3)cc2)c(CO)nn1C] |
| 7-101 | 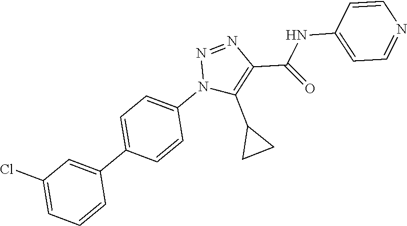 [SMILES: O=C(Nc1ccncc1)c1nnn(-c2ccc(-c3cccc(Cl)c3)cc2)c1C1CC1] |
| 7-102 | 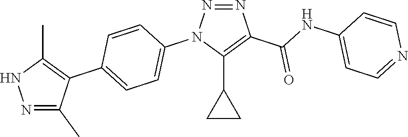 [SMILES: Cc1n[nH]c(C)c1-c1ccc(-n2nnc(C(=O)Nc3ccncc3)c2C2CC2)cc1] |
| 7-103 | 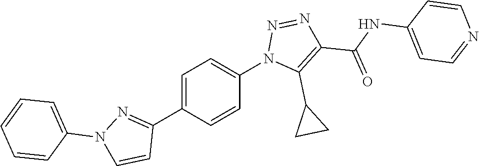 [SMILES: O=C(Nc1ccncc1)c1nnn(-c2ccc(-c3ccn(-c4ccccc4)n3)cc2)c1C1CC1] |
| 7-104 | 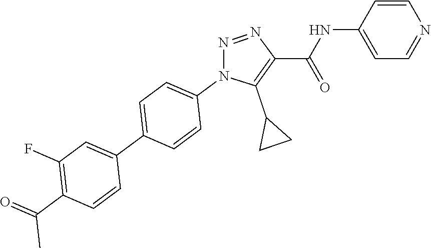 [SMILES: CC(=O)c1ccc(-c2ccc(-n3nnc(C(=O)Nc4ccncc4)c3C3CC3)cc2)cc1F] |
| 7-105 | 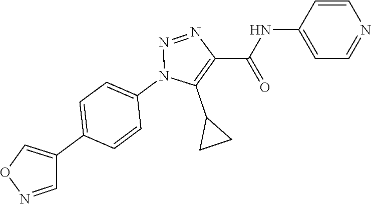 [SMILES: O=C(Nc1ccncc1)c1nnn(-c2ccc(-c3cnoc3)cc2)c1C1CC1] |
| 7-106 | 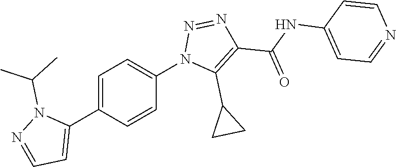 [SMILES: CC(C)n1nccc1-c1ccc(-n2nnc(C(=O)Nc3ccncc3)c2C2CC2)cc1] |
| 7-107 | 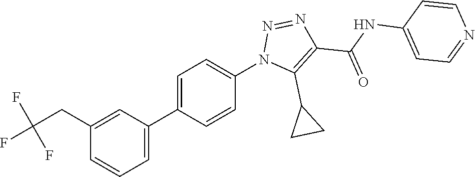 [SMILES: O=C(Nc1ccncc1)c1nnn(-c2ccc(-c3cccc(CC(F)(F)F)c3)cc2)c1C1CC1] |
| 7-108 | 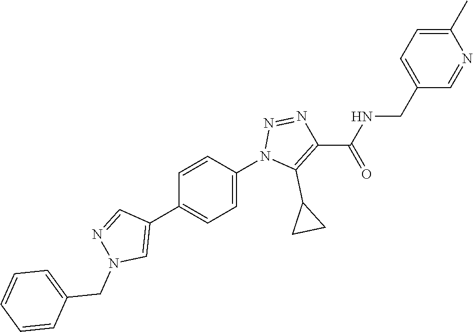 [SMILES: Cc1ccc(CNC(=O)c2nnn(-c3ccc(-c4cnn(Cc5ccccc5)c4)cc3)c2C2CC2)cn1] |
| 7-109 | 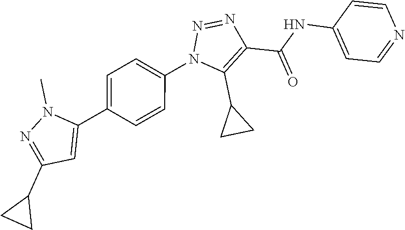 [SMILES: Cn1nc(C2CC2)cc1-c1ccc(-n2nnc(C(=O)Nc3ccncc3)c2C2CC2)cc1] |
| 7-110 | 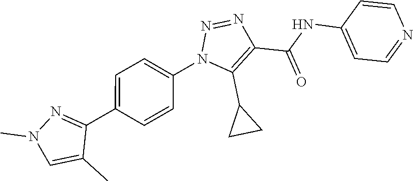 [SMILES: Cc1cn(C)nc1-c1ccc(-n2nnc(C(=O)Nc3ccncc3)c2C2CC2)cc1] |
| 7-111 | 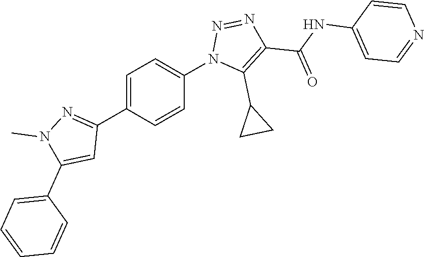 [SMILES: Cn1nc(-c2ccc(-n3nnc(C(=O)Nc4ccncc4)c3C3CC3)cc2)cc1-c1ccccc1] |
| 7-112 | 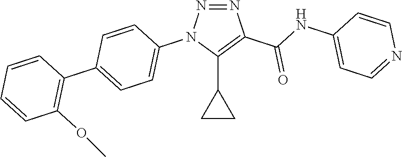 [SMILES: COc1ccccc1-c1ccc(-n2nnc(C(=O)Nc3ccncc3)c2C2CC2)cc1] |
| 7-113 | 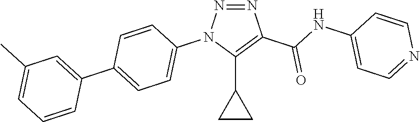 [SMILES: Cc1cccc(-c2ccc(-n3nnc(C(=O)Nc4ccncc4)c3C3CC3)cc2)c1] |
| 7-114 | 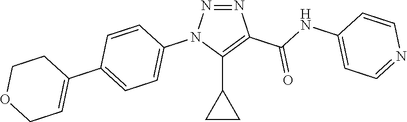 [SMILES: O=C(Nc1ccncc1)c1nnn(-c2ccc(C3=CCOCC3)cc2)c1C1CC1] |
| 7-115 | 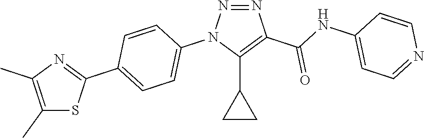 [SMILES: Cc1nc(-c2ccc(-n3nnc(C(=O)Nc4ccncc4)c3C3CC3)cc2)sc1C] |
| 7-116 | 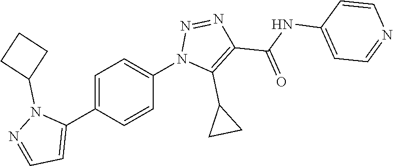 [SMILES: O=C(Nc1ccncc1)c1nnn(-c2ccc(-c3ccnn3C3CCC3)cc2)c1C1CC1] |
| 7-117 | 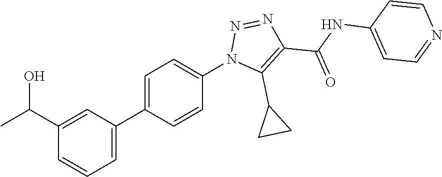 [SMILES: CC(O)c1cccc(-c2ccc(-n3nnc(C(=O)Nc4ccncc4)c3C3CC3)cc2)c1] |
| 7-118 | 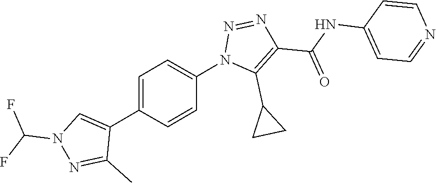 [SMILES: Cc1nn(C(F)F)cc1-c1ccc(-n2nnc(C(=O)Nc3ccncc3)c2C2CC2)cc1] |
| 7-119 | 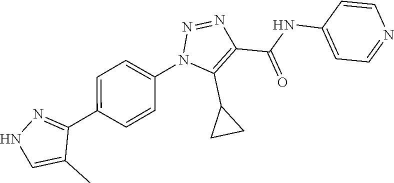 [SMILES: Cc1c[nH]nc1-c1ccc(-n2nnc(C(=O)Nc3ccncc3)c2C2CC2)cc1] |
| 7-120 | 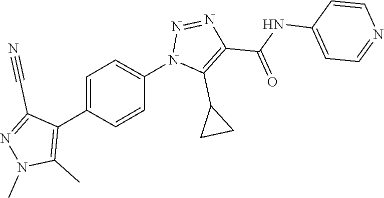 [SMILES: Cc1c(-c2ccc(-n3nnc(C(=O)Nc4ccncc4)c3C3CC3)cc2)c(C#N)nn1C] |
| 7-121 | 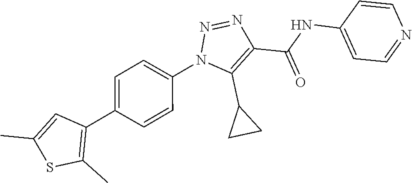 [SMILES: Cc1cc(-c2ccc(-n3nnc(C(=O)Nc4ccncc4)c3C3CC3)cc2)c(C)s1] |
| 7-122 | 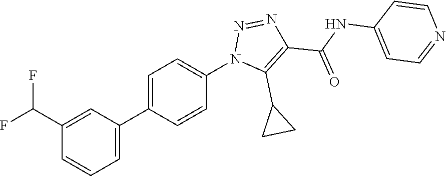 [SMILES: O=C(Nc1ccncc1)c1nnn(-c2ccc(-c3cccc(C(F)F)c3)cc2)c1C1CC1] |
| 7-123 | 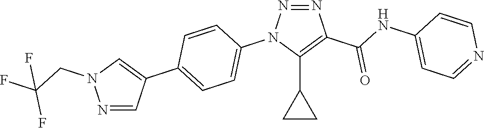 [SMILES: O=C(Nc1ccncc1)c1nnn(-c2ccc(-c3cnn(CC(F)(F)F)c3)cc2)c1C1CC1] |
| 7-124 | 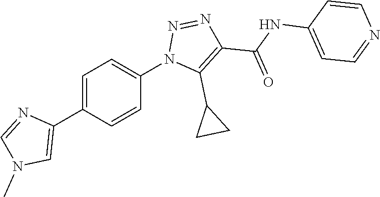 [SMILES: Cn1cnc(-c2ccc(-n3nnc(C(=O)Nc4ccncc4)c3C3CC3)cc2)c1] |
| 7-125 | 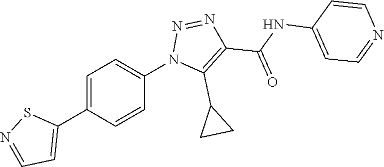 [SMILES: O=C(Nc1ccncc1)c1nnn(-c2ccc(-c3ccns3)cc2)c1C1CC1] |
| 7-126 | 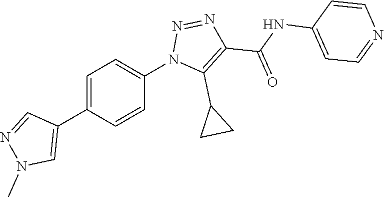 [SMILES: Cn1cc(-c2ccc(-n3nnc(C(=O)Nc4ccncc4)c3C3CC3)cc2)cn1] |
| 7-127 | 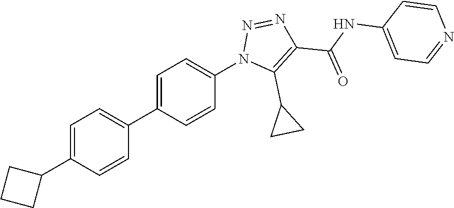 [SMILES: O=C(Nc1ccncc1)c1nnn(-c2ccc(-c3ccc(C4CCC4)cc3)cc2)c1C1CC1] |
| 7-128 | 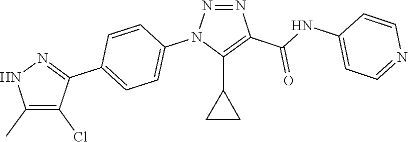 [SMILES: Cc1[nH]nc(-c2ccc(-n3nnc(C(=O)Nc4ccncc4)c3C3CC3)cc2)c1Cl] |
| 7-129 | 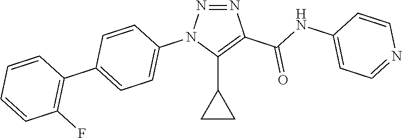 [SMILES: O=C(Nc1ccncc1)c1nnn(-c2ccc(-c3ccccc3F)cc2)c1C1CC1] |
| 7-130 | 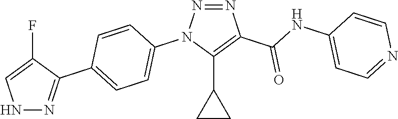 [SMILES: O=C(Nc1ccncc1)c1nnn(-c2ccc(-c3n[nH]cc3F)cc2)c1C1CC1] |
| 7-131 | 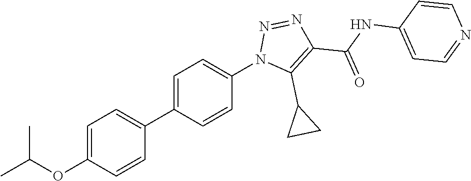 [SMILES: CC(C)Oc1ccc(-c2ccc(-n3nnc(C(=O)Nc4ccncc4)c3C3CC3)cc2)cc1] |
| 7-132 | 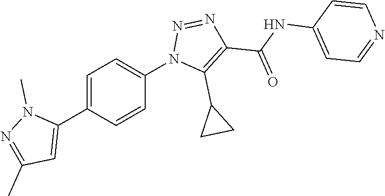 [SMILES: Cc1cc(-c2ccc(-n3nnc(C(=O)Nc4ccncc4)c3C3CC3)cc2)n(C)n1] |
| 7-133 | 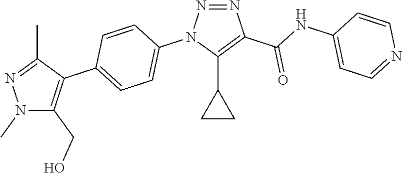 [SMILES: Cc1nn(C)c(CO)c1-c1ccc(-n2nnc(C(=O)Nc3ccncc3)c2C2CC2)cc1] |
| 7-134 | 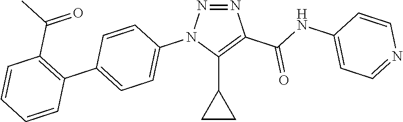 [SMILES: CC(=O)c1ccccc1-c1ccc(-n2nnc(C(=O)Nc3ccncc3)c2C2CC2)cc1] |
| 7-135 | 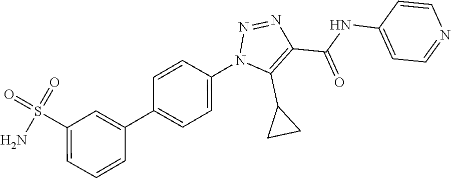 [SMILES: NS(=O)(=O)c1cccc(-c2ccc(-n3nnc(C(=O)Nc4ccncc4)c3C3CC3)cc2)c1] |
| 7-136 | 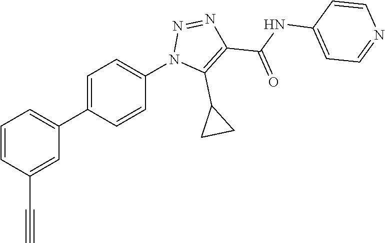 [SMILES: C#Cc1cccc(-c2ccc(-n3nnc(C(=O)Nc4ccncc4)c3C3CC3)cc2)c1] |
| 7-137 | 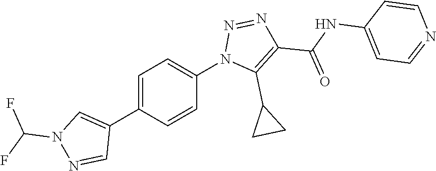 [SMILES: O=C(Nc1ccncc1)c1nnn(-c2ccc(-c3cnn(C(F)F)c3)cc2)c1C1CC1] |
| 7-138 | 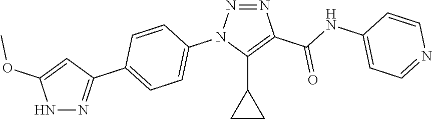 [SMILES: COc1cc(-c2ccc(-n3nnc(C(=O)Nc4ccncc4)c3C3CC3)cc2)n[nH]1] |
| 7-139 | 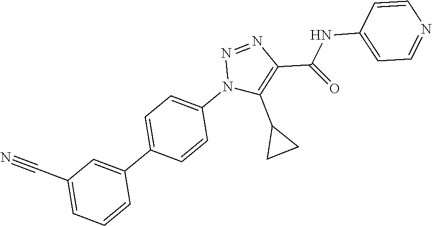 [SMILES: N#Cc1cccc(-c2ccc(-n3nnc(C(=O)Nc4ccncc4)c3C3CC3)cc2)c1] |
| 7-140 | 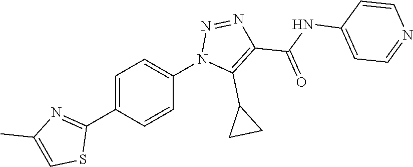 [SMILES: Cc1csc(-c2ccc(-n3nnc(C(=O)Nc4ccncc4)c3C3CC3)cc2)n1] |
| 7-141 | 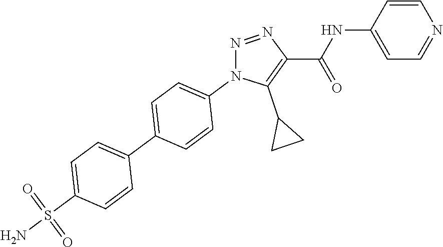 [SMILES: NS(=O)(=O)c1ccc(-c2ccc(-n3nnc(C(=O)Nc4ccncc4)c3C3CC3)cc2)cc1] |
| 7-142 | 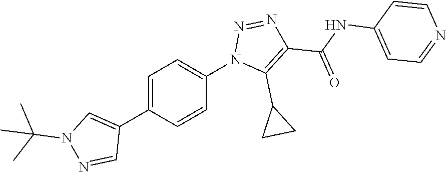 [SMILES: CC(C)(C)n1cc(-c2ccc(-n3nnc(C(=O)Nc4ccncc4)c3C3CC3)cc2)cn1] |
| 7-143 | 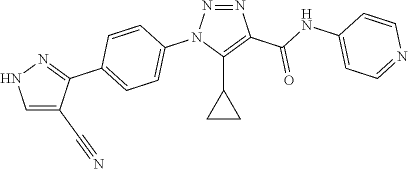 [SMILES: N#Cc1c[nH]nc1-c1ccc(-n2nnc(C(=O)Nc3ccncc3)c2C2CC2)cc1] |
| 7-144 | 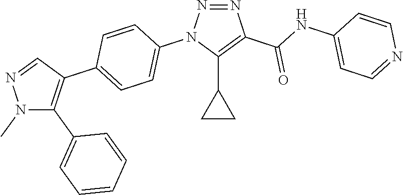 [SMILES: Cn1ncc(-c2ccc(-n3nnc(C(=O)Nc4ccncc4)c3C3CC3)cc2)c1-c1ccccc1] |
| 7-145 | 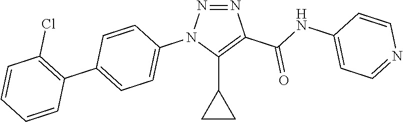 [SMILES: O=C(Nc1ccncc1)c1nnn(-c2ccc(-c3ccccc3Cl)cc2)c1C1CC1] |
| 7-146 | 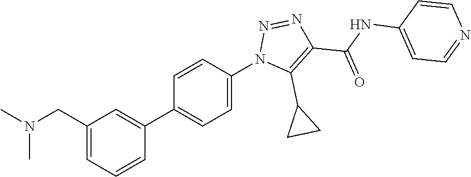 [SMILES: CN(C)Cc1cccc(-c2ccc(-n3nnc(C(=O)Nc4ccncc4)c3C3CC3)cc2)c1] |
| 7-147 | 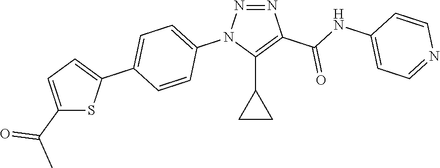 [SMILES: CC(=O)c1ccc(-c2ccc(-n3nnc(C(=O)Nc4ccncc4)c3C3CC3)cc2)s1] |
| 7-148 | 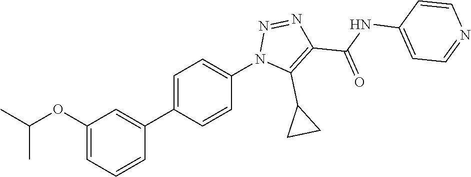 [SMILES: CC(C)Oc1cccc(-c2ccc(-n3nnc(C(=O)Nc4ccncc4)c3C3CC3)cc2)c1] |
| 7-149 | 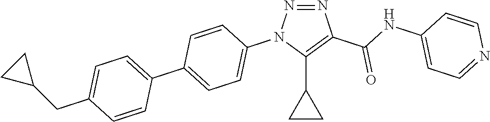 [SMILES: O=C(Nc1ccncc1)c1nnn(-c2ccc(-c3ccc(CC4CC4)cc3)cc2)c1C1CC1] |
| 7-150 | 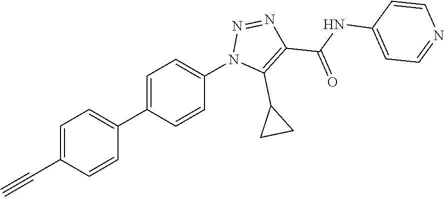 [SMILES: C#Cc1ccc(-c2ccc(-n3nnc(C(=O)Nc4ccncc4)c3C3CC3)cc2)cc1] |
| 7-151 | 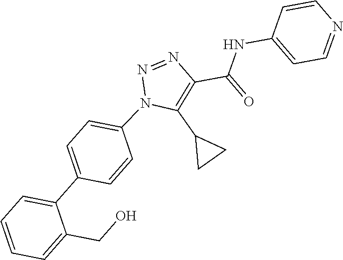 [SMILES: O=C(Nc1ccncc1)c1nnn(-c2ccc(-c3ccccc3CO)cc2)c1C1CC1] |
| 7-152 | 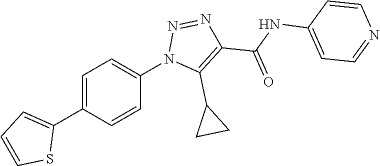 [SMILES: O=C(Nc1ccncc1)c1nnn(-c2ccc(-c3cccs3)cc2)c1C1CC1] |
| 7-153 | 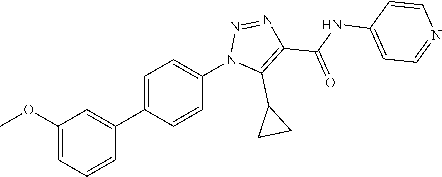 [SMILES: COc1cccc(-c2ccc(-n3nnc(C(=O)Nc4ccncc4)c3C3CC3)cc2)c1] |
| 7-154 | 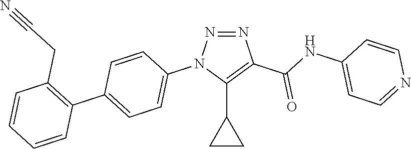 [SMILES: N#CCc1ccccc1-c1ccc(-n2nnc(C(=O)Nc3ccncc3)c2C2CC2)cc1] |
| 7-155 | 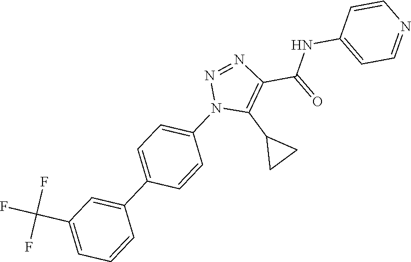 [SMILES: O=C(Nc1ccncc1)c1nnn(-c2ccc(-c3cccc(C(F)(F)F)c3)cc2)c1C1CC1] |
| 7-156 | 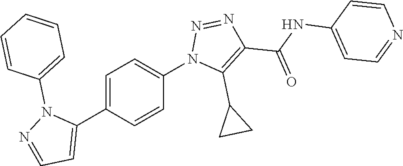 [SMILES: O=C(Nc1ccncc1)c1nnn(-c2ccc(-c3ccnn3-c3ccccc3)cc2)c1C1CC1] |
| 7-157 | 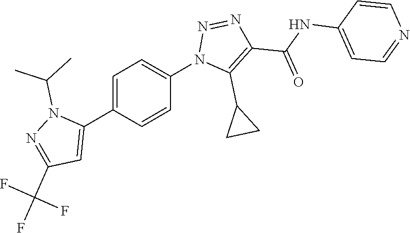 [SMILES: CC(C)n1nc(C(F)(F)F)cc1-c1ccc(-n2nnc(C(=O)Nc3ccncc3)c2C2CC2)cc1] |
| 7-158 | 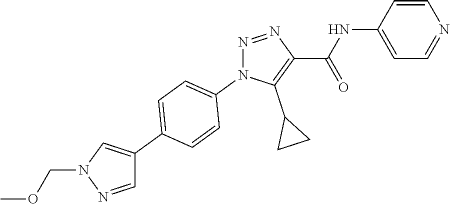 [SMILES: COCn1cc(-c2ccc(-n3nnc(C(=O)Nc4ccncc4)c3C3CC3)cc2)cn1] |
| 7-159 | 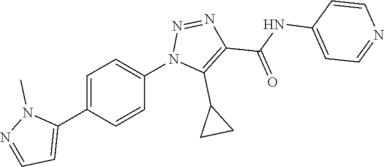 [SMILES: Cn1nccc1-c1ccc(-n2nnc(C(=O)Nc3ccncc3)c2C2CC2)cc1] |
| 7-160 | 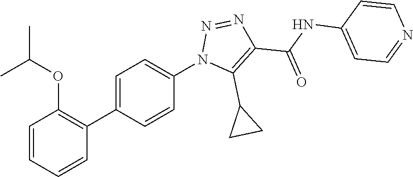 [SMILES: CC(C)Oc1ccccc1-c1ccc(-n2nnc(C(=O)Nc3ccncc3)c2C2CC2)cc1] |
| 7-161 | 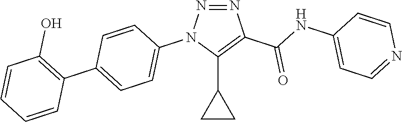 [SMILES: O=C(Nc1ccncc1)c1nnn(-c2ccc(-c3ccccc3O)cc2)c1C1CC1] |
| 7-162 | 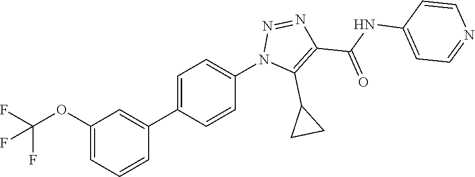 [SMILES: O=C(Nc1ccncc1)c1nnn(-c2ccc(-c3cccc(OC(F)(F)F)c3)cc2)c1C1CC1] |
| 7-163 | 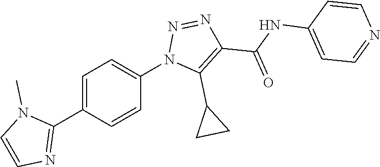 [SMILES: Cn1ccnc1-c1ccc(-n2nnc(C(=O)Nc3ccncc3)c2C2CC2)cc1] |
| 7-164 | 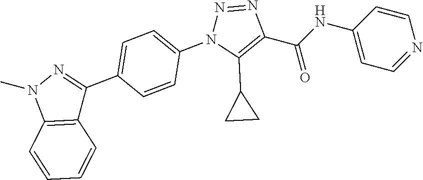 [SMILES: Cn1nc(-c2ccc(-n3nnc(C(=O)Nc4ccncc4)c3C3CC3)cc2)c2ccccc21] |
| 7-165 | 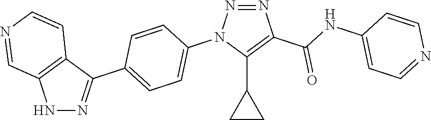 [SMILES: O=C(Nc1ccncc1)c1nnn(-c2ccc(-c3n[nH]c4cnccc34)cc2)c1C1CC1] |
| 7-166 | 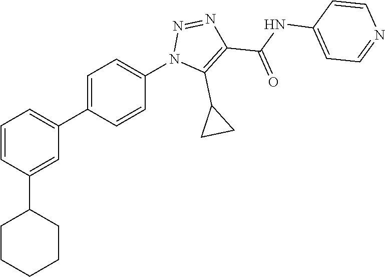 [SMILES: O=C(Nc1ccncc1)c1nnn(-c2ccc(-c3cccc(C4CCCCC4)c3)cc2)c1C1CC1] |
| 7-167 | 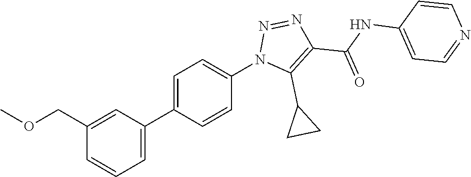 [SMILES: COCc1cccc(-c2ccc(-n3nnc(C(=O)Nc4ccncc4)c3C3CC3)cc2)c1] |
| 7-168 | 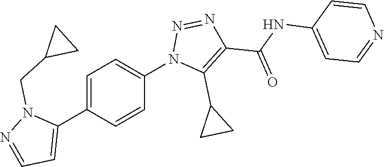 [SMILES: O=C(Nc1ccncc1)c1nnn(-c2ccc(-c3ccnn3CC3CC3)cc2)c1C1CC1] |
| 7-169 | 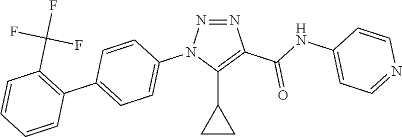 [SMILES: O=C(Nc1ccncc1)c1nnn(-c2ccc(-c3ccccc3C(F)(F)F)cc2)c1C1CC1] |
| 7-170 | 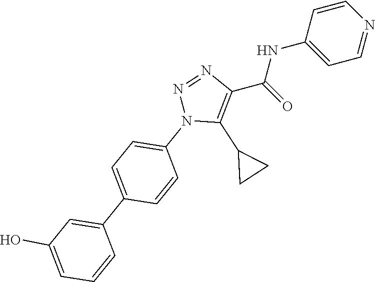 [SMILES: O=C(Nc1ccncc1)c1nnn(-c2ccc(-c3cccc(O)c3)cc2)c1C1CC1] |
| 7-171 | 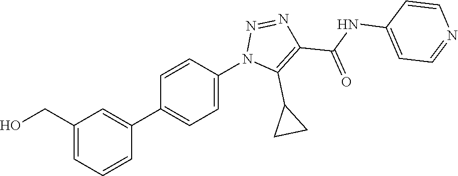 [SMILES: O=C(Nc1ccncc1)c1nnn(-c2ccc(-c3cccc(CO)c3)cc2)c1C1CC1] |
| 7-172 | 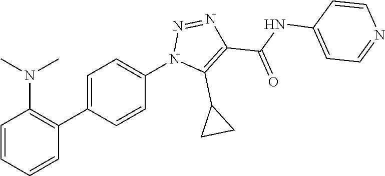 [SMILES: CN(C)c1ccccc1-c1ccc(-n2nnc(C(=O)Nc3ccncc3)c2C2CC2)cc1] |
| 7-173 | 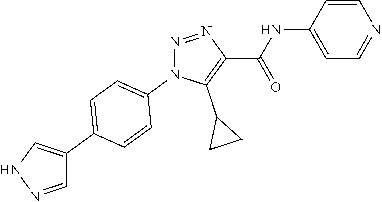 [SMILES: O=C(Nc1ccncc1)c1nnn(-c2ccc(-c3cn[nH]c3)cc2)c1C1CC1] |
| 7-174 | 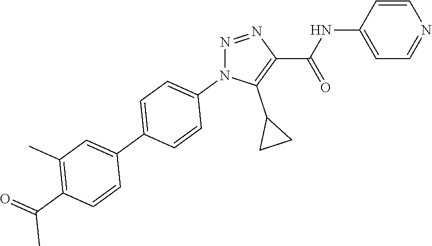 [SMILES: CC(=O)c1ccc(-c2ccc(-n3nnc(C(=O)Nc4ccncc4)c3C3CC3)cc2)cc1C] |
| 7-175 | 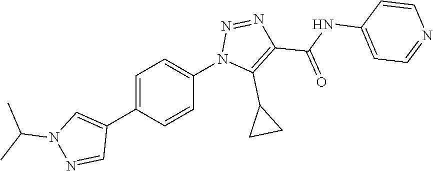 [SMILES: CC(C)n1cc(-c2ccc(-n3nnc(C(=O)Nc4ccncc4)c3C3CC3)cc2)cn1] |
| 7-176 | 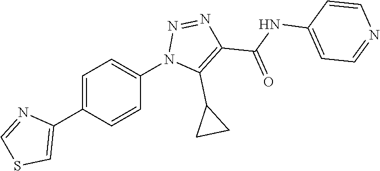 [SMILES: O=C(Nc1ccncc1)c1nnn(-c2ccc(-c3cscn3)cc2)c1C1CC1] |
| 7-177 | 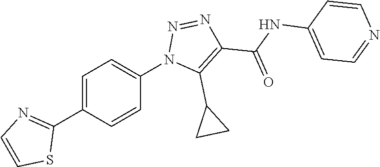 [SMILES: O=C(Nc1ccncc1)c1nnn(-c2ccc(-c3nccs3)cc2)c1C1CC1] |
| 7-178 | 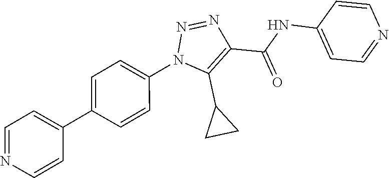 [SMILES: O=C(Nc1ccncc1)c1nnn(-c2ccc(-c3ccncc3)cc2)c1C1CC1] |
| 7-179 | 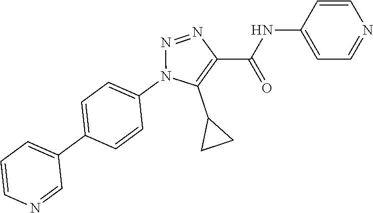 [SMILES: O=C(Nc1ccncc1)c1nnn(-c2ccc(-c3cccnc3)cc2)c1C1CC1] |
| 7-180 | 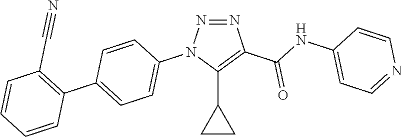 [SMILES: N#Cc1ccccc1-c1ccc(-n2nnc(C(=O)Nc3ccncc3)c2C2CC2)cc1] |
| 7-181 | 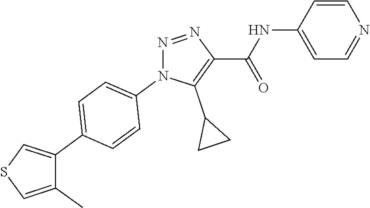 [SMILES: Cc1cscc1-c1ccc(-n2nnc(C(=O)Nc3ccncc3)c2C2CC2)cc1] |
| 7-182 | 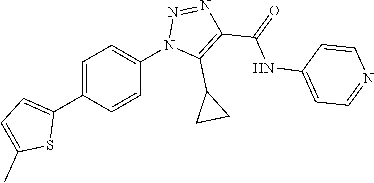 [SMILES: Cc1ccc(-c2ccc(-n3nnc(C(=O)Nc4ccncc4)c3C3CC3)cc2)s1] |
| 7-183 | 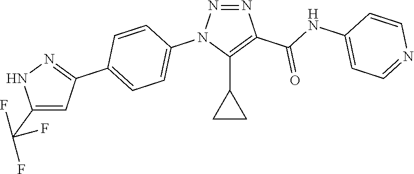 [SMILES: O=C(Nc1ccncc1)c1nnn(-c2ccc(-c3cc(C(F)(F)F)[nH]n3)cc2)c1C1CC1] |
| 7-184 | 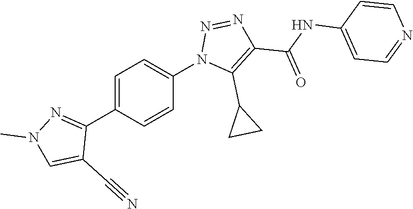 [SMILES: Cn1cc(C#N)c(-c2ccc(-n3nnc(C(=O)Nc4ccncc4)c3C3CC3)cc2)n1] |
| 7-185 | 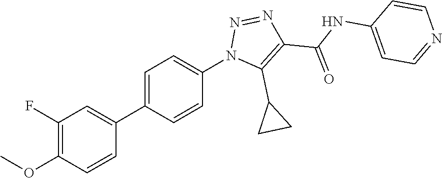 [SMILES: COc1ccc(-c2ccc(-n3nnc(C(=O)Nc4ccncc4)c3C3CC3)cc2)cc1F] |
| 7-186 | 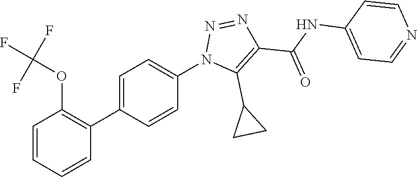 [SMILES: O=C(Nc1ccncc1)c1nnn(-c2ccc(-c3ccccc3OC(F)(F)F)cc2)c1C1CC1] |
| 7-187 | 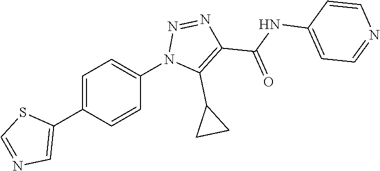 [SMILES: O=C(Nc1ccncc1)c1nnn(-c2ccc(-c3cncs3)cc2)c1C1CC1] |
| 7-188 | 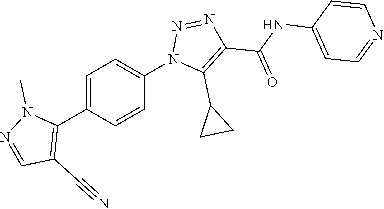 [SMILES: Cn1ncc(C#N)c1-c1ccc(-n2nnc(C(=O)Nc3ccncc3)c2C2CC2)cc1] |
| 7-189 | 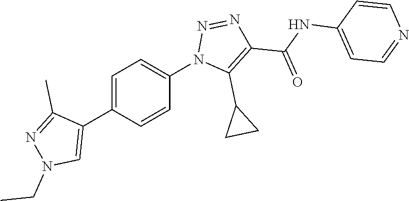 [SMILES: CCn1cc(-c2ccc(-n3nnc(C(=O)Nc4ccncc4)c3C3CC3)cc2)c(C)n1] |
| 7-190 | 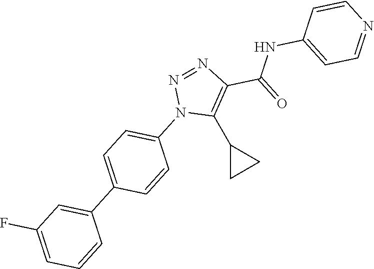 [SMILES: O=C(Nc1ccncc1)c1nnn(-c2ccc(-c3cccc(F)c3)cc2)c1C1CC1] |
| 7-191 | 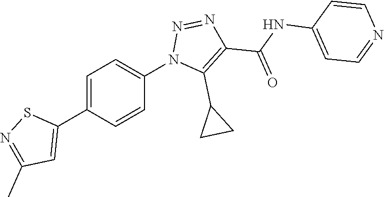 [SMILES: Cc1cc(-c2ccc(-n3nnc(C(=O)Nc4ccncc4)c3C3CC3)cc2)sn1] |
| 7-192 | 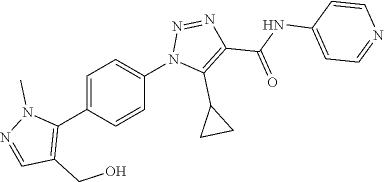 [SMILES: Cn1ncc(CO)c1-c1ccc(-n2nnc(C(=O)Nc3ccncc3)c2C2CC2)cc1] |
| 7-193 | 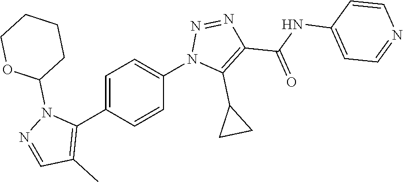 [SMILES: Cc1cnn(C2CCCCO2)c1-c1ccc(-n2nnc(C(=O)Nc3ccncc3)c2C2CC2)cc1] |
| 7-194 | 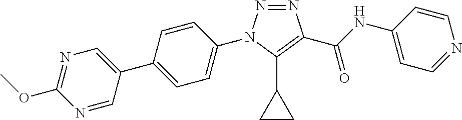 [SMILES: COc1ncc(-c2ccc(-n3nnc(C(=O)Nc4ccncc4)c3C3CC3)cc2)cn1] |
| 7-195 | 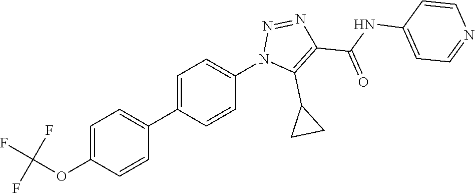 [SMILES: O=C(Nc1ccncc1)c1nnn(-c2ccc(-c3ccc(OC(F)(F)F)cc3)cc2)c1C1CC1] |
| 7-196 | 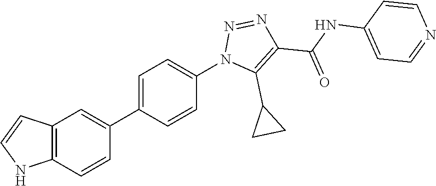 [SMILES: O=C(Nc1ccncc1)c1nnn(-c2ccc(-c3ccc4[nH]ccc4c3)cc2)c1C1CC1] |
| 7-197 | 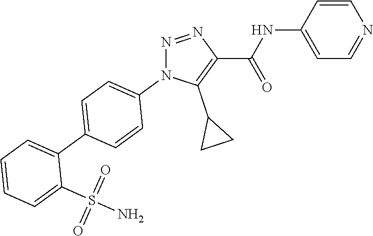 [SMILES: NS(=O)(=O)c1ccccc1-c1ccc(-n2nnc(C(=O)Nc3ccncc3)c2C2CC2)cc1] |
| 7-198 | 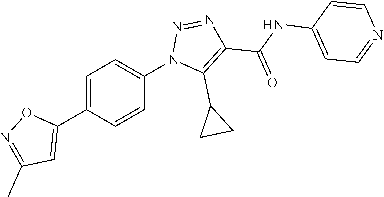 [SMILES: Cc1cc(-c2ccc(-n3nnc(C(=O)Nc4ccncc4)c3C3CC3)cc2)on1] |
| 7-199 | 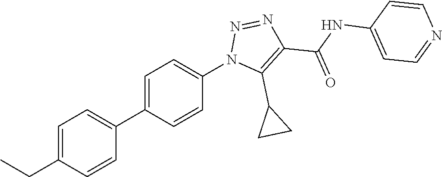 [SMILES: CCc1ccc(-c2ccc(-n3nnc(C(=O)Nc4ccncc4)c3C3CC3)cc2)cc1] |
| 7-200 | 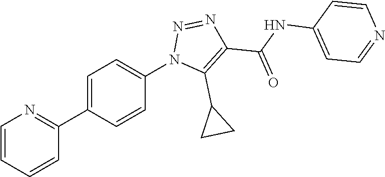 [SMILES: O=C(Nc1ccncc1)c1nnn(-c2ccc(-c3ccccn3)cc2)c1C1CC1] |
| 7-201 | 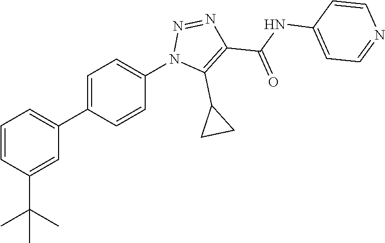 [SMILES: CC(C)(C)c1cccc(-c2ccc(-n3nnc(C(=O)Nc4ccncc4)c3C3CC3)cc2)c1] |
| 7-202 | 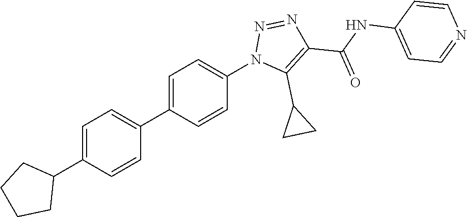 [SMILES: O=C(Nc1ccncc1)c1nnn(-c2ccc(-c3ccc(C4CCCC4)cc3)cc2)c1C1CC1] |
| 7-203 | 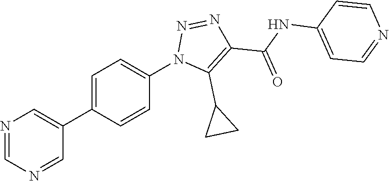 [SMILES: O=C(Nc1ccncc1)c1nnn(-c2ccc(-c3cncnc3)cc2)c1C1CC1] |
| 7-204 | 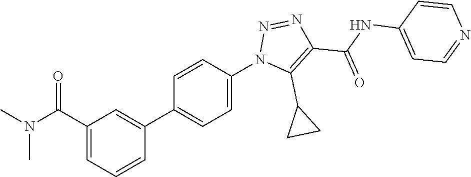 [SMILES: CN(C)C(=O)c1cccc(-c2ccc(-n3nnc(C(=O)Nc4ccncc4)c3C3CC3)cc2)c1] |
| 7-205 | 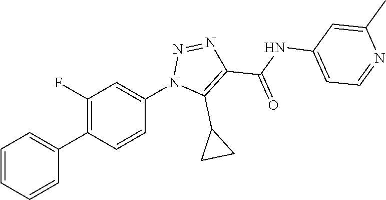 [SMILES: Cc1cc(NC(=O)c2nnn(-c3ccc(-c4ccccc4)c(F)c3)c2C2CC2)ccn1] |
| 7-206 | 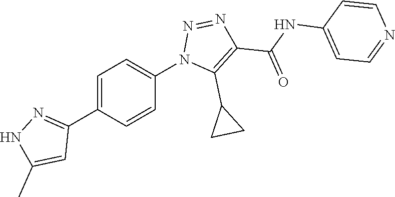 [SMILES: Cc1cc(-c2ccc(-n3nnc(C(=O)Nc4ccncc4)c3C3CC3)cc2)n[nH]1] |
| 7-207 | 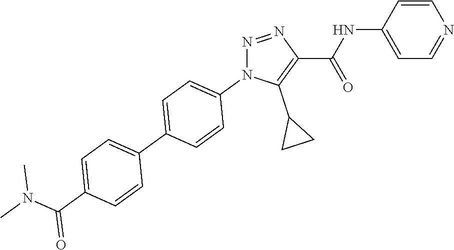 [SMILES: CN(C)C(=O)c1ccc(-c2ccc(-n3nnc(C(=O)Nc4ccncc4)c3C3CC3)cc2)cc1] |
| 7-208 | 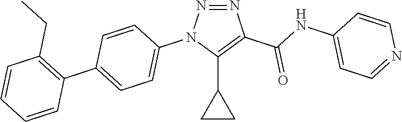 [SMILES: CCc1ccccc1-c1ccc(-n2nnc(C(=O)Nc3ccncc3)c2C2CC2)cc1] |
| 7-209 | 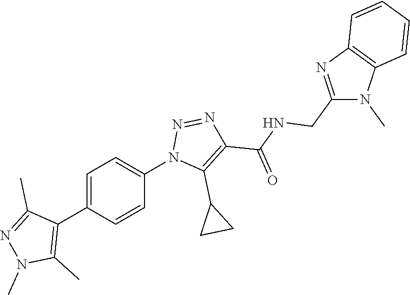 [SMILES: Cc1nn(C)c(C)c1-c1ccc(-n2nnc(C(=O)NCc3nc4ccccc4n3C)c2C2CC2)cc1] |
| 7-210 | 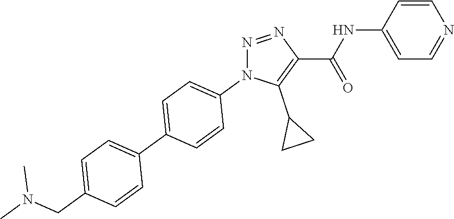 [SMILES: CN(C)Cc1ccc(-c2ccc(-n3nnc(C(=O)Nc4ccncc4)c3C3CC3)cc2)cc1] |
| 7-211 | 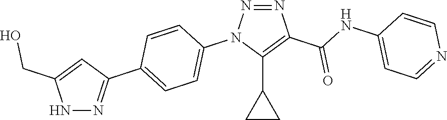 [SMILES: O=C(Nc1ccncc1)c1nnn(-c2ccc(-c3cc(CO)[nH]n3)cc2)c1C1CC1] |
| 7-212 | 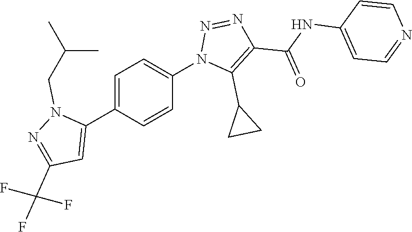 [SMILES: CC(C)Cn1nc(C(F)(F)F)cc1-c1ccc(-n2nnc(C(=O)Nc3ccncc3)c2C2CC2)cc1] |
| 7-213 | 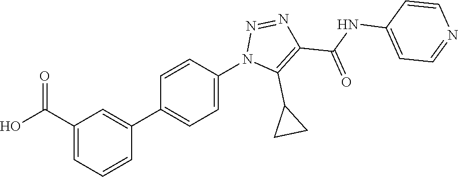 [SMILES: O=C(O)c1cccc(-c2ccc(-n3nnc(C(=O)Nc4ccncc4)c3C3CC3)cc2)c1] |
| 7-214 | 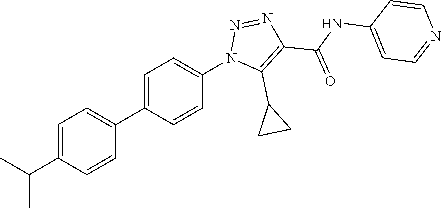 [SMILES: CC(C)c1ccc(-c2ccc(-n3nnc(C(=O)Nc4ccncc4)c3C3CC3)cc2)cc1] |
| 7-215 | 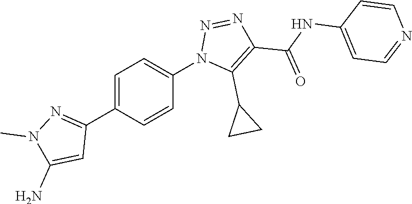 [SMILES: Cn1nc(-c2ccc(-n3nnc(C(=O)Nc4ccncc4)c3C3CC3)cc2)cc1N] |
| 7-216 | 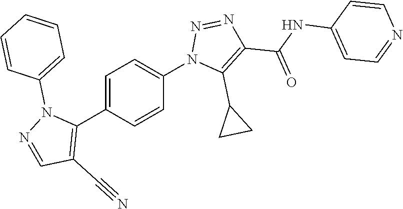 [SMILES: N#Cc1cnn(-c2ccccc2)c1-c1ccc(-n2nnc(C(=O)Nc3ccncc3)c2C2CC2)cc1] |
| 7-217 | 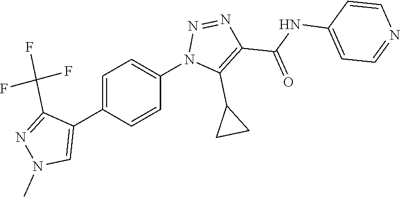 [SMILES: Cn1cc(-c2ccc(-n3nnc(C(=O)Nc4ccncc4)c3C3CC3)cc2)c(C(F)(F)F)n1] |
| 7-218 | 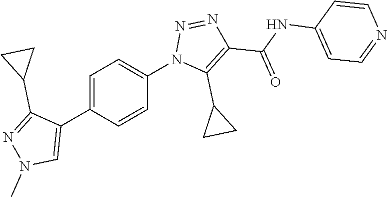 [SMILES: Cn1cc(-c2ccc(-n3nnc(C(=O)Nc4ccncc4)c3C3CC3)cc2)c(C2CC2)n1] |
| 7-219 | 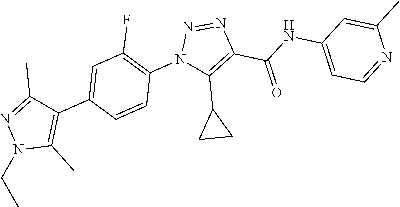 [SMILES: CCn1nc(C)c(-c2ccc(-n3nnc(C(=O)Nc4ccnc(C)c4)c3C3CC3)c(F)c2)c1C] |
| 7-220 | 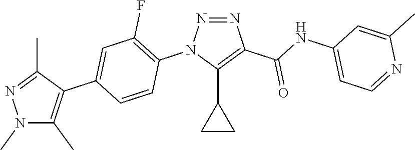 |
| 7-221 | 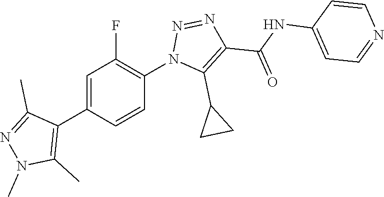 [SMILES: Cc1nn(C)c(C)c1-c1ccc(-n2nnc(C(=O)Nc3ccncc3)c2C2CC2)c(F)c1] |
| 7-222 | 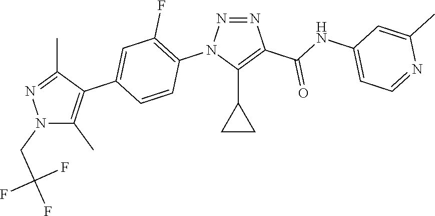 [SMILES: Cc1cc(NC(=O)c2nnn(-c3ccc(-c4c(C)nn(CC(F)(F)F)c4C)cc3F)c2C2CC2)ccn1] |
| 7-223 | 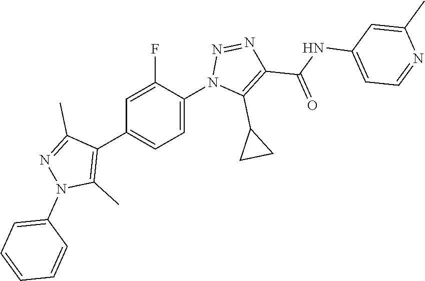 [SMILES: Cc1cc(NC(=O)c2nnn(-c3ccc(-c4c(C)nn(-c5ccccc5)c4C)cc3F)c2C2CC2)ccn1] |
| 7-224 | 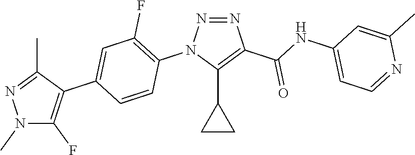 |
| 7-225 | 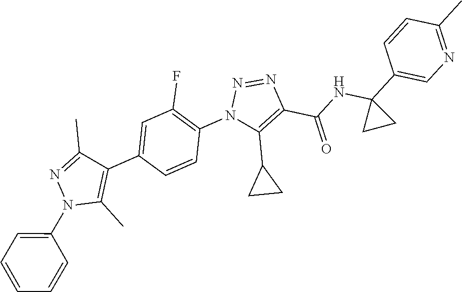 [SMILES: Cc1ccc(C2(NC(=O)c3nnn(-c4ccc(-c5c(C)nn(-c6ccccc6)c5C)cc4F)c3C3CC3)CC2)cn1] |
| 7-226 | 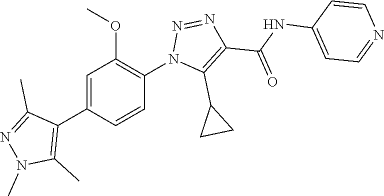 [SMILES: COc1cc(-c2c(C)nn(C)c2C)ccc1-n1nnc(C(=O)Nc2ccncc2)c1C1CC1] |
| 7-227 | 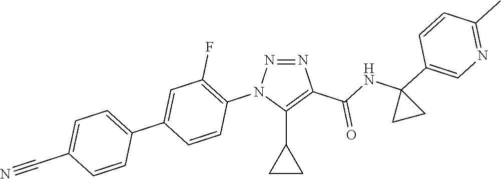 [SMILES: Cc1ccc(C2(NC(=O)c3nnn(-c4ccc(-c5ccc(C#N)cc5)cc4F)c3C3CC3)CC2)cn1] |
| 7-228 | 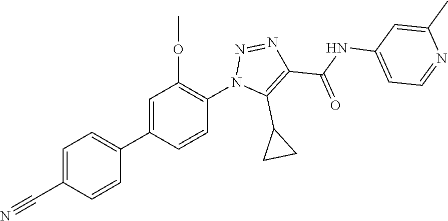 [SMILES: COc1cc(-c2ccc(C#N)cc2)ccc1-n1nnc(C(=O)Nc2ccnc(C)c2)c1C1CC1] |
| 7-229 | 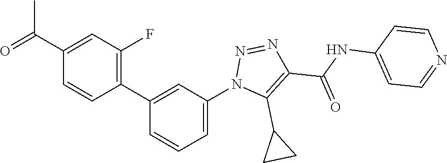 [SMILES: CC(=O)c1ccc(-c2cccc(-n3nnc(C(=O)Nc4ccncc4)c3C3CC3)c2)c(F)c1] |
| 7-230 | 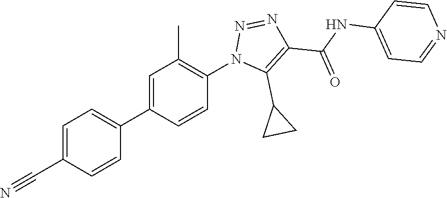 [SMILES: Cc1cc(-c2ccc(C#N)cc2)ccc1-n1nnc(C(=O)Nc2ccncc2)c1C1CC1] |
| 7-231 | 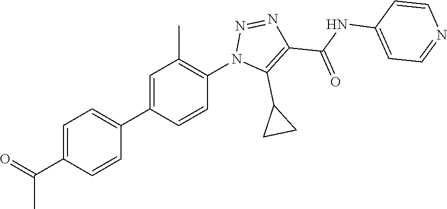 [SMILES: CC(=O)c1ccc(-c2ccc(-n3nnc(C(=O)Nc4ccncc4)c3C3CC3)c(C)c2)cc1] |
| 7-232 | 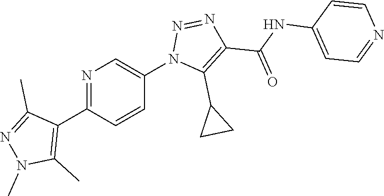 [SMILES: Cc1nn(C)c(C)c1-c1ccc(-n2nnc(C(=O)Nc3ccncc3)c2C2CC2)cn1] |
| 7-233 | 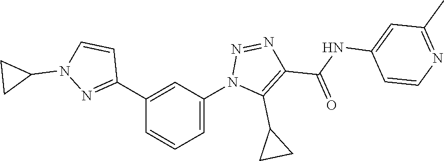 [SMILES: Cc1cc(NC(=O)c2nnn(-c3cccc(-c4ccn(C5CC5)n4)c3)c2C2CC2)ccn1] |
| 7-234 | 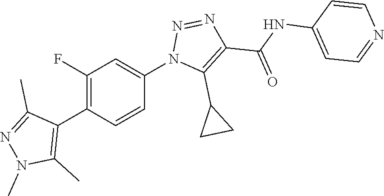 [SMILES: Cc1nn(C)c(C)c1-c1ccc(-n2nnc(C(=O)Nc3ccncc3)c2C2CC2)cc1F] |
| 7-235 | 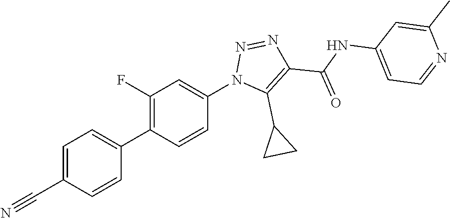 [SMILES: Cc1cc(NC(=O)c2nnn(-c3ccc(-c4ccc(C#N)cc4)c(F)c3)c2C2CC2)ccn1] |
| 7-236 | 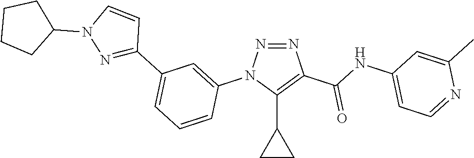 [SMILES: Cc1cc(NC(=O)c2nnn(-c3cccc(-c4ccn(C5CCCC5)n4)c3)c2C2CC2)ccn1] |
| 7-237 | 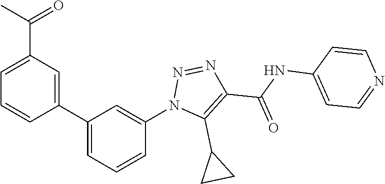 [SMILES: CC(=O)c1cccc(-c2cccc(-n3nnc(C(=O)Nc4ccncc4)c3C3CC3)c2)c1] |
| 7-238 | 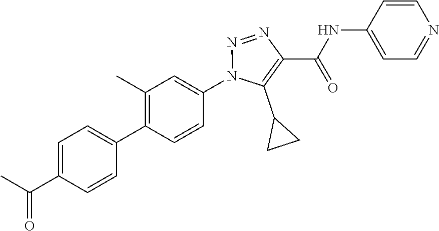 [SMILES: CC(=O)c1ccc(-c2ccc(-n3nnc(C(=O)Nc4ccncc4)c3C3CC3)cc2C)cc1] |
| 7-239 | 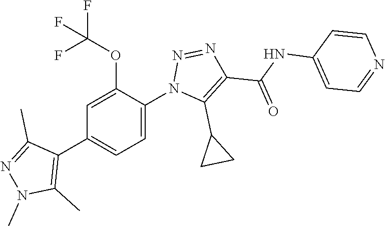 [SMILES: Cc1nn(C)c(C)c1-c1ccc(-n2nnc(C(=O)Nc3ccncc3)c2C2CC2)c(OC(F)(F)F)c1] |
| 7-240 | 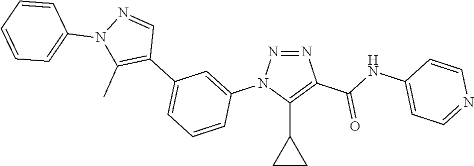 [SMILES: Cc1c(-c2cccc(-n3nnc(C(=O)Nc4ccncc4)c3C3CC3)c2)cnn1-c1ccccc1] |
| 7-241 | 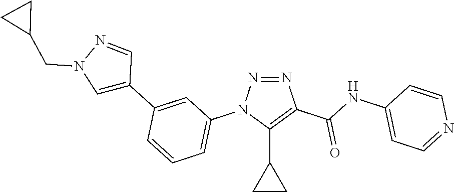 [SMILES: O=C(Nc1ccncc1)c1nnn(-c2cccc(-c3cnn(CC4CC4)c3)c2)c1C1CC1] |
| 7-242 | 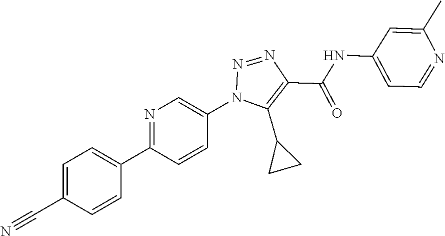 [SMILES: Cc1cc(NC(=O)c2nnn(-c3ccc(-c4ccc(C#N)cc4)nc3)c2C2CC2)ccn1] |
| 7-243 | 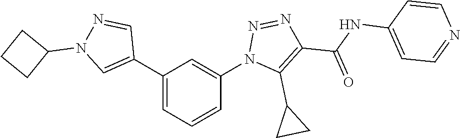 [SMILES: O=C(Nc1ccncc1)c1nnn(-c2cccc(-c3cnn(C4CCC4)c3)c2)c1C1CC1] |
| 7-244 | 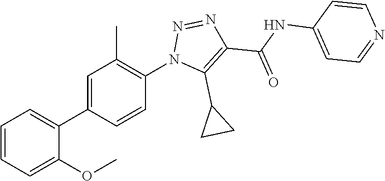 [SMILES: COc1ccccc1-c1ccc(-n2nnc(C(=O)Nc3ccncc3)c2C2CC2)c(C)c1] |
| 7-245 | 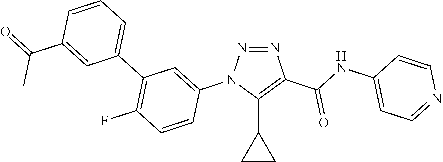 [SMILES: CC(=O)c1cccc(-c2cc(-n3nnc(C(=O)Nc4ccncc4)c3C3CC3)ccc2F)c1] |
| 7-246 | 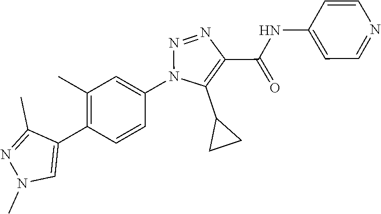 [SMILES: Cc1cc(-n2nnc(C(=O)Nc3ccncc3)c2C2CC2)ccc1-c1cn(C)nc1C] |
| 7-247 | 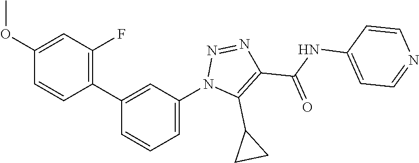 [SMILES: COc1ccc(-c2cccc(-n3nnc(C(=O)Nc4ccncc4)c3C3CC3)c2)c(F)c1] |
| 7-248 | 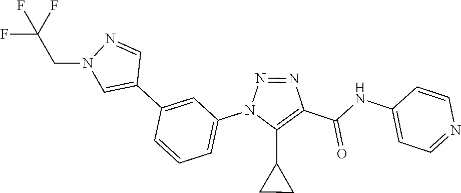 [SMILES: O=C(Nc1ccncc1)c1nnn(-c2cccc(-c3cnn(CC(F)(F)F)c3)c2)c1C1CC1] |
| 7-249 | 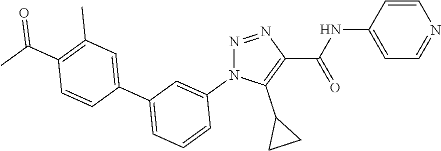 [SMILES: CC(=O)c1ccc(-c2cccc(-n3nnc(C(=O)Nc4ccncc4)c3C3CC3)c2)cc1C] |
| 7-250 | 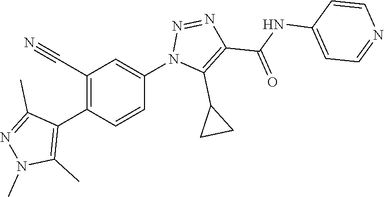 [SMILES: Cc1nn(C)c(C)c1-c1ccc(-n2nnc(C(=O)Nc3ccncc3)c2C2CC2)cc1C#N] |
| 7-251 | 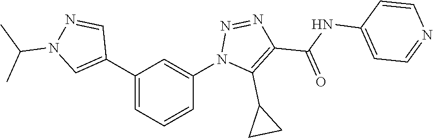 [SMILES: CC(C)n1cc(-c2cccc(-n3nnc(C(=O)Nc4ccncc4)c3C3CC3)c2)cn1] |
| 7-252 | 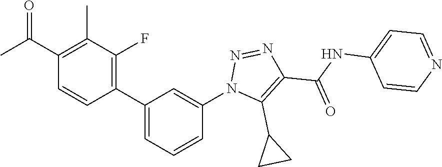 [SMILES: CC(=O)c1ccc(-c2cccc(-n3nnc(C(=O)Nc4ccncc4)c3C3CC3)c2)c(F)c1C] |
| 7-253 | 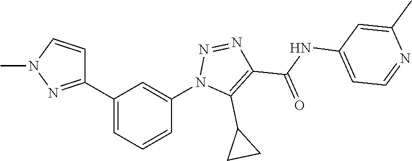 |
| 7-255 | 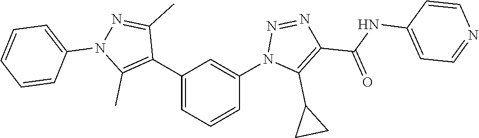 [SMILES: Cc1nn(-c2ccccc2)c(C)c1-c1cccc(-n2nnc(C(=O)Nc3ccncc3)c2C2CC2)c1] |
| 7-256 | 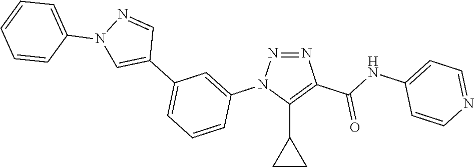 [SMILES: O=C(Nc1ccncc1)c1nnn(-c2cccc(-c3cnn(-c4ccccc4)c3)c2)c1C1CC1] |
| 7-257 | 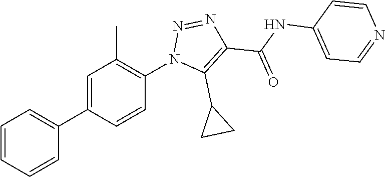 [SMILES: Cc1cc(-c2ccccc2)ccc1-n1nnc(C(=O)Nc2ccncc2)c1C1CC1] |
| 7-258 | 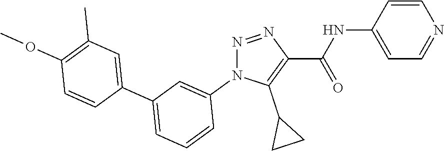 [SMILES: COc1ccc(-c2cccc(-n3nnc(C(=O)Nc4ccncc4)c3C3CC3)c2)cc1C] |
| 7-259 | 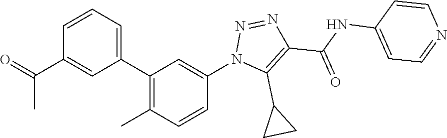 [SMILES: CC(=O)c1cccc(-c2cc(-n3nnc(C(=O)Nc4ccncc4)c3C3CC3)ccc2C)c1] |
| 7-260 | 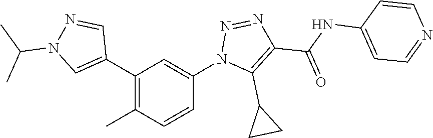 [SMILES: Cc1ccc(-n2nnc(C(=O)Nc3ccncc3)c2C2CC2)cc1-c1cnn(C(C)C)c1] |
| 7-261 | 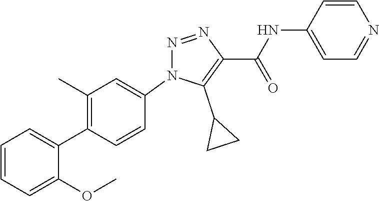 [SMILES: COc1ccccc1-c1ccc(-n2nnc(C(=O)Nc3ccncc3)c2C2CC2)cc1C] |
| 7-262 | 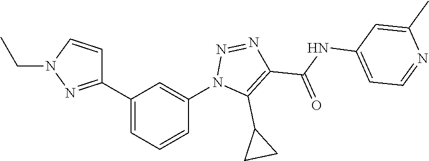 [SMILES: CCn1ccc(-c2cccc(-n3nnc(C(=O)Nc4ccnc(C)c4)c3C3CC3)c2)n1] |
| 7-263 | 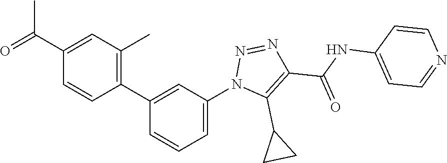 [SMILES: CC(=O)c1ccc(-c2cccc(-n3nnc(C(=O)Nc4ccncc4)c3C3CC3)c2)c(C)c1] |
| 7-264 | 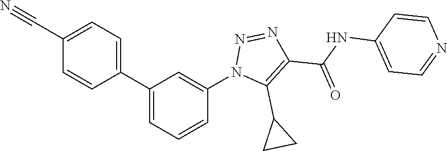 [SMILES: N#Cc1ccc(-c2cccc(-n3nnc(C(=O)Nc4ccncc4)c3C3CC3)c2)cc1] |
| 7-265 | 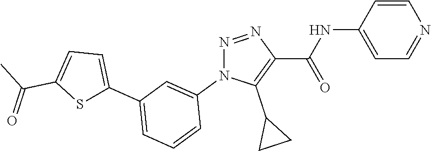 [SMILES: CC(=O)c1ccc(-c2cccc(-n3nnc(C(=O)Nc4ccncc4)c3C3CC3)c2)s1] |
| 7-266 | 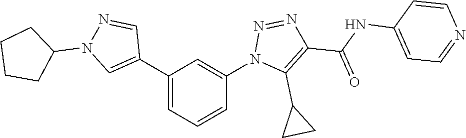 [SMILES: O=C(Nc1ccncc1)c1nnn(-c2cccc(-c3cnn(C4CCCC4)c3)c2)c1C1CC1] |
| 7-267 | 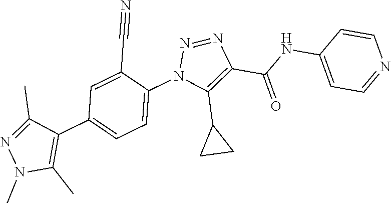 [SMILES: Cc1nn(C)c(C)c1-c1ccc(-n2nnc(C(=O)Nc3ccncc3)c2C2CC2)c(C#N)c1] |
| 7-268 | 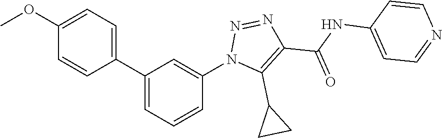 [SMILES: COc1ccc(-c2cccc(-n3nnc(C(=O)Nc4ccncc4)c3C3CC3)c2)cc1] |
| 7-269 | 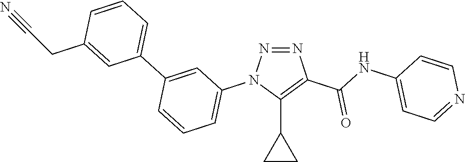 [SMILES: N#CCc1cccc(-c2cccc(-n3nnc(C(=O)Nc4ccncc4)c3C3CC3)c2)c1] |
| 7-270 | 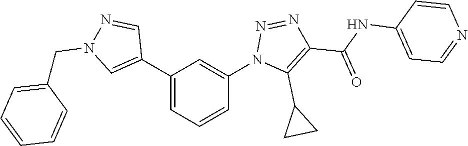 [SMILES: O=C(Nc1ccncc1)c1nnn(-c2cccc(-c3cnn(Cc4ccccc4)c3)c2)c1C1CC1] |
| 7-271 | 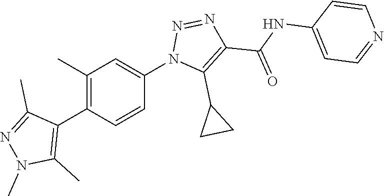 [SMILES: Cc1cc(-n2nnc(C(=O)Nc3ccncc3)c2C2CC2)ccc1-c1c(C)nn(C)c1C] |
| 7-272 | 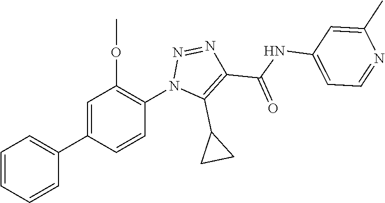 [SMILES: COc1cc(-c2ccccc2)ccc1-n1nnc(C(=O)Nc2ccnc(C)c2)c1C1CC1] |
| 7-273 | 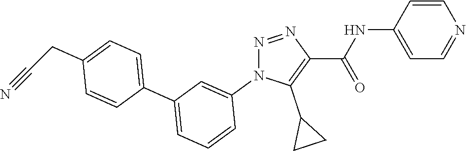 [SMILES: N#CCc1ccc(-c2cccc(-n3nnc(C(=O)Nc4ccncc4)c3C3CC3)c2)cc1] |
| 7-274 | 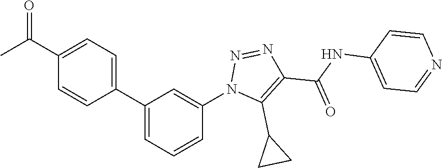 [SMILES: CC(=O)c1ccc(-c2cccc(-n3nnc(C(=O)Nc4ccncc4)c3C3CC3)c2)cc1] |
| 7-275 | 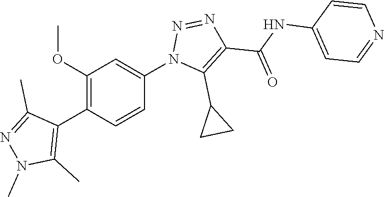 [SMILES: COc1cc(-n2nnc(C(=O)Nc3ccncc3)c2C2CC2)ccc1-c1c(C)nn(C)c1C] |
| 7-276 | 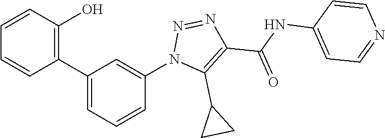 [SMILES: O=C(Nc1ccncc1)c1nnn(-c2cccc(-c3ccccc3O)c2)c1C1CC1] |
| 7-277 | 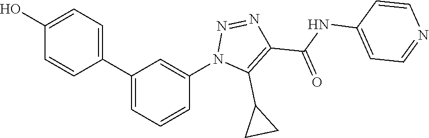 [SMILES: O=C(Nc1ccncc1)c1nnn(-c2cccc(-c3ccc(O)cc3)c2)c1C1CC1] |
| 7-278 | 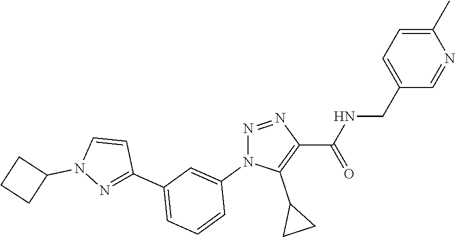 [SMILES: Cc1ccc(CNC(=O)c2nnn(-c3cccc(-c4ccn(C5CCC5)n4)c3)c2C2CC2)cn1] |
| 7-279 | 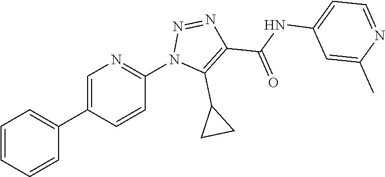 [SMILES: Cc1cc(NC(=O)c2nnn(-c3ccc(-c4ccccc4)cn3)c2C2CC2)ccn1] |
| 7-280 | 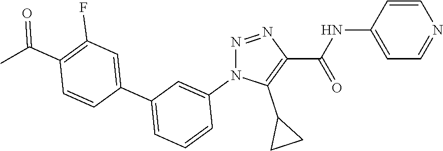 [SMILES: CC(=O)c1ccc(-c2cccc(-n3nnc(C(=O)Nc4ccncc4)c3C3CC3)c2)cc1F] |
| 7-281 | 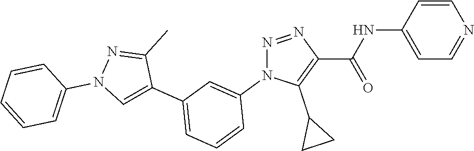 [SMILES: Cc1nn(-c2ccccc2)cc1-c1cccc(-n2nnc(C(=O)Nc3ccncc3)c2C2CC2)c1] |
| 7-282 | 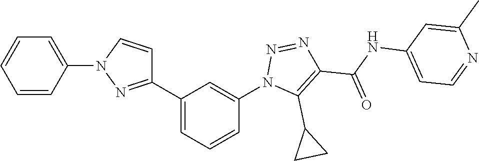 [SMILES: Cc1cc(NC(=O)c2nnn(-c3cccc(-c4ccn(-c5ccccc5)n4)c3)c2C2CC2)ccn1] |
| 7-283 | 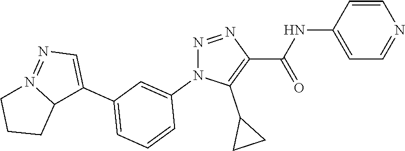 |
| 7-284 | 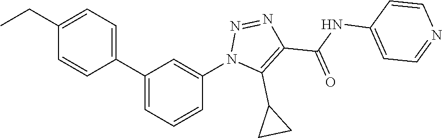 [SMILES: CCc1ccc(-c2cccc(-n3nnc(C(=O)Nc4ccncc4)c3C3CC3)c2)cc1] |
| 7-285 | 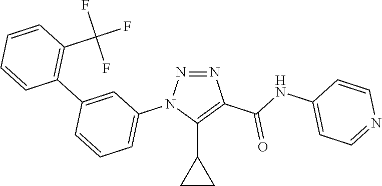 [SMILES: O=C(Nc1ccncc1)c1nnn(-c2cccc(-c3ccccc3C(F)(F)F)c2)c1C1CC1] |
| 7-286 | 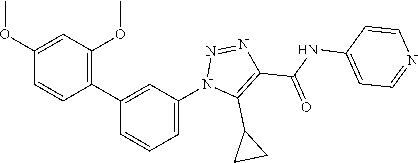 [SMILES: COc1ccc(-c2cccc(-n3nnc(C(=O)Nc4ccncc4)c3C3CC3)c2)c(OC)c1] |
| 7-287 | 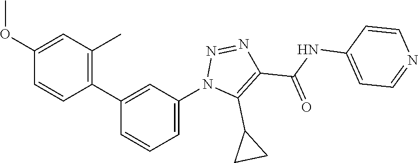 [SMILES: COc1ccc(-c2cccc(-n3nnc(C(=O)Nc4ccncc4)c3C3CC3)c2)c(C)c1] |
| 7-288 | 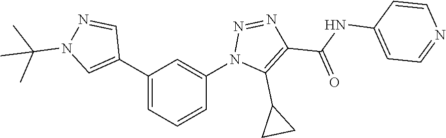 [SMILES: CC(C)(C)n1cc(-c2cccc(-n3nnc(C(=O)Nc4ccncc4)c3C3CC3)c2)cn1] |
| 7-289 | 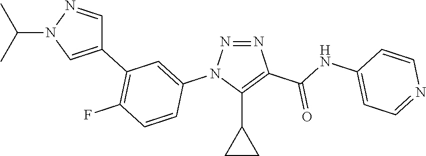 [SMILES: CC(C)n1cc(-c2cc(-n3nnc(C(=O)Nc4ccncc4)c3C3CC3)ccc2F)cn1] |
| 7-290 | 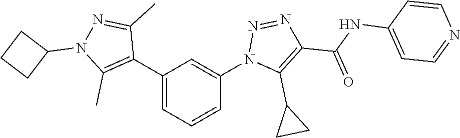 [SMILES: Cc1nn(C2CCC2)c(C)c1-c1cccc(-n2nnc(C(=O)Nc3ccncc3)c2C2CC2)c1] |
| 7-291 | 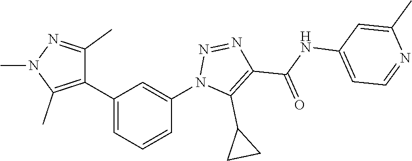 |
| 7-292 | 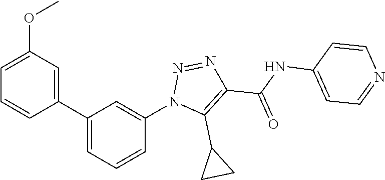 [SMILES: COc1cccc(-c2cccc(-n3nnc(C(=O)Nc4ccncc4)c3C3CC3)c2)c1] |
| 7-293 | 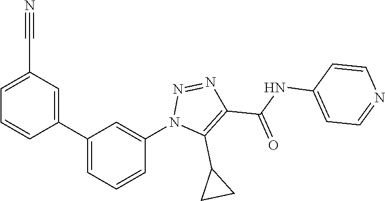 [SMILES: N#Cc1cccc(-c2cccc(-n3nnc(C(=O)Nc4ccncc4)c3C3CC3)c2)c1] |
| 7-294 | 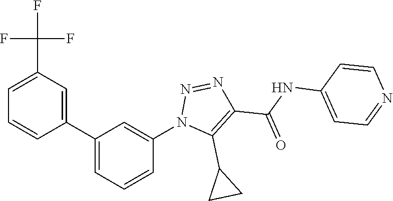 [SMILES: O=C(Nc1ccncc1)c1nnn(-c2cccc(-c3cccc(C(F)(F)F)c3)c2)c1C1CC1] |
| 7-295 | 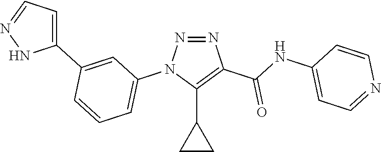 [SMILES: O=C(Nc1ccncc1)c1nnn(-c2cccc(-c3ccn[nH]3)c2)c1C1CC1] |
| 7-296 | 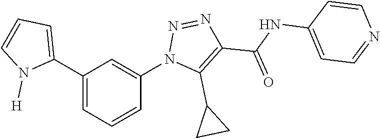 [SMILES: O=C(Nc1ccncc1)c1nnn(-c2cccc(-c3ccc[nH]3)c2)c1C1CC1] |
| 7-297 | 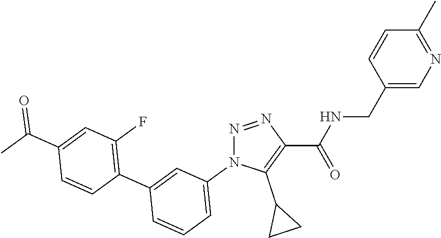 [SMILES: CC(=O)c1ccc(-c2cccc(-n3nnc(C(=O)NCc4ccc(C)nc4)c3C3CC3)c2)c(F)c1] |
| 7-298 | 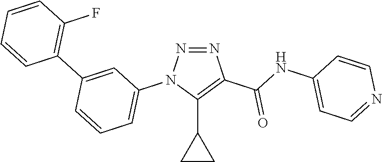 [SMILES: O=C(Nc1ccncc1)c1nnn(-c2cccc(-c3ccccc3F)c2)c1C1CC1] |
| 7-299 | 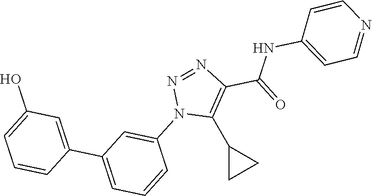 [SMILES: O=C(Nc1ccncc1)c1nnn(-c2cccc(-c3cccc(O)c3)c2)c1C1CC1] |
| 7-300 | 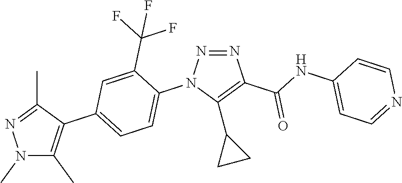 [SMILES: Cc1nn(C)c(C)c1-c1ccc(-n2nnc(C(=O)Nc3ccncc3)c2C2CC2)c(C(F)(F)F)c1] |
| 7-301 | 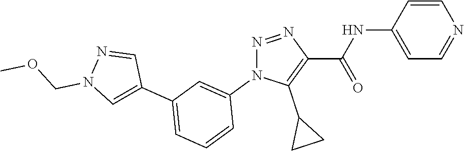 [SMILES: COCn1cc(-c2cccc(-n3nnc(C(=O)Nc4ccncc4)c3C3CC3)c2)cn1] |
| 7-302 | 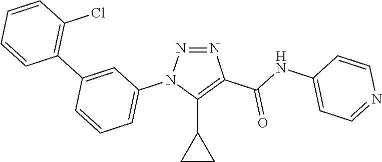 [SMILES: O=C(Nc1ccncc1)c1nnn(-c2cccc(-c3ccccc3Cl)c2)c1C1CC1] |
| 7-303 | 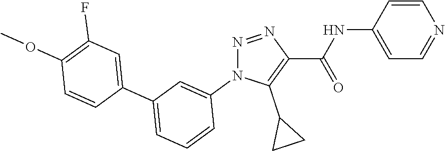 [SMILES: COc1ccc(-c2cccc(-n3nnc(C(=O)Nc4ccncc4)c3C3CC3)c2)cc1F] |
| 7-304 | 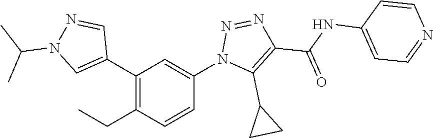 [SMILES: CCc1ccc(-n2nnc(C(=O)Nc3ccncc3)c2C2CC2)cc1-c1cnn(C(C)C)c1] |
| 7-305 | 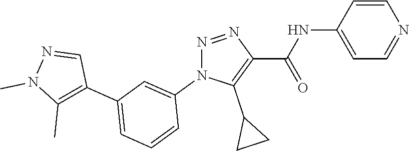 [SMILES: Cc1c(-c2cccc(-n3nnc(C(=O)Nc4ccncc4)c3C3CC3)c2)cnn1C] |
| 7-306 | 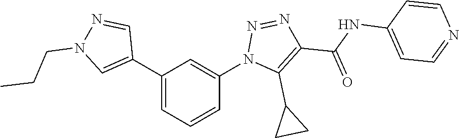 [SMILES: CCCn1cc(-c2cccc(-n3nnc(C(=O)Nc4ccncc4)c3C3CC3)c2)cn1] |
| 7-307 | 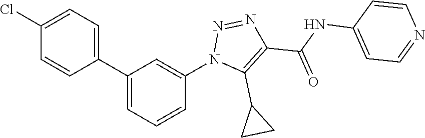 [SMILES: O=C(Nc1ccncc1)c1nnn(-c2cccc(-c3ccc(Cl)cc3)c2)c1C1CC1] |
| 7-308 | 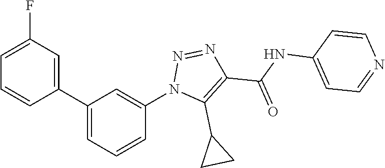 [SMILES: O=C(Nc1ccncc1)c1nnn(-c2cccc(-c3cccc(F)c3)c2)c1C1CC1] |
| 7-309 | 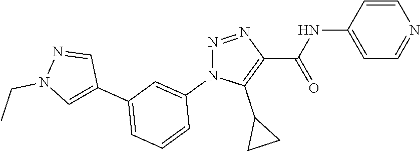 [SMILES: CCn1cc(-c2cccc(-n3nnc(C(=O)Nc4ccncc4)c3C3CC3)c2)cn1] |
| 7-310 | 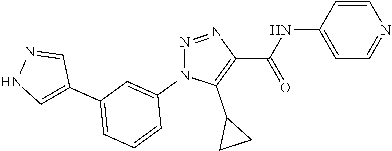 [SMILES: O=C(Nc1ccncc1)c1nnn(-c2cccc(-c3cn[nH]c3)c2)c1C1CC1] |
| 7-311 | 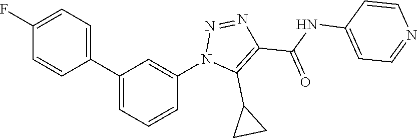 [SMILES: O=C(Nc1ccncc1)c1nnn(-c2cccc(-c3ccc(F)cc3)c2)c1C1CC1] |
| 7-312 | 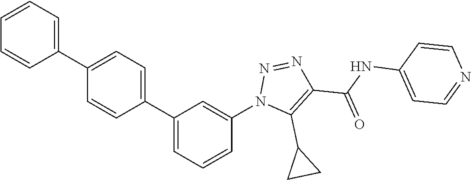 [SMILES: O=C(Nc1ccncc1)c1nnn(-c2cccc(-c3ccc(-c4ccccc4)cc3)c2)c1C1CC1] |
| 7-313 | 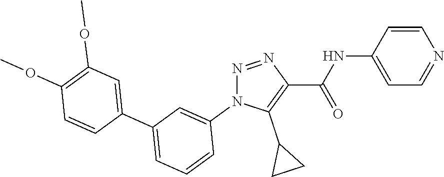 [SMILES: COc1ccc(-c2cccc(-n3nnc(C(=O)Nc4ccncc4)c3C3CC3)c2)cc1OC] |
| 7-314 | 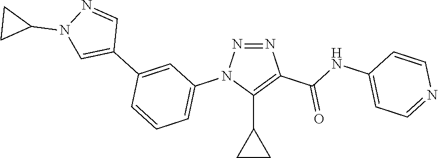 [SMILES: O=C(Nc1ccncc1)c1nnn(-c2cccc(-c3cnn(C4CC4)c3)c2)c1C1CC1] |
| 7-315 | 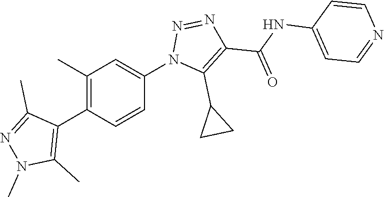 [SMILES: Cc1cc(-n2nnc(C(=O)Nc3ccncc3)c2C2CC2)ccc1-c1c(C)nn(C)c1C] |
| 7-316 | 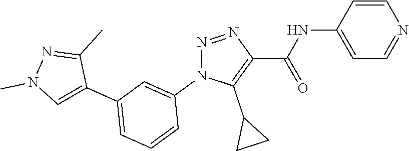 [SMILES: Cc1nn(C)cc1-c1cccc(-n2nnc(C(=O)Nc3ccncc3)c2C2CC2)c1] |
| 7-317 | 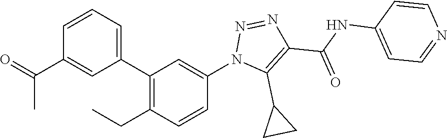 [SMILES: CCc1ccc(-n2nnc(C(=O)Nc3ccncc3)c2C2CC2)cc1-c1cccc(C(C)=O)c1] |
| 7-318 | 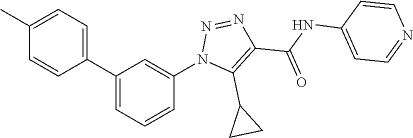 [SMILES: Cc1ccc(-c2cccc(-n3nnc(C(=O)Nc4ccncc4)c3C3CC3)c2)cc1] |
| 7-319 | 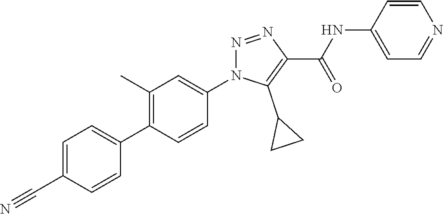 [SMILES: Cc1cc(-n2nnc(C(=O)Nc3ccncc3)c2C2CC2)ccc1-c1ccc(C#N)cc1] |
| 7-320 | 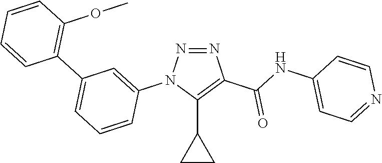 [SMILES: COc1ccccc1-c1cccc(-n2nnc(C(=O)Nc3ccncc3)c2C2CC2)c1] |
| 7-321 | 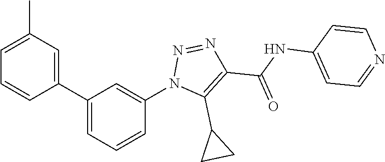 [SMILES: Cc1cccc(-c2cccc(-n3nnc(C(=O)Nc4ccncc4)c3C3CC3)c2)c1] |
| 7-322 | 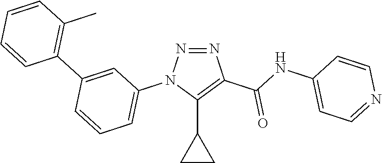 [SMILES: Cc1ccccc1-c1cccc(-n2nnc(C(=O)Nc3ccncc3)c2C2CC2)c1] |
| 7-323 | 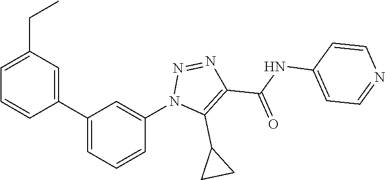 [SMILES: CCc1cccc(-c2cccc(-n3nnc(C(=O)Nc4ccncc4)c3C3CC3)c2)c1] |
| 7-324 | 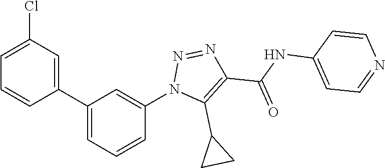 [SMILES: O=C(Nc1ccncc1)c1nnn(-c2cccc(-c3cccc(Cl)c3)c2)c1C1CC1] |
| 7-325 | 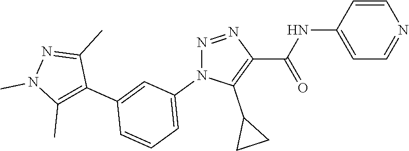 [SMILES: Cc1nn(C)c(C)c1-c1cccc(-n2nnc(C(=O)Nc3ccncc3)c2C2CC2)c1] |
| 7-326 | 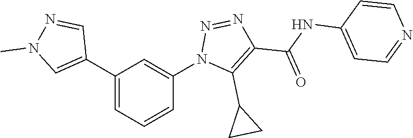 [SMILES: Cn1cc(-c2cccc(-n3nnc(C(=O)Nc4ccncc4)c3C3CC3)c2)cn1] |
| 7-327 | 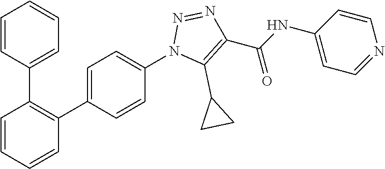 [SMILES: O=C(Nc1ccncc1)c1nnn(-c2ccc(-c3ccccc3-c3ccccc3)cc2)c1C1CC1] |
| 7-328 | 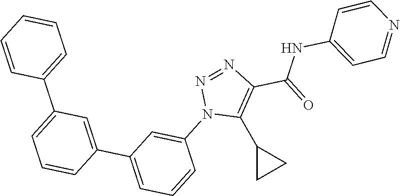 [SMILES: O=C(Nc1ccncc1)c1nnn(-c2cccc(-c3cccc(-c4ccccc4)c3)c2)c1C1CC1] |
| 7-329 | 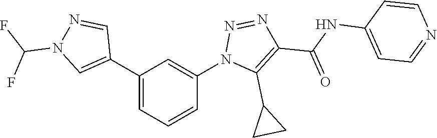 [SMILES: O=C(Nc1ccncc1)c1nnn(-c2cccc(-c3cnn(C(F)F)c3)c2)c1C1CC1] |
| 7-330 | 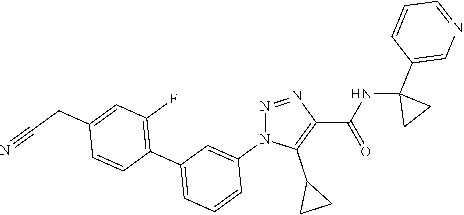 [SMILES: N#CCc1ccc(-c2cccc(-n3nnc(C(=O)NC4(c5cccnc5)CC4)c3C3CC3)c2)c(F)c1] |
| 7-331 | 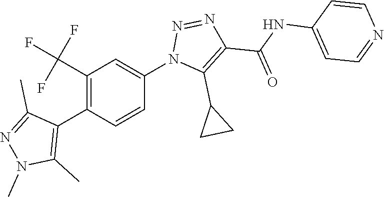 [SMILES: Cc1nn(C)c(C)c1-c1ccc(-n2nnc(C(=O)Nc3ccncc3)c2C2CC2)cc1C(F)(F)F] |
| 7-332 | 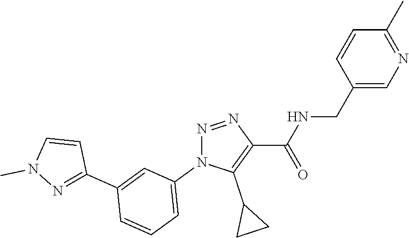 |
| 7-333 | 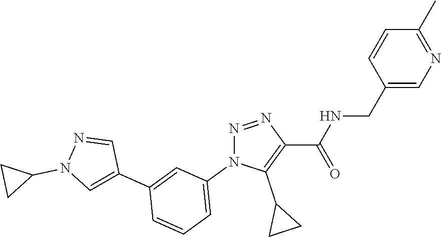 [SMILES: Cc1ccc(CNC(=O)c2nnn(-c3cccc(-c4cnn(C5CC5)c4)c3)c2C2CC2)cn1] |
| 7-334 | 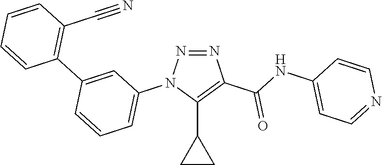 [SMILES: N#Cc1ccccc1-c1cccc(-n2nnc(C(=O)Nc3ccncc3)c2C2CC2)c1] |
| 7-335 | 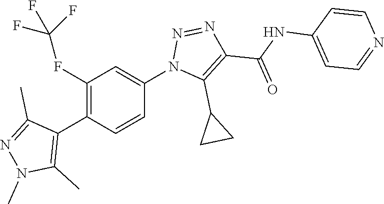 |
| 8-1 | 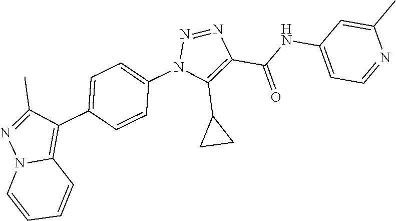 [SMILES: Cc1cc(NC(=O)c2nnn(-c3ccc(-c4c(C)nn5ccccc45)cc3)c2C2CC2)ccn1] |
| 8-2 | 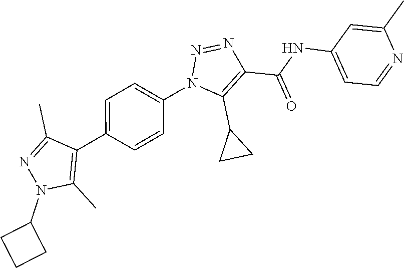 [SMILES: Cc1cc(NC(=O)c2nnn(-c3ccc(-c4c(C)nn(C5CCC5)c4C)cc3)c2C2CC2)ccn1] |
| 8-3 | 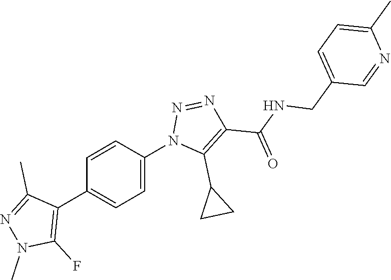 [SMILES: Cc1ccc(CNC(=O)c2nnn(-c3ccc(-c4c(C)nn(C)c4F)cc3)c2C2CC2)cn1] |
| 8-4 | 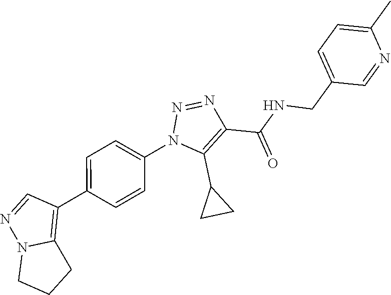 [SMILES: Cc1ccc(CNC(=O)c2nnn(-c3ccc(-c4cnn5c4CCC5)cc3)c2C2CC2)cn1] |
| 9-1 | 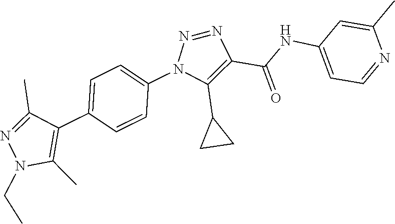 [SMILES: CCn1nc(C)c(-c2ccc(-n3nnc(C(=O)Nc4ccnc(C)c4)c3C3CC3)cc2)c1C] |
| 9-2 | 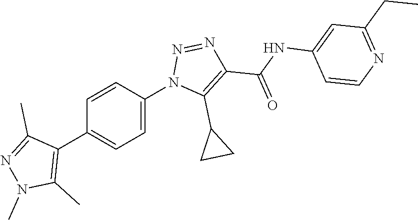 [SMILES: CCc1cc(NC(=O)c2nnn(-c3ccc(-c4c(C)nn(C)c4C)cc3)c2C2CC2)ccn1] |
| 9-3 | 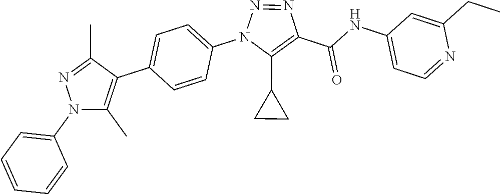 [SMILES: CCc1cc(NC(=O)c2nnn(-c3ccc(-c4c(C)nn(-c5ccccc5)c4C)cc3)c2C2CC2)ccn1] |
| 9-4 | 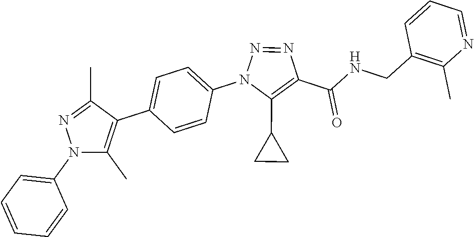 [SMILES: Cc1ncccc1CNC(=O)c1nnn(-c2ccc(-c3c(C)nn(-c4ccccc4)c3C)cc2)c1C1CC1] |
| 9-5 | 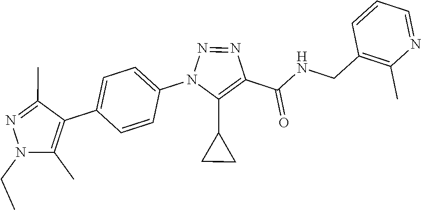 [SMILES: CCn1nc(C)c(-c2ccc(-n3nnc(C(=O)NCc4cccnc4C)c3C3CC3)cc2)c1C] |
| 9-6 | 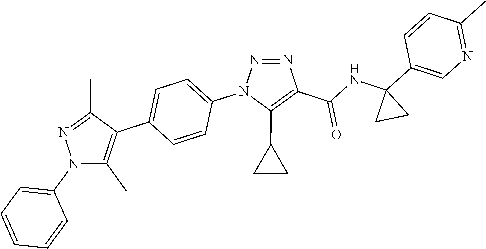 [SMILES: Cc1ccc(C2(NC(=O)c3nnn(-c4ccc(-c5c(C)nn(-c6ccccc6)c5C)cc4)c3C3CC3)CC2)cn1] |
| 9-7 | 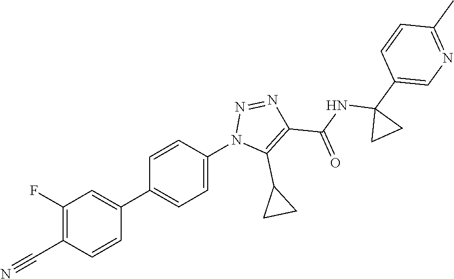 [SMILES: Cc1ccc(C2(NC(=O)c3nnn(-c4ccc(-c5ccc(C#N)c(F)c5)cc4)c3C3CC3)CC2)cn1] |
| 9-8 | 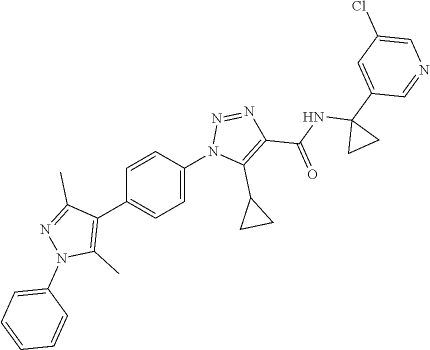 [SMILES: Cc1nn(-c2ccccc2)c(C)c1-c1ccc(-n2nnc(C(=O)NC3(c4cncc(Cl)c4)CC3)c2C2CC2)cc1] |
| 9-9 | 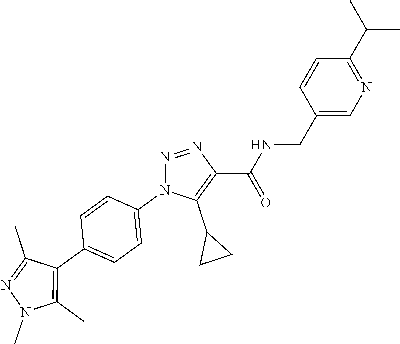 [SMILES: Cc1nn(C)c(C)c1-c1ccc(-n2nnc(C(=O)NCc3ccc(C(C)C)nc3)c2C2CC2)cc1] |
| 9-10 | 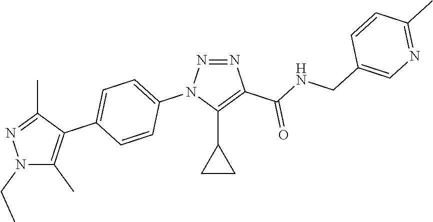 [SMILES: CCn1nc(C)c(-c2ccc(-n3nnc(C(=O)NCc4ccc(C)nc4)c3C3CC3)cc2)c1C] |
| 9-11 | 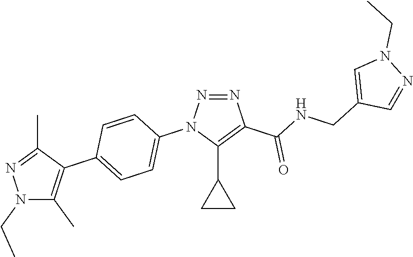 [SMILES: CCn1cc(CNC(=O)c2nnn(-c3ccc(-c4c(C)nn(CC)c4C)cc3)c2C2CC2)cn1] |
| 9-12 | 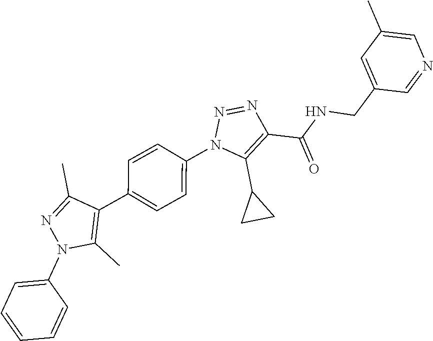 [SMILES: Cc1cncc(CNC(=O)c2nnn(-c3ccc(-c4c(C)nn(-c5ccccc5)c4C)cc3)c2C2CC2)c1] |
| 9-13 | 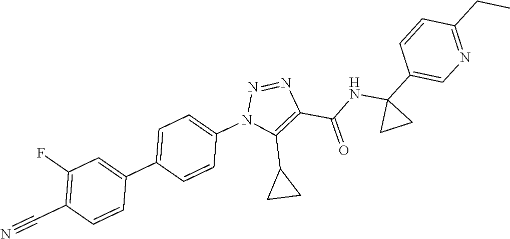 [SMILES: CCc1ccc(C2(NC(=O)c3nnn(-c4ccc(-c5ccc(C#N)c(F)c5)cc4)c3C3CC3)CC2)cn1] |
| 9-14 | 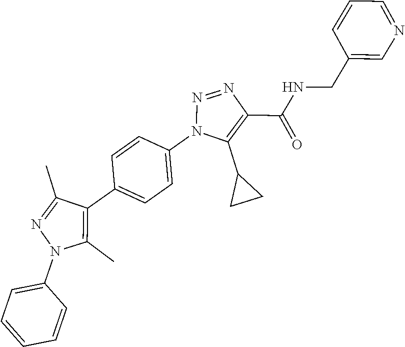 [SMILES: Cc1nn(-c2ccccc2)c(C)c1-c1ccc(-n2nnc(C(=O)NCc3cccnc3)c2C2CC2)cc1] |
| 9-15 | 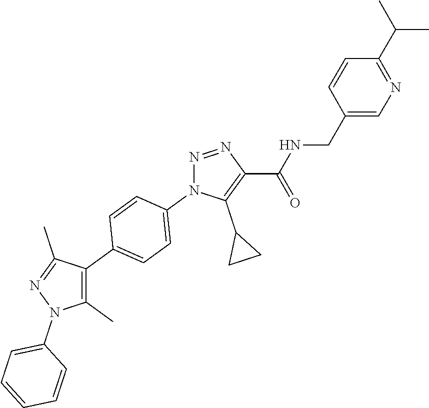 [SMILES: Cc1nn(-c2ccccc2)c(C)c1-c1ccc(-n2nnc(C(=O)NCc3ccc(C(C)C)nc3)c2C2CC2)cc1] |
| 9-16 | 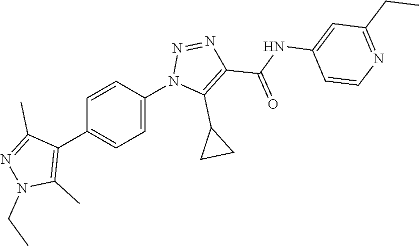 [SMILES: CCc1cc(NC(=O)c2nnn(-c3ccc(-c4c(C)nn(CC)c4C)cc3)c2C2CC2)ccn1] |
| 9-17 | 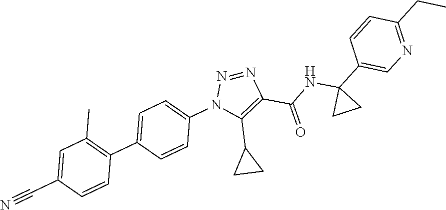 [SMILES: CCc1ccc(C2(NC(=O)c3nnn(-c4ccc(-c5ccc(C#N)cc5C)cc4)c3C3CC3)CC2)cn1] |
| 9-18 | 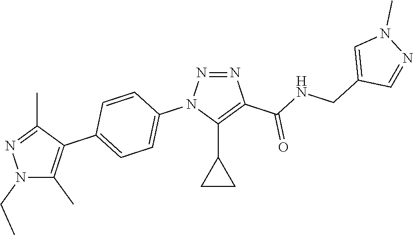 [SMILES: CCn1nc(C)c(-c2ccc(-n3nnc(C(=O)NCc4cnn(C)c4)c3C3CC3)cc2)c1C] |
| 9-19 | 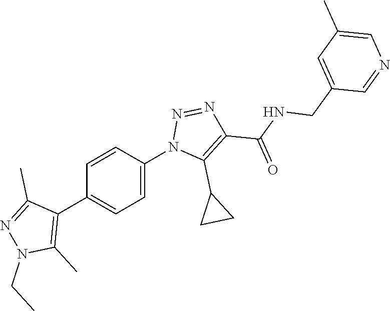 [SMILES: CCn1nc(C)c(-c2ccc(-n3nnc(C(=O)NCc4cncc(C)c4)c3C3CC3)cc2)c1C] |
| 9-20 | 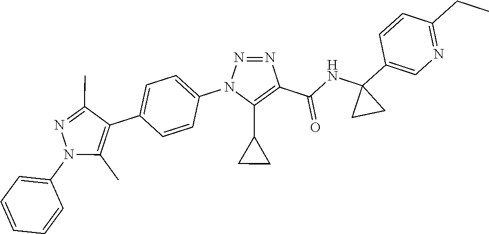 [SMILES: CCc1ccc(C2(NC(=O)c3nnn(-c4ccc(-c5c(C)nn(-c6ccccc6)c5C)cc4)c3C3CC3)CC2)cn1] |
| 9-21 | 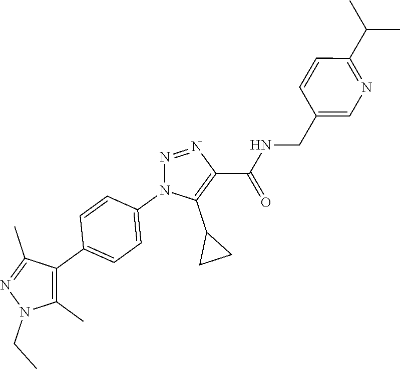 [SMILES: CCn1nc(C)c(-c2ccc(-n3nnc(C(=O)NCc4ccc(C(C)C)nc4)c3C3CC3)cc2)c1C] |
| 9-22 | 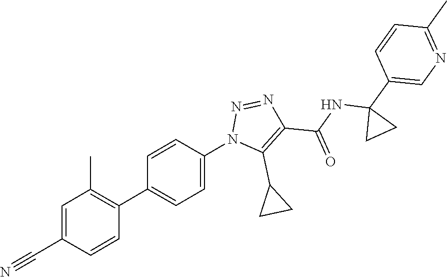 [SMILES: Cc1ccc(C2(NC(=O)c3nnn(-c4ccc(-c5ccc(C#N)cc5C)cc4)c3C3CC3)CC2)cn1] |
| 9-23 | 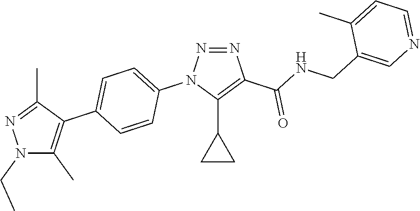 [SMILES: CCn1nc(C)c(-c2ccc(-n3nnc(C(=O)NCc4cnccc4C)c3C3CC3)cc2)c1C] |
| 9-24 | 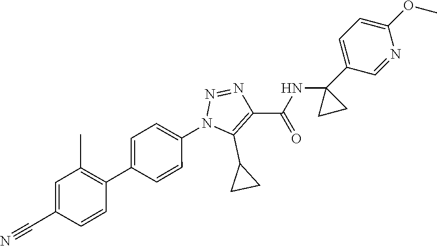 [SMILES: COc1ccc(C2(NC(=O)c3nnn(-c4ccc(-c5ccc(C#N)cc5C)cc4)c3C3CC3)CC2)cn1] |
| 9-25 | 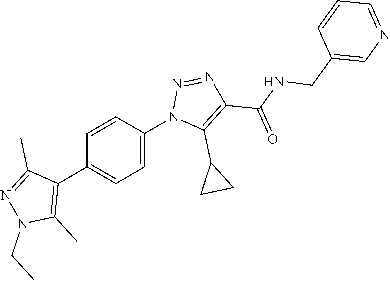 [SMILES: CCn1nc(C)c(-c2ccc(-n3nnc(C(=O)NCc4cccnc4)c3C3CC3)cc2)c1C] |
| 9-26 | 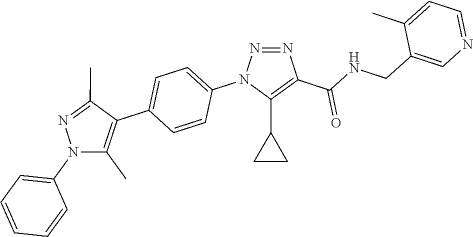 [SMILES: Cc1ccncc1CNC(=O)c1nnn(-c2ccc(-c3c(C)nn(-c4ccccc4)c3C)cc2)c1C1CC1] |
| 9-27 | 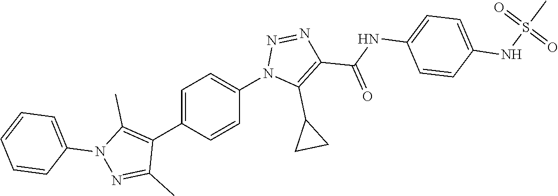 [SMILES: Cc1nn(-c2ccccc2)c(C)c1-c1ccc(-n2nnc(C(=O)Nc3ccc(NS(C)(=O)=O)cc3)c2C2CC2)cc1] |
| 9-28 | 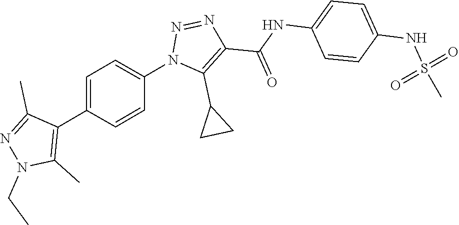 [SMILES: CCn1nc(C)c(-c2ccc(-n3nnc(C(=O)Nc4ccc(NS(C)(=O)=O)cc4)c3C3CC3)cc2)c1C] |
| 9-29 | 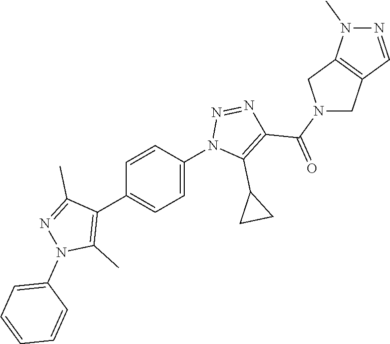 [SMILES: Cc1nn(-c2ccccc2)c(C)c1-c1ccc(-n2nnc(C(=O)N3Cc4cnn(C)c4C3)c2C2CC2)cc1] |
| 9-30 | 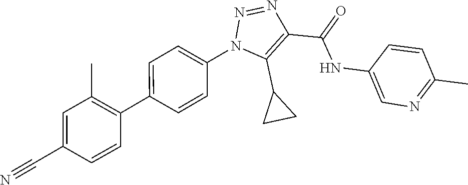 [SMILES: Cc1ccc(NC(=O)c2nnn(-c3ccc(-c4ccc(C#N)cc4C)cc3)c2C2CC2)cn1] |
| 9-31 | 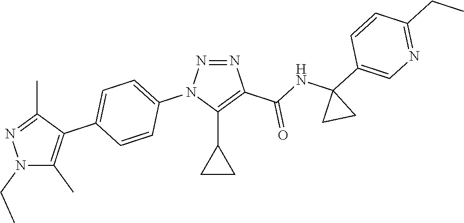 [SMILES: CCc1ccc(C2(NC(=O)c3nnn(-c4ccc(-c5c(C)nn(CC)c5C)cc4)c3C3CC3)CC2)cn1] |
| 9-32 | 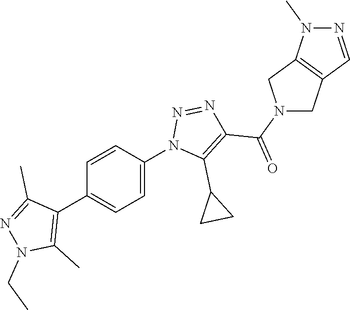 [SMILES: CCn1nc(C)c(-c2ccc(-n3nnc(C(=O)N4Cc5cnn(C)c5C4)c3C3CC3)cc2)c1C] |
| 9-33 | 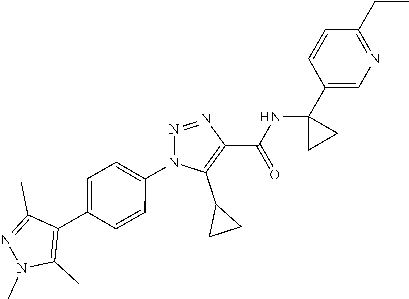 [SMILES: CCc1ccc(C2(NC(=O)c3nnn(-c4ccc(-c5c(C)nn(C)c5C)cc4)c3C3CC3)CC2)cn1] |
| 9-34 | 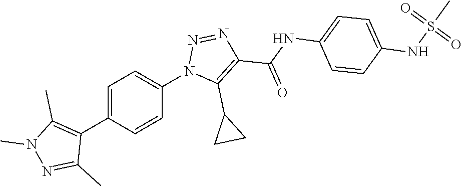 [SMILES: Cc1nn(C)c(C)c1-c1ccc(-n2nnc(C(=O)Nc3ccc(NS(C)(=O)=O)cc3)c2C2CC2)cc1] |
| 9-35 | 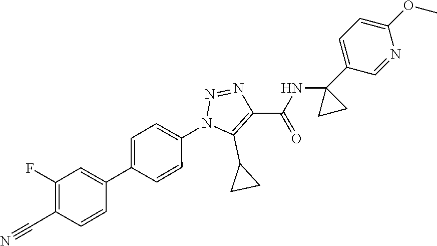 [SMILES: COc1ccc(C2(NC(=O)c3nnn(-c4ccc(-c5ccc(C#N)c(F)c5)cc4)c3C3CC3)CC2)cn1] |
| 9-36 | 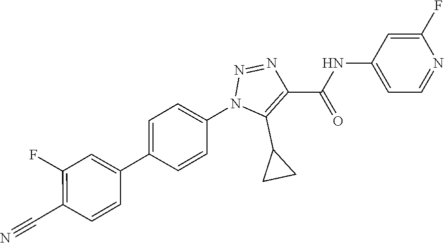 [SMILES: N#Cc1ccc(-c2ccc(-n3nnc(C(=O)Nc4ccnc(F)c4)c3C3CC3)cc2)cc1F] |
| 9-37 | 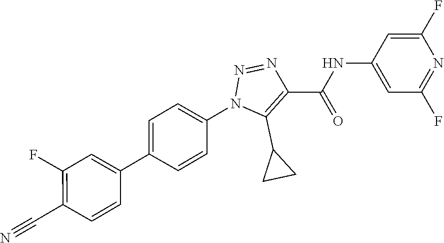 [SMILES: N#Cc1ccc(-c2ccc(-n3nnc(C(=O)Nc4cc(F)nc(F)c4)c3C3CC3)cc2)cc1F] |
| 10-1 | 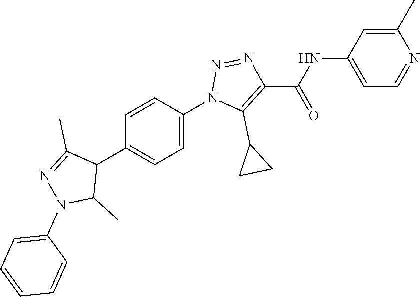 [SMILES: CC1=NN(c2ccccc2)C(C)C1c1ccc(-n2nnc(C(=O)Nc3ccnc(C)c3)c2C2CC2)cc1] |
| 10-2 | 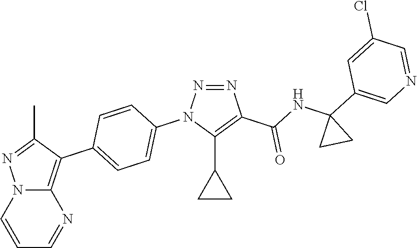 [SMILES: Cc1nn2cccnc2c1-c1ccc(-n2nnc(C(=O)NC3(c4cncc(Cl)c4)CC3)c2C2CC2)cc1] |
| 10-3 | 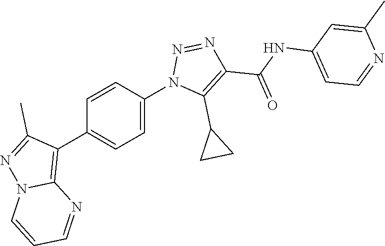 [SMILES: Cc1cc(NC(=O)c2nnn(-c3ccc(-c4c(C)nn5cccnc45)cc3)c2C2CC2)ccn1] |
| 10-4 | 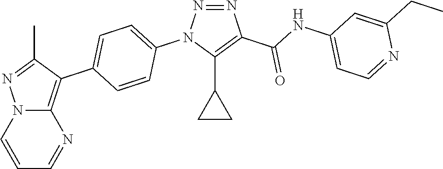 [SMILES: CCc1cc(NC(=O)c2nnn(-c3ccc(-c4c(C)nn5cccnc45)cc3)c2C2CC2)ccn1] |
| 10-5 | 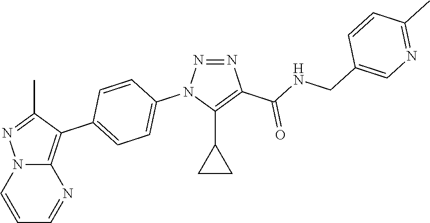 [SMILES: Cc1ccc(CNC(=O)c2nnn(-c3ccc(-c4c(C)nn5cccnc45)cc3)c2C2CC2)cn1] |
| 10-6 | 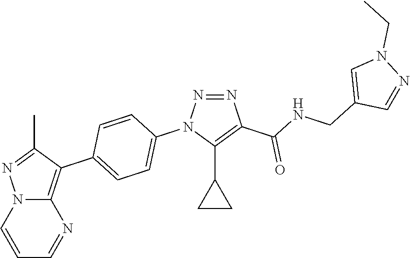 [SMILES: CCn1cc(CNC(=O)c2nnn(-c3ccc(-c4c(C)nn5cccnc45)cc3)c2C2CC2)cn1] |
| 10-7 | 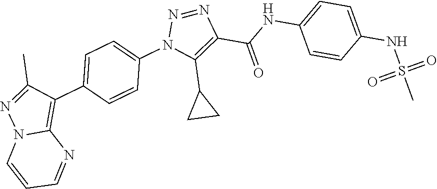 [SMILES: Cc1nn2cccnc2c1-c1ccc(-n2nnc(C(=O)Nc3ccc(NS(C)(=O)=O)cc3)c2C2CC2)cc1] |
| 10-8 | 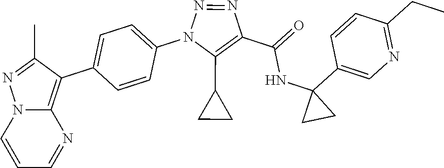 [SMILES: CCc1ccc(C2(NC(=O)c3nnn(-c4ccc(-c5c(C)nn6cccnc56)cc4)c3C3CC3)CC2)cn1] |
| 10-9 | 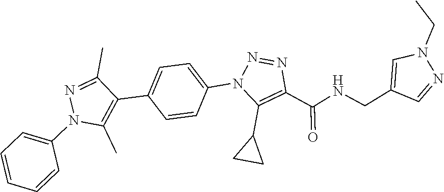 [SMILES: CCn1cc(CNC(=O)c2nnn(-c3ccc(-c4c(C)nn(-c5ccccc5)c4C)cc3)c2C2CC2)cn1] |
| 10-10 | 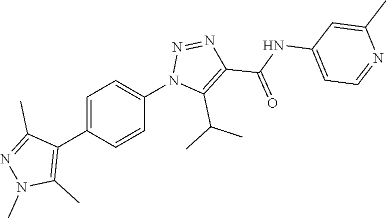 [SMILES: Cc1cc(NC(=O)c2nnn(-c3ccc(-c4c(C)nn(C)c4C)cc3)c2C(C)C)ccn1] |
| 10-11 | 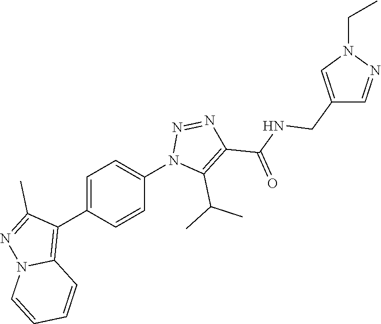 [SMILES: CCn1cc(CNC(=O)c2nnn(-c3ccc(-c4c(C)nn5ccccc45)cc3)c2C(C)C)cn1] |
| 10-12 | 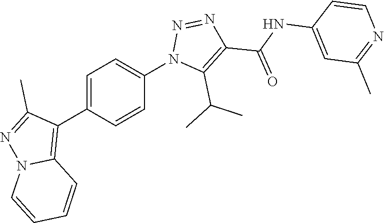 [SMILES: Cc1cc(NC(=O)c2nnn(-c3ccc(-c4c(C)nn5ccccc45)cc3)c2C(C)C)ccn1] |
| 11-1 | 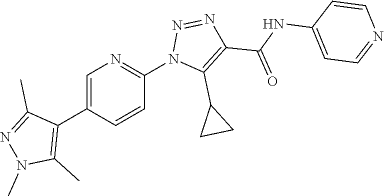 [SMILES: Cc1nn(C)c(C)c1-c1ccc(-n2nnc(C(=O)Nc3ccncc3)c2C2CC2)nc1] |
| 11-2 | 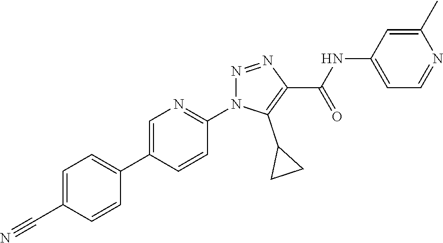 [SMILES: Cc1cc(NC(=O)c2nnn(-c3ccc(-c4ccc(C#N)cc4)cn3)c2C2CC2)ccn1] |
| 12-1 | 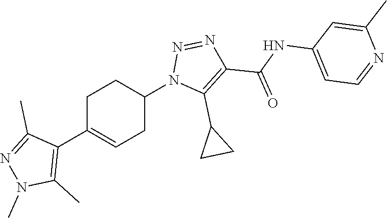 [SMILES: Cc1cc(NC(=O)c2nnn(C3CC=C(c4c(C)nn(C)c4C)CC3)c2C2CC2)ccn1] |
| 12-2 | 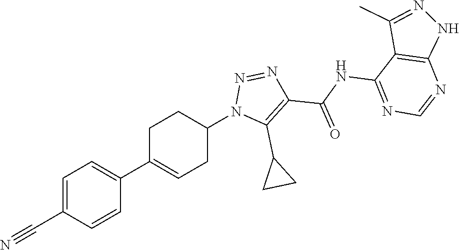 [SMILES: Cc1n[nH]c2ncnc(NC(=O)c3nnn(C4CC=C(c5ccc(C#N)cc5)CC4)c3C3CC3)c12] |
| 12-3 | 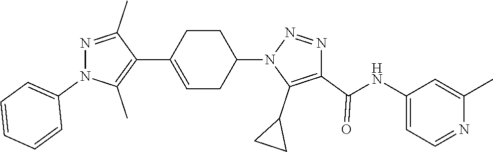 [SMILES: Cc1cc(NC(=O)c2nnn(C3CC=C(c4c(C)nn(-c5ccccc5)c4C)CC3)c2C2CC2)ccn1] |
| 12-4 | 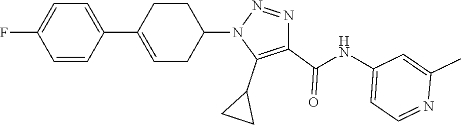 [SMILES: Cc1cc(NC(=O)c2nnn(C3CC=C(c4ccc(F)cc4)CC3)c2C2CC2)ccn1] |
| 12-5 | 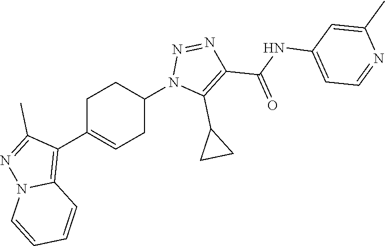 [SMILES: Cc1cc(NC(=O)c2nnn(C3CC=C(c4c(C)nn5ccccc45)CC3)c2C2CC2)ccn1] |
| 12-6 | 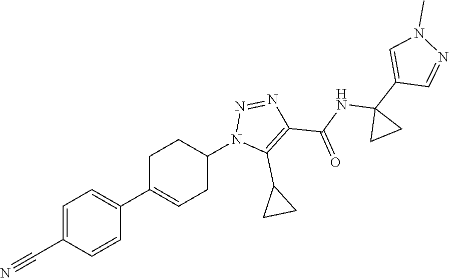 [SMILES: Cn1cc(C2(NC(=O)c3nnn(C4CC=C(c5ccc(C#N)cc5)CC4)c3C3CC3)CC2)cn1] |
| 12-7 | 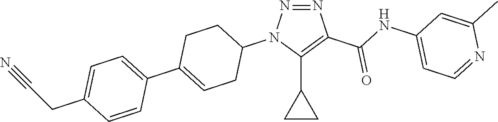 [SMILES: Cc1cc(NC(=O)c2nnn(C3CC=C(c4ccc(CC#N)cc4)CC3)c2C2CC2)ccn1] |
| 12-8 | 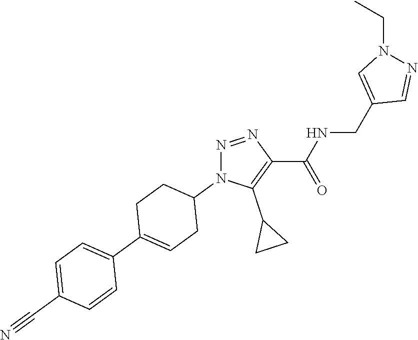 [SMILES: CCn1cc(CNC(=O)c2nnn(C3CC=C(c4ccc(C#N)cc4)CC3)c2C2CC2)cn1] |
| 12-9 | 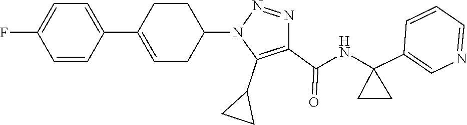 [SMILES: O=C(NC1(c2cccnc2)CC1)c1nnn(C2CC=C(c3ccc(F)cc3)CC2)c1C1CC1] |
| 13-1 | 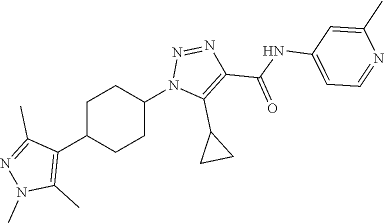 [SMILES: Cc1cc(NC(=O)c2nnn(C3CCC(c4c(C)nn(C)c4C)CC3)c2C2CC2)ccn1] |
| 14-1 | 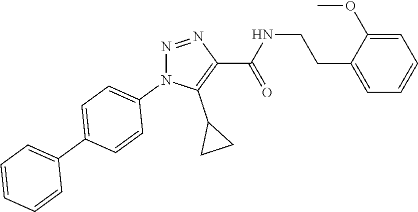 [SMILES: COc1ccccc1CCNC(=O)c1nnn(-c2ccc(-c3ccccc3)cc2)c1C1CC1] |
| 14-2 | 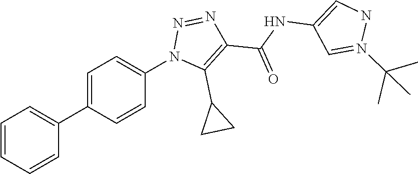 |
| 14-3 | 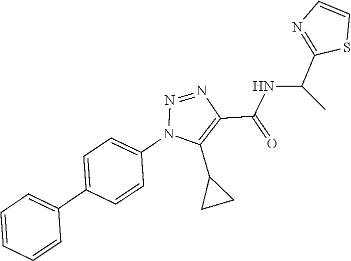 [SMILES: CC(NC(=O)c1nnn(-c2ccc(-c3ccccc3)cc2)c1C1CC1)c1nccs1] |
| 14-4 | 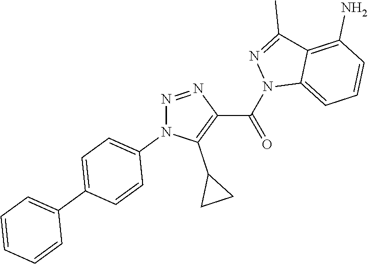 [SMILES: Cc1nn(C(=O)c2nnn(-c3ccc(-c4ccccc4)cc3)c2C2CC2)c2cccc(N)c12] |
| 14-5 | 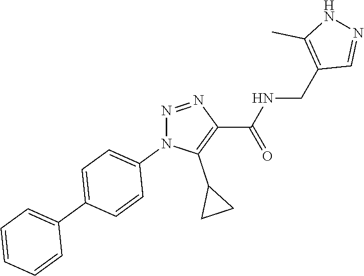 [SMILES: Cc1[nH]ncc1CNC(=O)c1nnn(-c2ccc(-c3ccccc3)cc2)c1C1CC1] |
| 14-6 | 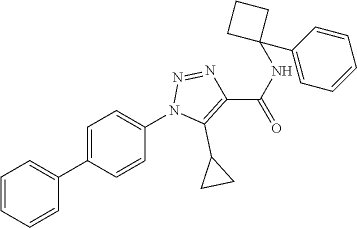 [SMILES: O=C(NC1(c2ccccc2)CCC1)c1nnn(-c2ccc(-c3ccccc3)cc2)c1C1CC1] |
| 14-7 | 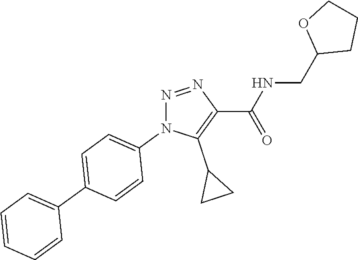 [SMILES: O=C(NCC1CCCO1)c1nnn(-c2ccc(-c3ccccc3)cc2)c1C1CC1] |
| 14-8 | 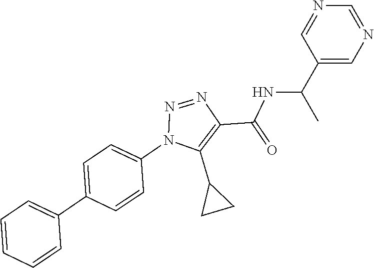 [SMILES: CC(NC(=O)c1nnn(-c2ccc(-c3ccccc3)cc2)c1C1CC1)c1cncnc1] |
| 14-9 | 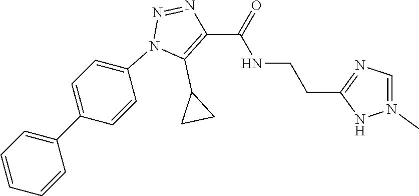 |
| 14-10 | 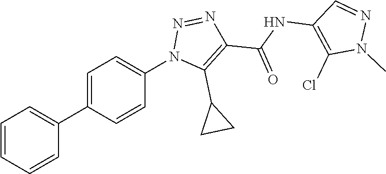 [SMILES: Cn1ncc(NC(=O)c2nnn(-c3ccc(-c4ccccc4)cc3)c2C2CC2)c1Cl] |
| 14-11 | 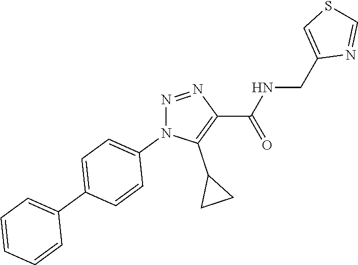 [SMILES: O=C(NCc1cscn1)c1nnn(-c2ccc(-c3ccccc3)cc2)c1C1CC1] |
| 14-12 | 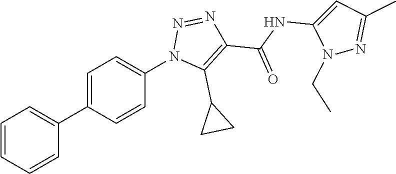 [SMILES: CCn1nc(C)cc1NC(=O)c1nnn(-c2ccc(-c3ccccc3)cc2)c1C1CC1] |
| 14-13 | 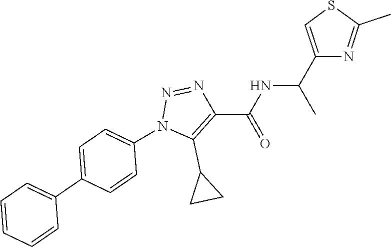 [SMILES: Cc1nc(C(C)NC(=O)c2nnn(-c3ccc(-c4ccccc4)cc3)c2C2CC2)cs1] |
| 14-14 | 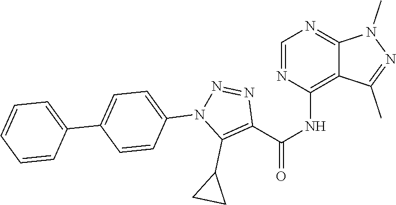 [SMILES: Cc1nn(C)c2ncnc(NC(=O)c3nnn(-c4ccc(-c5ccccc5)cc4)c3C3CC3)c12] |
| 14-15 | 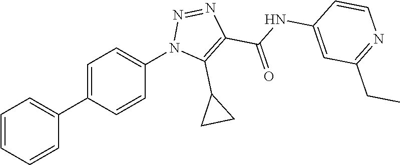 [SMILES: CCc1cc(NC(=O)c2nnn(-c3ccc(-c4ccccc4)cc3)c2C2CC2)ccn1] |
| 14-16 | 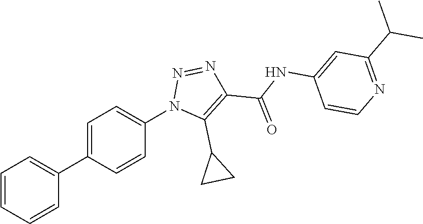 [SMILES: CC(C)c1cc(NC(=O)c2nnn(-c3ccc(-c4ccccc4)cc3)c2C2CC2)ccn1] |
| 14-17 | 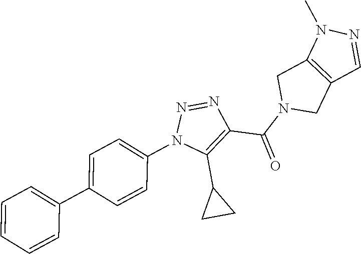 [SMILES: Cn1ncc2c1CN(C(=O)c1nnn(-c3ccc(-c4ccccc4)cc3)c1C1CC1)C2] |
| 14-18 | 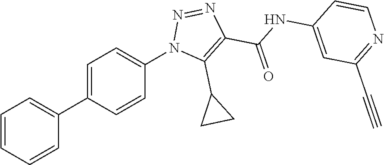 [SMILES: C#Cc1cc(NC(=O)c2nnn(-c3ccc(-c4ccccc4)cc3)c2C2CC2)ccn1] |
| 14-19 | 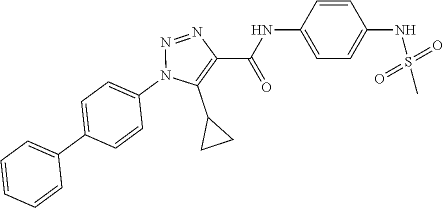 [SMILES: CS(=O)(=O)Nc1ccc(NC(=O)c2nnn(-c3ccc(-c4ccccc4)cc3)c2C2CC2)cc1] |
| 14-20 | 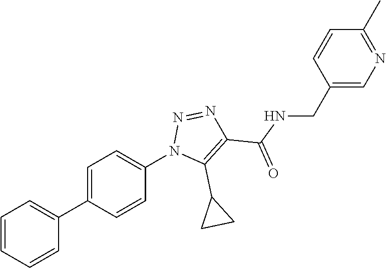 [SMILES: Cc1ccc(CNC(=O)c2nnn(-c3ccc(-c4ccccc4)cc3)c2C2CC2)cn1] |
| 14-21 | 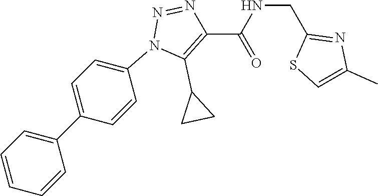 [SMILES: Cc1csc(CNC(=O)c2nnn(-c3ccc(-c4ccccc4)cc3)c2C2CC2)n1] |
| 14-22 | 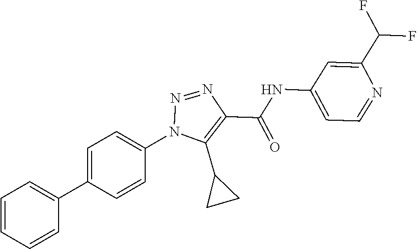 [SMILES: O=C(Nc1ccnc(C(F)F)c1)c1nnn(-c2ccc(-c3ccccc3)cc2)c1C1CC1] |
| 14-23 | 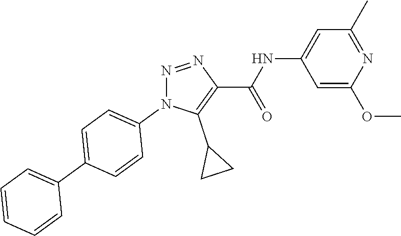 [SMILES: COc1cc(NC(=O)c2nnn(-c3ccc(-c4ccccc4)cc3)c2C2CC2)cc(C)n1] |
| 14-24 | 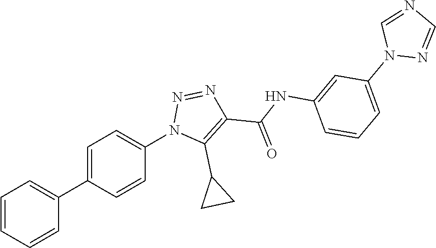 [SMILES: O=C(Nc1cccc(-n2cncn2)c1)c1nnn(-c2ccc(-c3ccccc3)cc2)c1C1CC1] |
| 14-25 | 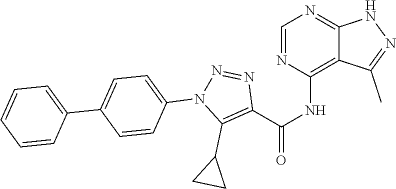 [SMILES: Cc1n[nH]c2ncnc(NC(=O)c3nnn(-c4ccc(-c5ccccc5)cc4)c3C3CC3)c12] |
| 14-26 | 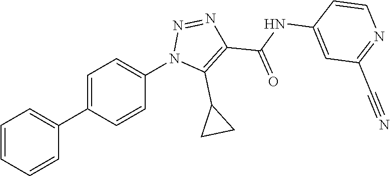 [SMILES: N#Cc1cc(NC(=O)c2nnn(-c3ccc(-c4ccccc4)cc3)c2C2CC2)ccn1] |
| 14-27 | 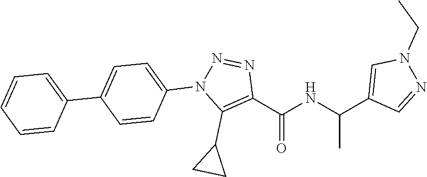 [SMILES: CCn1cc(C(C)NC(=O)c2nnn(-c3ccc(-c4ccccc4)cc3)c2C2CC2)cn1] |
| 14-28 | 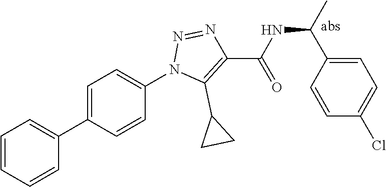 [SMILES: C[C@H](NC(=O)c1nnn(-c2ccc(-c3ccccc3)cc2)c1C1CC1)c1ccc(Cl)cc1] |
| 14-29 | 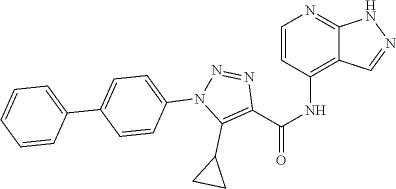 [SMILES: O=C(Nc1ccnc2[nH]ncc12)c1nnn(-c2ccc(-c3ccccc3)cc2)c1C1CC1] |
| 14-30 | 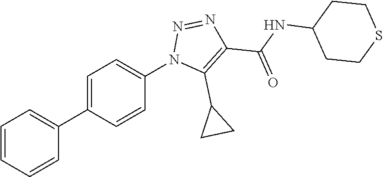 [SMILES: O=C(NC1CCSCC1)c1nnn(-c2ccc(-c3ccccc3)cc2)c1C1CC1] |
| 14-31 | 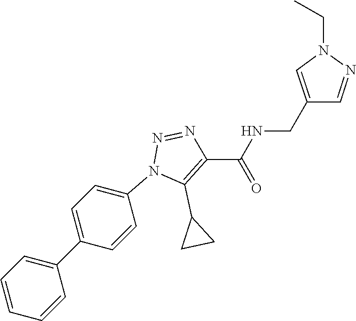 [SMILES: CCn1cc(CNC(=O)c2nnn(-c3ccc(-c4ccccc4)cc3)c2C2CC2)cn1] |
| 14-32 | 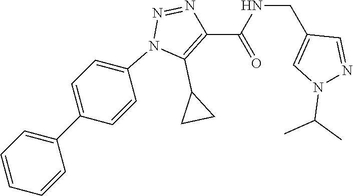 [SMILES: CC(C)n1cc(CNC(=O)c2nnn(-c3ccc(-c4ccccc4)cc3)c2C2CC2)cn1] |
| 14-33 | 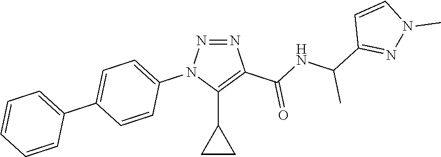 [SMILES: CC(NC(=O)c1nnn(-c2ccc(-c3ccccc3)cc2)c1C1CC1)c1ccn(C)n1] |
| 14-34 | 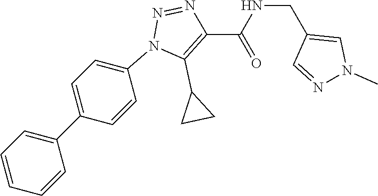 [SMILES: Cn1cc(CNC(=O)c2nnn(-c3ccc(-c4ccccc4)cc3)c2C2CC2)cn1] |
| 14-36 | 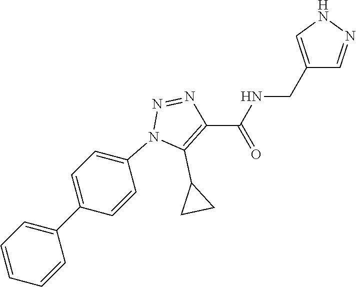 [SMILES: O=C(NCc1cn[nH]c1)c1nnn(-c2ccc(-c3ccccc3)cc2)c1C1CC1] |
| 14-37 | 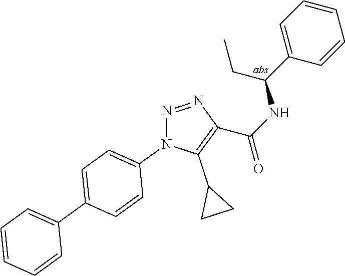 [SMILES: CC[C@H](NC(=O)c1nnn(-c2ccc(-c3ccccc3)cc2)c1C1CC1)c1ccccc1] |
| 14-38 | 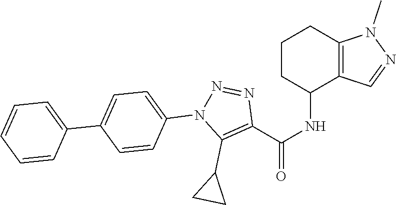 [SMILES: Cn1ncc2c1CCCC2NC(=O)c1nnn(-c2ccc(-c3ccccc3)cc2)c1C1CC1] |
| 14-39 | 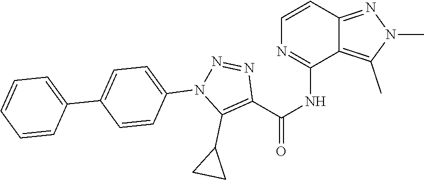 [SMILES: Cc1c2c(NC(=O)c3nnn(-c4ccc(-c5ccccc5)cc4)c3C3CC3)nccc2nn1C] |
| 14-40 | 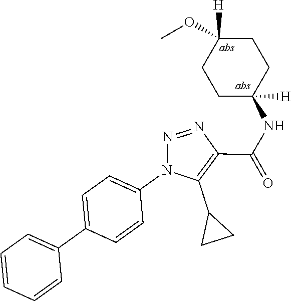 [SMILES: CO[C@H]1CC[C@H](NC(=O)c2nnn(-c3ccc(-c4ccccc4)cc3)c2C2CC2)CC1] |
| 14-41 | 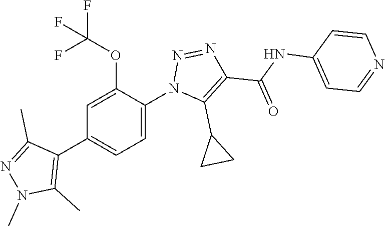 [SMILES: Cc1nn(C)c(C)c1-c1ccc(-n2nnc(C(=O)Nc3ccncc3)c2C2CC2)c(OC(F)(F)F)c1] |
| 14-42 | 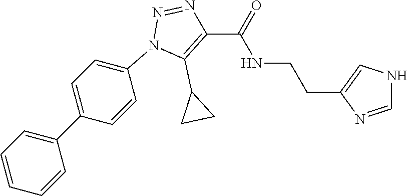 [SMILES: O=C(NCCc1c[nH]cn1)c1nnn(-c2ccc(-c3ccccc3)cc2)c1C1CC1] |
| 14-43 | 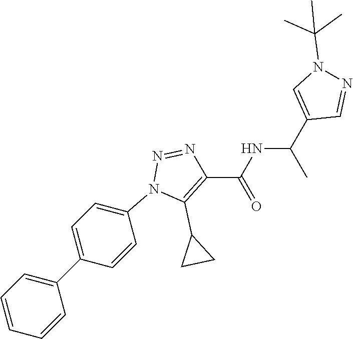 [SMILES: CC(NC(=O)c1nnn(-c2ccc(-c3ccccc3)cc2)c1C1CC1)c1cnn(C(C)(C)C)c1] |
| 14-44 | 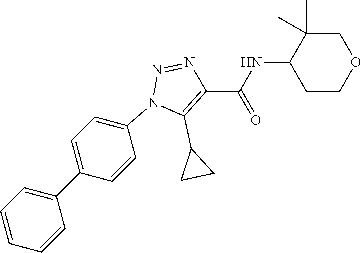 [SMILES: CC1(C)COCCC1NC(=O)c1nnn(-c2ccc(-c3ccccc3)cc2)c1C1CC1] |
| 14-45 | 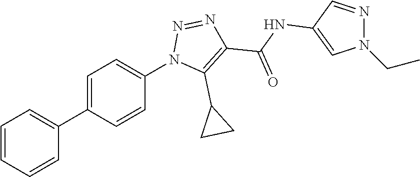 [SMILES: CCn1cc(NC(=O)c2nnn(-c3ccc(-c4ccccc4)cc3)c2C2CC2)cn1] |
| 14-46 | 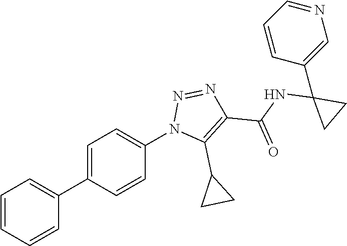 [SMILES: O=C(NC1(c2cccnc2)CC1)c1nnn(-c2ccc(-c3ccccc3)cc2)c1C1CC1] |
| 14-47 | 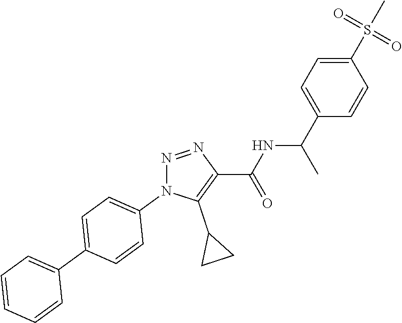 [SMILES: CC(NC(=O)c1nnn(-c2ccc(-c3ccccc3)cc2)c1C1CC1)c1ccc(S(C)(=O)=O)cc1] |
| 14-48 | 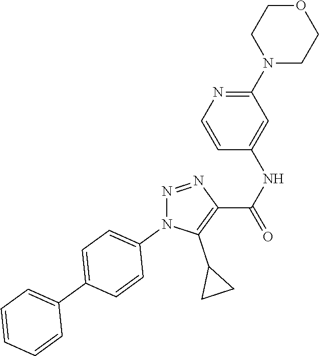 [SMILES: O=C(Nc1ccnc(N2CCOCC2)c1)c1nnn(-c2ccc(-c3ccccc3)cc2)c1C1CC1] |
| 14-49 | 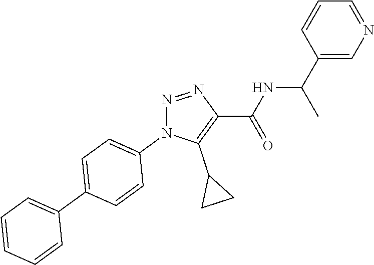 [SMILES: CC(NC(=O)c1nnn(-c2ccc(-c3ccccc3)cc2)c1C1CC1)c1cccnc1] |
| 14-50 | 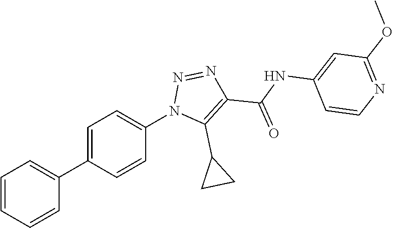 [SMILES: COc1cc(NC(=O)c2nnn(-c3ccc(-c4ccccc4)cc3)c2C2CC2)ccn1] |
| 14-51 | 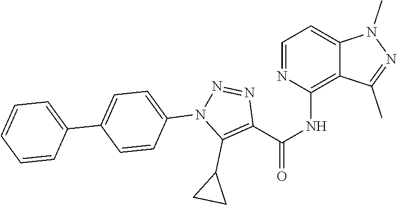 [SMILES: Cc1nn(C)c2ccnc(NC(=O)c3nnn(-c4ccc(-c5ccccc5)cc4)c3C3CC3)c12] |
| 14-52 | 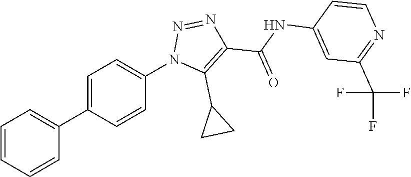 [SMILES: O=C(Nc1ccnc(C(F)(F)F)c1)c1nnn(-c2ccc(-c3ccccc3)cc2)c1C1CC1] |
| 14-53 | 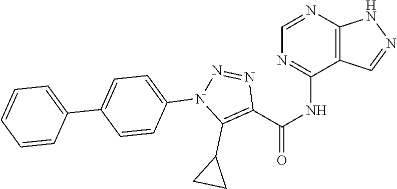 [SMILES: O=C(Nc1ncnc2[nH]ncc12)c1nnn(-c2ccc(-c3ccccc3)cc2)c1C1CC1] |
| 14-54 | 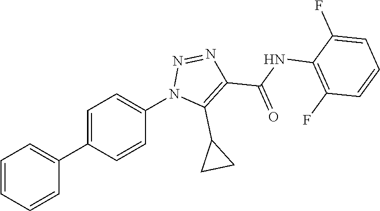 [SMILES: O=C(Nc1c(F)cccc1F)c1nnn(-c2ccc(-c3ccccc3)cc2)c1C1CC1] |
| 14-55 | 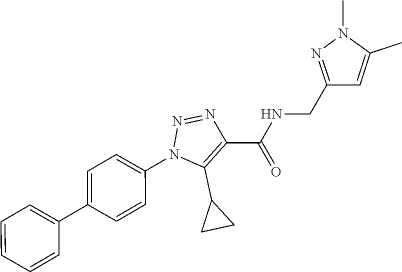 [SMILES: Cc1cc(CNC(=O)c2nnn(-c3ccc(-c4ccccc4)cc3)c2C2CC2)nn1C] |
| 14-56 | 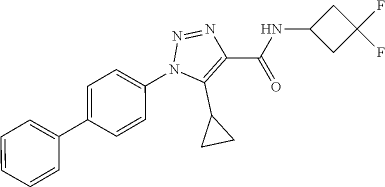 [SMILES: O=C(NC1CC(F)(F)C1)c1nnn(-c2ccc(-c3ccccc3)cc2)c1C1CC1] |
| 14-57 | 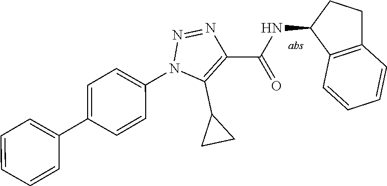 [SMILES: O=C(N[C@H]1CCc2ccccc21)c1nnn(-c2ccc(-c3ccccc3)cc2)c1C1CC1] |
| 14-58 | 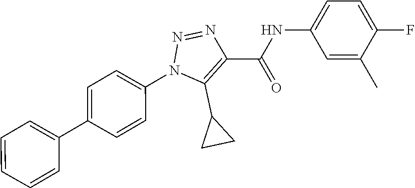 [SMILES: Cc1cc(NC(=O)c2nnn(-c3ccc(-c4ccccc4)cc3)c2C2CC2)ccc1F] |
| 14-59 | 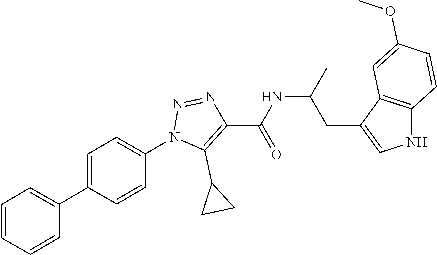 [SMILES: COc1ccc2[nH]cc(CC(C)NC(=O)c3nnn(-c4ccc(-c5ccccc5)cc4)c3C3CC3)c2c1] |
| 14-60 | 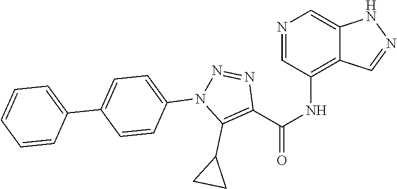 [SMILES: O=C(Nc1cncc2[nH]ncc12)c1nnn(-c2ccc(-c3ccccc3)cc2)c1C1CC1] |
| 14-61 | 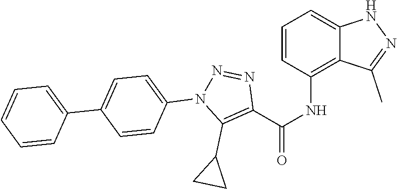 [SMILES: Cc1n[nH]c2cccc(NC(=O)c3nnn(-c4ccc(-c5ccccc5)cc4)c3C3CC3)c12] |
| 14-62 | 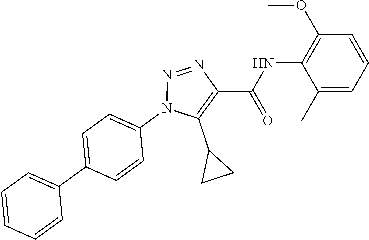 [SMILES: COc1cccc(C)c1NC(=O)c1nnn(-c2ccc(-c3ccccc3)cc2)c1C1CC1] |
| 14-63 | 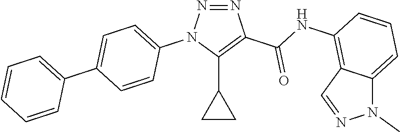 [SMILES: Cn1ncc2c(NC(=O)c3nnn(-c4ccc(-c5ccccc5)cc4)c3C3CC3)cccc21] |
| 14-64 | 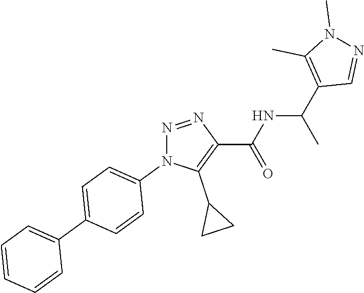 [SMILES: Cc1c(C(C)NC(=O)c2nnn(-c3ccc(-c4ccccc4)cc3)c2C2CC2)cnn1C] |
| 14-65 | 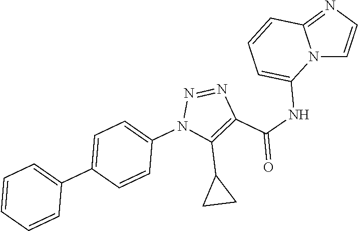 [SMILES: O=C(Nc1cccc2nccn12)c1nnn(-c2ccc(-c3ccccc3)cc2)c1C1CC1] |
| 14-66 | 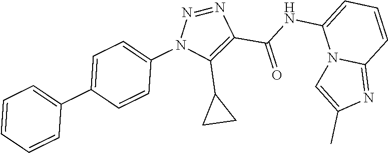 [SMILES: Cc1cn2c(NC(=O)c3nnn(-c4ccc(-c5ccccc5)cc4)c3C3CC3)cccc2n1] |
| 14-67 | 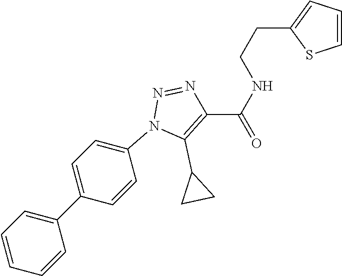 [SMILES: O=C(NCCc1cccs1)c1nnn(-c2ccc(-c3ccccc3)cc2)c1C1CC1] |
| 14-68 | 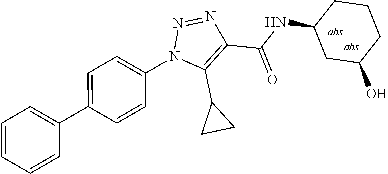 [SMILES: O=C(N[C@H]1CCC[C@@H](O)C1)c1nnn(-c2ccc(-c3ccccc3)cc2)c1C1CC1] |
| 14-69 | 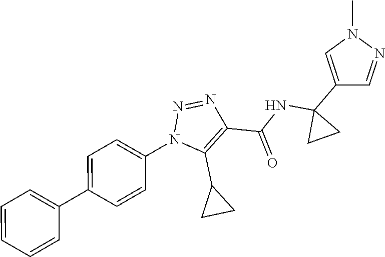 [SMILES: Cn1cc(C2(NC(=O)c3nnn(-c4ccc(-c5ccccc5)cc4)c3C3CC3)CC2)cn1] |
| 14-70 | 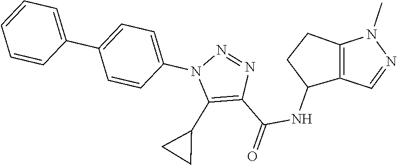 [SMILES: Cn1ncc2c1CCC2NC(=O)c1nnn(-c2ccc(-c3ccccc3)cc2)c1C1CC1] |
| 14-71 | 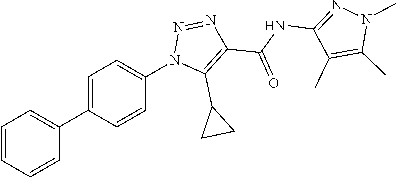 [SMILES: Cc1c(NC(=O)c2nnn(-c3ccc(-c4ccccc4)cc3)c2C2CC2)nn(C)c1C] |
| 14-72 | 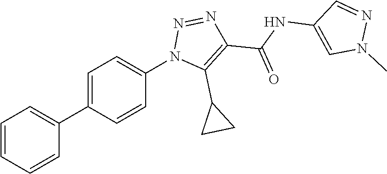 [SMILES: Cn1cc(NC(=O)c2nnn(-c3ccc(-c4ccccc4)cc3)c2C2CC2)cn1] |
| 14-73 | 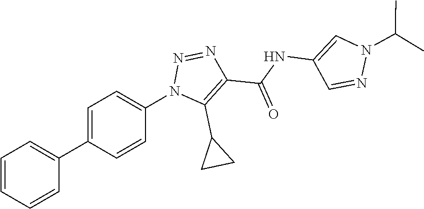 [SMILES: CC(C)n1cc(NC(=O)c2nnn(-c3ccc(-c4ccccc4)cc3)c2C2CC2)cn1] |
| 14-74 | 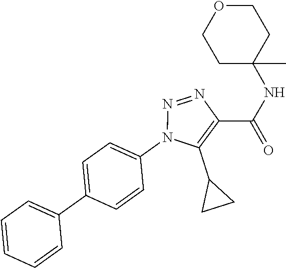 [SMILES: CC1(NC(=O)c2nnn(-c3ccc(-c4ccccc4)cc3)c2C2CC2)CCOCC1] |
| 14-75 | 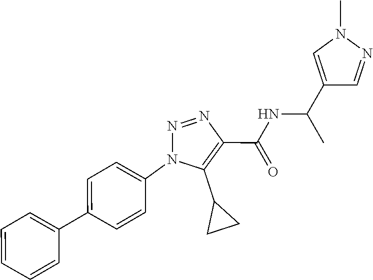 [SMILES: CC(NC(=O)c1nnn(-c2ccc(-c3ccccc3)cc2)c1C1CC1)c1cnn(C)c1] |
| 14-76 | 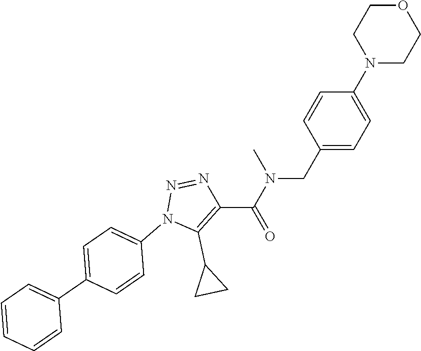 [SMILES: CN(Cc1ccc(N2CCOCC2)cc1)C(=O)c1nnn(-c2ccc(-c3ccccc3)cc2)c1C1CC1] |
| 14-77 | 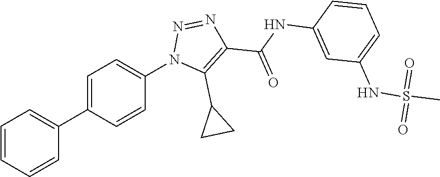 [SMILES: CS(=O)(=O)Nc1cccc(NC(=O)c2nnn(-c3ccc(-c4ccccc4)cc3)c2C2CC2)c1] |
| 14-78 | 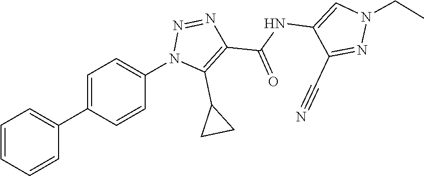 [SMILES: CCn1cc(NC(=O)c2nnn(-c3ccc(-c4ccccc4)cc3)c2C2CC2)c(C#N)n1] |
| 14-79 | 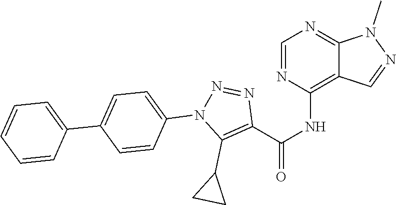 [SMILES: Cn1ncc2c(NC(=O)c3nnn(-c4ccc(-c5ccccc5)cc4)c3C3CC3)ncnc21] |
| 14-80 | 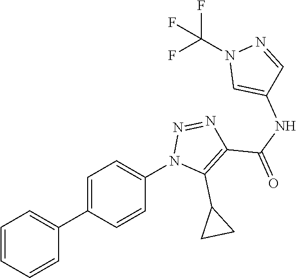 [SMILES: O=C(Nc1cnn(C(F)(F)F)c1)c1nnn(-c2ccc(-c3ccccc3)cc2)c1C1CC1] |
| 14-81 | 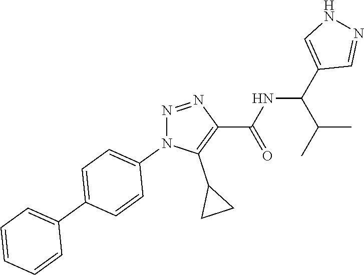 [SMILES: CC(C)C(NC(=O)c1nnn(-c2ccc(-c3ccccc3)cc2)c1C1CC1)c1cn[nH]c1] |
| 14-82 | 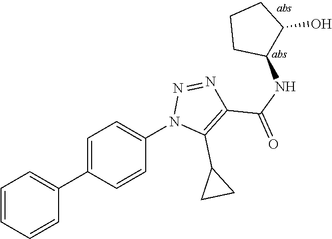 [SMILES: O=C(N[C@H]1CCC[C@@H]1O)c1nnn(-c2ccc(-c3ccccc3)cc2)c1C1CC1] |
| 14-83 | 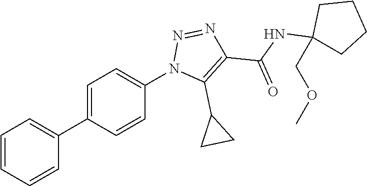 [SMILES: COCC1(NC(=O)c2nnn(-c3ccc(-c4ccccc4)cc3)c2C2CC2)CCCC1] |
| 14-84 | 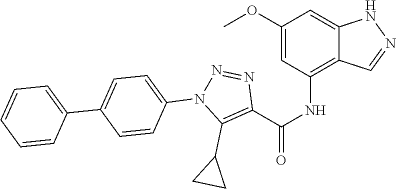 [SMILES: COc1cc(NC(=O)c2nnn(-c3ccc(-c4ccccc4)cc3)c2C2CC2)c2cn[nH]c2c1] |
| 14-85 | 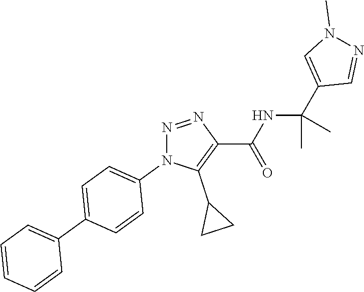 [SMILES: Cn1cc(C(C)(C)NC(=O)c2nnn(-c3ccc(-c4ccccc4)cc3)c2C2CC2)cn1] |
| 14-86 | 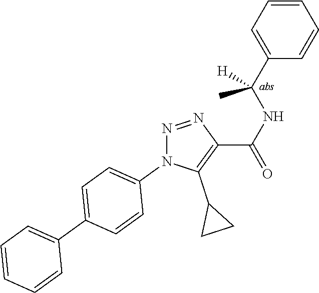 [SMILES: C[C@H](NC(=O)c1nnn(-c2ccc(-c3ccccc3)cc2)c1C1CC1)c1ccccc1] |
| 14-87 | 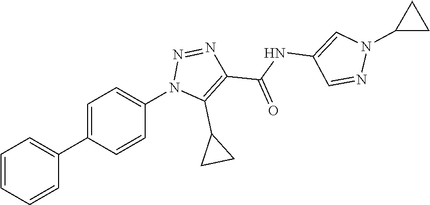 [SMILES: O=C(Nc1cnn(C2CC2)c1)c1nnn(-c2ccc(-c3ccccc3)cc2)c1C1CC1] |
| 14-88 | 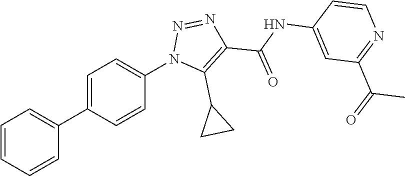 [SMILES: CC(=O)c1cc(NC(=O)c2nnn(-c3ccc(-c4ccccc4)cc3)c2C2CC2)ccn1] |
| 15-1 | 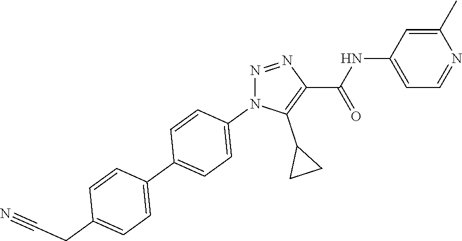 [SMILES: Cc1cc(NC(=O)c2nnn(-c3ccc(-c4ccc(CC#N)cc4)cc3)c2C2CC2)ccn1] |
| 15-2 | 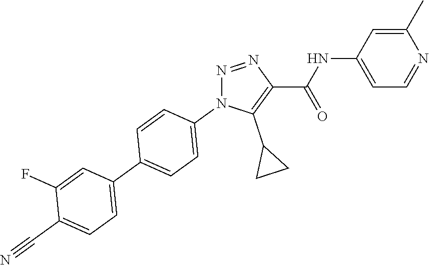 [SMILES: Cc1cc(NC(=O)c2nnn(-c3ccc(-c4ccc(C#N)c(F)c4)cc3)c2C2CC2)ccn1] |
| 15-3 | 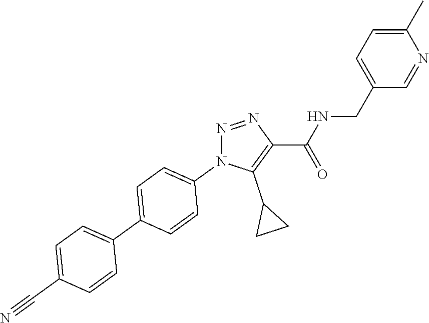 [SMILES: Cc1ccc(CNC(=O)c2nnn(-c3ccc(-c4ccc(C#N)cc4)cc3)c2C2CC2)cn1] |
| 15-4 | 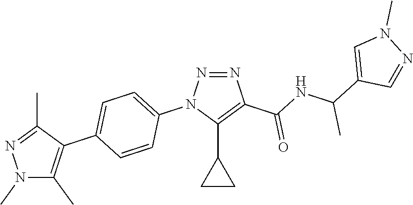 [SMILES: Cc1nn(C)c(C)c1-c1ccc(-n2nnc(C(=O)NC(C)c3cnn(C)c3)c2C2CC2)cc1] |
| 15-5 | 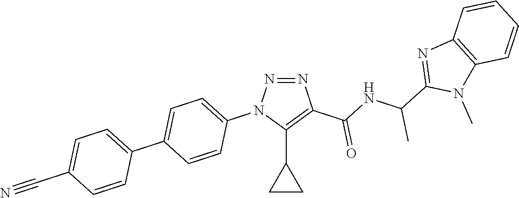 [SMILES: CC(NC(=O)c1nnn(-c2ccc(-c3ccc(C#N)cc3)cc2)c1C1CC1)c1nc2ccccc2n1C] |
| 15-6 | 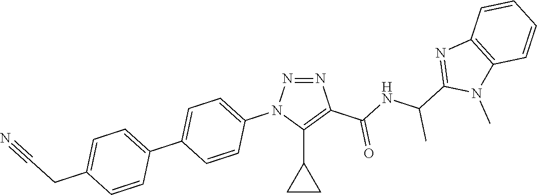 [SMILES: CC(NC(=O)c1nnn(-c2ccc(-c3ccc(CC#N)cc3)cc2)c1C1CC1)c1nc2ccccc2n1C] |
| 15-7 | 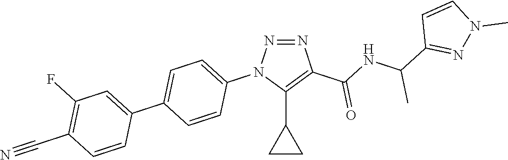 [SMILES: CC(NC(=O)c1nnn(-c2ccc(-c3ccc(C#N)c(F)c3)cc2)c1C1CC1)c1ccn(C)n1] |
| 15-8 | 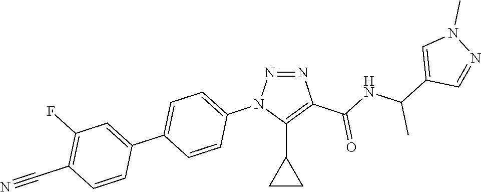 [SMILES: CC(NC(=O)c1nnn(-c2ccc(-c3ccc(C#N)c(F)c3)cc2)c1C1CC1)c1cnn(C)c1] |
| 15-9 | 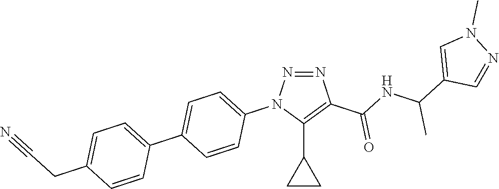 [SMILES: CC(NC(=O)c1nnn(-c2ccc(-c3ccc(CC#N)cc3)cc2)c1C1CC1)c1cnn(C)c1] |
| 15-10 | 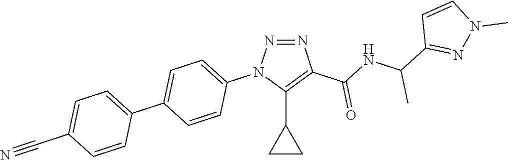 [SMILES: CC(NC(=O)c1nnn(-c2ccc(-c3ccc(C#N)cc3)cc2)c1C1CC1)c1ccn(C)n1] |
| 15-11 | 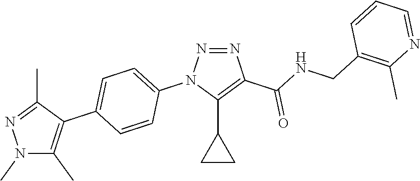 [SMILES: Cc1ncccc1CNC(=O)c1nnn(-c2ccc(-c3c(C)nn(C)c3C)cc2)c1C1CC1] |
| 15-12 | 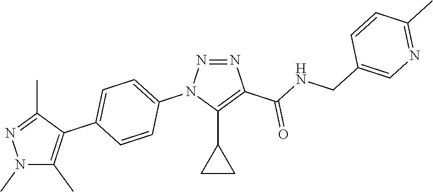 |
| 15-13 | 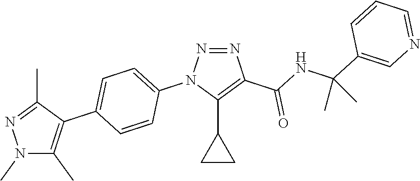 [SMILES: Cc1nn(C)c(C)c1-c1ccc(-n2nnc(C(=O)NC(C)(C)c3cccnc3)c2C2CC2)cc1] |
| 15-14 | 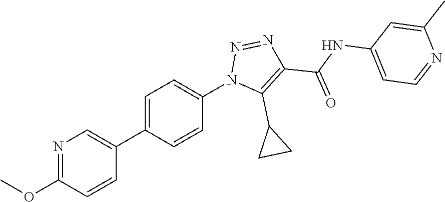 |
| 15-15 | 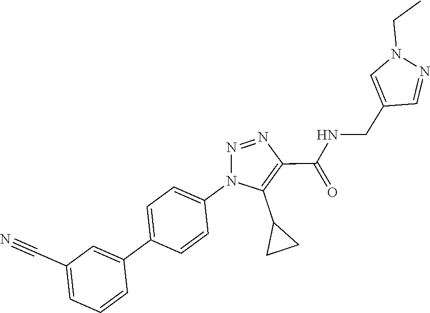 [SMILES: CCn1cc(CNC(=O)c2nnn(-c3ccc(-c4cccc(C#N)c4)cc3)c2C2CC2)cn1] |
| 15-16 | 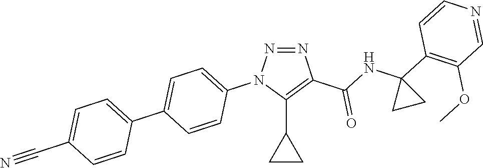 [SMILES: COc1cnccc1C1(NC(=O)c2nnn(-c3ccc(-c4ccc(C#N)cc4)cc3)c2C2CC2)CC1] |
| 15-17 | 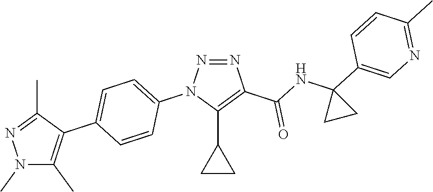 |
| 15-18 | 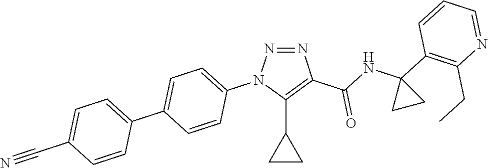 [SMILES: CCc1ncccc1C1(NC(=O)c2nnn(-c3ccc(-c4ccc(C#N)cc4)cc3)c2C2CC2)CC1] |
| 15-19 | 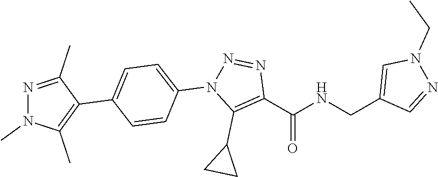 [SMILES: CCn1cc(CNC(=O)c2nnn(-c3ccc(-c4c(C)nn(C)c4C)cc3)c2C2CC2)cn1] |
| 15-20 | 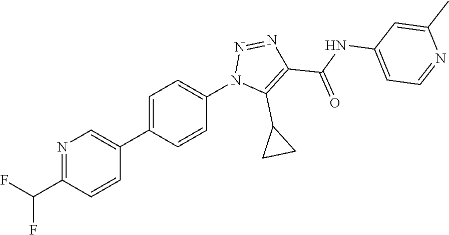 [SMILES: Cc1cc(NC(=O)c2nnn(-c3ccc(-c4ccc(C(F)F)nc4)cc3)c2C2CC2)ccn1] |
| 15-21 | 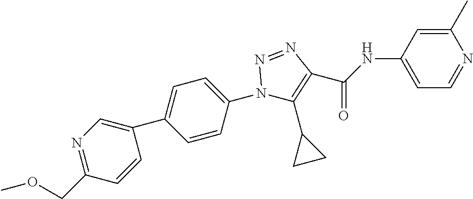 |
| 15-22 | 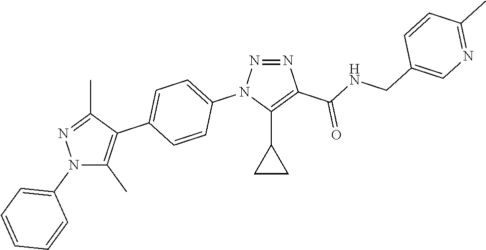 [SMILES: Cc1ccc(CNC(=O)c2nnn(-c3ccc(-c4c(C)nn(-c5ccccc5)c4C)cc3)c2C2CC2)cn1] |
| 15-23 | 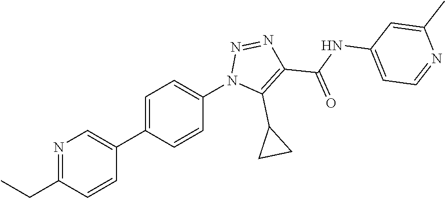 [SMILES: CCc1ccc(-c2ccc(-n3nnc(C(=O)Nc4ccnc(C)c4)c3C3CC3)cc2)cn1] |
| 15-24 | 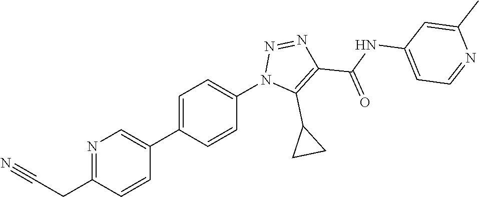 [SMILES: Cc1cc(NC(=O)c2nnn(-c3ccc(-c4ccc(CC#N)nc4)cc3)c2C2CC2)ccn1] |
| 16-1 | 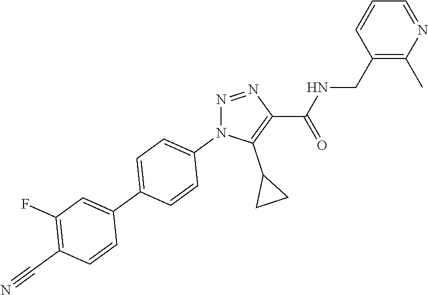 [SMILES: Cc1ncccc1CNC(=O)c1nnn(-c2ccc(-c3ccc(C#N)c(F)c3)cc2)c1C1CC1] |
| 16-2 | 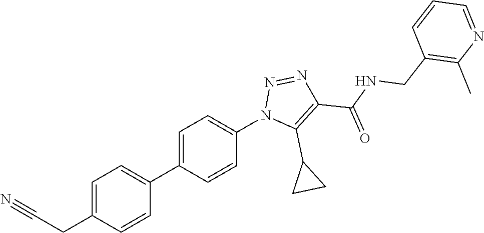 [SMILES: Cc1ncccc1CNC(=O)c1nnn(-c2ccc(-c3ccc(CC#N)cc3)cc2)c1C1CC1] |
| 16-3 | 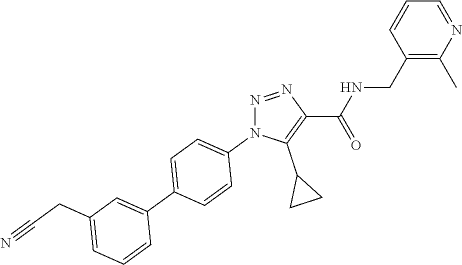 [SMILES: Cc1ncccc1CNC(=O)c1nnn(-c2ccc(-c3cccc(CC#N)c3)cc2)c1C1CC1] |
| 16-4 | 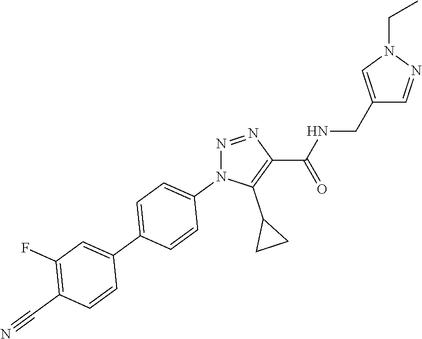 [SMILES: CCn1cc(CNC(=O)c2nnn(-c3ccc(-c4ccc(C#N)c(F)c4)cc3)c2C2CC2)cn1] |
| 16-5 | 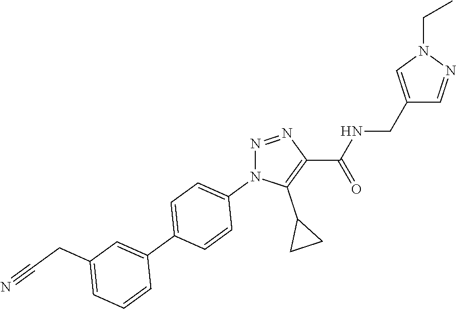 [SMILES: CCn1cc(CNC(=O)c2nnn(-c3ccc(-c4cccc(CC#N)c4)cc3)c2C2CC2)cn1] |
| 17-1 | 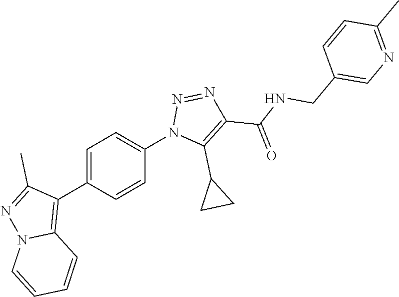 [SMILES: Cc1ccc(CNC(=O)c2nnn(-c3ccc(-c4c(C)nn5ccccc45)cc3)c2C2CC2)cn1] |
| 17-2 | 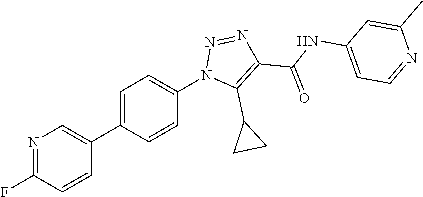 [SMILES: Cc1cc(NC(=O)c2nnn(-c3ccc(-c4ccc(F)nc4)cc3)c2C2CC2)ccn1] |
| 17-3 | 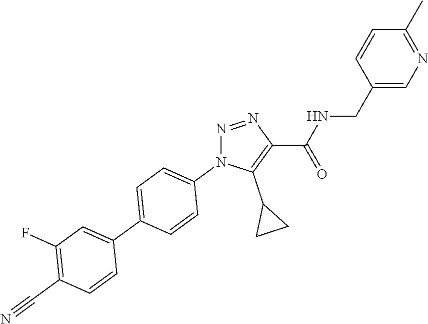 [SMILES: Cc1ccc(CNC(=O)c2nnn(-c3ccc(-c4ccc(C#N)c(F)c4)cc3)c2C2CC2)cn1] |
| 17-4 | 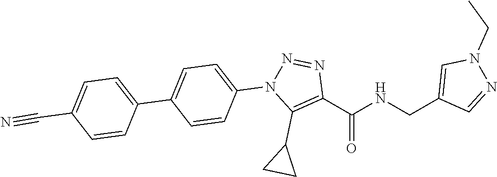 [SMILES: CCn1cc(CNC(=O)c2nnn(-c3ccc(-c4ccc(C#N)cc4)cc3)c2C2CC2)cn1] |
| 17-5 | 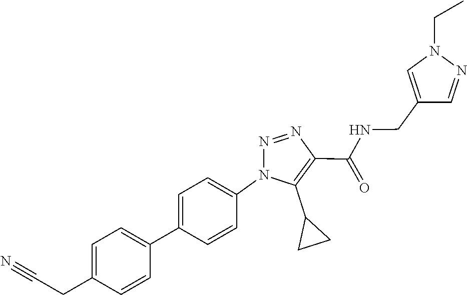 [SMILES: CCn1cc(CNC(=O)c2nnn(-c3ccc(-c4ccc(CC#N)cc4)cc3)c2C2CC2)cn1] |
| 17-6 | 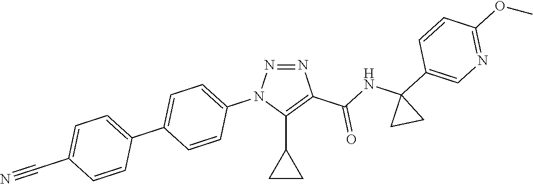 [SMILES: COc1ccc(C2(NC(=O)c3nnn(-c4ccc(-c5ccc(C#N)cc5)cc4)c3C3CC3)CC2)cn1] |
| 17-7 | 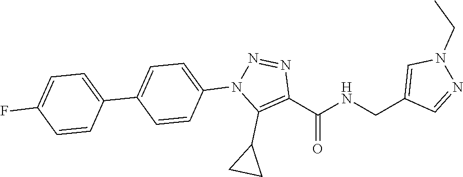 [SMILES: CCn1cc(CNC(=O)c2nnn(-c3ccc(-c4ccc(F)cc4)cc3)c2C2CC2)cn1] |
| 17-8 | 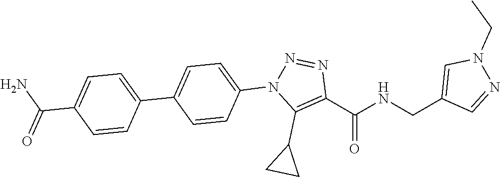 [SMILES: CCn1cc(CNC(=O)c2nnn(-c3ccc(-c4ccc(C(N)=O)cc4)cc3)c2C2CC2)cn1] |
| 17-9 | 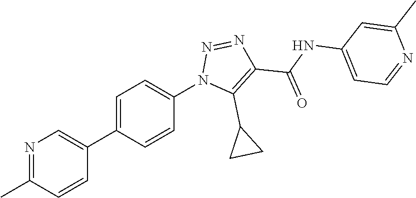 |
| 17-10 | 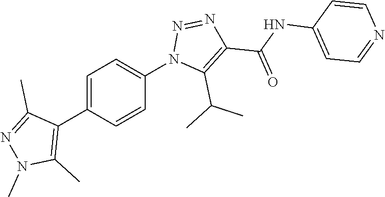 [SMILES: Cc1nn(C)c(C)c1-c1ccc(-n2nnc(C(=O)Nc3ccncc3)c2C(C)C)cc1] |
| 17-11 | 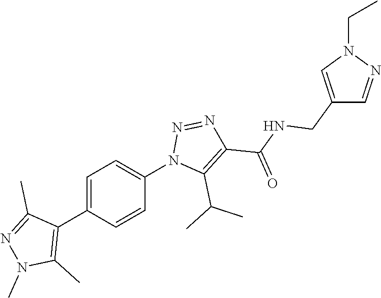 [SMILES: CCn1cc(CNC(=O)c2nnn(-c3ccc(-c4c(C)nn(C)c4C)cc3)c2C(C)C)cn1] |
| 17-12 | 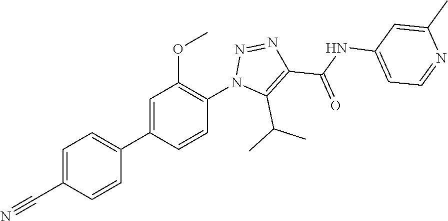 [SMILES: COc1cc(-c2ccc(C#N)cc2)ccc1-n1nnc(C(=O)Nc2ccnc(C)c2)c1C(C)C] |
| 17-13 | 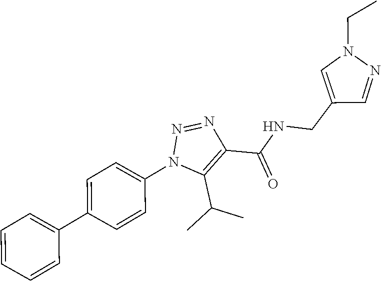 [SMILES: CCn1cc(CNC(=O)c2nnn(-c3ccc(-c4ccccc4)cc3)c2C(C)C)cn1] |
| 17-14 | 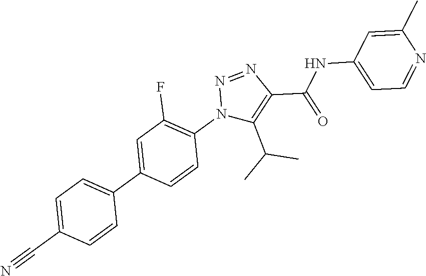 [SMILES: Cc1cc(NC(=O)c2nnn(-c3ccc(-c4ccc(C#N)cc4)cc3F)c2C(C)C)ccn1] |
| 17-15 | 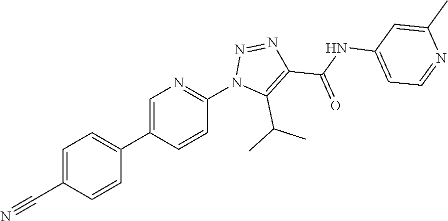 [SMILES: Cc1cc(NC(=O)c2nnn(-c3ccc(-c4ccc(C#N)cc4)cn3)c2C(C)C)ccn1] |
| 17-16 | 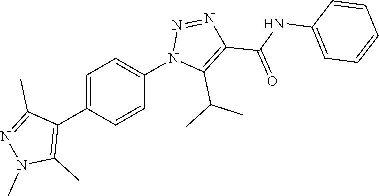 [SMILES: Cc1nn(C)c(C)c1-c1ccc(-n2nnc(C(=O)Nc3ccccc3)c2C(C)C)cc1] |
| 17-17 | 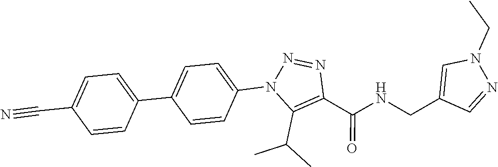 [SMILES: CCn1cc(CNC(=O)c2nnn(-c3ccc(-c4ccc(C#N)cc4)cc3)c2C(C)C)cn1] |
| 17-18 | 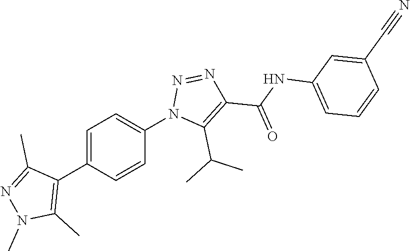 [SMILES: Cc1nn(C)c(C)c1-c1ccc(-n2nnc(C(=O)Nc3cccc(C#N)c3)c2C(C)C)cc1] |
| 17-19 | 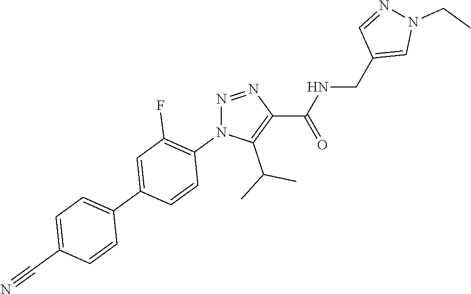 [SMILES: CCn1cc(CNC(=O)c2nnn(-c3ccc(-c4ccc(C#N)cc4)cc3F)c2C(C)C)cn1] |
| 17-20 | 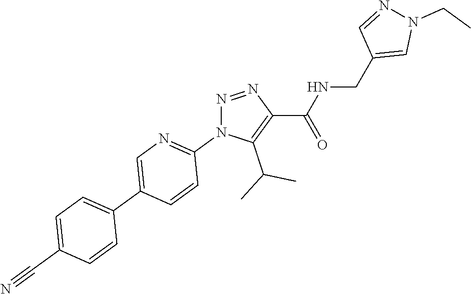 [SMILES: CCn1cc(CNC(=O)c2nnn(-c3ccc(-c4ccc(C#N)cc4)cn3)c2C(C)C)cn1] |
| 17-21 | 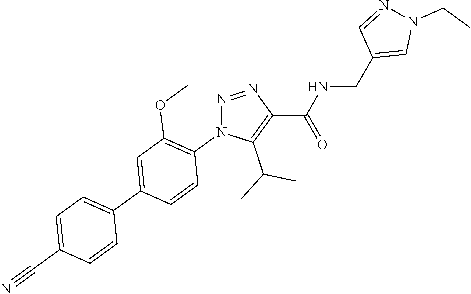 [SMILES: CCn1cc(CNC(=O)c2nnn(-c3ccc(-c4ccc(C#N)cc4)cc3OC)c2C(C)C)cn1] |
| 17-22 | 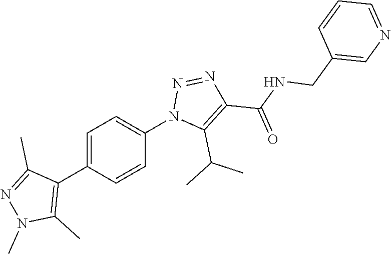 [SMILES: Cc1nn(C)c(C)c1-c1ccc(-n2nnc(C(=O)NCc3cccnc3)c2C(C)C)cc1] |
| 17-23 | 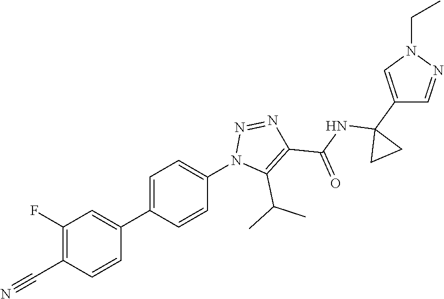 [SMILES: CCn1cc(C2(NC(=O)c3nnn(-c4ccc(-c5ccc(C#N)c(F)c5)cc4)c3C(C)C)CC2)cn1] |
| 17-24 | 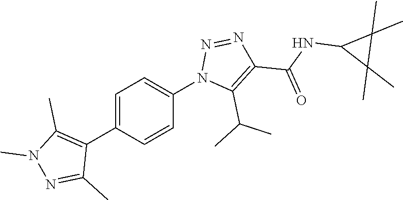 |
| 17-25 | 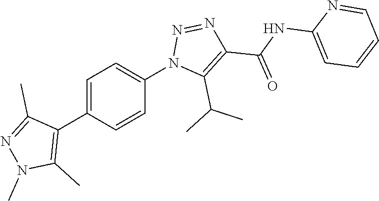 [SMILES: Cc1nn(C)c(C)c1-c1ccc(-n2nnc(C(=O)Nc3ccccn3)c2C(C)C)cc1] |
| 17-26 | 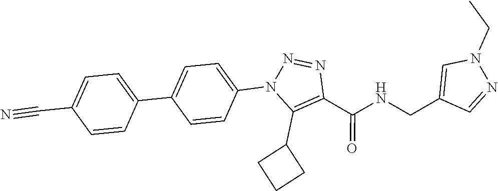 [SMILES: CCn1cc(CNC(=O)c2nnn(-c3ccc(-c4ccc(C#N)cc4)cc3)c2C2CCC2)cn1] |
| 17-27 | 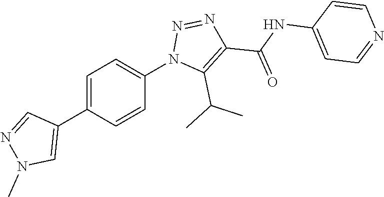 [SMILES: CC(C)c1c(C(=O)Nc2ccncc2)nnn1-c1ccc(-c2cnn(C)c2)cc1] |
| 17-28 | 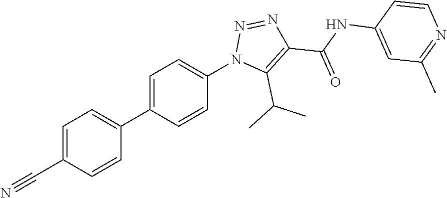 [SMILES: Cc1cc(NC(=O)c2nnn(-c3ccc(-c4ccc(C#N)cc4)cc3)c2C(C)C)ccn1] |
| 17-29 | 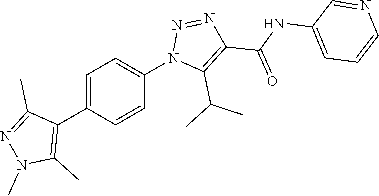 [SMILES: Cc1nn(C)c(C)c1-c1ccc(-n2nnc(C(=O)Nc3cccnc3)c2C(C)C)cc1] |
| 17-30 | 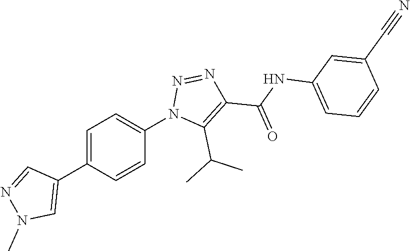 [SMILES: CC(C)c1c(C(=O)Nc2cccc(C#N)c2)nnn1-c1ccc(-c2cnn(C)c2)cc1] |
| 17-31 | 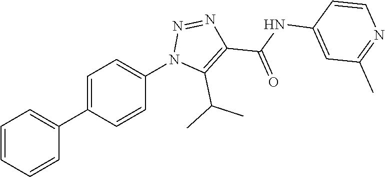 [SMILES: Cc1cc(NC(=O)c2nnn(-c3ccc(-c4ccccc4)cc3)c2C(C)C)ccn1] |
| 17-32 | 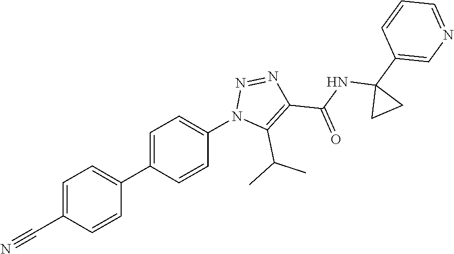 [SMILES: CC(C)c1c(C(=O)NC2(c3cccnc3)CC2)nnn1-c1ccc(-c2ccc(C#N)cc2)cc1] |
| 17-33 | 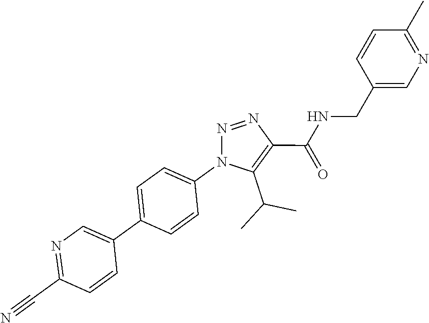 [SMILES: Cc1ccc(CNC(=O)c2nnn(-c3ccc(-c4ccc(C#N)nc4)cc3)c2C(C)C)cn1] |
| 17-34 | 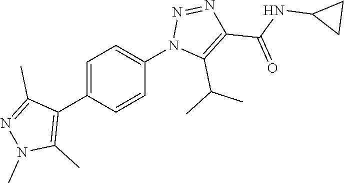 [SMILES: Cc1nn(C)c(C)c1-c1ccc(-n2nnc(C(=O)NC3CC3)c2C(C)C)cc1] |
| 18-1 | 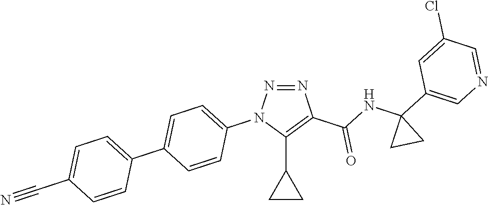 [SMILES: N#Cc1ccc(-c2ccc(-n3nnc(C(=O)NC4(c5cncc(Cl)c5)CC4)c3C3CC3)cc2)cc1] |
| 18-2 | 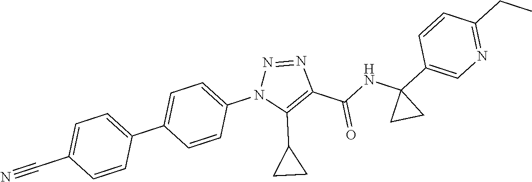 [SMILES: CCc1ccc(C2(NC(=O)c3nnn(-c4ccc(-c5ccc(C#N)cc5)cc4)c3C3CC3)CC2)cn1] |
| 18-3 | 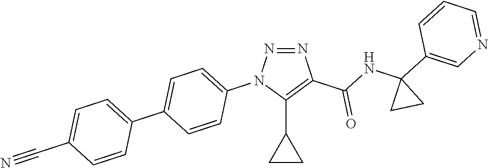 [SMILES: N#Cc1ccc(-c2ccc(-n3nnc(C(=O)NC4(c5cccnc5)CC4)c3C3CC3)cc2)cc1] |
| 18-4 | 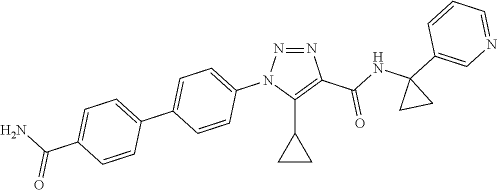 [SMILES: NC(=O)c1ccc(-c2ccc(-n3nnc(C(=O)NC4(c5cccnc5)CC4)c3C3CC3)cc2)cc1] |
| 18-5 | 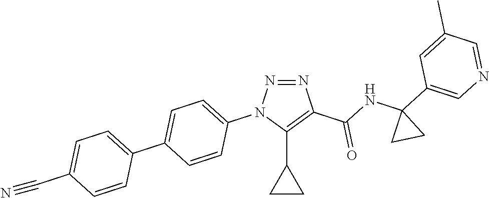 [SMILES: Cc1cncc(C2(NC(=O)c3nnn(-c4ccc(-c5ccc(C#N)cc5)cc4)c3C3CC3)CC2)c1] |
| 18-6 | 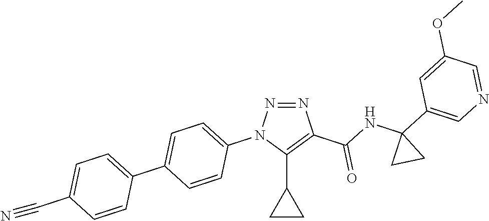 [SMILES: COc1cncc(C2(NC(=O)c3nnn(-c4ccc(-c5ccc(C#N)cc5)cc4)c3C3CC3)CC2)c1] |
| 18-7 | 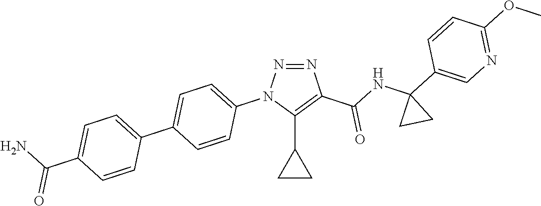 [SMILES: COc1ccc(C2(NC(=O)c3nnn(-c4ccc(-c5ccc(C(N)=O)cc5)cc4)c3C3CC3)CC2)cn1] |
| 18-8 | 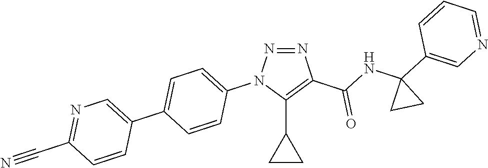 [SMILES: N#Cc1ccc(-c2ccc(-n3nnc(C(=O)NC4(c5cccnc5)CC4)c3C3CC3)cc2)cn1] |
| 18-9 | 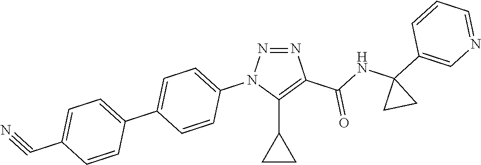 [SMILES: N#Cc1ccc(-c2ccc(-n3nnc(C(=O)NC4(c5cccnc5)CC4)c3C3CC3)cc2)cc1] |
| 18-10 | 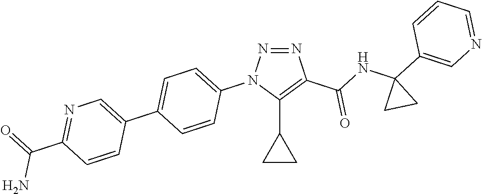 [SMILES: NC(=O)c1ccc(-c2ccc(-n3nnc(C(=O)NC4(c5cccnc5)CC4)c3C3CC3)cc2)cn1] |
| 18-11 | 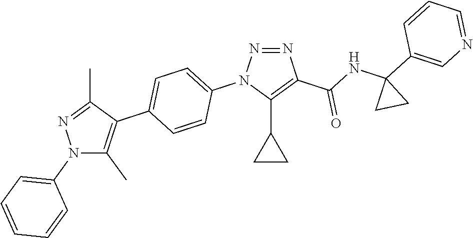 [SMILES: Cc1nn(-c2ccccc2)c(C)c1-c1ccc(-n2nnc(C(=O)NC3(c4cccnc4)CC3)c2C2CC2)cc1] |
| 18-12 | 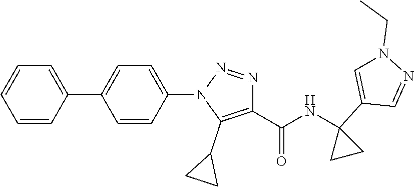 [SMILES: CCn1cc(C2(NC(=O)c3nnn(-c4ccc(-c5ccccc5)cc4)c3C3CC3)CC2)cn1] |
| 18-13 | 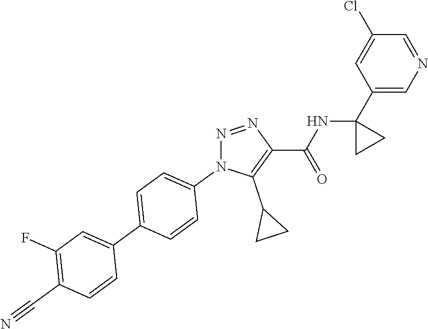 [SMILES: N#Cc1ccc(-c2ccc(-n3nnc(C(=O)NC4(c5cncc(Cl)c5)CC4)c3C3CC3)cc2)cc1F] |
| 18-14 | 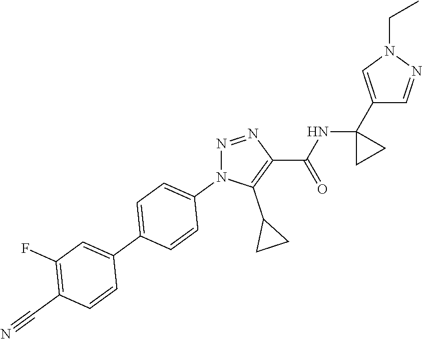 [SMILES: CCn1cc(C2(NC(=O)c3nnn(-c4ccc(-c5ccc(C#N)c(F)c5)cc4)c3C3CC3)CC2)cn1] |
| 18-15 | 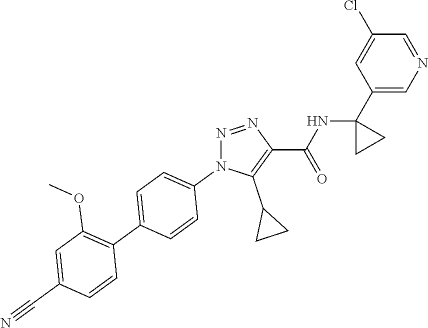 [SMILES: COc1cc(C#N)ccc1-c1ccc(-n2nnc(C(=O)NC3(c4cncc(Cl)c4)CC3)c2C2CC2)cc1] |
| 18-16 | 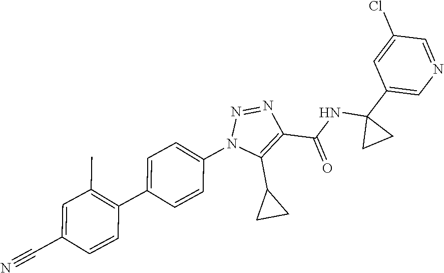 [SMILES: Cc1cc(C#N)ccc1-c1ccc(-n2nnc(C(=O)NC3(c4cncc(Cl)c4)CC3)c2C2CC2)cc1] |
| 18-17 | 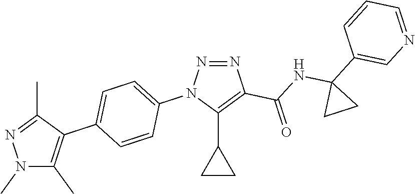 [SMILES: Cc1nn(C)c(C)c1-c1ccc(-n2nnc(C(=O)NC3(c4cccnc4)CC3)c2C2CC2)cc1] |
| 18-18 | 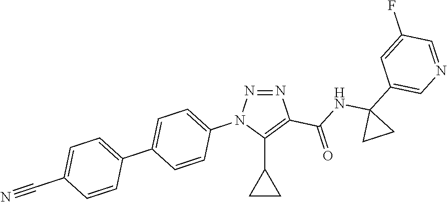 [SMILES: N#Cc1ccc(-c2ccc(-n3nnc(C(=O)NC4(c5cncc(F)c5)CC4)c3C3CC3)cc2)cc1] |
| 18-19 | 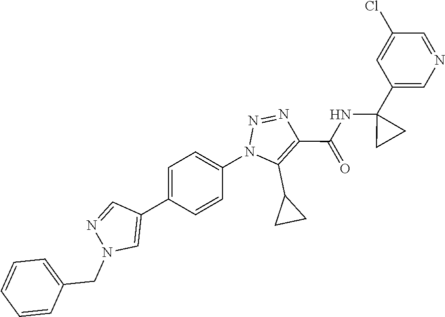 [SMILES: O=C(NC1(c2cncc(Cl)c2)CC1)c1nnn(-c2ccc(-c3cnn(Cc4ccccc4)c3)cc2)c1C1CC1] |
| 18-20 | 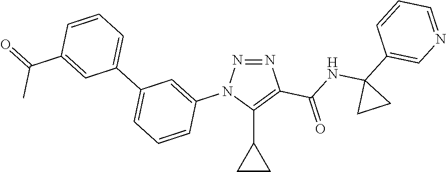 [SMILES: CC(=O)c1cccc(-c2cccc(-n3nnc(C(=O)NC4(c5cccnc5)CC4)c3C3CC3)c2)c1] |
| 18-21 | 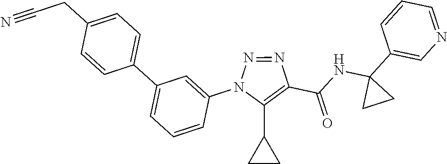 [SMILES: N#CCc1ccc(-c2cccc(-n3nnc(C(=O)NC4(c5cccnc5)CC4)c3C3CC3)c2)cc1] |
| 18-22 | 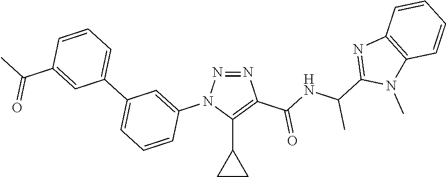 [SMILES: CC(=O)c1cccc(-c2cccc(-n3nnc(C(=O)NC(C)c4nc5ccccc5n4C)c3C3CC3)c2)c1] |
| 18-23 | 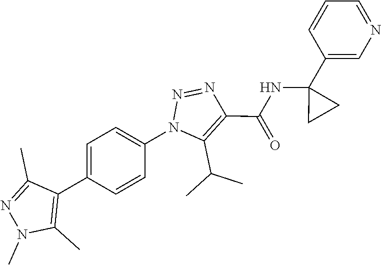 [SMILES: Cc1nn(C)c(C)c1-c1ccc(-n2nnc(C(=O)NC3(c4cccnc4)CC3)c2C(C)C)cc1] |
| 18-24 | 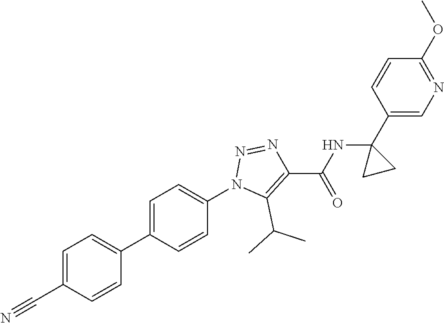 [SMILES: COc1ccc(C2(NC(=O)c3nnn(-c4ccc(-c5ccc(C#N)cc5)cc4)c3C(C)C)CC2)cn1] |
| 18-25 | 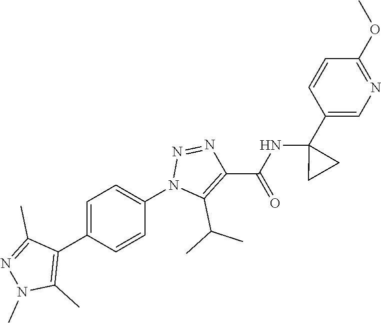 [SMILES: COc1ccc(C2(NC(=O)c3nnn(-c4ccc(-c5c(C)nn(C)c5C)cc4)c3C(C)C)CC2)cn1] |
| 18-26 | 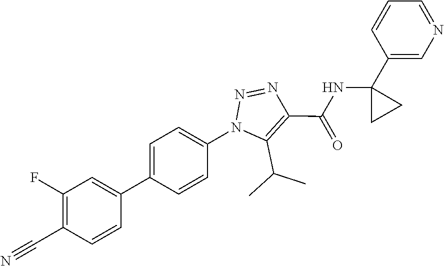 [SMILES: CC(C)c1c(C(=O)NC2(c3cccnc3)CC2)nnn1-c1ccc(-c2ccc(C#N)c(F)c2)cc1] |
| 18-27 | 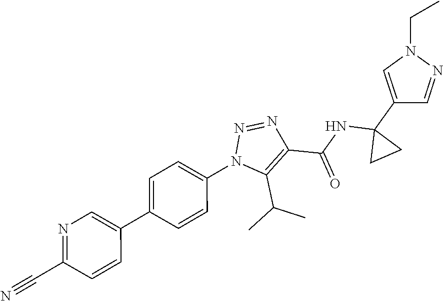 [SMILES: CCn1cc(C2(NC(=O)c3nnn(-c4ccc(-c5ccc(C#N)nc5)cc4)c3C(C)C)CC2)cn1] |
| 18-28 | 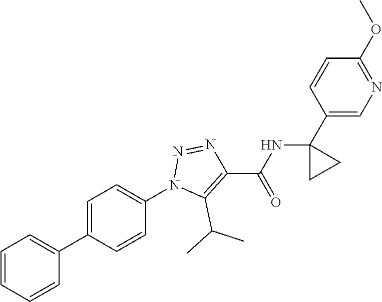 [SMILES: COc1ccc(C2(NC(=O)c3nnn(-c4ccc(-c5ccccc5)cc4)c3C(C)C)CC2)cn1] |
| 18-29 | 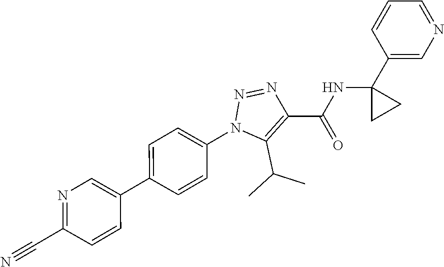 [SMILES: CC(C)c1c(C(=O)NC2(c3cccnc3)CC2)nnn1-c1ccc(-c2ccc(C#N)nc2)cc1] |
| 18-30 | 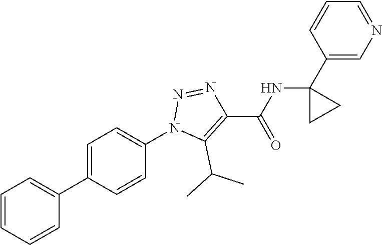 [SMILES: CC(C)c1c(C(=O)NC2(c3cccnc3)CC2)nnn1-c1ccc(-c2ccccc2)cc1] |
| 19-1 | 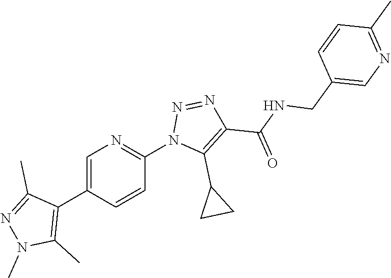 [SMILES: Cc1ccc(CNC(=O)c2nnn(-c3ccc(-c4c(C)nn(C)c4C)cn3)c2C2CC2)cn1] |
| 19-2 | 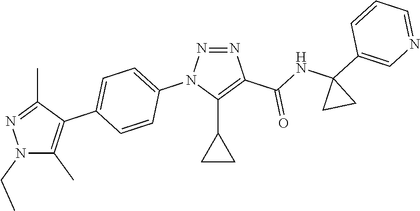 [SMILES: CCn1nc(C)c(-c2ccc(-n3nnc(C(=O)NC4(c5cccnc5)CC4)c3C3CC3)cc2)c1C] |
| 19-3 | 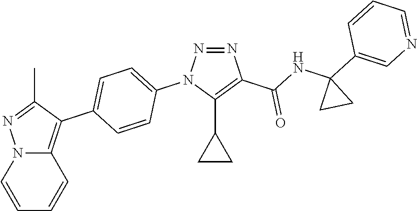 [SMILES: Cc1nn2ccccc2c1-c1ccc(-n2nnc(C(=O)NC3(c4cccnc4)CC3)c2C2CC2)cc1] |
| 19-4 | 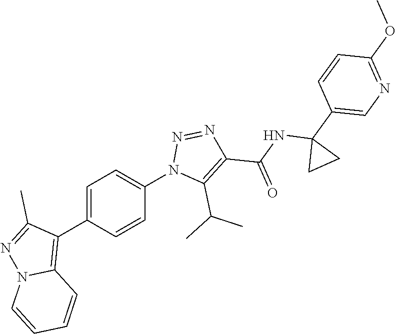 [SMILES: COc1ccc(C2(NC(=O)c3nnn(-c4ccc(-c5c(C)nn6ccccc56)cc4)c3C(C)C)CC2)cn1] |
| 20-1 | 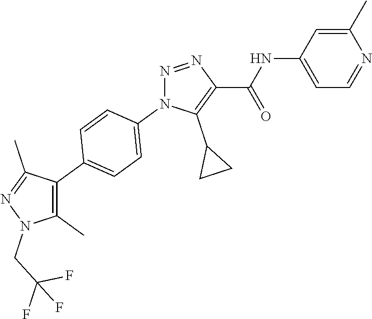 [SMILES: Cc1cc(NC(=O)c2nnn(-c3ccc(-c4c(C)nn(CC(F)(F)F)c4C)cc3)c2C2CC2)ccn1] |
| 21-1 | 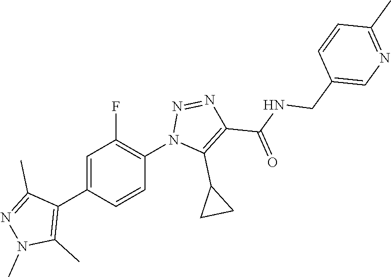 [SMILES: Cc1ccc(CNC(=O)c2nnn(-c3ccc(-c4c(C)nn(C)c4C)cc3F)c2C2CC2)cn1] |
| 21-2 | 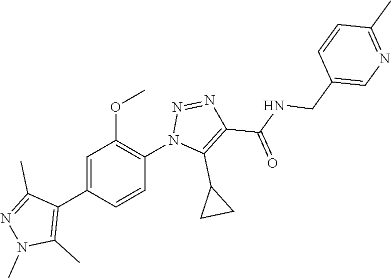 [SMILES: COc1cc(-c2c(C)nn(C)c2C)ccc1-n1nnc(C(=O)NCc2ccc(C)nc2)c1C1CC1] |
| 22-1 | 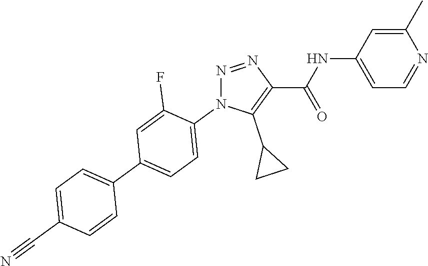 [SMILES: Cc1cc(NC(=O)c2nnn(-c3ccc(-c4ccc(C#N)cc4)cc3F)c2C2CC2)ccn1] |
| 22-2 | 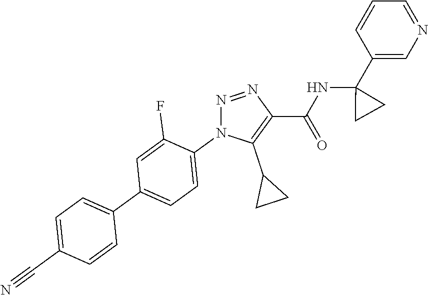 [SMILES: N#Cc1ccc(-c2ccc(-n3nnc(C(=O)NC4(c5cccnc5)CC4)c3C3CC3)c(F)c2)cc1] |
| 22-3 | 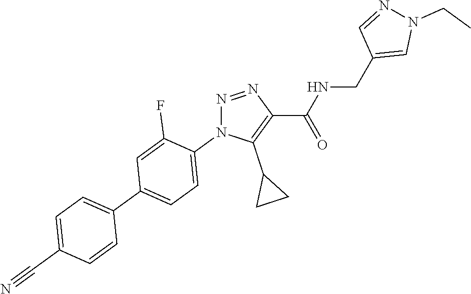 [SMILES: CCn1cc(CNC(=O)c2nnn(-c3ccc(-c4ccc(C#N)cc4)cc3F)c2C2CC2)cn1] |
| 22-4 | 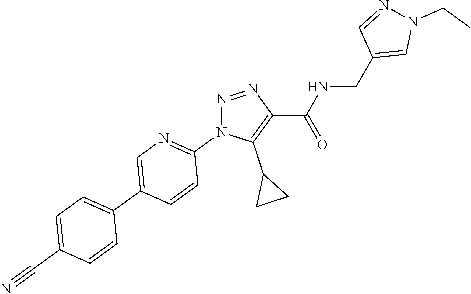 [SMILES: CCn1cc(CNC(=O)c2nnn(-c3ccc(-c4ccc(C#N)cc4)cn3)c2C2CC2)cn1] |
| 23-1 | 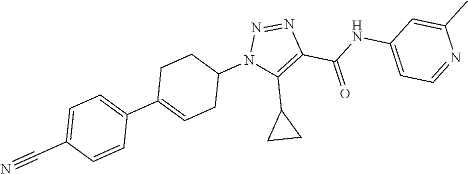 [SMILES: Cc1cc(NC(=O)c2nnn(C3CC=C(c4ccc(C#N)cc4)CC3)c2C2CC2)ccn1] |
| 24-1 | 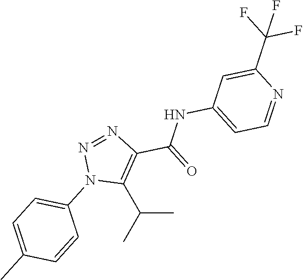 [SMILES: Cc1ccc(-n2nnc(C(=O)Nc3ccnc(C(F)(F)F)c3)c2C(C)C)cc1] |
| 25-1 | 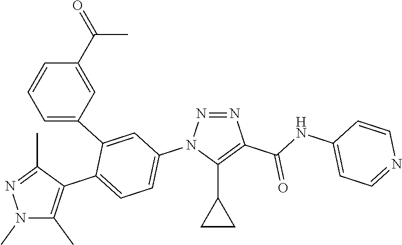 [SMILES: CC(=O)c1cccc(-c2cc(-n3nnc(C(=O)Nc4ccncc4)c3C3CC3)ccc2-c2c(C)nn(C)c2C)c1] |
| 25-2 | 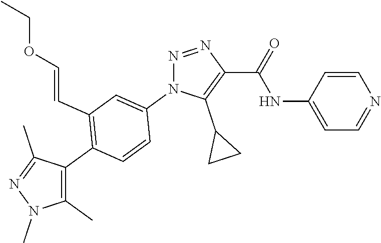 [SMILES: CCO/C=C/c1cc(-n2nnc(C(=O)Nc3ccncc3)c2C2CC2)ccc1-c1c(C)nn(C)c1C] |

All reagents, for which the synthesis is not described in the experimental part, are either commercially available, or are known compounds or may be formed from known compounds by known methods by a person skilled in the art. The compounds and intermediates produced according to the methods of the invention may require purification. Purification of organic compounds is well known to a person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using purpose-made or prepacked silica gel cartridges and eluents such as gradients of solvents such as heptane, ether, ethyl acetate, acetonitrile, ethanol and the like. In some cases, the compounds may be purified by preparative HPLC using methods as described.

Purification methods as described herein may provide compounds of the present invention which possess a sufficiently basic or acidic functionality in the form of a salt, such as, in the case of a compound of the present invention which is sufficiently basic, a trifluoroacetate or formate salt, or, in the case of a compound of the present invention, which is sufficiently acidic, an ammonium salt. A salt of this type can either be transformed into its free base or free acid form, respectively, by various methods known to a person skilled in the art or be used as salts in subsequent biological assays. It is to be understood that the specific form of a compound of the present invention as isolated and as described herein is not necessarily the only form in which said compound can be applied to a biological assay in order to quantify the specific biological activity.

Chemical names were generated using the naming function in ChemDraw software (Version 19.0.1.28) by PerkinElmer Informatics, Inc. In some cases, generally accepted names of commercially available reagents were used in place of names generated by the naming software.

### EXAMPLES

### General Methods

¹H NMR (400 MHz) were obtained in solution of deuteriochloroform (CDCl₃), deuteriomethanol (CD₃OD) or dimethyl sulfoxide—D₆ (DMSO-D₆).

HPLC retention times, purities, and mass spectra (LCMS) were obtained using the following methods:

Method 1: Agilent 1260 Infinity II System equipped with an Agilent Poroshell 120 EC-18, 2.7 μm, 4.6×100 mm column at 30° C., using H₂O with 0.1% FA as the mobile phase A, and CH₃CN with 0.1% FA as the mobile phase B. An ESI detector in positive mode was used. The gradient was 5-95% mobile phase B over 12 min then held at 95% for 1.8 min, then returned to 10% mobile phase B over 0.2 min The flow rate was 1 mL/min.

Method 2: Shimadzu SCL-10A system equipped with Agilent Eclipse XDB-C18, 3.5 μM, 4.6×150 mm column and PE Sciex API 150 EX, using H₂O with 0.1% TFA as the mobile phase A, and MeOH with 0.1% TFA as the mobile phase B. The gradient was 5-95% mobile phase B over 12 min then held at 95% mobile phase B for 3 min, then returned to 5% mobile phase B for 1 min. The flow rate was 1 mL/min.

Method 3: Shimadzu SCL-10A system equipped with Agilent Eclipse XDB-C18, 3.5 μM, 4.6×150 mm column and PE Sciex API 150 EX, using H₂O with 0.1% TFA as the mobile phase A, and MeOH with 0.1% TFA as the mobile phase B. The gradient was 50-95% mobile phase B over 4 min then held at 95% mobile phase B for 4 min, then returned to 50% mobile phase B for 0.1 min. The flow rate was 1 mL/min.

Method 4: SHIMADZU LCMS-2020 System equipped with a Kinetex EVO C18 2.1×30 mm, (5 μm particles), using H₂O with 0.0375% TFA as the mobile phase A, and CH₃CN with 0.01875% TFA as the mobile phase B. An ESI detector in positive mode was used. The gradient was 5% B at 0.00 min and 5-90% B at 0.00-0.80 min, 90-95% B at 0.80-1.12 min, and then 95-5% B in 0.01 min, hold on 5% B for 0.34 min, the flow rate was 1.5 ml/min.

Method 5: SHIMADZU LCMS-2020 System equipped with a Kinetex EVO C18 2.1×30 mm, (5 μm particles), using H₂O with 0.025% NH₃·H₂O as the mobile phase A, and CH₃CN as the mobile phase B. An ESI detector in positive mode was used. The gradient was 5% B at 0.00 min and 5-95% B at 0.00-1.2 min, 95-5% B at 1.20-1.21 min, hold on 5% B for 0.34 min, the flow rate was 1.5 ml/min.

Method 6: SHIMADZU LCMS-2020 System equipped with a Kinetex EVO C18 2.1×30 mm, (5 μm particles), using H₂O with 0.0375% TFA as the mobile phase A, and CH₃CN with 0.01875% TFA as the mobile phase B. An ESI detector in positive mode was used. The gradient was 0% B at 0.00 min and 0-60% B at 0.00-0.80 min, 60-0% B at 0.80-1.20 min, hold on 0% B for 0.34 min, the flow rate was 1.5 ml/min.

Method 7: LCMS-2020 System equipped with a HALO C18 3.0×30 mm, (2.7 μm particles), using H₂O with 0.0375% TFA as the mobile phase A, and CH₃CN with 0.01875% TFA as the mobile phase B. An ESI detector in positive mode was used. The gradient was 5% B at 0.00 min and 5-95% B at 0.00-0.50 min, held on 95% B for 0.30 min, 95-5% B at 0.80-0.81 min, and then held on 5% B for 0.24 min, the flow rate was 1.5 ml/min.

The pyridine, dichloromethane (DCM), tetrahydrofuran (THF), acetonitrile, DMF, and toluene used in the procedures were from Aldrich Sure-Seal bottles, or similar, and kept under nitrogen (N₂). All reactions were stirred magnetically, and temperatures are external reaction temperatures. Chromatographies were typically carried out using a Combiflash Rf flash purification system (Teledyne Isco) equipped with Redisep (Teledyne Isco) silica gel (SiO₂) columns or by using a similar system.

Preparative HPLC purifications were typically performed using one of the following systems or similar: 1) Waters System equipped with a Waters 2489 uv/vis detector, an Aquity QDA detector, a Waters xBridge Prep C18 5 uM OBD, 30×1560 mm column, and eluting with various gradients of H₂O/CH₃CN (0.1% FA) at a 30 mL/min flow rate, or 2) column: Phenomenex Synergi C18 150×30 mm-4 μm; mobile phase: [H₂O (0.225% FA)-CH₃CN]; B %: 55%-85%, 12 min) and desired fractions were typically concentrated using a Genevac EZ-2.

The following abbreviations are used: ethyl acetate (EA), triethylamine (TEA), dimethylformamide (DMF), diisopropylethylamine (DIEA), dichloromethane (DCM), methanol (MeOH), petroleum ether (pet ether), tetrahydrofuran (THF), (1-[Bis(dimethylamino)methylene]-1H-1,2,3-triazolo[4,5-b]pyridinium 3-oxide hexafluorophosphate (HATU), dimethylsufoxide (DMSO), dichloroethane (DCE), liquid chromatography-mass spectrometer (LCMS), nuclear magnetic resonance (NMR), deuterated chloroform (CDCl₃), trifluoroacetic acid (TFA), reverse phase (RP), hour (h), minute (min), 1,8-Diazabicyclo[5.4.0]undec-7-ene (DBU), p-toluenesulfonyl azide (N₃Ts), Hertz (Hz), retention time (t_{R}), trimethylsilyl azide (TMSN₃), petroleum ether (pet ether), acetonitrile (CH₃CN), intermediate (INT), 1,1′-Bis(diphenylphosphino)ferrocene (dppf), retention factor (R_{f}), formic acid (FA), water (H₂O). Other abbreviations commonly known to the art may be included.

### Example 1

### Synthesis of Compound 1-4 and Other Representative Compounds

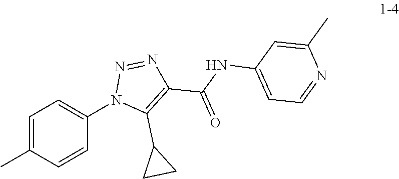

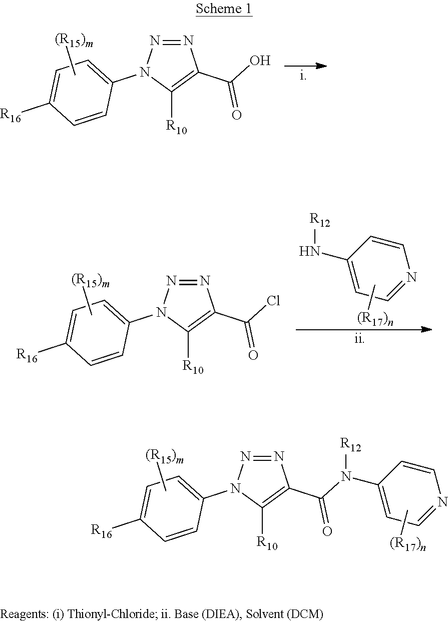

### Step 1-1. 5-cyclopropyl-N-(2-methylpyridin-4-yl)-1-(p-tolyl)-1H-1,2,3-triazole-4-carboxamide (Compound 1-4)

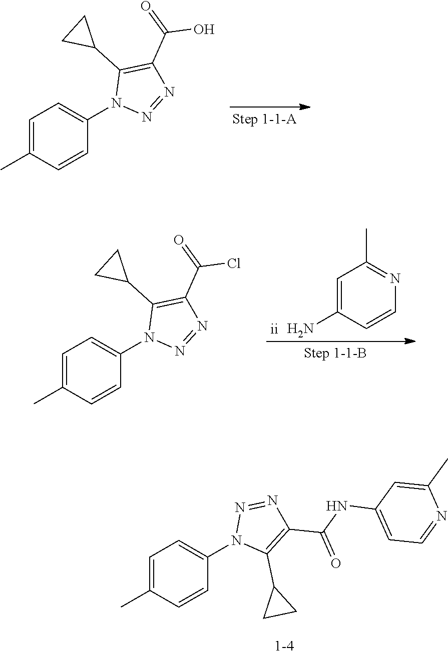

A sample of 5-cyclopropyl-1-(p-tolyl)-1H-1,2,3-triazole-4-carboxylic acid (250 mg, 1.02 mmol) was dissolved in thionyl chloride (5 mL). After the mixture was heated at 75° C. for 2 h, it was concentrated to remove excess thionyl chloride. The resulting residue was dissolved in DCM (5 mL) and then DIEA (132 mg, 1.02 mmol) and 2-methylpyridin-4-amine (122 mg, 1.13 mmol) were added. After stirring for 10 min, the reaction mixture was diluted with DCM and H₂O. The organic layer was collected, and the aqueous layer was extracted with DCM (2×). The resulting organic layers were combined, dried (Na₂SO₄), filtered, concentrated, and purified by SiO₂ chromatography (EA/hexanes) to afford product that was triturated with MeOH/H₂O to give 270 mg (97%) of 5-cyclopropyl-N-(2-methylpyridin-4-yl)-1-(p-tolyl)-1H-1,2,3-triazole-4-carboxamide (Compound 1-4.) LCMS-ESI (m/z) calculated for C₁₉H₁₉N₅O: 333.4; found 334.4 [M+H]⁺, t_{R}=10.89 min (Method 2). ¹H NMR (400 MHz, CDCl₃) δ 9.32 (s, 1H), 8.45 (d, J=8.0 Hz, 1H), 7.64 (s, 1H), 7.39-7.47 (m, 5H), 2.62 (s, 3H), 2.51 (s, 3H), 1.99 (m, 1H), 1.18 (d, J=8.0 Hz, 2H), 1.03 (d, J=8.0 Hz, 2H).

The compounds listed in Table 1 were made using the procedures of Scheme 1.

TABLE 1

| | | Purity | | | | |
|---|---|---|---|---|---|---|
| | Cpd | RT | | Observed | | Purity |
| Structure | No | (min) | MW | m/z | Ion | Method |
| 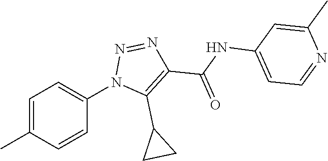 | 1-4 | 10.89 | 333.4 | 334.4 | [M + H]⁺ | 2 |
| 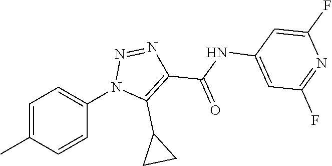 [SMILES: Cc1ccc(-n2nnc(C(=O)Nc3cc(F)nc(F)c3)c2C2CC2)cc1] | 1-5 | 13.85 | 355.3 | 356.1 | [M + H]⁺ | 2 |
| 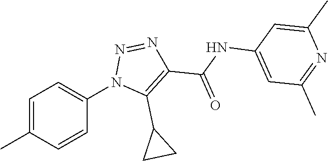 | 1-6 | 11.21 | 347.4 | 348.4 | [M + H]⁺ | 2 |
| 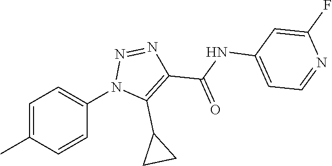 [SMILES: Cc1ccc(-n2nnc(C(=O)Nc3ccnc(F)c3)c2C2CC2)cc1] | 1-7 | 12.2 | 337.4 | 338.4 | [M + H]⁺ | 2 |
| 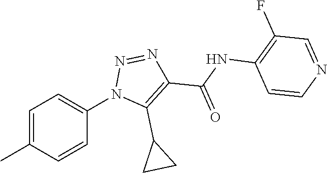 [SMILES: Cc1ccc(-n2nnc(C(=O)Nc3ccncc3F)c2C2CC2)cc1] | 1-9 | 11.46 | 337.4 | 338.1 | [M + H]⁺ | 2 |
| 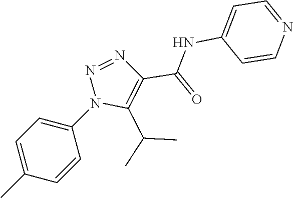 [SMILES: Cc1ccc(-n2nnc(C(=O)Nc3ccncc3)c2C(C)C)cc1] | 1-19 | 10.52 | 321.4 | 322.5 | [M + H]⁺ | 2 |

### Example 2

### Synthesis of Compound 2-4 and Other Representative Compounds

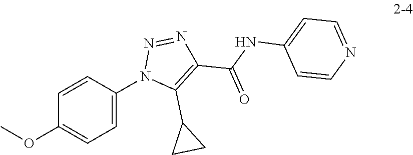 [SMILES: COc1ccc(-n2nnc(C(=O)Nc3ccncc3)c2C2CC2)cc1]

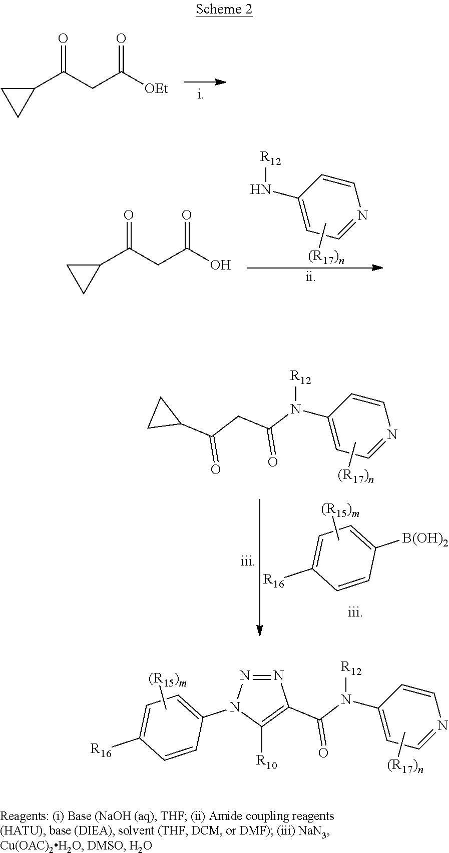

### Step 2-1. Synthesis of 3-cyclopropyl-3-oxopropanoic acid (INT 2A)

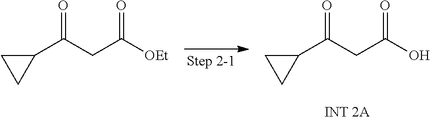 [SMILES: CCOC(=O)CC(=O)C1CC1 ; O=C(O)CC(=O)C1CC1]

To a stirring solution of ethyl 3-cyclopropyl-3-oxopropanoate (1 g, 6.4 mmol) in THF (20 mL) was added a solution of 1M aqueous NaOH (12.8 mL, 12.8 mmol). After 12 h, the reaction mixture was concentrated to provide 820 mg (99%) of crude 3-cyclopropyl-3-oxopropanoic acid (INT 2A) that was used without further purification.

### Step 2-2. Synthesis of 3-cyclopropyl-3-oxo-N-(pyridin-4-yl)propanamide (INT 2B)

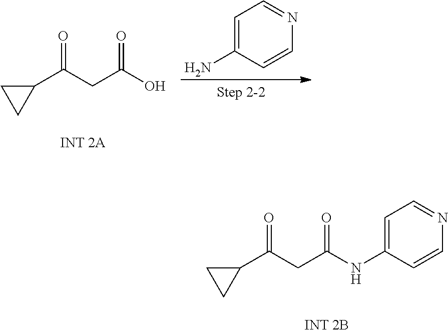 [SMILES: O=C(O)CC(=O)C1CC1 ; Nc1ccncc1 ; O=C(CC(=O)C1CC1)Nc1ccncc1]

To a round bottom flask containing stirring solution of INT 2A (300 mg, 2.3 mmol) pyridin-4-amine (243 mg, 2.6 mmol), and HATU (934 mg, 2.50 mmol) were added DCM (7 mL) and DIEA (820 μL, 4.7 mmol). The reaction mixture was stirred for 2 h, diluted with EA, washed with H₂O, sat aq. NaHCO₃ and brine, then dried (Na₂SO₄), filtered and concentrated to provide crude material that was purified by SiO₂ chromatography (EA/hexane) to afford 400 mg (84.0%) of 3-cyclopropyl-3-oxo-N-(pyridin-4-yl) propanamide (INT 2B). LCMS (m/z) calculated for C₁₁H₁₂N₂O₂: 204.1; found 205.2 [M+H]⁺ (Method 3). ¹H NMR (400 MHz, CDCl₃) δ 9.87 (s, 1H), 8.51 (d, J=4.0 Hz, 2H), 7.52 (d, J=4.0 Hz, 2H), 3.77 (s, 2H), 2.03-2.09 (m, 1H), 1.17-1.25 (m, 2H), 1.09-1.14 (m, 2H).

### Step 2-3. Synthesis of 5-cyclopropyl-1-(4-methoxyphenyl)-N-(pyridin-4-yl)-1H-1,2,3-triazole-4-carboxamide (Compound 2-4)

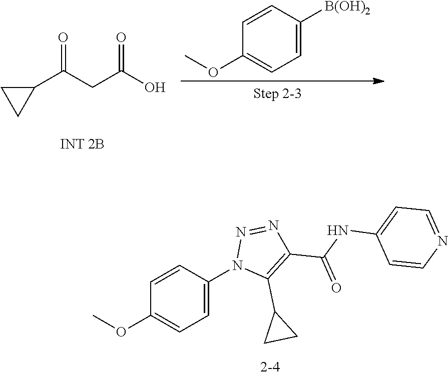 [SMILES: O=C(O)CC(=O)C1CC1 ; COc1ccc(B(O)O)cc1 ; COc1ccc(-n2nnc(C(=O)Nc3ccncc3)c2C2CC2)cc1]

To a stirring solution of (4-methoxyphenyl) boronic acid (56 mg, 0.37 mmol) in DMSO (3 mL) were added sodium azide (47.9 mg, 0.74 mmol), H₂O (1 mL), and Cu(OAc)₂ (H₂O)₂ (7.4 mg, 0.04 mmol). After stirring for 2 h, INT 2B (75 mg, 0.37 mmol) and pyrrolidine (˜0.1 mL) were added. After stirring further for 12 h, the reaction mixture was diluted with EA and washed consecutively with sat NaHCO₃, H₂O, and brine. The aqueous layer was dried (Na₂SO₄), filtered, concentrated, and purified by SiO₂ chromatography (EA/hex). The resulting material was further purified by reverse-phase chromatography (MeOH/H₂O) to afford 4.3 mg (3%) of 5-cyclopropyl-1-(4-methoxyphenyl)-N-(pyridin-4-yl)-1H-1,2,3-triazole-4-carboxamide (Compound 2-4). LCMS-ESI (m/z) calculated for C₁₈H₁₇N₅O₂: 335.4, found 336.5 [M+H]⁺, t_{R}=8.97 min (Method 2). ¹H NMR (400 MHz, DMSO-D₆) δ 10.06 (s, 1H), 8.73 (s, 2H), 8.21 (s, 2H), 7.48 (d, J=8.0 Hz, 2H), 7.11 (d, J=8.0 Hz, 2H), 3.94 (s, 3H), 2.01 (s, 1H), 1.22 (d, J=8.0 Hz, 2H), 1.12 (d, J=8.0 Hz, 2H).

The compounds listed in Table 2 were made using the procedures of Scheme 2.

TABLE 2

| | | Purity | | | | |
|---|---|---|---|---|---|---|
| | Cpd | RT | | Observed | | Purity |
| Structure | No. | (min) | MW | m/z | Ion | Method |
| 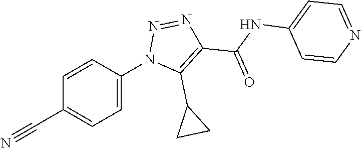 [SMILES: N#Cc1ccc(-n2nnc(C(=O)Nc3ccncc3)c2C2CC2)cc1] | 2-1 | 8.25 | 330.4 | 331.2 | [M + H]⁺ | 2 |
| 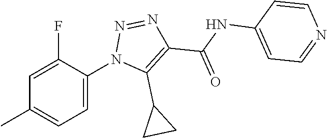 [SMILES: Cc1ccc(-n2nnc(C(=O)Nc3ccncc3)c2C2CC2)c(F)c1] | 2-2 | 9.51 | 337.4 | 338.2 | [M + H]⁺ | 2 |
| 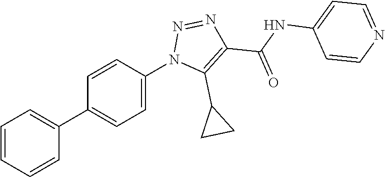 [SMILES: O=C(Nc1ccncc1)c1nnn(-c2ccc(-c3ccccc3)cc2)c1C1CC1] | 2-3 | 11.28 | 381.4 | 382.3 | [M + H]⁺ | 2 |
| 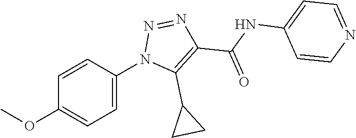 [SMILES: COc1ccc(-n2nnc(C(=O)Nc3ccncc3)c2C2CC2)cc1] | 2-4 | 9.51 | 335.4 | 336.5 | [M + H]⁺ | 2 |
| 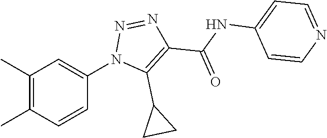 [SMILES: Cc1ccc(-n2nnc(C(=O)Nc3ccncc3)c2C2CC2)cc1C] | 2-5 | 11.24 | 333.4 | 334.4 | [M + H]⁺ | 2 |
| 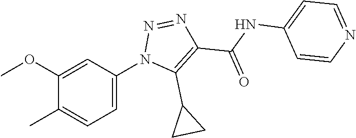 [SMILES: COc1cc(-n2nnc(C(=O)Nc3ccncc3)c2C2CC2)ccc1C] | 2-6 | 10.72 | 349.4 | 350.6 | [M + H]⁺ | 2 |
| 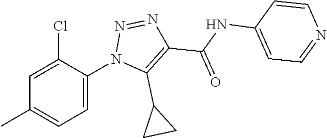 [SMILES: Cc1ccc(-n2nnc(C(=O)Nc3ccncc3)c2C2CC2)c(Cl)c1] | 2-7 | 10.83 | 353.8 | 354.4 | [M + H]⁺ | 2 |
| 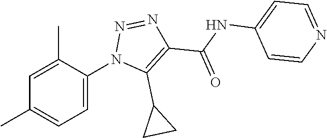 [SMILES: Cc1ccc(-n2nnc(C(=O)Nc3ccncc3)c2C2CC2)c(C)c1] | 2-8 | 11.12 | 333.4 | 334.4 | [M + H]⁺ | 2 |
| 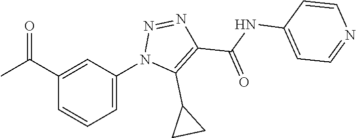 [SMILES: CC(=O)c1cccc(-n2nnc(C(=O)Nc3ccncc3)c2C2CC2)c1] | 2-9 | 9.4 | 347.4 | 348.5 | [M + H]⁺ | 2 |
| 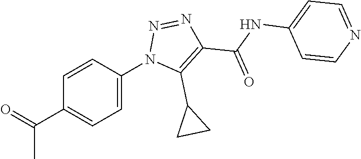 [SMILES: CC(=O)c1ccc(-n2nnc(C(=O)Nc3ccncc3)c2C2CC2)cc1] | 2-10 | 8.61 | 347.4 | 348.4 | [M + H]⁺ | 2 |
| 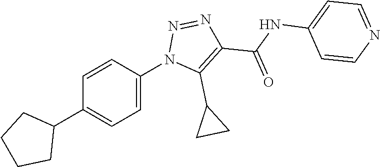 [SMILES: O=C(Nc1ccncc1)c1nnn(-c2ccc(C3CCCC3)cc2)c1C1CC1] | 2-11 | 12.89 | 373.5 | 374.3 | [M + H]⁺ | 2 |
| 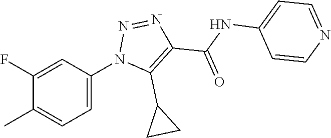 [SMILES: Cc1ccc(-n2nnc(C(=O)Nc3ccncc3)c2C2CC2)cc1F] | 2-13 | 10.1 | 337.4 | 338.4 | [M + H]⁺ | 2 |
| 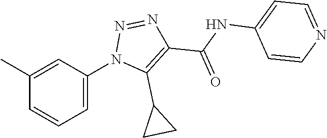 [SMILES: Cc1cccc(-n2nnc(C(=O)Nc3ccncc3)c2C2CC2)c1] | 2-14 | 9.94 | 319.4 | 320.3 | [M + H]⁺ | 2 |

### Example 3

### Synthesis of Compound 3-1 and Other Representative Compounds

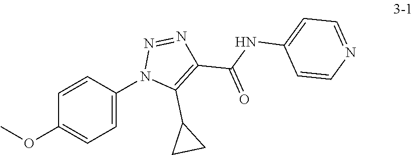 [SMILES: COc1ccc(-n2nnc(C(=O)Nc3ccncc3)c2C2CC2)cc1]

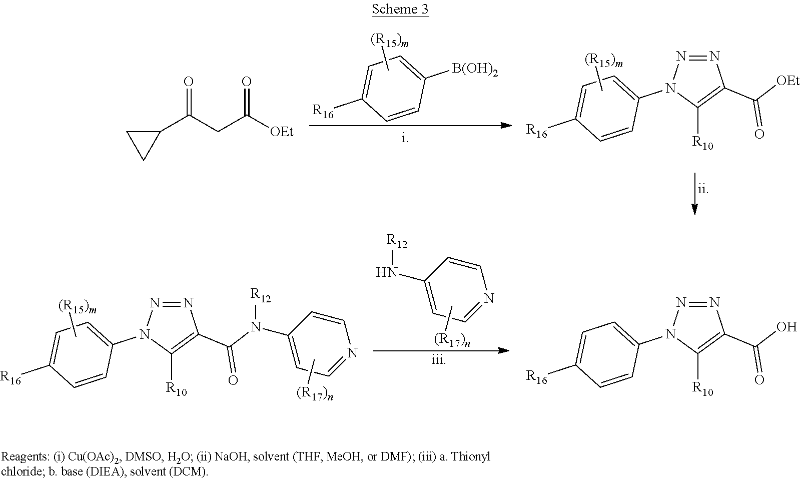

### Step 3-1. Synthesis of ethyl 5-cyclopropyl-1-(3-ethylphenyl)-1H-1,2,3-triazole-4-carboxylate (INT 3A)

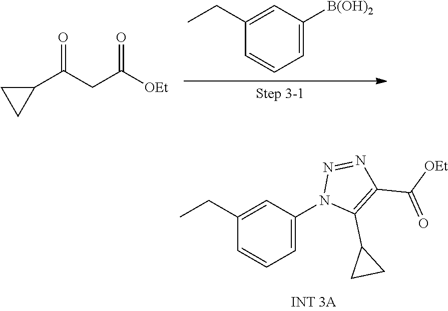 [SMILES: CCOC(=O)CC(=O)C1CC1 ; CCc1cccc(B(O)O)c1 ; CCOC(=O)c1nnn(-c2cccc(CC)c2)c1C1CC1]

To a stirring solution of (3-ethylphenyl)boronic acid (100 mg, 0.67 mmol) in DMSO (5 mL) and H₂O (1 mL) were added Cu(OAc)₂ (12 mg, 0.067 mmol) and sodium azide (86.7 mg, 1.3 mmol). After stirring for 2 h, ethyl 3-cyclopropyl-3-oxopropanoate (104.1 mg, 0.67 mmol) and pyrrolidine (9.6 mg, 0.13 mmol) were added. The reaction mixture was stirred at rt overnight then diluted with EA and washed with H₂O and brine, then dried (Na₂SO₄), filtered, concentrated, and purified by SiO₂ chromatography (MeOH/DCM) to provide 12.9 mg (7%) of ethyl 5-cyclopropyl-1-(3-ethylphenyl)-1H-1,2,3-triazole-4-carboxylate (INT 3A). LCMS-ESI (m/z) calculated for C₁₆H₁₉N₃O₂: 285.3; found 286.5 [M+H]⁺, t_{R}=5.65 min (Method 3).

### Step 3-2. Synthesis of 5-cyclopropyl-1-(3-ethylphenyl)-1H-1,2,3-triazole-4-carboxylic acid (INT 3B)

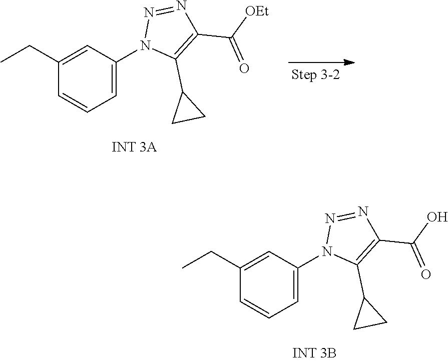 [SMILES: CCOC(=O)c1nnn(-c2cccc(CC)c2)c1C1CC1 ; CCc1cccc(-n2nnc(C(=O)O)c2C2CC2)c1]

To a stirring solution of INT 3A (12.9 mg, 0.04 mmol) in EtOH (3 mL) was added a solution of NaOH (9 mg, 0.22 mmol) in H₂O (1 mL). The mixture was stirred for 12 h at 80° C. then diluted with H₂O and washed with EA (50 mL). The aqueous layer was acidified to pH 1-2 by the addition of 1 N HCl and then extracted with EA (3×). The combined organic layers were washed with brine, dried (Na₂SO₄), filtered and concentrated to provide 11.6 mg (99%) of 5-cyclopropyl-1-(3-ethylphenyl)-1H-1,2,3-triazole-4-carboxylic acid (INT 3B) as a white solid. LCMS-ESI (m/z) calculated for C₁₄H₁₅N₅O₂: 257.3; found 258.3 [M+H]⁺, t_{R}=3.99 min (Method 3).

### Step 3-3. Synthesis of 5-cyclopropyl-1-(3-ethylphenyl)-N-(pyridin-4-yl)-1H-1,2,3-triazole-4-carboxamide (3-1)

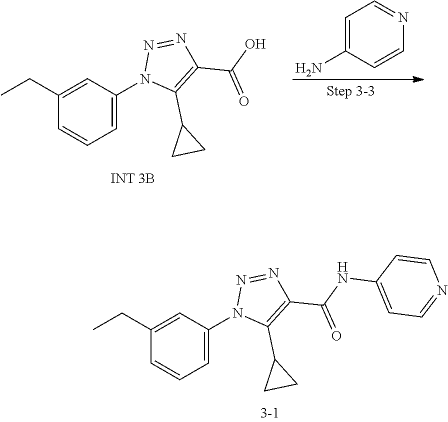 [SMILES: CCc1cccc(-n2nnc(C(=O)O)c2C2CC2)c1 ; Nc1ccncc1 ; CCc1cccc(-n2nnc(C(=O)Nc3ccncc3)c2C2CC2)c1]

A stirring solution of INT 3B (11.6 mg, 0.04 mmol) in thionyl chloride (1 mL) was refluxed at 75° C. for 1 h under an atmosphere of N₂. The mixture was cooled and concentrated in vacuo. The resulting residue was dissolved in anhydrous DCM (2 mL) and DIEA (7.0 mg, 0.05 mml) and pyridin-4-amine (4.2 mg, 0.05 mmol) were added. After stirring for 2 h, the mixture was washed with H₂O and brine, dried (Na₂SO₄), concentrated, and purified by SiO₂ chromatography (EA/hex) to provide 5 mg (33%) of 5-cyclopropyl-1-(3-ethylphenyl)-N-(pyridin-4-yl)-1H-1,2,3-triazole-4-carboxamide (3-1) as a white solid. LCMS-ESI (m/z) calculated for C₁₉H₁₉N₅O: 333.2; found 334.4 [M+H]⁺, t_{R}=9.94 min (Method 2). ¹H NMR (400 MHz, CDCl₃) δ 9.97 (s, 1H), 8.62 (s, 2H), 8.14 (s, 2H), 7.54 (t, J=8.0 Hz, 1H), 7.46 (d, J=8.0 Hz, 1H), 7.36-7.40 (m, 2H), 2.81 (q, J=8.0 Hz, 2H), 2.02 (quint, J=4.0 H, 1H), 1.34 (t, J=8.0 Hz, 3H), 1.17-1.28 (m, 2H), 1.09-1.14 (m, 2H).

The compounds listed in Table 3 were made using the procedures of Scheme 3.

TABLE 3

| | | Purity | | | | |
|---|---|---|---|---|---|---|
| | Cpd | RT | | Observed | | Purity |
| Structure | No. | (min) | MW | m/z | Ion | Method |
| 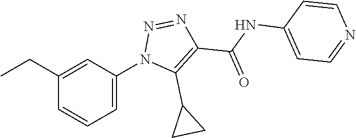 [SMILES: CCc1cccc(-n2nnc(C(=O)Nc3ccncc3)c2C2CC2)c1] | 3-1 | 10.31 | 333.4 | 334.4 | [M + H]⁺ | 2 |
| 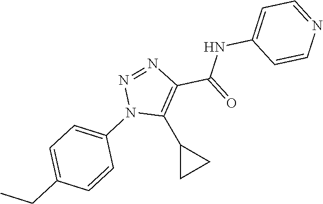 [SMILES: CCc1ccc(-n2nnc(C(=O)Nc3ccncc3)c2C2CC2)cc1] | 3-2 | 10.7 | 333.4 | 334.4 | [M + H]⁺ | 2 |
| 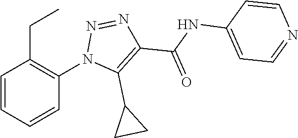 [SMILES: CCc1ccccc1-n1nnc(C(=O)Nc2ccncc2)c1C1CC1] | 3-3 | 10.86 | 333.4 | 334.4 | [M + H]⁺ | 2 |
| 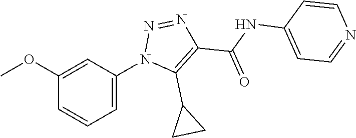 [SMILES: COc1cccc(-n2nnc(C(=O)Nc3ccncc3)c2C2CC2)c1] | 3-4 | 9.48 | 335.4 | 336.2 | [M + H]⁺ | 2 |
| 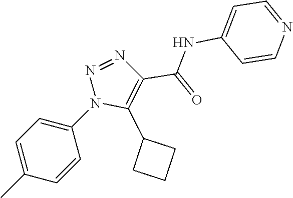 [SMILES: Cc1ccc(-n2nnc(C(=O)Nc3ccncc3)c2C2CCC2)cc1] | 3-5 | 10.85 | 333.4 | 334.4 | [M + H]⁺ | 2 |
| 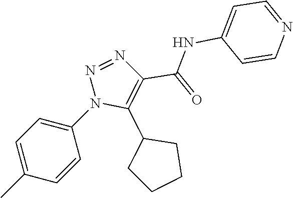 [SMILES: Cc1ccc(-n2nnc(C(=O)Nc3ccncc3)c2C2CCCC2)cc1] | 3-6 | 11.42 | 347.4 | 348.4 | [M + H]⁺ | 2 |

### Example 4

### Synthesis of Compound 4-2 and Other Representative Compounds

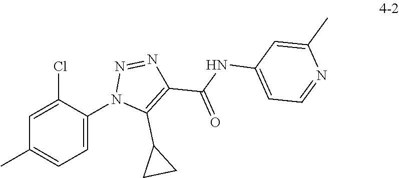

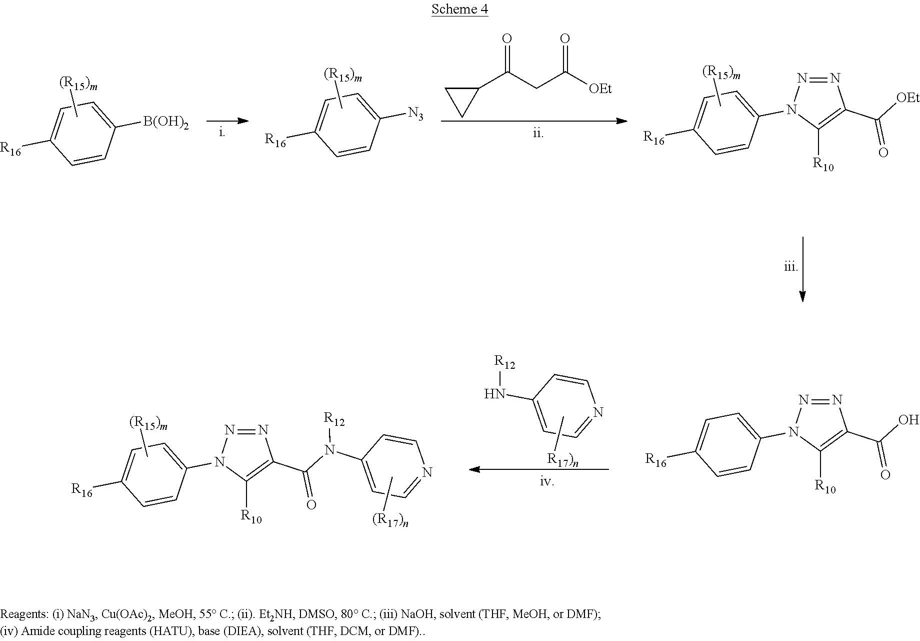

### Step 4-1. Synthesis of 1-azido-2-chloro-4-methylbenzene (INT 4A)

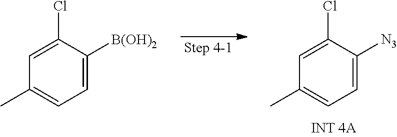

To a solution of (2-chloro-4-methylphenyl)boronic acid (800 mg, 4.7 mmol) in MeOH (30 mL) were added NaN₃ (610 mg, 9.4 mmol) and Cu(OAc)₂ (85 mg, 0.47 mmol). After stirring at 55° C. for 3 h, the reaction mixture was partially concentrated, diluted with EA, and washed with H₂O. The organic layer was dried (Na₂SO₄), filtered, and partially concentrated to provide crude 1-azido-2-chloro-4-methylbenzene (INT 4A) that was used without further purification.

### Step 4-2. Synthesis of ethyl 1-(2-chloro-4-methylphenyl)-5-cyclopropyl-1H-1,2,3-triazole-4-carboxylate (INT 4B)

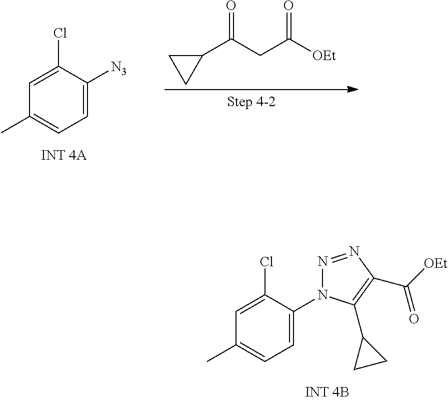 [SMILES: Cc1ccc(N=[N+]=[N-])c(Cl)c1 ; CCOC(=O)CC(=O)C1CC1 ; CCOC(=O)c1nnn(-c2ccc(C)cc2Cl)c1C1CC1]

To a solution of INT 4A (335 mg, 2.0 mmol) in DMSO (3 mL) were added ethyl 3-cyclopropyl-3-oxopropanoate (218 mg, 1.4 mmol) and Et₂NH (80 mg, 1.1 mmol). After stirring at 80° C. for 12 h, the reaction mixture was purified by reverse-phase SiO₂ chromatography (MeOH/H₂O) to provide 119 mg (28%) of ethyl 1-(2-chloro-4-methylphenyl)-5-cyclopropyl-1H-1,2,3-triazole-4-carboxylate (INT 4B). LCMS-ESI (m/z) calculated for C₁₅H₁₆ClN₃O₂: 305.1; found 306.2 [M+H]⁺, t_{R}=6.27 min (Method 3).

### Step 4-3. Synthesis of 1-(2-chloro-4-methylphenyl-5-cyclopropyl-1H-1,2,3-triazole-4-carboxylic acid (INT 4C)

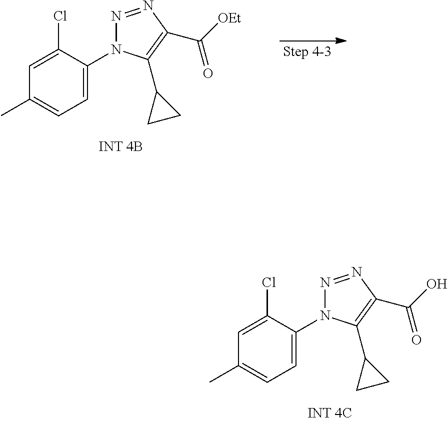 [SMILES: CCOC(=O)c1nnn(-c2ccc(C)cc2Cl)c1C1CC1 ; Cc1ccc(-n2nnc(C(=O)O)c2C2CC2)c(Cl)c1]

To a solution of INT 4B (120 mg, 2.0 mmol) in EtOH (3 mL) was added a solution of NaOH (78 mg, 2.0 mmol) in H₂O (1 mL). After stirring at 78° C. for 2 h, the reaction mixture was concentrated, diluted with H₂O, and washed with EA. The pH of the aqueous layer was adjusted to pH 1-2 by the addition of 1N HCl and was then extracted with EA (3×). The combined organic layers were washed with brine, dried (Na₂SO₄), filtered, and concentrated to provide 108 mg (99%) of 1-(2-chloro-4-methylphenyl)-5-cyclopropyl-1H-1,2,3-triazole-4-carboxylic acid (INT 4C) as a white solid. LCMS-ESI (m/z) calculated for C₁₃H₁₂ClN₃O₂: 277.1; found 278.0 [M+H]⁺, t_{R}=6.04 min (Method 3).

### Step 4-4. Synthesis of 1-(2-chloro-4-methylphenyl)-5-cyclopropyl-N-(2-methylpyridin-4-yl)-1H-1,2,3-triazole-4-carboxamide (INT 4-2)

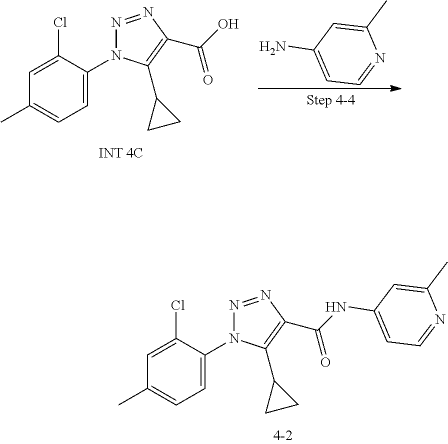

To a solution of INT 4C (50 mg, 0.2 mmol) in DMF (1 mL) was added HATU (75.3 mg, 0.2 mmol). After stirring for 5 min, 2-methylpyridin-4-amine (20 mg, 0.2 mmol) and DIEA (95 mg, 0.74 mmol) were added. After stirring for 12 h, the reaction mixture was treated with 2-3 drops of TFA and directly purified by reverse phase-SiO₂ chromatography (MeOH/H₂O with 0.1% TFA). The fractions with clean product were combined, washed with 1 M NaOH (to remove residual TFA) and concentrated to provide 26 mg (39%) of 1-(2-chloro-4-methylphenyl)-5-cyclopropyl-N-(2-methylpyridin-4-yl)-1H-1,2,3-triazole-4-carboxamide (4-2) as a white solid. LCMS-ESI (m/z) calculated for C₁₉H₁₈ClN₅O: 367.1, found 368.1 [M+H]⁺, t_{R}=11.13 min (Method 2). ¹H NMR (400 MHz, CDCl₃) δ 9.54 (s, 1H), 8.47 (d, J=8.0 Hz, 1H), 7.78 (s, 1H), 7.63 (s, 1H), 7.49 (s, 1H), 7.39 (d, J=8.0 Hz, 1H), 7.33 (d, J=8.0 Hz, 1H), 2.75 (s, 3H), 2.51 (s, 3H), 1.91 (m, 1H), 1.25 (d, J=8.0 Hz, 2H), 1.03 (d, J=8.0 Hz, 2H).

The compounds listed in Table 4 were made using the procedures of Scheme 4.

TABLE 4

| | | Purity | | | | |
|---|---|---|---|---|---|---|
| | Cpd | RT | | Observed | | Purity |
| Structure | No. | (min) | MW | m/z | Ion | Method |
| 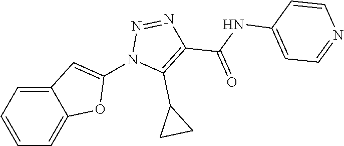 [SMILES: O=C(Nc1ccncc1)c1nnn(-c2cc3ccccc3o2)c1C1CC1] | 4-1 | 11.49 | 346 | 345.4 | [M + H]⁺ | 2 |
| 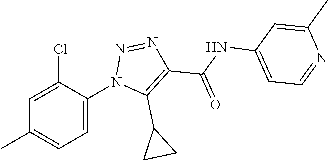 | 4-2 | 11.22 | 368.1 | 367.8 | [M + H]⁺ | 2 |
| 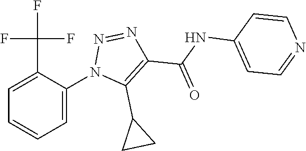 [SMILES: O=C(Nc1ccncc1)c1nnn(-c2ccccc2C(F)(F)F)c1C1CC1] | 4-3 | 10.37 | 374.2 | 373.3 | [M + H]⁺ | 2 |
| 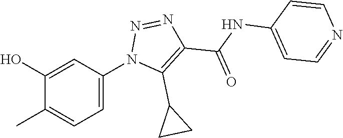 [SMILES: Cc1ccc(-n2nnc(C(=O)Nc3ccncc3)c2C2CC2)cc1O] | 4-4 | 10.39 | 336.3 | 335.4 | [M + H]⁺ | 2 |
| 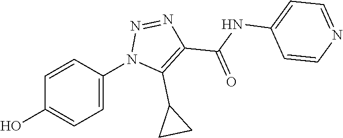 [SMILES: O=C(Nc1ccncc1)c1nnn(-c2ccc(O)cc2)c1C1CC1] | 4-5 | 9.01 | 322.5 | 321.3 | [M + H]⁺ | 2 |
| 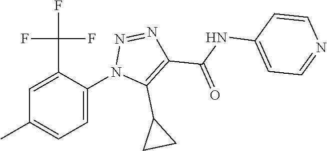 [SMILES: Cc1ccc(-n2nnc(C(=O)Nc3ccncc3)c2C2CC2)c(C(F)(F)F)c1] | 4-6 | 11.04 | 388.3 | 387.4 | [M + H]⁺ | 2 |
| 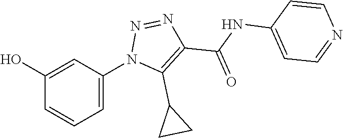 [SMILES: O=C(Nc1ccncc1)c1nnn(-c2cccc(O)c2)c1C1CC1] | 4-7 | 9.26 | 322.5 | 321.3 | [M + H]⁺ | 2 |
| 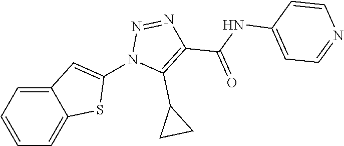 [SMILES: O=C(Nc1ccncc1)c1nnn(-c2cc3ccccc3s2)c1C1CC1] | 4-8 | 11.81 | 362.1 | 361.4 | [M + H]⁺ | 2 |
| 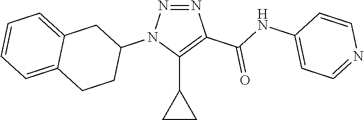 [SMILES: O=C(Nc1ccncc1)c1nnn(C2CCc3ccccc3C2)c1C1CC1] | 4-9 | 11.75 | 360.2 | 359.4 | [M + H]⁺ | 2 |
| 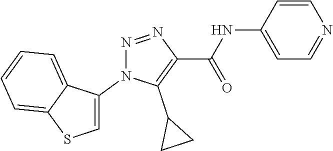 [SMILES: O=C(Nc1ccncc1)c1nnn(-c2csc3ccccc23)c1C1CC1] | 4-10 | 11.07 | 362.3 | 361.4 | [M + H]⁺ | 2 |
| 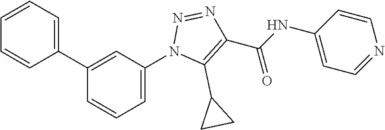 [SMILES: O=C(Nc1ccncc1)c1nnn(-c2cccc(-c3ccccc3)c2)c1C1CC1] | 4-11 | 11.14 | 382.3 | 381.4 | [M + H]⁺ | 2 |
| 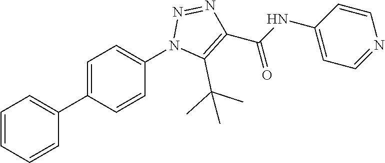 [SMILES: CC(C)(C)c1c(C(=O)Nc2ccncc2)nnn1-c1ccc(-c2ccccc2)cc1] | 4-12 | 12.49 | 398.2 | 397.5 | [M + H]⁺ | 2 |
| 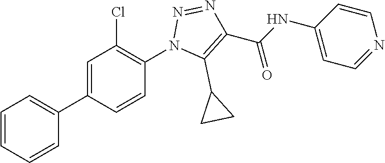 [SMILES: O=C(Nc1ccncc1)c1nnn(-c2ccc(-c3ccccc3)cc2Cl)c1C1CC1] | 4-13 | 12.51 | 415.8 | 415.9 | [M + H]⁺ | 2 |

### Example 5

### Synthesis of Compound 5-4 and Other Representative Compounds

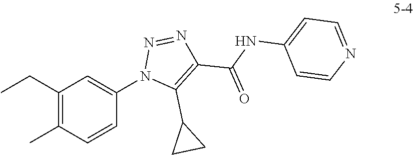 [SMILES: CCc1cc(-n2nnc(C(=O)Nc3ccncc3)c2C2CC2)ccc1C]

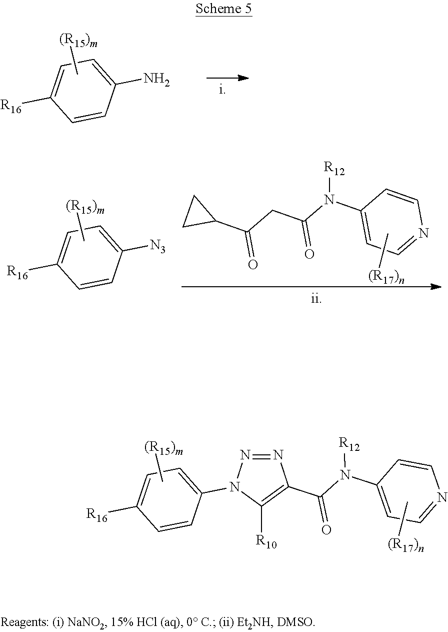

### Step 5-1. Synthesis of 4-azido-2-ethyl-1-methylbenzene (INT 5A)

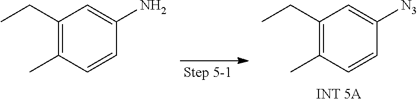

A solution of NaNO₂ (85.9 mg, 1.2 mmol) in H₂O (0.5 mL) was added dropwise to another solution of 3-ethyl-4-methylaniline (139 mg, 1.03 mmol) in 15% aqueous HCl (1 mL) at 0° C. After 30 min of stirring, a solution of NaN₃ (134.8 mg, 2.1 mmol) in H₂O (0.5 mL) was added dropwise at 0° C. After stirring for 1 h at 0° C., the reaction mixture was diluted with EA and washed with H₂O. The organic layer was dried (Na₂SO₄) and concentrated to provide 214 mg (99%) of crude INT 5A that was used without further purification.

### Step 5-2. Synthesis of 5-cyclopropyl-1-(3-ethyl-4-methylphenyl)-N-(pyridin-4-yl)-1H-1,2,3-triazole-4-carboxamide (Compound 5-4)

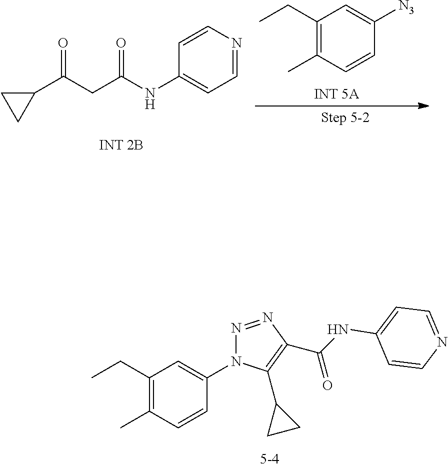

A solution of INT 2B (211 mg, 1.03 mmol), INT 5A (211 mg, 1.03 mmol) and Et₂NH (41 mg, 0.57 mmol) in DMSO (3 ml) was heated at 80° C. for 4 h. The mixture was purified by RP—SiO₂ chromatography (H₂O/MeOH, 0.1% TFA). The resulting clean fractions were combined, diluted in EA, washed with NaHCO₃ and concentrated to provide a residue that was further purified by SiO₂ chromatography (EA/hexanes) to afford 10 mg (14%) of 5-cyclopropyl-1-(3-ethyl-4-methylphenyl)-N-(pyridin-4-yl)-1H-1,2,3-triazole-4-carboxamide (Compound 5-4). LCMS-ESI (m/z) calculated for C₂₀H₂₁N₅O: 347.2; found 348.4 [M+H]⁺, t_{R}=10.6 min (Method 2) ¹H NMR (400 MHz, CDCl₃) δ=9.37 (s, 1H), 8.57 (s, 2H), 7.69 (s, 2H), 7.35-7.36 (m, 2H), 7.28-7.33 (m, 1H), 2.74 (q, J=8.0 Hz, 2H), 2.42 (s, 3H), 1.99-2.04 (m, 1H), 1.29 (t, J=8.0 Hz, 3H), 1.16-1.18 (m, 2H), 1.03-1.07 (m, 2H).

The compounds listed in Table 5 were made using the procedures of Scheme 5.

TABLE 5

| | | Purity | | | | |
|---|---|---|---|---|---|---|
| | Cpd | RT | | Observed | | Purity |
| Structure | No. | (min) | MW | m/z | Ion | Method |
| 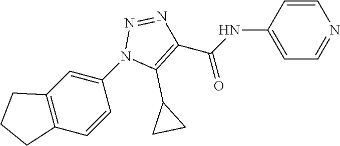 [SMILES: O=C(Nc1ccncc1)c1nnn(-c2ccc3c(c2)CCC3)c1C1CC1] | 5-1 | 11.59 | 345.4 | 346 | [M + H]⁺ | 2 |
| 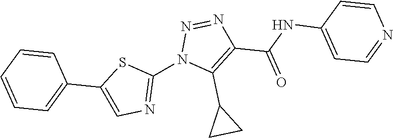 [SMILES: O=C(Nc1ccncc1)c1nnn(-c2ncc(-c3ccccc3)s2)c1C1CC1] | 5-2 | 12.43 | 388.4 | 389.2 | [M + H]⁺ | 2 |
| 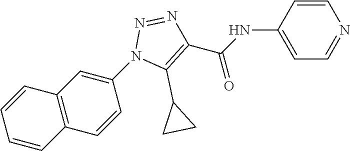 [SMILES: O=C(Nc1ccncc1)c1nnn(-c2ccc3ccccc3c2)c1C1CC1] | 5-3 | 11.46 | 355.4 | 356.2 | [M + H]⁺ | 2 |
| 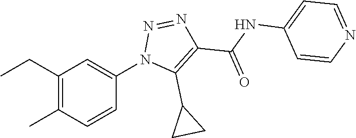 [SMILES: CCc1cc(-n2nnc(C(=O)Nc3ccncc3)c2C2CC2)ccc1C] | 5-4 | 11.7 | 347.4 | 347.4 | [M + H]⁺ | 2 |
| 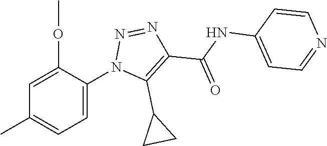 [SMILES: COc1cc(C)ccc1-n1nnc(C(=O)Nc2ccncc2)c1C1CC1] | 5-5 | 10.36 | 349.4 | 350.6 | [M + H]⁺ | 2 |
| 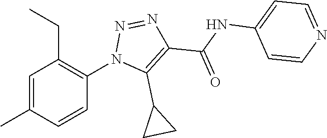 [SMILES: CCc1cc(C)ccc1-n1nnc(C(=O)Nc2ccncc2)c1C1CC1] | 5-6 | 11.39 | 347.4 | 348.2 | [M + H]⁺ | 2 |
| 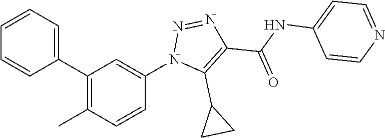 [SMILES: Cc1ccc(-n2nnc(C(=O)Nc3ccncc3)c2C2CC2)cc1-c1ccccc1] | 5-7 | 12.34 | 395.5 | 396.2 | [M + H]⁺ | 2 |

### Example 6

### Synthesis of Compound 6-2 and Other Representative Compounds

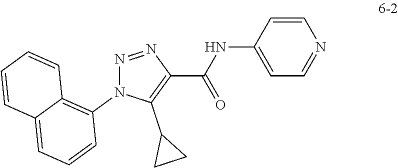 [SMILES: O=C(Nc1ccncc1)c1nnn(-c2cccc3ccccc23)c1C1CC1]

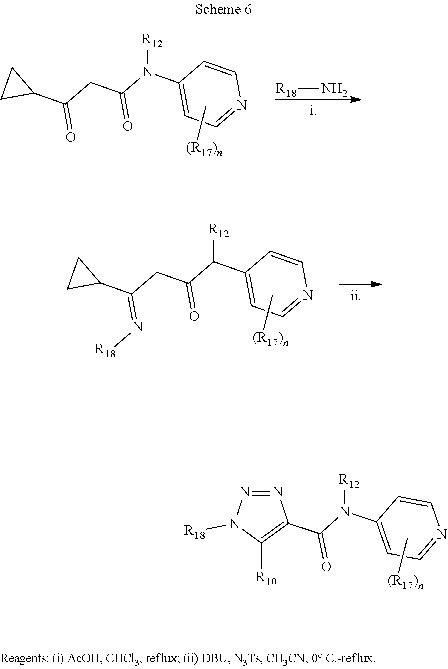

### Step 6-1. Synthesis of 3-cyclopropyl-3-(naphthalen-1-ylimino)-N-pyridin-4-yl)propanamide (INT 6-A)

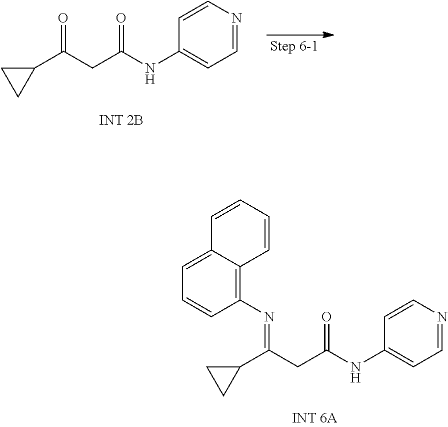 [SMILES: O=C(CC(=O)C1CC1)Nc1ccncc1 ; O=C(C/C(=N/c1cccc2ccccc12)C1CC1)Nc1ccncc1]

To a stirring solution of INT 2B (100 mg, 0.49 mmol) and naphthalen-1-amine (77 mg, 0.54 mmol) in CHCl₃ (7.5 mL) was added AcOH (32 mg, 0.54 mmol) portionwise at rt in a sealed tube. The tube was sealed, and the mixture was refluxed at 61° C. for 16 h. The reaction mixture was diluted with EA and washed with saturated aq. NaHCO₃ and brine, then dried (Na₂SO₄), filtered, concentrated, and purified by SiO₂ chromatography (MeOH/DCM) to provide 104 mg (approximately 16% pure, 10% yield) of 3-cyclopropyl-3-(naphthalen-1-ylimino)-N-(pyridin-4-yl) propanamide (INT 6A) as a brown oil that was used without further purification. LCMS-ESI (m/z) calculated for C₂₂H₁₉N₃O: 329.2, found 330.1 [M+H]⁺, t_{R}=6.29 min (Method 2).

### Step 6-2. Synthesis of 5-cyclopropyl-1-(naphthalen-1-yl)-N-(pyridin-4-yl)-1H-1,2,3-triazole-4-carboxamide (Compound 6-2)

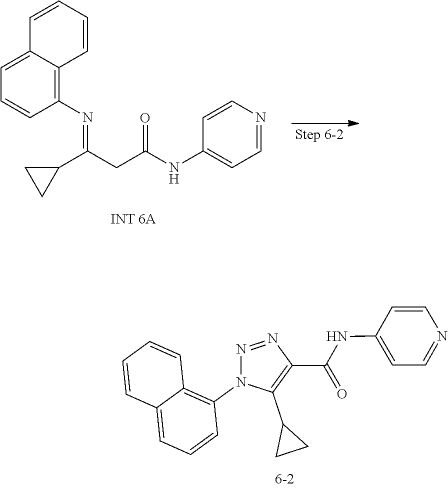 [SMILES: O=C(C/C(=N/c1cccc2ccccc12)C1CC1)Nc1ccncc1 ; O=C(Nc1ccncc1)c1nnn(-c2cccc3ccccc23)c1C1CC1]

To a stirred solution of INT 6A (approximately 17 mg, 0.05 mmol) in CH₃CN (5 mL) were added DBU (48 mg, 0.32 mmol) and a solution of N₃Ts (62 mg, 0.32 mmol 0.3 M in toluene) at 0° C. The reaction mixture was stirred at rt for 12 h and then heated to reflux at for 1 h. The reaction mixture was diluted with DMSO and then concentrated to remove CH₃CN and purified by reverse phase chromatography (MeOH/H₂O) to provide 20 mg (99%) of 5-cyclopropyl-1-(naphthalen-1-yl)-N-(pyridin-4-yl)-1H-1,2,3-triazole-4-carboxamide (Compound 6-2) as a white solid. LCMS-ESI (m/z) calculated for C₂₁H₁₇N₅O: 355.1; found 355.9 [M+H]⁺, t_{R}=11.02 min (Method 2). ¹H NMR (400 MHz, CDCl₃) δ 9.60 (s, 1H), 8.60 (s, 2H), 8.15 (d, J=8.0 Hz, 1H), 8.04 (d, J=8.0 Hz, 1H), 7.81 (d, J=4.0 Hz, 2H), 7.57-7.71 (m, 4H), 7.25 (d, J=8.0 Hz, 1H), 1.81-1.87 (m, 1H), 1.24 (d, J=8.0 Hz, 2H), 0.89 (d, J=8.0 Hz, 2H).

The compounds listed in Table 6 were made using the procedures of Scheme 6.

TABLE 6

| | | Purity | | | | |
|---|---|---|---|---|---|---|
| | Cpd | RT | | Observed | | Purity |
| Structure | No. | (min) | MW | m/z | Ion | Method |
| 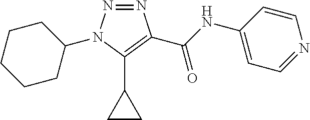 [SMILES: O=C(Nc1ccncc1)c1nnn(C2CCCCC2)c1C1CC1] | 6-1 | 10.85 | 311.4 | 312.2 | [M + H]⁺ | 2 |
| 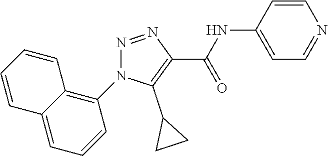 [SMILES: O=C(Nc1ccncc1)c1nnn(-c2cccc3ccccc23)c1C1CC1] | 6-2 | 11.06 | 355.4 | 356 | [M + H]⁺ | 2 |
| 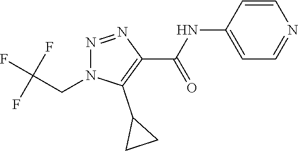 [SMILES: O=C(Nc1ccncc1)c1nnn(CC(F)(F)F)c1C1CC1] | 6-3 | 8.77 | 311.3 | 311.7 | [M + H]⁺ | 2 |
| 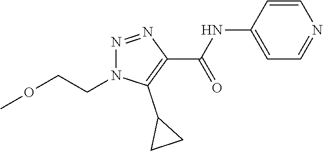 [SMILES: COCCn1nnc(C(=O)Nc2ccncc2)c1C1CC1] | 6-4 | 7.68 | 287.3 | 288.1 | [M + H]⁺ | 2 |
| 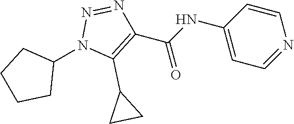 [SMILES: O=C(Nc1ccncc1)c1nnn(C2CCCC2)c1C1CC1] | 6-5 | 9.97 | 297.4 | 298.3 | [M + H]⁺ | 2 |

### Example 7

### Synthesis of Compound 7-12 and Other Representative Compounds

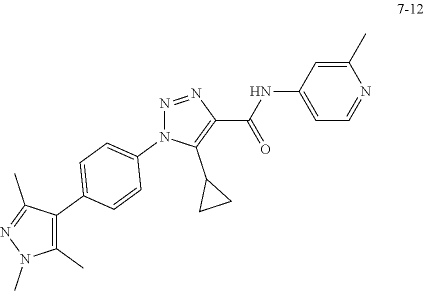 [SMILES: Cc1cc(NC(=O)c2nnn(-c3ccc(-c4c(C)nn(C)c4C)cc3)c2C2CC2)ccn1]

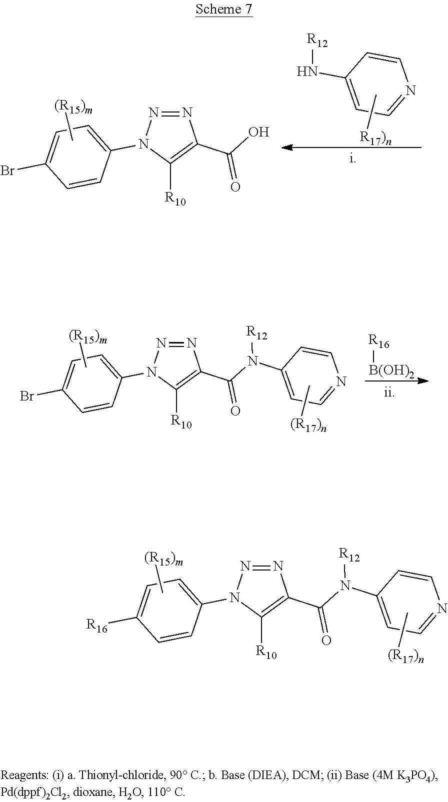

### Step 7-1. Synthesis of 1-(4-bromophenyl)-5-cyclopropyl-N-(2-methylpyridin-4-yl)-1H-1,2,3-triazole-4-carboxamide (INT 7A)

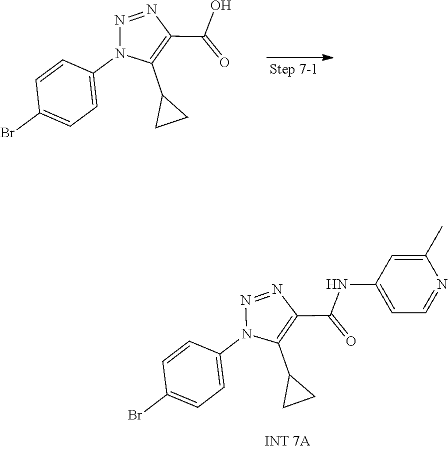 [SMILES: O=C(O)c1nnn(-c2ccc(Br)cc2)c1C1CC1 ; Cc1cc(NC(=O)c2nnn(-c3ccc(Br)cc3)c2C2CC2)ccn1]

A solution of 1-(4-bromophenyl)-5-cyclopropyl-1H-1,2,3-triazole-4-carboxylic acid (250 mg, 0.81 mmol) in thionyl chloride (118 μL, 0.9 mmol) was heated at 90° C. for 30 min. The mixture was concentrated, and the resulting residue was dissolved in DCM (20 mL). 2-methylpyridin-4-amine (96 mg, 0.9 mmol) and DIEA (314 mg, 2.4 mmol) were added. After stirring for 2 h, the reaction mixture was diluted with H₂O (10 mL), extracted with DCM, dried (Na₂SO₄), filtered, concentrated, and purified by SiO₂ chromatography (EA/hexanes) to afford 280 mg (87%) of 1-(4-bromophenyl)-5-cyclopropyl-N-(2-methylpyridin-4-yl)-1H-1,2,3-triazole-4-carboxamide (INT 7A). LCMS-ESI (m/z) calculated for C₁₈H₁₆BrN₅O: 427.2; found 428 [M+H]⁺, t_{R}=5.7 min (Method 3)

### Step 7-2. Synthesis of 5-cyclopropyl-N-(2-methylpyridin-4-yl)-1-(4-(1,3,5-tri methyl-1H-pyrazol-4-yl)phenyl)-1H-1,2,3-triazole-4-carboxamide (Compound 7-12)

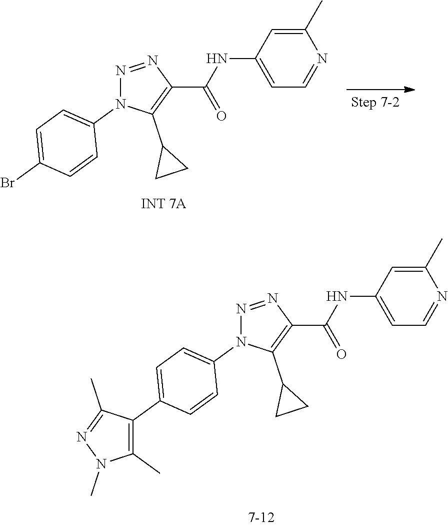 [SMILES: Cc1cc(NC(=O)c2nnn(-c3ccc(Br)cc3)c2C2CC2)ccn1 ; Cc1cc(NC(=O)c2nnn(-c3ccc(-c4c(C)nn(C)c4C)cc3)c2C2CC2)ccn1]

To a stirring solution (in a sealable tube) of INT 7A (60 mg, 0.15 μmol) in dioxane (5 mL) and H₂O (0.5 mL) were added 4M K₃PO₄ (96 mg, 0.45 mmol), 1,3,5-trimethyl-4-(4,4,5,5-tetramethyl-1,3,2-dioxaborolan-2-yl)-1H-pyrazole (71.1 mg, 0.3 mmol), Pd(dppf)₂Cl₂ (11 mg, 0.02 mmol). The tube was sealed, and the reaction mixture was stirred at 110° C. for 1 h. The tube was cooled, opened and additional 1,3,5-trimethyl-4-(4,4,5,5-tetramethyl-1,3,2-dioxaborolan-2-yl)-1H-pyrazole (35 mg, 0.15 mmol) and Pd(dppf)₂Cl₂ (11 mg, 0.02 mmol) were added. After heating at 110° C. for an additional 45 min, the reaction mixture was purified by SiO₂ chromatography (EA/hexane and MeOH/DCM). The fractions containing the product were combined and concentrated and the resulting solid was triturated with DCM/hexane, to provide 42 mg (65%) of 5-cyclopropyl-N-(2-methylpyridin-4-yl)-1-(4-(1,3,5-trimethyl-1H-pyrazol-4-yl)phenyl)-1H-1,2,3-triazole-4-carboxamidemethanamine (Compound 7-12) as a dark brown solid. LCMS-ESI (m/z) calculated for C₂₄H₂₅N₇O: 427.2: found 428.0 [M+H]⁺, t_{R}=10.86 min (Method 2) ¹H NMR (500 Hz, DMSO-D₆) 10.76 (s, 1H), 8.34 (d, J=8.0 Hz, 1H), 7.80 (s, 1H), 7.74 (d, J=8.0 Hz, 2H), 7.65 (d, J=4.0 Hz, 1H), 7.53 (d, J=8.0 Hz, 2H), 3.74 (s, 3H), 2.45 (s, 3H), 2.30 (s, 3H), 2.21 (s, 3H), 2.13 (t, J=8.0 Hz, 1H), 0.97 (d, J=8.0 Hz, 2H), 0.92 (t, J=4.0 Hz, 2H).

The compounds listed in the Table 7 were made using the procedures of Scheme 7.

TABLE 7

| | | Purity | | | | |
|---|---|---|---|---|---|---|
| | Cpd | RT | | Observed | | Purity |
| Structure | No. | (min) | MW | m/z | Ion | Method |
| 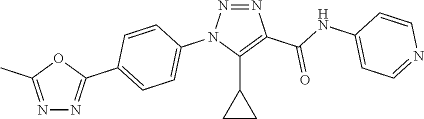 [SMILES: Cc1nnc(-c2ccc(-n3nnc(C(=O)Nc4ccncc4)c3C3CC3)cc2)o1] | 7-1 | 9.57 | 387.4 | 388.2 | [M + H]⁺ | 2 |
| 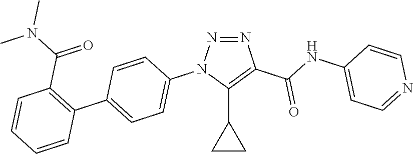 [SMILES: CN(C)C(=O)c1ccccc1-c1ccc(-n2nnc(C(=O)Nc3ccncc3)c2C2CC2)cc1] | 7-2 | 10.41 | 452.5 | 452.9 | [M + H]⁺ | 2 |
| 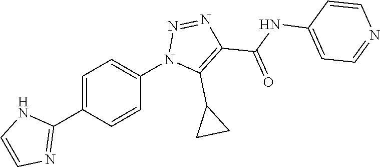 [SMILES: O=C(Nc1ccncc1)c1nnn(-c2ccc(-c3ncc[nH]3)cc2)c1C1CC1] | 7-3 | 7.57 | 371.4 | 371.9 | [M + H]⁺ | 2 |
| 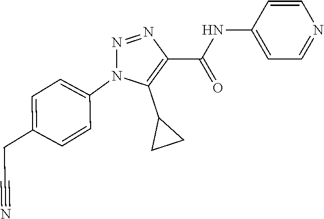 [SMILES: N#CCc1ccc(-n2nnc(C(=O)Nc3ccncc3)c2C2CC2)cc1] | 7-4 | 8.78 | 344.4 | 345.5 | [M + H]⁺ | 2 |
| 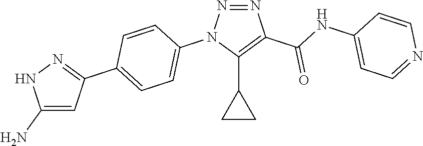 [SMILES: Nc1cc(-c2ccc(-n3nnc(C(=O)Nc4ccncc4)c3C3CC3)cc2)n[nH]1] | 7-5 | 8.09 | 386.4 | 386.9 | [M + H]⁺ | 2 |
| 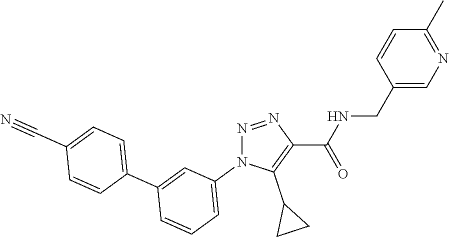 [SMILES: Cc1ccc(CNC(=O)c2nnn(-c3cccc(-c4ccc(C#N)cc4)c3)c2C2CC2)cn1] | 7-7 | 10.47 | 434.5 | 435.1 | [M + H]⁺ | 2 |
| 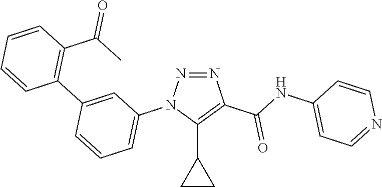 [SMILES: CC(=O)c1ccccc1-c1cccc(-n2nnc(C(=O)Nc3ccncc3)c2C2CC2)c1] | 7-8 | 12.26 | 423.5 | 423.8 | [M + H]⁺ | 2 |
| 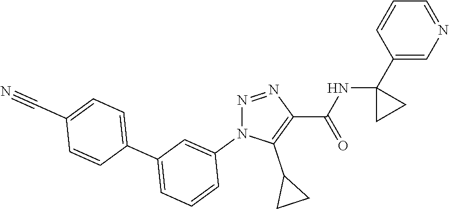 [SMILES: N#Cc1ccc(-c2cccc(-n3nnc(C(=O)NC4(c5cccnc5)CC4)c3C3CC3)c2)cc1] | 7-9 | 10.73 | 446.5 | 446.8 | [M + H]⁺ | 2 |
| 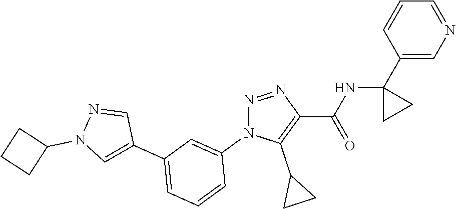 [SMILES: O=C(NC1(c2cccnc2)CC1)c1nnn(-c2cccc(-c3cnn(C4CCC4)c3)c2)c1C1CC1] | 7-10 | 10.84 | 465.6 | 465.7 | [M + H]⁺ | 2 |
| 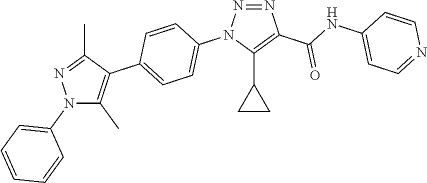 [SMILES: Cc1nn(-c2ccccc2)c(C)c1-c1ccc(-n2nnc(C(=O)Nc3ccncc3)c2C2CC2)cc1] | 7-11 | 11.9 | 475.6 | 476.1 | [M + H]⁺ | 2 |
| 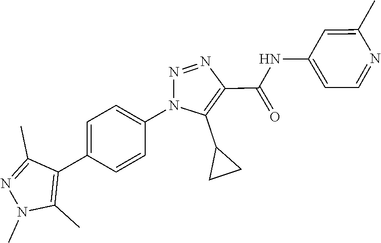 [SMILES: Cc1cc(NC(=O)c2nnn(-c3ccc(-c4c(C)nn(C)c4C)cc3)c2C2CC2)ccn1] | 7-12 | 10.86 | 427.5 | 428.3 | [M + H]⁺ | 2 |
| 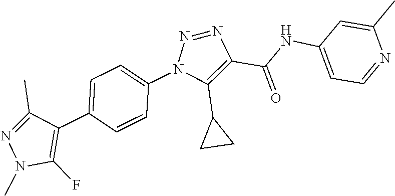 | 7-13 | 11.26 | 431.5 | 431.9 | [M + H]⁺ | 2 |
| 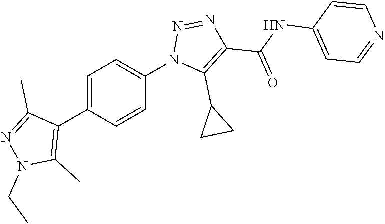 [SMILES: CCn1nc(C)c(-c2ccc(-n3nnc(C(=O)Nc4ccncc4)c3C3CC3)cc2)c1C] | 7-14 | 10.82 | 427.5 | 428.3 | [M + H]⁺ | 2 |
| 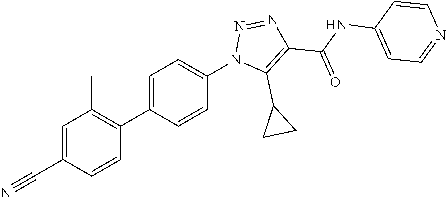 [SMILES: Cc1cc(C#N)ccc1-c1ccc(-n2nnc(C(=O)Nc3ccncc3)c2C2CC2)cc1] | 7-15 | 11.61 | 420.5 | 420.7 | [M + H]⁺ | 2 |
| 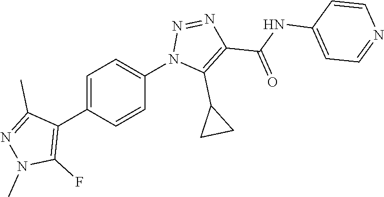 [SMILES: Cc1nn(C)c(F)c1-c1ccc(-n2nnc(C(=O)Nc3ccncc3)c2C2CC2)cc1] | 7-16 | 10.94 | 417.4 | 417.9 | [M + H]⁺ | 2 |
| 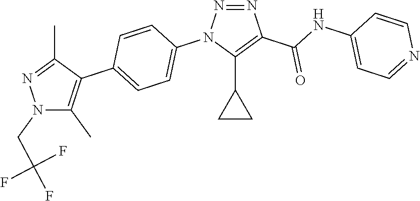 [SMILES: Cc1nn(CC(F)(F)F)c(C)c1-c1ccc(-n2nnc(C(=O)Nc3ccncc3)c2C2CC2)cc1] | 7-17 | 11.06 | 481.5 | 482.2 | [M + H]⁺ | 2 |
| 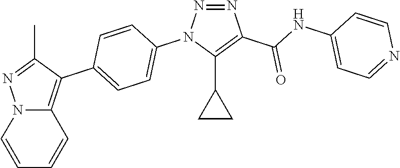 [SMILES: Cc1nn2ccccc2c1-c1ccc(-n2nnc(C(=O)Nc3ccncc3)c2C2CC2)cc1] | 7-18 | 11.41 | 435.5 | 436.1 | [M + H]⁺ | 2 |
| 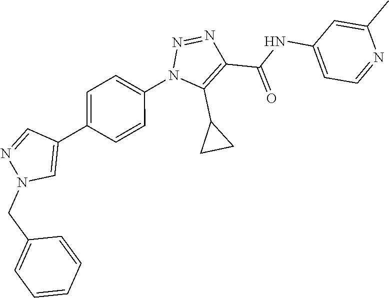 [SMILES: Cc1cc(NC(=O)c2nnn(-c3ccc(-c4cnn(Cc5ccccc5)c4)cc3)c2C2CC2)ccn1] | 7-19 | 11.64 | 475.6 | 475.9 | [M + H]⁺ | 2 |
| 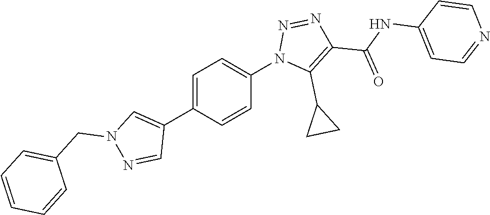 [SMILES: O=C(Nc1ccncc1)c1nnn(-c2ccc(-c3cnn(Cc4ccccc4)c3)cc2)c1C1CC1] | 7-20 | 11.64 | 461.5 | 461.8 | [M + H]⁺ | 2 |
| 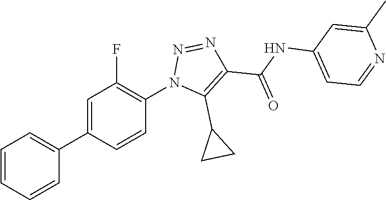 [SMILES: Cc1cc(NC(=O)c2nnn(-c3ccc(-c4ccccc4)cc3F)c2C2CC2)ccn1] | 7-21 | 11.75 | 413.5 | 414.3 | [M + H]⁺ | 2 |
| 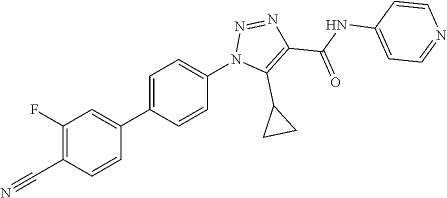 [SMILES: N#Cc1ccc(-c2ccc(-n3nnc(C(=O)Nc4ccncc4)c3C3CC3)cc2)cc1F] | 7-22 | 11.45 | 424.4 | 424.9 | [M + H]⁺ | 2 |
| 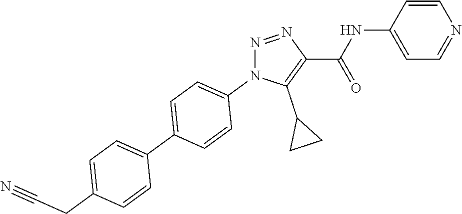 [SMILES: N#CCc1ccc(-c2ccc(-n3nnc(C(=O)Nc4ccncc4)c3C3CC3)cc2)cc1] | 7-23 | 11.09 | 420.5 | 421 | [M + H]⁺ | 2 |
| 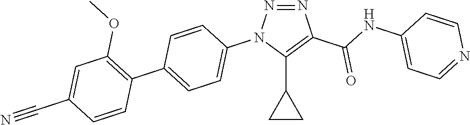 [SMILES: COc1cc(C#N)ccc1-c1ccc(-n2nnc(C(=O)Nc3ccncc3)c2C2CC2)cc1] | 7-24 | 11.52 | 436.5 | 437 | [M + H]⁺ | 2 |
| 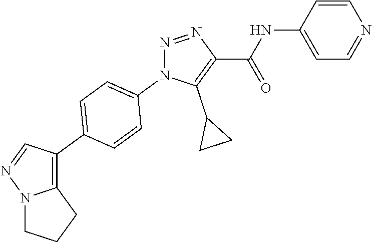 [SMILES: O=C(Nc1ccncc1)c1nnn(-c2ccc(-c3cnn4c3CCC4)cc2)c1C1CC1] | 7-25 | 10.72 | 411.5 | 412 | [M + H]⁺ | 2 |
| 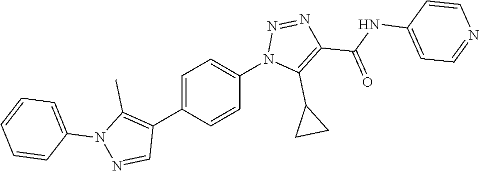 [SMILES: Cc1c(-c2ccc(-n3nnc(C(=O)Nc4ccncc4)c3C3CC3)cc2)cnn1-c1ccccc1] | 7-26 | 11.61 | 461.5 | 462 | [M + H]⁺ | 2 |
| 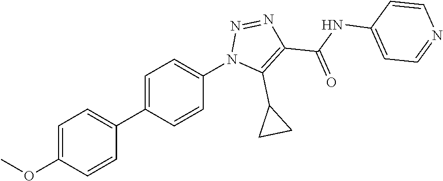 [SMILES: COc1ccc(-c2ccc(-n3nnc(C(=O)Nc4ccncc4)c3C3CC3)cc2)cc1] | 7-27 | 12.2 | 411.5 | 411.9 | [M + H]⁺ | 2 |
| 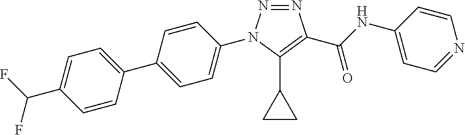 [SMILES: O=C(Nc1ccncc1)c1nnn(-c2ccc(-c3ccc(C(F)F)cc3)cc2)c1C1CC1] | 7-28 | 11.65 | 431.4 | 432.1 | [M + H]⁺ | 2 |
| 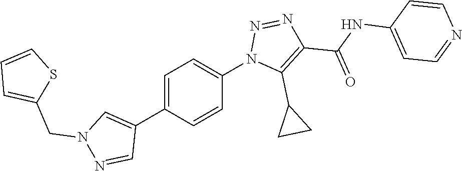 [SMILES: O=C(Nc1ccncc1)c1nnn(-c2ccc(-c3cnn(Cc4cccs4)c3)cc2)c1C1CC1] | 7-29 | 11.12 | 467.6 | 468.2 | [M + H]⁺ | 2 |
| 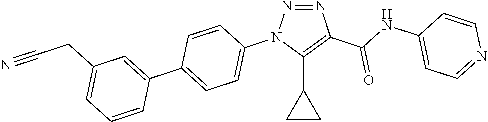 [SMILES: N#CCc1cccc(-c2ccc(-n3nnc(C(=O)Nc4ccncc4)c3C3CC3)cc2)c1] | 7-30 | 11.15 | 420.5 | 421 | [M + H]⁺ | 2 |
| 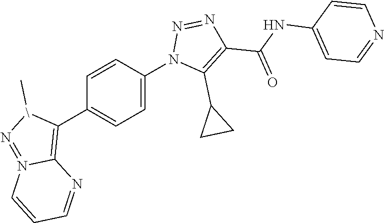 | 7-31 | 10.94 | 436.5 | 436.9 | [M + H]⁺ | 2 |
| 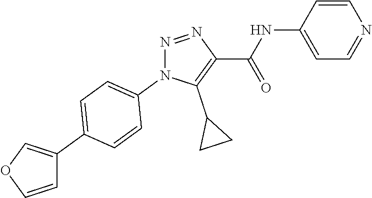 [SMILES: O=C(Nc1ccncc1)c1nnn(-c2ccc(-c3ccoc3)cc2)c1C1CC1] | 7-32 | 11.43 | 371.4 | 372 | [M + H]⁺ | 2 |
| 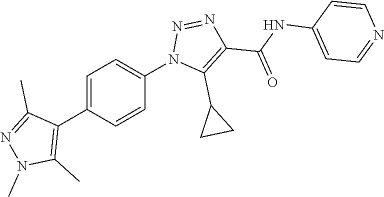 [SMILES: Cc1nn(C)c(C)c1-c1ccc(-n2nnc(C(=O)Nc3ccncc3)c2C2CC2)cc1] | 7-33 | 10.54 | 413.5 | 414.3 | [M + H]⁺ | 2 |
| 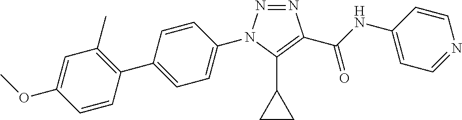 [SMILES: COc1ccc(-c2ccc(-n3nnc(C(=O)Nc4ccncc4)c3C3CC3)cc2)c(C)c1] | 7-34 | 12.81 | 425.5 | 426.1 | [M + H]⁺ | 2 |
| 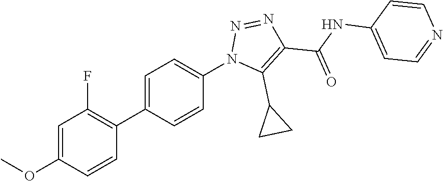 [SMILES: COc1ccc(-c2ccc(-n3nnc(C(=O)Nc4ccncc4)c3C3CC3)cc2)c(F)c1] | 7-35 | 12.43 | 429.5 | 430.3 | [M + H]⁺ | 2 |
| 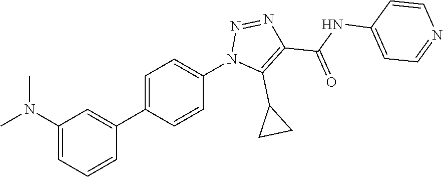 [SMILES: CN(C)c1cccc(-c2ccc(-n3nnc(C(=O)Nc4ccncc4)c3C3CC3)cc2)c1] | 7-36 | 9.9 | 424.5 | 424.9 | [M + H]⁺ | 2 |
| 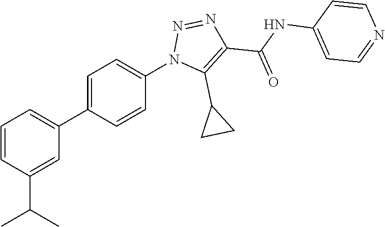 [SMILES: CC(C)c1cccc(-c2ccc(-n3nnc(C(=O)Nc4ccncc4)c3C3CC3)cc2)c1] | 7-37 | 13.53 | 423.5 | 424 | [M + H]⁺ | 2 |
| 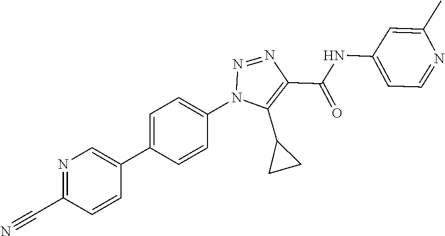 [SMILES: Cc1cc(NC(=O)c2nnn(-c3ccc(-c4ccc(C#N)nc4)cc3)c2C2CC2)ccn1] | 7-38 | 10.25 | 421.5 | 421.9 | [M + H]⁺ | 2 |
| 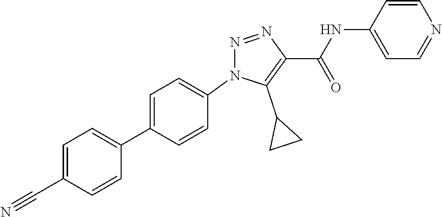 [SMILES: N#Cc1ccc(-c2ccc(-n3nnc(C(=O)Nc4ccncc4)c3C3CC3)cc2)cc1] | 7-39 | 11.26 | 406.4 | 406.8 | [M + H]⁺ | 2 |
| 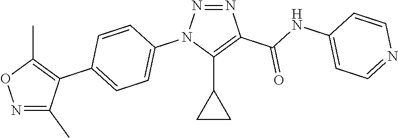 [SMILES: Cc1noc(C)c1-c1ccc(-n2nnc(C(=O)Nc3ccncc3)c2C2CC2)cc1] | 7-40 | 10.59 | 400.4 | 401 | [M + H]⁺ | 2 |
| 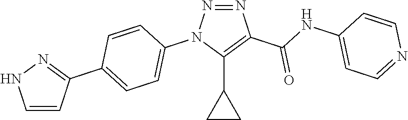 [SMILES: O=C(Nc1ccncc1)c1nnn(-c2ccc(-c3cc[nH]n3)cc2)c1C1CC1] | 7-41 | 10 | 371.4 | 372 | [M + H]⁺ | 2 |
| 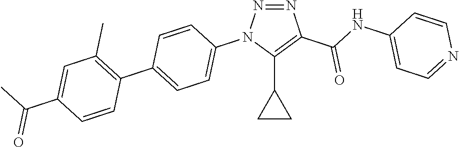 [SMILES: CC(=O)c1ccc(-c2ccc(-n3nnc(C(=O)Nc4ccncc4)c3C3CC3)cc2)c(C)c1] | 7-42 | 11.7 | 437.5 | 438.2 | [M + H]⁺ | 2 |
| 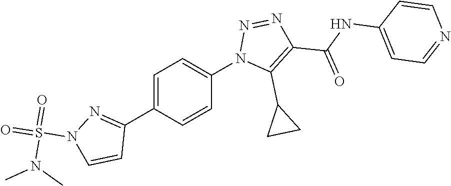 [SMILES: CN(C)S(=O)(=O)n1ccc(-c2ccc(-n3nnc(C(=O)Nc4ccncc4)c3C3CC3)cc2)n1] | 7-43 | 10.77 | 478.5 | 478.8 | [M + H]⁺ | 2 |
| 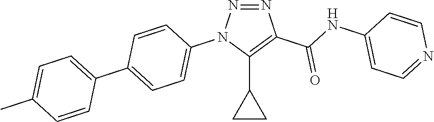 [SMILES: Cc1ccc(-c2ccc(-n3nnc(C(=O)Nc4ccncc4)c3C3CC3)cc2)cc1] | 7-44 | 13.03 | 395.5 | 395.5 | [M + H]⁺ | 2 |
| 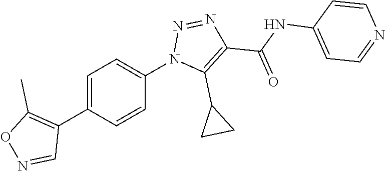 [SMILES: Cc1oncc1-c1ccc(-n2nnc(C(=O)Nc3ccncc3)c2C2CC2)cc1] | 7-45 | 10.34 | 386.4 | 386.9 | [M + H]⁺ | 2 |
| 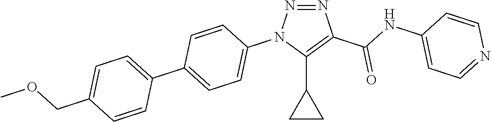 [SMILES: COCc1ccc(-c2ccc(-n3nnc(C(=O)Nc4ccncc4)c3C3CC3)cc2)cc1] | 7-46 | 12.12 | 425.5 | 426.1 | [M + H]⁺ | 2 |
| 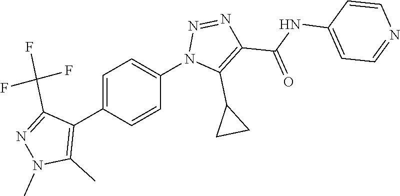 [SMILES: Cc1c(-c2ccc(-n3nnc(C(=O)Nc4ccncc4)c3C3CC3)cc2)c(C(F)(F)F)nn1C] | 7-47 | 11.16 | 467.5 | 468 | [M + H]⁺ | 2 |
| 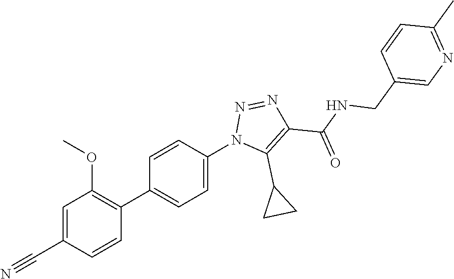 [SMILES: COc1cc(C#N)ccc1-c1ccc(-n2nnc(C(=O)NCc3ccc(C)nc3)c2C2CC2)cc1] | 7-48 | 10.76 | 464.5 | 464.8 | [M + H]⁺ | 2 |
| 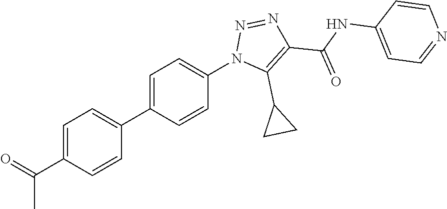 [SMILES: CC(=O)c1ccc(-c2ccc(-n3nnc(C(=O)Nc4ccncc4)c3C3CC3)cc2)cc1] | 7-49 | 11.41 | 423.5 | 423.8 | [M + H]⁺ | 2 |
| 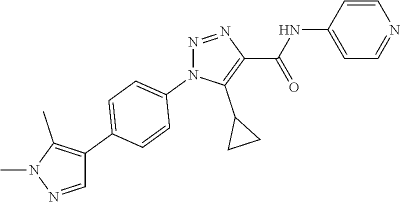 [SMILES: Cc1c(-c2ccc(-n3nnc(C(=O)Nc4ccncc4)c3C3CC3)cc2)cnn1C] | 7-50 | 10.47 | 399.5 | 400.3 | [M + H]⁺ | 2 |
| 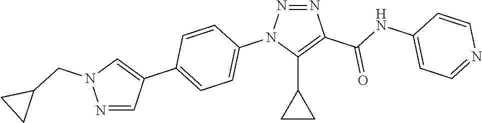 [SMILES: O=C(Nc1ccncc1)c1nnn(-c2ccc(-c3cnn(CC4CC4)c3)cc2)c1C1CC1] | 7-51 | 11.19 | 425.5 | 426.1 | [M + H]⁺ | 2 |
| 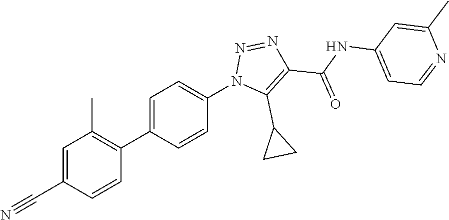 [SMILES: Cc1cc(NC(=O)c2nnn(-c3ccc(-c4ccc(C#N)cc4C)cc3)c2C2CC2)ccn1] | 7-52 | 11.81 | 434.5 | 434.8 | [M + H]⁺ | 2 |
| 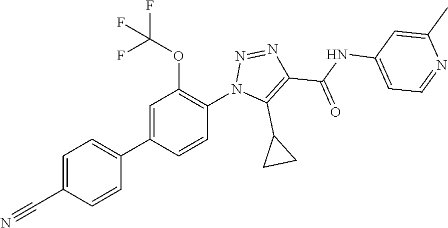 [SMILES: Cc1cc(NC(=O)c2nnn(-c3ccc(-c4ccc(C#N)cc4)cc3OC(F)(F)F)c2C2CC2)ccn1] | 7-53 | 11.47 | 504.5 | 504.7 | [M + H]⁺ | 2 |
| 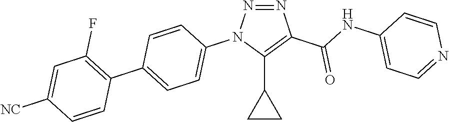 [SMILES: N#Cc1ccc(-c2ccc(-n3nnc(C(=O)Nc4ccncc4)c3C3CC3)cc2)c(F)c1] | 7-54 | 11.49 | 424.4 | 424.9 | [M + H]⁺ | 2 |
| 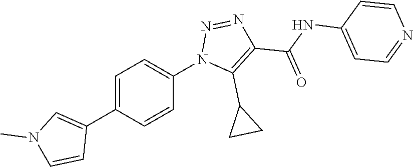 [SMILES: Cn1ccc(-c2ccc(-n3nnc(C(=O)Nc4ccncc4)c3C3CC3)cc2)c1] | 7-55 | 11.49 | 384.4 | 385.1 | [M + H]⁺ | 2 |
| 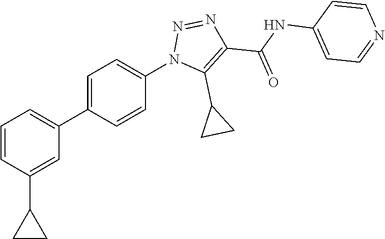 [SMILES: O=C(Nc1ccncc1)c1nnn(-c2ccc(-c3cccc(C4CC4)c3)cc2)c1C1CC1] | 7-56 | 13.19 | 421.5 | 422 | [M + H]⁺ | 2 |
| 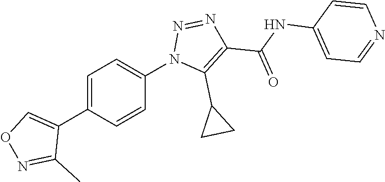 [SMILES: Cc1nocc1-c1ccc(-n2nnc(C(=O)Nc3ccncc3)c2C2CC2)cc1] | 7-57 | 10.2 | 386.4 | 387 | [M + H]⁺ | 2 |
| 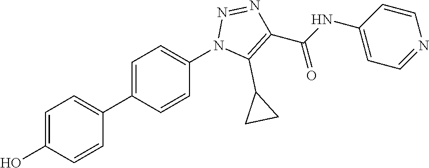 [SMILES: O=C(Nc1ccncc1)c1nnn(-c2ccc(-c3ccc(O)cc3)cc2)c1C1CC1] | 7-58 | 10.84 | 397.4 | 397.8 | [M + H]⁺ | 2 |
| 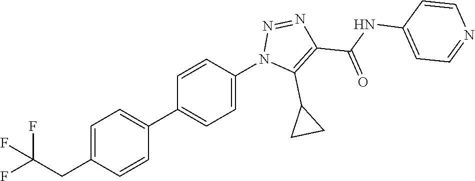 [SMILES: O=C(Nc1ccncc1)c1nnn(-c2ccc(-c3ccc(CC(F)(F)F)cc3)cc2)c1C1CC1] | 7-59 | 12.31 | 463.5 | 463.8 | [M + H]⁺ | 2 |
| 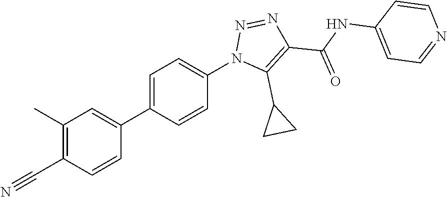 [SMILES: Cc1cc(-c2ccc(-n3nnc(C(=O)Nc4ccncc4)c3C3CC3)cc2)ccc1C#N] | 7-60 | 11.92 | 420.5 | 421 | [M + H]⁺ | 2 |
| 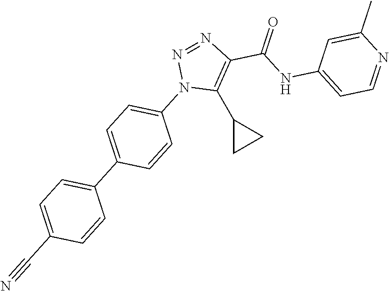 [SMILES: Cc1cc(NC(=O)c2nnn(-c3ccc(-c4ccc(C#N)cc4)cc3)c2C2CC2)ccn1] | 7-61 | 11.6 | 420.5 | 421 | [M + H]⁺ | 2 |
| 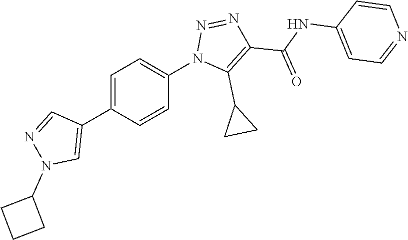 [SMILES: O=C(Nc1ccncc1)c1nnn(-c2ccc(-c3cnn(C4CCC4)c3)cc2)c1C1CC1] | 7-62 | 11.13 | 425.5 | 426.2 | [M + H]⁺ | 2 |
| 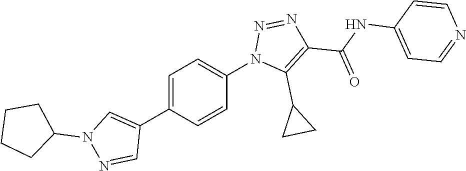 [SMILES: O=C(Nc1ccncc1)c1nnn(-c2ccc(-c3cnn(C4CCCC4)c3)cc2)c1C1CC1] | 7-63 | 11.63 | 439.5 | 440.4 | [M + H]⁺ | 2 |
| 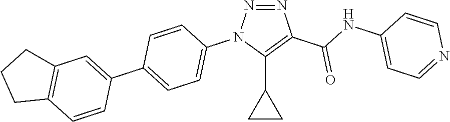 [SMILES: O=C(Nc1ccncc1)c1nnn(-c2ccc(-c3ccc4c(c3)CCC4)cc2)c1C1CC1] | 7-64 | 13.5 | 421.5 | 422.1 | [M + H]⁺ | 2 |
| 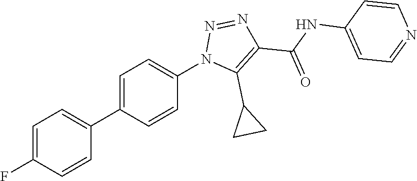 [SMILES: O=C(Nc1ccncc1)c1nnn(-c2ccc(-c3ccc(F)cc3)cc2)c1C1CC1] | 7-65 | 12.18 | 399.4 | 400 | [M + H]⁺ | 2 |
| 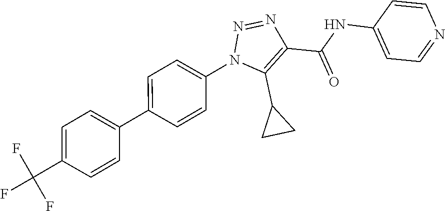 [SMILES: O=C(Nc1ccncc1)c1nnn(-c2ccc(-c3ccc(C(F)(F)F)cc3)cc2)c1C1CC1] | 7-66 | 12.76 | 449.4 | 450 | [M + H]⁺ | 2 |
| 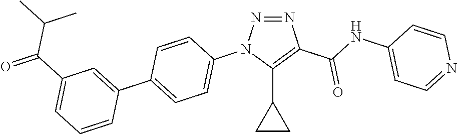 [SMILES: CC(C)C(=O)c1cccc(-c2ccc(-n3nnc(C(=O)Nc4ccncc4)c3C3CC3)cc2)c1] | 7-67 | 12.41 | 451.5 | 451.9 | [M + H]⁺ | 2 |
| 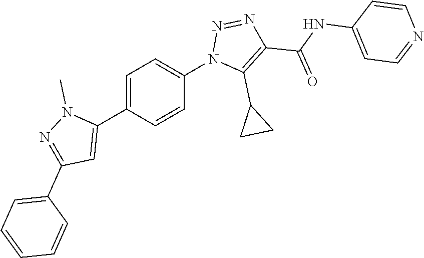 [SMILES: Cn1nc(-c2ccccc2)cc1-c1ccc(-n2nnc(C(=O)Nc3ccncc3)c2C2CC2)cc1] | 7-68 | 12.11 | 461.5 | 461.9 | [M + H]⁺ | 2 |
| 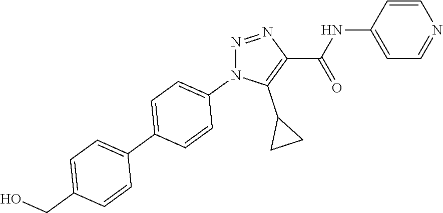 [SMILES: O=C(Nc1ccncc1)c1nnn(-c2ccc(-c3ccc(CO)cc3)cc2)c1C1CC1] | 7-69 | 10.87 | 411.5 | 411.9 | [M + H]⁺ | 2 |
| 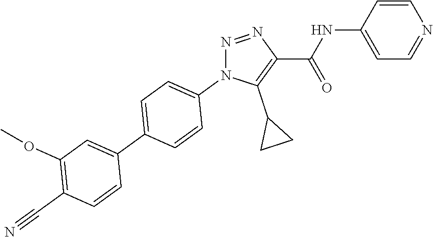 [SMILES: COc1cc(-c2ccc(-n3nnc(C(=O)Nc4ccncc4)c3C3CC3)cc2)ccc1C#N] | 7-70 | 11.2 | 436.5 | 437.1 | [M + H]⁺ | 2 |
| 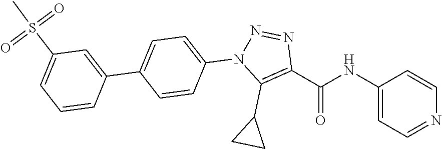 [SMILES: CS(=O)(=O)c1cccc(-c2ccc(-n3nnc(C(=O)Nc4ccncc4)c3C3CC3)cc2)c1] | 7-71 | 10.38 | 459.5 | 459.6 | [M + H]⁺ | 2 |
| 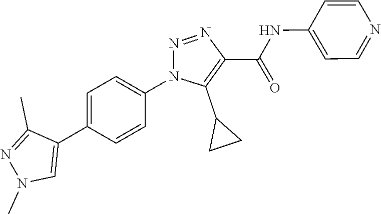 [SMILES: Cc1nn(C)cc1-c1ccc(-n2nnc(C(=O)Nc3ccncc3)c2C2CC2)cc1] | 7-72 | 10.4 | 399.5 | 400.2 | [M + H]⁺ | 2 |
| 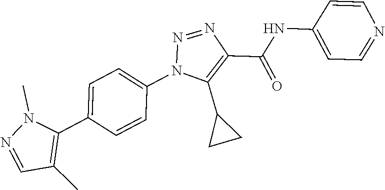 [SMILES: Cc1cnn(C)c1-c1ccc(-n2nnc(C(=O)Nc3ccncc3)c2C2CC2)cc1] | 7-73 | 10.49 | 399.5 | 400.2 | [M + H]⁺ | 2 |
| 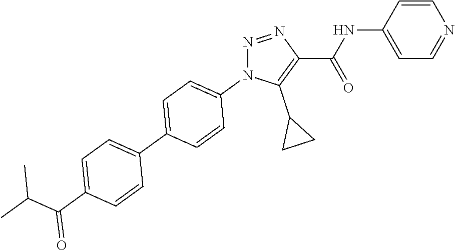 [SMILES: CC(C)C(=O)c1ccc(-c2ccc(-n3nnc(C(=O)Nc4ccncc4)c3C3CC3)cc2)cc1] | 7-74 | 12.46 | 451.5 | 451.6 | [M + H]⁺ | 2 |
| 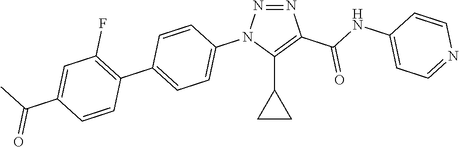 [SMILES: CC(=O)c1ccc(-c2ccc(-n3nnc(C(=O)Nc4ccncc4)c3C3CC3)cc2)c(F)c1] | 7-75 | 11.45 | 441.5 | 422.3 | [M + H]⁺ | 2 |
| 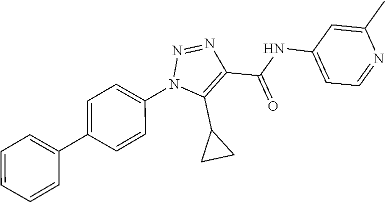 [SMILES: Cc1cc(NC(=O)c2nnn(-c3ccc(-c4ccccc4)cc3)c2C2CC2)ccn1] | 7-76 | 12.25 | 395.5 | 396.1 | [M + H]⁺ | 2 |
| 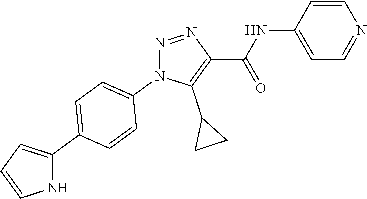 [SMILES: O=C(Nc1ccncc1)c1nnn(-c2ccc(-c3ccc[nH]3)cc2)c1C1CC1] | 7-77 | 11.26 | 370.4 | 371.1 | [M + H]⁺ | 2 |
| 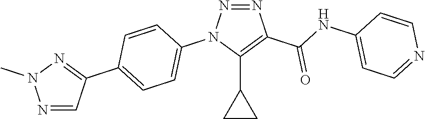 [SMILES: Cn1ncc(-c2ccc(-n3nnc(C(=O)Nc4ccncc4)c3C3CC3)cc2)n1] | 7-78 | 10.44 | 386.4 | 387 | [M + H]⁺ | 2 |
| 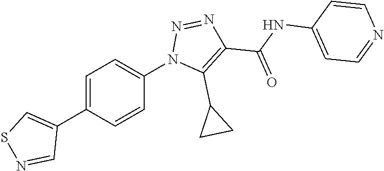 [SMILES: O=C(Nc1ccncc1)c1nnn(-c2ccc(-c3cnsc3)cc2)c1C1CC1] | 7-79 | 10.47 | 388.4 | 389.2 | [M + H]⁺ | 2 |
| 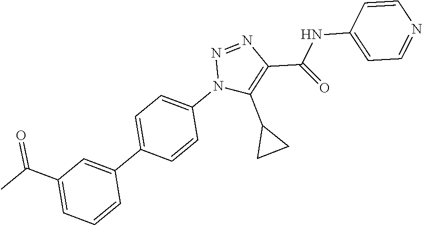 [SMILES: CC(=O)c1cccc(-c2ccc(-n3nnc(C(=O)Nc4ccncc4)c3C3CC3)cc2)c1] | 7-80 | 11.34 | 423.5 | 423.8 | [M + H]⁺ | 2 |
| 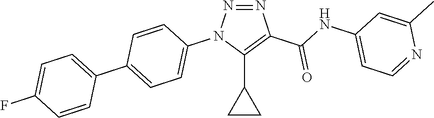 [SMILES: Cc1cc(NC(=O)c2nnn(-c3ccc(-c4ccc(F)cc4)cc3)c2C2CC2)ccn1] | 7-81 | 12.03 | 413.5 | 414.2 | [M + H]⁺ | 2 |
| 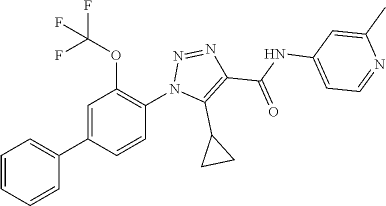 [SMILES: Cc1cc(NC(=O)c2nnn(-c3ccc(-c4ccccc4)cc3OC(F)(F)F)c2C2CC2)ccn1] | 7-82 | 12.42 | 479.5 | 479.9 | [M + H]⁺ | 2 |
| 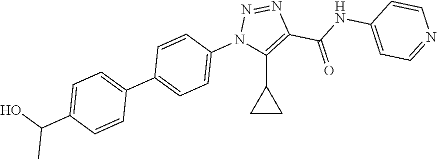 [SMILES: CC(O)c1ccc(-c2ccc(-n3nnc(C(=O)Nc4ccncc4)c3C3CC3)cc2)cc1] | 7-83 | 11.33 | 425.5 | 426.2 | [M + H]⁺ | 2 |
| 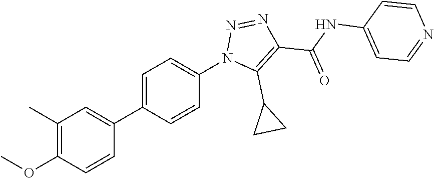 [SMILES: COc1ccc(-c2ccc(-n3nnc(C(=O)Nc4ccncc4)c3C3CC3)cc2)cc1C] | 7-84 | 12.91 | 425.5 | 425.8 | [M + H]⁺ | 2 |
| 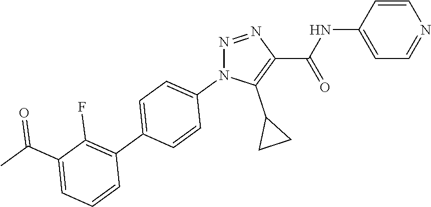 [SMILES: CC(=O)c1cccc(-c2ccc(-n3nnc(C(=O)Nc4ccncc4)c3C3CC3)cc2)c1F] | 7-85 | 11.49 | 441.5 | 442 | [M + H]⁺ | 2 |
| 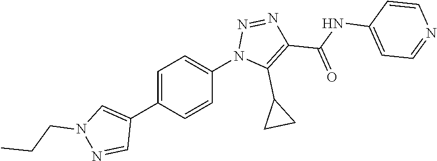 [SMILES: CCCn1cc(-c2ccc(-n3nnc(C(=O)Nc4ccncc4)c3C3CC3)cc2)cn1] | 7-86 | 10.99 | 413.5 | 414.1 | [M + H]⁺ | 2 |
| 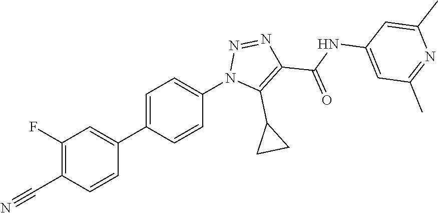 [SMILES: Cc1cc(NC(=O)c2nnn(-c3ccc(-c4ccc(C#N)c(F)c4)cc3)c2C2CC2)cc(C)n1] | 7-87 | 11.65 | 452.5 | 452.8 | [M + H]⁺ | 2 |
| 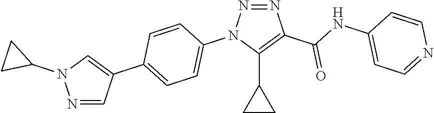 [SMILES: O=C(Nc1ccncc1)c1nnn(-c2ccc(-c3cnn(C4CC4)c3)cc2)c1C1CC1] | 7-88 | 10.83 | 411.5 | 412.1 | [M + H]⁺ | 2 |
| 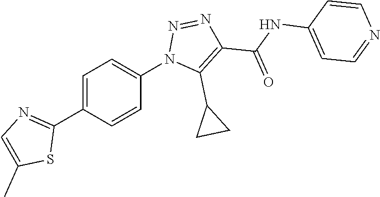 [SMILES: Cc1cnc(-c2ccc(-n3nnc(C(=O)Nc4ccncc4)c3C3CC3)cc2)s1] | 7-89 | 11.23 | 402.5 | 402.9 | [M + H]⁺ | 2 |
| 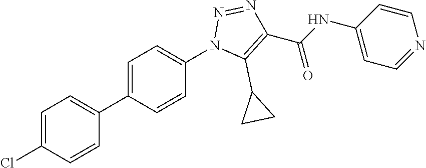 [SMILES: O=C(Nc1ccncc1)c1nnn(-c2ccc(-c3ccc(Cl)cc3)cc2)c1C1CC1] | 7-90 | 12.78 | 415.9 | 415.9 | [M + H]⁺ | 2 |
| 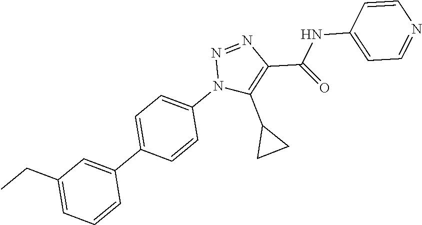 [SMILES: CCc1cccc(-c2ccc(-n3nnc(C(=O)Nc4ccncc4)c3C3CC3)cc2)c1] | 7-91 | 13.11 | 409.5 | 409.5 | [M + H]⁺ | 2 |
| 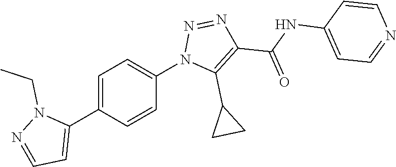 [SMILES: CCn1nccc1-c1ccc(-n2nnc(C(=O)Nc3ccncc3)c2C2CC2)cc1] | 7-92 | 10.49 | 399.5 | 400.1 | [M + H]⁺ | 2 |
| 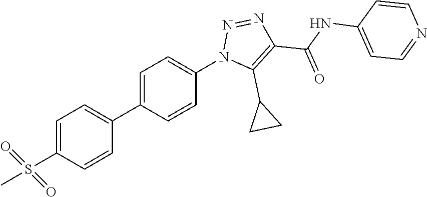 [SMILES: CS(=O)(=O)c1ccc(-c2ccc(-n3nnc(C(=O)Nc4ccncc4)c3C3CC3)cc2)cc1] | 7-93 | 10.26 | 459.5 | 460.1 | [M + H]⁺ | 2 |
| 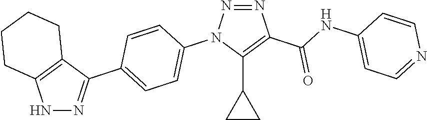 [SMILES: O=C(Nc1ccncc1)c1nnn(-c2ccc(-c3n[nH]c4c3CCCC4)cc2)c1C1CC1] | 7-94 | 11.18 | 425.5 | 425.8 | [M + H]⁺ | 2 |
| 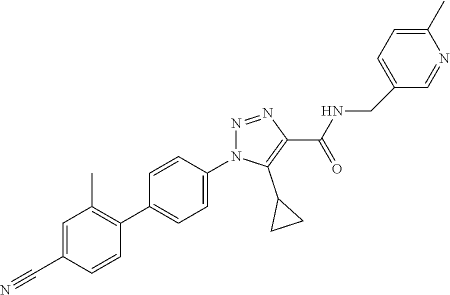 [SMILES: Cc1ccc(CNC(=O)c2nnn(-c3ccc(-c4ccc(C#N)cc4C)cc3)c2C2CC2)cn1] | 7-95 | 11.01 | 448.5 | 449 | [M + H]⁺ | 2 |
| 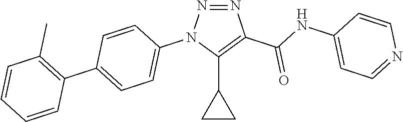 [SMILES: Cc1ccccc1-c1ccc(-n2nnc(C(=O)Nc3ccncc3)c2C2CC2)cc1] | 7-96 | 12.44 | 395.5 | 395.8 | [M + H]⁺ | 2 |
| 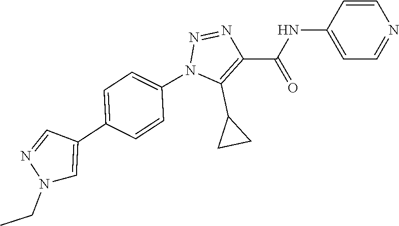 [SMILES: CCn1cc(-c2ccc(-n3nnc(C(=O)Nc4ccncc4)c3C3CC3)cc2)cn1] | 7-97 | 10.59 | 399.5 | 400.2 | [M + H]⁺ | 2 |
| 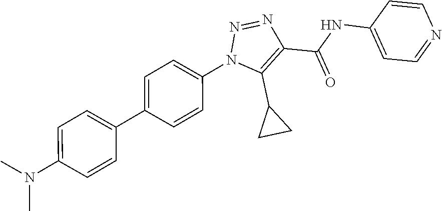 [SMILES: CN(C)c1ccc(-c2ccc(-n3nnc(C(=O)Nc4ccncc4)c3C3CC3)cc2)cc1] | 7-98 | 10.41 | 424.5 | 424.9 | [M + H]⁺ | 2 |
| 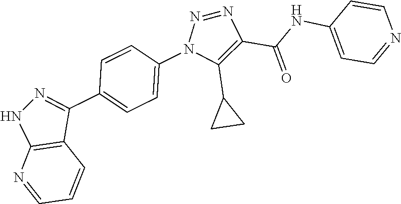 [SMILES: O=C(Nc1ccncc1)c1nnn(-c2ccc(-c3n[nH]c4ncccc34)cc2)c1C1CC1] | 7-99 | 10.64 | 422.5 | 422.8 | [M + H]⁺ | 2 |
| 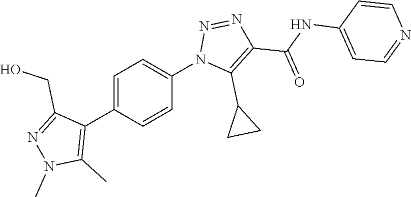 [SMILES: Cc1c(-c2ccc(-n3nnc(C(=O)Nc4ccncc4)c3C3CC3)cc2)c(CO)nn1C] | 7-100 | 9.76 | 429.5 | 430.2 | [M + H]⁺ | 2 |
| 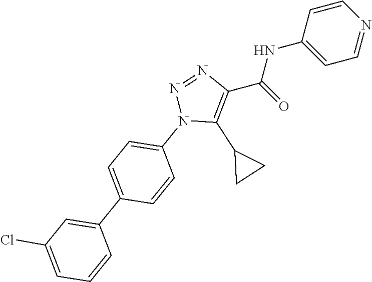 [SMILES: O=C(Nc1ccncc1)c1nnn(-c2ccc(-c3cccc(Cl)c3)cc2)c1C1CC1] | 7-101 | 12.85 | 415.9 | 416.1 | [M + H]⁺ | 2 |
| 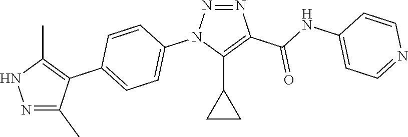 [SMILES: Cc1n[nH]c(C)c1-c1ccc(-n2nnc(C(=O)Nc3ccncc3)c2C2CC2)cc1] | 7-102 | 9.73 | 399.5 | 400.1 | [M + H]⁺ | 2 |
| 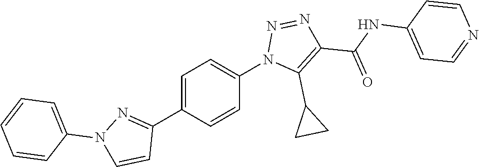 [SMILES: O=C(Nc1ccncc1)c1nnn(-c2ccc(-c3ccn(-c4ccccc4)n3)cc2)c1C1CC1] | 7-103 | 12.33 | 447.5 | 447.5 | [M + H]⁺ | 2 |
| 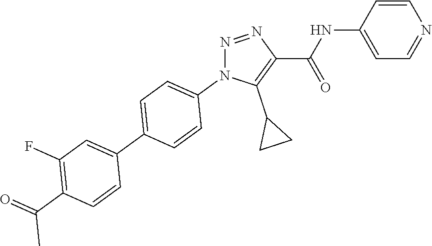 [SMILES: CC(=O)c1ccc(-c2ccc(-n3nnc(C(=O)Nc4ccncc4)c3C3CC3)cc2)cc1F] | 7-104 | 11.73 | 441.5 | 442.4 | [M + H]⁺ | 2 |
| 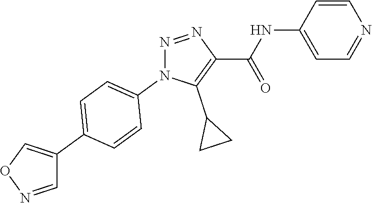 [SMILES: O=C(Nc1ccncc1)c1nnn(-c2ccc(-c3cnoc3)cc2)c1C1CC1] | 7-105 | 9.77 | 372.4 | 373 | [M + H]⁺ | 2 |
| 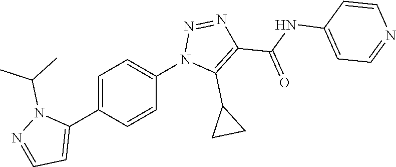 [SMILES: CC(C)n1nccc1-c1ccc(-n2nnc(C(=O)Nc3ccncc3)c2C2CC2)cc1] | 7-106 | 10.82 | 413.5 | 413.7 | [M + H]⁺ | 2 |
| 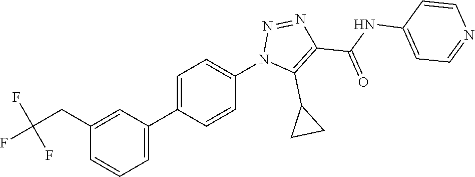 [SMILES: O=C(Nc1ccncc1)c1nnn(-c2ccc(-c3cccc(CC(F)(F)F)c3)cc2)c1C1CC1] | 7-107 | 12.41 | 463.5 | 464 | [M + H]⁺ | 2 |
| 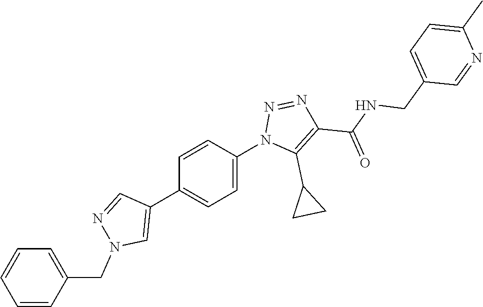 [SMILES: Cc1ccc(CNC(=O)c2nnn(-c3ccc(-c4cnn(Cc5ccccc5)c4)cc3)c2C2CC2)cn1] | 7-108 | 10.91 | 489.6 | 489.9 | [M + H]⁺ | 2 |
| 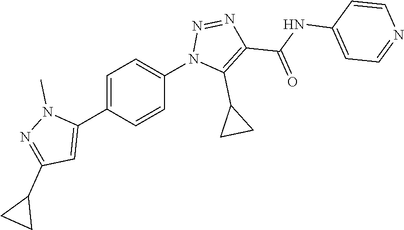 [SMILES: Cn1nc(C2CC2)cc1-c1ccc(-n2nnc(C(=O)Nc3ccncc3)c2C2CC2)cc1] | 7-109 | 11.31 | 425.5 | 425.8 | [M + H]⁺ | 2 |
| 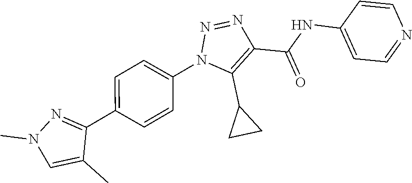 [SMILES: Cc1cn(C)nc1-c1ccc(-n2nnc(C(=O)Nc3ccncc3)c2C2CC2)cc1] | 7-110 | 10.8 | 399.5 | 400 | [M + H]⁺ | 2 |
| 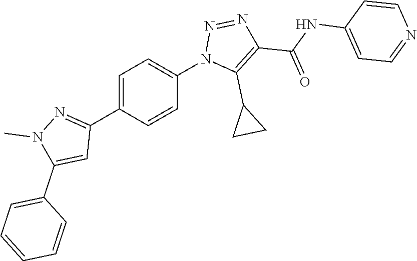 [SMILES: Cn1nc(-c2ccc(-n3nnc(C(=O)Nc4ccncc4)c3C3CC3)cc2)cc1-c1ccccc1] | 7-111 | 12.34 | 461.5 | 461.8 | [M + H]⁺ | 2 |
| 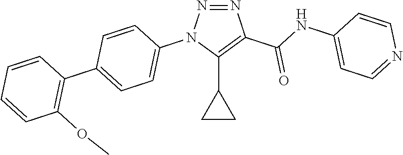 [SMILES: COc1ccccc1-c1ccc(-n2nnc(C(=O)Nc3ccncc3)c2C2CC2)cc1] | 7-112 | 11.99 | 411.5 | 411.8 | [M + H]⁺ | 2 |
| 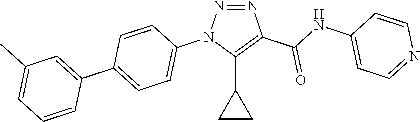 [SMILES: Cc1cccc(-c2ccc(-n3nnc(C(=O)Nc4ccncc4)c3C3CC3)cc2)c1] | 7-113 | 12.71 | 395.5 | 395.8 | [M + H]⁺ | 2 |
| 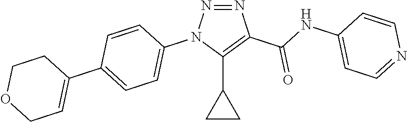 [SMILES: O=C(Nc1ccncc1)c1nnn(-c2ccc(C3=CCOCC3)cc2)c1C1CC1] | 7-114 | 10.69 | 387.4 | 388.4 | [M + H]⁺ | 2 |
| 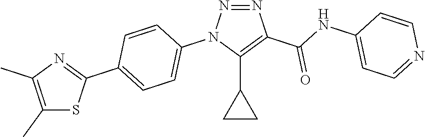 [SMILES: Cc1nc(-c2ccc(-n3nnc(C(=O)Nc4ccncc4)c3C3CC3)cc2)sc1C] | 7-115 | 11.98 | 416.5 | 417 | [M + H]⁺ | 2 |
| 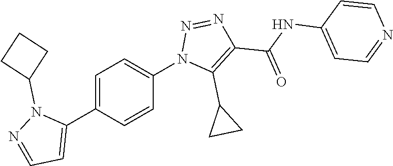 [SMILES: O=C(Nc1ccncc1)c1nnn(-c2ccc(-c3ccnn3C3CCC3)cc2)c1C1CC1] | 7-116 | 11.27 | 425.5 | 425.8 | [M + H]⁺ | 2 |
| 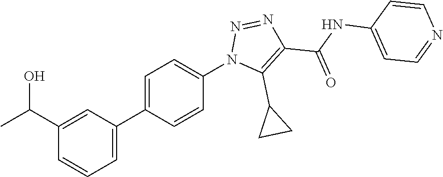 [SMILES: CC(O)c1cccc(-c2ccc(-n3nnc(C(=O)Nc4ccncc4)c3C3CC3)cc2)c1] | 7-117 | 11.26 | 425.5 | 426.1 | [M + H]⁺ | 2 |
| 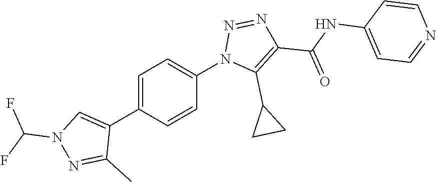 [SMILES: Cc1nn(C(F)F)cc1-c1ccc(-n2nnc(C(=O)Nc3ccncc3)c2C2CC2)cc1] | 7-118 | 10.63 | 435.4 | 435.9 | [M + H]⁺ | 2 |
| 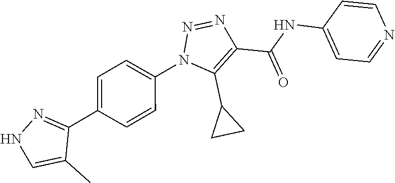 [SMILES: Cc1c[nH]nc1-c1ccc(-n2nnc(C(=O)Nc3ccncc3)c2C2CC2)cc1] | 7-119 | 10.47 | 385.4 | 386 | [M + H]⁺ | 2 |
| 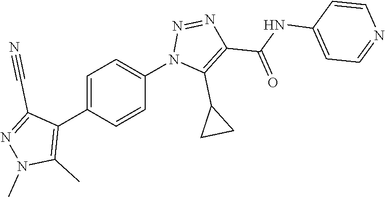 [SMILES: Cc1c(-c2ccc(-n3nnc(C(=O)Nc4ccncc4)c3C3CC3)cc2)c(C#N)nn1C] | 7-120 | 10.35 | 424.5 | 425 | [M + H]⁺ | 2 |
| 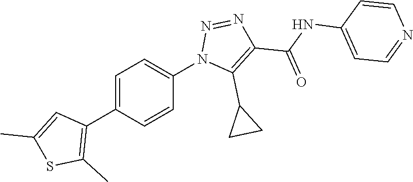 [SMILES: Cc1cc(-c2ccc(-n3nnc(C(=O)Nc4ccncc4)c3C3CC3)cc2)c(C)s1] | 7-121 | 13.12 | 415.5 | 416.2 | [M + H]⁺ | 2 |
| 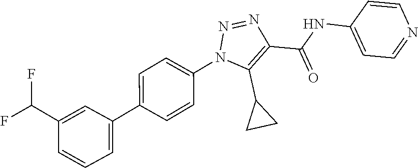 [SMILES: O=C(Nc1ccncc1)c1nnn(-c2ccc(-c3cccc(C(F)F)c3)cc2)c1C1CC1] | 7-122 | 11.83 | 431.4 | 431.9 | [M + H]⁺ | 2 |
| 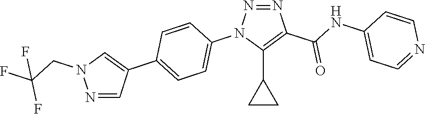 [SMILES: O=C(Nc1ccncc1)c1nnn(-c2ccc(-c3cnn(CC(F)(F)F)c3)cc2)c1C1CC1] | 7-123 | 10.8 | 453.4 | 454.1 | [M + H]⁺ | 2 |
| 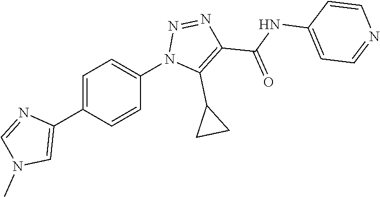 [SMILES: Cn1cnc(-c2ccc(-n3nnc(C(=O)Nc4ccncc4)c3C3CC3)cc2)c1] | 7-124 | 7.89 | 385.4 | 386 | [M + H]⁺ | 2 |
| 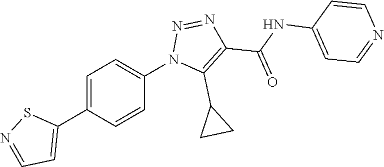 [SMILES: O=C(Nc1ccncc1)c1nnn(-c2ccc(-c3ccns3)cc2)c1C1CC1] | 7-125 | 10.6 | 388.4 | 389.3 | [M + H]⁺ | 2 |
| 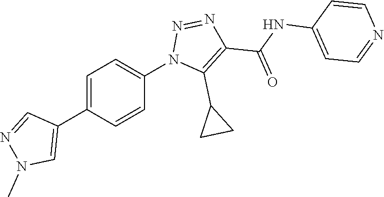 [SMILES: Cn1cc(-c2ccc(-n3nnc(C(=O)Nc4ccncc4)c3C3CC3)cc2)cn1] | 7-126 | 10.38 | 385.4 | 386 | [M + H]⁺ | 2 |
| 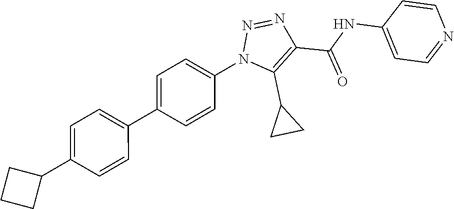 [SMILES: O=C(Nc1ccncc1)c1nnn(-c2ccc(-c3ccc(C4CCC4)cc3)cc2)c1C1CC1] | 7-127 | 13.79 | 435.5 | 436.1 | [M + H]⁺ | 2 |
| 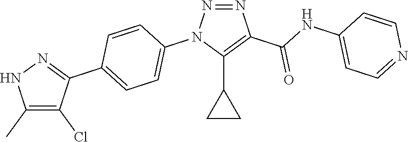 [SMILES: Cc1[nH]nc(-c2ccc(-n3nnc(C(=O)Nc4ccncc4)c3C3CC3)cc2)c1Cl] | 7-128 | 11.43 | 419.9 | 420 | [M + H]⁺ | 2 |
| 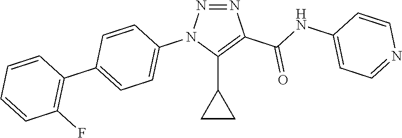 [SMILES: O=C(Nc1ccncc1)c1nnn(-c2ccc(-c3ccccc3F)cc2)c1C1CC1] | 7-129 | 11.93 | 399.4 | 399.8 | [M + H]⁺ | 2 |
| 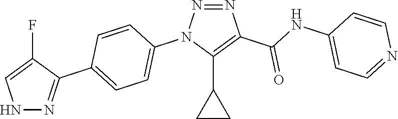 [SMILES: O=C(Nc1ccncc1)c1nnn(-c2ccc(-c3n[nH]cc3F)cc2)c1C1CC1] | 7-130 | 10.41 | 389.4 | 390.2 | [M + H]⁺ | 2 |
| 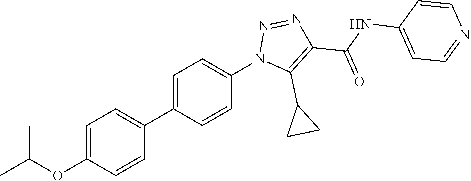 [SMILES: CC(C)Oc1ccc(-c2ccc(-n3nnc(C(=O)Nc4ccncc4)c3C3CC3)cc2)cc1] | 7-131 | 13.03 | 439.5 | 440.1 | [M + H]⁺ | 2 |
| 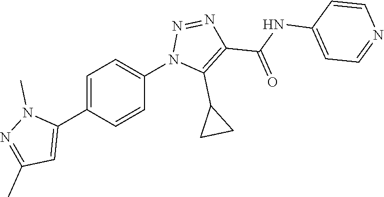 [SMILES: Cc1cc(-c2ccc(-n3nnc(C(=O)Nc4ccncc4)c3C3CC3)cc2)n(C)n1] | 7-132 | 10.55 | 399.5 | 400.1 | [M + H]⁺ | 2 |
| 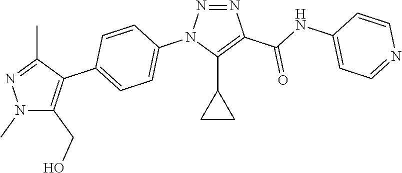 [SMILES: Cc1nn(C)c(CO)c1-c1ccc(-n2nnc(C(=O)Nc3ccncc3)c2C2CC2)cc1] | 7-133 | 9.86 | 429.5 | 430.3 | [M + H]⁺ | 2 |
| 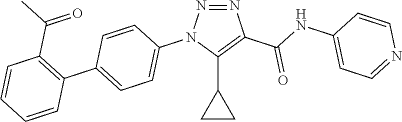 [SMILES: CC(=O)c1ccccc1-c1ccc(-n2nnc(C(=O)Nc3ccncc3)c2C2CC2)cc1] | 7-134 | 11.06 | 423.5 | 423.8 | [M + H]⁺ | 2 |
| 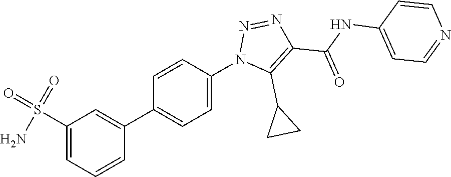 [SMILES: NS(=O)(=O)c1cccc(-c2ccc(-n3nnc(C(=O)Nc4ccncc4)c3C3CC3)cc2)c1] | 7-135 | 9.91 | 460.5 | 460.9 | [M + H]⁺ | 2 |
| 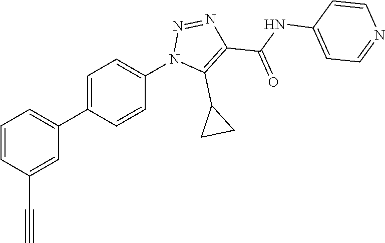 [SMILES: C#Cc1cccc(-c2ccc(-n3nnc(C(=O)Nc4ccncc4)c3C3CC3)cc2)c1] | 7-136 | 12.48 | 405.5 | 406.2 | [M + H]⁺ | 2 |
| 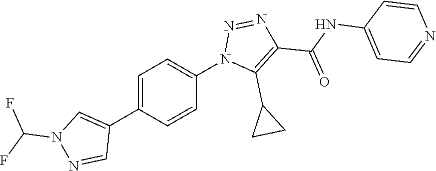 [SMILES: O=C(Nc1ccncc1)c1nnn(-c2ccc(-c3cnn(C(F)F)c3)cc2)c1C1CC1] | 7-137 | 10.31 | 421.4 | 422.1 | [M + H]⁺ | 2 |
| 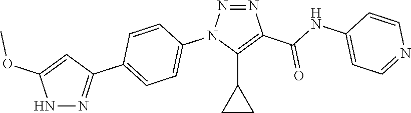 [SMILES: COc1cc(-c2ccc(-n3nnc(C(=O)Nc4ccncc4)c3C3CC3)cc2)n[nH]1] | 7-138 | 10.15 | 401.4 | 402.3 | [M + H]⁺ | 2 |
| 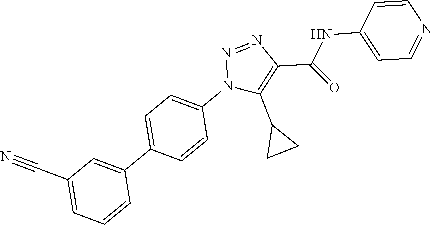 [SMILES: N#Cc1cccc(-c2ccc(-n3nnc(C(=O)Nc4ccncc4)c3C3CC3)cc2)c1] | 7-139 | 11.18 | 406.4 | 407 | [M + H]⁺ | 2 |
| 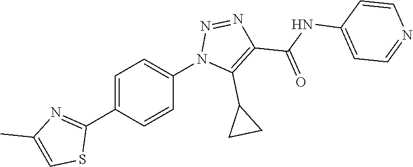 [SMILES: Cc1csc(-c2ccc(-n3nnc(C(=O)Nc4ccncc4)c3C3CC3)cc2)n1] | 7-140 | 11.14 | 402.5 | 403.1 | [M + H]⁺ | 2 |
| 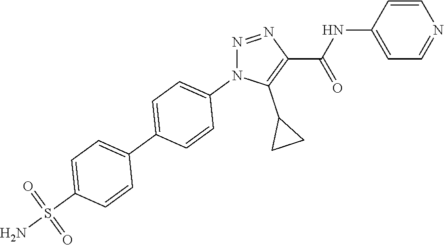 [SMILES: NS(=O)(=O)c1ccc(-c2ccc(-n3nnc(C(=O)Nc4ccncc4)c3C3CC3)cc2)cc1] | 7-141 | 9.74 | 460.5 | 460.9 | [M + H]⁺ | 2 |
| 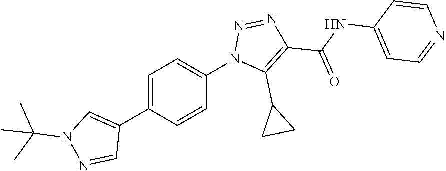 [SMILES: CC(C)(C)n1cc(-c2ccc(-n3nnc(C(=O)Nc4ccncc4)c3C3CC3)cc2)cn1] | 7-142 | 11.39 | 427.5 | 428.4 | [M + H]⁺ | 2 |
| 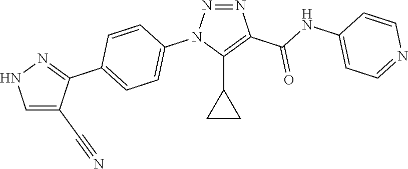 [SMILES: N#Cc1c[nH]nc1-c1ccc(-n2nnc(C(=O)Nc3ccncc3)c2C2CC2)cc1] | 7-143 | 9.81 | 396.4 | 396.8 | [M + H]⁺ | 2 |
| 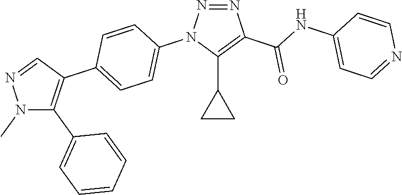 [SMILES: Cn1ncc(-c2ccc(-n3nnc(C(=O)Nc4ccncc4)c3C3CC3)cc2)c1-c1ccccc1] | 7-144 | 11.69 | 461.5 | 462 | [M + H]⁺ | 2 |
| 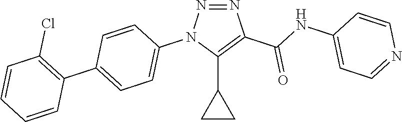 [SMILES: O=C(Nc1ccncc1)c1nnn(-c2ccc(-c3ccccc3Cl)cc2)c1C1CC1] | 7-145 | 12.27 | 415.9 | 416.1 | [M + H]⁺ | 2 |
| 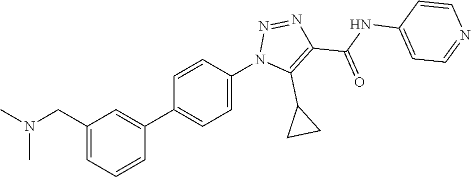 [SMILES: CN(C)Cc1cccc(-c2ccc(-n3nnc(C(=O)Nc4ccncc4)c3C3CC3)cc2)c1] | 7-146 | 9.34 | 438.5 | 438.6 | [M + H]⁺ | 2 |
| 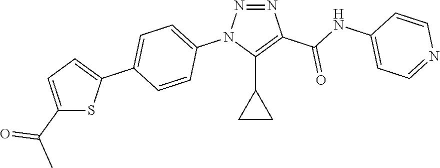 [SMILES: CC(=O)c1ccc(-c2ccc(-n3nnc(C(=O)Nc4ccncc4)c3C3CC3)cc2)s1] | 7-147 | 11.36 | 429.5 | 430.2 | [M + H]⁺ | 2 |
| 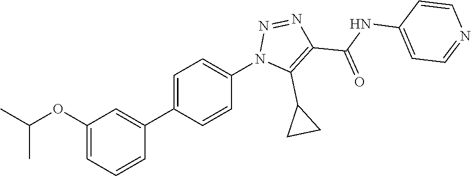 [SMILES: CC(C)Oc1cccc(-c2ccc(-n3nnc(C(=O)Nc4ccncc4)c3C3CC3)cc2)c1] | 7-148 | 13.01 | 439.5 | 440.1 | [M + H]⁺ | 2 |
| 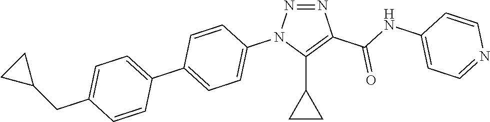 [SMILES: O=C(Nc1ccncc1)c1nnn(-c2ccc(-c3ccc(CC4CC4)cc3)cc2)c1C1CC1] | 7-149 | 13.39 | 435.5 | 436.1 | [M + H]⁺ | 2 |
| 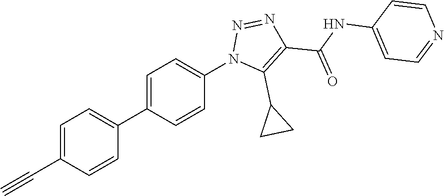 [SMILES: C#Cc1ccc(-c2ccc(-n3nnc(C(=O)Nc4ccncc4)c3C3CC3)cc2)cc1] | 7-150 | 12.37 | 405.5 | 405.8 | [M + H]⁺ | 2 |
| 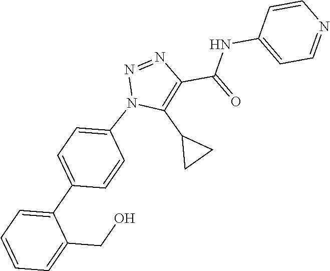 [SMILES: O=C(Nc1ccncc1)c1nnn(-c2ccc(-c3ccccc3CO)cc2)c1C1CC1] | 7-151 | 11.17 | 411.5 | 412.1 | [M + H]⁺ | 2 |
| 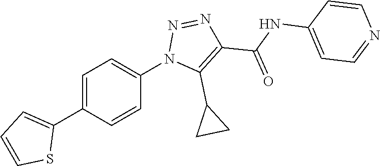 [SMILES: O=C(Nc1ccncc1)c1nnn(-c2ccc(-c3cccs3)cc2)c1C1CC1] | 7-152 | 11.82 | 387.5 | 388.3 | [M + H]⁺ | 2 |
| 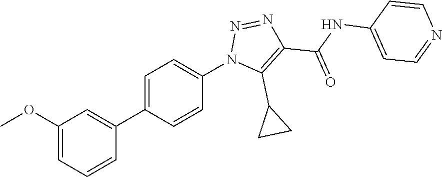 [SMILES: COc1cccc(-c2ccc(-n3nnc(C(=O)Nc4ccncc4)c3C3CC3)cc2)c1] | 7-153 | 12.19 | 411.5 | 411.9 | [M + H]⁺ | 2 |
| 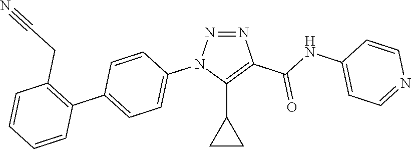 [SMILES: N#CCc1ccccc1-c1ccc(-n2nnc(C(=O)Nc3ccncc3)c2C2CC2)cc1] | 7-154 | 11.19 | 420.5 | 421.1 | [M + H]⁺ | 2 |
| 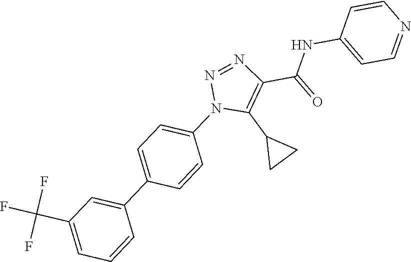 [SMILES: O=C(Nc1ccncc1)c1nnn(-c2ccc(-c3cccc(C(F)(F)F)c3)cc2)c1C1CC1] | 7-155 | 12.71 | 449.4 | 450 | [M + H]⁺ | 2 |
| 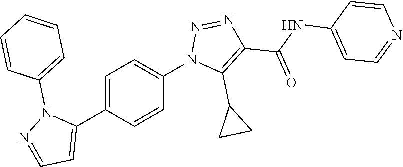 [SMILES: O=C(Nc1ccncc1)c1nnn(-c2ccc(-c3ccnn3-c3ccccc3)cc2)c1C1CC1] | 7-156 | 11.19 | 447.5 | 447.7 | [M + H]⁺ | 2 |
| 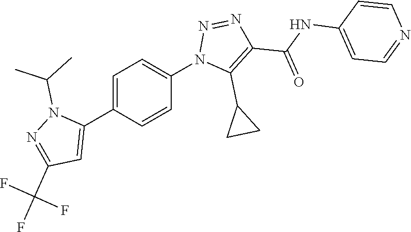 [SMILES: CC(C)n1nc(C(F)(F)F)cc1-c1ccc(-n2nnc(C(=O)Nc3ccncc3)c2C2CC2)cc1] | 7-157 | 11.93 | 481.5 | 482.3 | [M + H]⁺ | 2 |
| 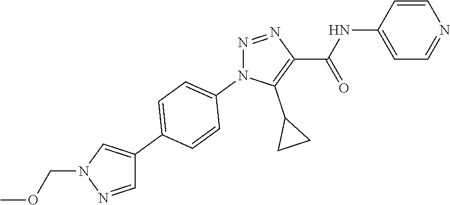 [SMILES: COCn1cc(-c2ccc(-n3nnc(C(=O)Nc4ccncc4)c3C3CC3)cc2)cn1] | 7-158 | 10.12 | 415.5 | 416.3 | [M + H]⁺ | 2 |
| 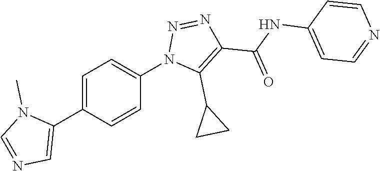 [SMILES: Cn1cncc1-c1ccc(-n2nnc(C(=O)Nc3ccncc3)c2C2CC2)cc1] | 7-159 | 7.6 | 385.4 | 385.9 | [M + H]⁺ | 2 |
| 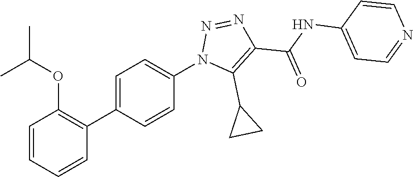 [SMILES: CC(C)Oc1ccccc1-c1ccc(-n2nnc(C(=O)Nc3ccncc3)c2C2CC2)cc1] | 7-160 | 13.02 | 439.5 | 440.1 | [M + H]⁺ | 2 |
| 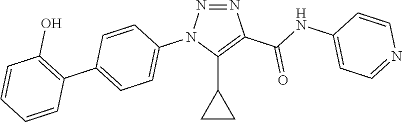 [SMILES: O=C(Nc1ccncc1)c1nnn(-c2ccc(-c3ccccc3O)cc2)c1C1CC1] | 7-161 | 11.52 | 397.4 | 397.9 | [M + H]⁺ | 2 |
| 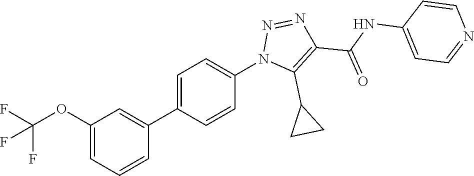 [SMILES: O=C(Nc1ccncc1)c1nnn(-c2ccc(-c3cccc(OC(F)(F)F)c3)cc2)c1C1CC1] | 7-162 | 12.99 | 465.4 | 465.8 | [M + H]⁺ | 2 |
| 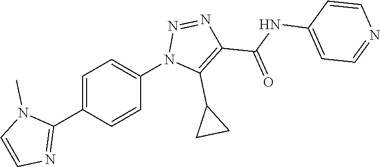 [SMILES: Cn1ccnc1-c1ccc(-n2nnc(C(=O)Nc3ccncc3)c2C2CC2)cc1] | 7-163 | 7.1 | 385.4 | 386.1 | [M + H]⁺ | 2 |
| 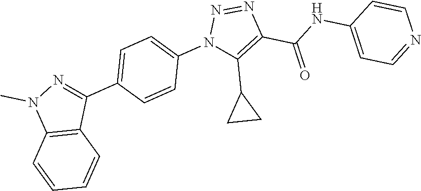 [SMILES: Cn1nc(-c2ccc(-n3nnc(C(=O)Nc4ccncc4)c3C3CC3)cc2)c2ccccc21] | 7-164 | 11.96 | 435.5 | 436.1 | [M + H]⁺ | 2 |
| 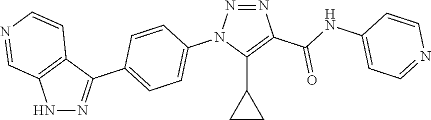 [SMILES: O=C(Nc1ccncc1)c1nnn(-c2ccc(-c3n[nH]c4cnccc34)cc2)c1C1CC1] | 7-165 | 8.62 | 422.5 | 422.8 | [M + H]⁺ | 2 |
| 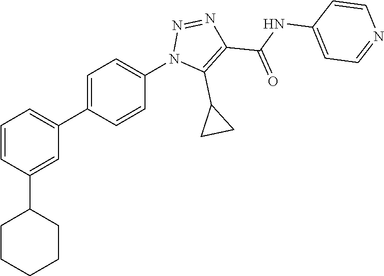 [SMILES: O=C(Nc1ccncc1)c1nnn(-c2ccc(-c3cccc(C4CCCCC4)c3)cc2)c1C1CC1] | 7-166 | 14.33 | 463.6 | 463.8 | [M + H]⁺ | 2 |
| 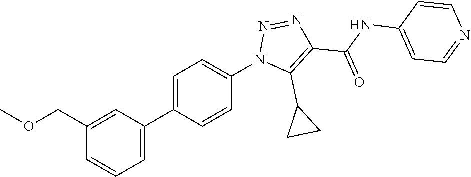 [SMILES: COCc1cccc(-c2ccc(-n3nnc(C(=O)Nc4ccncc4)c3C3CC3)cc2)c1] | 7-167 | 11.98 | 425.5 | 425.9 | [M + H]⁺ | 2 |
| 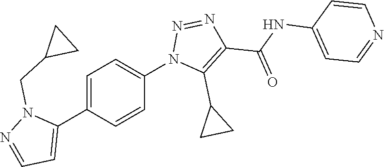 [SMILES: O=C(Nc1ccncc1)c1nnn(-c2ccc(-c3ccnn3CC3CC3)cc2)c1C1CC1] | 7-168 | 10.97 | 425.5 | 426 | [M + H]⁺ | 2 |
| 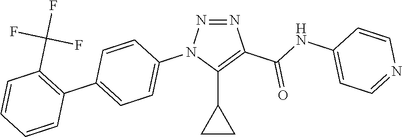 [SMILES: O=C(Nc1ccncc1)c1nnn(-c2ccc(-c3ccccc3C(F)(F)F)cc2)c1C1CC1] | 7-169 | 12.42 | 449.4 | 449.7 | [M + H]⁺ | 2 |
| 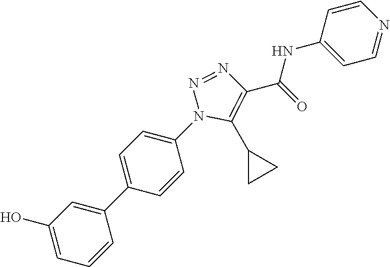 [SMILES: O=C(Nc1ccncc1)c1nnn(-c2ccc(-c3cccc(O)c3)cc2)c1C1CC1] | 7-170 | 11.06 | 397.4 | 397.9 | [M + H]⁺ | 2 |
| 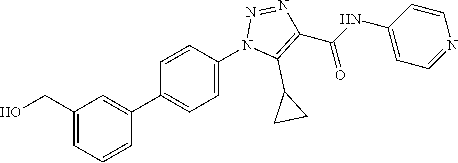 [SMILES: O=C(Nc1ccncc1)c1nnn(-c2ccc(-c3cccc(CO)c3)cc2)c1C1CC1] | 7-171 | 10.97 | 411.5 | 412 | [M + H]⁺ | 2 |
| 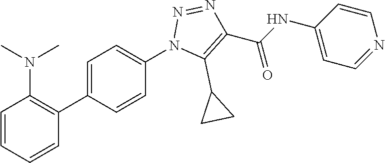 [SMILES: CN(C)c1ccccc1-c1ccc(-n2nnc(C(=O)Nc3ccncc3)c2C2CC2)cc1] | 7-172 | 10.26 | 424.5 | 424.9 | [M + H]⁺ | 2 |
| 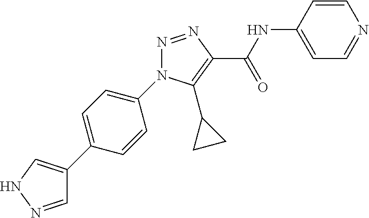 [SMILES: O=C(Nc1ccncc1)c1nnn(-c2ccc(-c3cn[nH]c3)cc2)c1C1CC1] | 7-173 | 9.55 | 371.4 | 371.7 | [M + H]⁺ | 2 |
| 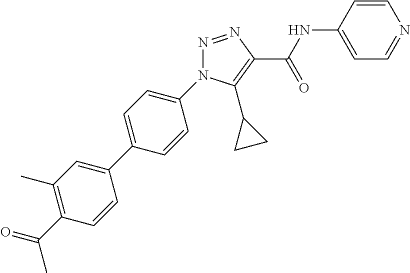 [SMILES: CC(=O)c1ccc(-c2ccc(-n3nnc(C(=O)Nc4ccncc4)c3C3CC3)cc2)cc1C] | 7-174 | 11.82 | 437.5 | 438 | [M + H]⁺ | 2 |
| 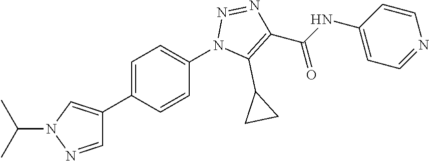 [SMILES: CC(C)n1cc(-c2ccc(-n3nnc(C(=O)Nc4ccncc4)c3C3CC3)cc2)cn1] | 7-175 | 10.73 | 413.5 | 414.2 | [M + H]⁺ | 2 |
| 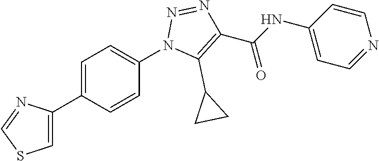 [SMILES: O=C(Nc1ccncc1)c1nnn(-c2ccc(-c3cscn3)cc2)c1C1CC1] | 7-176 | 10.42 | 388.4 | 389.3 | [M + H]⁺ | 2 |
| 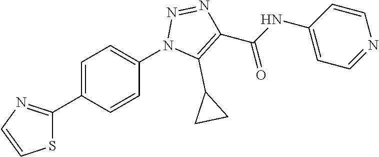 [SMILES: O=C(Nc1ccncc1)c1nnn(-c2ccc(-c3nccs3)cc2)c1C1CC1] | 7-177 | 10.52 | 388.4 | 389 | [M + H]⁺ | 2 |
| 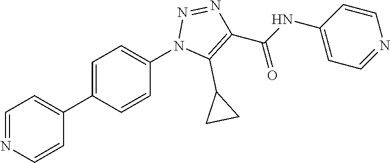 [SMILES: O=C(Nc1ccncc1)c1nnn(-c2ccc(-c3ccncc3)cc2)c1C1CC1] | 7-178 | 7.94 | 382.4 | 383 | [M + H]⁺ | 2 |
| 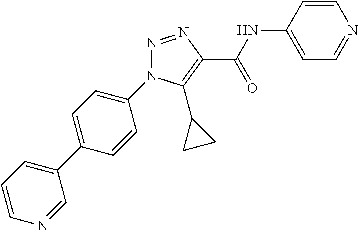 [SMILES: O=C(Nc1ccncc1)c1nnn(-c2ccc(-c3cccnc3)cc2)c1C1CC1] | 7-179 | 8.09 | 382.4 | 383 | [M + H]⁺ | 2 |
| 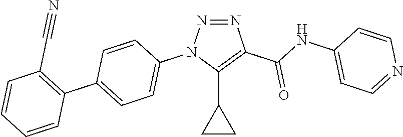 [SMILES: N#Cc1ccccc1-c1ccc(-n2nnc(C(=O)Nc3ccncc3)c2C2CC2)cc1] | 7-180 | 11.01 | 406.4 | 407.1 | [M + H]⁺ | 2 |
| 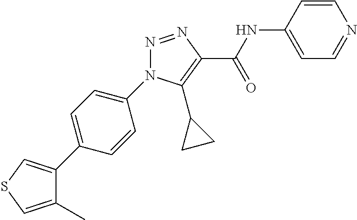 [SMILES: Cc1cscc1-c1ccc(-n2nnc(C(=O)Nc3ccncc3)c2C2CC2)cc1] | 7-181 | 12.37 | 401.5 | 402.3 | [M + H]⁺ | 2 |
| 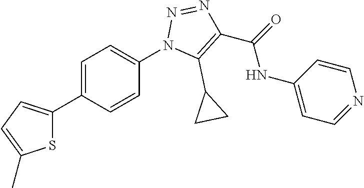 [SMILES: Cc1ccc(-c2ccc(-n3nnc(C(=O)Nc4ccncc4)c3C3CC3)cc2)s1] | 7-182 | 12.69 | 401.5 | 402.4 | [M + H]⁺ | 2 |
| 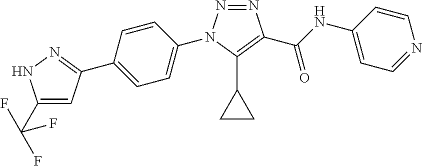 [SMILES: O=C(Nc1ccncc1)c1nnn(-c2ccc(-c3cc(C(F)(F)F)[nH]n3)cc2)c1C1CC1] | 7-183 | 11.28 | 439.4 | 440.1 | [M + H]⁺ | 2 |
| 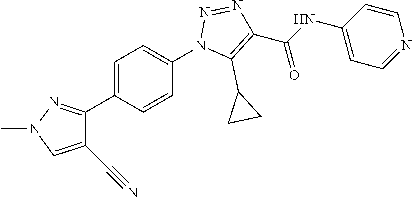 [SMILES: Cn1cc(C#N)c(-c2ccc(-n3nnc(C(=O)Nc4ccncc4)c3C3CC3)cc2)n1] | 7-184 | 10.33 | 410.4 | 411.1 | [M + H]⁺ | 2 |
| 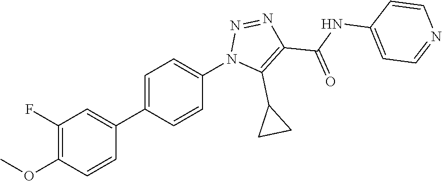 [SMILES: COc1ccc(-c2ccc(-n3nnc(C(=O)Nc4ccncc4)c3C3CC3)cc2)cc1F] | 7-185 | 12.09 | 429.5 | 429.9 | [M + H]⁺ | 2 |
| 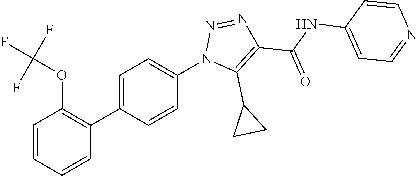 [SMILES: O=C(Nc1ccncc1)c1nnn(-c2ccc(-c3ccccc3OC(F)(F)F)cc2)c1C1CC1] | 7-186 | 12.71 | 465.4 | 465.9 | [M + H]⁺ | 2 |
| 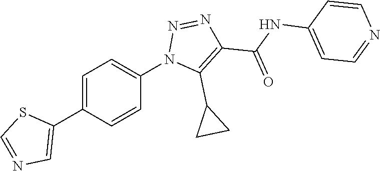 [SMILES: O=C(Nc1ccncc1)c1nnn(-c2ccc(-c3cncs3)cc2)c1C1CC1] | 7-187 | 10.01 | 388.4 | 389.1 | [M + H]⁺ | 2 |
| 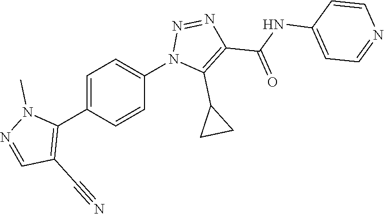 [SMILES: Cn1ncc(C#N)c1-c1ccc(-n2nnc(C(=O)Nc3ccncc3)c2C2CC2)cc1] | 7-188 | 9.51 | 410.4 | 411.1 | [M + H]⁺ | 2 |
| 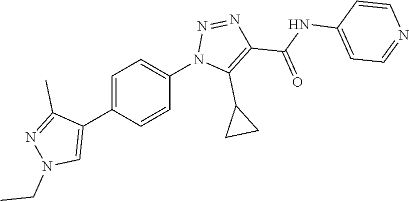 [SMILES: CCn1cc(-c2ccc(-n3nnc(C(=O)Nc4ccncc4)c3C3CC3)cc2)c(C)n1] | 7-189 | 10.75 | 413.5 | 414.4 | [M + H]⁺ | 2 |
| 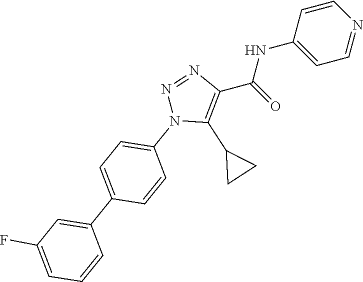 [SMILES: O=C(Nc1ccncc1)c1nnn(-c2ccc(-c3cccc(F)c3)cc2)c1C1CC1] | 7-190 | 12.16 | 399.4 | 400 | [M + H]⁺ | 2 |
| 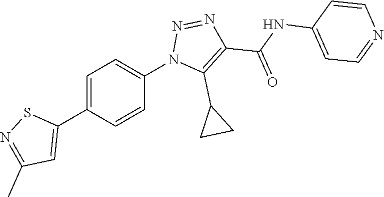 [SMILES: Cc1cc(-c2ccc(-n3nnc(C(=O)Nc4ccncc4)c3C3CC3)cc2)sn1] | 7-191 | 11.14 | 402.5 | 403.3 | [M + H]⁺ | 2 |
| 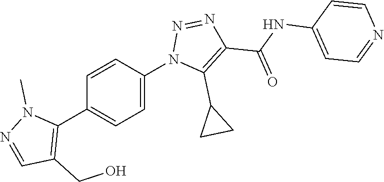 [SMILES: Cn1ncc(CO)c1-c1ccc(-n2nnc(C(=O)Nc3ccncc3)c2C2CC2)cc1] | 7-192 | 9.02 | 415.5 | 415.9 | [M + H]⁺ | 2 |
| 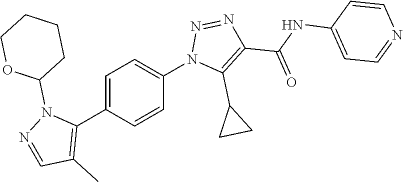 [SMILES: Cc1cnn(C2CCCCO2)c1-c1ccc(-n2nnc(C(=O)Nc3ccncc3)c2C2CC2)cc1] | 7-193 | 11.1 | 469.5 | 469.9 | [M + H]⁺ | 2 |
| 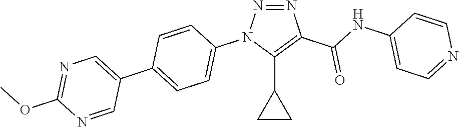 [SMILES: COc1ncc(-c2ccc(-n3nnc(C(=O)Nc4ccncc4)c3C3CC3)cc2)cn1] | 7-194 | 10.46 | 413.4 | 414.2 | [M + H]⁺ | 2 |
| 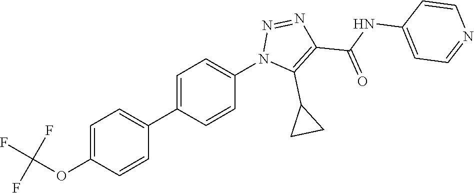 [SMILES: O=C(Nc1ccncc1)c1nnn(-c2ccc(-c3ccc(OC(F)(F)F)cc3)cc2)c1C1CC1] | 7-195 | 12.99 | 465.4 | 465.9 | [M + H]⁺ | 2 |
| 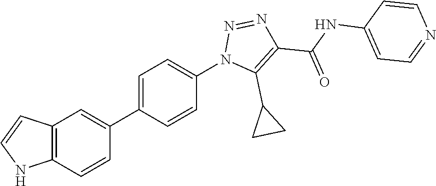 [SMILES: O=C(Nc1ccncc1)c1nnn(-c2ccc(-c3ccc4[nH]ccc4c3)cc2)c1C1CC1] | 7-196 | 11.86 | 420.5 | 420.9 | [M + H]⁺ | 2 |
| 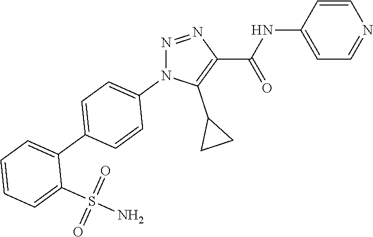 [SMILES: NS(=O)(=O)c1ccccc1-c1ccc(-n2nnc(C(=O)Nc3ccncc3)c2C2CC2)cc1] | 7-197 | 10.17 | 460.5 | 460.9 | [M + H]⁺ | 2 |
| 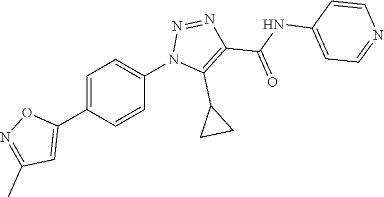 [SMILES: Cc1cc(-c2ccc(-n3nnc(C(=O)Nc4ccncc4)c3C3CC3)cc2)on1] | 7-198 | 10.46 | 386.4 | 387 | [M + H]⁺ | 2 |
| 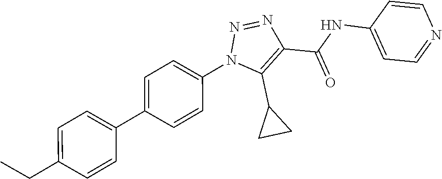 [SMILES: CCc1ccc(-c2ccc(-n3nnc(C(=O)Nc4ccncc4)c3C3CC3)cc2)cc1] | 7-199 | 13.13 | 409.5 | 410 | [M + H]⁺ | 2 |
| 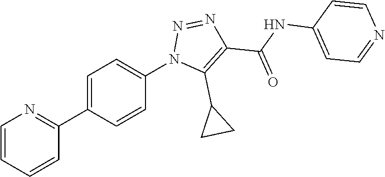 [SMILES: O=C(Nc1ccncc1)c1nnn(-c2ccc(-c3ccccn3)cc2)c1C1CC1] | 7-200 | 9.26 | 382.4 | 383 | [M + H]⁺ | 2 |
| 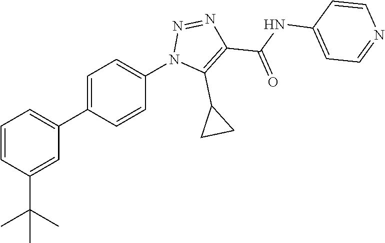 [SMILES: CC(C)(C)c1cccc(-c2ccc(-n3nnc(C(=O)Nc4ccncc4)c3C3CC3)cc2)c1] | 7-201 | 13.77 | 437.5 | 438.2 | [M + H]⁺ | 2 |
| 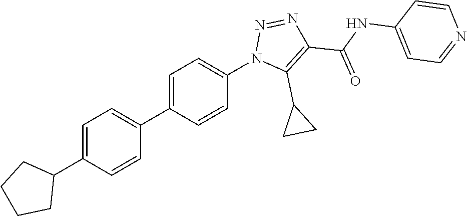 [SMILES: O=C(Nc1ccncc1)c1nnn(-c2ccc(-c3ccc(C4CCCC4)cc3)cc2)c1C1CC1] | 7-202 | 14.21 | 449.6 | 450 | [M + H]⁺ | 2 |
| 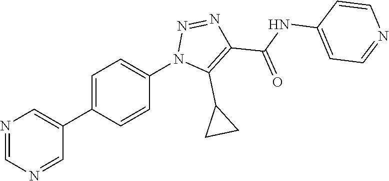 [SMILES: O=C(Nc1ccncc1)c1nnn(-c2ccc(-c3cncnc3)cc2)c1C1CC1] | 7-203 | 9.62 | 383.4 | 384.1 | [M + H]⁺ | 2 |
| 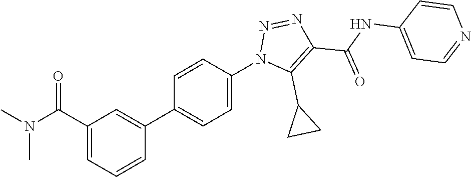 [SMILES: CN(C)C(=O)c1cccc(-c2ccc(-n3nnc(C(=O)Nc4ccncc4)c3C3CC3)cc2)c1] | 7-204 | 10.83 | 452.5 | 452.9 | [M + H]⁺ | 2 |
| 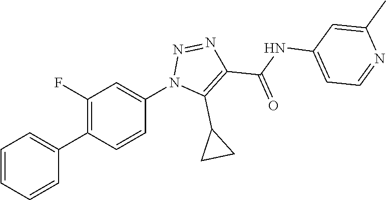 [SMILES: Cc1cc(NC(=O)c2nnn(-c3ccc(-c4ccccc4)c(F)c3)c2C2CC2)ccn1] | 7-205 | 12.13 | 413.5 | 414.2 | [M + H]⁺ | 2 |
| 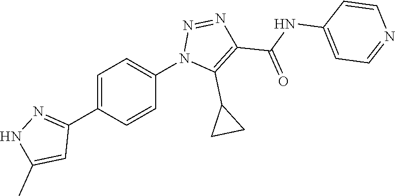 [SMILES: Cc1cc(-c2ccc(-n3nnc(C(=O)Nc4ccncc4)c3C3CC3)cc2)n[nH]1] | 7-206 | 10.4 | 385.4 | 385.7 | [M + H]⁺ | 2 |
| 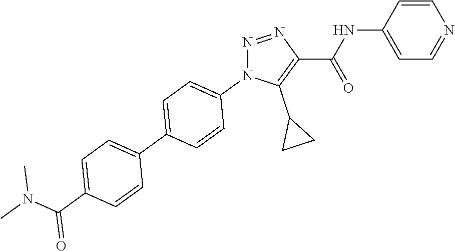 [SMILES: CN(C)C(=O)c1ccc(-c2ccc(-n3nnc(C(=O)Nc4ccncc4)c3C3CC3)cc2)cc1] | 7-207 | 10.72 | 452.5 | 452.9 | [M + H]⁺ | 2 |
| 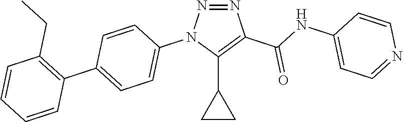 [SMILES: CCc1ccccc1-c1ccc(-n2nnc(C(=O)Nc3ccncc3)c2C2CC2)cc1] | 7-208 | 13.04 | 409.5 | 410 | [M + H]⁺ | 2 |
| 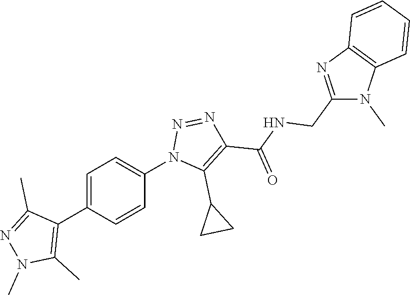 [SMILES: Cc1nn(C)c(C)c1-c1ccc(-n2nnc(C(=O)NCc3nc4ccccc4n3C)c2C2CC2)cc1] | 7-209 | 10.61 | 480.6 | 481.3 | [M + H]⁺ | 2 |
| 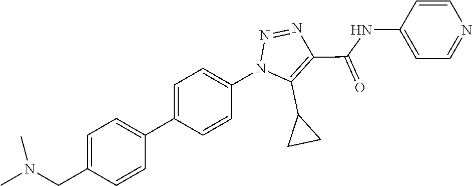 [SMILES: CN(C)Cc1ccc(-c2ccc(-n3nnc(C(=O)Nc4ccncc4)c3C3CC3)cc2)cc1] | 7-210 | 9.2 | 438.5 | 439.2 | [M + H]⁺ | 2 |
| 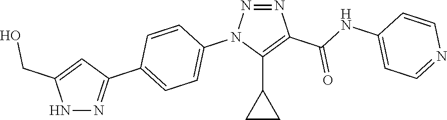 [SMILES: O=C(Nc1ccncc1)c1nnn(-c2ccc(-c3cc(CO)[nH]n3)cc2)c1C1CC1] | 7-211 | 9.1 | 401.4 | 401.9 | [M + H]⁺ | 2 |
| 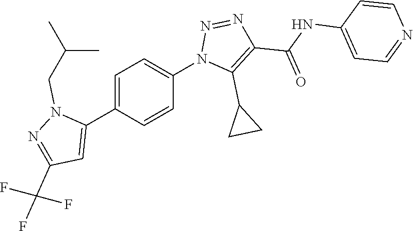 [SMILES: CC(C)Cn1nc(C(F)(F)F)cc1-c1ccc(-n2nnc(C(=O)Nc3ccncc3)c2C2CC2)cc1] | 7-212 | 11.47 | 495.5 | 496.1 | [M + H]⁺ | 2 |
| 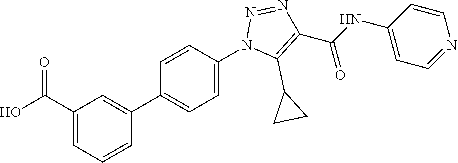 [SMILES: O=C(O)c1cccc(-c2ccc(-n3nnc(C(=O)Nc4ccncc4)c3C3CC3)cc2)c1] | 7-213 | 11.46 | 425.4 | 426 | [M + H]⁺ | 2 |
| 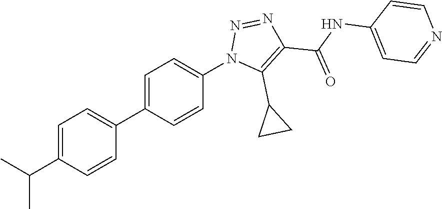 [SMILES: CC(C)c1ccc(-c2ccc(-n3nnc(C(=O)Nc4ccncc4)c3C3CC3)cc2)cc1] | 7-214 | 13.65 | 423.5 | 424.1 | [M + H]⁺ | 2 |
| 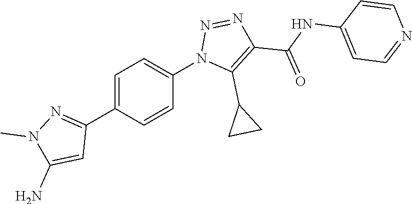 [SMILES: Cn1nc(-c2ccc(-n3nnc(C(=O)Nc4ccncc4)c3C3CC3)cc2)cc1N] | 7-215 | 8.69 | 400,4 | 401.1 | [M + H]⁺ | 2 |
| 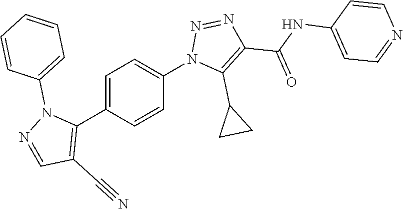 [SMILES: N#Cc1cnn(-c2ccccc2)c1-c1ccc(-n2nnc(C(=O)Nc3ccncc3)c2C2CC2)cc1] | 7-216 | 11.06 | 472.5 | 472.8 | [M + H]⁺ | 2 |
| 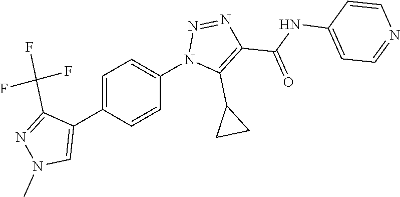 [SMILES: Cn1cc(-c2ccc(-n3nnc(C(=O)Nc4ccncc4)c3C3CC3)cc2)c(C(F)(F)F)n1] | 7-217 | 11.07 | 453.4 | 454.1 | [M + H]⁺ | 2 |
| 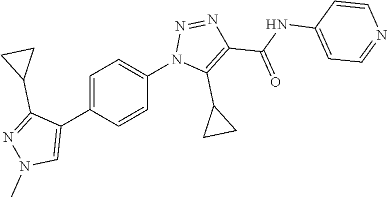 [SMILES: Cn1cc(-c2ccc(-n3nnc(C(=O)Nc4ccncc4)c3C3CC3)cc2)c(C2CC2)n1] | 7-218 | 11.01 | 425.5 | 426.1 | [M + H]⁺ | 2 |
| 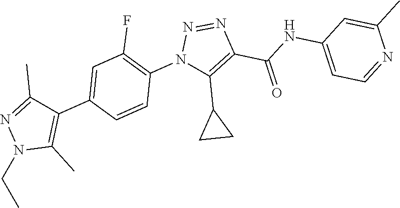 [SMILES: CCn1nc(C)c(-c2ccc(-n3nnc(C(=O)Nc4ccnc(C)c4)c3C3CC3)c(F)c2)c1C] | 7-219 | 11.08 | 459.5 | 460 | [M + H]⁺ | 2 |
| 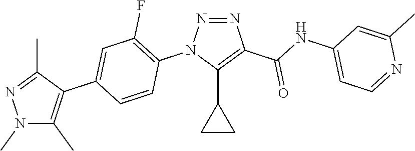 | 7-220 | 11 | 445.5 | 445.9 | [M + H]⁺ | 2 |
| 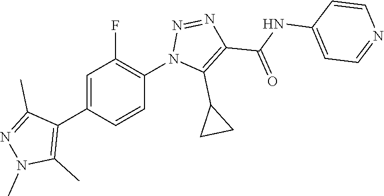 [SMILES: Cc1nn(C)c(C)c1-c1ccc(-n2nnc(C(=O)Nc3ccncc3)c2C2CC2)c(F)c1] | 7-221 | 10.76 | 431.5 | 432 | [M + H]⁺ | 2 |
| 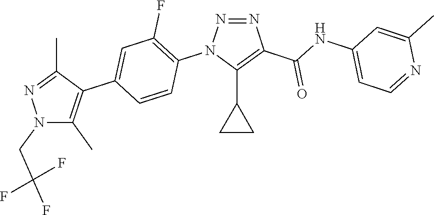 [SMILES: Cc1cc(NC(=O)c2nnn(-c3ccc(-c4c(C)nn(CC(F)(F)F)c4C)cc3F)c2C2CC2)ccn1] | 7-222 | 11.59 | 513.5 | 513.7 | [M + H]⁺ | 2 |
| 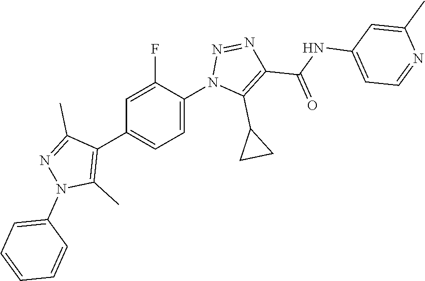 [SMILES: Cc1cc(NC(=O)c2nnn(-c3ccc(-c4c(C)nn(-c5ccccc5)c4C)cc3F)c2C2CC2)ccn1] | 7-223 | 12.28 | 507.6 | 508 | [M + H]⁺ | 2 |
| 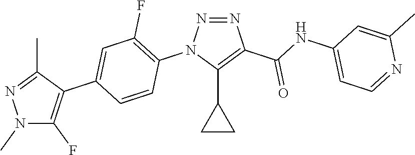 | 7-224 | 11.44 | 449.5 | 450 | [M + H]⁺ | 2 |
| 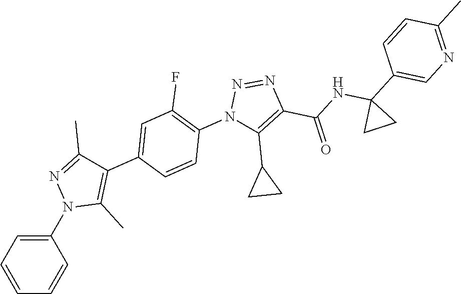 [SMILES: Cc1ccc(C2(NC(=O)c3nnn(-c4ccc(-c5c(C)nn(-c6ccccc6)c5C)cc4F)c3C3CC3)CC2)cn1] | 7-225 | 11.72 | 547.6 | 548 | [M + H]⁺ | 2 |
| 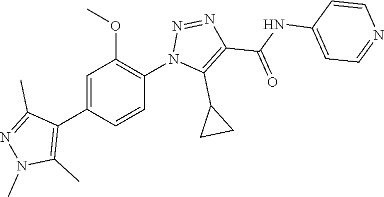 [SMILES: COc1cc(-c2c(C)nn(C)c2C)ccc1-n1nnc(C(=O)Nc2ccncc2)c1C1CC1] | 7-226 | 10.29 | 443.5 | 444 | [M + H]⁺ | 2 |
| 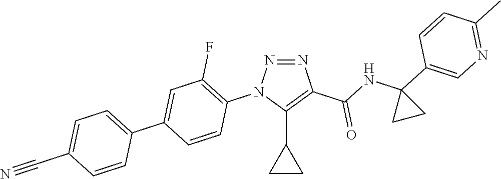 [SMILES: Cc1ccc(C2(NC(=O)c3nnn(-c4ccc(-c5ccc(C#N)cc5)cc4F)c3C3CC3)CC2)cn1] | 7-227 | 10.86 | 478.5 | 478.9 | [M + H]⁺ | 2 |
| 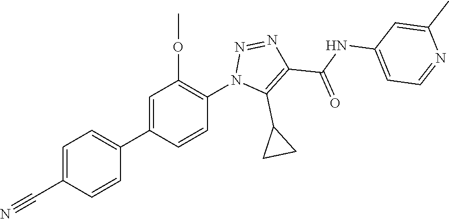 [SMILES: COc1cc(-c2ccc(C#N)cc2)ccc1-n1nnc(C(=O)Nc2ccnc(C)c2)c1C1CC1] | 7-228 | 10.18 | 450.5 | 450.8 | [M + H]⁺ | 2 |
| 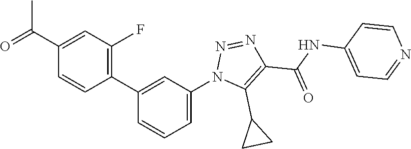 [SMILES: CC(=O)c1ccc(-c2cccc(-n3nnc(C(=O)Nc4ccncc4)c3C3CC3)c2)c(F)c1] | 7-229 | 11.63 | 441.5 | 442.3 | [M + H]⁺ | 2 |
| 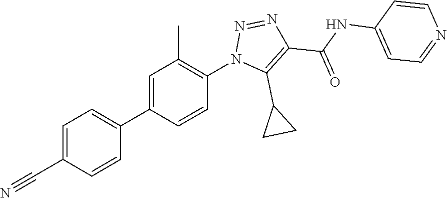 [SMILES: Cc1cc(-c2ccc(C#N)cc2)ccc1-n1nnc(C(=O)Nc2ccncc2)c1C1CC1] | 7-230 | 11.53 | 420.5 | 420.8 | [M + H]⁺ | 2 |
| 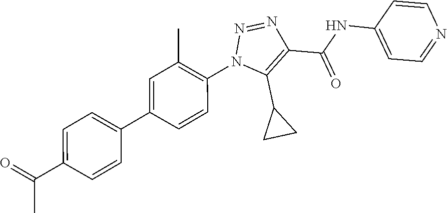 [SMILES: CC(=O)c1ccc(-c2ccc(-n3nnc(C(=O)Nc4ccncc4)c3C3CC3)c(C)c2)cc1] | 7-231 | 11.71 | 437.5 | 438.1 | [M + H]⁺ | 2 |
| 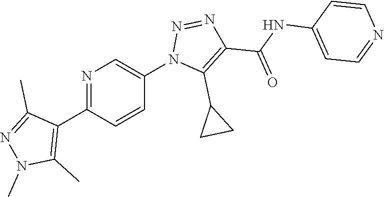 [SMILES: Cc1nn(C)c(C)c1-c1ccc(-n2nnc(C(=O)Nc3ccncc3)c2C2CC2)cn1] | 7-232 | 9.53 | 414.5 | 415.1 | [M + H]⁺ | 2 |
| 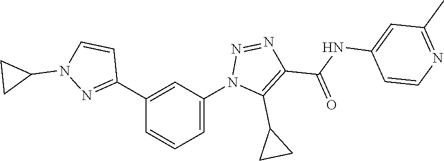 [SMILES: Cc1cc(NC(=O)c2nnn(-c3cccc(-c4ccn(C5CC5)n4)c3)c2C2CC2)ccn1] | 7-233 | 11 | 425.5 | 425.9 | [M + H]⁺ | 2 |
| 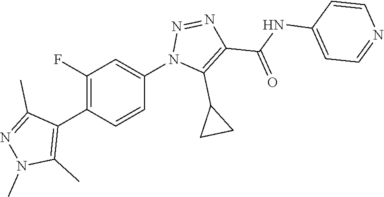 [SMILES: Cc1nn(C)c(C)c1-c1ccc(-n2nnc(C(=O)Nc3ccncc3)c2C2CC2)cc1F] | 7-234 | 10.78 | 431.5 | 432.1 | [M + H]⁺ | 2 |
| 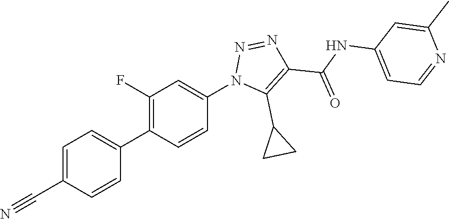 [SMILES: Cc1cc(NC(=O)c2nnn(-c3ccc(-c4ccc(C#N)cc4)c(F)c3)c2C2CC2)ccn1] | 7-235 | 10.46 | 438.5 | 439 | [M + H]⁺ | 2 |
| 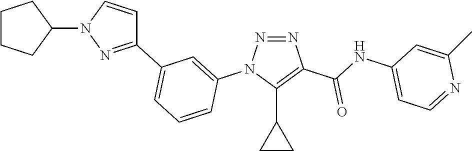 [SMILES: Cc1cc(NC(=O)c2nnn(-c3cccc(-c4ccn(C5CCCC5)n4)c3)c2C2CC2)ccn1] | 7-236 | 12.28 | 453.6 | 454.1 | [M + H]⁺ | 2 |
| 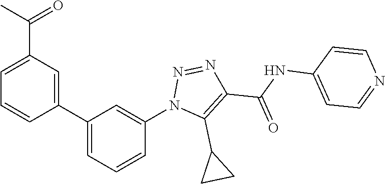 [SMILES: CC(=O)c1cccc(-c2cccc(-n3nnc(C(=O)Nc4ccncc4)c3C3CC3)c2)c1] | 7-237 | 11.42 | 423.5 | 424 | [M + H]⁺ | 2 |
| 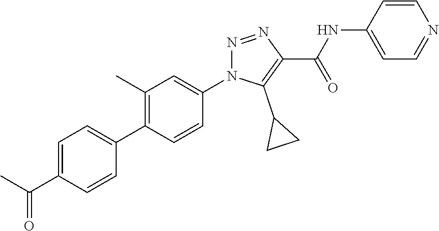 [SMILES: CC(=O)c1ccc(-c2ccc(-n3nnc(C(=O)Nc4ccncc4)c3C3CC3)cc2C)cc1] | 7-238 | 11.73 | 437.5 | 438.2 | [M + H]⁺ | 2 |
| 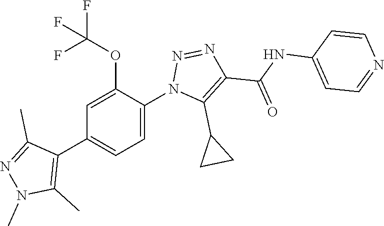 [SMILES: Cc1nn(C)c(C)c1-c1ccc(-n2nnc(C(=O)Nc3ccncc3)c2C2CC2)c(OC(F)(F)F)c1] | 7-239 | 11.48 | 497.5 | 497.8 | [M + H]⁺ | 2 |
| 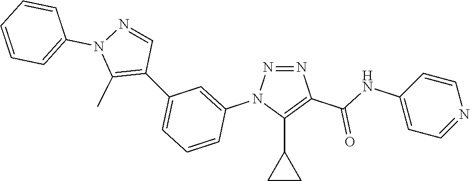 [SMILES: Cc1c(-c2cccc(-n3nnc(C(=O)Nc4ccncc4)c3C3CC3)c2)cnn1-c1ccccc1] | 7-240 | 11.56 | 461.5 | 462.1 | [M + H]⁺ | 2 |
| 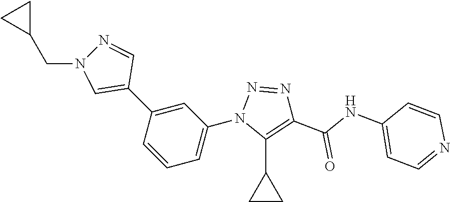 [SMILES: O=C(Nc1ccncc1)c1nnn(-c2cccc(-c3cnn(CC4CC4)c3)c2)c1C1CC1] | 7-241 | 11.25 | 425.5 | 426.3 | [M + H]⁺ | 2 |
| 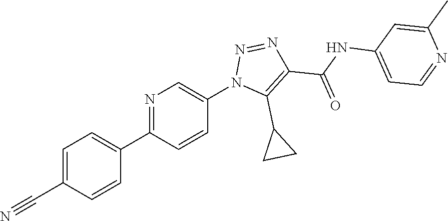 [SMILES: Cc1cc(NC(=O)c2nnn(-c3ccc(-c4ccc(C#N)cc4)nc3)c2C2CC2)ccn1] | 7-242 | 9.37 | 421.5 | 421.9 | [M + H]⁺ | 2 |
| 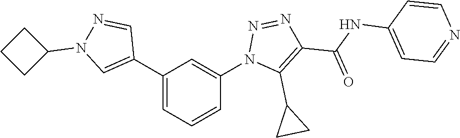 [SMILES: O=C(Nc1ccncc1)c1nnn(-c2cccc(-c3cnn(C4CCC4)c3)c2)c1C1CC1] | 7-243 | 11.46 | 425.5 | 426.2 | [M + H]⁺ | 2 |
| 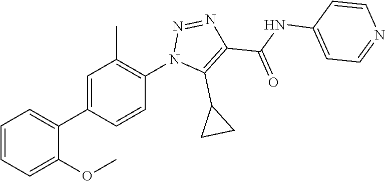 [SMILES: COc1ccccc1-c1ccc(-n2nnc(C(=O)Nc3ccncc3)c2C2CC2)c(C)c1] | 7-244 | 12.52 | 425.5 | 426.2 | [M + H]⁺ | 2 |
| 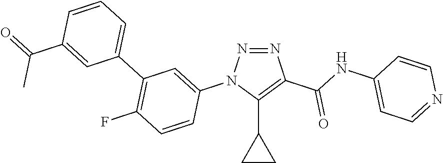 [SMILES: CC(=O)c1cccc(-c2cc(-n3nnc(C(=O)Nc4ccncc4)c3C3CC3)ccc2F)c1] | 7-245 | 11.4 | 441.5 | 442.1 | [M + H]⁺ | 2 |
| 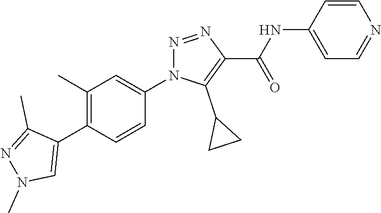 [SMILES: Cc1cc(-n2nnc(C(=O)Nc3ccncc3)c2C2CC2)ccc1-c1cn(C)nc1C] | 7-246 | 10.66 | 413.5 | 414.4 | [M + H]⁺ | 2 |
| 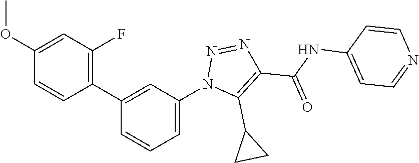 [SMILES: COc1ccc(-c2cccc(-n3nnc(C(=O)Nc4ccncc4)c3C3CC3)c2)c(F)c1] | 7-247 | 12.37 | 429.5 | 430.3 | [M + H]⁺ | 2 |
| 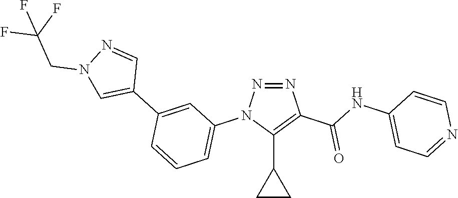 [SMILES: O=C(Nc1ccncc1)c1nnn(-c2cccc(-c3cnn(CC(F)(F)F)c3)c2)c1C1CC1] | 7-248 | 10.88 | 453.4 | 454.1 | [M + H]⁺ | 2 |
| 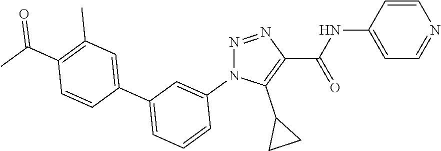 [SMILES: CC(=O)c1ccc(-c2cccc(-n3nnc(C(=O)Nc4ccncc4)c3C3CC3)c2)cc1C] | 7-249 | 11.85 | 437.5 | 438.1 | [M + H]⁺ | 2 |
| 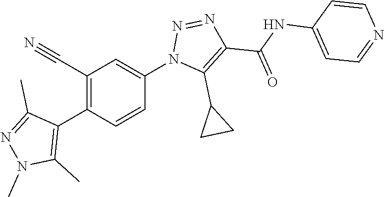 [SMILES: Cc1nn(C)c(C)c1-c1ccc(-n2nnc(C(=O)Nc3ccncc3)c2C2CC2)cc1C#N] | 7-250 | 9.85 | 438.5 | 439 | [M + H]⁺ | 2 |
| 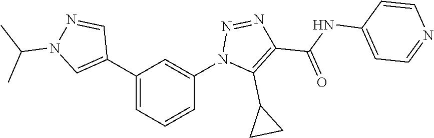 [SMILES: CC(C)n1cc(-c2cccc(-n3nnc(C(=O)Nc4ccncc4)c3C3CC3)c2)cn1] | 7-251 | 11.05 | 413.5 | 414.1 | [M + H]⁺ | 2 |
| 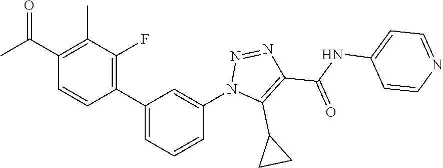 [SMILES: CC(=O)c1ccc(-c2cccc(-n3nnc(C(=O)Nc4ccncc4)c3C3CC3)c2)c(F)c1C] | 7-252 | 11.91 | 455.5 | 456.1 | [M + H]⁺ | 2 |
| 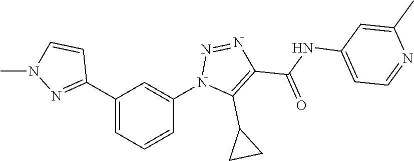 | 7-253 | 10.5 | 399.5 | 399.9 | [M + H]⁺ | 2 |
| 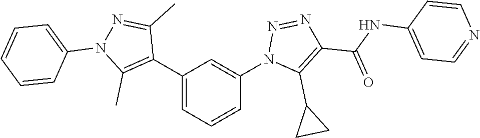 [SMILES: Cc1nn(-c2ccccc2)c(C)c1-c1cccc(-n2nnc(C(=O)Nc3ccncc3)c2C2CC2)c1] | 7-255 | 11.85 | 475.6 | 475.9 | [M + H]⁺ | 2 |
| 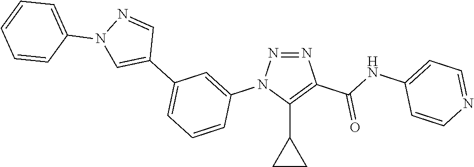 [SMILES: O=C(Nc1ccncc1)c1nnn(-c2cccc(-c3cnn(-c4ccccc4)c3)c2)c1C1CC1] | 7-256 | 11.74 | 447.5 | 448.2 | [M + H]⁺ | 2 |
| 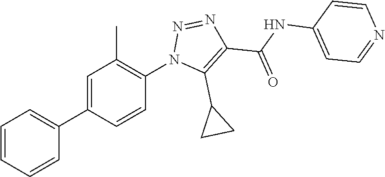 [SMILES: Cc1cc(-c2ccccc2)ccc1-n1nnc(C(=O)Nc2ccncc2)c1C1CC1] | 7-257 | 12.35 | 395.5 | 396 | [M + H]⁺ | 2 |
| 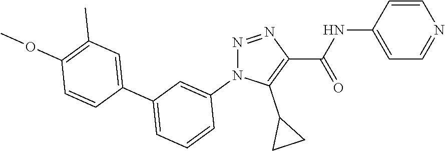 [SMILES: COc1ccc(-c2cccc(-n3nnc(C(=O)Nc4ccncc4)c3C3CC3)c2)cc1C] | 7-258 | 12.94 | 425.5 | 426.2 | [M + H]⁺ | 2 |
| 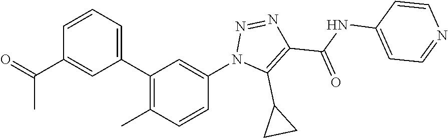 [SMILES: CC(=O)c1cccc(-c2cc(-n3nnc(C(=O)Nc4ccncc4)c3C3CC3)ccc2C)c1] | 7-259 | 11.56 | 437.5 | 438 | [M + H]⁺ | 2 |
| 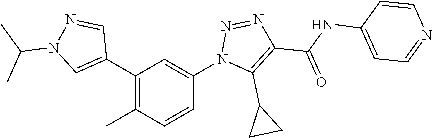 [SMILES: Cc1ccc(-n2nnc(C(=O)Nc3ccncc3)c2C2CC2)cc1-c1cnn(C(C)C)c1] | 7-260 | 11.22 | 427.5 | 428.2 | [M + H]⁺ | 2 |
| 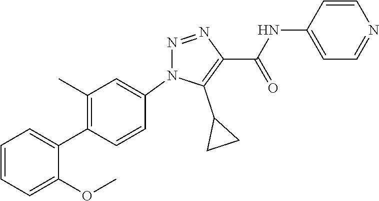 [SMILES: COc1ccccc1-c1ccc(-n2nnc(C(=O)Nc3ccncc3)c2C2CC2)cc1C] | 7-261 | 12.4 | 425.5 | 426.1 | [M + H]⁺ | 2 |
| 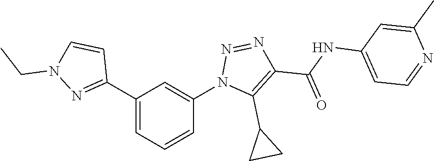 [SMILES: CCn1ccc(-c2cccc(-n3nnc(C(=O)Nc4ccnc(C)c4)c3C3CC3)c2)n1] | 7-262 | 10.93 | 413.5 | 414.2 | [M + H]⁺ | 2 |
| 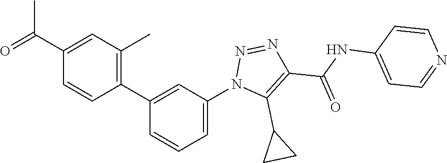 [SMILES: CC(=O)c1ccc(-c2cccc(-n3nnc(C(=O)Nc4ccncc4)c3C3CC3)c2)c(C)c1] | 7-263 | 11.64 | 437.5 | 437.9 | [M + H]⁺ | 2 |
| 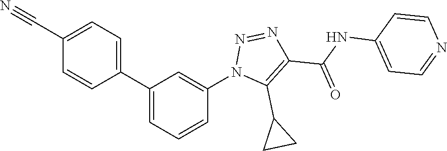 [SMILES: N#Cc1ccc(-c2cccc(-n3nnc(C(=O)Nc4ccncc4)c3C3CC3)c2)cc1] | 7-264 | 11.18 | 406.4 | 407.2 | [M + H]⁺ | 2 |
| 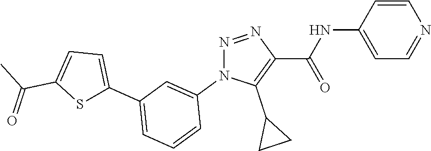 [SMILES: CC(=O)c1ccc(-c2cccc(-n3nnc(C(=O)Nc4ccncc4)c3C3CC3)c2)s1] | 7-265 | 11.18 | 429.5 | 430.2 | [M + H]⁺ | 2 |
| 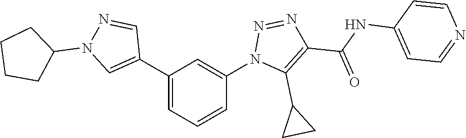 [SMILES: O=C(Nc1ccncc1)c1nnn(-c2cccc(-c3cnn(C4CCCC4)c3)c2)c1C1CC1] | 7-266 | 11.8 | 439.5 | 440.3 | [M + H]⁺ | 2 |
| 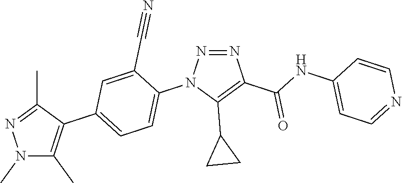 [SMILES: Cc1nn(C)c(C)c1-c1ccc(-n2nnc(C(=O)Nc3ccncc3)c2C2CC2)c(C#N)c1] | 7-267 | 9.98 | 438.5 | 439 | [M + H]⁺ | 2 |
| 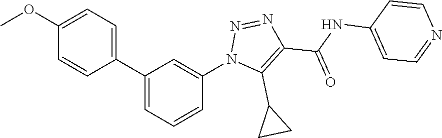 [SMILES: COc1ccc(-c2cccc(-n3nnc(C(=O)Nc4ccncc4)c3C3CC3)c2)cc1] | 7-268 | 12.17 | 411.5 | 412.1 | [M + H]⁺ | 2 |
| 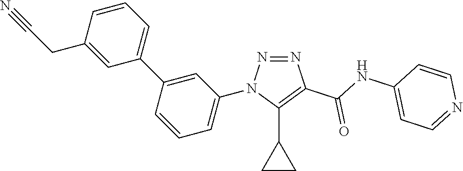 [SMILES: N#CCc1cccc(-c2cccc(-n3nnc(C(=O)Nc4ccncc4)c3C3CC3)c2)c1] | 7-269 | 11 | 420.5 | 420.7 | [M + H]⁺ | 2 |
| 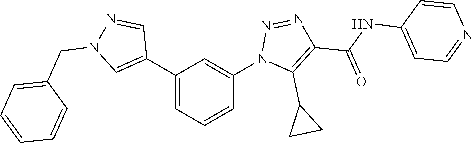 [SMILES: O=C(Nc1ccncc1)c1nnn(-c2cccc(-c3cnn(Cc4ccccc4)c3)c2)c1C1CC1] | 7-270 | 11.68 | 461.5 | 462.2 | [M + H]⁺ | 2 |
| 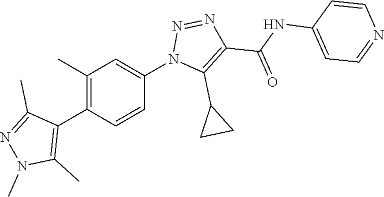 | 7-271 | 11.05 | 441.5 | 442.1 | [M + H]⁺ | 2 |
| 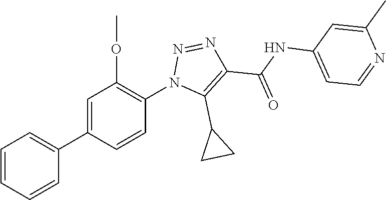 [SMILES: COc1cc(-c2ccccc2)ccc1-n1nnc(C(=O)Nc2ccnc(C)c2)c1C1CC1] | 7-272 | 11.12 | 425.5 | 425.8 | [M + H]⁺ | 2 |
| 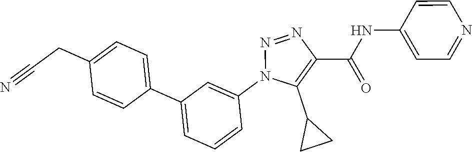 [SMILES: N#CCc1ccc(-c2cccc(-n3nnc(C(=O)Nc4ccncc4)c3C3CC3)c2)cc1] | 7-273 | 11.09 | 420.5 | 420.7 | [M + H]⁺ | 2 |
| 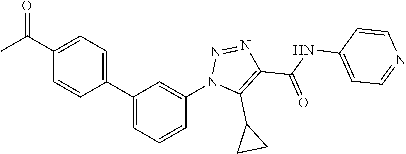 [SMILES: CC(=O)c1ccc(-c2cccc(-n3nnc(C(=O)Nc4ccncc4)c3C3CC3)c2)cc1] | 7-274 | 11.2 | 423.5 | 423.8 | [M + H]⁺ | 2 |
| 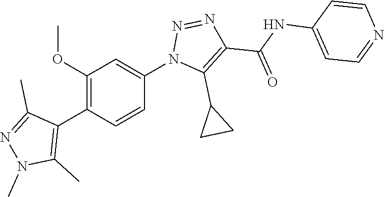 [SMILES: COc1cc(-n2nnc(C(=O)Nc3ccncc3)c2C2CC2)ccc1-c1c(C)nn(C)c1C] | 7-275 | 10.13 | 443.5 | 444 | [M + H]⁺ | 2 |
| 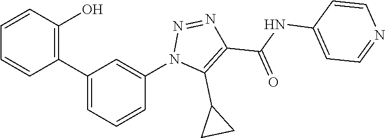 [SMILES: O=C(Nc1ccncc1)c1nnn(-c2cccc(-c3ccccc3O)c2)c1C1CC1] | 7-276 | 11.75 | 397.4 | 397.8 | [M + H]⁺ | 2 |
| 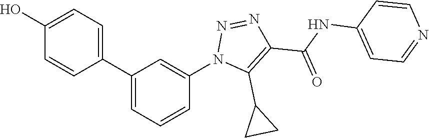 [SMILES: O=C(Nc1ccncc1)c1nnn(-c2cccc(-c3ccc(O)cc3)c2)c1C1CC1] | 7-277 | 11.25 | 397.4 | 397.8 | [M + H]⁺ | 2 |
| 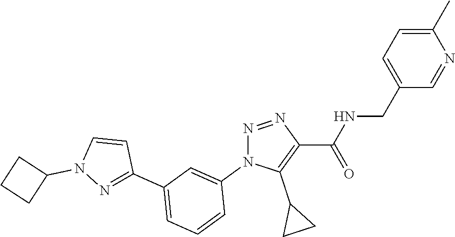 [SMILES: Cc1ccc(CNC(=O)c2nnn(-c3cccc(-c4ccn(C5CCC5)n4)c3)c2C2CC2)cn1] | 7-278 | 11.23 | 453.6 | 453.9 | [M + H]⁺ | 2 |
| 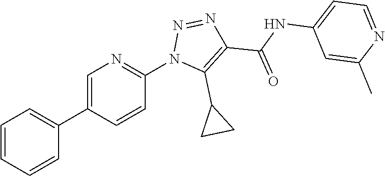 [SMILES: Cc1cc(NC(=O)c2nnn(-c3ccc(-c4ccccc4)cn3)c2C2CC2)ccn1] | 7-279 | 11.65 | 396.5 | 396.9 | [M + H]⁺ | 2 |
| 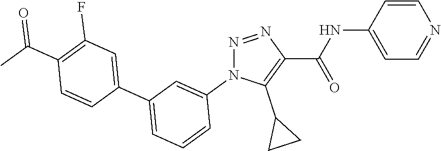 [SMILES: CC(=O)c1ccc(-c2cccc(-n3nnc(C(=O)Nc4ccncc4)c3C3CC3)c2)cc1F] | 7-280 | 11.7 | 441.5 | 442.1 | [M + H]⁺ | 2 |
| 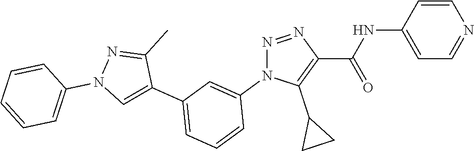 [SMILES: Cc1nn(-c2ccccc2)cc1-c1cccc(-n2nnc(C(=O)Nc3ccncc3)c2C2CC2)c1] | 7-281 | 12.09 | 461.5 | 462 | [M + H]⁺ | 2 |
| 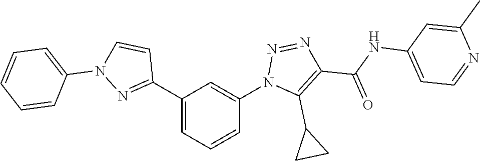 [SMILES: Cc1cc(NC(=O)c2nnn(-c3cccc(-c4ccn(-c5ccccc5)n4)c3)c2C2CC2)ccn1] | 7-282 | 12.3 | 461.5 | 461.9 | [M + H]⁺ | 2 |
| 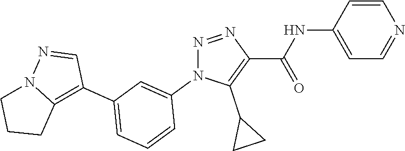 [SMILES: O=C(Nc1ccncc1)c1nnn(-c2cccc(-c3cnn4c3CCC4)c2)c1C1CC1] | 7-283 | 10.71 | 411.5 | 412 | [M + H]⁺ | 2 |
| 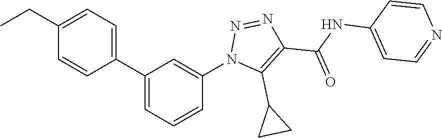 [SMILES: CCc1ccc(-c2cccc(-n3nnc(C(=O)Nc4ccncc4)c3C3CC3)c2)cc1] | 7-284 | 13.13 | 409.5 | 410.1 | [M + H]⁺ | 2 |
| 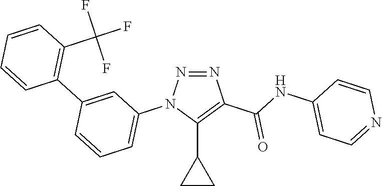 [SMILES: O=C(Nc1ccncc1)c1nnn(-c2cccc(-c3ccccc3C(F)(F)F)c2)c1C1CC1] | 7-285 | 12.32 | 449.4 | 449.9 | [M + H]⁺ | 2 |
| 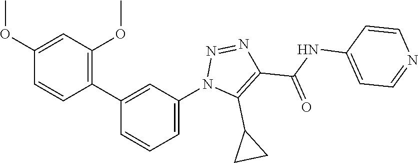 [SMILES: COc1ccc(-c2cccc(-n3nnc(C(=O)Nc4ccncc4)c3C3CC3)c2)c(OC)c1] | 7-286 | 12.3 | 441.5 | 442.3 | [M + H]⁺ | 2 |
| 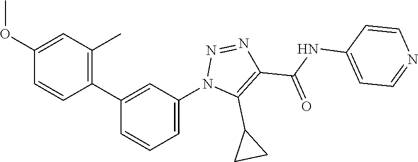 [SMILES: COc1ccc(-c2cccc(-n3nnc(C(=O)Nc4ccncc4)c3C3CC3)c2)c(C)c1] | 7-287 | 12.47 | 425.5 | 426.2 | [M + H]⁺ | 2 |
| 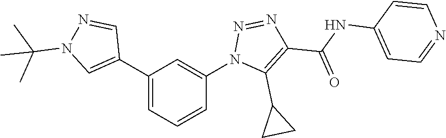 [SMILES: CC(C)(C)n1cc(-c2cccc(-n3nnc(C(=O)Nc4ccncc4)c3C3CC3)c2)cn1] | 7-288 | 11.48 | 427.5 | 428.4 | [M + H]⁺ | 2 |
| 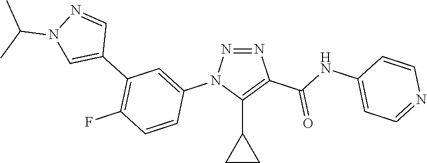 [SMILES: CC(C)n1cc(-c2cc(-n3nnc(C(=O)Nc4ccncc4)c3C3CC3)ccc2F)cn1] | 7-289 | 11.27 | 431.5 | 432.2 | [M + H]⁺ | 2 |
| 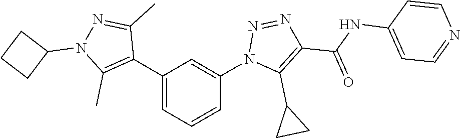 [SMILES: Cc1nn(C2CCC2)c(C)c1-c1cccc(-n2nnc(C(=O)Nc3ccncc3)c2C2CC2)c1] | 7-290 | 11.55 | 453.6 | 453.6 | [M + H]⁺ | 2 |
| 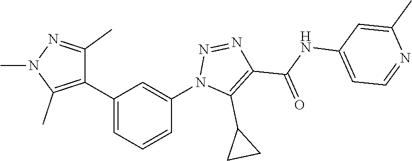 | 7-291 | 10.74 | 427.5 | 428 | [M + H]⁺ | 2 |
| 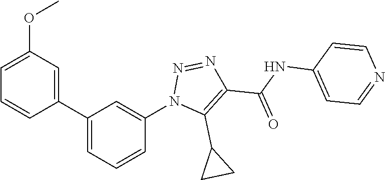 [SMILES: COc1cccc(-c2cccc(-n3nnc(C(=O)Nc4ccncc4)c3C3CC3)c2)c1] | 7-292 | 12.08 | 411.5 | 412.1 | [M + H]⁺ | 2 |
| 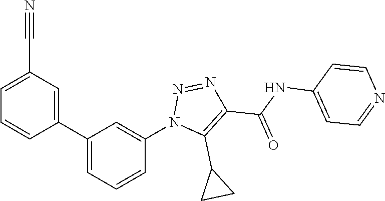 [SMILES: N#Cc1cccc(-c2cccc(-n3nnc(C(=O)Nc4ccncc4)c3C3CC3)c2)c1] | 7-293 | 11.13 | 406.4 | 407.1 | [M + H]⁺ | 2 |
| 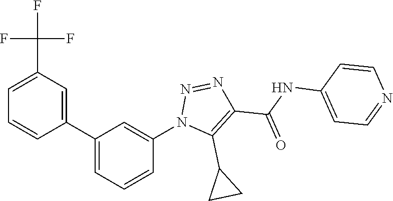 [SMILES: O=C(Nc1ccncc1)c1nnn(-c2cccc(-c3cccc(C(F)(F)F)c3)c2)c1C1CC1] | 7-294 | 12.58 | 449.4 | 449.9 | [M + H]⁺ | 2 |
| 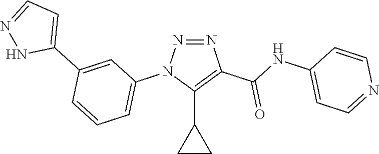 [SMILES: O=C(Nc1ccncc1)c1nnn(-c2cccc(-c3ccn[nH]3)c2)c1C1CC1] | 7-295 | 8.43 | 371.4 | 371.8 | [M + H]⁺ | 2 |
| 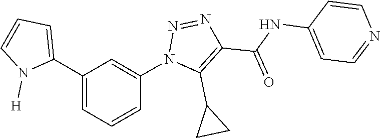 [SMILES: O=C(Nc1ccncc1)c1nnn(-c2cccc(-c3ccc[nH]3)c2)c1C1CC1] | 7-296 | 10.99 | 370.4 | 370.9 | [M + H]⁺ | 2 |
| 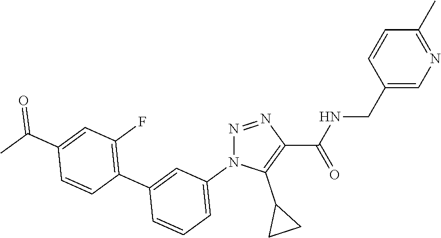 [SMILES: CC(=O)c1ccc(-c2cccc(-n3nnc(C(=O)NCc4ccc(C)nc4)c3C3CC3)c2)c(F)c1] | 7-297 | 11.05 | 469.5 | 470 | [M + H]⁺ | 2 |
| 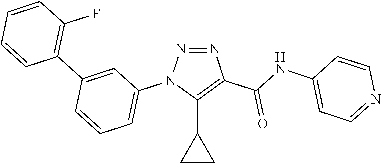 [SMILES: O=C(Nc1ccncc1)c1nnn(-c2cccc(-c3ccccc3F)c2)c1C1CC1] | 7-298 | 12.29 | 399.4 | 400.2 | [M + H]⁺ | 2 |
| 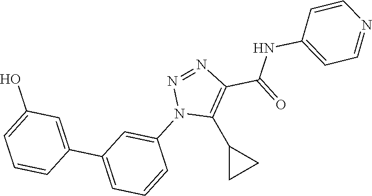 [SMILES: O=C(Nc1ccncc1)c1nnn(-c2cccc(-c3cccc(O)c3)c2)c1C1CC1] | 7-299 | 11.12 | 397.4 | 397.9 | [M + H]⁺ | 2 |
| 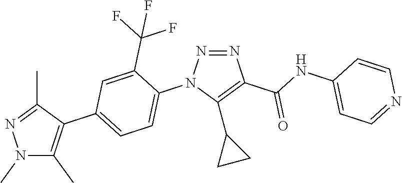 [SMILES: Cc1nn(C)c(C)c1-c1ccc(-n2nnc(C(=O)Nc3ccncc3)c2C2CC2)c(C(F)(F)F)c1] | 7-300 | 11.24 | 481.5 | 482.1 | [M + H]⁺ | 2 |
| 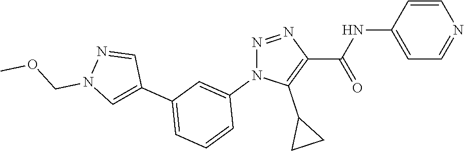 [SMILES: COCn1cc(-c2cccc(-n3nnc(C(=O)Nc4ccncc4)c3C3CC3)c2)cn1] | 7-301 | 10.27 | 415.5 | 416.4 | [M + H]⁺ | 2 |
| 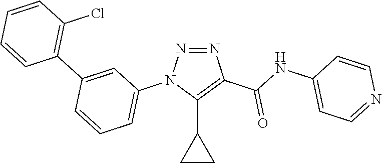 [SMILES: O=C(Nc1ccncc1)c1nnn(-c2cccc(-c3ccccc3Cl)c2)c1C1CC1] | 7-302 | 12.49 | 415.9 | 415.9 | [M + H]⁺ | 2 |
| 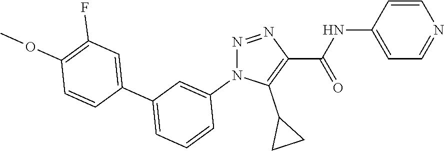 [SMILES: COc1ccc(-c2cccc(-n3nnc(C(=O)Nc4ccncc4)c3C3CC3)c2)cc1F] | 7-303 | 12 | 429.5 | 430.2 | [M + H]⁺ | 2 |
| 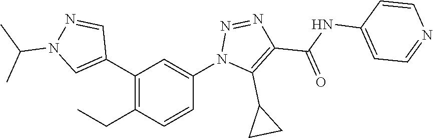 [SMILES: CCc1ccc(-n2nnc(C(=O)Nc3ccncc3)c2C2CC2)cc1-c1cnn(C(C)C)c1] | 7-304 | 11.73 | 441.5 | 442.1 | [M + H]⁺ | 2 |
| 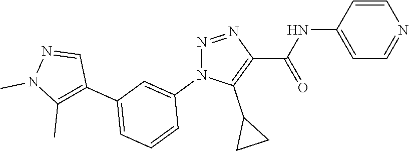 [SMILES: Cc1c(-c2cccc(-n3nnc(C(=O)Nc4ccncc4)c3C3CC3)c2)cnn1C] | 7-305 | 10.55 | 399.5 | 400.2 | [M + H]⁺ | 2 |
| 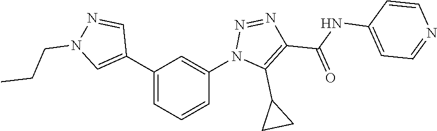 [SMILES: CCCn1cc(-c2cccc(-n3nnc(C(=O)Nc4ccncc4)c3C3CC3)c2)cn1] | 7-306 | 11.14 | 413.5 | 414.2 | [M + H]⁺ | 2 |
| 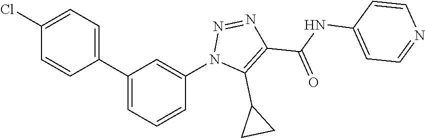 [SMILES: O=C(Nc1ccncc1)c1nnn(-c2cccc(-c3ccc(Cl)cc3)c2)c1C1CC1] | 7-307 | 12.65 | 415.9 | 416.2 | [M + H]⁺ | 2 |
| 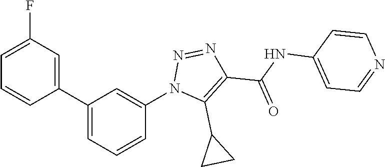 [SMILES: O=C(Nc1ccncc1)c1nnn(-c2cccc(-c3cccc(F)c3)c2)c1C1CC1] | 7-308 | 12.22 | 399.4 | 400.1 | [M + H]⁺ | 2 |
| 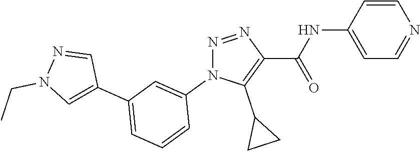 [SMILES: CCn1cc(-c2cccc(-n3nnc(C(=O)Nc4ccncc4)c3C3CC3)c2)cn1] | 7-309 | 10.6 | 399.5 | 400.2 | [M + H]⁺ | 2 |
| 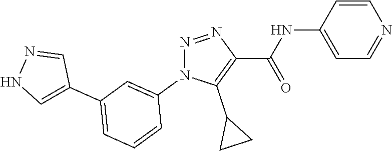 [SMILES: O=C(Nc1ccncc1)c1nnn(-c2cccc(-c3cn[nH]c3)c2)c1C1CC1] | 7-310 | 9.89 | 371.4 | 371.9 | [M + H]⁺ | 2 |
| 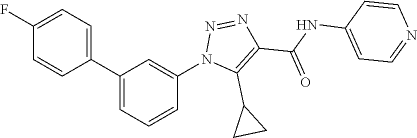 [SMILES: O=C(Nc1ccncc1)c1nnn(-c2cccc(-c3ccc(F)cc3)c2)c1C1CC1] | 7-311 | 12.15 | 399.4 | 399.9 | [M + H]⁺ | 2 |
| 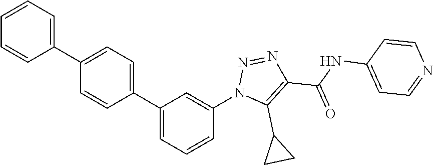 [SMILES: O=C(Nc1ccncc1)c1nnn(-c2cccc(-c3ccc(-c4ccccc4)cc3)c2)c1C1CC1] | 7-312 | 13.6 | 457.5 | 458 | [M + H]⁺ | 2 |
| 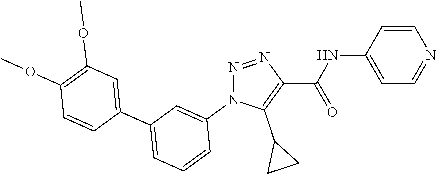 [SMILES: COc1ccc(-c2cccc(-n3nnc(C(=O)Nc4ccncc4)c3C3CC3)c2)cc1OC] | 7-313 | 11.54 | 441.5 | 442.2 | [M + H]⁺ | 2 |
| 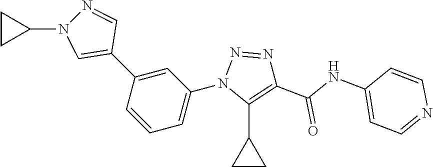 [SMILES: O=C(Nc1ccncc1)c1nnn(-c2cccc(-c3cnn(C4CC4)c3)c2)c1C1CC1] | 7-314 | 10.93 | 411.5 | 412 | [M + H]⁺ | 2 |
| 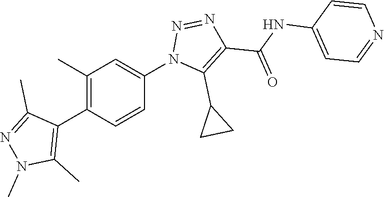 [SMILES: Cc1cc(-n2nnc(C(=O)Nc3ccncc3)c2C2CC2)ccc1-c1c(C)nn(C)c1C] | 7-315 | 10.67 | 427.5 | 428.2 | [M + H]⁺ | 2 |
| 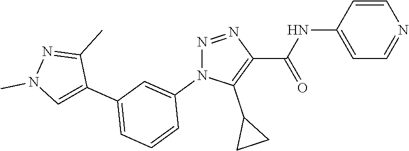 [SMILES: Cc1nn(C)cc1-c1cccc(-n2nnc(C(=O)Nc3ccncc3)c2C2CC2)c1] | 7-316 | 10.6 | 399.5 | 400.1 | [M + H]⁺ | 2 |
| 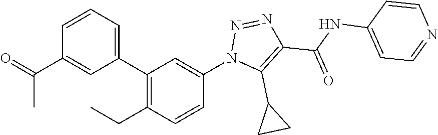 [SMILES: CCc1ccc(-n2nnc(C(=O)Nc3ccncc3)c2C2CC2)cc1-c1cccc(C(C)=O)c1] | 7-317 | 12.04 | 451.5 | 451.9 | [M + H]⁺ | 2 |
| 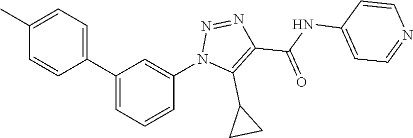 [SMILES: Cc1ccc(-c2cccc(-n3nnc(C(=O)Nc4ccncc4)c3C3CC3)c2)cc1] | 7-318 | 12.75 | 395.5 | 396.1 | [M + H]⁺ | 2 |
| 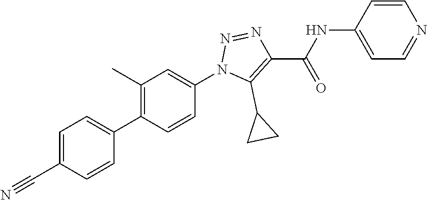 [SMILES: Cc1cc(-n2nnc(C(=O)Nc3ccncc3)c2C2CC2)ccc1-c1ccc(C#N)cc1] | 7-319 | 11.53 | 420.5 | 421 | [M + H]⁺ | 2 |
| 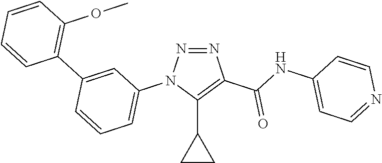 [SMILES: COc1ccccc1-c1cccc(-n2nnc(C(=O)Nc3ccncc3)c2C2CC2)c1] | 7-320 | 12.34 | 411.5 | 412.1 | [M + H]⁺ | 2 |
| 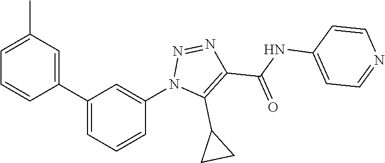 [SMILES: Cc1cccc(-c2cccc(-n3nnc(C(=O)Nc4ccncc4)c3C3CC3)c2)c1] | 7-321 | 12.79 | 395.5 | 396.1 | [M + H]⁺ | 2 |
| 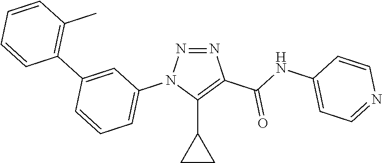 [SMILES: Cc1ccccc1-c1cccc(-n2nnc(C(=O)Nc3ccncc3)c2C2CC2)c1] | 7-322 | 12.64 | 395.5 | 396.1 | [M + H]⁺ | 2 |
| 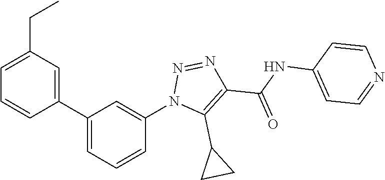 [SMILES: CCc1cccc(-c2cccc(-n3nnc(C(=O)Nc4ccncc4)c3C3CC3)c2)c1] | 7-323 | 12.99 | 409.5 | 410 | [M + H]⁺ | 2 |
| 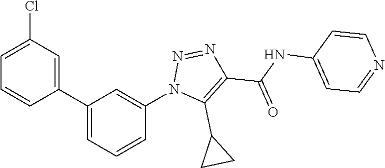 [SMILES: O=C(Nc1ccncc1)c1nnn(-c2cccc(-c3cccc(Cl)c3)c2)c1C1CC1] | 7-324 | 12.57 | 415.9 | 416.1 | [M + H]⁺ | 2 |
| 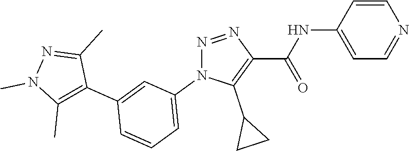 [SMILES: Cc1nn(C)c(C)c1-c1cccc(-n2nnc(C(=O)Nc3ccncc3)c2C2CC2)c1] | 7-325 | 10.62 | 413.5 | 414.1 | [M + H]⁺ | 2 |
| 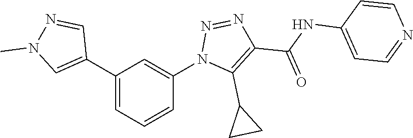 [SMILES: Cn1cc(-c2cccc(-n3nnc(C(=O)Nc4ccncc4)c3C3CC3)c2)cn1] | 7-326 | 10.28 | 385.4 | 386.1 | [M + H]⁺ | 2 |
| 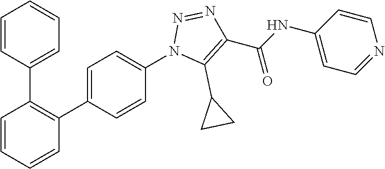 [SMILES: O=C(Nc1ccncc1)c1nnn(-c2ccc(-c3ccccc3-c3ccccc3)cc2)c1C1CC1] | 7-327 | 13.34 | 457.5 | 458.3 | [M + H]⁺ | 2 |
| 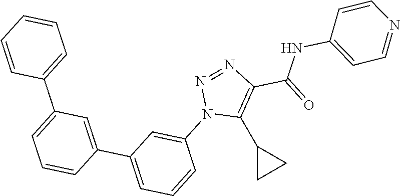 [SMILES: O=C(Nc1ccncc1)c1nnn(-c2cccc(-c3cccc(-c4ccccc4)c3)c2)c1C1CC1] | 7-328 | 13.58 | 457.5 | 458 | [M + H]⁺ | 2 |
| 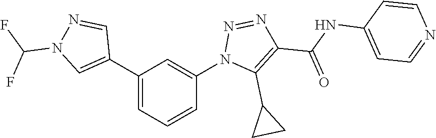 [SMILES: O=C(Nc1ccncc1)c1nnn(-c2cccc(-c3cnn(C(F)F)c3)c2)c1C1CC1] | 7-329 | 10.4 | 421.4 | 422.1 | [M + H]⁺ | 2 |
| 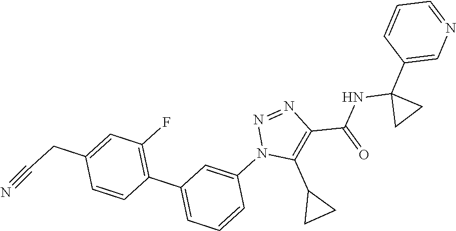 [SMILES: N#CCc1ccc(-c2cccc(-n3nnc(C(=O)NC4(c5cccnc5)CC4)c3C3CC3)c2)c(F)c1] | 7-330 | 10.81 | 478.5 | 478.8 | [M + H]⁺ | 2 |
| 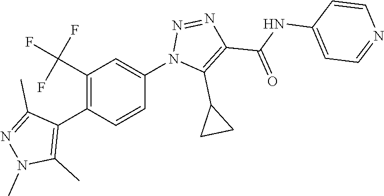 [SMILES: Cc1nn(C)c(C)c1-c1ccc(-n2nnc(C(=O)Nc3ccncc3)c2C2CC2)cc1C(F)(F)F] | 7-331 | 11.06 | 481.5 | 482.1 | [M + H]⁺ | 2 |
| 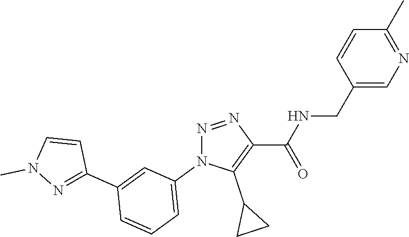 | 7-332 | 9.69 | 413.5 | 414 | [M + H]⁺ | 2 |
| 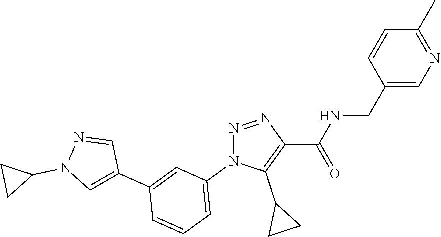 [SMILES: Cc1ccc(CNC(=O)c2nnn(-c3cccc(-c4cnn(C5CC5)c4)c3)c2C2CC2)cn1] | 7-333 | 10.17 | 439.5 | 440.1 | [M + H]⁺ | 2 |
| 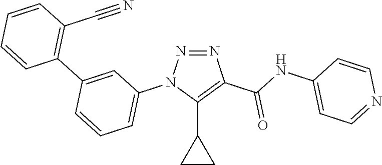 [SMILES: N#Cc1ccccc1-c1cccc(-n2nnc(C(=O)Nc3ccncc3)c2C2CC2)c1] | 7-334 | 11.11 | 406.4 | 406.9 | [M + H]⁺ | 2 |
| 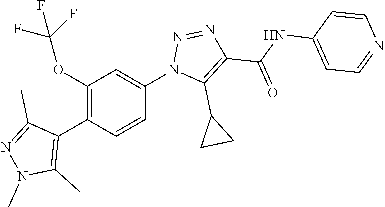 [SMILES: Cc1nn(C)c(C)c1-c1ccc(-n2nnc(C(=O)Nc3ccncc3)c2C2CC2)cc1OC(F)(F)F] | 7-335 | 11.28 | 497.5 | 497.9 | [M + H]⁺ | 2 |

### Example 8

### Synthesis of Compound 8-2 and Other Representative Compounds

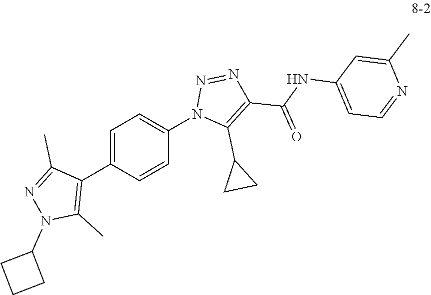 [SMILES: Cc1cc(NC(=O)c2nnn(-c3ccc(-c4c(C)nn(C5CCC5)c4C)cc3)c2C2CC2)ccn1]

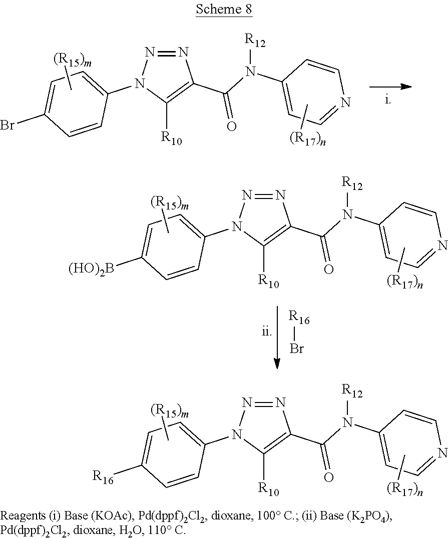

### Step 8-1. Synthesis of (4-(5-cyclopropyl-4-((2-methylpyridin-4-yl)carbamoyl)-1H-1,2,3-triazol-1-yl)phenyl)boronic acid (INT 8A)

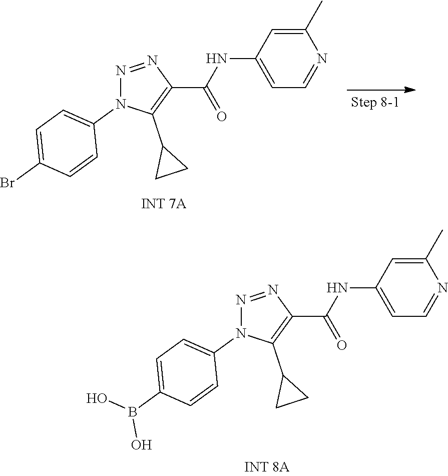 [SMILES: Cc1cc(NC(=O)c2nnn(-c3ccc(Br)cc3)c2C2CC2)ccn1 ; Cc1cc(NC(=O)c2nnn(-c3ccc(B(O)O)cc3)c2C2CC2)ccn1]

Dioxane (4 mL) was added to a reaction vial containing INT 7A (400 mg, 1.04 mmol), 4,4,4′,4′,5,5,5′,5′-octamethyl-2,2′-bi(1,3,2-dioxaborolane) (528 mg, 2.1 mmol), Pd(dppfCl₂·CH₂Cl₂ (85 mg, 0.1 mmol) and KOAc (306.5 mg, 3.12 mmol). The reaction mixture was deoxygenated using N₂, heated at 100° C. for 2 h, and then concentrated. The resulting residue was dissolved in 1 N aqueous NaOH (20 mL) and filtered. The filtrate was washed with DCM (2×). The organic layers were back-extracted with 1 N NaOH and water. The combined aqueous layers were acidified to pH 6.5-7 and filtered to collect the resulting solid, which was then washed with H₂O and dried under vacuum at 60° C. for 2 h to provide 320 mg (85%) of (4-(5-cyclopropyl-4-((2-methylpyridin-4-yl)carbamoyl)-1H-1,2,3-triazol-1-yl)phenyl)boronic acid (INT 8A) as a brown solid. LCMS-ESI (m/z) calculated for C₁₈H₁₈BN₅O₃: 363.1, found 364 [M+H]⁺, t_{R}=5.34 min (Method 3)

### Step 8-2. Synthesis of 1-(4-(1-cyclobutyl-3,5-dimethyl-1H-pyrazol-4-yl)phenyl)-5-cyclopropyl-N-(2-methylpyridin-4-yl)-1H-1,2,3-triazole-4-carboxamide (Compound 8-2)

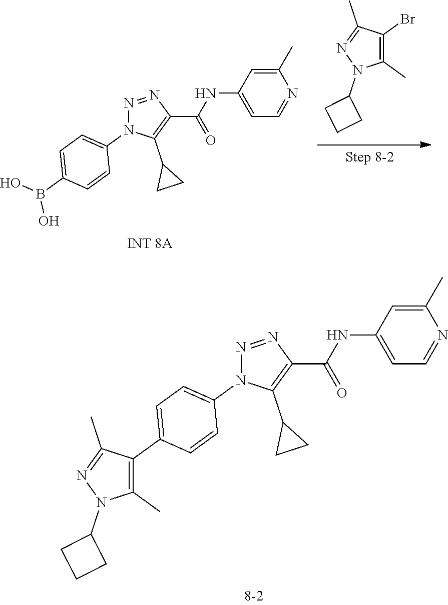

Into a sealable tube containing INT 8A (30 mg, 0.08 mmol), 4-bromo-1-cyclobutyl-3,5-dimethyl-1H-pyrazole (34 mg, 0.15 mmol), Pd(dppf)Cl₂ (6 mg, 0.008 mmol), and K₂PO₄ (52.6 mg, 0.25 mmol) were added dioxane (3 mL) and H₂O (1 mL). The mixture was stirred to dissolve the components and then purged with N₂, sealed, and heated at 110° C. for 3 hr. Additional 4-bromo-1-cyclobutyl-3,5-dimethyl-1H-pyrazole (17 mg, 0.007 mmol) and Pd(dppf)Cl₂ (5 mg, 0.007 mmol) were added, and the reaction mixture was purged with N₂, heated, and stirred for another 2 hr. The reaction mixture was diluted with DCM, washed with NaHCO₃ and brine, dried (Na₂SO₄), filtered, concentrated and purified by SiO₂ chromatography (EA) and reverse phase-SiO₂ chromatography (MeOH/H₂O) to provide 13.1 mg (33.9%) of 1-(4-(1-cyclobutyl-3,5-dimethyl-1H-pyrazol-4-yl)phenyl)-5-cyclopropyl-N-(2-methylpyridin-4-yl)-1H-1,2,3-triazole-4-carboxamide (Compound 8-2). LCMS-ESI (m/z) calculated for C₂₇H₂₉N₇O: 467.6; found 468.0 [M+H]⁺, t_{R}=11.52 min (Method 2). ¹H NMR (400 MHz, CDCl₃) δ=9.21 (s, 1H), 8.31 (s, 1H), 7.44-7.50 (m, 3H), 7.30-7.33 (m, 3H), 4.55 (m, 1H), 2.61 (m, 2H), 2.49 (s, 3H), 2.32 (m, 2H), 2.20 (s, 3H), 2.15 (s, 3H), 1.93 (m, 1H), 1.88-1.90 (m, 2H), 1.21 (m, 2H), 0.98 (m, 2H).

The compounds listed in Table 8 were made using the procedures of Scheme 8.

TABLE 8

| | | Purity | | | | |
|---|---|---|---|---|---|---|
| | Cpd | RT | | Observed | | Purity |
| Structure | No. | (min) | MW | m/z | Ion | Method |
| 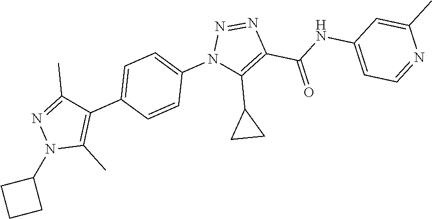 [SMILES: Cc1cc(NC(=O)c2nnn(-c3ccc(-c4c(C)nn(C5CCC5)c4C)cc3)c2C2CC2)ccn1] | 8-1 | 11.65 | 449.5 | 449.9 | [M + H]⁺ | 2 |
| 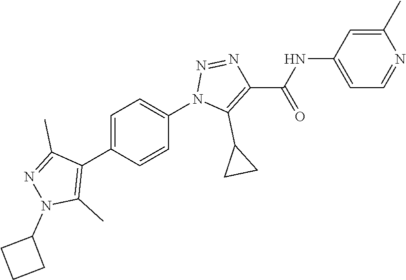 [SMILES: Cc1cc(NC(=O)c2nnn(-c3ccc(-c4c(C)nn(C5CCC5)c4C)cc3)c2C2CC2)ccn1] | 8-2 | 11.92 | 467.6 | 468 | [M + H]⁺ | 2 |
| 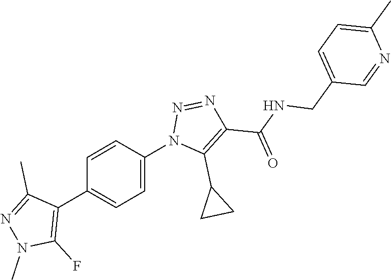 [SMILES: Cc1ccc(CNC(=O)c2nnn(-c3ccc(-c4c(C)nn(C)c4F)cc3)c2C2CC2)cn1] | 8-3 | 10.62 | 445.5 | 446 | [M + H]⁺ | 2 |
| 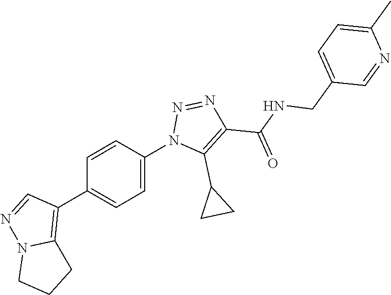 [SMILES: Cc1ccc(CNC(=O)c2nnn(-c3ccc(-c4cnn5c4CCC5)cc3)c2C2CC2)cn1] | 8-4 | 10.08 | 439.5 | 440 | [M + H]⁺ | 2 |

### Example 9

### Synthesis of Compound 9-1 and Other Representative Compounds

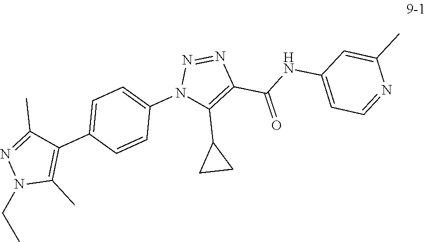 [SMILES: CCn1nc(C)c(-c2ccc(-n3nnc(C(=O)Nc4ccnc(C)c4)c3C3CC3)cc2)c1C]

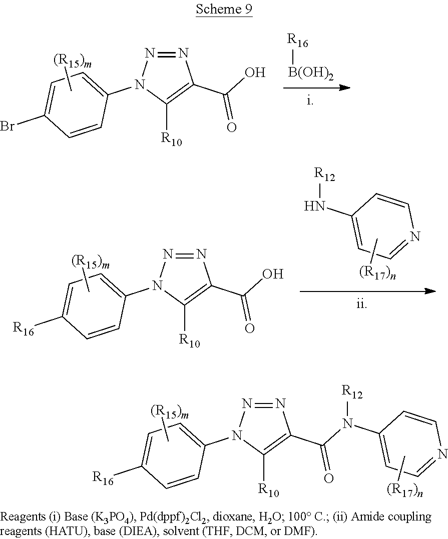

### Step 9-1. Synthesis of 5-cyclopropyl-1-(4-(1-ethyl-3,5-dimethyl-1H-pyrazol-4-yl)phenyl)-1H-1,2,3-triazole-4-carboxylic acid (INT 9A)

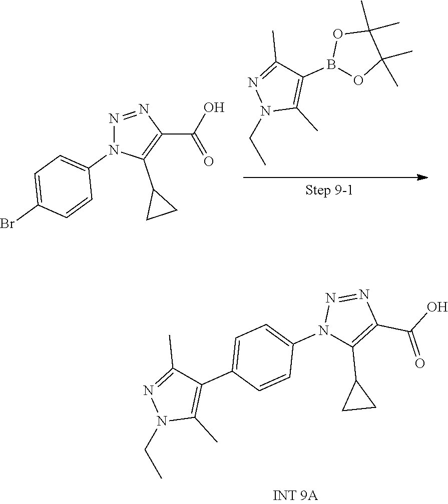 [SMILES: O=C(O)c1nnn(-c2ccc(Br)cc2)c1C1CC1 ; CCn1nc(C)c(B2OC(C)(C)C(C)(C)O2)c1C ; CCn1nc(C)c(-c2ccc(-n3nnc(C(=O)O)c3C3CC3)cc2)c1C]

To a vial containing 1-(4-bromophenyl)-5-cyclopropyl-1H-1,2,3-triazole-4-carboxylic acid (450 mg, 1.46 mmol) were added 1-ethyl-3,5-dimethyl-4-(4,4,5,5-tetramethyl-1,3,2-dioxaborolan-2-yl)-1H-pyrazole (365.3 mg, 1.46 mmol), Pd(dppf)Cl₂·CH₂Cl₂ (119.3 mg, 0.15 mmol), K₃PO₄ (930 mg, 4.38 mmol), dioxane (4 mL) and H₂O (2 mL). After degassing with N₂, the reaction mixture was heated to 100° C. and stirred for 2 h. The reaction mixture was cooled to room temperature and directly purified by RP—SiO₂ chromatography (MeOH/H₂O) to afford 317 mg, (62%) of 5-cyclopropyl-1-(4-(1-ethyl-3,5-dimethyl-1H-pyrazol-4-yl)phenyl)-1H-1,2,3-triazole-4-carboxylic acid (INT 9A) as a light brown solid. LCMS-ESI (m/z) calculated for C₁₉H₂₁N₅O₂: 351.4; found 352 [M+H]⁺, t_{R}=5.71 min (Method 3). ¹H NMR (400 MHz, DMSO-D₆) δ 13.08 (bs, 1H), 8.07 (d, J=8.0 Hz, 2H), 7.45 (d, J=8.0 Hz, 2H), 4.09 (m, 2H), 2.30 (s, 3H), 2.20 (s, 3H), 2.09 (m, 1H), 1.35 (t, J=8 Hz, 3H), 0.96 (m, 2H), 0.72 (m, 2H).

### Step 9-2. Synthesis of 5-cyclopropyl-1-(4-(1-ethyl-3,5-dimethyl-1H-pyrazol-4-yl)phenyl)-N-(2-methylpyridin-4-yl)-1H-1,2,3-triazole-4-carboxamide (Compound 9-1)

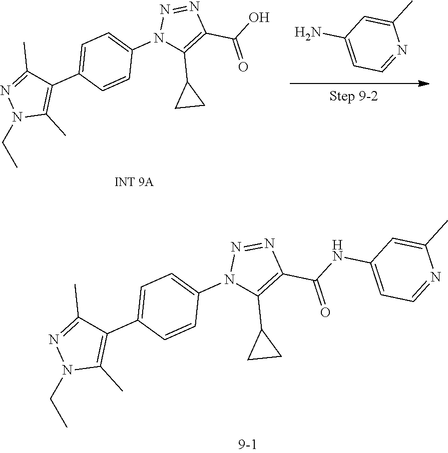

To a stirring solution of INT 9A (30 mg, 0.085 mmol) in DMF (0.8 mL) was added HATU (35.7 mg, 0.094 mmol). After stirring for 15 min, 2-methylpyridin-4-amine (10.2 mg, 0.094 mmol) and DIEA (33 mg, 0.26 mmol) were added. After stirring for 4 h, the reaction mixture diluted with EA, washed with H₂O, NaHCO₃ and brine, then dried (Na₂SO₄), filtered, concentrated, and purified by SiO₂ chromatography (EA/hexane and MeOH/DCM) to afford 13.4 mg (35.5%) of 5-cyclopropyl-1-(4-(1-ethyl-3,5-dimethyl-1H-pyrazol-4-yl)phenyl)-N-(2-methylpyridin-4-yl)-1H-1,2,3-triazole-4-carboxamide (Compound 9-1). LCMS-ESI (m/z) calculated for C₂₅H₂₇N₇O: 441.2; found 442.1 [M+H]⁺, t_{R}=10.56 min (Method 2). ¹H NMR (400 MHz, CDCl₃) δ 9.54 (s, 1H), 8.48 (d, J=8.0 Hz, 1H), 7.77 (s, 1H), 7.66 (m, 3H), 7.50 (d, J=8.0 Hz, 2H), 4.60 (m, 2H), 2.72 (s, 3H), 2.34 (s, 6H), 2.09 (m, 1H), 1.45 (t, J=8 Hz, 3H), 01.29 (m, 2H), 1.12 (m, 2H).

The compounds listed in Table 9 wase made using the procedures of Scheme 9:

TABLE 9

| | | Purity | | | | |
|---|---|---|---|---|---|---|
| | Cpd | RT | | Observed | | Purity |
| Structure | No. | (min) | MW | m/z | Ion | Method |
| 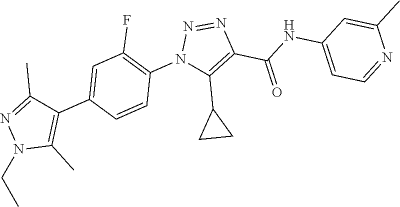 [SMILES: CCn1nc(C)c(-c2ccc(-n3nnc(C(=O)Nc4ccnc(C)c4)c3C3CC3)c(F)c2)c1C] | 9-1 | 10.8 | 441.5 | 442.1 | [M + H]⁺ | 2 |
| 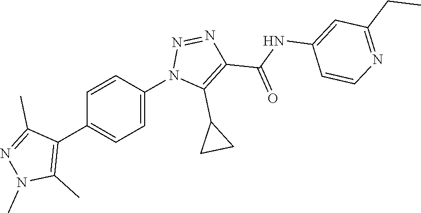 [SMILES: CCc1cc(NC(=O)c2nnn(-c3ccc(-c4c(C)nn(C)c4C)cc3)c2C2CC2)ccn1] | 9-2 | 11.24 | 441.5 | 442.2 | [M + H]⁺ | 2 |
| 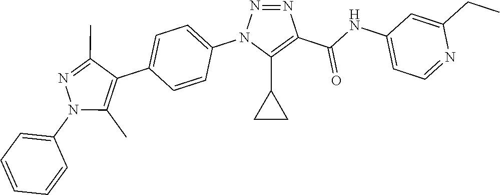 [SMILES: CCc1cc(NC(=O)c2nnn(-c3ccc(-c4c(C)nn(-c5ccccc5)c4C)cc3)c2C2CC2)ccn1] | 9-3 | 12.41 | 503.6 | 503.7 | [M + H]⁺ | 2 |
| 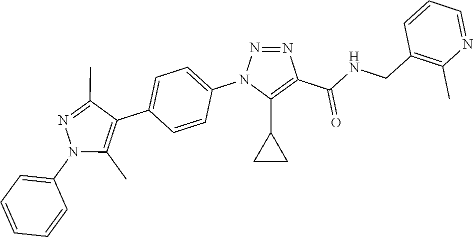 [SMILES: Cc1ncccc1CNC(=O)c1nnn(-c2ccc(-c3c(C)nn(-c4ccccc4)c3C)cc2)c1C1CC1] | 9-4 | 11.39 | 503.6 | 503.7 | [M + H]⁺ | 2 |
| 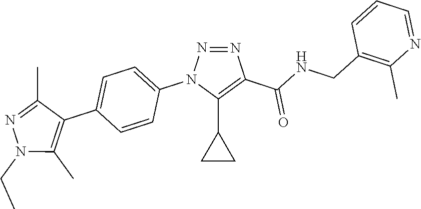 [SMILES: CCn1nc(C)c(-c2ccc(-n3nnc(C(=O)NCc4cccnc4C)c3C3CC3)cc2)c1C] | 9-5 | 10.08 | 455.6 | 455.9 | [M + H]⁺ | 2 |
| 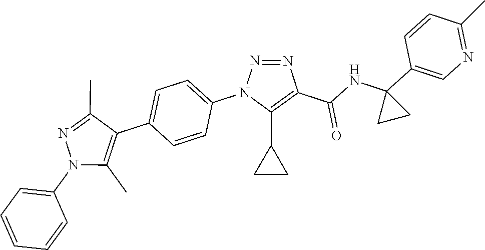 [SMILES: Cc1ccc(C2(NC(=O)c3nnn(-c4ccc(-c5c(C)nn(-c6ccccc6)c5C)cc4)c3C3CC3)CC2)cn1] | 9-6 | 11.72 | 529.6 | 529.9 | [M + H]⁺ | 2 |
| 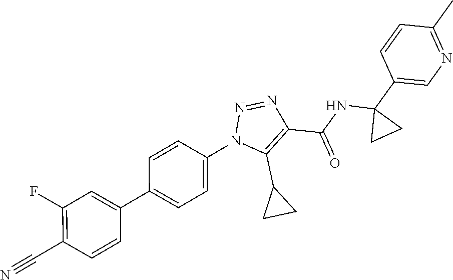 [SMILES: Cc1ccc(C2(NC(=O)c3nnn(-c4ccc(-c5ccc(C#N)c(F)c5)cc4)c3C3CC3)CC2)cn1] | 9-7 | 11.03 | 478.5 | 478.6 | [M + H]⁺ | 2 |
| 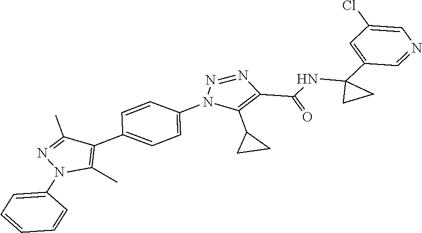 [SMILES: Cc1nn(-c2ccccc2)c(C)c1-c1ccc(-n2nnc(C(=O)NC3(c4cncc(Cl)c4)CC3)c2C2CC2)cc1] | 9-8 | 13 | 550.1 | 552.3 | [M + H]⁺ | 2 |
| 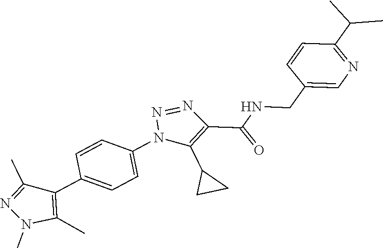 [SMILES: Cc1nn(C)c(C)c1-c1ccc(-n2nnc(C(=O)NCc3ccc(C(C)C)nc3)c2C2CC2)cc1] | 9-9 | 10.22 | 469.6 | 470 | [M + H]⁺ | 2 |
| 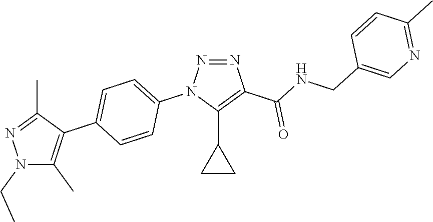 [SMILES: CCn1nc(C)c(-c2ccc(-n3nnc(C(=O)NCc4ccc(C)nc4)c3C3CC3)cc2)c1C] | 9-10 | 9.95 | 455.6 | 456.2 | [M + H]⁺ | 2 |
| 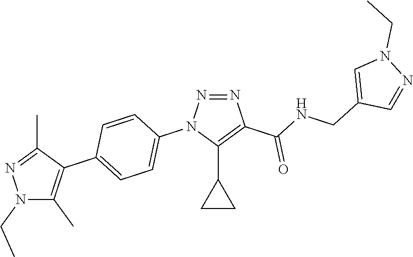 [SMILES: CCn1cc(CNC(=O)c2nnn(-c3ccc(-c4c(C)nn(CC)c4C)cc3)c2C2CC2)cn1] | 9-11 | 11.47 | 458.6 | 459.1 | [M + H]⁺ | 2 |
| 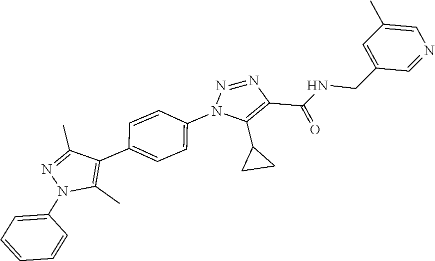 [SMILES: Cc1cncc(CNC(=O)c2nnn(-c3ccc(-c4c(C)nn(-c5ccccc5)c4C)cc3)c2C2CC2)c1] | 9-12 | 11.51 | 503.6 | 503.7 | [M + H]⁺ | 2 |
| 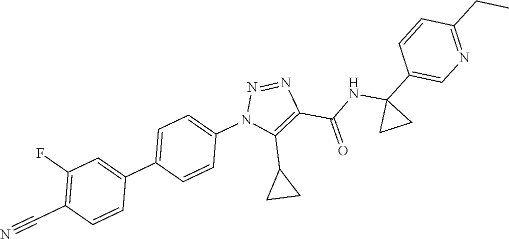 [SMILES: CCc1ccc(C2(NC(=O)c3nnn(-c4ccc(-c5ccc(C#N)c(F)c5)cc4)c3C3CC3)CC2)cn1] | 9-13 | 11.15 | 492.6 | 492.9 | [M + H]⁺ | 2 |
| 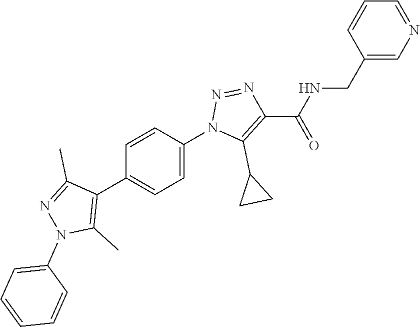 [SMILES: Cc1nn(-c2ccccc2)c(C)c1-c1ccc(-n2nnc(C(=O)NCc3cccnc3)c2C2CC2)cc1] | 9-14 | 11.36 | 489.6 | 490 | [M + H]⁺ | 2 |
| 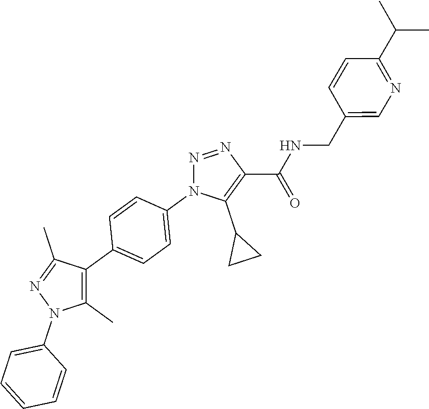 [SMILES: Cc1nn(-c2ccccc2)c(C)c1-c1ccc(-n2nnc(C(=O)NCc3ccc(C(C)C)nc3)c2C2CC2)cc1] | 9-15 | 11.71 | 531.7 | 531.9 | [M + H]⁺ | 2 |
| 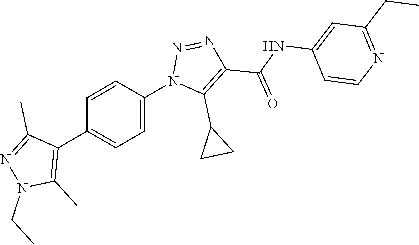 [SMILES: CCc1cc(NC(=O)c2nnn(-c3ccc(-c4c(C)nn(CC)c4C)cc3)c2C2CC2)ccn1] | 9-16 | 11.35 | 455.6 | 456.1 | [M + H]⁺ | 2 |
| 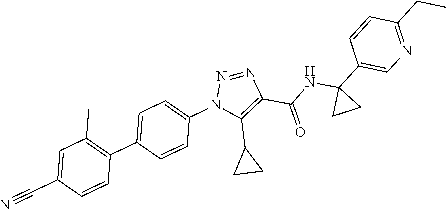 [SMILES: CCc1ccc(C2(NC(=O)c3nnn(-c4ccc(-c5ccc(C#N)cc5C)cc4)c3C3CC3)CC2)cn1] | 9-17 | 11.4 | 488.6 | 488.8 | [M + H]⁺ | 2 |
| 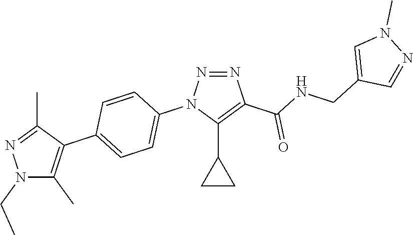 [SMILES: CCn1nc(C)c(-c2ccc(-n3nnc(C(=O)NCc4cnn(C)c4)c3C3CC3)cc2)c1C] | 9-18 | 11.14 | 444.5 | 444.9 | [M + H]⁺ | 2 |
| 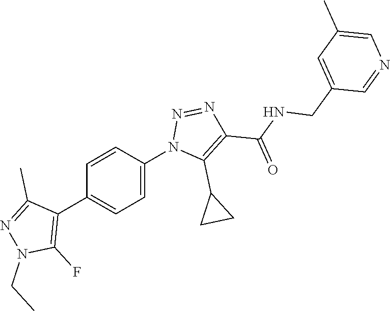 | 9-19 | 10.18 | 455.6 | 456.1 | [M + H]⁺ | 2 |
| 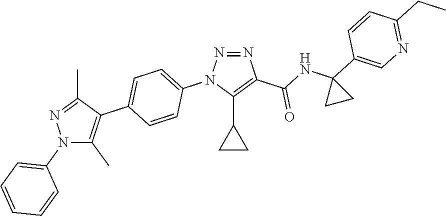 [SMILES: CCc1ccc(C2(NC(=O)c3nnn(-c4ccc(-c5c(C)nn(-c6ccccc6)c5C)cc4)c3C3CC3)CC2)cn1] | 9-20 | 11.86 | 543.7 | 543.8 | [M + H]⁺ | 2 |
| 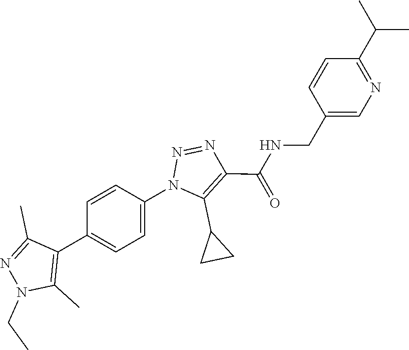 [SMILES: CCn1nc(C)c(-c2ccc(-n3nnc(C(=O)NCc4ccc(C(C)C)nc4)c3C3CC3)cc2)c1C] | 9-21 | 10.53 | 483.6 | 484.2 | [M + H]⁺ | 2 |
| 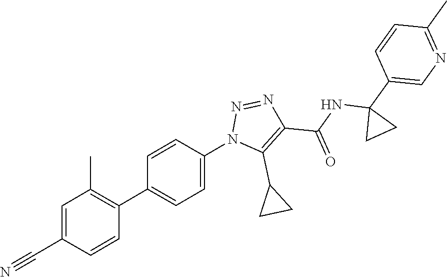 [SMILES: Cc1ccc(C2(NC(=O)c3nnn(-c4ccc(-c5ccc(C#N)cc5C)cc4)c3C3CC3)CC2)cn1] | 9-22 | 11.27 | 474.6 | 474.9 | [M + H]⁺ | 2 |
| 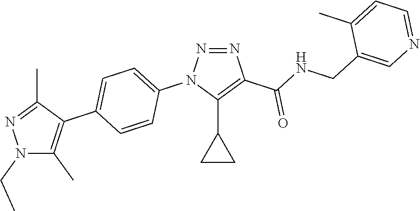 [SMILES: CCn1nc(C)c(-c2ccc(-n3nnc(C(=O)NCc4cnccc4C)c3C3CC3)cc2)c1C] | 9-23 | 10.1 | 455.6 | 456 | [M + H]⁺ | 2 |
| 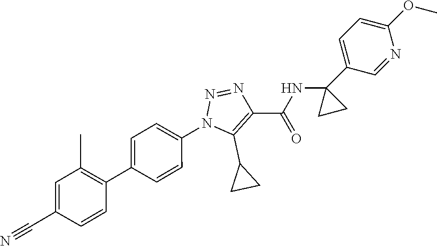 [SMILES: COc1ccc(C2(NC(=O)c3nnn(-c4ccc(-c5ccc(C#N)cc5C)cc4)c3C3CC3)CC2)cn1] | 9-24 | 12.63 | 490.6 | 490.7 | [M + H]⁺ | 2 |
| 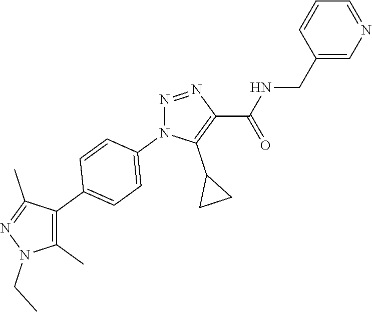 [SMILES: CCn1nc(C)c(-c2ccc(-n3nnc(C(=O)NCc4cccnc4)c3C3CC3)cc2)c1C] | 9-25 | 9.95 | 441.5 | 442 | [M + H]⁺ | 2 |
| 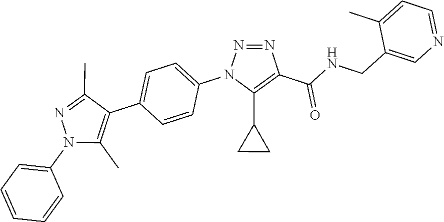 [SMILES: Cc1ccncc1CNC(=O)c1nnn(-c2ccc(-c3c(C)nn(-c4ccccc4)c3C)cc2)c1C1CC1] | 9-26 | 11.44 | 503.6 | 503.7 | [M + H]⁺ | 2 |
| 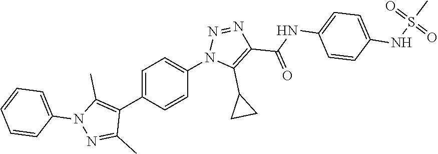 [SMILES: Cc1nn(-c2ccccc2)c(C)c1-c1ccc(-n2nnc(C(=O)Nc3ccc(NS(C)(=O)=O)cc3)c2C2CC2)cc1] | 9-27 | 12.57 | 567.7 | 567.9 | [M + H]⁺ | 2 |
| 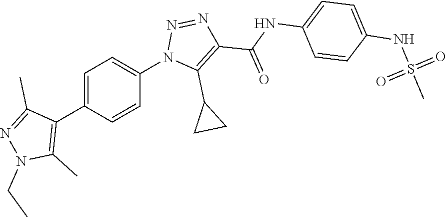 [SMILES: CCn1nc(C)c(-c2ccc(-n3nnc(C(=O)Nc4ccc(NS(C)(=O)=O)cc4)c3C3CC3)cc2)c1C] | 9-28 | 11.67 | 519.6 | 519.9 | [M + H]⁺ | 2 |
| 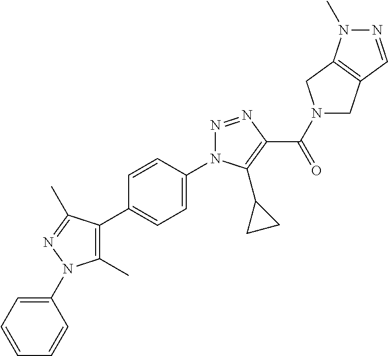 [SMILES: Cc1nn(-c2ccccc2)c(C)c1-c1ccc(-n2nnc(C(=O)N3Cc4cnn(C)c4C3)c2C2CC2)cc1] | 9-29 | 12.6 | 504.6 | 504.7 | [M + H]⁺ | 2 |
| 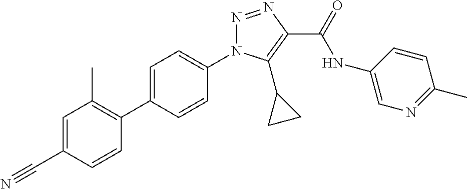 [SMILES: Cc1ccc(NC(=O)c2nnn(-c3ccc(-c4ccc(C#N)cc4C)cc3)c2C2CC2)cn1] | 9-30 | 11.77 | 434.5 | 435 | [M + H]⁺ | 2 |
| 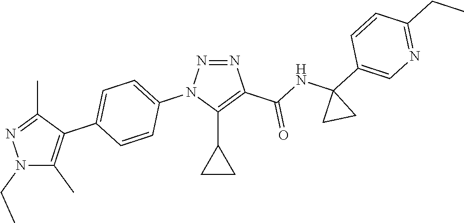 [SMILES: CCc1ccc(C2(NC(=O)c3nnn(-c4ccc(-c5c(C)nn(CC)c5C)cc4)c3C3CC3)CC2)cn1] | 9-31 | 10.64 | 495.6 | 496 | [M + H]⁺ | 2 |
| 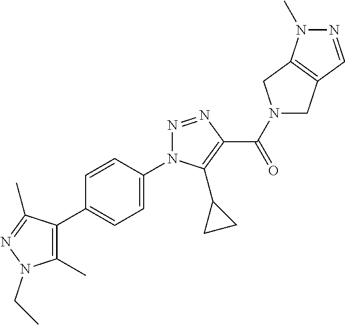 [SMILES: CCn1nc(C)c(-c2ccc(-n3nnc(C(=O)N4Cc5cnn(C)c5C4)c3C3CC3)cc2)c1C] | 9-32 | 11.63 | 456.6 | 457.1 | [M + H]⁺ | 2 |
| 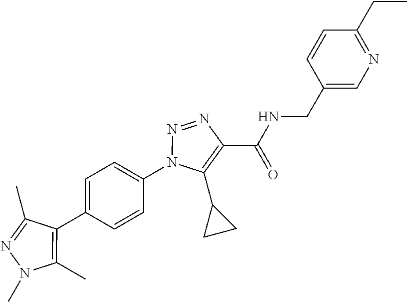 | 9-33 | 10.28 | 481.6 | 482.2 | [M + H]⁺ | 2 |
| 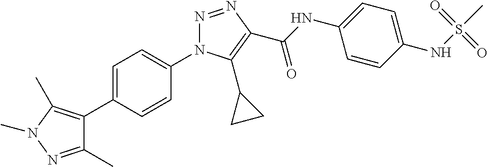 [SMILES: Cc1nn(C)c(C)c1-c1ccc(-n2nnc(C(=O)Nc3ccc(NS(C)(=O)=O)cc3)c2C2CC2)cc1] | 9-34 | 11.34 | 505.6 | 505.8 | [M + H]⁺ | 2 |
| 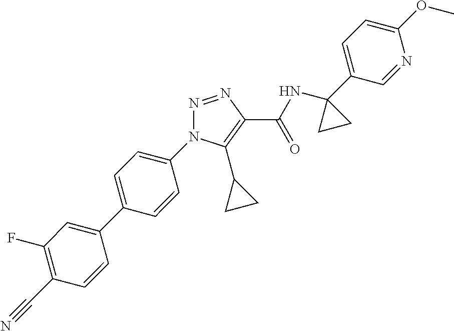 [SMILES: COc1ccc(C2(NC(=O)c3nnn(-c4ccc(-c5ccc(C#N)c(F)c5)cc4)c3C3CC3)CC2)cn1] | 9-35 | 12.37 | 494.5 | 495 | [M + H]⁺ | 2 |
| 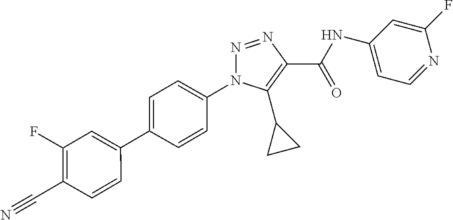 [SMILES: N#Cc1ccc(-c2ccc(-n3nnc(C(=O)Nc4ccnc(F)c4)c3C3CC3)cc2)cc1F] | 9-37 | 12.87 | 442.4 | 443 | [M + H]⁺ | 2 |
| 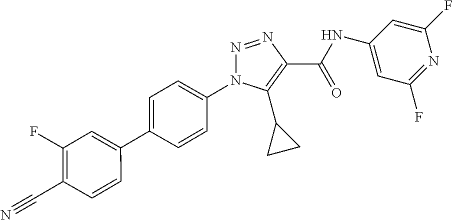 [SMILES: N#Cc1ccc(-c2ccc(-n3nnc(C(=O)Nc4cc(F)nc(F)c4)c3C3CC3)cc2)cc1F] | 9-37 | 13.2 | 460.4 | 460.8 | [M + H]⁺ | 2 |

### Example 10

### Synthesis of Compound 10-1 and Other Representative Compounds

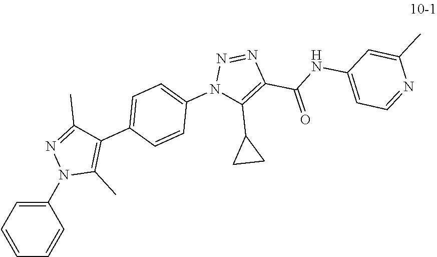 [SMILES: Cc1cc(NC(=O)c2nnn(-c3ccc(-c4c(C)nn(-c5ccccc5)c4C)cc3)c2C2CC2)ccn1]

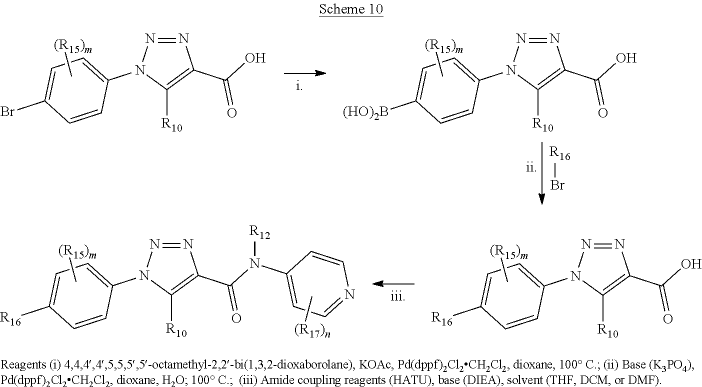

### Step 10-1. Synthesis of 1-(4-boronophenyl)-5-cyclopropyl-1H-1,2,3-triazole-4-carboxylic acid (INT 10A)

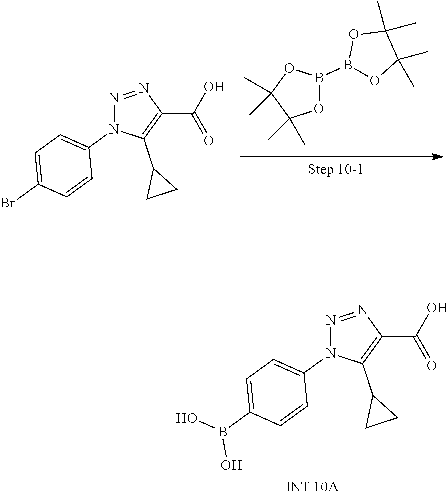

Dioxane (4 mL) was added to a reaction vial containing 1-(4-bromophenyl)-5-cyclopropyl-1H-1,2,3-triazole-4-carboxylic acid (1740 mg, 5.65 mmol), 4,4,4′,4′,5,5,5′,5′-octamethyl-2,2′-bi(1,3,2-dioxaborolane) (2870 mg, 11.29 mmol), Pd(dppf)Cl₂·CH₂Cl₂ (461 mg, 0.56 mmol) and KOAc (1662 mg, 16.95 mmol). The reaction mixture was deoxygenated using N₂ gas for 1 min, stirred and heated at 100° C. for 2 h, and then concentrated. The resulting residue was dissolved in DMSO (5 mL) and purified over RP-chromatography (MeOH/H₂O) to provide 1200 mg (78%) of 1-(4-boronophenyl)-5-cyclopropyl-1H-1,2,3-triazole-4-carboxylic acid (INT 10A) as an off-white solid. LCMS-ESI (m/z) calculated for C₁₂H₁₂BN₃O₄: 273.1; found 274.1 [M+H]⁺, t_{R}=4.7 min (Method 3)

### Step 10-2. Synthesis of 5-cyclopropyl-1-(4-(3,5-dimethyl-1-phenyl-1H-pyrazol-4-yl)phenyl)-1H-1,2,3-triazole-4-carboxylic acid (INT 10B)

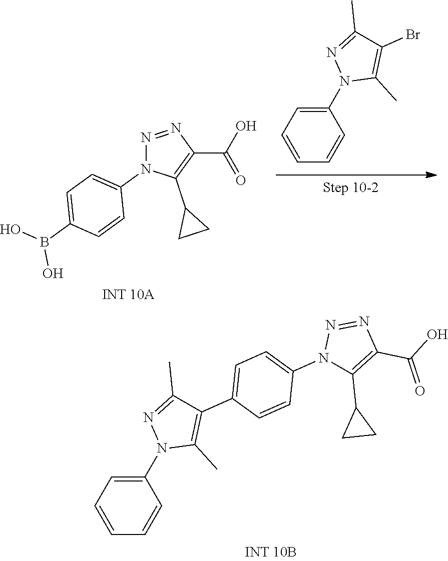

To a vial containing INT 10A (245 mg, 0.90 mmol) were added 4-bromo-3,5-dimethyl-1-phenyl-1H-pyrazole (269.3 mg, 1.08 mmol), Pd(dppf)Cl₂—CH₂Cl₂ (73.3 mg, 0.090 mmol), K₃PO₄ (571 mg, 2.69 mmol), dioxane (3 mL) and H₂O (1 mL). After degassing with N₂ bubbling for 1 min, the reaction mixture was heated to 100° C. and stirred for 2 h. The reaction mixture was directly purified by RP—SiO₂ chromatography (MeOH/H₂O) to afford 358 mg, (72.5%) of 5-cyclopropyl-1-(4-(3,5-dimethyl-1-phenyl-1H-pyrazol-4-yl)phenyl)-1H-1,2,3-triazole-4-carboxylic acid (INT 10B) as a light brown solid. LCMS-ESI (m/z) calculated for C₂₃H₂₁N₅O₂: 399.1; found 400.0 [M+H]⁺, t_{R}=6.0 min (Method 3). ¹H NMR (400 MHz, DMSO-D₆) δ 7.76 (d, J=8.0 Hz, 2H), 7.42-7.61 (m, 7H), 2.31 (s, 3H), 2.19 (s, 3H), 2.11 (m, 1H), 0.94 (m, 2H), 0.72 (m, 2H).

### Step 10-3. Synthesis of 5-cyclopropyl-1-(4-(3,5-dimethyl-1-phenyl-1H-pyrazol-4-yl)phenyl)-N-(2-methylpyridin-4-yl)-1H-1,2,3-triazole-4-carboxamide (Compound 10-1)

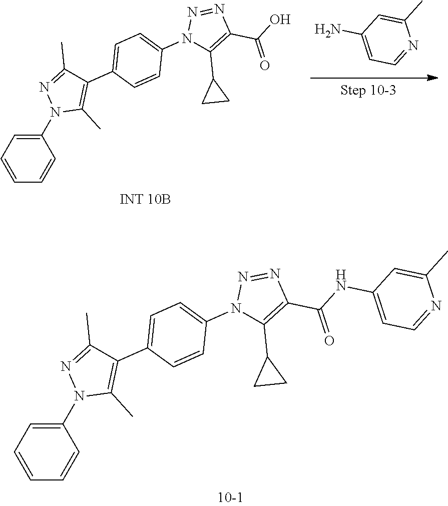

To a stirring solution of INT 10B (40 mg, 0.10 mmol) in DMF (0.8 mL) was added HATU (41.9 mg, 0.11 mmol). After stirring for 15 min, 2-methylpyridin-4-amine (11.9 mg, 0.11 mmol) and DIEA (38 mg, 0.30 mmol) were added. After stirring for 4 h, the reaction mixture diluted with EA and washed with H₂O, NaHCO₃ and brine, then dried (Na₂SO₄), filtered, concentrated, and purified by SiO₂ chromatography (EA/hexanes and MeOH/DCM) to afford 4 mg (8.2%) of 5-cyclopropyl-1-(4-(3,5-dimethyl-1-phenyl-1H-pyrazol-4-yl) phenyl)-N-(2-methylpyridin-4-yl)-1H-1,2,3-triazole-4-carboxamide (Compound 10-1). LCMS-ESI (m/z) calculated for C₂₉H₂₇N₇O: 489.2; found 490.0 [M+H]⁺, t_{R}=11.96 min (Method 2). ¹H NMR (400 MHz, DMSO-D₆) δ 9.93 (s, 1H), 8.51 (s, 1H), 8.05 (s, 1H), 7.93 (s, 1H), 7.50-7.79 (m, 8H), 7.42 (m, 1H), 2.96 (s, 3H), 2.43 (s, 3H), 2.36 (s, 3H), 2.05 (t, J=8.0 Hz, 1H), 1.32 (d, J=8.0 Hz, 2H), 1.20 (t, J=8.0 Hz, 2H).

The compounds listed in Table 10 were made using the procedures of Scheme 10:

TABLE 10

| | | Purity | | | | |
|---|---|---|---|---|---|---|
| | Cpd | RT | | Observed | | Purity |
| Structure | No. | (min) | MW | m/z | Ion | Method |
| 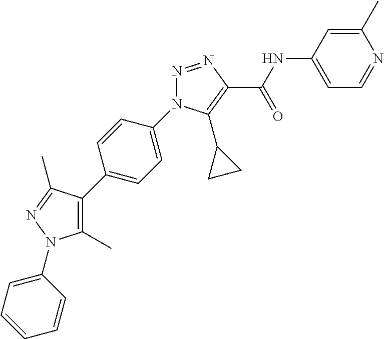 [SMILES: Cc1cc(NC(=O)c2nnn(-c3ccc(-c4c(C)nn(-c5ccccc5)c4C)cc3)c2C2CC2)ccn1] | 10-1 | 12.02 | 489.6 | 490 | [M + H]⁺ | 2 |
| 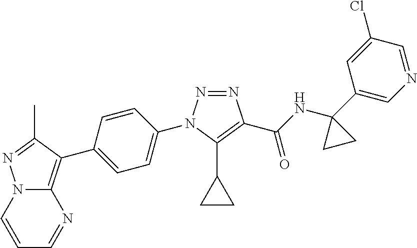 [SMILES: Cc1nn2cccnc2c1-c1ccc(-n2nnc(C(=O)NC3(c4cncc(Cl)c4)CC3)c2C2CC2)cc1] | 10-2 | 12.44 | 511.0 | 511.1 | [M + H]⁺ | 2 |
| 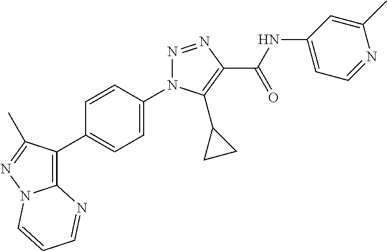 [SMILES: Cc1cc(NC(=O)c2nnn(-c3ccc(-c4c(C)nn5cccnc45)cc3)c2C2CC2)ccn1] | 10-3 | 11.25 | 450.5 | 451.1 | [M + H]⁺ | 2 |
| 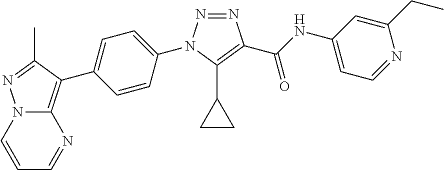 [SMILES: CCc1cc(NC(=O)c2nnn(-c3ccc(-c4c(C)nn5cccnc45)cc3)c2C2CC2)ccn1] | 10-4 | 11.6 | 464.5 | 464.9 | [M + H]⁺ | 2 |
| 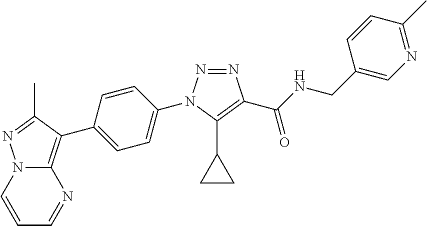 [SMILES: Cc1ccc(CNC(=O)c2nnn(-c3ccc(-c4c(C)nn5cccnc45)cc3)c2C2CC2)cn1] | 10-5 | 10.32 | 464.5 | 465.1 | [M + H]⁺ | 2 |
| 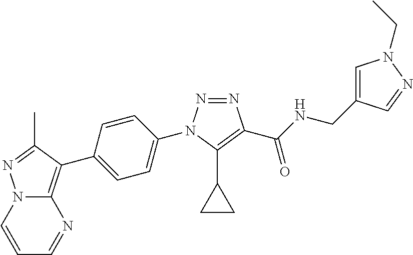 [SMILES: CCn1cc(CNC(=O)c2nnn(-c3ccc(-c4c(C)nn5cccnc45)cc3)c2C2CC2)cn1] | 10-6 | 11.67 | 467.5 | 468.1 | [M + H]⁺ | 2 |
| 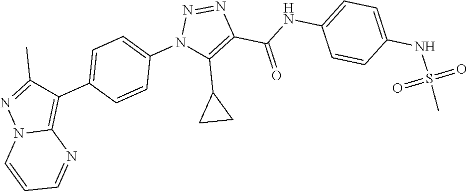 [SMILES: Cc1nn2cccnc2c1-c1ccc(-n2nnc(C(=O)Nc3ccc(NS(C)(=O)=O)cc3)c2C2CC2)cc1] | 10-7 | 11.84 | 528.6 | 529 | [M + H]⁺ | 2 |
| 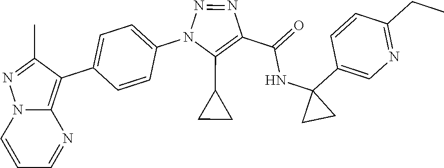 [SMILES: CCc1ccc(C2(NC(=O)c3nnn(-c4ccc(-c5c(C)nn6cccnc56)cc4)c3C3CC3)CC2)cn1] | 10-8 | 10.82 | 504.6 | 504.9 | [M + H]⁺ | 2 |
| 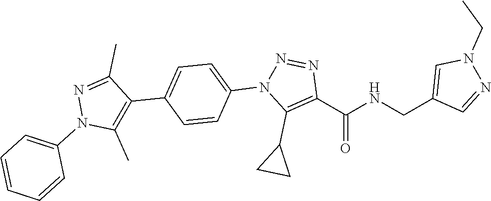 [SMILES: CCn1cc(CNC(=O)c2nnn(-c3ccc(-c4c(C)nn(-c5ccccc5)c4C)cc3)c2C2CC2)cn1] | 10-9 | 12.58 | 506.6 | 506.7 | [M + H]⁺ | 2 |
| 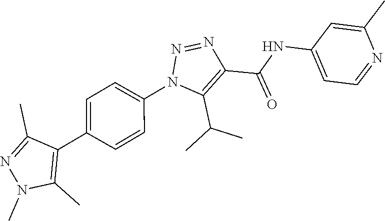 [SMILES: Cc1cc(NC(=O)c2nnn(-c3ccc(-c4c(C)nn(C)c4C)cc3)c2C(C)C)ccn1] | 10-10 | 0.826 | 429.5 | 430.2 | [M + H]⁺ | 4 |
| 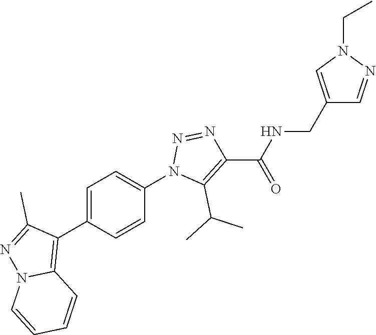 [SMILES: CCn1cc(CNC(=O)c2nnn(-c3ccc(-c4c(C)nn5ccccc45)cc3)c2C(C)C)cn1] | 10-11 | 0.924 | 468.6 | 469.4 | [M + H]⁺ | 4 |
| 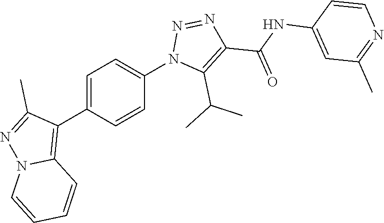 [SMILES: Cc1cc(NC(=O)c2nnn(-c3ccc(-c4c(C)nn5ccccc45)cc3)c2C(C)C)ccn1] | 10-12 | 1.009 | 451.5 | 452.1 | [M + H]⁺ | 5 |

### Example 11

### Synthesis of Compound 11-1 and Other Representative Compounds

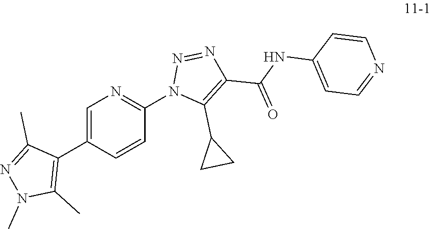 [SMILES: Cc1nn(C)c(C)c1-c1ccc(-n2nnc(C(=O)Nc3ccncc3)c2C2CC2)nc1]

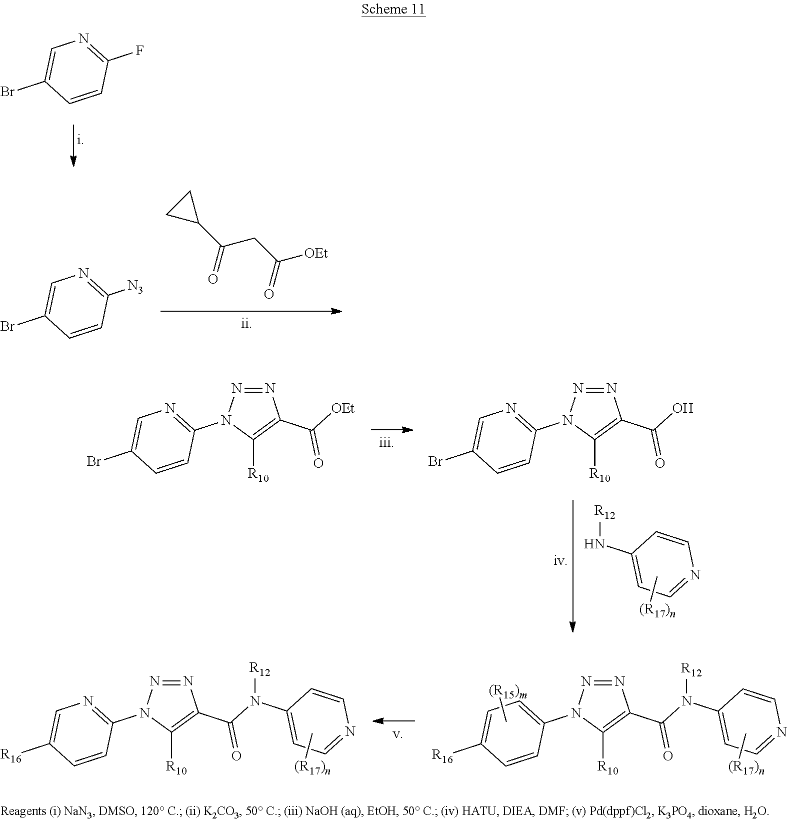

### Step 11-1. Synthesis of 2-azido-5-bromopyridine (INT 11A)

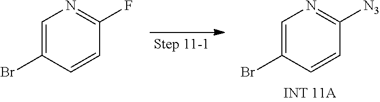 [SMILES: Fc1ccc(Br)cn1 ; [N-]=[N+]=Nc1ccc(Br)cn1]

To a solution of 5-bromo-2-fluoropyridine (1500 mg, 8.5 mmol), in DMSO (12 mL) was added NaN₃ (1.1 g, 17 mmol). After stirring at 120° C. for 16 h, the reaction mixture was diluted with EA, washed with brine, dried (Na₂SO₄), filtered and partially concentrated to provide crude 2-azido-5-bromopyridine (INT 11A) (assumed 100% conversion). LCMS-ESI (m/z) calculated for C₅H₃BrN₄: 197.9; found 199.2 [M+H]⁺, t_{R}˜=4.83 min (Method 3).

### Step 11-2 Synthesis of ethyl 1-(5-bromopyridin-2-yl)-5-cyclopropyl-1H-1,2,3-triazole-4-carboxylate (NIT 11B)

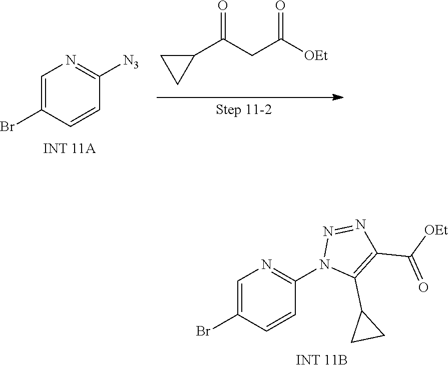

To a stirring solution of INT 11A (1.90 g, 9.54 mmol) in DMSO (6 mL) were added K₂CO₃ (2.64 g, 9.54 mmol) and ethyl 3-cyclopropyl-3-oxopropanoate (1.49 mg, 9.55 mmol). After heating at 50° C. for 1.5 h, the reaction mixture was cooled and was acidified with 3M HCl. The mixture was diluted with NaHCO₃, extracted with EA and the organic layers were washed with brine, dried (Na₂SO₄), filtered, concentrated, and purified by SiO₂ chromatography (EA/hex) to afford 690 mg (21.4%) of ethyl 1-(5-bromopyridin-2-yl)-5-cyclopropyl-1H-1,2,3-triazole-4-carboxylate (INT 11B). LCMS-ESI (m/z) calculated for C₁₃H₁₃BrN₄O: 336; found 336.9 [M+H]⁺, t_{R}=6.21 min (Method 3).

### Step 11-3 Synthesis of 1-(5-bromopyridin-2-yl)-5-cyclopropyl-1H-1,2,3-triazole-4-carboxylic acid (INT 11C)

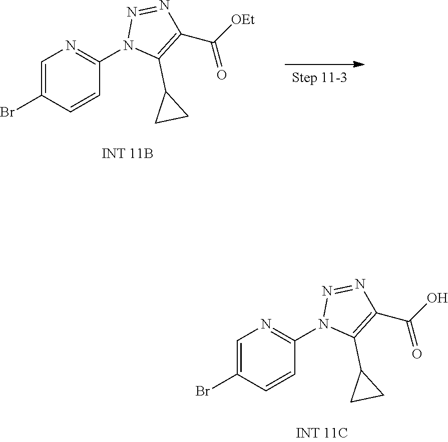 [SMILES: CCOC(=O)c1nnn(-c2ccc(Br)cn2)c1C1CC1 ; O=C(O)c1nnn(-c2ccc(Br)cn2)c1C1CC1]

To a stirring solution of INT 11B (690 mg 2.04 mmol) in EtOH (30 mL) and H₂O (5 mL) was added 1M aqueous NaOH (6.14 mL, 6.14 mmol). After heating at 50° C. for 40 min, the reaction mixture was cooled and concentrated to remove the EtOH. The resulting solution was diluted with H₂O and washed with EA. The resulting aqueous layer was neutralized with 6M HCl, extracted with EA, and the organic layers were washed with brine, dried (Na₂SO₄), filtered, and concentrated to afford 460 mg (73%) of 1-(5-bromopyridin-2-yl)-5-cyclopropyl-1H-1,2,3-triazole-4-carboxylic acid (INT 11C). LCMS-ESI (m/z) calculated for C₁₁H₁₉BrN₄O₂: 307.9; found 309.0 [M+H]⁺, t_{R}=5.74 min (Method 3).

### Step 11-4 Synthesis of 1-(5-bromopyridin-2-yl)-5-cyclopropyl-N-(pyridin-4-yl)-1H-1,2,3-triazole-4-carboxamide (INT 11D)

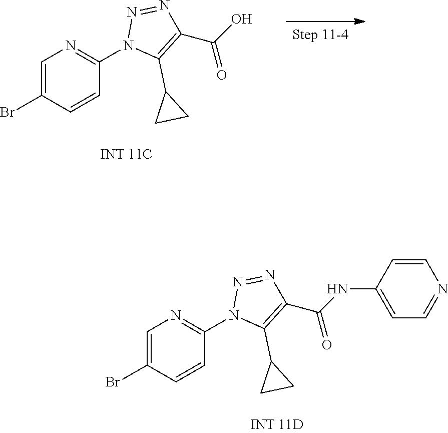 [SMILES: O=C(O)c1nnn(-c2ccc(Br)cn2)c1C1CC1 ; O=C(Nc1ccncc1)c1nnn(-c2ccc(Br)cn2)c1C1CC1]

To a stirring solution of INT 11C (100 mg 0.32 mmol) in DMF (2 mL) was added HATU (129 mg, 0.34 mmol). After stirring for 1 min, DIEA (0.17 mL, 0.97 mmol) and pyridin-4-amine (33 mg, 0.36 mmol) were added to the mixture. After stirring at rt for 3 h, the reaction mixture was diluted with EA, washed with aqueous 1M NaOH and brine, then dried (Na₂SO₄), filtered, concentrated, and purified by SiO₂ chromatography (EA/hexanes) to afford 71 mg (57%) of 1-(5-bromopyridin-2-yl)-5-cyclopropyl-N-(pyridin-4-yl)-1H-1,2,3-triazole-4-carboxamide (INT 11D). LCMS-ESI (m/z) calculated for C₁₆H₁₃BrN₆O: 384.2; found 385.0 [M+H]⁺, t_{R}=5.47 min (Method 3).

### Step 11-5 Synthesis of 5-cyclopropyl-N-(pyridin-4-yl)-1-(5-(1,3,5-trimethyl-1H-pyrazol-4-yl)pyridin-2-yl)-1H-1,2,3-triazole-4-carboxamide (Compound 11-1)

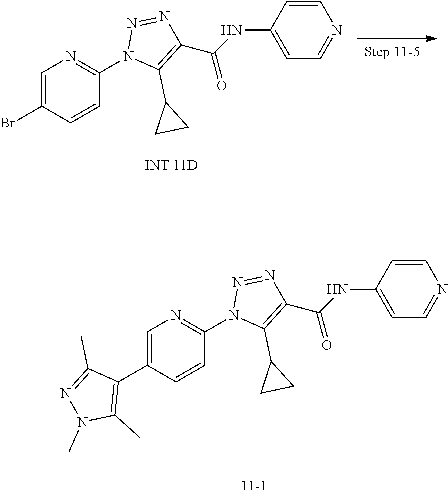 [SMILES: O=C(Nc1ccncc1)c1nnn(-c2ccc(Br)cn2)c1C1CC1 ; Cc1nn(C)c(C)c1-c1ccc(-n2nnc(C(=O)Nc3ccncc3)c2C2CC2)nc1]

To a sealable tube were added INT 11 D (70 mg 0.18 mmol), 1,3,5-trimethyl-4-(4,4,5,5-tetramethyl-1,3,2-dioxaborolan-2-yl)-1H-pyrazole (64 mg, 0.27 mmol), Pd(dppf)Cl₂ (13 mg, 0.02 mmol), and K₃PO₄ (116 mg, 0.55 mmol), followed by dioxane (2 mL) and H₂O (1 mL). After sealing the tube and heating at 110° C. for 1.5 h, the mixture was cooled to rt, diluted with saturated NaHCO₃, and extracted with EA. The organic layer was washed with brine, dried (Na₂SO₄), filtered, concentrated and purified by SiO₂ chromatography (EA/hexane and MeOH/DCM) to afford 38 mg (51%) of 5-cyclopropyl-N-(pyridin-4-yl)-1-(5-(1,3,5-trimethyl-1H-pyrazol-4-yl)pyridin-2-yl)-1H-1,2,3-triazole-4-carboxamide (Compound 11-1). LCMS-ESI (m/z) calculated for C₂₂H₂₂N₈O: 414.2; found 415.2 [M+H]⁺, t_{R}=9.61 min (Method 2). ¹H NMR (400 MHz, DMSO-D₆) δ 10.91 (s, 1H), 8.64 (s, 1H), 8.49 (d, J=8.0 Hz, 2H), 8.11 (d, J=8.0 Hz, 1H), 7.93 (d, J=8.0 Hz, 1H), 7.87 (d, J=8.0 Hz, 2H), 3.76 (s, 3H), 2.40 (m, 1H), 2.37 (s, 3H), 2.30 (s, 3H), 0.97 (m, 4H).

The compounds listed in the Table 11 was made using the procedures of Scheme 11.

TABLE 11

| | | Purity | | | | Purity |
|---|---|---|---|---|---|---|
| Structure | Cpd No. | RT (min) | MW | Observed m/z | Ion | Method |
| 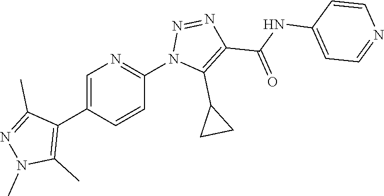 [SMILES: Cc1nn(C)c(C)c1-c1ccc(-n2nnc(C(=O)Nc3ccncc3)c2C2CC2)nc1] | 11-1 | 9.99 | 414.5 | 415.2 | [M + H]⁺ | 2 |
| 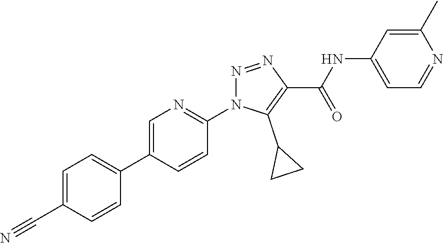 [SMILES: Cc1cc(NC(=O)c2nnn(-c3ccc(-c4ccc(C#N)cc4)cn3)c2C2CC2)ccn1] | 11-2 | 8.59 | 421.5 | 421.7 | [M + H]⁺ | 2 |

### Example 12

### Synthesis of Compound 12-1 and Other Representative Compounds

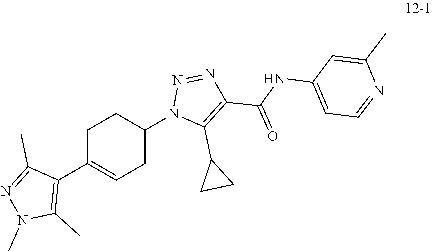 [SMILES: Cc1cc(NC(=O)c2nnn(C3CC=C(c4c(C)nn(C)c4C)CC3)c2C2CC2)ccn1]

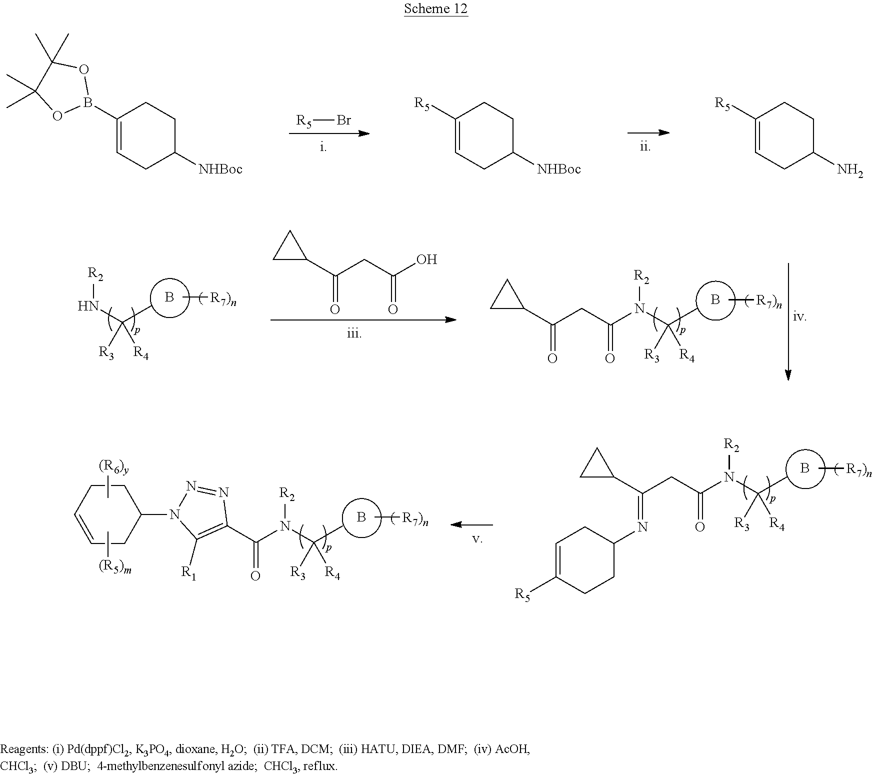

### Step 12-1. Synthesis of tert-butyl (4-(1,3,5-trimethyl-1H-pyrazol-4-yl)cyclohex-3-en-1-yl)carbamate (INT 12A)

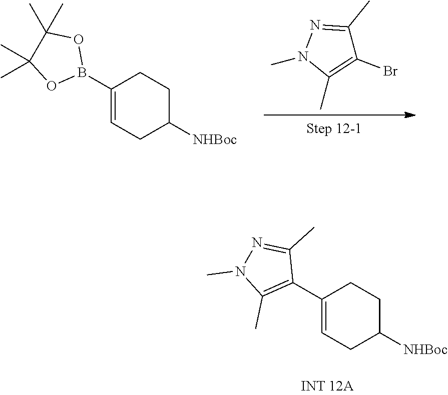

To a sealable tube were added Pd(dppf)₂Cl₂ (45 mg, 0.06 mmol), tert-butyl (4-(4,4,5,5-tetramethyl-1,3,2-dioxaborolan-2-yl)cyclohex-3-en-1-yl)carbamate (400 mg, 1.24 mmol), 4-bromo-1,3,5-trimethyl-1H-pyrazole (258 mg, 0.1.36 mmol), K₃PO₄ (788 mg, 3.8 mmol), dioxane (10 mL) and H₂O (2 mL). After stirring for 1 h at 110° C. the mixture was diluted with EA, washed with saturated aqueous NaHCO₃ and brine, dried (Na₂SO₄s), filtered, and concentrated to provide a crude product that was purified by SiO₂ chromatography (EA/hex, then MeOH/DCM) to afford 259 mg (73%) of tert-butyl (4-(1,3,5-trimethyl-1H-pyrazol-4-yl)cyclohex-3-en-1-yl)carbamate (INT 12A) LCMS-ESI (m/z) calculated for C₁₇H₂₇N₃O₂: 305.2; found 306.2 [M+H]⁺, t_{R}˜=5.86 min (Method 3).

### Step 12-2. Synthesis of 4-(1,3,5-trimethyl-1H-pyrazol-4-yl)cyclohex-3-en-1-amine (INT 12B)

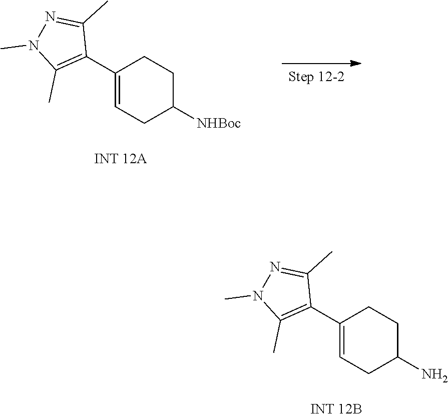 [SMILES: Cc1nn(C)c(C)c1C1=CCC(NC(=O)OC(C)(C)C)CC1 ; Cc1nn(C)c(C)c1C1=CCC(N)CC1]

A solution of INT 12A (175 mg, 0.57 mmol) in DCM (2 mL) and TFA (2 mL) was stirred at room temperature for 5 hr. The reaction mixture was concentrated, dissolved in EA, washed with brine, dried (Na₂SO₄), filtered and concentrated to provide 33 mg (28%) of 4-(1,3,5-trimethyl-1H-pyrazol-4-yl)cyclohex-3-en-1-amine (INT 12B). LCMS-ESI (m/z) calculated for C₁₂H₁₉N₃: 205.2, found 206.2 [M+H]⁺, t_{R}=3.45 min (Method 3).

### Step 12-3. Synthesis of 3-cyclopropyl-N-(2-methylpyridin-4-yl)-3-oxopropanamide (INT 12B)

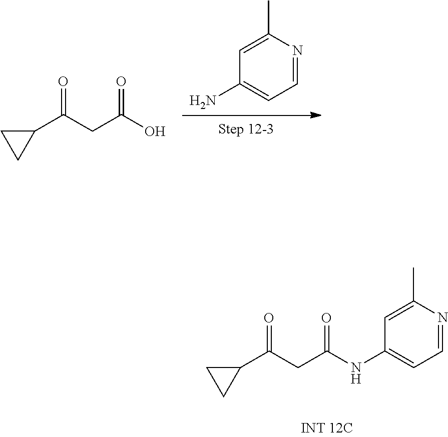

To a round bottom flask containing a stirring solution of INT 2A (3.0 g, 13.8 mmol) in DMF (10 mL) and DIEA (5.3 g, 41.5 mmol) was added 2-methylpyridin-4-amine (1.4 g, 15.2 mmol), and HATU (5.78 g, 15.2 mmol). The reaction mixture was stirred for 12 h, diluted with EA, washed with H₂O, sat aq. NaHCO₃, H₂O, and brine, then dried (Na₂SO₄), filtered, concentrated, and purified by SiO₂ chromatography (EA/hex) to provide 2.5 g (83.0%) of 3-cyclopropyl-3-oxo-N-(pyridin-4-yl)propanamide (INT 12C). ¹H NMR (400 MHz, CDCl₃) S 10.47 (s, 1H), 8.35 (d, J=4.0 Hz, 1H), 7.70 (s, 1H), 7.58 (d, J=4.0 Hz, 1H), 3.85 (s, 2H), 2.67 (s, 3H), 2.03-2.09 (m, 1H), 1.17-1.25 (m, 2H), 1.09-1.14 (m, 2H).

### Step 12-4. Synthesis of 3-cyclopropyl-N-(2-methylpyridin-4-yl)-3-((4-(1,3,5-trimethyl-1H-pyrazol-4-yl)cyclohex-3-en-1-yl)imino)propanamide (INT 12D)

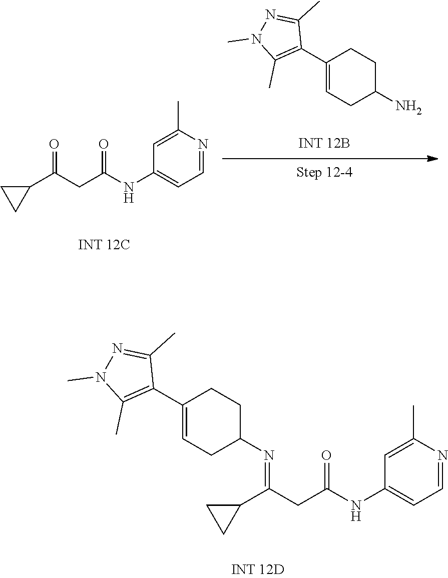 [SMILES: Cc1cc(NC(=O)CC(=O)C2CC2)ccn1 ; Cc1nn(C)c(C)c1C1=CCC(N)CC1 ; Cc1cc(NC(=O)C/C(=N/C2CC=C(c3c(C)nn(C)c3C)CC2)C2CC2)ccn1]

To a stirring solution of INT 12B (372.5 mg, 1.8 mmol) and INT 12C (360 mg, 1.65 mmol) in CHCl₃ (25 mL) was added AcOH (109 mg, 1.81 mmol). After stirring at reflux (61° C.) for 16 h, the reaction mixture was diluted with EA, washed with sat aq. NaHCO₃ and brine, then dried (Na₂SO₄), filtered, concentrated, and purified by SiO₂ chromatography (EA/hex) to provide 510 mg (76.0%) of 3-cyclopropyl-N-(2-methylpyridin-4-yl)-3-((4-(1,3,5-trimethyl-1H-pyrazol-4-yl)cyclohex-3-en-1-yl)imino)propanamide (INT 12D). LCMS-ESI (m/z) calculated for C₂₄H₃₁N₅O: 405.2; found 406.0 [M+H]⁺, t_{R}=5.7 min (Method 3).

### Step 12-5. Synthesis of 5-cyclopropyl-N-(2-methylpyridin-4-yl)-1-(4-(1,3,5-trimethyl-1H-pyrazol-4-yl)cyclohex-3-en-1-yl)-1H-1,2,3-triazole-4-carboxamide (Compound 12-1.)

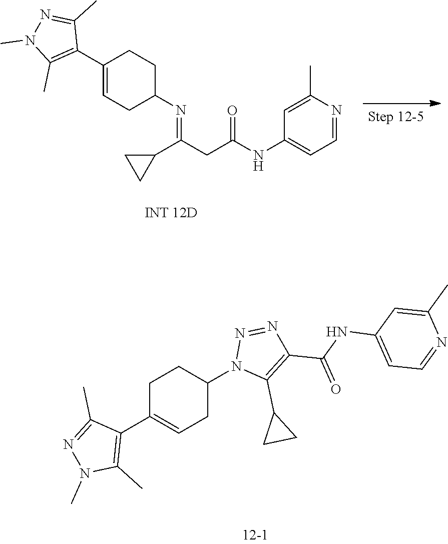 [SMILES: Cc1cc(NC(=O)C/C(=N/C2CC=C(c3c(C)nn(C)c3C)CC2)C2CC2)ccn1 ; Cc1cc(NC(=O)c2nnn(C3CC=C(c4c(C)nn(C)c4C)CC3)c2C2CC2)ccn1]

To a stirring solution of INT 12D (510 mg, 1.3 mmol) in CH₃CN (25 mL) at 0° C. were added DBU (191 mg, 1.3 mmol) and 4-methylbenzenesulfonyl azide (248 mg, 1.3 mmol). After stirring at rt for 12 h, the reaction mixture was heated to reflux for an additional 12 h. The reaction mixture was then concentrated, diluted with EA, washed with H₂O and brine, then dried (Na₂SO₄), filtered, concentrated and purified by SiO₂ chromatography (EA/hex) and reverse phase-chromatography (MeOH/H₂O) to provide 160 mg (29.5%4) of 5-cyclopropyl-N-(2-methylpyridin-4-yl)-1-(4-(1,3,5-trimethyl-1H-pyrazol-4-yl)cyclohex-3-en-1-yl)-1H-1,2,3-triazole-4-carboxamide (Compound 12-1). LCMS-ESI (m/z) calculated for C₂₄H₂₉N₇O: 431.2, found 432.1 [M+H]⁺, t_{R}=10.03 min (Method 2). ¹H NMR (400 MHz, CDCl₃) δ 9.27 (s, 1H), 8.42 (d, J=8.0 Hz, 1H), 7.58 (s, 1H), 7.40 (d, J=8.0 Hz, 1H), 5.58 (d, J=4.0 Hz, 1H), 4.91 (in, 1H), 3.74 (s, 3H), 3.0 (m, 1H), 2.70 (m, 2H), 2.65 (s, 3H), 2.55 (m, 2H), 2.47 (m, 1H), 2.21 (s, 6H), 1.85 (m, 1H), 1.29 (d, J=8.0 Hz, 2H), 1.20 (t, J=8.0 Hz, 2H).

The compounds listed in Table 12 were made using the procedures of Scheme 12.

TABLE 12

| | | Purity | | | | |
|---|---|---|---|---|---|---|
| | Cpd | RT | | Observed | | Purity |
| Structure | No. | (min) | MW | m/z | Ion | Method |
| 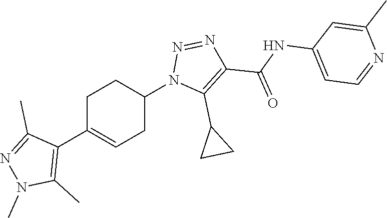 [SMILES: Cc1cc(NC(=O)c2nnn(C3CC=C(c4c(C)nn(C)c4C)CC3)c2C2CC2)ccn1] | 12-1 | 10.03 | 431.5 | 432.1 | [M + H]⁺ | 2 |
| 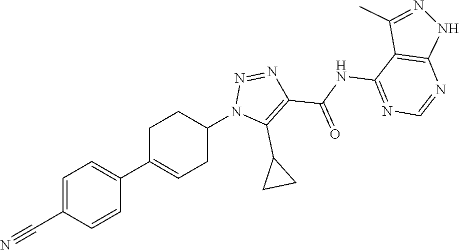 [SMILES: Cc1n[nH]c2ncnc(NC(=O)c3nnn(C4CC=C(c5ccc(C#N)cc5)CC4)c3C3CC3)c12] | 12-2 | 11.68 | 465.5 | 465.5 | [M + H]⁺ | 2 |
| 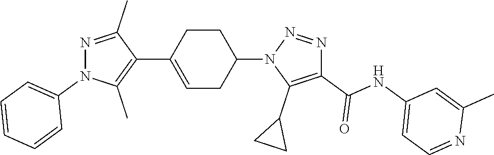 [SMILES: Cc1cc(NC(=O)c2nnn(C3CC=C(c4c(C)nn(-c5ccccc5)c4C)CC3)c2C2CC2)ccn1] | 12-3 | 12.02 | 493.6 | 494.1 | [M + H]⁺ | 2 |
| 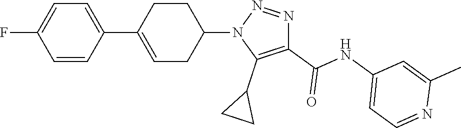 [SMILES: Cc1cc(NC(=O)c2nnn(C3CC=C(c4ccc(F)cc4)CC3)c2C2CC2)ccn1] | 12-4 | 12.15 | 417.5 | 417.7 | [M + H]⁺ | 2 |
| 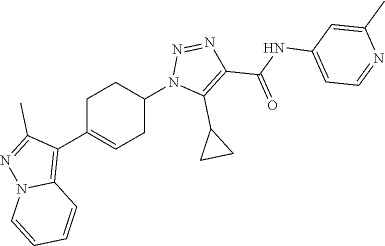 [SMILES: Cc1cc(NC(=O)c2nnn(C3CC=C(c4c(C)nn5ccccc45)CC3)c2C2CC2)ccn1] | 12-5 | 11.59 | 453.6 | 454.3 | [M + H]⁺ | 2 |
| 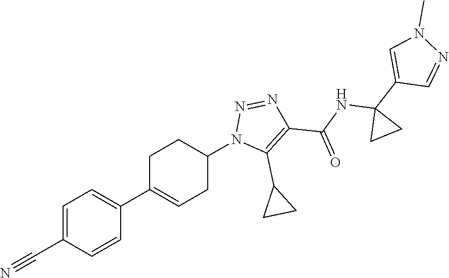 [SMILES: Cn1cc(C2(NC(=O)c3nnn(C4CC=C(c5ccc(C#N)cc5)CC4)c3C3CC3)CC2)cn1] | 12-6 | 11.99 | 453.6 | 454.1 | [M + H]⁺ | 2 |
| 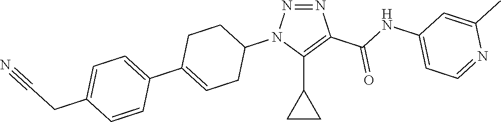 [SMILES: Cc1cc(NC(=O)c2nnn(C3CC=C(c4ccc(CC#N)cc4)CC3)c2C2CC2)ccn1] | 12-7 | 11.17 | 438.5 | 439.1 | [M + H]⁺ | 2 |
| 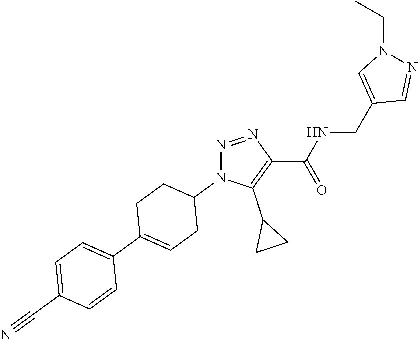 [SMILES: CCn1cc(CNC(=O)c2nnn(C3CC=C(c4ccc(C#N)cc4)CC3)c2C2CC2)cn1] | 12-8 | 11.87 | 441.5 | 442 | [M + H]⁺ | 2 |
| 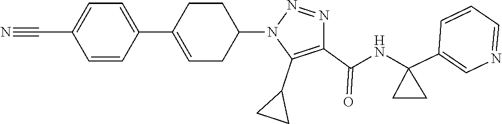 [SMILES: N#Cc1ccc(C2=CCC(n3nnc(C(=O)NC4(c5cccnc5)CC4)c3C3CC3)CC2)cc1] | 12-9 | 10.88 | 450.5 | 450.8 | [M + H]⁺ | 2 |

### Example 13

### Synthesis of Compound 13-1 and Other Representative Compounds

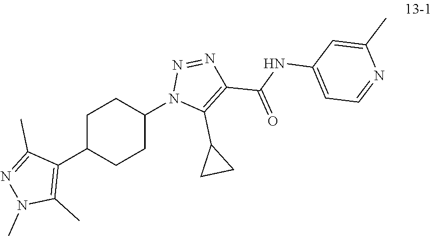 [SMILES: Cc1cc(NC(=O)c2nnn(C3CCC(c4c(C)nn(C)c4C)CC3)c2C2CC2)ccn1]

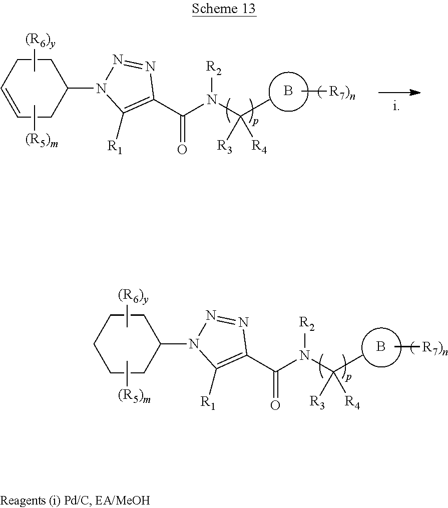

### Step 13-1. Synthesis of 5-cyclopropyl-N-(2-methylpyridin-4-yl)-1-(4-(1,3,5-trimethyl-1H-pyrazol-4-yl)cyclohexyl)-1H-1,2,3-triazole-4-carboxamide (Compound 13-1)

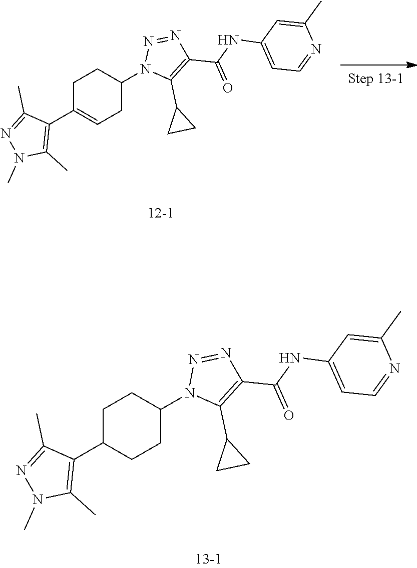 [SMILES: Cc1cc(NC(=O)c2nnn(C3CC=C(c4c(C)nn(C)c4C)CC3)c2C2CC2)ccn1 ; Cc1cc(NC(=O)c2nnn(C3CCC(c4c(C)nn(C)c4C)CC3)c2C2CC2)ccn1]

To a stirring solution of Compound 12-1 (130 mg, 0.3 mmol) in 1:1 EA/MeOH was added 5% Pd/C (39 mg, 0.3 mmol). The reaction mixture was purged with H₂ gas and stirred for 12 h under an atmosphere of H₂ (balloon). The reaction mixture was filtered through celite and concentrated to provide 120 mg (92%) of 5-cyclopropyl-N-(2-methylpyridin-4-yl)-1-(4-(1,3,5-trimethyl-1H-pyrazol-4-yl)cyclohexyl)-1H-1,2,3-triazole-4-carboxamide (Compound 13-1). LCMS-ESI (m/z) calculated for C₂₄H₃₁N₇O: 433.2; found 434.2[M+H]⁺, t_{R}=9.53 min (Method 2). ¹H NMR (400 MHz, CDCl₃): 9.27 (s, 1H), 8.42 (d, J=8.0 Hz, 1H), 7.60 (s, 1H), 7.40 (d, J=8.0 Hz, 1H), 5.10 (s, 1H), 3.71 (s, 3H), 2.63 (m, 1H), 2.58 (s, 3H), 2.44 (m, 2H), 2.37 (s, 6H), 2.26 (m, 2H), 2.11 (m, 2H), 1.85 (m, 1H), 1.63 (m, 2H), 1.29 (d, J=8.0 Hz, 2H), 1.20 (t, J=8.0 Hz, 2H).

The compound listed in Table 13 was made using the procedures of Scheme 13.

TABLE 13

| | | Purity | | | | |
|---|---|---|---|---|---|---|
| | Cpd | RT | | Observed | | Purity |
| Structure | No. | (min) | MW | m/z | Ion | Method |
| 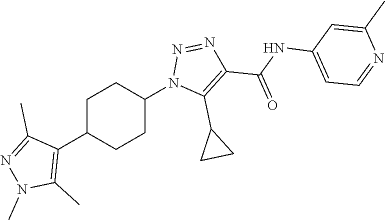 [SMILES: Cc1cc(NC(=O)c2nnn(C3CCC(c4c(C)nn(C)c4C)CC3)c2C2CC2)ccn1] | 13-1 | 9.53 | 433.6 | 434.2 | [M + H]⁺ | 2 |

### Example 14

### Synthesis of Compound 14-15 and Compound 14-16 and Other Representative Compounds

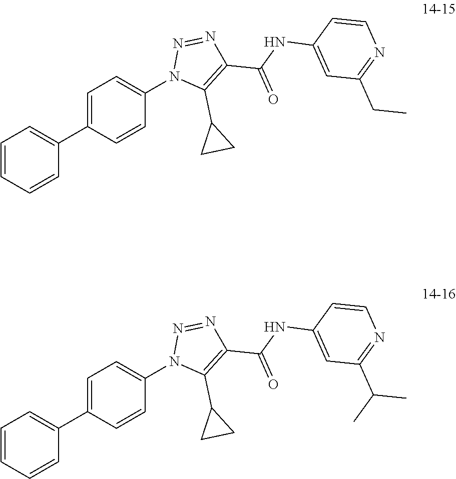 [SMILES: CCc1cc(NC(=O)c2nnn(-c3ccc(-c4ccccc4)cc3)c2C2CC2)ccn1 ; CC(C)c1cc(NC(=O)c2nnn(-c3ccc(-c4ccccc4)cc3)c2C2CC2)ccn1]

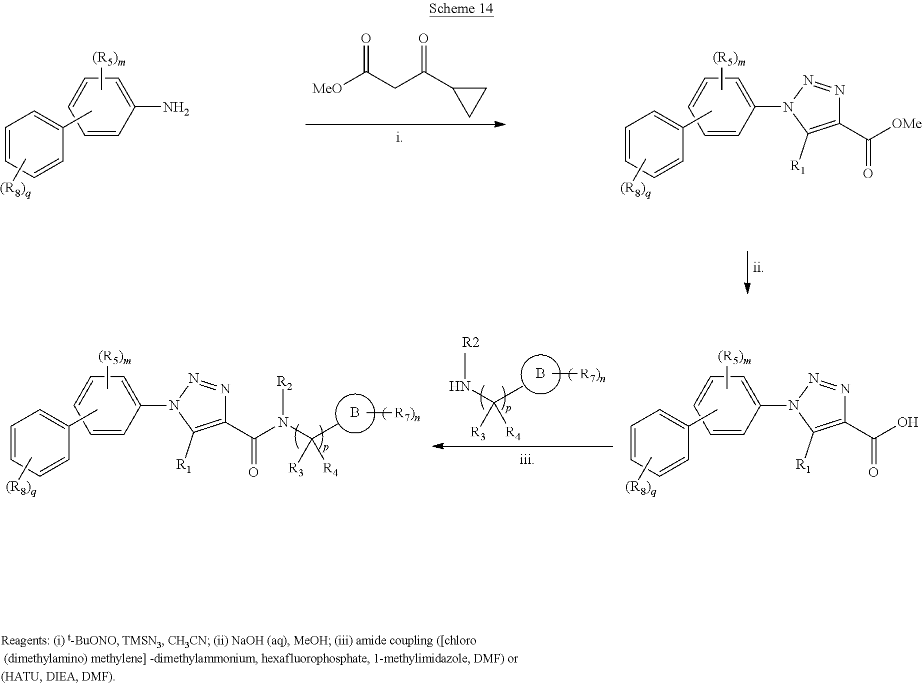

### Step 14-1. Synthesis of methyl 1-([1,1′-biphenyl]-4-yl)-5-cyclopropyl-1H-1,2,3-triazole-4-carboxylate (INT 14A)

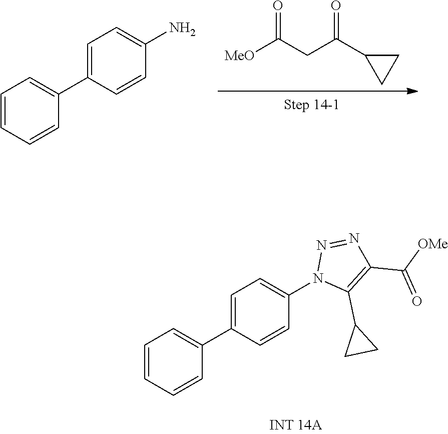 [SMILES: Nc1ccc(-c2ccccc2)cc1 ; COC(=O)CC(=O)C1CC1 ; COC(=O)c1nnn(-c2ccc(-c3ccccc3)cc2)c1C1CC1]

A solution of 4-phenylaniline (10 g, 59.09 mmol) in CH₃CN (160 mL) was cooled to 0° C. in an ice bath. To this stirring mixture were added t-BuONO (8.43 mL, 70.91 mmol) and TMSN₃ (9.33 mL, 70.91 mmol) dropwise. The resulting solution was stirred at 25° C. for 1 h. The reaction mixture was poured into NaHCO₃ (aq.) at 0° C., and then diluted with EA and washed with H₂O. The combined organic layers were washed with brine, dried (Na₂SO₄), filtered and concentrated under reduced pressure to give a residue that was purified by reverse-phase HPLC to afford 10 g (50.2%) of methyl 1-([1,1′-biphenyl]-4-yl)-5-cyclopropyl-1H-1,2,3-triazole-4-carboxylate (INT 14A) as a yellow solid. LCMS-ESI (m/z) calculated for C₁₉H₁₇N₃O₂: 319.4; found 320.0 [M+H]⁺, t_{R}=0.919 min (Method 4). ¹H NMR (400 MHz, CDCl₃) δ=7.71-7.65 (m, 2H), 7.59-7.50 (m, 4H), 7.45-7.38 (m, 2H), 7.36-7.30 (m, 1H), 3.90 (s, 3H), 1.96-1.89 (m, 1H), 1.00-0.93 (m, 2H), 0.86-0.80 (m, 2H).

### Step 14-2. Synthesis of 1-([1,1′-biphenyl]-4-yl)-5-cyclopropyl-1H-1,2,3-triazole-4-carboxylic acid (INT 14B)

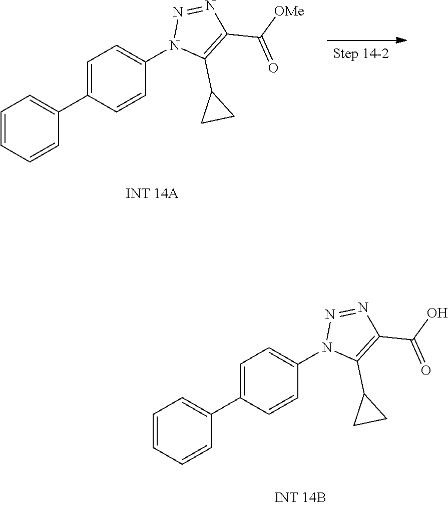 [SMILES: COC(=O)c1nnn(-c2ccc(-c3ccccc3)cc2)c1C1CC1 ; O=C(O)c1nnn(-c2ccc(-c3ccccc3)cc2)c1C1CC1]

To a solution of INT 14A (10 g, 31.31 mmol) in MeOH (50 mL) was added NaOH (1 M, 62.63 mL, 62.63 mmol). After stirring at 15° C. for 1 h, the reaction mixture was diluted with EA and extracted with H₂O. The organic layers was discarded and the aqueous phase was poured into 1 M HCl, then diluted with EA and washed with H₂O and brine, then dried (Na₂SO₄), filtered and concentrated under reduced pressure to provide 9 g (94%) of crude 1-([1,1′-biphenyl]-4-yl)-5-cyclopropyl-1H-1,2,3-triazole-4-carboxylic acid (INT 14B) as a yellow solid and was used in the next step without further purification. LCMS-ESI (m/z) calculated for C₁₈H₁₅N₃O₂: 305.3; found 306.1 [M+H]⁺, t_{R}=0.854 min (Method 4). ¹H NMR (400 MHz, DMSO-D₆) 5=13.09 (br s, 1H), 7.92 (d, J=8.5 Hz, 2H), 7.77 (t, J=7.8 Hz, 4H), 7.57-7.48 (m, 2H), 7.47-7.38 (m, 1H), 2.13 (tt, J=5.4, 8.6 Hz, 1H), 0.97-0.83 (m, 2H), 0.77-0.63 (m, 2H)).

### Step 14-3a. Synthesis of 1-([1,1′-biphenyl]-4-vi)-5-cyclopropyl-N-(2-ethylpyridin-4-yl-1H-1,2,3-triazole-4-carboxamide (Compound 14-15)

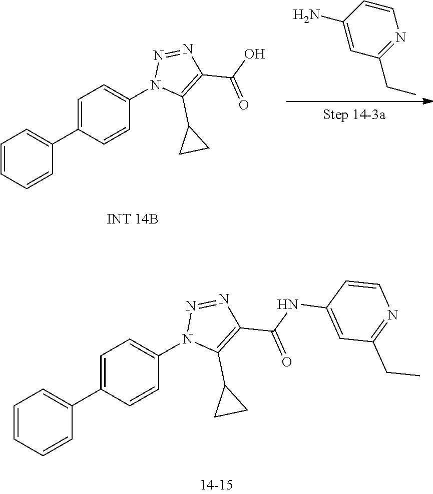

To a mixture of INT 14B (50 mg, 0.164 mmol) and 2-ethylpyridin-4-amine (24.01 mg, 0.197 mmol) in DMF (2 mL) was added [chloro (dimethylamino) methylene]-dimethylammonium hexafluorophosphate (55.14 mg, 0.197 mmol) and 1-methylimidazole (41.68 mg, 0.51 mmol). After stirring at 60° C. for 15 h, the mixture was further stirred at 80° C. for 5 h. The mixture was diluted with aqueous NaHCO₃ and extracted with EA (2×). The combined organic layers were dried (Na₂SO₄), filtered and concentrated under reduced pressure to a residue that was purified by reverse-phase preparatory scale HPLC to provide 3.4 mg (5.1%) of 1-([1,1′-biphenyl]-4-yl)-5-cyclopropyl-N-(2-ethylpyridin-4-yl)-1H-1,2,3-triazole-4-carboxamide (Compound 14-15) as an off-white solid. LCMS-ESI (m/z) calculated for C₂₅H₂₃N₅O: 409.5; found 410.2 [M+H]+, t_{R}=0.875 min (Method 4). ¹H NMR (400 MHz, CDCl₃) δ=9.51 (br s, 1H), 8.49 (d, J=5.9 Hz, 1H), 7.82 (d, J=8.5 Hz, 2H), 7.73 (br s, 1H), 7.69-7.57 (m, 5H), 7.56-7.48 (m, 2H), 7.48-7.41 (m, 1H), 3.14-2.85 (m, 2H), 2.06 (tt, J=5.6, 8.5 Hz, 1H), 1.41 (t, J=7.6 Hz, 3H), 1.24-1.17 (m, 2H), 1.16-1.08 (m, 2H).

### Step 14-3b. Synthesis of 1-([1,1′-biphenyl]-4-yl)-5-cyclopropyl-N-(2-isopropylpyridin-4-yl)-1H-1,2,3-triazole-4-carboxamide (Compound 14-16)

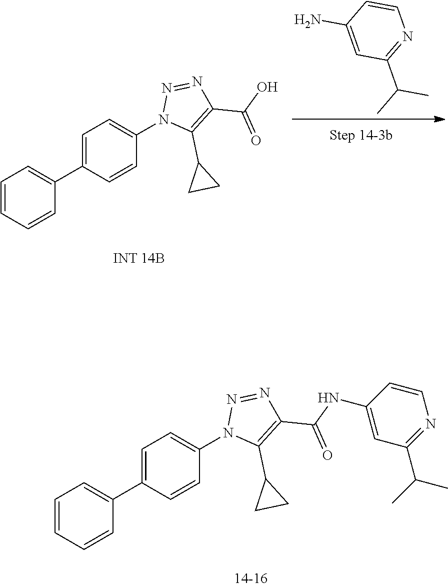

To a mixture of INT 14B (30 mg, 0.098 mmol) and 2-isopropylpyridin-4-amine (16.06 mg, 0.12 mmol) in DMF (1 mL) were added HATU (56.04 mg, 0.147 mmol) and DIEA (51.34 μL, 0.295 mmol). After stirring at 20° C. for 1 h, the mixture was diluted with aqueous NaHCO₃ and extracted with EA (2×). The combined organic layers were washed with saturated aqueous NaCl, dried (Na₂SO₄), filtered and concentrated under reduced pressure to a residue that was purified by reverse-phase prep HPLC to provide 5.4 mg (12.8%) of 1-([1,1′-biphenyl]-4-yl)-5-cyclopropyl-N-(2-isopropylpyridin-4-yl)-1H-1,2,3-triazole-4-carboxamide (Compound 14-16) as a yellow solid. LCMS-ESI (m/z) calculated for C₂₆H₂₅N₅O: 423.5: found 424.3 [M+H]+, t_{R}=0.788 min (Method 4). ¹H NMR (400 MHz, CDCl₃) δ=9.85-9.22 (m, 1H), 8.52 (d, J=5.9 Hz, 1H), 7.82 (d, J=8.3 Hz, 2H), 7.76-7.59 (m, 6H), 7.52 (t, J=7.6 Hz, 2H), 7.48-7.42 (m, 1H), 3.49-3.17 (m, 1H), 2.11-2.02 (m, 1H), 1.42 (br d, J=6.7 Hz, 6H), 1.23-1.17 (m, 2H), 1.15-1.10 (m, 2H).

The compounds list in Table 14 were made using the procedures of Scheme 14 (final step either 14-3a or 14-3b):

TABLE 14

| | | Purity | | | | |
|---|---|---|---|---|---|---|
| | Cpd | RT | | Observed | | Purity |
| Structure | No. | (min) | MW | m/z | Ion | Method |
| 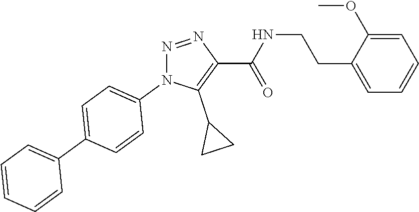 [SMILES: COc1ccccc1CCNC(=O)c1nnn(-c2ccc(-c3ccccc3)cc2)c1C1CC1] | 14-1 | 1.048 | 438.5 | 439.3 | [M + H]⁺ | 4 |
| 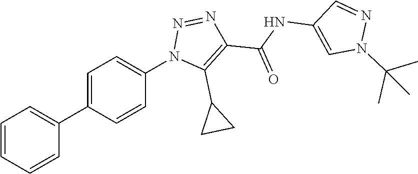 [SMILES: CC(C)(C)n1cc(NC(=O)c2nnn(-c3ccc(-c4ccccc4)cc3)c2C2CC2)cn1] | 14-2 | 1.034 | 426.5 | 427.2 | [M + H]⁺ | 4 |
| 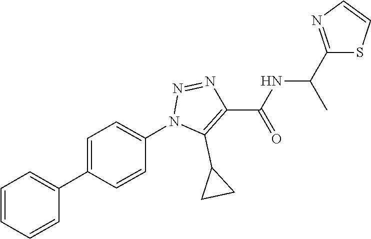 [SMILES: CC(NC(=O)c1nnn(-c2ccc(-c3ccccc3)cc2)c1C1CC1)c1nccs1] | 14-3 | 0.911 | 415.5 | 416.1 | [M + H]⁺ | 4 |
| 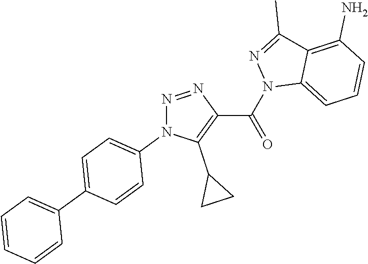 [SMILES: Cc1nn(C(=O)c2nnn(-c3ccc(-c4ccccc4)cc3)c2C2CC2)c2cccc(N)c12] | 14-4 | 0.970 | 434.5 | 435.3 | [M + H]⁺ | 4 |
| 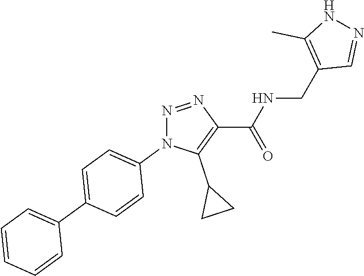 [SMILES: Cc1[nH]ncc1CNC(=O)c1nnn(-c2ccc(-c3ccccc3)cc2)c1C1CC1] | 14-5 | 0.866 | 398.5 | 399.3 | [M + H]⁺ | 4 |
| 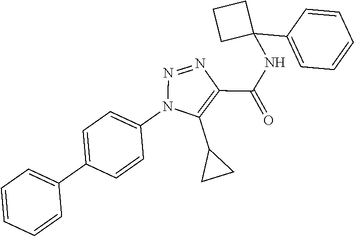 [SMILES: O=C(NC1(c2ccccc2)CCC1)c1nnn(-c2ccc(-c3ccccc3)cc2)c1C1CC1] | 14-6 | 1.2 | 434.5 | 435.3 | [M + H]⁺ | 5 |
| 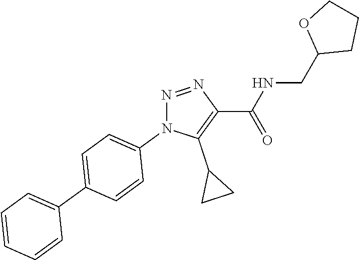 [SMILES: O=C(NCC1CCCO1)c1nnn(-c2ccc(-c3ccccc3)cc2)c1C1CC1] | 14-7 | 1.045 | 388.5 | 389.3 | [M + H]⁺ | 5 |
| 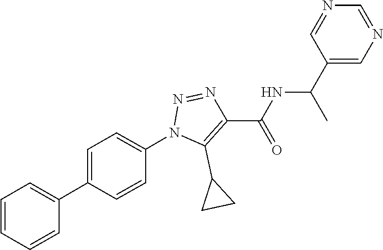 [SMILES: CC(NC(=O)c1nnn(-c2ccc(-c3ccccc3)cc2)c1C1CC1)c1cncnc1] | 14-8 | 0.903 | 410.5 | 411.1 | [M + H]⁺ | 4 |
| 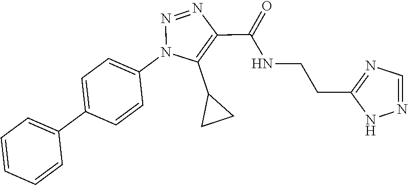 [SMILES: O=C(NCCc1ncn[nH]1)c1nnn(-c2ccc(-c3ccccc3)cc2)c1C1CC1] | 14-9 | 0.838 | 399.5 | 400.1 | [M + H]⁺ | 4 |
| 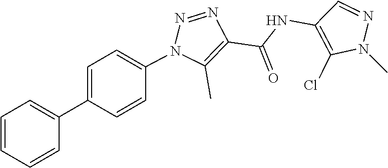 | 14-10 | 0.968 | 418.9 | 418.9 | [M + H]⁺ | 4 |
| 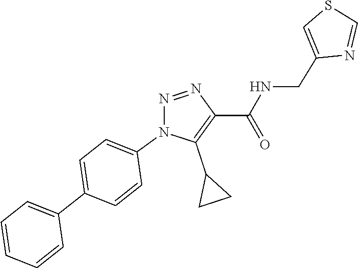 [SMILES: O=C(NCc1cscn1)c1nnn(-c2ccc(-c3ccccc3)cc2)c1C1CC1] | 14-11 | 0.991 | 401.5 | 402.1 | [M + H]⁺ | 5 |
| 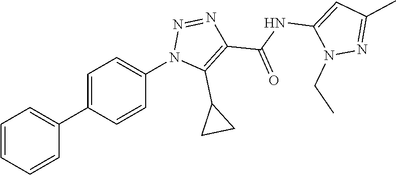 [SMILES: CCn1nc(C)cc1NC(=O)c1nnn(-c2ccc(-c3ccccc3)cc2)c1C1CC1] | 14-12 | 0.983 | 412.5 | 413.2 | [M + H]⁺ | 4 |
| 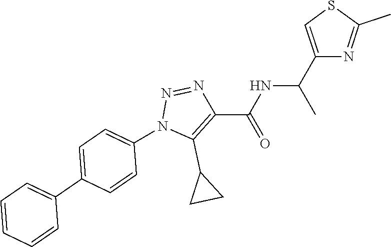 [SMILES: Cc1nc(C(C)NC(=O)c2nnn(-c3ccc(-c4ccccc4)cc3)c2C2CC2)cs1] | 14-13 | 0.948 | 429.5 | 430 | [M + H]⁺ | 4 |
| 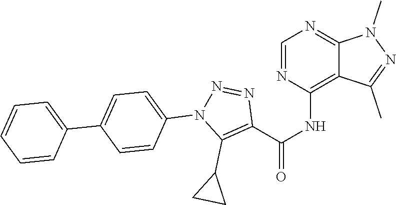 [SMILES: Cc1nn(C)c2ncnc(NC(=O)c3nnn(-c4ccc(-c5ccccc5)cc4)c3C3CC3)c12] | 14-14 | 0.975 | 450.5 | 451.1 | [M + H]⁺ | 5 |
| 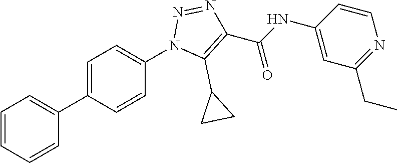 [SMILES: CCc1cc(NC(=O)c2nnn(-c3ccc(-c4ccccc4)cc3)c2C2CC2)ccn1] | 14-15 | 0.875 | 409.5 | 410.2 | [M + H]⁺ | 4 |
| 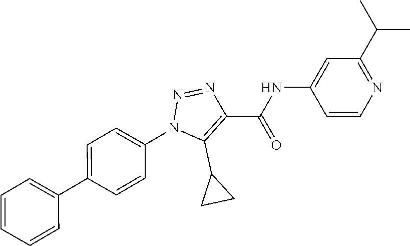 [SMILES: CC(C)c1cc(NC(=O)c2nnn(-c3ccc(-c4ccccc4)cc3)c2C2CC2)ccn1] | 14-16 | 0.788 | 423.5 | 424.3 | [M + H]⁺ | 4 |
| 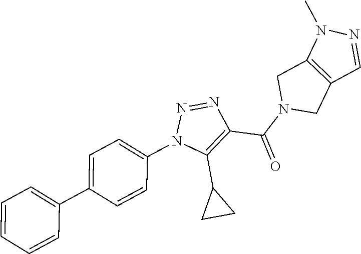 [SMILES: Cn1ncc2c1CN(C(=O)c1nnn(-c3ccc(-c4ccccc4)cc3)c1C1CC1)C2] | 14-17 | 0.903 | 410.5 | 411 | [M + H]⁺ | 4 |
| 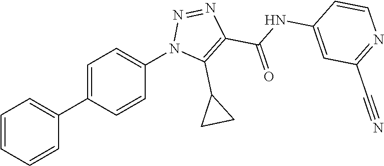 [SMILES: N#Cc1cc(NC(=O)c2nnn(-c3ccc(-c4ccccc4)cc3)c2C2CC2)ccn1] | 14-18 | 0.911 | 405.5 | 406.2 | [M + H]⁺ | 4 |
| 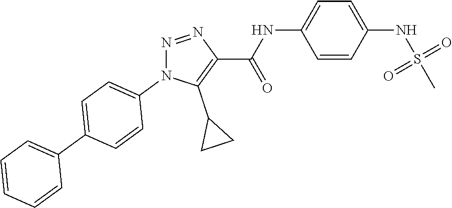 [SMILES: CS(=O)(=O)Nc1ccc(NC(=O)c2nnn(-c3ccc(-c4ccccc4)cc3)c2C2CC2)cc1] | 14-19 | 0.950 | 473.6 | 474.2 | [M + H]⁺ | 5 |
| 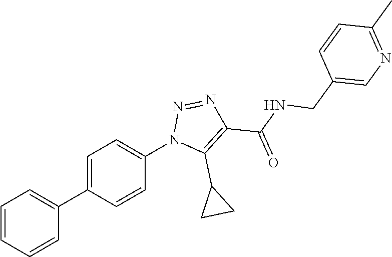 [SMILES: Cc1ccc(CNC(=O)c2nnn(-c3ccc(-c4ccccc4)cc3)c2C2CC2)cn1] | 14-20 | 0.47 | 409.5 | 410 | [M + H]⁺ | 4 |
| 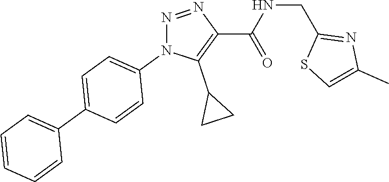 [SMILES: Cc1csc(CNC(=O)c2nnn(-c3ccc(-c4ccccc4)cc3)c2C2CC2)n1] | 14-21 | 1.007 | 415.5 | 416 | [M + H]⁺ | 5 |
| 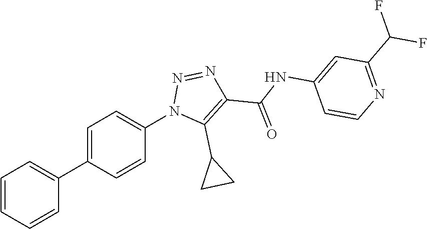 [SMILES: O=C(Nc1ccnc(C(F)F)c1)c1nnn(-c2ccc(-c3ccccc3)cc2)c1C1CC1] | 14-22 | 0.994 | 431.4 | 432.2 | [M + H]⁺ | 4 |
| 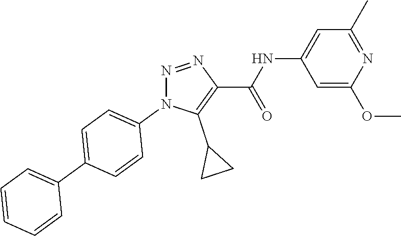 [SMILES: COc1cc(NC(=O)c2nnn(-c3ccc(-c4ccccc4)cc3)c2C2CC2)cc(C)n1] | 14-23 | 0.893 | 425.5 | 426.3 | [M + H]⁺ | 4 |
| 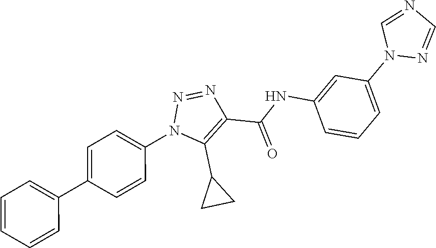 [SMILES: O=C(Nc1cccc(-n2cncn2)c1)c1nnn(-c2ccc(-c3ccccc3)cc2)c1C1CC1] | 14-24 | 0.94 | 447.5 | 448.1 | [M + H]⁺ | 4 |
| 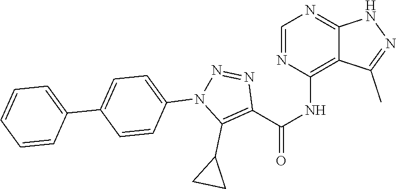 [SMILES: Cc1n[nH]c2ncnc(NC(=O)c3nnn(-c4ccc(-c5ccccc5)cc4)c3C3CC3)c12] | 14-25 | 0.911 | 436.5 | 437.1 | [M + H]⁺ | 5 |
| 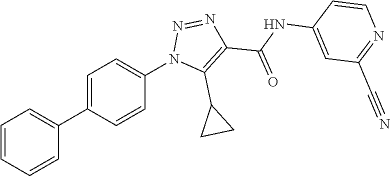 [SMILES: N#Cc1cc(NC(=O)c2nnn(-c3ccc(-c4ccccc4)cc3)c2C2CC2)ccn1] | 14-26 | 1.019 | 406.4 | 407.3 | [M + H]⁺ | 4 |
| 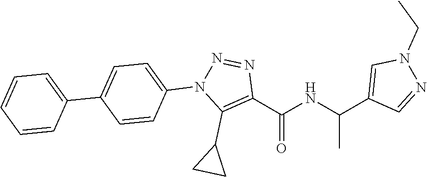 [SMILES: CCn1cc(C(C)NC(=O)c2nnn(-c3ccc(-c4ccccc4)cc3)c2C2CC2)cn1] | 14-27 | 0.948 | 426.5 | 427.3 | [M + H]⁺ | 4 |
| 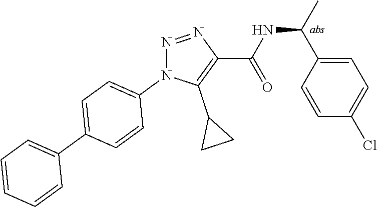 [SMILES: C[C@H](NC(=O)c1nnn(-c2ccc(-c3ccccc3)cc2)c1C1CC1)c1ccc(Cl)cc1] | 14-28 | 1.169 | 442.9 | 443.3 | [M + H]⁺ | 5 |
| 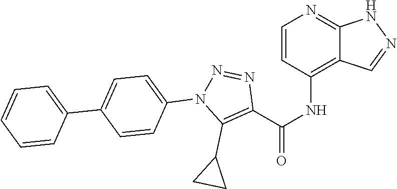 [SMILES: O=C(Nc1ccnc2[nH]ncc12)c1nnn(-c2ccc(-c3ccccc3)cc2)c1C1CC1] | 14-29 | 0.890 | 421.5 | 422.3 | [M + H]⁺ | 4 |
| 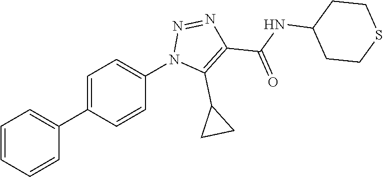 [SMILES: O=C(NC1CCSCC1)c1nnn(-c2ccc(-c3ccccc3)cc2)c1C1CC1] | 14-30 | 1.066 | 404.5 | 405 | [M + H]⁺ | 5 |
| 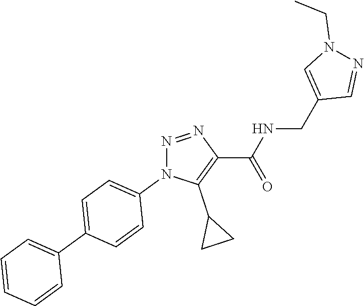 [SMILES: CCn1cc(CNC(=O)c2nnn(-c3ccc(-c4ccccc4)cc3)c2C2CC2)cn1] | 14-31 | 0.927 | 412.5 | 413.1 | [M + H]⁺ | 4 |
| 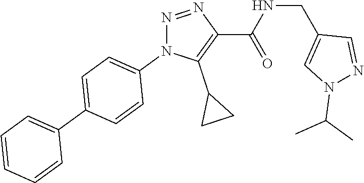 [SMILES: CC(C)n1cc(CNC(=O)c2nnn(-c3ccc(-c4ccccc4)cc3)c2C2CC2)cn1] | 14-32 | 0.918 | 426.5 | 427.2 | [M + H]⁺ | 4 |
| 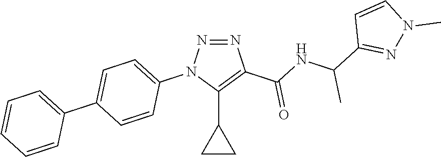 [SMILES: CC(NC(=O)c1nnn(-c2ccc(-c3ccccc3)cc2)c1C1CC1)c1ccn(C)n1] | 14-33 | 0.947 | 412.5 | 413.2 | [M + H]⁺ | 4 |
| 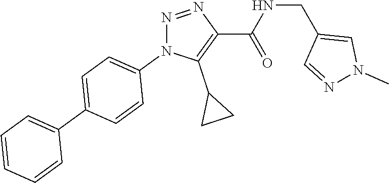 [SMILES: Cn1cc(CNC(=O)c2nnn(-c3ccc(-c4ccccc4)cc3)c2C2CC2)cn1] | 14-34 | 0.903 | 398.5 | 399 | [M + H]⁺ | 4 |
| 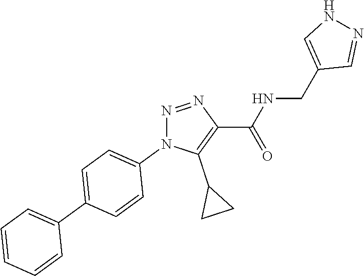 [SMILES: O=C(NCc1cn[nH]c1)c1nnn(-c2ccc(-c3ccccc3)cc2)c1C1CC1] | 14-36 | 0.87 | 384.4 | 385 | [M + H]⁺ | 4 |
| 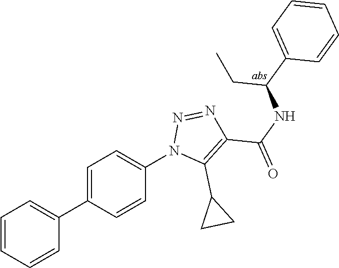 [SMILES: CC[C@H](NC(=O)c1nnn(-c2ccc(-c3ccccc3)cc2)c1C1CC1)c1ccccc1] | 14-37 | 1.154 | 422.5 | 423.2 | [M + H]⁺ | 5 |
| 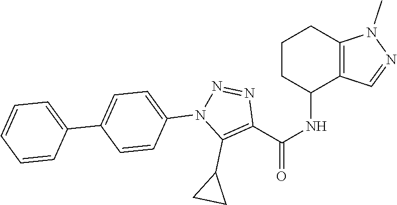 [SMILES: Cn1ncc2c1CCCC2NC(=O)c1nnn(-c2ccc(-c3ccccc3)cc2)c1C1CC1] | 14-38 | 0.934 | 438.5 | 439.3 | [M + H]⁺ | 4 |
| 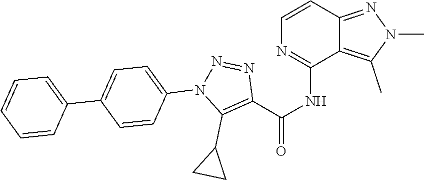 [SMILES: Cc1c2c(NC(=O)c3nnn(-c4ccc(-c5ccccc5)cc4)c3C3CC3)nccc2nn1C] | 14-39 | 0.835 | 449.5 | 450 | [M + H]⁺ | 4 |
| 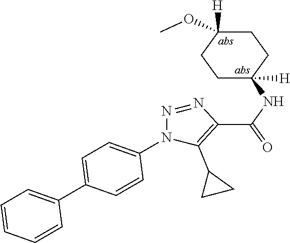 | 14-40 | 1.089 | 416.5 | 417.3 | [M + H]⁺ | 5 |
| 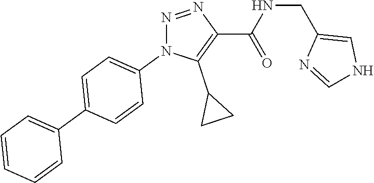 [SMILES: O=C(NCc1c[nH]cn1)c1nnn(-c2ccc(-c3ccccc3)cc2)c1C1CC1] | 14-41 | 0.8 | 384.4 | 385 | [M + H]⁺ | 4 |
| 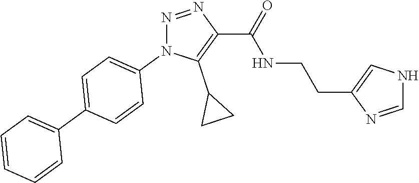 [SMILES: O=C(NCCc1c[nH]cn1)c1nnn(-c2ccc(-c3ccccc3)cc2)c1C1CC1] | 14-42 | 0.795 | 398.5 | 399 | [M + H]⁺ | 4 |
| 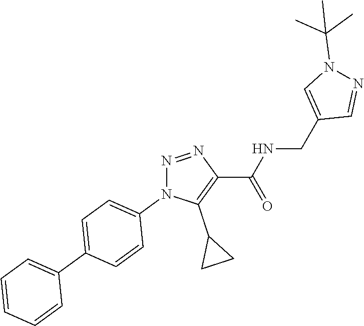 | 14-43 | 0.984 | 454.6 | 455.1 | [M + H]⁺ | 4 |
| 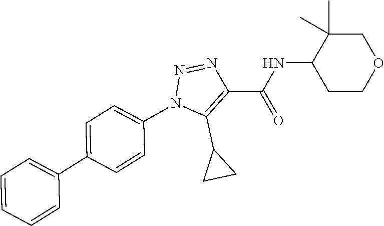 [SMILES: CC1(C)COCCC1NC(=O)c1nnn(-c2ccc(-c3ccccc3)cc2)c1C1CC1] | 14-44 | 1.103 | 416.5 | 417.3 | [M + H]⁺ | 5 |
| 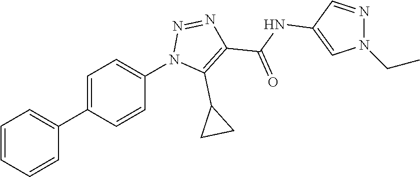 [SMILES: CCn1cc(NC(=O)c2nnn(-c3ccc(-c4ccccc4)cc3)c2C2CC2)cn1] | 14-45 | 1.035 | 398.5 | 399.3 | [M + H]⁺ | 5 |
| 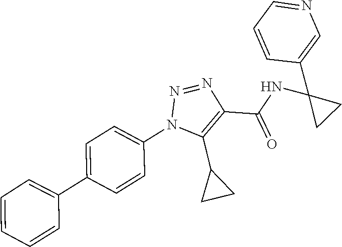 [SMILES: O=C(NC1(c2cccnc2)CC1)c1nnn(-c2ccc(-c3ccccc3)cc2)c1C1CC1] | 14-46 | 1.04 | 421.5 | 422.3 | [M + H]⁺ | 5 |
| 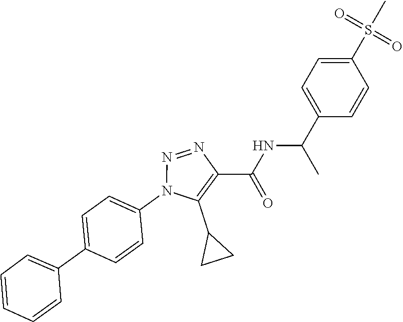 [SMILES: CC(NC(=O)c1nnn(-c2ccc(-c3ccccc3)cc2)c1C1CC1)c1ccc(S(C)(=O)=O)cc1] | 14-47 | 1.081 | 486.6 | 487.3 | [M + H]⁺ | 5 |
| 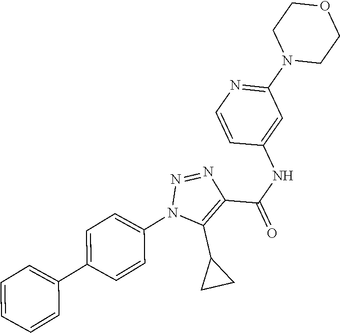 [SMILES: O=C(Nc1ccnc(N2CCOCC2)c1)c1nnn(-c2ccc(-c3ccccc3)cc2)c1C1CC1] | 14-48 | 1.105 | 466.5 | 467.3 | [M + H]⁺ | 5 |
| 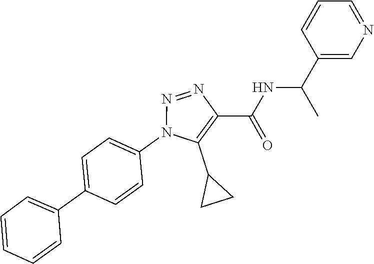 [SMILES: CC(NC(=O)c1nnn(-c2ccc(-c3ccccc3)cc2)c1C1CC1)c1cccnc1] | 14-49 | 0.853 | 409.5 | 410.2 | [M + H]⁺ | 4 |
| 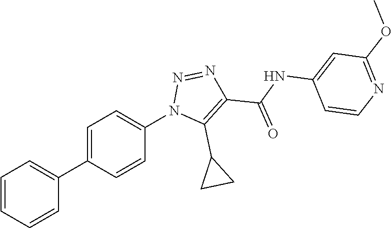 [SMILES: COc1cc(NC(=O)c2nnn(-c3ccc(-c4ccccc4)cc3)c2C2CC2)ccn1] | 14-50 | 0.833 | 411.5 | 412.2 | [M + H]⁺ | 4 |
| 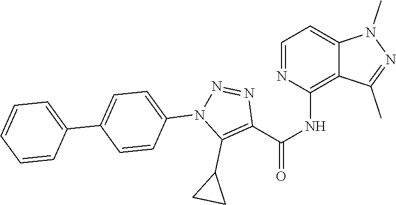 [SMILES: Cc1nn(C)c2ccnc(NC(=O)c3nnn(-c4ccc(-c5ccccc5)cc4)c3C3CC3)c12] | 14-51 | 0.856 | 449.5 | 450 | [M + H]⁺ | 4 |
| 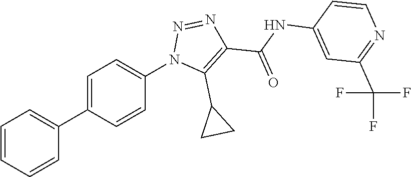 [SMILES: O=C(Nc1ccnc(C(F)(F)F)c1)c1nnn(-c2ccc(-c3ccccc3)cc2)c1C1CC1] | 14-52 | 1.062 | 449.4 | 450.3 | [M + H]⁺ | 4 |
| 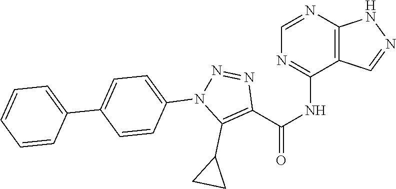 [SMILES: O=C(Nc1ncnc2[nH]ncc12)c1nnn(-c2ccc(-c3ccccc3)cc2)c1C1CC1] | 14-53 | 0.933 | 422.5 | 423 | [M + H]⁺ | 4 |
| 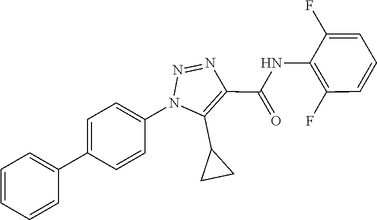 [SMILES: O=C(Nc1c(F)cccc1F)c1nnn(-c2ccc(-c3ccccc3)cc2)c1C1CC1] | 14-54 | 1.021 | 416.4 | 417 | [M + H]⁺ | 4 |
| 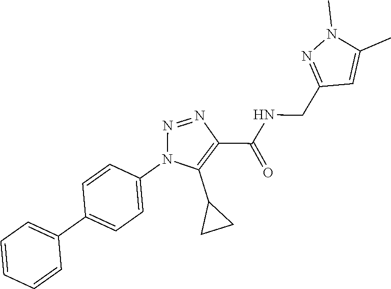 [SMILES: Cc1cc(CNC(=O)c2nnn(-c3ccc(-c4ccccc4)cc3)c2C2CC2)nn1C] | 14-55 | 0.977 | 412.5 | 413 | [M + H]⁺ | 5 |
| 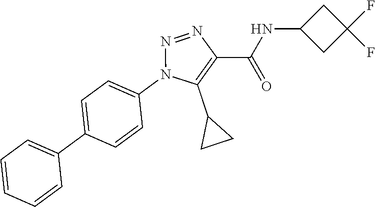 [SMILES: O=C(NC1CC(F)(F)C1)c1nnn(-c2ccc(-c3ccccc3)cc2)c1C1CC1] | 14-56 | 1.1 | 394.4 | 395.2 | [M + H]⁺ | 5 |
| 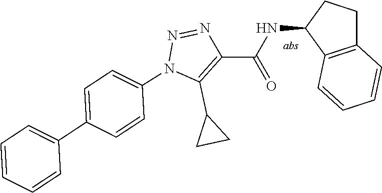 [SMILES: O=C(N[C@H]1CCc2ccccc21)c1nnn(-c2ccc(-c3ccccc3)cc2)c1C1CC1] | 14-57 | 1.133 | 420.5 | 421.2 | [M + H]⁺ | 5 |
| 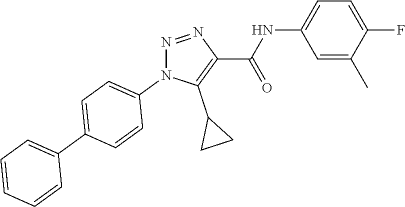 [SMILES: Cc1cc(NC(=O)c2nnn(-c3ccc(-c4ccccc4)cc3)c2C2CC2)ccc1F] | 14-58 | 1.163 | 412.5 | 413.1 | [M + H]⁺ | 5 |
| 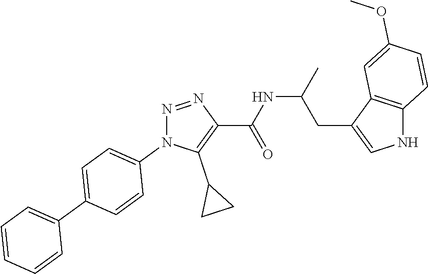 [SMILES: COc1ccc2[nH]cc(CC(C)NC(=O)c3nnn(-c4ccc(-c5ccccc5)cc4)c3C3CC3)c2c1] | 14-59 | 1.085 | 491.6 | 492.3 | [M + H]⁺ | 5 |
| 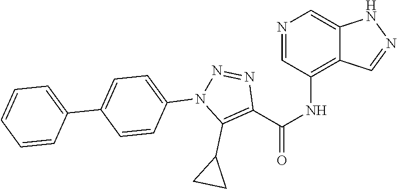 [SMILES: O=C(Nc1cncc2[nH]ncc12)c1nnn(-c2ccc(-c3ccccc3)cc2)c1C1CC1] | 14-60 | 0.840 | 421.5 | 422.3 | [M + H]⁺ | 4 |
| 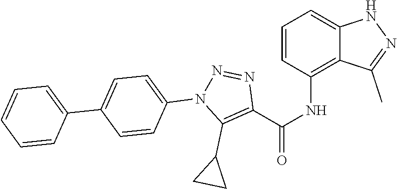 [SMILES: Cc1n[nH]c2cccc(NC(=O)c3nnn(-c4ccc(-c5ccccc5)cc4)c3C3CC3)c12] | 14-61 | 0.958 | 434.5 | 435.1 | [M + H]⁺ | 4 |
| 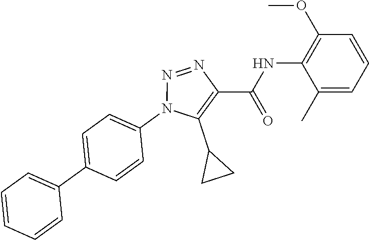 [SMILES: COc1cccc(C)c1NC(=O)c1nnn(-c2ccc(-c3ccccc3)cc2)c1C1CC1] | 14-62 | 1.142 | 424.5 | 425.3 | [M + H]⁺ | 5 |
| 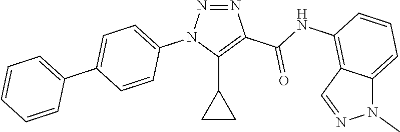 [SMILES: Cn1ncc2c(NC(=O)c3nnn(-c4ccc(-c5ccccc5)cc4)c3C3CC3)cccc21] | 14-63 | 0.988 | 434.5 | 435 | [M + H]⁺ | 4 |
| 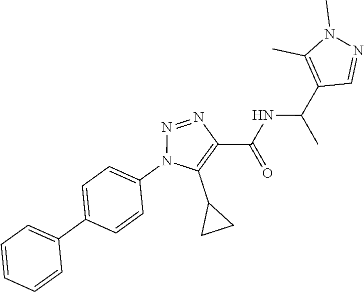 [SMILES: Cc1c(C(C)NC(=O)c2nnn(-c3ccc(-c4ccccc4)cc3)c2C2CC2)cnn1C] | 14-64 | 0.870 | 426.5 | 427.4 | [M + H]⁺ | 4 |
| 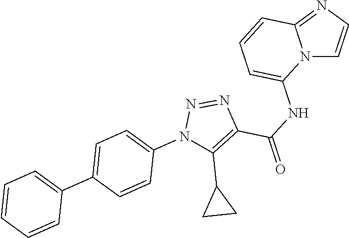 [SMILES: O=C(Nc1cccc2nccn12)c1nnn(-c2ccc(-c3ccccc3)cc2)c1C1CC1] | 14-65 | 1.032 | 420.5 | 421.3 | [M + H]⁺ | 5 |
| 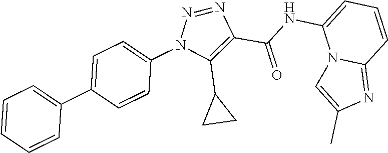 [SMILES: Cc1cn2c(NC(=O)c3nnn(-c4ccc(-c5ccccc5)cc4)c3C3CC3)cccc2n1] | 14-66 | 0.841 | 434.5 | 435 | [M + H]⁺ | 4 |
| 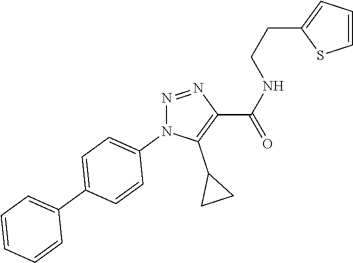 [SMILES: O=C(NCCc1cccs1)c1nnn(-c2ccc(-c3ccccc3)cc2)c1C1CC1] | 14-67 | 1.15 | 414.5 | 415.2 | [M + H]⁺ | 5 |
| 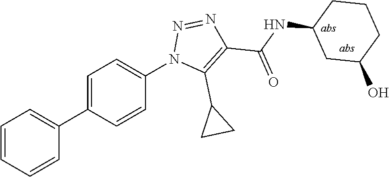 [SMILES: O=C(N[C@H]1CCC[C@@H](O)C1)c1nnn(-c2ccc(-c3ccccc3)cc2)c1C1CC1] | 14-68 | 0.902 | 402.5 | 403 | [M + H]⁺ | 4 |
| 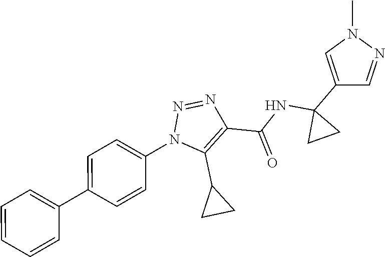 [SMILES: Cn1cc(C2(NC(=O)c3nnn(-c4ccc(-c5ccccc5)cc4)c3C3CC3)CC2)cn1] | 14-69 | 1.034 | 424.5 | 425.3 | [M + H]⁺ | 5 |
| 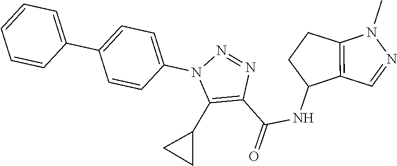 [SMILES: Cn1ncc2c1CCC2NC(=O)c1nnn(-c2ccc(-c3ccccc3)cc2)c1C1CC1] | 14-70 | 0.920 | 424.5 | 425.3 | [M + H]⁺ | 4 |
| 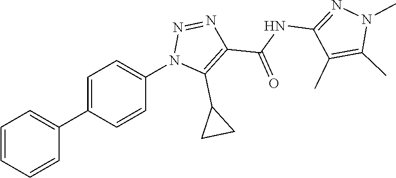 [SMILES: Cc1c(NC(=O)c2nnn(-c3ccc(-c4ccccc4)cc3)c2C2CC2)nn(C)c1C] | 14-71 | 0.942 | 412.5 | 413 | [M + H]⁺ | 4 |
| 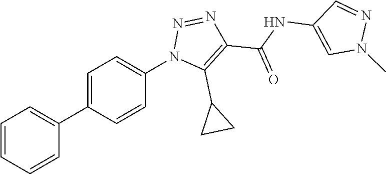 [SMILES: Cn1cc(NC(=O)c2nnn(-c3ccc(-c4ccccc4)cc3)c2C2CC2)cn1] | 14-72 | 1.023 | 384.4 | 385.2 | [M + H]⁺ | 5 |
| 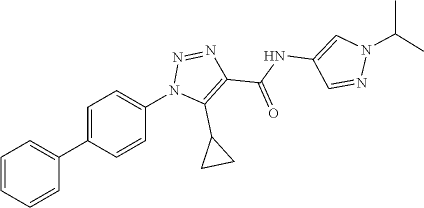 [SMILES: CC(C)n1cc(NC(=O)c2nnn(-c3ccc(-c4ccccc4)cc3)c2C2CC2)cn1] | 14-73 | 0.964 | 412.5 | 413.2 | [M + H]⁺ | 4 |
| 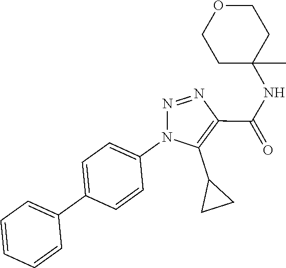 [SMILES: CC1(NC(=O)c2nnn(-c3ccc(-c4ccccc4)cc3)c2C2CC2)CCOCC1] | 14-74 | 1.086 | 402.5 | 403.3 | [M + H]⁺ | 5 |
| 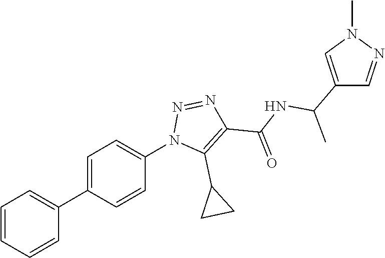 [SMILES: CC(NC(=O)c1nnn(-c2ccc(-c3ccccc3)cc2)c1C1CC1)c1cnn(C)c1] | 14-75 | 0.963 | 412.5 | 413 | [M + H]⁺ | 5 |
| 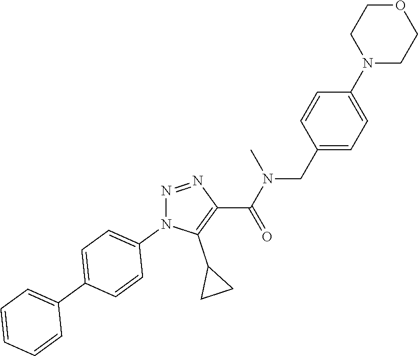 [SMILES: CN(Cc1ccc(N2CCOCC2)cc1)C(=O)c1nnn(-c2ccc(-c3ccccc3)cc2)c1C1CC1] | 14-76 | 1.026 | 493.6 | 987.5 | [M + H]⁺ | 5 |
| 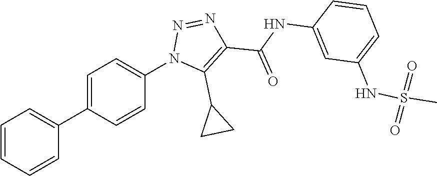 [SMILES: CS(=O)(=O)Nc1cccc(NC(=O)c2nnn(-c3ccc(-c4ccccc4)cc3)c2C2CC2)c1] | 14-77 | 0.996 | 473.6 | 474.1 | [M + H]⁺ | 4 |
| 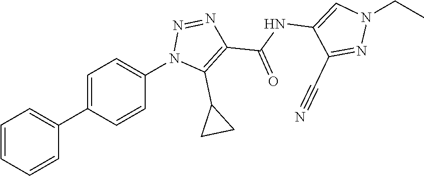 [SMILES: CCn1cc(NC(=O)c2nnn(-c3ccc(-c4ccccc4)cc3)c2C2CC2)c(C#N)n1] | 14-78 | 0.976 | 423.5 | 424.3 | [M + H]⁺ | 4 |
| 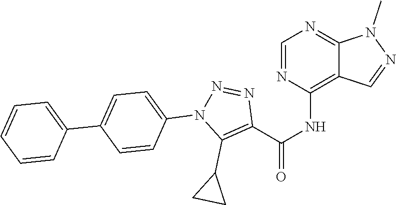 [SMILES: Cn1ncc2c(NC(=O)c3nnn(-c4ccc(-c5ccccc5)cc4)c3C3CC3)ncnc21] | 14-79 | 1 | 436.5 | 437.2 | [M + H]⁺ | 4 |
| 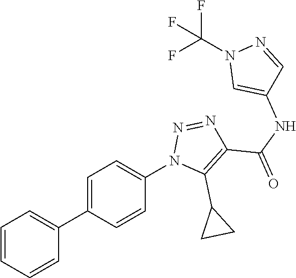 [SMILES: O=C(Nc1cnn(C(F)(F)F)c1)c1nnn(-c2ccc(-c3ccccc3)cc2)c1C1CC1] | 14-80 | 1.013 | 438.4 | 439.2 | [M + H]⁺ | 4 |
| 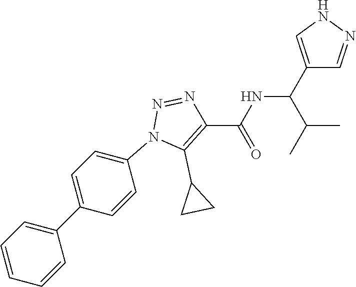 [SMILES: CC(C)C(NC(=O)c1nnn(-c2ccc(-c3ccccc3)cc2)c1C1CC1)c1cn[nH]c1] | 14-81 | 0.948 | 426.5 | 427 | [M + H]⁺ | 4 |
| 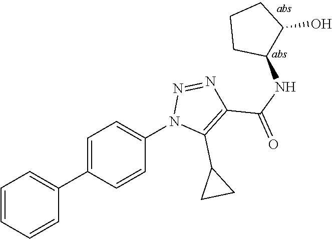 [SMILES: O=C(N[C@H]1CCC[C@@H]1O)c1nnn(-c2ccc(-c3ccccc3)cc2)c1C1CC1] | 14-82 | 0.913 | 388.5 | 389 | [M + H]⁺ | 4 |
| 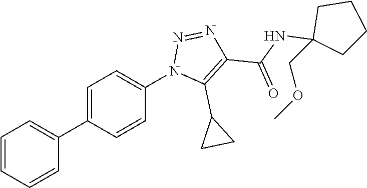 [SMILES: COCC1(NC(=O)c2nnn(-c3ccc(-c4ccccc4)cc3)c2C2CC2)CCCC1] | 14-83 | 1.165 | 416.5 | 417.3 | [M + H]⁺ | 4 |
| 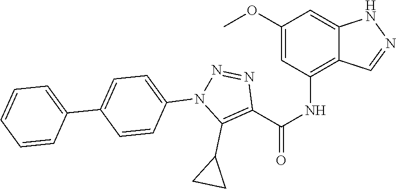 [SMILES: COc1cc(NC(=O)c2nnn(-c3ccc(-c4ccccc4)cc3)c2C2CC2)c2cn[nH]c2c1] | 14-84 | 0.990 | 450.5 | 451 | [M + H]⁺ | 4 |
| 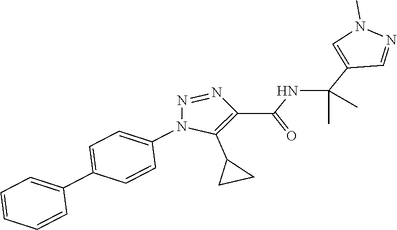 [SMILES: Cn1cc(C(C)(C)NC(=O)c2nnn(-c3ccc(-c4ccccc4)cc3)c2C2CC2)cn1] | 14-85 | 0.884 | 426.5 | 427.2 | [M + H]⁺ | 5 |
| 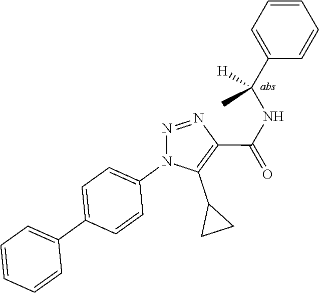 [SMILES: C[C@H](NC(=O)c1nnn(-c2ccc(-c3ccccc3)cc2)c1C1CC1)c1ccccc1] | 14-86 | 1.165 | 408.5 | 409.3 | [M + H]⁺ | 5 |
| 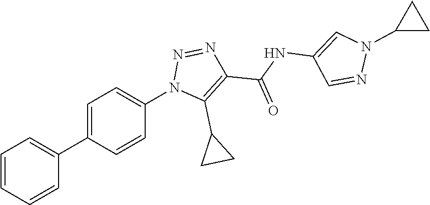 [SMILES: O=C(Nc1cnn(C2CC2)c1)c1nnn(-c2ccc(-c3ccccc3)cc2)c1C1CC1] | 14-87 | 0.947 | 410.5 | 411.4 | [M + H]⁺ | 4 |
| 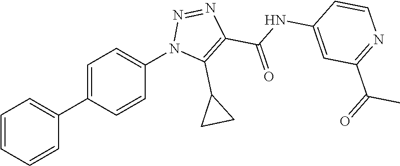 [SMILES: CC(=O)c1cc(NC(=O)c2nnn(-c3ccc(-c4ccccc4)cc3)c2C2CC2)ccn1] | 14-88 | 0.937 | 423.5 | 424.2 | [M + H]⁺ | 4 |

### Example 15

### Synthesis of Compound 15-4 and Other Representative Compounds

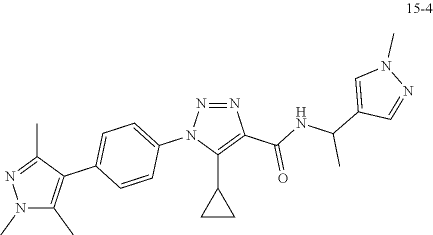 [SMILES: Cc1nn(C)c(C)c1-c1ccc(-n2nnc(C(=O)NC(C)c3cnn(C)c3)c2C2CC2)cc1]

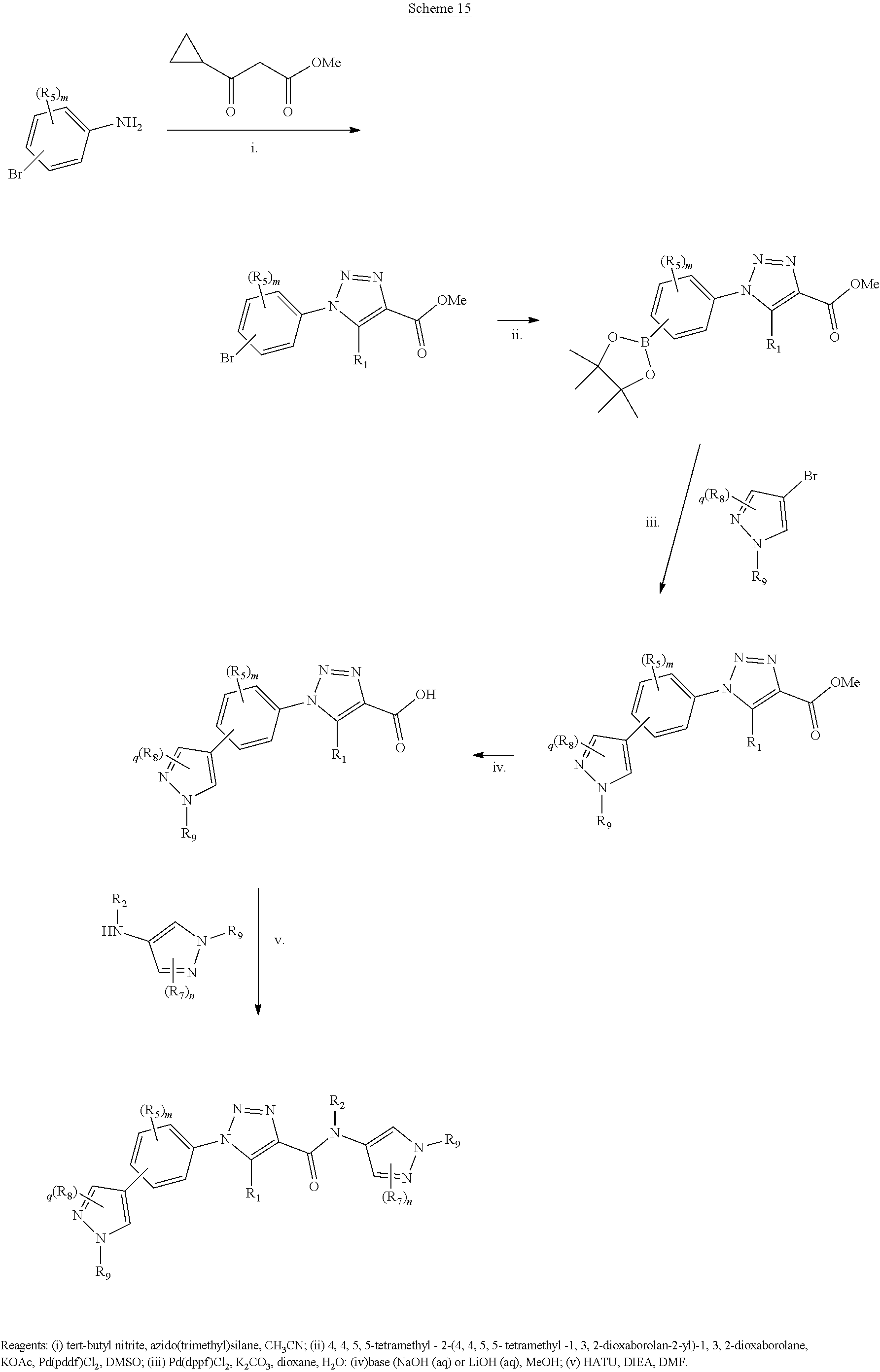

### Step 15-1. Synthesis of methyl 1-(4-bromophenyl)-5-cyclopropyl-1H-1,2,3-triazole-4-carboxylate (INT 15A)

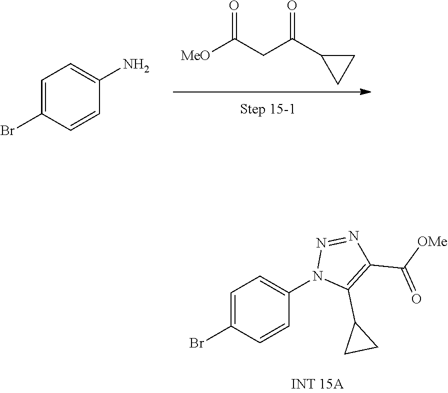 [SMILES: Nc1ccc(Br)cc1 ; COC(=O)CC(=O)C1CC1 ; COC(=O)c1nnn(-c2ccc(Br)cc2)c1C1CC1]

To a solution of 4-bromoaniline (10 g, 58.13 mmol) in CH₃CN (200 mL) were added tert-butyl nitrite (10.37 mL, 87.20 mmol) and azido (trimethyl)silane (9.17 mL, 69.76 mmol) at 0° C. After stirring at 20° C. for 2 h, methyl 3-cyclopropyl-3-oxo-propanoate (9.92 g, 69.76 mmol) and K₂CO₃ (24.10 g, 174.39 mmol) were added, and the mixture was further stirred at 40° C. for 15 h. The reaction mixture was poured into H₂O at 0° C. and extracted with EA. The combined organic layers were washed with brine, dried (Na₂SO₄), and concentrated to a residue that was triturated with ethanol at 15° C. to produce 8.5 g (45.3%) of methyl 3-(((2,4-dichlorophenyl)thio)methyl)benzoate (INT 15A) as a yellow solid. LCMS-ESI (m/z) calculated for C₁₃H₁₂BrN₃O₂: 321.0; found 321.9 [M+H]+, t_{R}=0.87 min (Method 4). ¹H NMR (400 MHz, CDCl₃) δ=7.77-7.67 (m, 2H), 7.50-7.41 (m, 2H), 3.99 (s, 3H), 1.95 (tt, J=5.5, 8.6 Hz, 1H), 1.08-1.02 (m, 2H), 0.88-0.82 (m, 2H).

### Step 15-2. Synthesis of methyl 5-cyclopropyl-1-(4-(4,4,5,5-tetramethyl-1,3,2-dioxaborolan-2-yl)phenyl)-1H-1,2,3-triazole-4-carboxylate (INT 15B)

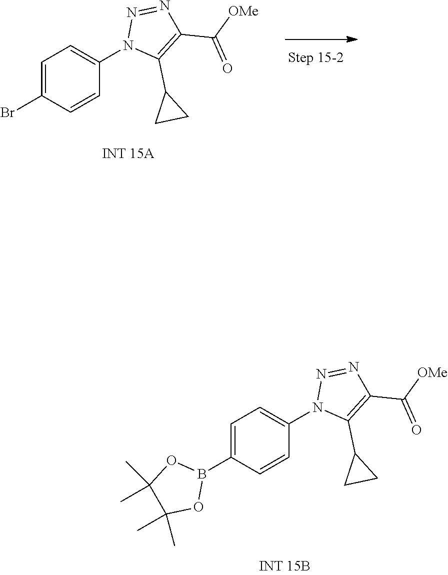 [SMILES: COC(=O)c1nnn(-c2ccc(Br)cc2)c1C1CC1 ; COC(=O)c1nnn(-c2ccc(B3OC(C)(C)C(C)(C)O3)cc2)c1C1CC1]

To a stirring solution of INT 15A (500 mg, 1.55 mmol) in DMSO (4 mL) were added 4,4,5,5-tetramethyl-2-(4,4,5,5-tetramethyl-1,3,2-dioxaborolan-2-yl)-1,3,2-dioxaborolane (600 mg, 2.36 mmol), KOAc (304.64 mg, 3.10 mmol) and Pd(dppf)Cl₂ (113.56 mg, 0.15 mmol). The reaction mixture was degassed and heated at 80° C. for 15 h under an atmosphere of N₂. The reaction mixture was diluted with H₂O and EA. The aqueous layer extracted with EA, and the organic layers were combined, washed with brine, dried (Na₂SO₄), filtered and concentrated to give 600 mg (99%) of crude methyl 5-cyclopropyl-1-(4-(4,4,5,5-tetramethyl-1,3,2-dioxaborolan-2-yl) phenyl)-1H-1,2,3-triazole-4-carboxylate (INT 15B) as a brown oil that was used in the next step without further purification. LCMS-ESI (m/z) calculated for C₁₉H₂₄BN₃O₄: 369.2; found 370.2 [M+Na]⁺, t_{R}=0.875 min(Method 5)

### Step 15-3. Synthesis of methyl 5-cyclopropyl-1-(4-(1,3,5-trimethyl-1H-pyrazol-4-yl)phenyl)-1H-1,2,3-triazole-4-carboxylate (INT 15C)

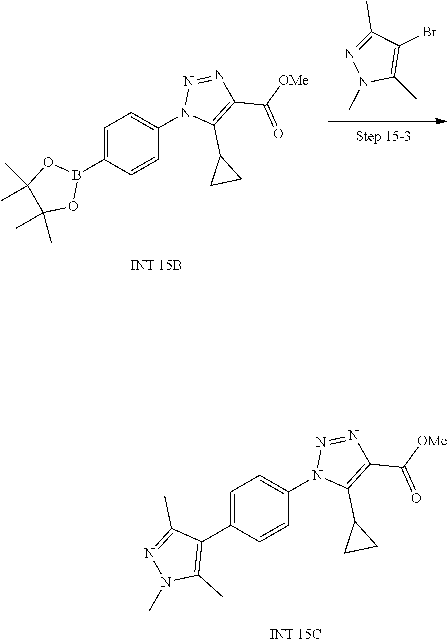

To a solution of INT 15B (600 mg, 1.63 mmol) in dioxane (10 mL) and H₂O (2 mL) were added 4-bromo-1,3,5-trimethyl-pyrazole (460.83 mg, 2.44 mmol), Pd(dppf)Cl₂ (118.91 mg, 0.162 mmol) and K₂CO₃ (449.18 mg, 3.25 mmol). The reaction mixture was degassed and stirred at 80° C. for 15 h under an atmosphere of N₂. The reaction mixture was concentrated to a residue that was purified by SiO₂ chromatography (EA/pet ether) to produce 240 mg (34.5%) of methyl 5-cyclopropyl-1-(4-(1,3,5-trimethyl-1H-pyrazol-4-yl)phenyl)-1H-1,2,3-triazole-4-carboxylate (INT 15C) as a yellow solid. LCMS-ESI (m/z) calculated for C₁₉H₂₁N₅O₂: 351.4; found 352.2 [M+H]⁺, t_{R}=0.76 min (Method 4). ¹H NMR (400 MHz, CDCl₃) δ=7.57 (d, J=8.4 Hz, 2H), 7.42 (d, J=8.4 Hz, 2H), 3.99 (s, 3H), 3.81 (s, 3H), 2.29 (d, J=1.5 Hz, 6H), 2.05-2.00 (m, 1H), 1.09-1.02 (m, 2H), 0.99-0.91 (m, 2H).

### Step 15-4. Synthesis of 5-cyclopropyl-1-(4-(1,3,5-trimethyl-1H-pyrazol-4-yl)phenyl)-1H-1,2,3-triazole-4-carboxylic acid (INT 15-D)

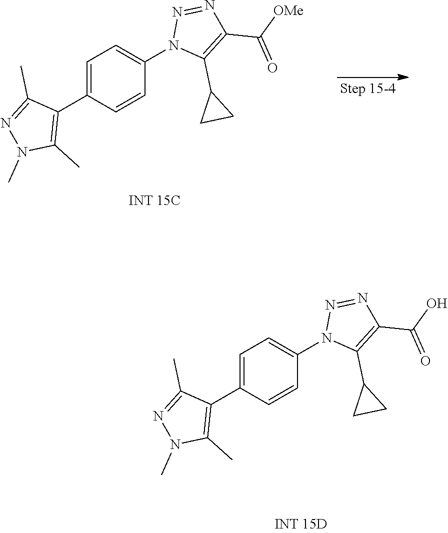 [SMILES: COC(=O)c1nnn(-c2ccc(-c3c(C)nn(C)c3C)cc2)c1C1CC1 ; Cc1nn(C)c(C)c1-c1ccc(-n2nnc(C(=O)O)c2C2CC2)cc1]

To a solution of INT 15C (240 mg, 683 μmol) in MeOH (20 mL) was added LiOH (1 M, 2 mL, 2 mmol). The mixture was stirred at 15° C. for 16 h then concentrated to remove MeOH. The resulting aqueous solution was pH adjusted to pH=7 using 1M HCl, creating a yellow precipitate that was collected by filtration and dried under vacuum to provide 200 mg (84%) of 5-cyclopropyl-1-(4-(1,3,5-trimethyl-1H-pyrazol-4-yl)phenyl)-1H-1,2,3-triazole-4-carboxylic acid (INT 15D) as a yellow solid. LCMS-ESI (m/z) calculated for C₁₉H₁₉N₅O₂: 337.2; found 338.2 [M+H]⁺, t_{R}=0.2 min (Method 5). ¹H NMR (400 MHz, DMSO-D₆) δ=13.53-12.45 (s, 1H), 7.69 (br d, J=8.1 Hz, 2H), 7.49 (br d, J=8.1 Hz, 2H), 3.73 (s, 3H), 2.39-2.16 (m, 6H), 2.14-2.05 (m, 1H), 0.98-0.83 (m, 2H), 0.73 (br d, J=3.9 Hz, 2H).

### Step 15-5. Synthesis of 5-cyclopropyl-N-(1-(1-methyl-1H-pyrazol-4-yl)ethyl)-1-(4-(1,3,5-trimethyl-1H-pyrazol-4-yl)phenyl)-1H-1,2,3-triazole-4-carboxamide (Compound 15-41

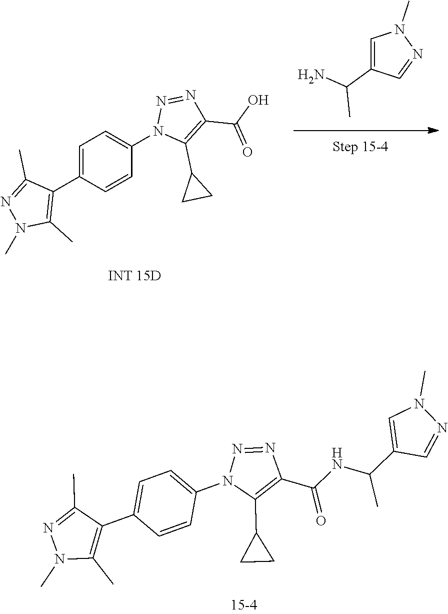

To a stirring mixture of INT 15D (50 mg, 148.2 μmol) and 1-(1-methyl pyrazol-4-yl) ethanamine (27.83 mg, 222.3 μmol) in DMF (3 mL) were added HATU (112.7 mg, 296.4 μmol) and DIEA (57.46 mg, 77.44 μL, 444.61 μmol). After stirring at 15° C. for 1 h, the mixture was diluted with saturated, aqueous, NaHCO₃ and extracted with EA (2×). The combined organic layers were washed, dried (Na₂SO₄), concentrated, and purified by prep HPLC (10 mM aqueous NH₄CO₃/CH₃CN) to produce 32.6 mg (49.5%) of 5-cyclopropyl-N-(1-(1-methyl-1H-pyrazol-4-yl) ethyl)-1-(4-(1,3,5-trimethyl-1H-pyrazol-4-yl) phenyl)-1H-1,2,3-triazole-4-carboxamide (Compound 15-4) as a yellow solid. LCMS-ESI (m/z) calculated for C₂₄H₂₈N₈O: 444.5; found 445.2 [M+H]⁺, t_{R}=0.84 min (Method 4). ¹H NMR (400 MHz, CDCl₃) δ=7.58-7.53 (m, 2H), 7.50 (s, 1H), 7.48-7.40 (m, 2H), 7.38 (s, 1H), 5.41-5.23 (m, 1H), 3.89 (s, 3H), 3.82 (s, 3H), 2.30 (d, J=1.5 Hz, 6H), 2.06-1.95 (m, 1H), 1.62-1.62 (m, 1H), 1.60 (d, J=6.9 Hz, 3H), 1.21-1.12 (m, 2H), 1.08-1.00 (m, 2H).

The compounds listed in the Table 15 were made using the procedures of Scheme 15.

TABLE 15

| | | Purity | | | | |
|---|---|---|---|---|---|---|
| | Cpd | RT | | Observed | | Purity |
| Structure | No. | (min) | MW | m/z | Ion | Method |
| 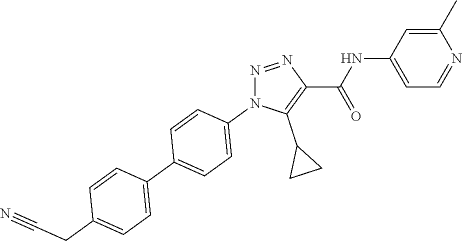 [SMILES: Cc1cc(NC(=O)c2nnn(-c3ccc(-c4ccc(CC#N)cc4)cc3)c2C2CC2)ccn1] | 15-1 | 0.427 | 434.5 | 435.2 | [M + H]⁺ | 7 |
| 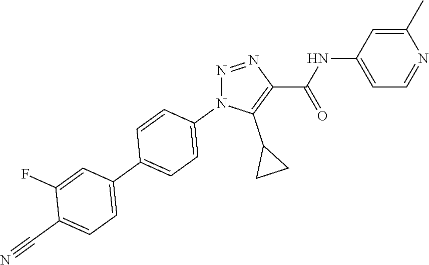 [SMILES: Cc1cc(NC(=O)c2nnn(-c3ccc(-c4ccc(C#N)c(F)c4)cc3)c2C2CC2)ccn1] | 15-2 | 0.469 | 438.5 | 439.3 | [M + H]⁺ | 7 |
| 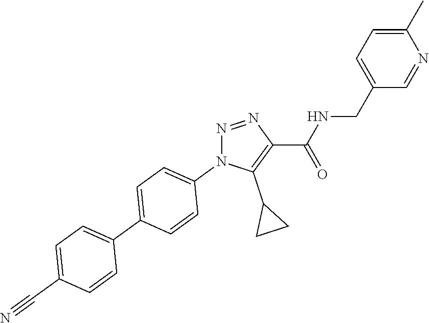 [SMILES: Cc1ccc(CNC(=O)c2nnn(-c3ccc(-c4ccc(C#N)cc4)cc3)c2C2CC2)cn1] | 15-3 | 0.461 | 434.5 | 435.4 | [M + H]⁺ | 7 |
| 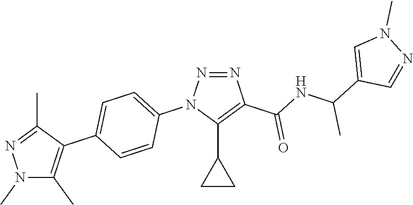 [SMILES: Cc1nn(C)c(C)c1-c1ccc(-n2nnc(C(=O)NC(C)c3cnn(C)c3)c2C2CC2)cc1] | 15-4 | 0.843 | 444.5 | 445.2 | [M + H]⁺ | 4 |
| 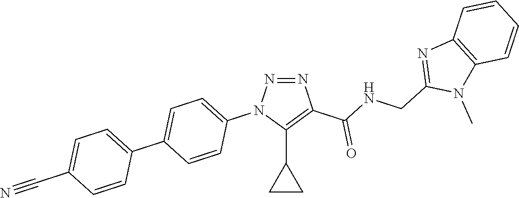 [SMILES: Cn1c(CNC(=O)c2nnn(-c3ccc(-c4ccc(C#N)cc4)cc3)c2C2CC2)nc2ccccc21] | 15-5 | 0.818 | 473.5 | 474 | [M + H]⁺ | 4 |
| 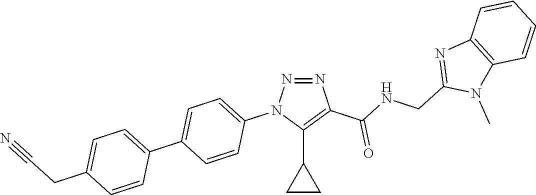 [SMILES: Cn1c(CNC(=O)c2nnn(-c3ccc(-c4ccc(CC#N)cc4)cc3)c2C2CC2)nc2ccccc21] | 15-6 | 0.817 | 487.6 | 488 | [M + H]⁺ | 4 |
| 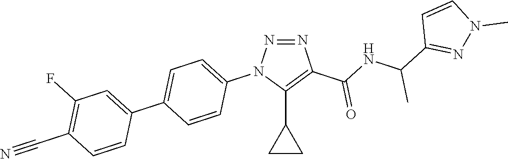 [SMILES: CC(NC(=O)c1nnn(-c2ccc(-c3ccc(C#N)c(F)c3)cc2)c1C1CC1)c1ccn(C)n1] | 15-7 | 0.829 | 455.5 | 456.2 | [M + H]⁺ | 4 |
| 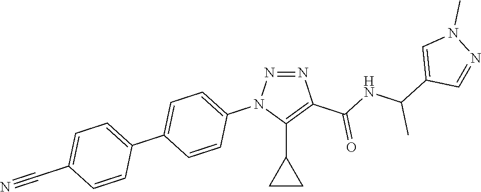 [SMILES: CC(NC(=O)c1nnn(-c2ccc(-c3ccc(C#N)cc3)cc2)c1C1CC1)c1cnn(C)c1] | 15-8 | 0.917 | 437.5 | 438.2 | [M + H]⁺ | 4 |
| 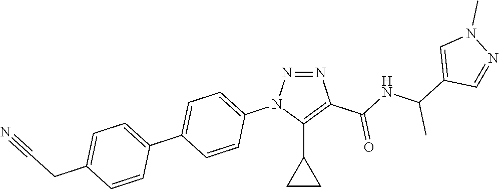 [SMILES: CC(NC(=O)c1nnn(-c2ccc(-c3ccc(CC#N)cc3)cc2)c1C1CC1)c1cnn(C)c1] | 15-9 | 0.930 | 451.5 | 452.2 | [M + H]⁺ | 4 |
| 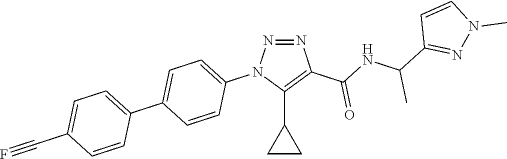 | 15-10 | 0.828 | 437.5 | 438.2 | [M + H]⁺ | 4 |
| 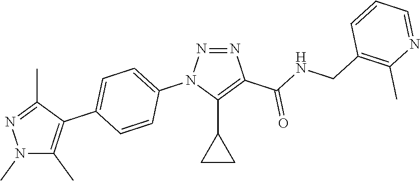 [SMILES: Cc1ncccc1CNC(=O)c1nnn(-c2ccc(-c3c(C)nn(C)c3C)cc2)c1C1CC1] | 15-11 | 0.723 | 441.5 | 442.2 | [M + H]⁺ | 4 |
| 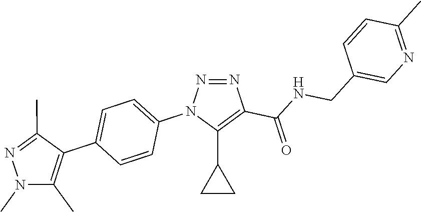 | 15-12 | 0.478 | 441.5 | 442.1 | [M + H]⁺ | 4 |
| 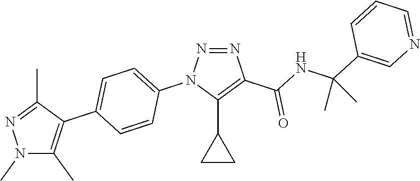 [SMILES: Cc1nn(C)c(C)c1-c1ccc(-n2nnc(C(=O)NC(C)(C)c3cccnc3)c2C2CC2)cc1] | 15-13 | 0.805 | 455.6 | 456.2 | [M + H]⁺ | 5 |
| 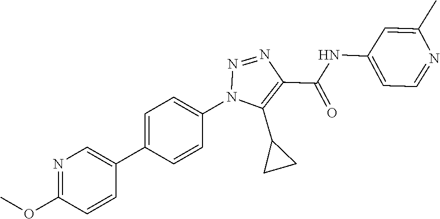 | 15-14 | 0.861 | 426.5 | 427.1 | [M + H]⁺ | 5 |
| 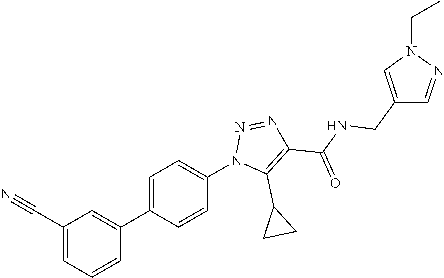 [SMILES: CCn1cc(CNC(=O)c2nnn(-c3ccc(-c4cccc(C#N)c4)cc3)c2C2CC2)cn1] | 15-15 | 0.888 | 437.5 | 438.3 | [M + H]⁺ | 4 |
| 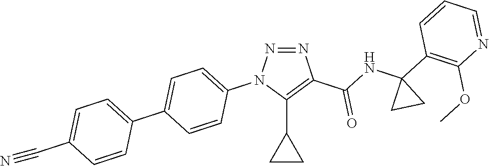 [SMILES: COc1ncccc1C1(NC(=O)c2nnn(-c3ccc(-c4ccc(C#N)cc4)cc3)c2C2CC2)CC1] | 15-16 | 0.962 | 476.5 | 477.3 | [M + H]⁺ | 4 |
| 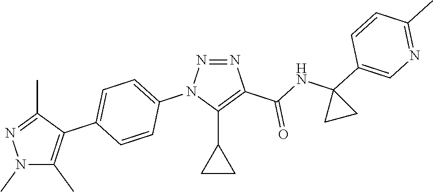 | 15-17 | 0.766 | 467.6 | 468 | [M + H]⁺ | 4 |
| 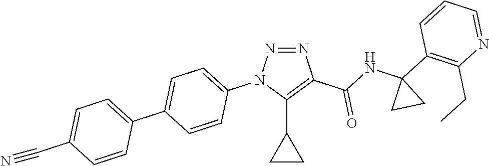 [SMILES: CCc1ncccc1C1(NC(=O)c2nnn(-c3ccc(-c4ccc(C#N)cc4)cc3)c2C2CC2)CC1] | 15-18 | 0.817 | 474.6 | 475.4 | [M + H]⁺ | 4 |
| 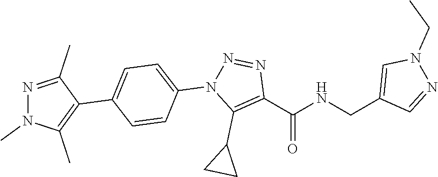 [SMILES: CCn1cc(CNC(=O)c2nnn(-c3ccc(-c4c(C)nn(C)c4C)cc3)c2C2CC2)cn1] | 15-19 | 0.835 | 444.5 | 445.3 | [M + H]⁺ | 4 |
| 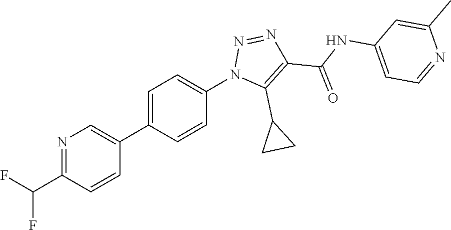 [SMILES: Cc1cc(NC(=O)c2nnn(-c3ccc(-c4ccc(C(F)F)nc4)cc3)c2C2CC2)ccn1] | 15-20 | 0.805 | 446.5 | 447 | [M + H]⁺ | 4 |
| 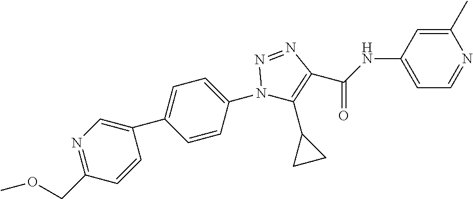 | 15-21 | 0.736 | 440.5 | 441 | [M + H]⁺ | 4 |
| 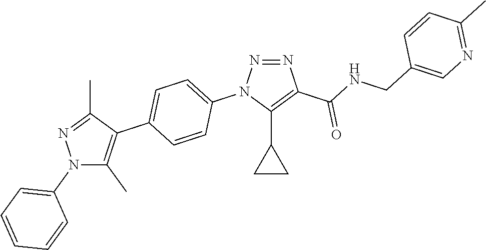 [SMILES: Cc1ccc(CNC(=O)c2nnn(-c3ccc(-c4c(C)nn(-c5ccccc5)c4C)cc3)c2C2CC2)cn1] | 15-22 | 0.829 | 503.6 | 504.4 | [M + H]⁺ | 4 |

### Example 16

### Synthesis of Compound 16-1 and

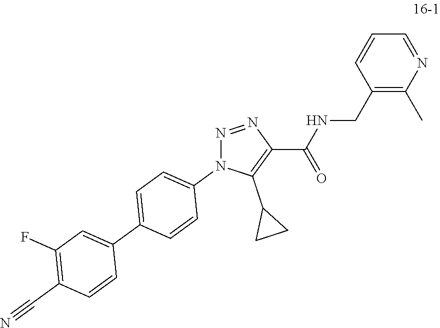 [SMILES: Cc1ncccc1CNC(=O)c1nnn(-c2ccc(-c3ccc(C#N)c(F)c3)cc2)c1C1CC1]

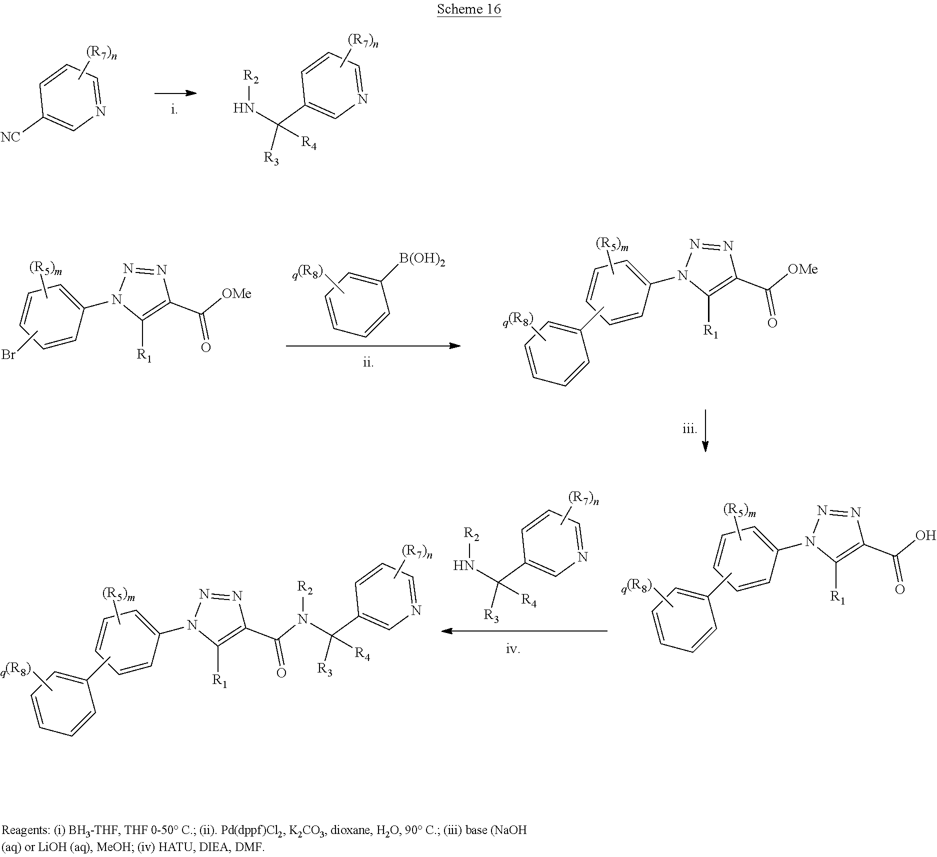

### Step 16-1. Synthesis of (2-methylpyridin-3-yl)methanamine (INT 16A)

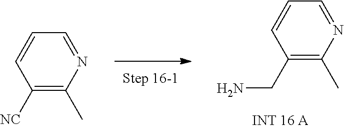

To the solution of 2-methylpyridine-3-carbonitrile (2 g, 16.93 mmol) in THF (10 mL) was added BH₃-THF (1 M, 40.00 mL, 40 mmol) at 0′° C. The resulting solution was heated to 50° C. for 4 h. The mixture was quenched by 1M HCl (10 mL) at 0° C. The solution was basified with aqueous NaOH solution to pH>10. The mixture was extracted with EA (3×). The combined organic extracts were dried (Na₂SO₄), filtered, and concentrated under reduced pressure to give a residue that was purified by reverse-phase HPLC chromatography (0.10/% NH₃·H₂O) to afford 980 mg (45.5%) of (2-methylpyridin-3-yl)methanamine (INT 16A). LCMS-ESI (m/z) calculated for C₇H₁₀N₂: 122.2; found 123.2 [M+H]⁺, t_{R}=0.25 min (Method 5). ¹H NMR (400 MHz, CDCl₃) δ 8.41-8.32 (m, 1H), 7.64 (br d, J=7.7 Hz, 1H), 7.17-7.11 (m, 1H), 3.89 (d, J=4.1 Hz, 2H), 2.55 (br d, J=7.5 Hz, 3H).

### Step 16-2. Synthesis of methyl 1-(4′-cyano-3′-fluoro-[1,1′-biphenyl]-4-yl)-5-cyclopropyl-1H-1,2,3-triazole-4-carboxylate (16 B)

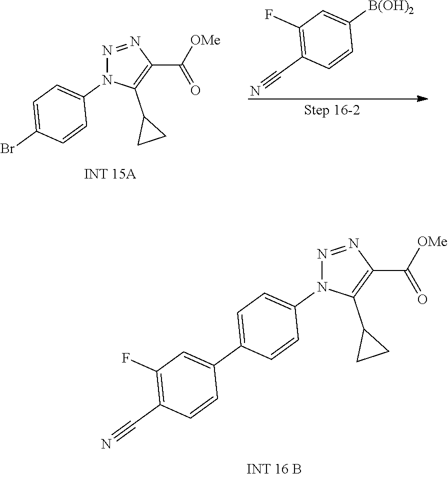 [SMILES: COC(=O)c1nnn(-c2ccc(Br)cc2)c1C1CC1 ; N#Cc1ccc(B(O)O)cc1F ; COC(=O)c1nnn(-c2ccc(-c3ccc(C#N)c(F)c3)cc2)c1C1CC1]

Into a mixture of (4-cyano-3-fluoro-phenyl)boronic acid (550 mg, 3.33 mmol) in dioxane (12 mL) and H₂O (4 mL), were added INT 15A (1 g, 3.10 mmol), Pd(dppf)Cl₂ (227.13 mg, 310.41 μmol) and K₂CO₃ (858 mg, 6.21 mmol). The solution was de-gassed and then heated to 90° C. for 15 hours under N₂. The reaction mixture was concentrated to a residue that was purified by SiO₂ chromatography (EA/petroleum ether) to afford 900 mg (76%) methyl 1-(4′-cyano-3′-fluoro-[1,1′-biphenyl]-4-yl)-5-cyclopropyl-1H-1,2,3-triazole-4-carboxylate (INT 16B) as a yellow solid. LCMS-ESI (m/z) calculated for C₂₀H₁₅FN₄O₂: 362.3: found 363.2 [M+H]⁺, t_{R}=0.917 min (Method 4).

### Step 16-3. Synthesis of 1-(4′-cyano-3′-fluoro-[1,1′-biphenyl]-4-yl)-5-cyclopropyl-1H-1,2,3-triazole-4-carboxylic acid (INT 16C)

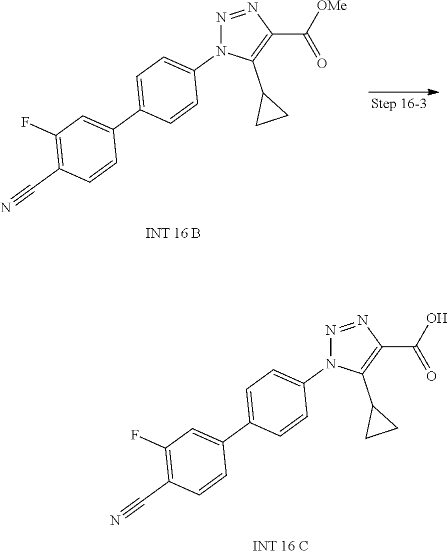 [SMILES: COC(=O)c1nnn(-c2ccc(-c3ccc(C#N)c(F)c3)cc2)c1C1CC1 ; N#Cc1ccc(-c2ccc(-n3nnc(C(=O)O)c3C3CC3)cc2)cc1F]

To a solution of INT 16B (900 mg, 2.48 mmol) in MeOH (20 mL) was added LiOH (1 M, 6 mL, 6 mmol). The reaction mixture was stirred at 25° C. for 15 h and then concentrated to remove MeOH. The resulting aqueous residue was pH adjusted to pH=7 with 1M HCl, creating a yellow precipitate that was collected by filtration to afford 900 mg of crude 1-(4′-cyano-3′-fluoro-[1,1′-biphenyl]-4-yl)-5-cyclopropyl-1H-1,2,3-triazole-4-carboxylic acid (INT 16C) as a yellow solid (72% purity) that was used without further purification. LCMS-ESI (m/z) calculated for C₁₉H₁₃FN₄O₂: 348.3; found 349.2 [M+H]⁺, t_{R}=0.873 min (Method 4). ¹H NMR (400 MHz, DMSO-D₆) δ=8.15-7.95 (m, 4H), 7.88 (dd, J=1.4, 8.2 Hz, 1H), 7.80 (d, J=8.5 Hz, 2H), 2.12-1.98 (m, 1H), 0.90-0.80 (m, 4H).

### Step 16-4. Synthesis of 1-(4′-cyano-3′-fluoro-[1,1′-biphenyl]-4-yl)-5-cyclopropyl-N-((2-methylpyridin-3-yl)methyl)-1H-1,2,3-triazole-4-carboxamide (Compound 16-1)

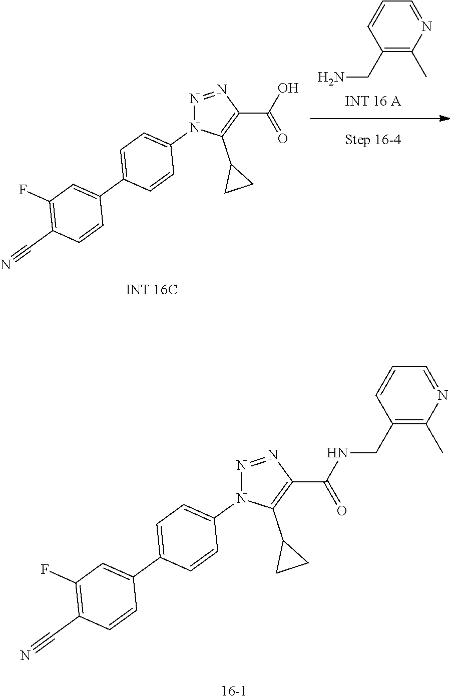

To a mixture of INT 16C (100 mg, 206.7 μmol, z purity) and TNT 16A (37.9 mg, 310.1 μmol) in DMF (4 mL) were added HATU (157.2 mg, 413.4 μmol) and DIEA (108 μL, 620.1 μmol). After stirring at 25° C. for 1 h, the mixture was concentrated to give a residue that was diluted with TEA (2 mL) and CH₃CN (10 mL). The mixture was stirred at 25° C. for 10 min and concentrated in vacuo to give a residue that was purified by prep-HPLC (aqueous NH₄HCO₃/CH₃CN) and then prep HPLC (H₂O (FA)/CH₃CN) to provide 319 mg (33.5%) of 1-(4′-cyano-3′-fluoro-[1,1′-biphenyl]-4-yl)-5-cyclopropyl-N-((2-methylpyridin-3-yl) methyl)-1H-1,2,3-triazole-4-carboxamide (Compound 16-1) as a yellow solid. LCMS-ESI (m/z) calculated for C₂₆H₂₁FN₆O: 452.5; found 453.0 [M+H]⁺, t_{R}=0.969 min (Method 4). ¹H NMR (400 MHz, DMSO-D₆) δ=9.12 (t, J=6.1 Hz, 1H), 8.36-8.26 (m, 1H), 8.10-8.04 (m, 3H), 8.03 (d, J=1.5 Hz, 1H), 7.88 (dd, J=1.6, 8.1 Hz, 1H), 7.86-7.80 (m, 2H), 7.63-7.55 (m, 1H), 7.20 (dd, J=4.9, 7.6 Hz, 1H), 4.47 (d, J=6.0 Hz, 2H), 2.53 (s, 3H), 2.18-1.97 (m, 1H), 0.95-0.72 (m, 4H).

The compounds listed in Table 16 were made using the procedures of Scheme 16.

TABLE 16

| | | Purity | | | | |
|---|---|---|---|---|---|---|
| | Cpd | RT | | observed | | Purity |
| Structure | No. | (min) | MW | m/z | Ion | Method |
| 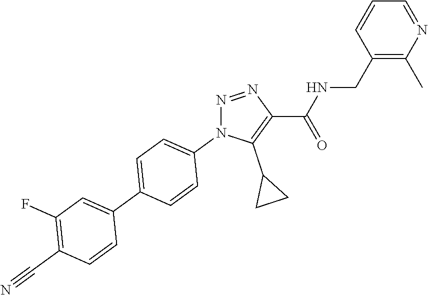 [SMILES: Cc1ncccc1CNC(=O)c1nnn(-c2ccc(-c3ccc(C#N)c(F)c3)cc2)c1C1CC1] | 16-1 | 0.812 | 452.5 | 453 | [M + H]⁺ | 4 |
| 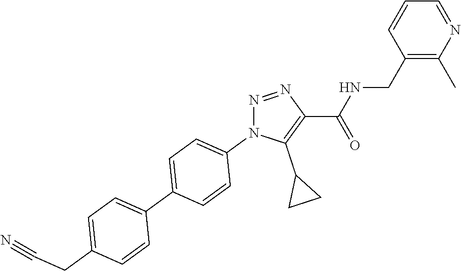 [SMILES: Cc1ncccc1CNC(=O)c1nnn(-c2ccc(-c3ccc(CC#N)cc3)cc2)c1C1CC1] | 16-2 | 0.939 | 448.5 | 449.1 | [M + H]⁺ | 5 |
| 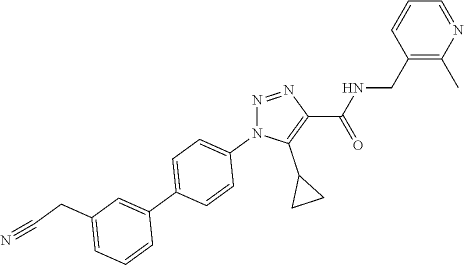 [SMILES: Cc1ncccc1CNC(=O)c1nnn(-c2ccc(-c3cccc(CC#N)c3)cc2)c1C1CC1] | 16-3 | 0.802 | 448.5 | 449 | [M + H]⁺ | 4 |
| 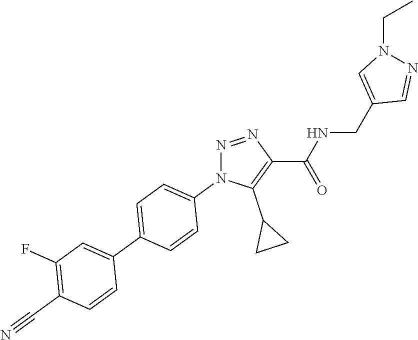 [SMILES: CCn1cc(CNC(=O)c2nnn(-c3ccc(-c4ccc(C#N)c(F)c4)cc3)c2C2CC2)cn1] | 16-4 | 0.906 | 455.5 | 456.3 | [M + H]⁺ | 4 |
| 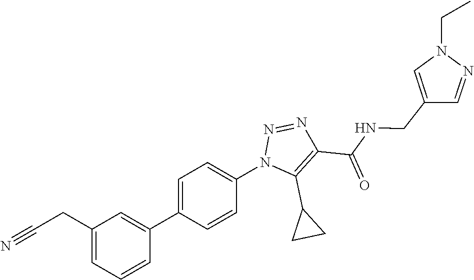 [SMILES: CCn1cc(CNC(=O)c2nnn(-c3ccc(-c4cccc(CC#N)c4)cc3)c2C2CC2)cn1] | 16-5 | 0.891 | 451.5 | 452.3 | [M + H]⁺ | 4 |

### Example 17

### Synthesis of Compound 17-3 and Other Representative Compounds

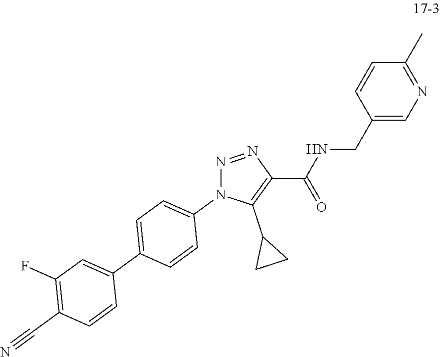 [SMILES: Cc1ccc(CNC(=O)c2nnn(-c3ccc(-c4ccc(C#N)c(F)c4)cc3)c2C2CC2)cn1]

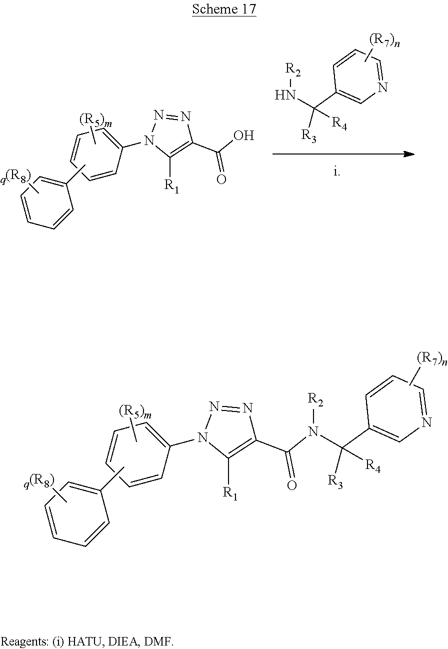

### Step 17-1. Synthesis of 1-(4′-cyano-3′-fluoro-[1,1′-biphenyl]-4-1)-5-cyclopropyl-N-((6-methylpyridin-3-yl)methyl)-1H-1,2,3-triazole-4-carboxamide (Compound 17-3)

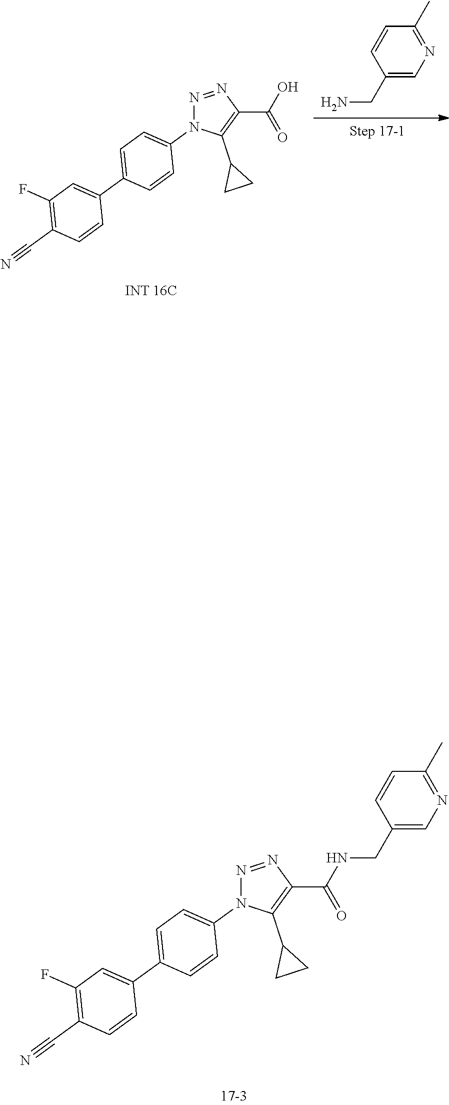

To a solution of INT 16C (60 mg, 172 μmol) and (6-methyl-3-pyridyl) methanamine (31.57 mg, 258.38 μmol) in DMF (3 mL) were added HATU (131 mg, 344 μmol) and DIEA (90 μL, 258.4 μmol). After stirring at 25° C. for 1 h, the mixture was concentrated to give a residue that was diluted with TEA (2 mL) and CH₃CN (10 mL). The mixture was stirred at 25° C. for 10 min and concentrated in vacuo to give a residue that was purified by prep-HPLC (aqueous NH₄HCO₃/CH₃CN) and then prep HPLC (H₂O (FA)/CH₃CN) to provide 31.7 mg (39.8%) of 1-(4′-cyano-3′-fluoro-[1,1′-biphenyl]-4-yl)-5-cyclopropyl-N-((6-methylpyridin-3-yl)methyl)-1H-1,2,3-triazole-4-carboxamide (Compound 17-3) as a yellow solid. LCMS-ESI (m/z) calculated for C₂₆H₂₁FN₆O: 452.5; found 453.0 [M+H]⁺, t_{R}=0.811 min(Method 4). ¹H NMR (400 MHz, DMSO-D₆) δ=9.15 (t, J=6.2 Hz, 1H), 8.42 (d, J=1.8 Hz, 1H), 8.13-8.00 (m, 4H), 7.88 (dd, J=1.5, 8.2 Hz, 1H), 7.83 (d, J=8.6 Hz, 2H), 7.63 (dd, J=2.2, 7.9 Hz, 1H), 7.21 (d, J=7.9 Hz, 11H), 4.44 (d, J=6.2 Hz, 2H), 2.44 (s, 3H₁), 2.12-2.01 (m, 1H), 0.91-0.81 (m, 4H).

The compounds listed in Table 17 were made using the procedures of Scheme 17.

TABLE 17

| | | Purity | | | | |
|---|---|---|---|---|---|---|
| | Cpd | RT | | observed | | Purity |
| Structure | No. | (min) | MW | m/z | Ion | Method |
| 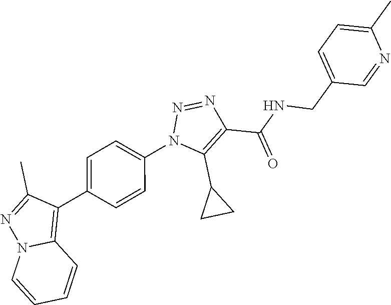 [SMILES: Cc1ccc(CNC(=O)c2nnn(-c3ccc(-c4c(C)nn5ccccc45)cc3)c2C2CC2)cn1] | 17-1 | 0.437 | 463.5 | 464.1 | [M + H]⁺ | 4 |
| 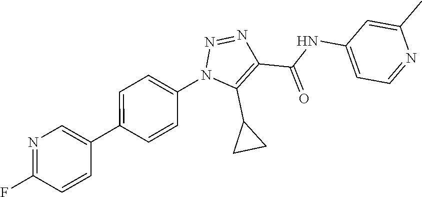 [SMILES: Cc1cc(NC(=O)c2nnn(-c3ccc(-c4ccc(F)nc4)cc3)c2C2CC2)ccn1] | 17-2 | 0.739 | 414.4 | 415.2 | [M + H]⁺ | 4 |
| 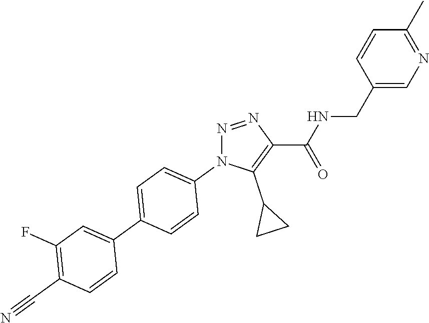 [SMILES: Cc1ccc(CNC(=O)c2nnn(-c3ccc(-c4ccc(C#N)c(F)c4)cc3)c2C2CC2)cn1] | 17-3 | 0.812 | 452.5 | 453 | [M + H]⁺ | 4 |
| 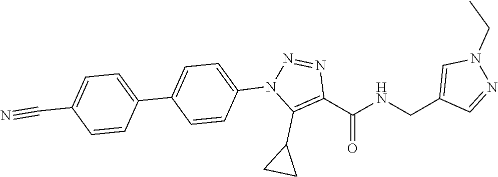 [SMILES: CCn1cc(CNC(=O)c2nnn(-c3ccc(-c4ccc(C#N)cc4)cc3)c2C2CC2)cn1] | 17-4 | 0.891 | 437.5 | 438.3 | [M + H]⁺ | 4 |
| 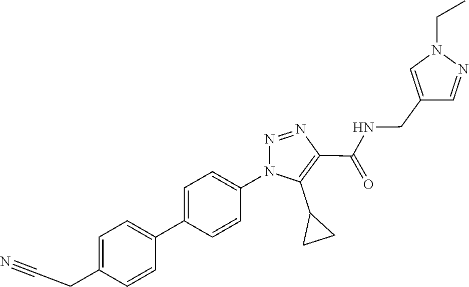 [SMILES: CCn1cc(CNC(=O)c2nnn(-c3ccc(-c4ccc(CC#N)cc4)cc3)c2C2CC2)cn1] | 17-5 | 0.890 | 451.5 | 452.3 | [M + H]⁺ | 4 |
| 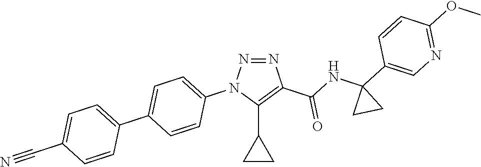 [SMILES: COc1ccc(C2(NC(=O)c3nnn(-c4ccc(-c5ccc(C#N)cc5)cc4)c3C3CC3)CC2)cn1] | 17-6 | 0.914 | 476.5 | 477.3 | [M + H]⁺ | 4 |
| 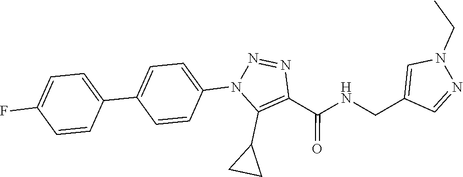 [SMILES: CCn1cc(CNC(=O)c2nnn(-c3ccc(-c4ccc(F)cc4)cc3)c2C2CC2)cn1] | 17-7 | 0.904 | 430.5 | 431.1 | [M + H]⁺ | 4 |
| 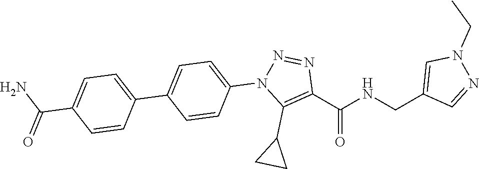 [SMILES: CCn1cc(CNC(=O)c2nnn(-c3ccc(-c4ccc(C(N)=O)cc4)cc3)c2C2CC2)cn1] | 17-8 | 0.829 | 455.5 | 456.3 | [M + H]⁺ | 4 |
| 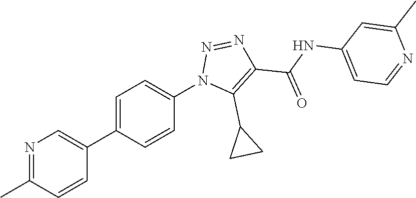 | 17-9 | 0.818 | 410.5 | 411.1 | [M + H]⁺ | 5 |
| 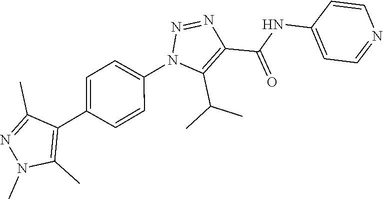 [SMILES: Cc1nn(C)c(C)c1-c1ccc(-n2nnc(C(=O)Nc3ccncc3)c2C(C)C)cc1] | 17-10 | 0.952 | 415.5 | 416.1 | [M + H]⁺ | 5 |
| 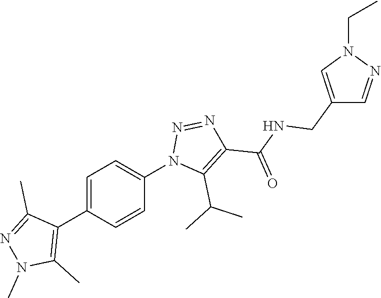 [SMILES: CCn1cc(CNC(=O)c2nnn(-c3ccc(-c4c(C)nn(C)c4C)cc3)c2C(C)C)cn1] | 17-11 | 0.879 | 446.6 | 447 | [M + H]⁺ | 4 |
| 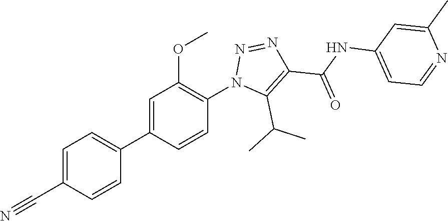 [SMILES: COc1cc(-c2ccc(C#N)cc2)ccc1-n1nnc(C(=O)Nc2ccnc(C)c2)c1C(C)C] | 17-12 | 0.455 | 452.5 | 453.2 | [M + H]⁺ | 4 |
| 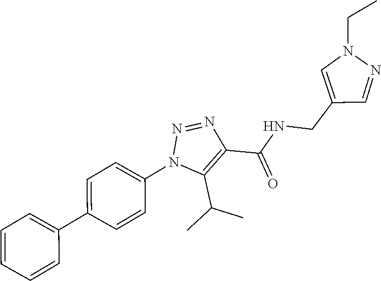 [SMILES: CCn1cc(CNC(=O)c2nnn(-c3ccc(-c4ccccc4)cc3)c2C(C)C)cn1] | 17-13 | 1.001 | 414.5 | 415.2 | [M + H]⁺ | 5 |
| 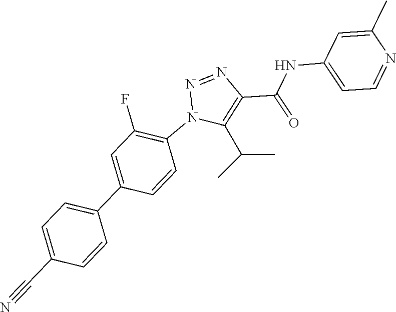 [SMILES: Cc1cc(NC(=O)c2nnn(-c3ccc(-c4ccc(C#N)cc4)cc3F)c2C(C)C)ccn1] | 17-14 | 0.463 | 440.5 | 441.3 | [M + H]⁺ | 4 |
| 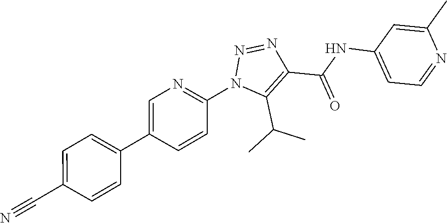 [SMILES: Cc1cc(NC(=O)c2nnn(-c3ccc(-c4ccc(C#N)cc4)cn3)c2C(C)C)ccn1] | 17-15 | 0.454 | 423.5 | 424.3 | [M + H]⁺ | 4 |
| 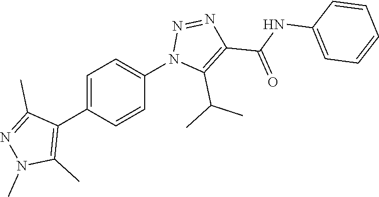 [SMILES: Cc1nn(C)c(C)c1-c1ccc(-n2nnc(C(=O)Nc3ccccc3)c2C(C)C)cc1] | 17-16 | 1.026 | 414.5 | 415.2 | [M + H]⁺ | 5 |
| 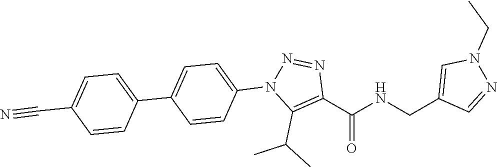 [SMILES: CCn1cc(CNC(=O)c2nnn(-c3ccc(-c4ccc(C#N)cc4)cc3)c2C(C)C)cn1] | 17-17 | 0.928 | 439.5 | 440.3 | [M + H]⁺ | 4 |
| 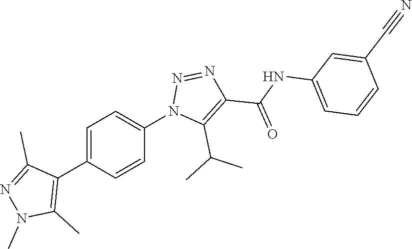 [SMILES: Cc1nn(C)c(C)c1-c1ccc(-n2nnc(C(=O)Nc3cccc(C#N)c3)c2C(C)C)cc1] | 17-18 | 9.705 | 439.5 | 440 | [M + H]⁺ | 1 |
| 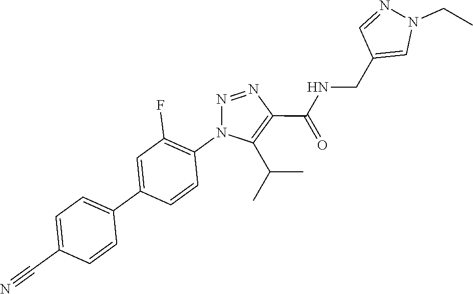 [SMILES: CCn1cc(CNC(=O)c2nnn(-c3ccc(-c4ccc(C#N)cc4)cc3F)c2C(C)C)cn1] | 17-19 | 0.536 | 457.5 | 458.2 | [M + H]⁺ | 4 |
| 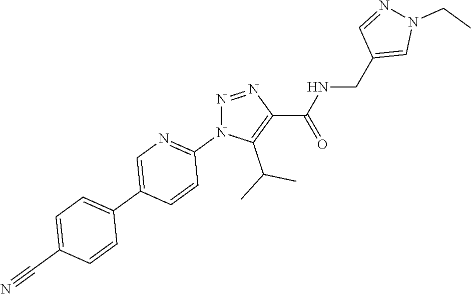 [SMILES: CCn1cc(CNC(=O)c2nnn(-c3ccc(-c4ccc(C#N)cc4)cn3)c2C(C)C)cn1] | 17-20 | 0.505 | 440.5 | 441.2 | [M + H]⁺ | 4 |
| 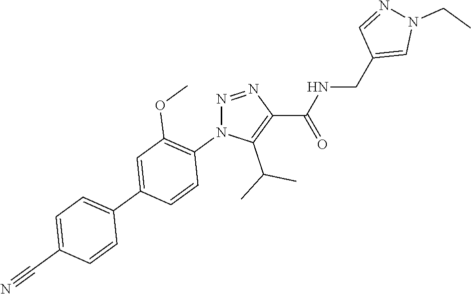 [SMILES: CCn1cc(CNC(=O)c2nnn(-c3ccc(-c4ccc(C#N)cc4)cc3OC)c2C(C)C)cn1] | 17-21 | 0.534 | 469.5 | 470.2 | [M + H]⁺ | 4 |
| 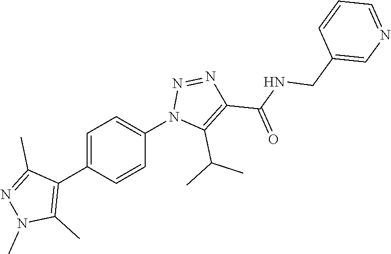 [SMILES: Cc1nn(C)c(C)c1-c1ccc(-n2nnc(C(=O)NCc3cccnc3)c2C(C)C)cc1] | 17-22 | 0.888 | 429.5 | 430.2 | [M + H]⁺ | 5 |
| 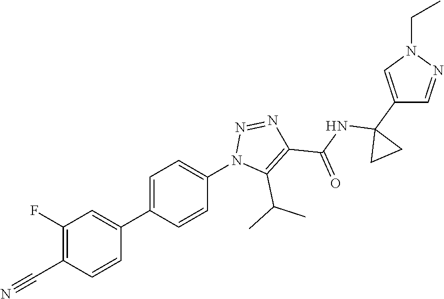 [SMILES: CCn1cc(C2(NC(=O)c3nnn(-c4ccc(-c5ccc(C#N)c(F)c5)cc4)c3C(C)C)CC2)cn1] | 17-23 | 0.887 | 483.6 | 484.2 | [M + H]⁺ | 4 |
| 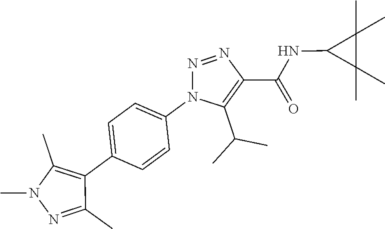 | 17-24 | 1.041 | 434.6 | 435.2 | [M + H]⁺ | 5 |
| 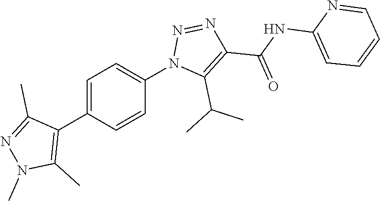 [SMILES: Cc1nn(C)c(C)c1-c1ccc(-n2nnc(C(=O)Nc3ccccn3)c2C(C)C)cc1] | 17-25 | 1.007 | 415.5 | 416.1 | [M + H]⁺ | 5 |
| 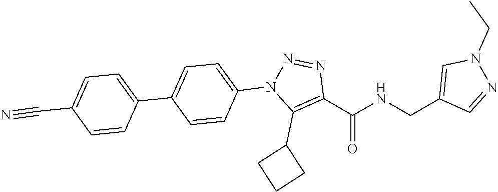 [SMILES: CCn1cc(CNC(=O)c2nnn(-c3ccc(-c4ccc(C#N)cc4)cc3)c2C2CCC2)cn1] | 17-26 | 0.944 | 451.5 | 452.2 | [M + H]⁺ | 5 |
| 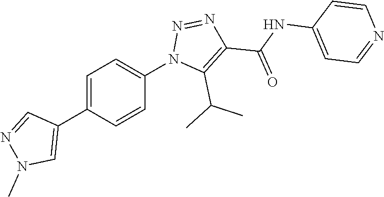 [SMILES: CC(C)c1c(C(=O)Nc2ccncc2)nnn1-c1ccc(-c2cnn(C)c2)cc1] | 17-27 | 5.030, 5.084 | 387.4 | 388 | [M + H]⁺ | 1 |
| 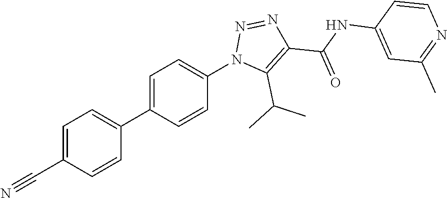 [SMILES: Cc1cc(NC(=O)c2nnn(-c3ccc(-c4ccc(C#N)cc4)cc3)c2C(C)C)ccn1] | 17-28 | 1.011 | 422.5 | 423.1 | [M + H]⁺ | 5 |
| 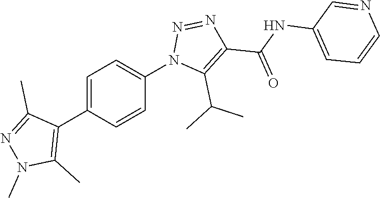 [SMILES: Cc1nn(C)c(C)c1-c1ccc(-n2nnc(C(=O)Nc3cccnc3)c2C(C)C)cc1] | 17-29 | 0.926 | 415.5 | 416.1 | [M + H]⁺ | 5 |
| 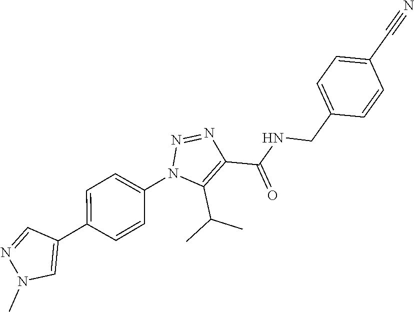 | 17-30 | 9.359 | 411.5 | 412 | [M + H]⁺ | 1 |
| 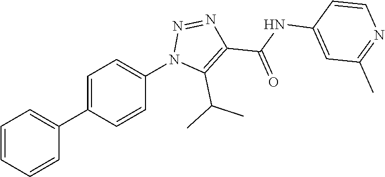 [SMILES: Cc1cc(NC(=O)c2nnn(-c3ccc(-c4ccccc4)cc3)c2C(C)C)ccn1] | 17-31 | 0.987 | 397.5 | 398.2 | [M + H]⁺ | 5 |
| 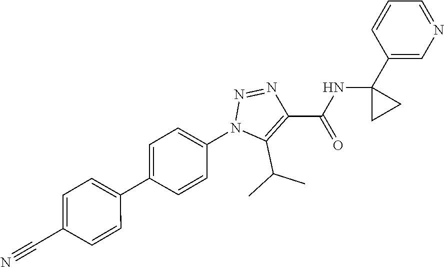 [SMILES: CC(C)c1c(C(=O)NC2(c3cccnc3)CC2)nnn1-c1ccc(-c2ccc(C#N)cc2)cc1] | 17-32 | 0.981 | 448.5 | 449.1 | [M + H]⁺ | 5 |
| 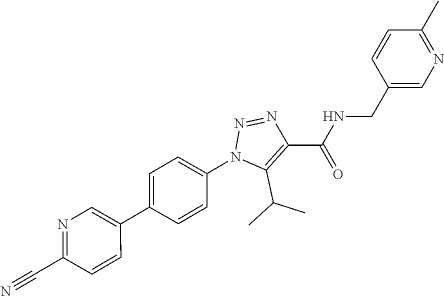 [SMILES: Cc1ccc(CNC(=O)c2nnn(-c3ccc(-c4ccc(C#N)nc4)cc3)c2C(C)C)cn1] | 17-33 | 0.845 | 437.5 | 438.2 | [M + H]⁺ | 5 |
| 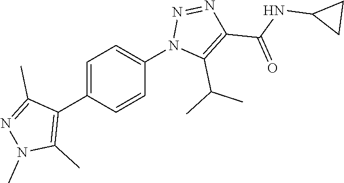 [SMILES: Cc1nn(C)c(C)c1-c1ccc(-n2nnc(C(=O)NC3CC3)c2C(C)C)cc1] | 17-34 | 0.913 | 378.5 | 379.2 | [M + H]⁺ | 5 |

### Example 18

### Synthesis of Compound 18-3 and Other Representative Compounds

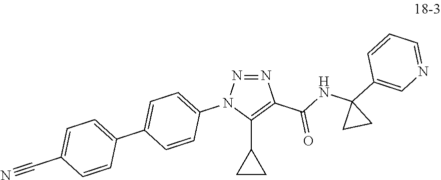 [SMILES: N#Cc1ccc(-c2ccc(-n3nnc(C(=O)NC4(c5cccnc5)CC4)c3C3CC3)cc2)cc1]

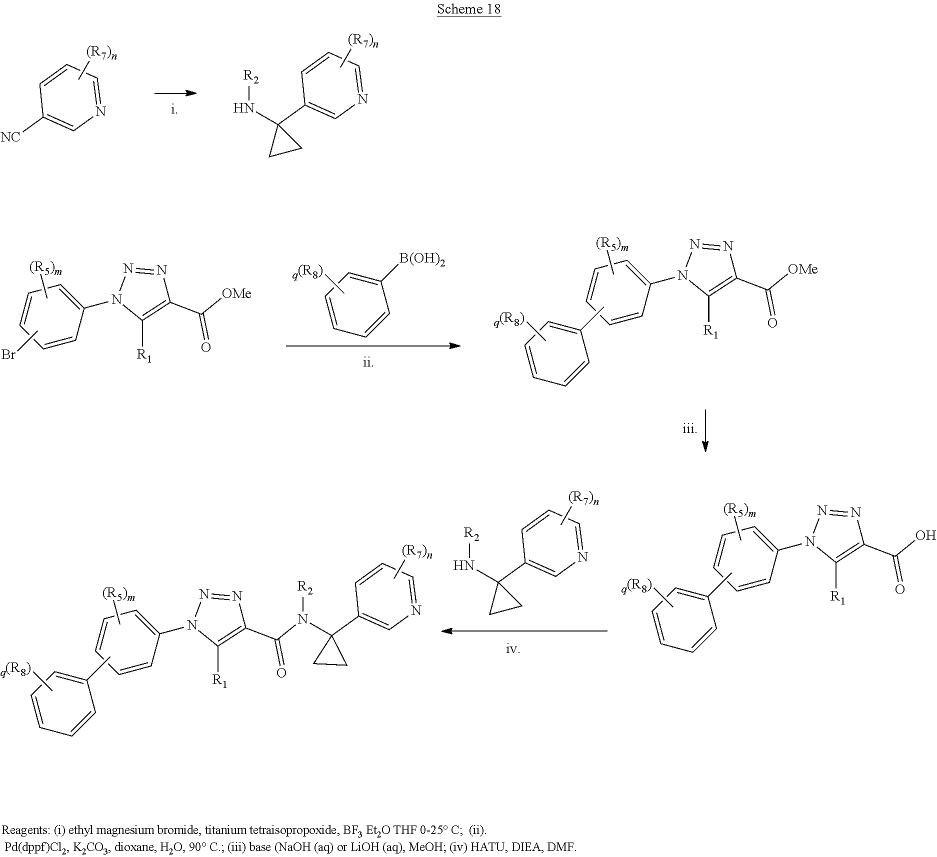

### Step 18-1. Synthesis of 1-(pyridin-3-yl)cyclopropan-1-amine (INT 18A)

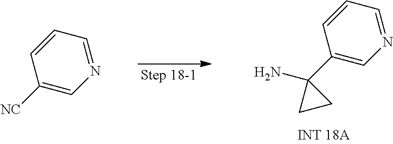 [SMILES: N#Cc1cccnc1 ; NC1(c2cccnc2)CC1]

To a mixture of pyridine-3-carbonitrile (500 mg, 4.80 mmol) in THF (10 mL) were added ethyl magnesium bromide (3 M, 4.00 mL) and titanium tetraisopropoxide (2.13 mL, 7.20 mmol) at −70′° C. The mixture was allowed to slowly warm to 25° C. and stirred for 3 h. After cooling to 0° C., BF₃·Et₂O (1.19 mL, 9.61 mmol,) was added, and the mixture was stirred at 25° C. for 15 h. The mixture was quenched with aqueous 1M NaOH and extracted with EA (3×). The combined organic extracts were dried (Na₂SO₄), filtered and concentrated under reduced pressure to give a residue that was purified by SiO₂ chromatography (MeOH/EA) to afford 200 mg (18.6%) of 1-(pyridin-3-yl) cyclopropan-1-amine (INT 18A). LCMS-ESI (m/z) calculated for C₈H₁₀N₂: 134.1; found 135.2 [M+H]⁺, t_{R}=0.358 min (Method 5). ¹H NMR (400 MHz, CDCl3) δ 8.52 (d, J=1.9 Hz, 1H), 8.37 (dd, J=1.6, 4.8 Hz, 1H), 7.56-7.49 (in, 1H), 7.16 (ddd, J=0.8, 4.8, 8.0 Hz, 1H), 2.28 (br s, 2H), 1.09-1.04 (m, 2H), 0.98-0.92 (m, 2H).

### Step 18-2. Synthesis of methyl 1-(4′-cyano-[1,1′-biphenyl]-4-yl)-5-cyclopropyl-1H-1,2,3-triazole-4-carboxylate (18 B)

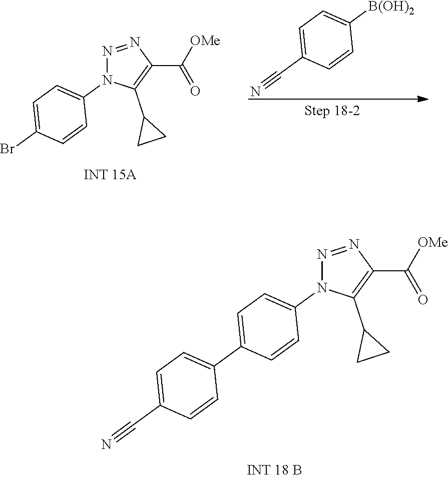 [SMILES: COC(=O)c1nnn(-c2ccc(Br)cc2)c1C1CC1 ; N#Cc1ccc(B(O)O)cc1 ; COC(=O)c1nnn(-c2ccc(-c3ccc(C#N)cc3)cc2)c1C1CC1]

To a mixture of (4-cyanophenyl) boronic acid (900 mg, 3.33 mmol, 1.52 eq) in dioxane (15 mL) and H₂O (3 mL), were added INT 15A (1.3 g, 4.04 mmol), Pd(dppf)Cl₂ (295.3 mg, 403 μmol, 0.1 eq) and K₂CO₃ (1.12 g, 8.1 mmol). The solution was de-gassed and then heated to 90° C. for 15 hours under N₂. The reaction mixture was concentrated to a residue that was purified by SiO₂ chromatography (EA/petroleum ether) to afford 1.3 g (90.7%) of methyl 1-(4′-cyano-[1,1′-biphenyl]-4-yl)-5-cyclopropyl-1H-1,2,3-triazole-4-carboxylate (INT 18B) as a yellow solid. LCMS-ESI (m/z) calculated for C₂₀H₁₆N₄O₂: 344.4; found 345.1 [M+H]⁺, t_{R}=0.846 min(Method 5).

### Step 18-3. Synthesis of 1-(4′-cyano-[1,1′-biphenyl]-4-yl)-5-cyclopropyl-1H-1,2,3-triazole-4-carboxylic acid (NT 18C)

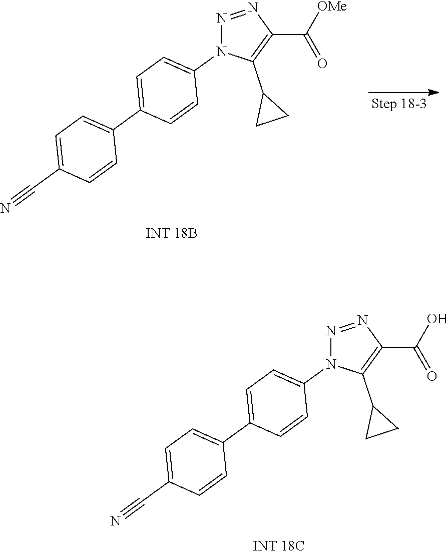 [SMILES: COC(=O)c1nnn(-c2ccc(-c3ccc(C#N)cc3)cc2)c1C1CC1 ; N#Cc1ccc(-c2ccc(-n3nnc(C(=O)O)c3C3CC3)cc2)cc1]

To a solution of INT 18B (1.3 g, 3.78 mmol) in MeOH (20 mL) was added LiOH (1 M, 7.55 mL, 7.55 mmol). The reaction mixture was stirred at 25° C. for 20 h then concentrated to remove MeOH. The resulting aqueous residue was pH adjusted to pH=7 with 1M HCl, creating a yellow precipitate that was collected by filtration to afford 1.2 g of crude 1-(4′-cyano-[1,1′-biphenyl]-4-yl)-5-cyclopropyl-1H-1,2,3-triazole-4-carboxylic acid (INT 18C) as a yellow solid (47% purity) that was used without further purification. LCMS-ESI (m/z) calculated for C₁₉H₁₄N₄O₂: 330.3; found 331.2 [M+H]⁺, t_{R}=0.97 min(Method 4).

### Step 18-4. Synthesis of 1-(4′-cyano-[1,1′-biphenyl]-4-yl)-5-cyclopropyl-N-(1-(pyridin-3-yl)cyclopropyl)-1H-1,2,3-triazole-4-carboxamide (Compound 18-3)

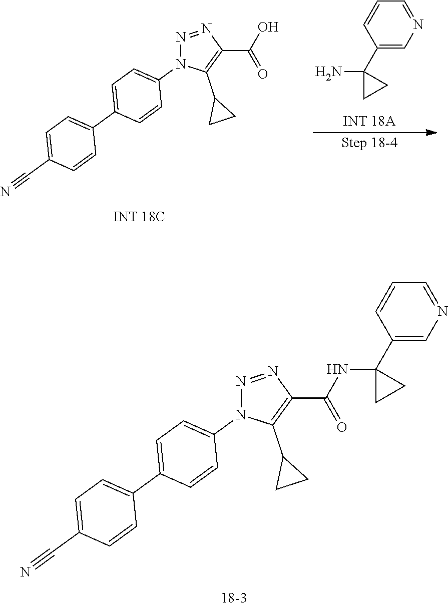 [SMILES: N#Cc1ccc(-c2ccc(-n3nnc(C(=O)O)c3C3CC3)cc2)cc1 ; NC1(c2cccnc2)CC1 ; N#Cc1ccc(-c2ccc(-n3nnc(C(=O)NC4(c5cccnc5)CC4)c3C3CC3)cc2)cc1]

To a solution of INT 18C (100 mg of 47% purity, 143 μmol) and INT 18A (103.3 mg, 605.4 μmol) in DMF (3 mL) were added HATU (230 mg, 605.4 μmol) and DIEA (210 μL, 1.21 μmol). After stirring at 25° C. for 1 h, the mixture was diluted with saturated, aqueous NaHCO₃ and extracted with EA. The combined organic layers were dried (Na₂SO₄), filtered, and concentrated to give a residue that was purified by prep-HPLC (aqueous NH₄HCO₃/CH₃CN) and then prep HPLC (H₂O (FA)/CH₃CN) to provide 37.6 mg (27.5%) of 1-(4′-cyano-[1,1′-biphenyl]-4-yl)-5-cyclopropyl-N-(1-(pyridin-3-yl)cyclopropyl)-1H-1,2,3-triazole-4-carboxamide (Compound 18-3) as a yellow solid. LCMS-ESI (m/z) calculated for C₂₇H₂₂N₆O: 446.5; found 447.2 [M+H]⁺, t_{R}=0.817 min(Method 4). ¹H NMR (400 MHz, CDCl₃) δ=8.65 (br s, 1H), 8.58-8.42 (m, 1H), 8.15-8.01 (m, 1H), 7.84-7.74 (m, 7H), 7.68 (br d, J=8.3 Hz, 2H), 7.44 (br s, 1H), 2.03-1.93 (m, 1H), 1.55 (br s, 2H), 1.46 (br s, 2H), 1.10 (br d, J=4.0 Hz, 2H), 1.03 (brd, J=8.4 Hz, 2H).

The compounds listed in Table 18 were made using the procedures of Scheme 18.

TABLE 18

| | | Purity | | | | |
|---|---|---|---|---|---|---|
| | Cpd | RT | | observed | | Purity |
| Structure | No. | (min) | MW | m/z | Ion | Method |
| 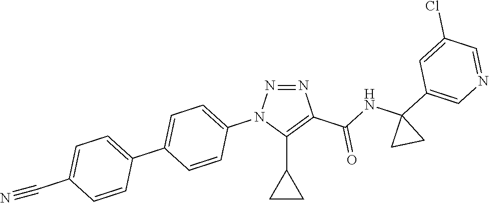 [SMILES: N#Cc1ccc(-c2ccc(-n3nnc(C(=O)NC4(c5cncc(Cl)c5)CC4)c3C3CC3)cc2)cc1] | 18-1 | 0.933 | 481.0 | 481.2 | [M + H]⁺ | 4 |
| 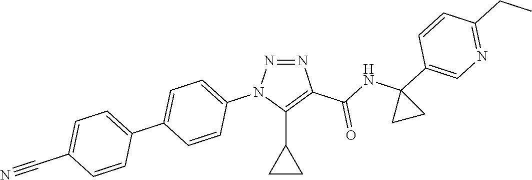 [SMILES: CCc1ccc(C2(NC(=O)c3nnn(-c4ccc(-c5ccc(C#N)cc5)cc4)c3C3CC3)CC2)cn1] | 18-2 | 0.816 | 474.6 | 475.3 | [M + H]⁺ | 4 |
| 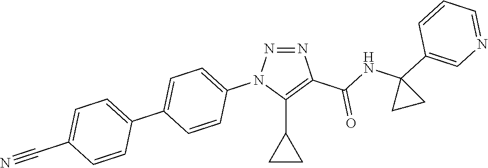 [SMILES: N#Cc1ccc(-c2ccc(-n3nnc(C(=O)NC4(c5cccnc5)CC4)c3C3CC3)cc2)cc1] | 18-3 | 0.819 | 446.5 | 447.2 | [M + H]⁺ | 4 |
| 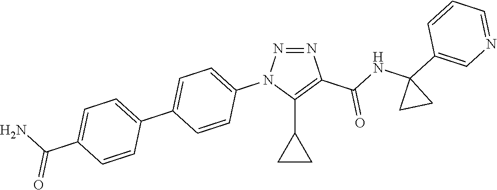 [SMILES: NC(=O)c1ccc(-c2ccc(-n3nnc(C(=O)NC4(c5cccnc5)CC4)c3C3CC3)cc2)cc1] | 18-4 | 0.743 | 464.5 | 465.3 | [M + H]⁺ | 4 |
| 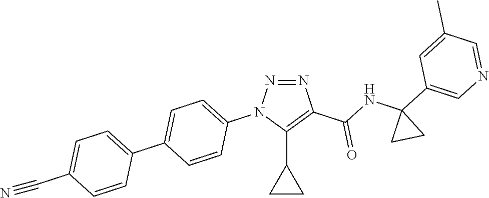 [SMILES: Cc1cncc(C2(NC(=O)c3nnn(-c4ccc(-c5ccc(C#N)cc5)cc4)c3C3CC3)CC2)c1] | 18-5 | 0.817 | 460.5 | 461 | [M + H]⁺ | 4 |
| 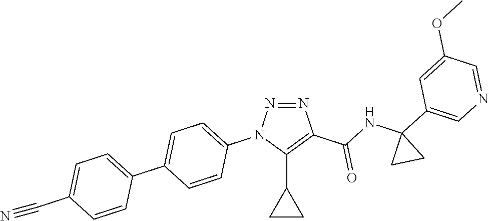 [SMILES: COc1cncc(C2(NC(=O)c3nnn(-c4ccc(-c5ccc(C#N)cc5)cc4)c3C3CC3)CC2)c1] | 18-6 | 0.832 | 476.5 | 477 | [M + H]⁺ | 4 |
| 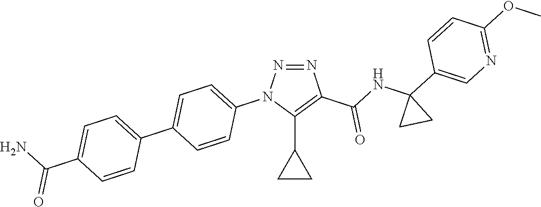 [SMILES: COc1ccc(C2(NC(=O)c3nnn(-c4ccc(-c5ccc(C(N)=O)cc5)cc4)c3C3CC3)CC2)cn1] | 18-7 | 0.843 | 494.6 | 495.4 | [M + H]⁺ | 4 |
| 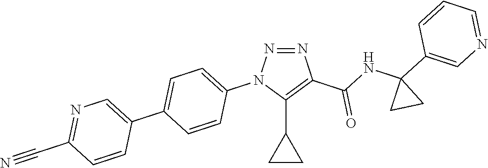 [SMILES: N#Cc1ccc(-c2ccc(-n3nnc(C(=O)NC4(c5cccnc5)CC4)c3C3CC3)cc2)cn1] | 18-8 | 0.892 | 447.5 | 448.1 | [M + H]⁺ | 5 |
| 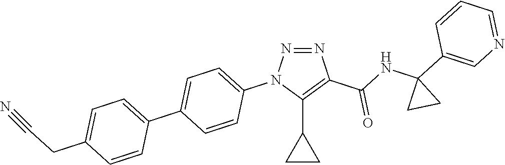 [SMILES: N#CCc1ccc(-c2ccc(-n3nnc(C(=O)NC4(c5cccnc5)CC4)c3C3CC3)cc2)cc1] | 18-9 | 0.804 | 460.5 | 461.3 | [M + H]⁺ | 4 |
| 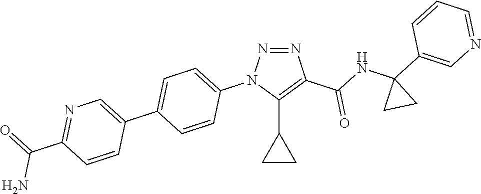 [SMILES: NC(=O)c1ccc(-c2ccc(-n3nnc(C(=O)NC4(c5cccnc5)CC4)c3C3CC3)cc2)cn1] | 18-10 | 0.754 | 465.5 | 466.2 | [M + H]⁺ | 5 |
| 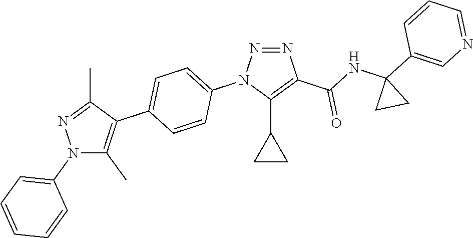 [SMILES: Cc1nn(-c2ccccc2)c(C)c1-c1ccc(-n2nnc(C(=O)NC3(c4cccnc4)CC3)c2C2CC2)cc1] | 18-11 | 0.890 | 515.6 | 516.2 | [M + H]⁺ | 5 |
| 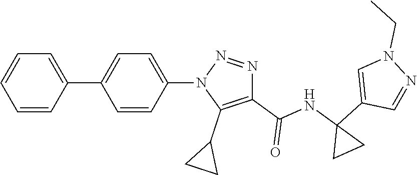 [SMILES: CCn1cc(C2(NC(=O)c3nnn(-c4ccc(-c5ccccc5)cc4)c3C3CC3)CC2)cn1] | 18-12 | 0.974 | 438.5 | 439.2 | [M + H]⁺ | 5 |
| 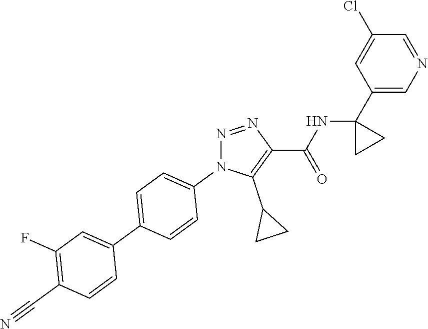 [SMILES: N#Cc1ccc(-c2ccc(-n3nnc(C(=O)NC4(c5cncc(Cl)c5)CC4)c3C3CC3)cc2)cc1F] | 18-13 | 0.697 | 498.9 | 499 | [M + H]⁺ | 4 |
| 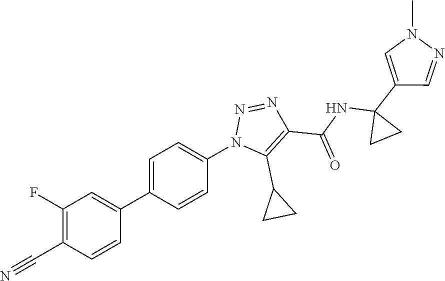 | 18-14 | 0.913 | 481.5 | 482 | [M + H]⁺ | 4 |
| 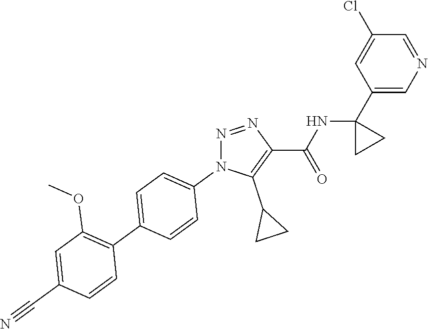 [SMILES: COc1cc(C#N)ccc1-c1ccc(-n2nnc(C(=O)NC3(c4cncc(Cl)c4)CC3)c2C2CC2)cc1] | 18-15 | 0.961 | 511.0 | 510.9 | [M + H]⁺ | 4 |
| 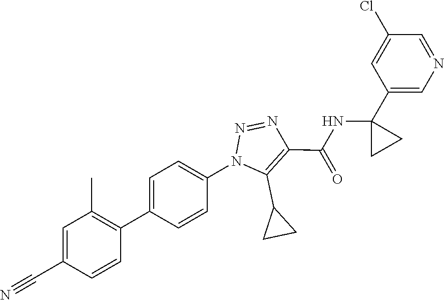 [SMILES: Cc1cc(C#N)ccc1-c1ccc(-n2nnc(C(=O)NC3(c4cncc(Cl)c4)CC3)c2C2CC2)cc1] | 18-16 | 0.971 | 495.0 | 495 | [M + H]⁺ | 4 |
| 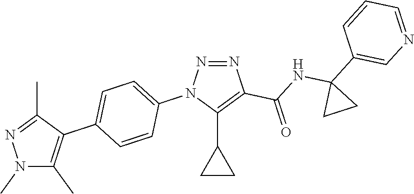 [SMILES: Cc1nn(C)c(C)c1-c1ccc(-n2nnc(C(=O)NC3(c4cccnc4)CC3)c2C2CC2)cc1] | 18-17 | 10.06 | 453.6 | 454 | [M + H]⁺ | 2 |
| 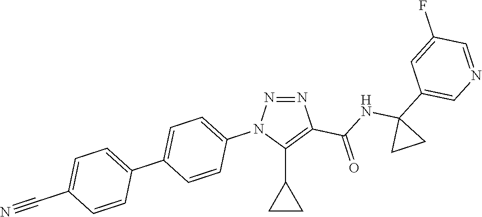 [SMILES: N#Cc1ccc(-c2ccc(-n3nnc(C(=O)NC4(c5cncc(F)c5)CC4)c3C3CC3)cc2)cc1] | 18-18 | 0.923 | 464.5 | 465 | [M + H]⁺ | 4 |
| 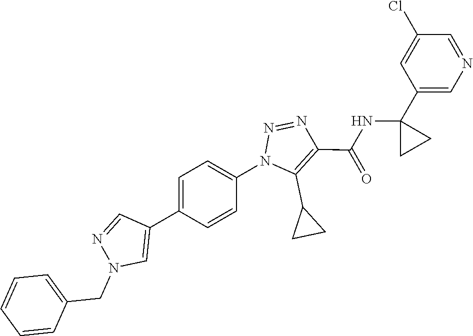 [SMILES: O=C(NC1(c2cncc(Cl)c2)CC1)c1nnn(-c2ccc(-c3cnn(Cc4ccccc4)c3)cc2)c1C1CC1] | 18-19 | 0.705 | 536.0 | 536.1 | [M + H]⁺ | 4 |
| 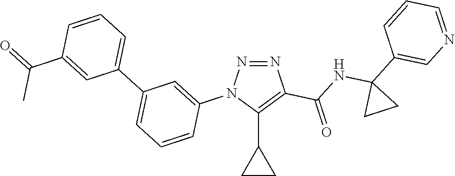 [SMILES: CC(=O)c1cccc(-c2cccc(-n3nnc(C(=O)NC4(c5cccnc5)CC4)c3C3CC3)c2)c1] | 18-20 | 0.836 | 463.5 | 464.2 | [M + H]⁺ | 5 |
| 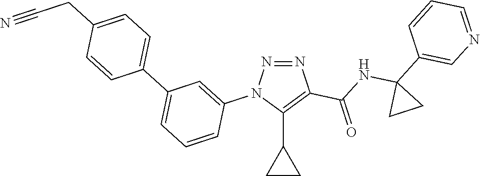 [SMILES: N#CCc1ccc(-c2cccc(-n3nnc(C(=O)NC4(c5cccnc5)CC4)c3C3CC3)c2)cc1] | 18-21 | 0.980 | 460.5 | 461.3 | [M + H]⁺ | 5 |
| 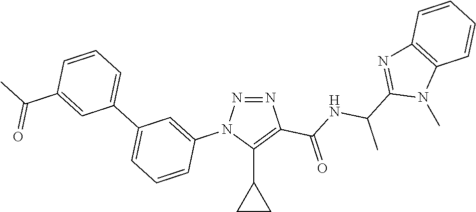 [SMILES: CC(=O)c1cccc(-c2cccc(-n3nnc(C(=O)NC(C)c4nc5ccccc5n4C)c3C3CC3)c2)c1] | 18-22 | 0.814 | 504.6 | 505.2 | [M + H]⁺ | 4 |
| 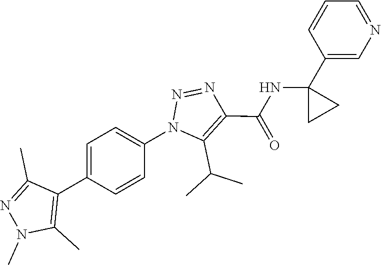 [SMILES: Cc1nn(C)c(C)c1-c1ccc(-n2nnc(C(=O)NC3(c4cccnc4)CC3)c2C(C)C)cc1] | 18-23 | 0.908 | 455.6 | 456.1 | [M + H]⁺ | 5 |
| 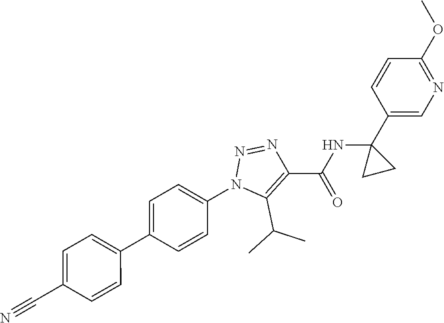 [SMILES: COc1ccc(C2(NC(=O)c3nnn(-c4ccc(-c5ccc(C#N)cc5)cc4)c3C(C)C)CC2)cn1] | 18-24 | 1.003 | 478.6 | 479.2 | [M + H]⁺ | 5 |
| 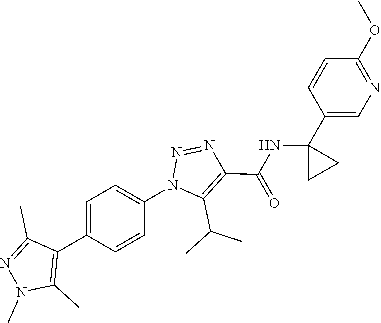 [SMILES: COc1ccc(C2(NC(=O)c3nnn(-c4ccc(-c5c(C)nn(C)c5C)cc4)c3C(C)C)CC2)cn1] | 18-25 | 0.891 | 485.6 | 486.1 | [M + H]⁺ | 4 |
| 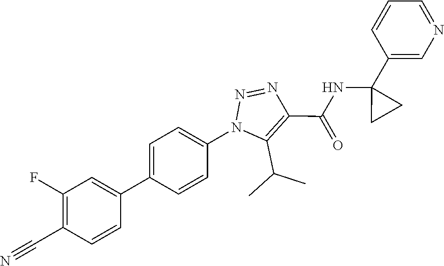 [SMILES: CC(C)c1c(C(=O)NC2(c3cccnc3)CC2)nnn1-c1ccc(-c2ccc(C#N)c(F)c2)cc1] | 18-26 | 0.945 | 466.5 | 467.1 | [M + H]⁺ | 5 |
| 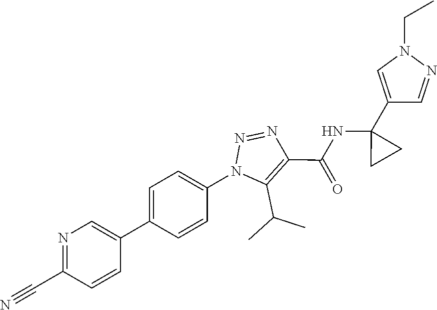 [SMILES: CCn1cc(C2(NC(=O)c3nnn(-c4ccc(-c5ccc(C#N)nc5)cc4)c3C(C)C)CC2)cn1] | 18-27 | 0.835 | 466.5 | 467.2 | [M + H]⁺ | 4 |
| 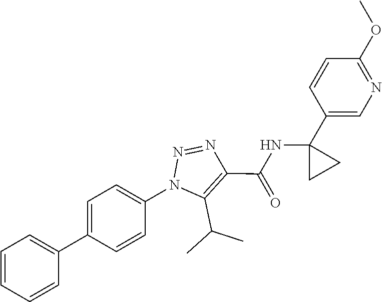 [SMILES: COc1ccc(C2(NC(=O)c3nnn(-c4ccc(-c5ccccc5)cc4)c3C(C)C)CC2)cn1] | 18-28 | 1.036 | 453.5 | 454.2 | [M + H]⁺ | 5 |
| 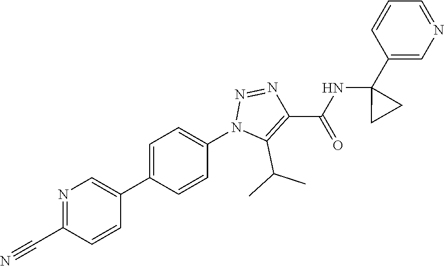 [SMILES: CC(C)c1c(C(=O)NC2(c3cccnc3)CC2)nnn1-c1ccc(-c2ccc(C#N)nc2)cc1] | 18-29 | 0.915 | 449.5 | 450.1 | [M + H]⁺ | 5 |
| 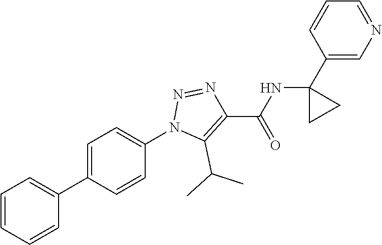 [SMILES: CC(C)c1c(C(=O)NC2(c3cccnc3)CC2)nnn1-c1ccc(-c2ccccc2)cc1] | 18-30 | 1.025 | 423.5 | 424.1 | [M + H]⁺ | 5 |

### Example 19

### Synthesis of Compound 19-2 and Other Representative Compounds

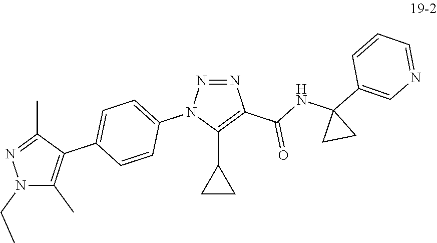 [SMILES: CCn1nc(C)c(-c2ccc(-n3nnc(C(=O)NC4(c5cccnc5)CC4)c3C3CC3)cc2)c1C]

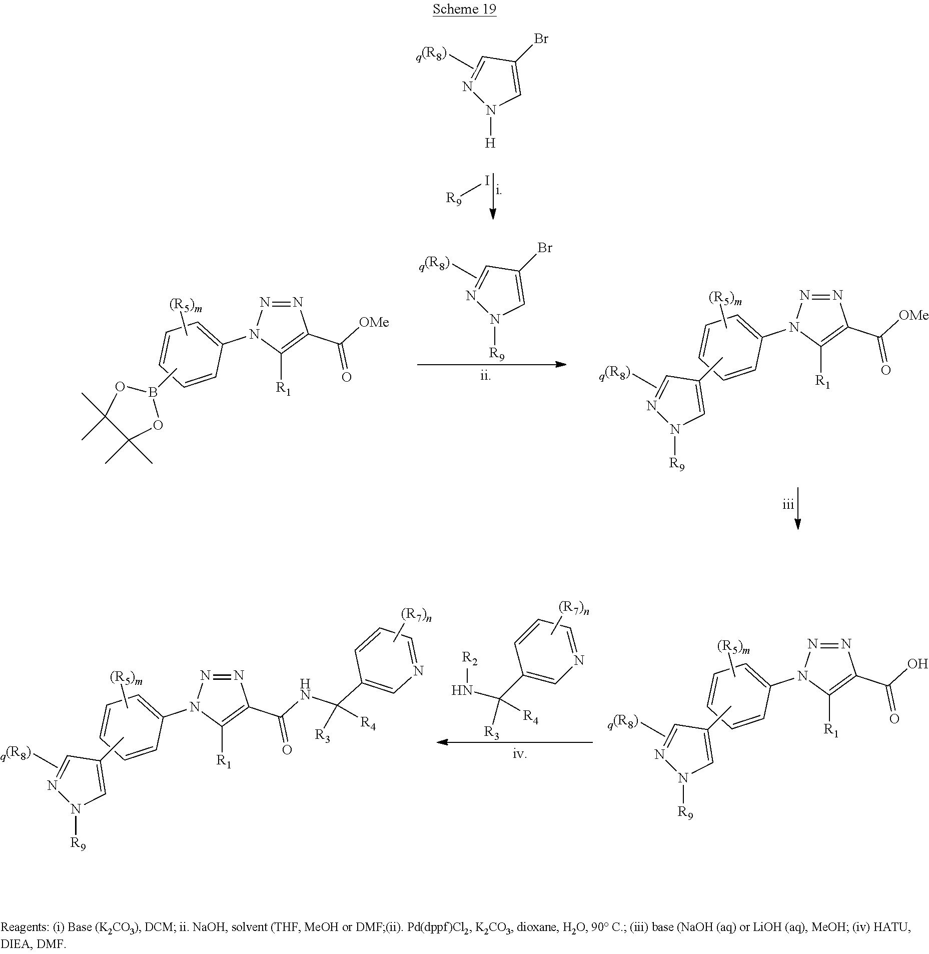

### Step 19-1. Synthesis of 4-bromo-1-ethyl-3,5-dimethyl-1H-pyrazole (INT 19A)

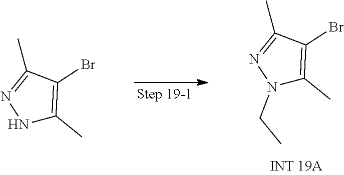

To a solution of 4-bromo-3,5-dimethyl-1H-pyrazole (2 g, 11.43 mmol) in CH₃CN (20 mL) were added K₂CO₃ (4.74 g, 34.28 mmol) and iodoethane (1.83 mL, 22.85 mmol). The mixture was stirred at 80° C. for 15 h and then filtered. The filtrate was concentrated and purified by SiO₂ chromatography (EA/petroleum ether) to afford 1.8 g (77.6%) of 4-bromo-1l-ethyl-3,5-dimethyl-1H-pyrazole (INT 19A). LCMS-ESI (m/z) calculated for C₇H₁₁BrN₂: 203.1, found 205.1 [M+H]⁺, t_{R}˜=0.908 min (Method 5). ¹H NMR (400 MHz, CDCl₃) δ 4.02 (q, J=7.3 Hz, 2H), 2.20 (d, J=9.8 Hz, 6H), 1.35 (t, J=7.2 Hz, 3H).

### Step 19-2. Synthesis of methyl 5-cyclopropyl-1-(4-(1-ethyl-3,5-dimethyl-1H-pyrazol-4-yl)phenyl)-1H-1,2,3-triazole-4-carboxylate (INT 19B)

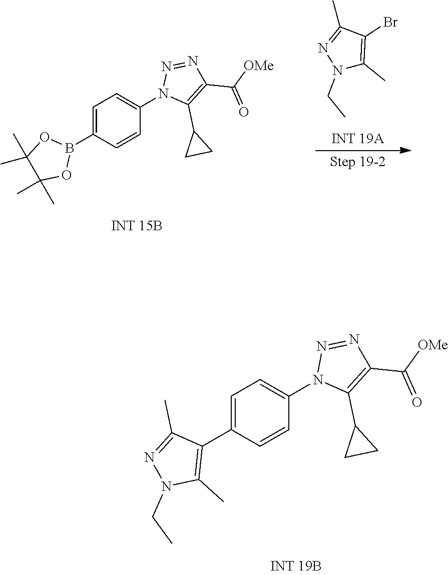

To a solution of INT 15B (2 g, 2.98 mmol) in dioxane (20 mL) and H₂O (5 mL) were added INT 19A (lg, 4.92 mmol), Pd(dppf)Cl₂ (218 mg, 0.298 mmol) and K₂CO₃ (823 mg, 5.92 mmol). The reaction mixture was degassed and stirred at 90° C. for 15 h under an atmosphere of N₂. The reaction mixture was concentrated to a residue that was purified by SiO₂ chromatography (EA/pet ether) to produce 900 mg (83%) of methyl 5-cyclopropyl-1-(4-(1-ethyl-3,5-dimethyl-1H-pyrazol-4-yl)phenyl)-1H-1,2,3-triazole-4-carboxylate (INT 19B) as a yellow solid. LCMS-ESI (m/z) calculated for C₂₀H₂₃N₅O₂: 365.4; found 366.1 [M+H]⁺, t_{R}=0.867 min (Method 5). ¹H NMR (400 MHz, DMSO-D₆) δ=7.57 (d, J=8.4 Hz, 2H), 7.43 (d, J=8.4 Hz, 2H), 4.12 (q, J=7.2 Hz, 2H), 3.99 (s, 3H), 2.30 (s, 6H), 2.07-1.99 (m, 1H), 1.46 (t, J=7.3 Hz, 3H), 1.09-1.02 (m, 2H), 1.00-0.93 (m, 2H).

### Step 19-3. Synthesis of 5-cyclopropyl-1-(4-(1-ethyl-3,5-dimethyl-1H-pyrazol-4-yl)phenyl)-1H-1,2,3-triazole-4-carboxylic acid (INT 19C)

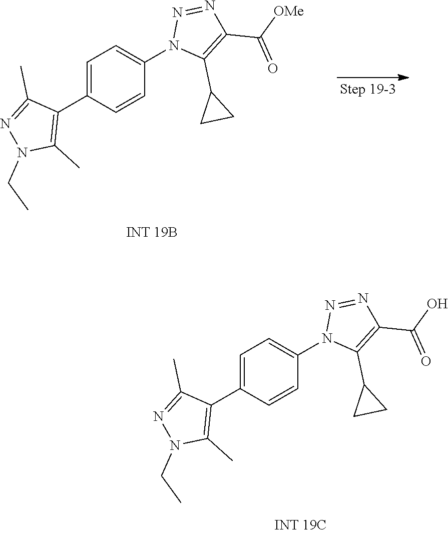 [SMILES: CCn1nc(C)c(-c2ccc(-n3nnc(C(=O)OC)c3C3CC3)cc2)c1C ; CCn1nc(C)c(-c2ccc(-n3nnc(C(=O)O)c3C3CC3)cc2)c1C]

To a solution of INT 19B (900 mg, 2.46 mmol) in MeOH (30 mL) was added LiOH (1 M, 6 mL, 6 mmol). The mixture was stirred at 25° C. for 15 h and then concentrated to remove MeOH. The resulting aqueous solution was pH adjusted to pH=5 using 1M HCl, creating a yellow precipitate that was collected by filtration and dried under vacuum to provide 600 mg (68%) of 5-cyclopropyl-1-(4-(1-ethyl-3,5-dimethyl-1H-pyrazol-4-yl)phenyl)-1H-1,2,3-triazole-4-carboxylic acid (INT 19C) as a yellow solid. LCMS-ESI (m/z) calculated for C₁₉H₂₁N₅O₂: 351.4; found 352.0 [M+H]⁺, t_{R}=0.815 min (Method 4). ¹H NMR (400 MHz, DMSO-D₆) δ=7.68 (d, J=8.4 Hz, 2H), 7.50 (d, J=8.6 Hz, 2H), 4.17-3.98 (m, 2H), 2.29 (s, 3H), 2.20 (s, 3H), 2.16-2.05 (m, 1H), 1.33 (t, J=7.2 Hz, 3H), 0.95-0.87 (m, 2H), 0.77-0.68 (m, 2H).

### Step 19-4. Synthesis of 5-cyclopropyl-1-(4-(1-ethyl-3,5-dimethyl-1H-pyrazol-4-yl)phenyl)-N-(1-(pyridin-3-yl)cyclopropyl)-1H-1,2,3-triazole-4-carboxamideide (Compound 19-2)

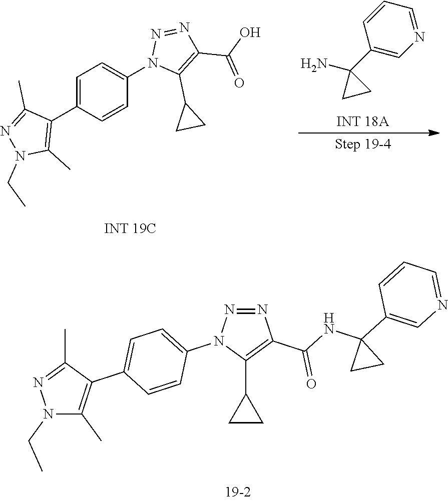 [SMILES: CCn1nc(C)c(-c2ccc(-n3nnc(C(=O)O)c3C3CC3)cc2)c1C ; NC1(c2cccnc2)CC1 ; CCn1nc(C)c(-c2ccc(-n3nnc(C(=O)NC4(c5cccnc5)CC4)c3C3CC3)cc2)c1C]

To a stirring mixture of INT 19C (80 mg, 227.7 μmol) and INT 18A (61 mg, 455.3 μmol) in DMF (3 mL) were added HATU (173.1 mg, 455.3 μmol) and DIEA (119 μL, 683 μmol). After stirring at 25° C. for 1 h, the mixture was diluted with saturated, aqueous NaHCO₃ and extracted with EA (2×). The combined organic layers were washed with brine, dried (Na₂SO₄), concentrated, and purified by prep HPLC (10 mM aqueous NH₄CO₃/CH₃CN) to produce 53.29 mg (50%) of 5-cyclopropyl-1-(4-(1-ethyl-3,5-dimethyl-1H-pyrazol-4-yl)phenyl)-N-(1-(pyridin-3-yl)cyclopropyl)-1H-1,2,3-triazole-4-carboxamide (Compound 19-2) as a white solid. LCMS-ESI (m/z) calculated for C₂₇H₂₉N₇O: 467.6; found 468.0 [M+H]⁺, t_{R}=0.778 min (Method 4). ¹H NMR (400 MHz, CDCl₃) δ=8.61 (d, J=1.7 Hz, 1H), 8.47 (dd, J=1.6, 4.9 Hz, 1H), 8.04 (s, 1H), 7.70 (td, J=2.0, 8.0 Hz, 1H), 7.58-7.53 (m, 2H), 7.47-7.41 (m, 2H), 7.30-7.25 (m, 1H), 4.13 (q, J=7.2 Hz, 2H), 2.31 (s, 6H), 1.98 (tt, J=5.4, 8.6 Hz, 1H), 1.50-1.40 (m, 7H), 1.20-1.13 (m, 2H), 1.05-0.96 (m, 2H).

The compounds listed in Table 19 were made using the procedures of Scheme 19.

TABLE 19

| | | Purity | | | | |
|---|---|---|---|---|---|---|
| | Cpd | RT | | observed | | Purity |
| Structure | No. | (min) | MW | m/z | Ion | Method |
| 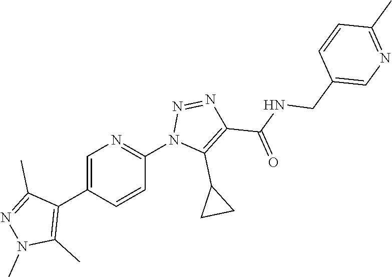 [SMILES: Cc1ccc(CNC(=O)c2nnn(-c3ccc(-c4c(C)nn(C)c4C)cn3)c2C2CC2)cn1] | 19-1 | 0.35 | 442.5 | 443.3 | [M + H]⁺ | 4 |
| 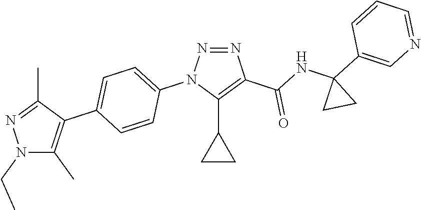 [SMILES: CCn1nc(C)c(-c2ccc(-n3nnc(C(=O)NC4(c5cccnc5)CC4)c3C3CC3)cc2)c1C] | 19-2 | 0.774 | 467.6 | 468 | [M + H]⁺ | 4 |
| 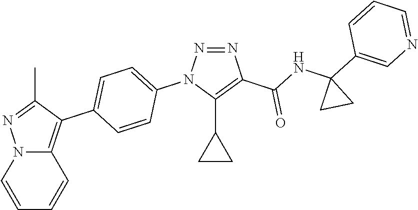 [SMILES: Cc1nn2ccccc2c1-c1ccc(-n2nnc(C(=O)NC3(c4cccnc4)CC3)c2C2CC2)cc1] | 19-3 | 0.801 | 475.6 | 476 | [M + H]⁺ | 4 |
| 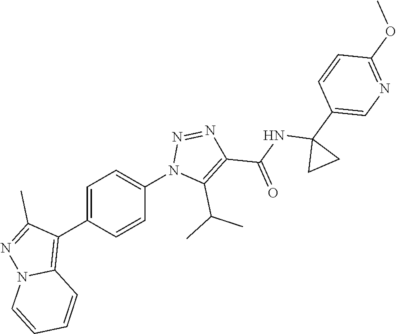 [SMILES: COc1ccc(C2(NC(=O)c3nnn(-c4ccc(-c5c(C)nn6ccccc56)cc4)c3C(C)C)CC2)cn1] | 19-4 | 0.965 | 507.6 | 508 | [M + H]⁺ | 4 |

### Example 20

### Synthesis of Compound 20-1 and Other Representative Compounds

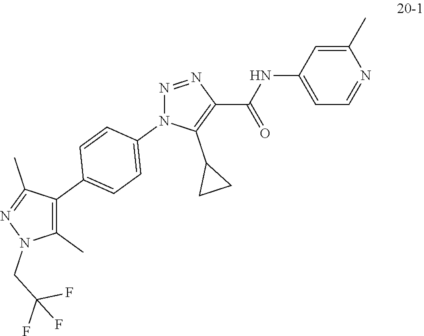 [SMILES: Cc1cc(NC(=O)c2nnn(-c3ccc(-c4c(C)nn(CC(F)(F)F)c4C)cc3)c2C2CC2)ccn1]

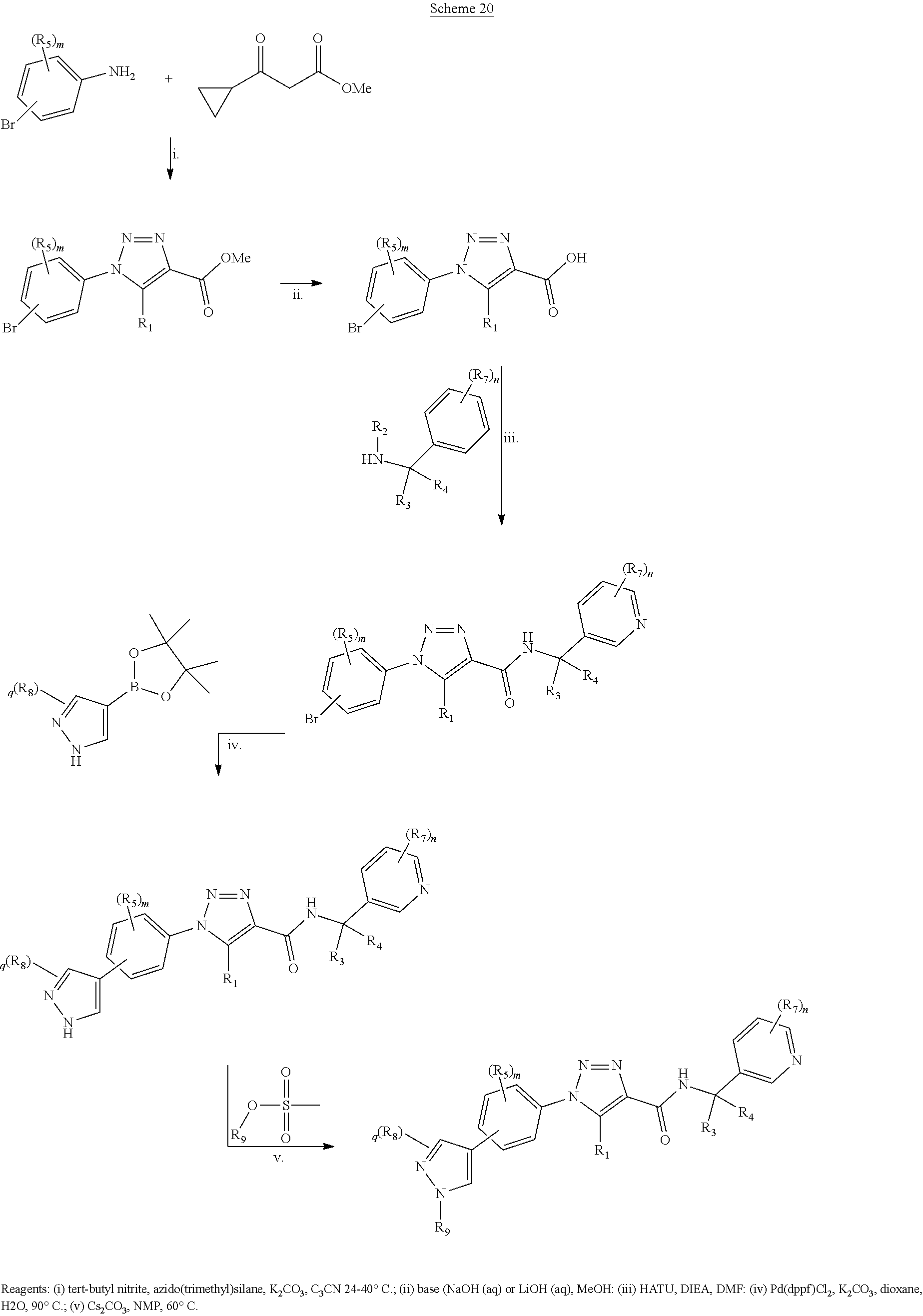

### Step 20-1. Synthesis of ethyl 1-(4-bromophenyl)-5-cyclopropyl-1H-1,2,3-triazole-4-carboxylate (INT 20A)

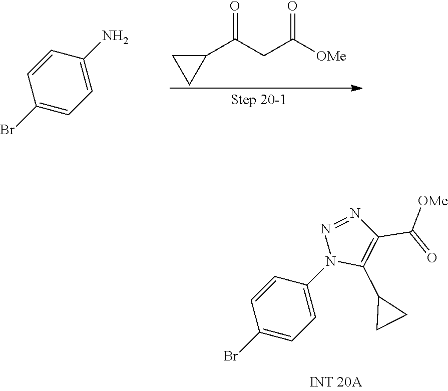 [SMILES: Nc1ccc(Br)cc1 ; COC(=O)CC(=O)C1CC1 ; COC(=O)c1nnn(-c2ccc(Br)cc2)c1C1CC1]

To a solution of 4-bromoaniline (5 g, 29.07 mmol) in CH₃CN (100 mL) at 0° C. were added tert-butyl nitrite (5.19 mL, 43.6 mmol) and azido (trimethyl)silane (4.59 mL, 34.9 mmol). After stirring at 25° C. for 2 h, methyl 3-cyclopropyl-3-oxo-propanoate (4.96 g, 34.89 mmol) and K₂CO₃ (12.05 g, 87.20 mmol) were added, and the reaction mixture was stirred at 40° C. for 12 h. The reaction mixture was poured into saturated, aqueous NaHCO₃ solution at 0° C., and extracted with EA (3×). The combined organic layers were washed with brine (2×), dried (Na₂SO₄), filtered and concentrated under vacuum. The resulting residue was purified by SiO₂ chromatography (EA/petroleum ether) to afford 5.2 g (55.5%) of methyl 1-(4-bromophenyl)-5-cyclopropyl-1H-1,2,3-triazole-4-carboxylate (INT 20A) as a yellow solid. LCMS-ESI (m/z) calculated for C₁₃H₁₂BrN₃O₂: 322.2; found 323.7 [M+H]⁺, t_{R}=0.244 min (Method 7). ¹H NMR (400 MHz, DMSO-D₆) δ=7.85 (d, J=8.6 Hz, 2H), 7.67 (d, J=8.6 Hz, 2H), 3.87 (s, 3H), 2.09 (ddd, J=3.1, 5.5, 8.6 Hz, 1H), 0.99-0.83 (m, 2H), 0.68-0.52 (m, 2H).

### Step 20-2. Synthesis of 1-(4-bromophenyl)-5-cyclopropyl-1H-1,2,3-triazole-4-carboxylic acid (INT 20B)

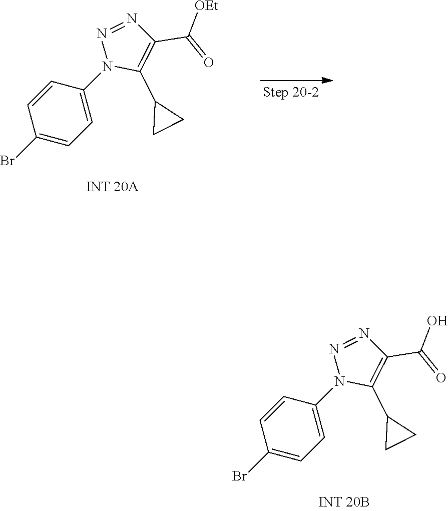 [SMILES: CCOC(=O)c1nnn(-c2ccc(Br)cc2)c1C1CC1 ; O=C(O)c1nnn(-c2ccc(Br)cc2)c1C1CC1]

To a solution of INT 20A (1.0 g, 3.1 mmol) in MeOH (6 mL) and H₂O (2 mL) was added LiOH·H₂O (390.8 mg, 9.3 mmol). After the reaction was stirred at 25° C. for 2 h, the reaction mixture was adjusted using 1 M HCl solution to pH 6-7 and concentrated in vacuo to give 950 mg (99.3%) of 1-(4-bromophenyl)-5-cyclopropyl-1H-1,2,3-triazole-4-carboxylic acid (INT 20B) of yellow solid that was used without further purification in the next step. LCMS-ESI (m/z) calculated for C₁₂H₁₂BrN₃O₂: 308.1; found 310.0 [M+H]⁺, t_{R}=0.437 min (Method 7).

### Step 20-3. Synthesis of 1-(4-bromophenyl)-5-cyclopropyl-N-(2-methylpyridin-4-yl)-1H-1,2,3-triazole-4-carboxamide (INT 20C)

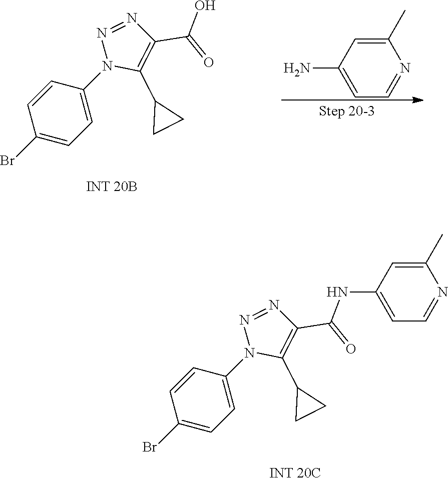

To a solution of INT 20B (1.13 g, 3.67 mmol) and 2-methylpyridin-4-amine (436.24 mg, 4.03 mmol) in DCE (10 mL) were added 2-chloro-1-methyl-pyridin-1-ium (2.34 g, 9.17 mmol) and triethylamine (2.04 mL, 14.67 mmol). After the reaction was stirred at 80° C. for 1.5 h, the mixture was purified by SiO₂ chromatography (EA/petroleum ether) to provide 585 mg (40%) of 1-(4-bromophenyl)-5-cyclopropyl-N-(2-methylpyridin-4-yl)-1H-1,2,3-triazole-4-carboxamide (INT 20C) as a yellow solid. LCMS-ESI (m/z) calculated for C₁₈H₁₆BrN₅O: 398.3; found 399.7 [M+H]⁺, t_{R}=0.484 min (Method 7). ¹H NMR (400 MHz, DMSO-D₆)=10.77 (s, 1H), 8.33 (d, J=5.5 Hz, 1H), 7.87 (d, J=8.6 Hz, 2H), 7.81 (s, 1H), 7.72-7.63 (m, 3H), 2.45 (s, 3H), 2.14-2.05 (m, 1H), 0.93 (br dd, J=2.3, 8.5 Hz, 2H), 0.78 (br dd, J=1.9, 5.4 Hz, 2H).

### Step 20-4. Synthesis of 5-cyclopropyl-1-(4-(3,5-dimethyl-1H-pyrazol-4-yl)phenyl)-N-(2-methylpyridin-4-yl)-1H-1,2,3-triazole-4-carboxamide (INT 20D)

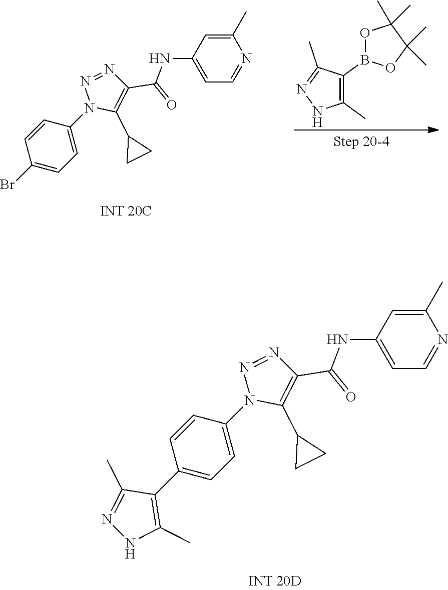 [SMILES: Cc1cc(NC(=O)c2nnn(-c3ccc(Br)cc3)c2C2CC2)ccn1 ; Cc1n[nH]c(C)c1B1OC(C)(C)C(C)(C)O1 ; Cc1cc(NC(=O)c2nnn(-c3ccc(-c4c(C)n[nH]c4C)cc3)c2C2CC2)ccn1]

To a solution of INT 20C (200 mg, 502.19 μmol) and 3,5-dimethyl-4-(4,4,5,5-tetramethyl-1,3,2-dioxaborolan-2-yl)-1H-pyrazole (167.3 mg, 753.3 μmol) in dioxane (2 mL) and H₂O (0.2 mL) were added Na₂CO₃ (159.7 mg, 1.5 mmol) and Pd(dppf)Cl₂ (36.8 mg, 50.2 μmol) under an N₂ atmosphere. The reaction was stirred at 100° C. for 1.5 h. The reaction mixture was diluted with H₂O and extracted with EA (2×). The combined organic layers were washed with brine and dried (Na₂SO₄), concentrated, and purified by SiO₂ chromatography (MeOH/DCM) to produce 120 mg (46%) of 5-cyclopropyl-1-(4-(3,5-dimethyl-1H-pyrazol-4-yl)phenyl)-N-(2-methylpyridin-4-yl)-1H-1,2,3-triazole-4-carboxamide (INT 20D) as a white solid. LCMS-ESI (m/z) calculated for C₂₃H₂₃N₇O: 413.5; found 414.2 [M+H]⁺, t_{R}=0.387 min (Method 7). ¹H NMR (400 MHz, DMSO-D₆) δ=10.76-10.72 (m, 1H), 8.32 (s, 1H), 7.79 (s, 1H), 7.71 (d, J=8.4 Hz, 2H), 7.67-7.62 (m, 1H), 7.56 (br d, J=8.5 Hz, 3H), 2.52 (br s, 6H), 2.45 (s, 3H), 2.15-2.12 (m, 1H), 0.96 (td, J=2.6, 8.5 Hz, 2H), 0.91 (td, J=2.7, 5.4 Hz, 2H).

### Step 20-5. Synthesis of 5-cyclopropyl-1-(4-(3,5-dimethyl-1-(2,2,2-trifluoroethyl)-1H-pyrazol-4-yl)phenyl)-N-(2-methylpyridin-4-yl)-1H-1,2,3-triazole-4-carboxamide

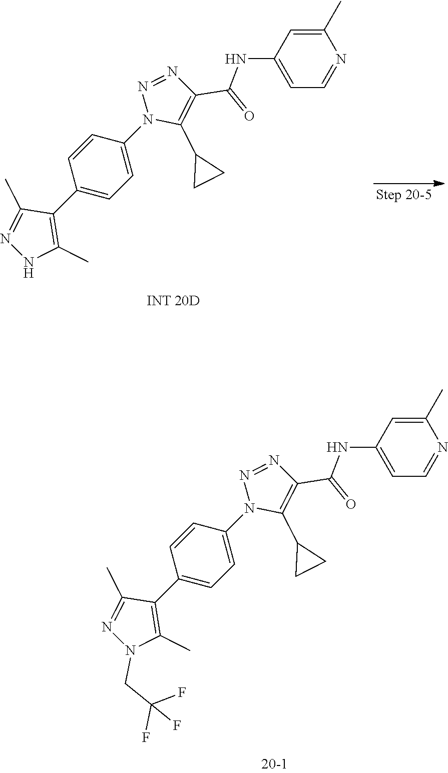 [SMILES: Cc1cc(NC(=O)c2nnn(-c3ccc(-c4c(C)n[nH]c4C)cc3)c2C2CC2)ccn1 ; Cc1cc(NC(=O)c2nnn(-c3ccc(-c4c(C)nn(CC(F)(F)F)c4C)cc3)c2C2CC2)ccn1]

To a solution of INT 20D (30 mg, 72.6 μmol) and 2,2,2-trifluoroethyl trifluoromethanesulfonate (25.3 mg, 108.8 μmol, 1.5 eq) in NMP (1 mL) was added Cs₂CO₃ (70.9 mg, 217.7 μmol). After stirring the reaction 60° C. for 2 h, the reaction mixture was purified by prep-HPLC chromatography (H₂O (FA)/CH₃CN) to provide 15.3 mg (43%) of 5-cyclopropyl-1-(4-(3,5-dimethyl-1-(2,2,2-trifluoroethyl)-1H-pyrazol-4-yl)phenyl)-N-(2-methylpyridin-4-yl)-1H-1,2,3-triazole-4-carboxamide (Compound 20-1) as an off-white solid. LCMS-ESI (m/z) calculated for C₂₅H₂₄F₃N₇O: 495.55; found 496.3 [M+H]⁺, t_{R}=0.451 min (Method 7). ¹H NMR (400 MHz, DMSO-D₆) δ=11.72 (s, 1H), 8.63 (d, J=6.8 Hz, 1H), 8.34 (d, J=1.9 Hz, 1H), 8.18 (dd, J=2.1, 6.8 Hz, 1H), 7.80-7.72 (m, 2H), 7.63-7.55 (m, 2H), 5.09 (br d, J=9.1 Hz, 2H), 2.67 (s, 3H), 2.35 (s, 3H), 2.23 (s, 3H), 2.20-2.12 (m, 1H), 1.03-0.96 (m, 2H), 0.95-0.88 (m, 2H).

The compound listed in Table 20 were made using the procedures of Scheme 20.

TABLE 20

| | | Purity | | | | |
|---|---|---|---|---|---|---|
| | Cpd | RT | | observed | | Purity |
| Structure | No. | (min) | MW | m/z | Ion | Method |
| 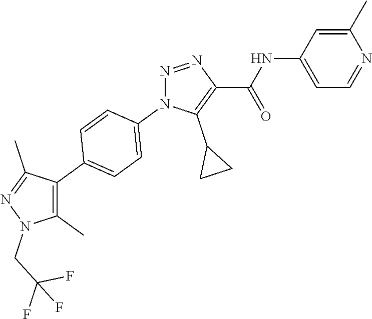 [SMILES: Cc1cc(NC(=O)c2nnn(-c3ccc(-c4c(C)nn(CC(F)(F)F)c4C)cc3)c2C2CC2)ccn1] | 20-1 | 0.451 | 495.5 | 496.3 | [M + H]⁺ | 4 |

### Example 21

### Synthesis of Compound 21-1 and Other Representative Compounds

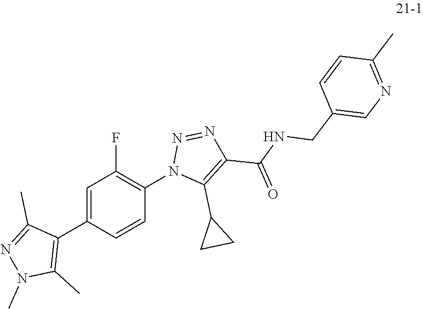 [SMILES: Cc1ccc(CNC(=O)c2nnn(-c3ccc(-c4c(C)nn(C)c4C)cc3F)c2C2CC2)cn1]

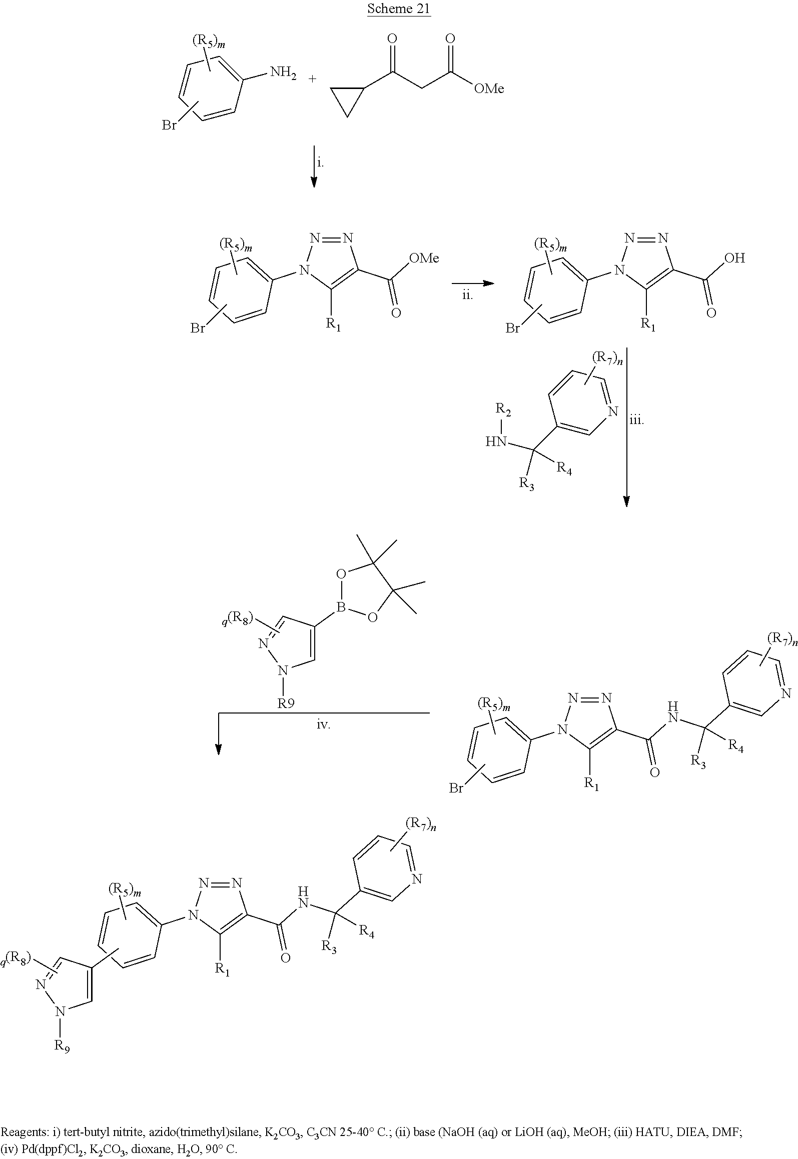

### Step 21-1. Synthesis of methyl 1-(4-bromo-2-fluorophenyl)-5-cyclopropyl-1H-1,2,3-triazole-4-carboxylate (INT 21A)

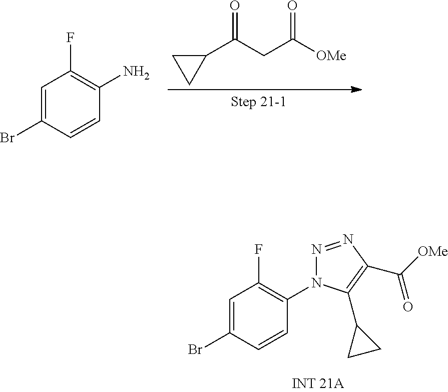 [SMILES: Nc1ccc(Br)cc1F ; COC(=O)CC(=O)C1CC1 ; COC(=O)c1nnn(-c2ccc(Br)cc2F)c1C1CC1]

To a solution of 4-bromo-2-fluoro-aniline (5 g, 26.3 mmol) in CH₃CN (200 mL) at 0° C. were added tert-butyl nitrite (4.69 mL, 39.5 mmol) and azido (trimethyl) silane (4.15 mL, 31.6 mmol). After stirring at 25° C. for 3 h, methyl 3-cyclopropyl-3-oxo-propanoate (4.49 g, 31.6 mmol) and K₂CO₃ (10.91 g, 78.9 mmol) were added, and the reaction mixture was stirred at 40° C. for 12 h. The reaction mixture was poured into saturated, aqueous NaHCO₃ solution at 0° C., and extracted with EA (3×). The combined organic layers were washed with brine (2×), dried (Na₂SO₄), filtered and concentrated under vacuum. The resulting residue was purified by SiO₂ chromatography (EA/petroleum ether) to afford 997 mg (10.1%) of methyl 1-(4-bromo-2-fluorophenyl)-5-cyclopropyl-1H-1,2,3-triazole-4-carboxylate (INT 21A) as a brown solid. LCMS-ESI (m/z) calculated for C₁₃H₁₁BrFN₃O₂: 339.0; found 339.9 [M+H]⁺, t_{R}=0.499 min (Method 7). ¹H NMR (400 MHz, DMSO-D₆) δ=8.05-7.99 (m, 1H), 7.79-7.72 (m, 2H), 3.88 (s, 3H), 1.97-1.91 (m, 1H), 0.90-0.85 (m, 2H), 0.71 (br dd, J=1.6, 5.3 Hz, 2H).

### Step 21-2. Synthesis of 1-(4-bromo-2-fluorophenyl)-5-cyclopropyl-1H-1,2,3-triazole-4-carboxylic acid (INT 21B)

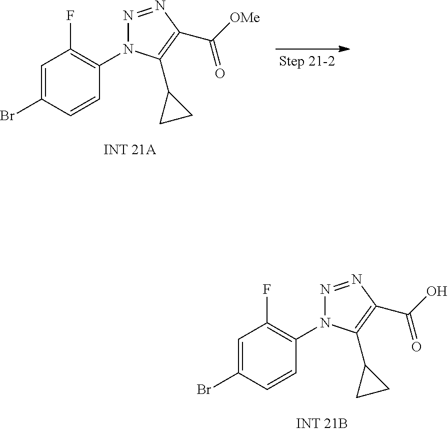 [SMILES: COC(=O)c1nnn(-c2ccc(Br)cc2F)c1C1CC1 ; O=C(O)c1nnn(-c2ccc(Br)cc2F)c1C1CC1]

To a solution of INT 21A (100 mg, 0.29 mmol) in THF (1 mL) and H₂O (1 mL) was added LiOH·H₂O (24.7 mg, 0.59 mmol). After the reaction was stirred at 25° C. for 2 h, the reaction mixture was adjusted using 1 M HCl solution to pH 6-7 and concentrated in vacuo to give 87 mg (90.7%) of 1-(4-bromo-2-fluorophenyl)-5-cyclopropyl-1H-1,2,3-triazole-4-carboxylic acid (INT 21B) of brown solid that was used without further purification in the next step. LCMS-ESI (m/z) calculated for C₁₂H₉BrFN₃O₂: 325.0; found 327.9 [M+H]⁺, t_{R}=0.442 min (Method 7).

### Step 21-3. Synthesis of 1-(4-bromo-2-fluorophenyl)-5-cyclopropyl-N-((6-methylpyridin-3-yl)methyl)-1H-1,2,3-triazole-4-carboxamide (INT 21C)

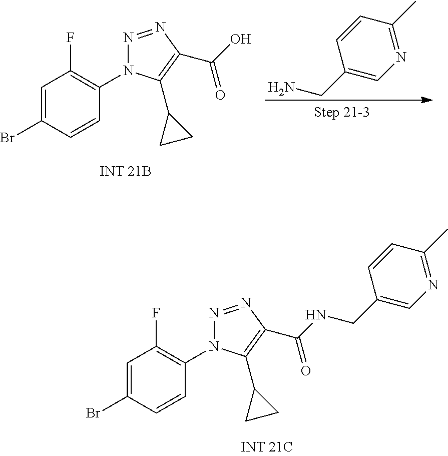

To a solution of INT 21B (87 mg, 0.27 mmol) and 2-methylpyridin-4-amine (32.6 mg, 0.27 mmol) in DMF (1 mL) were added HATU (152.2 mg, 0.40 mmol) and DIEA (139.4 μL, 0.80 mmol). After the reaction was stirred at 25° C. for 2 h, the mixture was diluted with H₂O and extracted with EA (3×). The combined organic layers were washed with brine, dried (Na₂SO₄), filtered, concentrated and purified by preparatory TLC chromatography (EA, collected peak at R_{f}=0.7) to provide 68 mg (47%) of 1-(4-bromo-2-fluorophenyl)-5-cyclopropyl-N-((6-methylpyridin-3-yl)methyl)-1H-1,2,3-triazole-4-carboxamide (INT 21C) as a brown oil. LCMS-ESI (m/z) calculated for C₁₉H₁₇BrFcN₅O: 429.1; found 430.0 [M+H]⁺, t_{R}=0.401 min (Method 7).

### Step 21-4. Synthesis of 5-cyclopropyl-1-(2-fluoro-4-(1,3,5-trimethyl-1H-pyrazol-4-yl)phenyl)-N-((6-methylpyridin-3-yl)methyl)-1H-1,2,3-triazole-4-carboxamide (Compound 21-1)

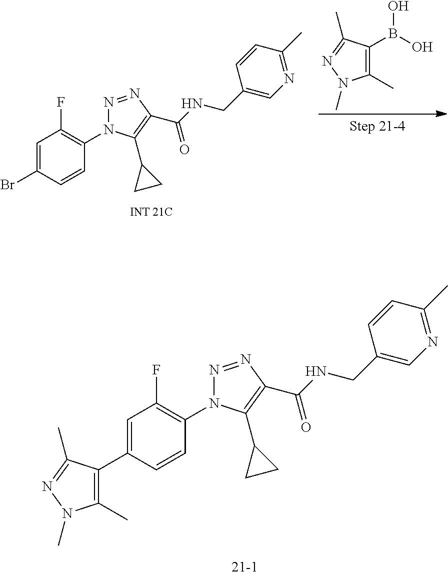

To a solution of INT 21C (68 mg, 0.16 mmol) and (1,3,5-trimethylpyrazol-4-yl) boronic acid (29.20 mg, 0.19 mmol) in dioxane (1 mL) and H₂O (0.1 mL) were added Na₂CO₃ (50.25 mg, 0.47 mmol) and Pd(dppf)Cl₂ (11.6 mg, 0.016 mmol) under an N₂ atmosphere. The reaction was stirred at 100° C. for 12 h. The reaction mixture was diluted with 10 mL MeOH, then 30 mg thiourea resin was added and the mixture was stirred at 25° C. for 4 hr. The residue was purified by prep-HPLC chromatography (H₂O (FA)-CH₃CN) to produce 22.6 mg (31.1%) of 5-cyclopropyl-1-(2-fluoro-4-(1,3,5-trimethyl-1H-pyrazol-4-yl) phenyl)-N-((6-methylpyridin-3-yl) methyl)-1H-1,2,3-triazole-4-carboxamide (Compound 21-1) as a white solid. LCMS-ESI (m/z) calculated for C₂₅H₂₆FN₇O: 459.2; found 460.3 [M+H]⁺, t_{R}=0.39 min (Method 7). ¹H NMR (400 MHz, DMSO-D₆) δ=9.19 (br t, J=6.1 Hz, 1H), 8.42 (s, 1H), 7.75 (t, J=8.1 Hz, 1H), 7.63 (dd, J=1.8, 7.8 Hz, 1H), 7.47 (br d, J=11.6 Hz, 1H), 7.35 (br d, J=8.1 Hz, 1H), 7.21 (d, J=8.0 Hz, 1H), 4.43 (br d, J=6.0 Hz, 2H), 3.73 (s, 3H), 2.44 (s, 3H), 2.30 (s, 3H), 2.21 (s, 3H), 2.01-1.86 (m, 1H), 1.00-0.94 (m, 2H), 0.92-0.84 (m, 2H).

The compounds listed in Table 21 were made using the procedures of Scheme 21.

TABLE 21

| | | Purity | | | | |
|---|---|---|---|---|---|---|
| | Cpd | RT | | observed | | Purity |
| Structure | No. | (min) | MW | m/z | Ion | Method |
| 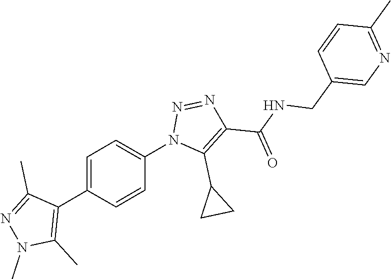 [SMILES: Cc1ccc(CNC(=O)c2nnn(-c3ccc(-c4c(C)nn(C)c4C)cc3)c2C2CC2)cn1] | 21-1 | 0.39 | 459.5 | 460.3 | [M + H]⁺ | 7 |
| 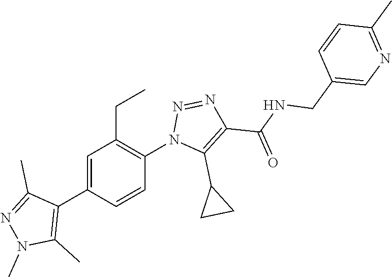 [SMILES: CCc1cc(-c2c(C)nn(C)c2C)ccc1-n1nnc(C(=O)NCc2ccc(C)nc2)c1C1CC1] | 21-2 | 0.381 | 471.6 | 472.4 | [M + H]⁺ | 7 |

### Example 22

### Synthesis of Compound 22-1 and Other Representative Compounds

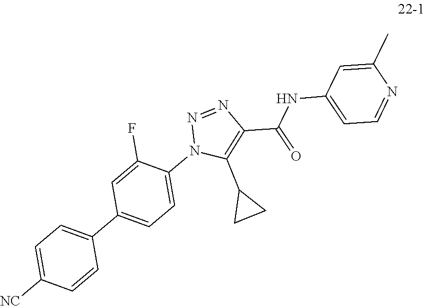 [SMILES: Cc1cc(NC(=O)c2nnn(-c3ccc(-c4ccc(C#N)cc4)cc3F)c2C2CC2)ccn1]

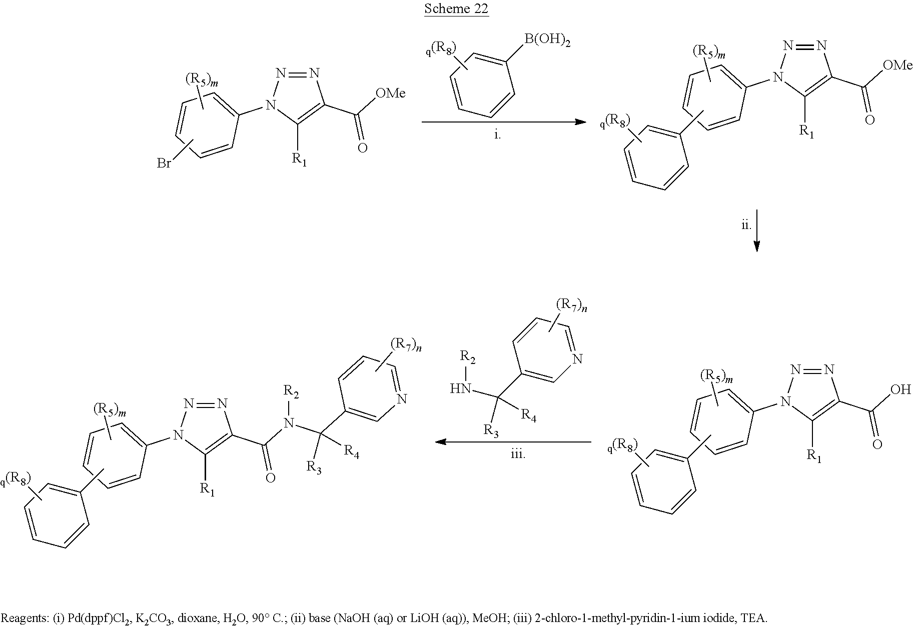

### Step 22-1. Synthesis of methyl 1-(4′-cyano-3-fluoro-[1,1′-biphenyl]-4-yl)-S-cyclopropyl -1H-1,2,3-triazole-4-carboxylate (INT 22A)

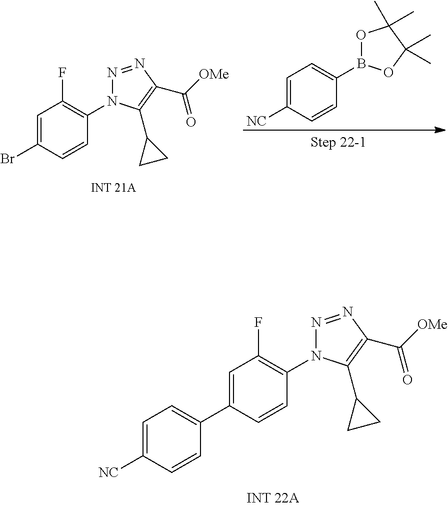 [SMILES: COC(=O)c1nnn(-c2ccc(Br)cc2F)c1C1CC1 ; CC1(C)OB(c2ccc(C#N)cc2)OC1(C)C ; COC(=O)c1nnn(-c2ccc(-c3ccc(C#N)cc3)cc2F)c1C1CC1]

To a solution of INT 21A (800 mg, 2.35 mmol) and 4-(4,4,5,5-tetramethyl-1,3,2-dioxaborolan-2-yl) benzonitrile (538.8 mg, 2.35 mmol) in dioxane (15 mL) and H₂O (1.5 mL) were added Na₂CO₃ (747.8 mg, 7.06 mmol) and Pd(dppf)Cl₂ (172.1 mg, 0.24 mmol). The reaction was stirred at 100° C. for 12 h. The reaction mixture was diluted with H₂O and extracted with EA (3×). The combined organic layers were washed with brine, dried (Na₂SO₄), filtered, and concentrated to provide a residue that purified by SiO₂ chromatography (EA/petroleum ether) to produce 450 mg (52.8%) of methyl 1-(4′-cyano-3-fluoro-[1,1′-biphenyl]-4-yl)-5-cyclopropyl-1H-1,2,3-triazole-4-carboxylate (INT 22A) as a yellow solid. LCMS-ESI (m/z) calculated for C₂₀H₁₅FN₄O₂: 362.4; found 363.0 [M+H]⁺, t_{R}=044 min (Method 7). ¹H NMR (400 MHz, DMSO-D₆) δ=8.04-8.12 (m, 3H), 8.00-8.04 (m, 2H), 7.91 (d, J=4.00 Hz, 2H), 3.89 (s, 3H), 1.95-2.05 (m, 1H), 0.88-0.96 (m, 2H), 0.73-0.80 (m, 2H).

### Step 22-2. Synthesis of 1-(4′-cyano-3-fluoro-[1,1′-biphenyl]-4-yl)-5-cyclopropyl-1H-1,2,3-triazole-4-carboxylic acid (INT 22B)

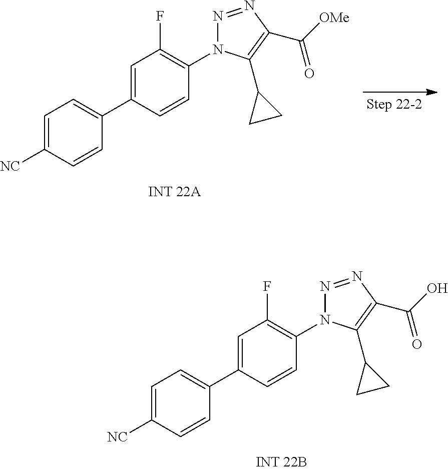 [SMILES: COC(=O)c1nnn(-c2ccc(-c3ccc(C#N)cc3)cc2F)c1C1CC1 ; N#Cc1ccc(-c2ccc(-n3nnc(C(=O)O)c3C3CC3)c(F)c2)cc1]

To a solution of INT 22A (400 mg, 1.1 mmol) in THF (2 mL) and H₂O (2 mL) was added LiOH H₂O (139 mg, 3.31 mmol). After the reaction was stirred at 25° C. for 4 h, the reaction mixture was adjusted using 1 M HCl solution to pH 7 and concentrated in vacuo to give a residue that was purified by prep HPLC chromatography (H₂O (FA)/CH₃CN) to provide 300 mg (78%) of 1-(4′-cyano-3-fluoro-[1,1′-biphenyl]-4-yl)-5-cyclopropyl-1H-1,2,3-triazole-4-carboxylic acid (INT 22B) of white solid. LCMS-ESI (m/z) calculated for C₁₉H₁₃FN₄O₂: 348.3; found 349.2 [M+H]⁺, t_{R}=0.522 min (Method 7). ¹H NMR (400 MHz, DMSO-D₆) δ=8.05-8.11 (m, 3H), 7.99-8.03 (m, 2H), 7.89-7.92 (m, 2H), 1.96-2.05 (m, 1H), 0.86-0.94 (m, 2H), 0.74-0.81 (m, 2H).

### Step 22-3. Synthesis of 1-(4′-cyano-3-fluoro-[1,1′-biphenyl]-4-yl)-5-cyclopropyl-N-(2-methylpyridin-4-yl)-1H-1,2,3-triazole-4-carboxamide (Compound 22-1)

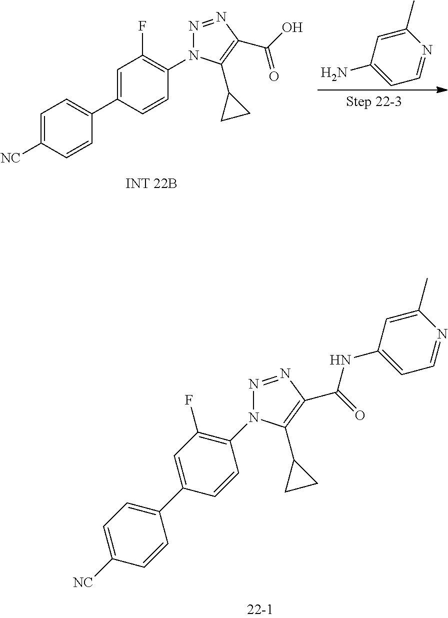

To a solution of INT 22B (50 mg, 0.14 mmol) and 2-methylpyridin-4-amine (18.6 mg, 0.17 mmol) in DCE (2 mL) were added 2-chloro-1-methyl-pyridin-1-ium iodide (110 mg, 0.43 mmol) and TEA (50 μL, 0.36 mmol). After the reaction was stirred at 83° C. for 2 h, the mixture was filtered, concentrated, and purified by prep-HPLC chromatography (H₂O (FA)/CH₃CN) to provide 24.6 mg (39%) of 1-(4′-cyano-3-fluoro-[1,1′-biphenyl]-4-yl)-5-cyclopropyl-N-(2-methylpyridin-4-yl)-1H-1,2,3-triazole-4-carboxamide (Compound 22-1) as a yellow solid. LCMS-ESI (m/z) calculated for C₂₅H₁₉FN₆O: 438.5; found 439.2 [M+H]⁺, t_{R}˜=0.48 min (Method 7). ¹H NMR (400 MHz, DMSO-D₆) δ=10.92 (s, 1H), 8.37 (d, J=5.75 Hz, 1H), 8.05-8.12 (m, 3H), 8.00-8.04 (m, 2H), 7.89-7.98 (m, 2H), 7.85 (s, 1H), 7.71 (br d, J=5.63 Hz, 1H), 2.47 (s, 3H), 2.02 (quin, J=6.97 Hz, 1H), 0.92-1.00 (in, 4H).

The compounds listed in Table 22 were made using the procedures of Scheme 22.

TABLE 22

| | | Purity | | | | |
|---|---|---|---|---|---|---|
| | Cpd | RT | | observed | | Purity |
| Structure | No. | (min) | MW | m/z | Ion | Method |
| 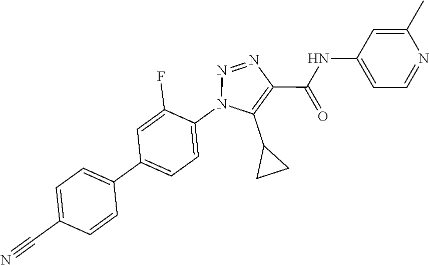 [SMILES: Cc1cc(NC(=O)c2nnn(-c3ccc(-c4ccc(C#N)cc4)cc3F)c2C2CC2)ccn1] | 22-1 | 0.475 | 438.5 | 439.2 | [M + H]⁺ | 4 |
| 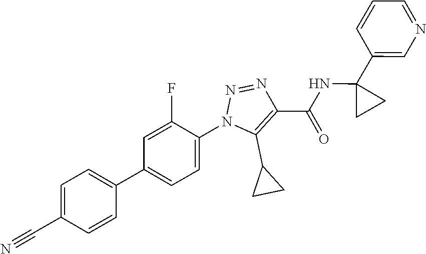 [SMILES: N#Cc1ccc(-c2ccc(-n3nnc(C(=O)NC4(c5cccnc5)CC4)c3C3CC3)c(F)c2)cc1] | 22-2 | 0.462 | 464.5 | 465.2 | [M + H]⁺ | 4 |
| 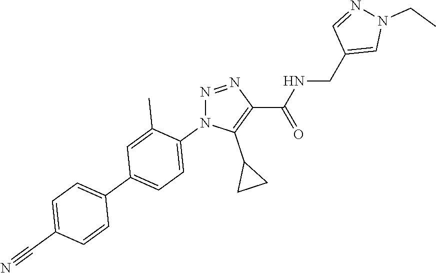 | 22-3 | 0.569 | 455.5 | 456.1 | [M + H]⁺ | 4 |
| 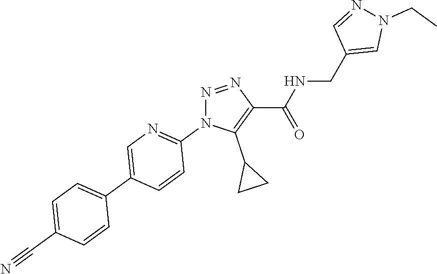 [SMILES: CCn1cc(CNC(=O)c2nnn(-c3ccc(-c4ccc(C#N)cc4)cn3)c2C2CC2)cn1] | 22-4 | 0.466 | 438.5 | 439.2 | [M + H]⁺ | 4 |

### Example 23

### Synthesis of Compound 23-1 and Other Representative Compounds

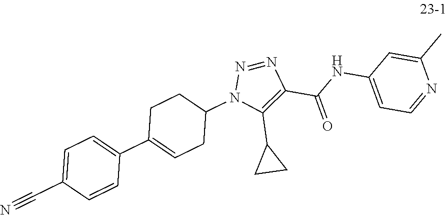 [SMILES: Cc1cc(NC(=O)c2nnn(C3CC=C(c4ccc(C#N)cc4)CC3)c2C2CC2)ccn1]

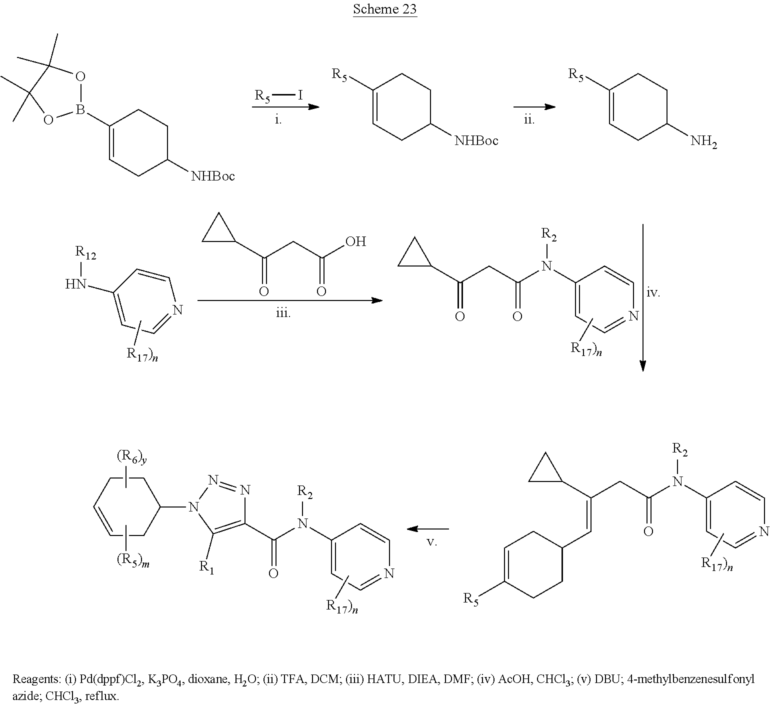

Step 23-1. Synthesis of tert-butyl (4′-cyano-2,3,4,5-tetrahydro-[1,1′-biphenyl]-4-yl)carbamate (INT 23A)

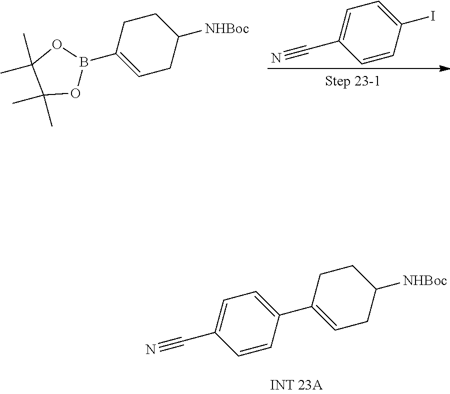 [SMILES: CC(C)(C)OC(=O)NC1CC=C(B2OC(C)(C)C(C)(C)O2)CC1 ; N#Cc1ccc(I)cc1 ; CC(C)(C)OC(=O)NC1CC=C(c2ccc(C#N)cc2)CC1]

To a solution of ENT 21A tert-butyl (4-(4,4,5,5-tetramethyl-1,3,2-dioxaborolan-2-yl)cyclohex-3-en-1-yl)carbamate (200 mg, 0.62 mmol) and 4-iodobenzonitrile (170 mg, 0.74 mmol) in dioxane (2 mL) and H₂O (2 mL) were added K₃PO₄ (394 mg, 1.9 mmol) and Pd(dppf)Cl₂ (45 mg, 0.06 mmol). The reaction was stirred at 120° C. for 2 h. The reaction mixture was cooled to rt diluted with EA and washed with saturated aqueous NaHCO₃ and brine, then dried (Na₂SO₄), filtered, and concentrated to provide a residue that purified by SiO₂ chromatography (MeOH/DCM) to produce 165 mg (89.4%) of tert-butyl (4′-cyano-2,3,4,5-tetrahydro-[1,1′-biphenyl]-4-yl) carbamate (INT 23A). LCMS-ESI (m/z) calculated for C₁₈H₂₂N₂O₂: 298.4; found 299.2 [M+H]⁺, t_{R}=6.32 min (Method 3).

### Step 23-2. Synthesis of 4′-amino-2′,3′,4′,5′-tetrahydro-[1,1′-biphenyl]-4-carbonitrile (INT 23B)

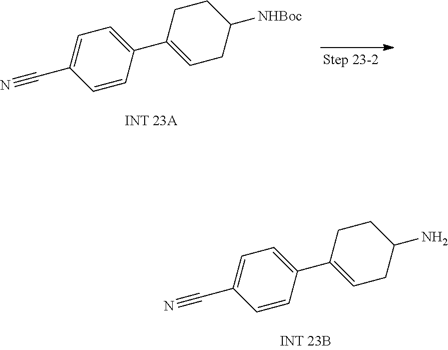 [SMILES: CC(C)(C)OC(=O)NC1CC=C(c2ccc(C#N)cc2)CC1 ; N#Cc1ccc(C2=CCC(N)CC2)cc1]

To a solution of INT 23A (165 mg, 0.55 mmol) in DCM (10 mL) was added TFA (1 mL). After the reaction was stirred at it for 16 h, the reaction mixture was concentrated in vacuo to give a residue that was dissolved in DCM, washed with 1M NaOH and brine, then dried (Na₂SO₄), filtered and concentrated to provide 97 mg (89%) of 4′-amino-2′,3′,4′,5′-tetrahydro-[1,1′-biphenyl]-4-carbonitrile (INT 23B) that was used without further purification. LCMS-ESI (m/z) calculated for C₁₃H₁₄N₂: 198.3; found 199.1 [M+H]⁺, t_{R}=4.92 min (Method 3).

### Step 23-3. Synthesis of 3-cyclopropyl-N-(2-methylpyridin-4-yl)-3-oxopropanamide (INT 23C)

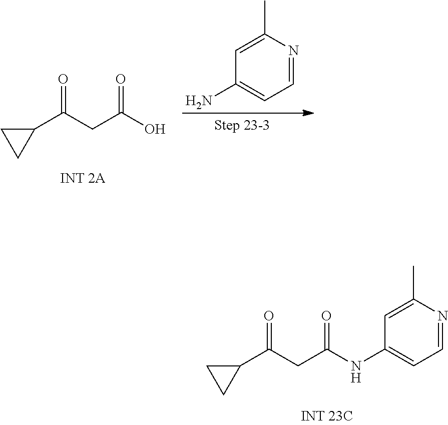

To a solution of INT 2A (1.7 g, 13.8 mmol) and 2-methylpyridin-4-amine (1.43 g, 15.2 mmol) in DMF (10 mL) were added HATU (5.79 g, 15.2 mmol) and DIEA (5.35 g, 41.4 mmol). After the reaction was stirred at rt for 12 h, the mixture was diluted with EA, washed with H₂O, saturated aqueous NaHCO₃, H₂O, and brine, then dried (Na₂SO₄), concentrated, and purified by SiO₂ chromatography (EA/hexanes) to provide 2.5 g (83%) of 3-cyclopropyl-N-(2-methylpyridin-4-yl)-3-oxopropanamide (INT 23C). LCMS-ESI (m/z) calculated for C₁₂H₁₄N₂O₂: 218.2; found 219.0 [M+H]⁺. (Method 3). ¹H NMR (400 MHz, CDCl₃) δ 10.47 (s, 1H), 8.35 (d, J=4.0 Hz, 1H), 7.70 (s, 1H), 7.58 (d, J=4.0 Hz, 1H), 3.85 (s, 2H), 2.67 (s, 3H), 2.03-2.09 (m, 1H), 1.17-1.25 (m, 2H), 1.09-1.14 (m, 2H).

### Step 23-4. Synthesis of 3-((4′-cyano-2,3,4,5-tetrahydro-[1,1′-biphenyl]-4-yl)imino)-3-cyclopropyl-N-(2-methylpyridin-4-yl)propanamide (INT 23D)

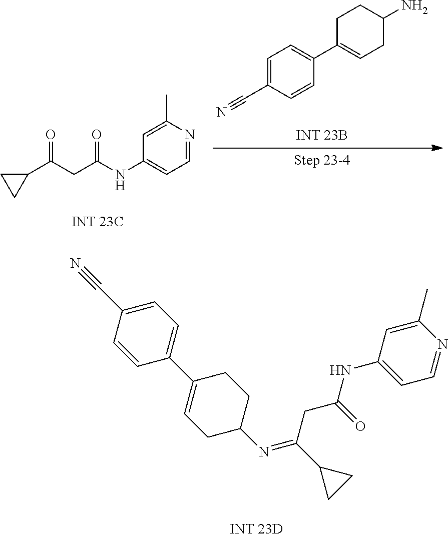

To a solution of INT 23C (71 mg, 0.33 mmol) and INT 23B (95 mg, 0.36 mmol) in CHCl₃ (20 mL) was added AcOH (58 mg, 0.97 mmol). After stirring at reflux for 36 h, the mixture was diluted with DCM, washed with saturated aqueous NaHCO₃, and brine, then dried (Na₂SO₄), filtered and concentrated to provide 129 mg of crude of 3-((4′-cyano-2,3,4,5-tetrahydro-[1,1′-biphenyl]-4-yl)imino)-3-cyclopropyl-N-(2-methyl pyridin-4-yl)propanamide (INT 23D) that was used in the next step without any further purification. LCMS-ESI (m/z) calculated for C₂₅H₂₆N₄O: 398.5; found 398.9 [M+H]⁺, t_{R}=5.95 min (Method 3).

### Step 23-5. Synthesis of 1-(4′-cyano-2,3,4,5-tetrahydro-[1,1′-biphenyl]-4-yl)-5-cyclopropyl-N-(2-methylpyridin-4-yl)-1H-1,2,3-triazole-4-carboxamide (Compound 23-1)

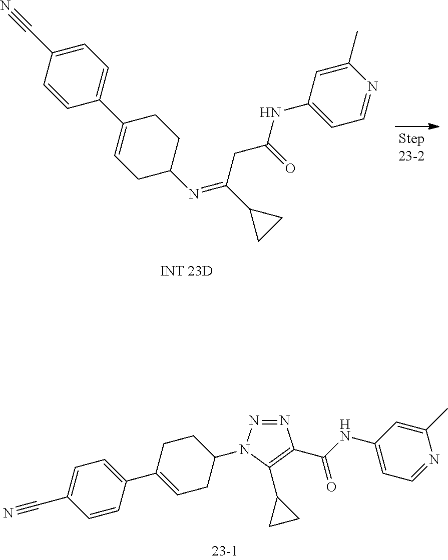 [SMILES: Cc1cc(NC(=O)C/C(=N\C2CC=C(c3ccc(C#N)cc3)CC2)C2CC2)ccn1 ; Cc1cc(NC(=O)c2nnn(C3CC=C(c4ccc(C#N)cc4)CC3)c2C2CC2)ccn1]

To a solution of crude INT 23D (129 mg, 0.32 mmol) and 2-methylpyridin-4-amine (1.43 g, 15.2 mmol) in CH₃CN (10 mL) were added DBU (49 mg, 0.32 mmol) and tosyl azide (213 mg, 0.32 mmol). After the reaction was stirred at rt for 30 min, then heated to reflux for 2.5 h. Additional tosyl azide (213 mg, 0.32 mmol) was added and the mixture was continued to stir at reflux for 19 h. The mixture was cooled, diluted with EA and washed with saturated aqueous NaHCO₃ and brine, then dried (Na₂SO₄), concentrated, and purified by SiO₂ chromatography (EA/hexanes then MeOH/DCM) to provide material that was further purified by reverse-phase C-18 chromatography (MeOH/H₂O) to provide 8 mg (5.8%) of 1-(4′-cyano-2,3,4,5-tetrahydro-[1,1′-biphenyl]-4-yl)-5-cyclopropyl-N-(2-methylpyridin-4-yl)-1H-1,2,3-triazole-4-carboxamide (Compound 23-1). LCMS-ESI (m/z) calculated for C₂₅H₂₄N₆O: 424.5; found 424.6 [M+H]⁺, t_{R}=10 min (Method 2). ¹H NMR (400 MHz, DMSO-D₆) δ 10.59 (s, 1H), 8.30 (s, 1H), 7.83 (d, J=8.0 Hz, 2H), 7.81 (d, J=8.0 Hz, 2H), 7.69 (d, J=4.0 Hz, 1H), 7.59 (d, J=4.0 Hz, 1H), 6.45 (s, 1H), 4.97 (m, 1H), 2.86 (m, 2H), 2.68 (m, 2H), 2.49 (s, 3H), 2.43 (m, 2H), 2.07 (m, 1H), 1.12 (m, 2H), 1.02 (m, 2H).

The compounds listed in Table 23 were made using the procedures of Scheme 23.

TABLE 23

| | | Purity | | | | |
|---|---|---|---|---|---|---|
| | Cpd | RT | | observed | | Purity |
| Structure | No. | (min) | MW | m/z | Ion | Method |
| 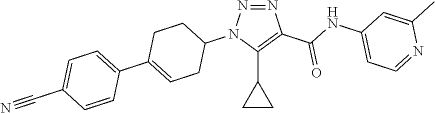 [SMILES: Cc1cc(NC(=O)c2nnn(C3CC=C(c4ccc(C#N)cc4)CC3)c2C2CC2)ccn1] | 23-1 | 11.12 | 424.5 | 424.6 | [M + H]⁺ | 2 |

### Example 24

### Synthesis of Compound 24-1 and Other Representative Compounds

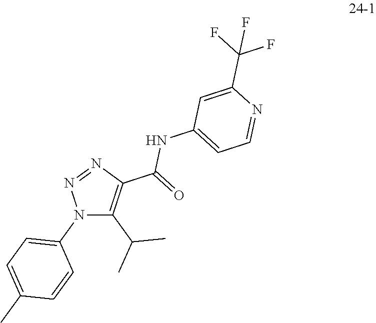 [SMILES: Cc1ccc(-n2nnc(C(=O)Nc3ccnc(C(F)(F)F)c3)c2C(C)C)cc1]

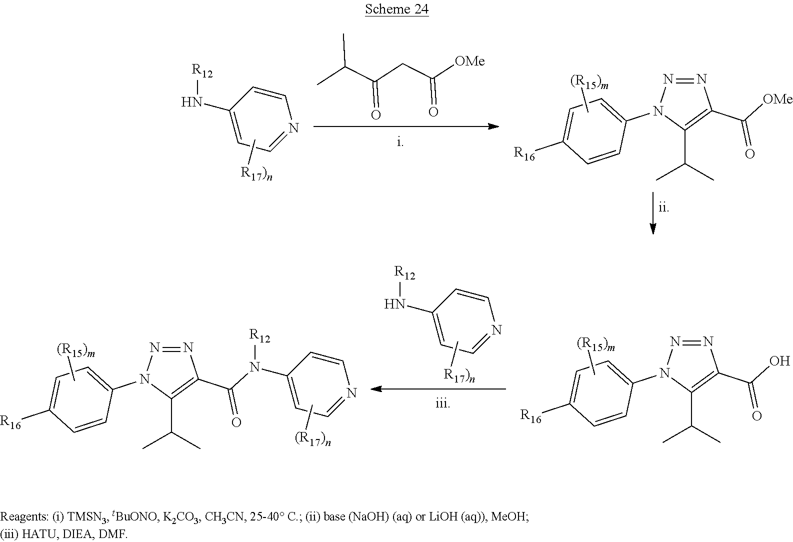

### Step 24-1. Synthesis of methyl 5-isopropyl-1-(p-tolyl)-1H-1,2,3-triazole-4-carboxylate (INT 24A)

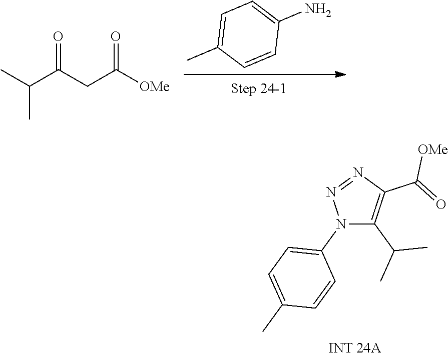

To a solution of 4-methylaniline (2.06 mL, 18.66 mmol) in CH₃CN (40 mL) were added TMSN₃ (2.95 mL, 22.40 mmol) and ^{t}BuONO (3.33 mL, 28.00 mmol) at 0° C. under N₂. After the reaction mixture was stirred at 25° C. for 2 hr, K₂CO₃ (7.74 g, 55.99 mmol) and methyl 4-methyl-3-oxo-pentanoate (3.20 mL, 22.40 mmol) were added and the mixture was further stirred at 40° C. for 12 hr. The reaction mixture was poured into saturated aqueous NaHCO₃ and extracted with EA (2×). The combined organic layers were washed with brine, dried (Na₂SO₄), filtered, and concentrated to provide a residue that purified by SiO₂ chromatography (EA/petroleum ether) to produce 4.4 g (88.6%) of methyl 5-isopropyl-1-(p-tolyl)-1H-1,2,3-triazole-4-carboxylate (INT 24A). LCMS-ESI (m/z) calculated for C₁₄H₁₇N₃O₂: 259.3; found 260.0 [M+H]⁺, t_{R}=0.55 min (Method 7). ¹H NMR (400 MHz, DMSO-D₆) δ=7.47-7.41 (m, 4H), 3.89 (s, 3H), 3.19 (spt, J=7.1 Hz, 1H), 2.43 (s, 3H), 1.24 (d, J=7.1 Hz, 6H).

### Step 24-2. Synthesis of 5-isopropyl-1-(p-tolyl)-1H-1,2,3-triazole-4-carboxylic acid (INT 24B)

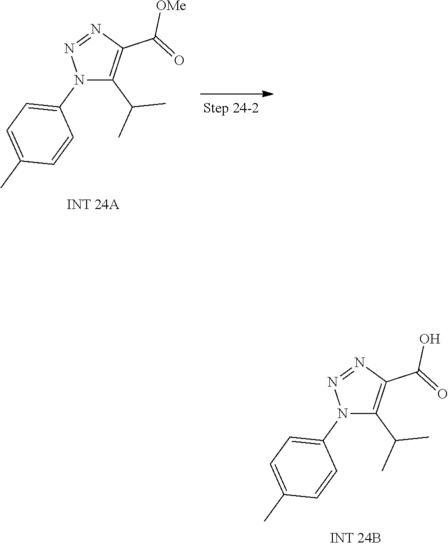 [SMILES: COC(=O)c1nnn(-c2ccc(C)cc2)c1C(C)C ; Cc1ccc(-n2nnc(C(=O)O)c2C(C)C)cc1]

To a solution of INT 24A (200 mg, 0.75 mmol) in H₂O (1 mL) and MeOH (1 mL) was added LiOH (126 mg, 3.01 mmol). After the reaction was stirred at 25° C. for 12 h, the reaction mixture was pH adjusted to pH=7 by the addition of 1M HCl. The resulting mixture was concentrated to provide 184 mg (99%) of crude 5-isopropyl-1-(p-tolyl)-1H-1,2,3-triazole-4-carboxylic acid (INT 24B) as a yellow solid that was used without further purification. LCMS-ESI (m/z) calculated for C₁₃H₁₅N₃O₂: 245.3; found 246.3 [M+H]⁺, t_{R}=4.74 min (Method 7).

### Step 24-3. Synthesis of 5-isopropyl-1-(p-tolyl)-N-(2-(trifluoromethyl)pyridin-4-yl-1H-1,2,3-triazole-4-carboxamide (Compound 24-1)

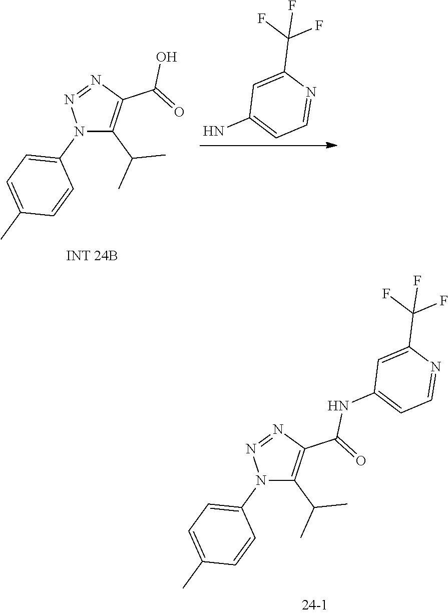

To a solution of INT 24B (184 mg, 0.75 mmol) and 2-(trifluoromethyl)pyridin-4-amine (121.6 mg, 0.75 mmol) in DCE (5 mL) were added 2-chloro-1-methyl-pyridin-1-ium-iodide (479.14 mg, 1.88 mmol) and TEA (261.04 uL, 1.88 mmol). After the reaction was stirred at 80° C. for 2 h, the mixture was concentrated, dissolved in DMF and filtered through a filter membrane. The filtrate was purified by prep HPLC (H₂O (FA)/CH₃CN) and lyophilized to provide 100.6 mg (34.4%) of 5-isopropyl-1-(p-tolyl)-N-(2-(trifluoromethyl)pyridin-4-yl)-1H-1,2,3-triazole-4-carboxamide (Compound 24-1) as a yellow solid. LCMS-ESI (m/z) calculated for C₁₉H₁₈F₃NSO₂: 389.4; found 390.1 [M+H]⁺, t_{R}=0.61 min (Method 7) ¹H NMR (400 MHz, DMSO-D₆) δ=11.34 (s, 1H), 8.67 (d, J=5.5 Hz, 1H), 8.48 (d, J=1.8 Hz, 1H), 8.19 (dd, J=1.8, 5.4 Hz, 1H), 7.48 (s, 4H), 3.23 (td, J=6.9, 14.0 Hz, 1H), 2.45 (s, 3H), 1.31 (d, J=7.0 Hz, 6H).

The compound listed in Table 24 were made using the procedures of Scheme 24.

TABLE 24

| | | Purity | | | | |
|---|---|---|---|---|---|---|
| | Cpd | RT | | observed | | Purity |
| Structure | No. | (min) | MW | m/z | Ion | Method |
| 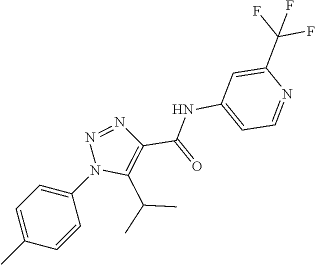 [SMILES: Cc1ccc(-n2nnc(C(=O)Nc3ccnc(C(F)(F)F)c3)c2C(C)C)cc1] | 24-1 | 0.631 | 389.4 | 390.1 | [M + H]⁺ | 7 |

### Example 25

### Synthesis of Compound 25-1 and Other Representative Compounds

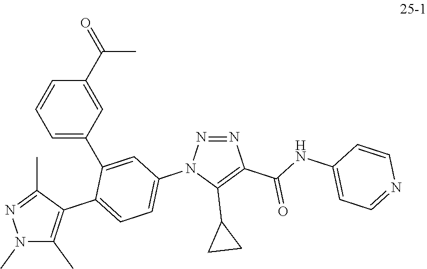 [SMILES: CC(=O)c1cccc(-c2cc(-n3nnc(C(=O)Nc4ccncc4)c3C3CC3)ccc2-c2c(C)nn(C)c2C)c1]

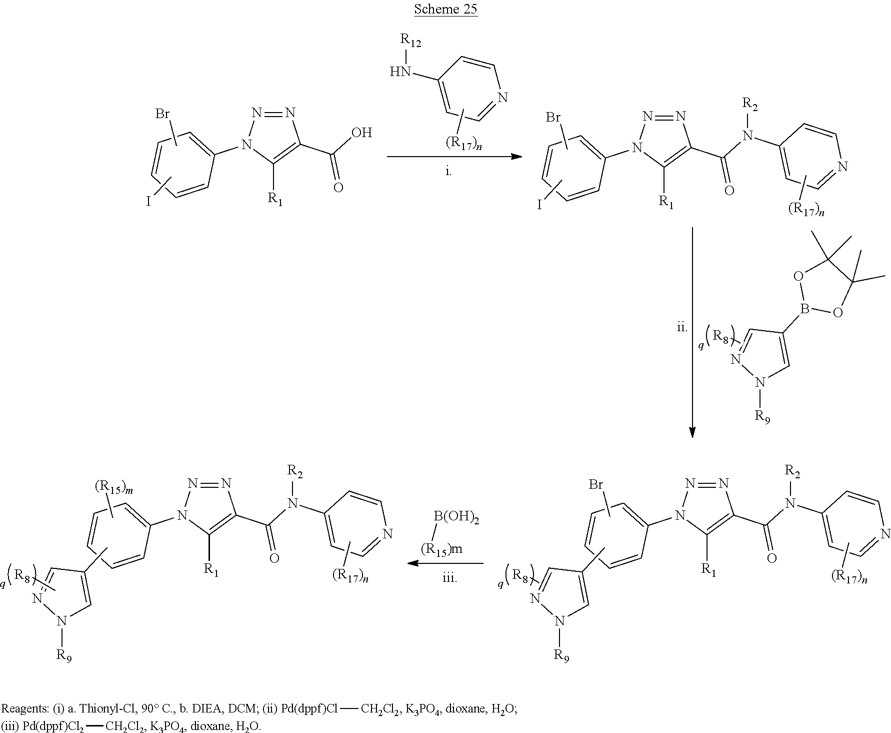

### Step 25-1. Synthesis of 1-(3-bromo-4-iodophenyl)-5-cyclopropyl-N-(pyridin-4-yl)-1H-1,2,3-triazole-4-carboxamide (INT 25A)

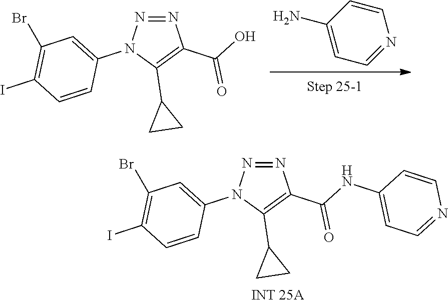

A solution of 1-(3-bromo-4-iodophenyl)-5-cyclopropyl-1H-1,2,3-triazole-4-carboxylic acid (1.0 g, 2.3 mmol) in thionyl-chloride (335 μL, 4.6 mmol) was heated at 90° C. for 30 min. The mixture was cooled, concentrated, and dissolved in DCM (10 mL). DIEA (891.6 mg, 6.9 mmol) and pyridin-4-amine (238.5 mg, 2.53 mmol) were added. The reaction mixture was stirred for 2 h then diluted with H₂O. The organic layer was isolated, concentrated and purified by SiO₂ chromatography (MeOH/DCM) to produce 880 mg (75%) of 1-(3-bromo-4-iodophenyl)-5-cyclopropyl-N-(pyridin-4-yl)-1H-1,2,3-triazole-4-carboxamide (INT 25A). LCMS-ESI (m/z) calculated for C₁₇H₁₃BrIN₅O: 508.9; found 510.0 [M+H]⁺, t_{R}=10.88 min (Method 3). ¹H NMR (400 MHz, DMSO-D₆) δ=(s, 1H), 8.47 (d, J=8 Hz, 2H), 8.22 (d, J=8 Hz, 1H), 8.14 (s, 1H), 7.87 (d, J=8 Hz, 2H), 7.51 (d, J=8 Hz, 1H), 2.12 (m, 1H), 0.93 (m, 2H), 0.78 (m, 2H).

### Step 25-2. Synthesis of 1-(3-bromo-4-(1,3,5-trimethyl-1H-pyrazol-4-yl)phenyl)-5-cyclopropyl-N-(pyridin-4-yl)-1H-1,2,3-triazole-4-carboxamide (INT 25B)

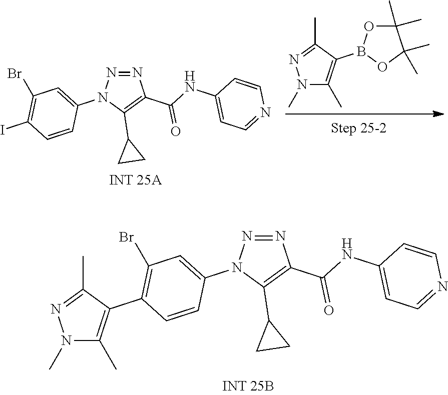

INT 25A (250 mg, 0.49 mmol), 1,3,5-trimethyl-4-(4,4,5,5-tetramethyl-1,3,2-dioxaborolan-2-yl)-1H-pyrazole (104.5 mg, 0.49 mmol), Pd(dppf)Cl₂·CH₂Cl₂ (40 mg, 0.05 mmol), and K₃PO₄ (312.2 mg, 1.47 mmol) were added to a vial, followed by dioxane (4.0 mL) and H₂O (1.5 mL). The mixture was degassed by N₂ bubbling then heated at 110° C. for 12 h. The organic layer was separated from the aqueous layer and purified by SiO₂ chromatography (MeOH/EA/hexanes) to provide 87 mg (36%) of 1-(3-bromo-4-(1,3,5-trimethyl-1H-pyrazol-4-yl)phenyl)-5-cyclopropyl-N-(pyridin-4-yl)-1H-1,2,3-triazole-4-carboxamide (INT 25B) as a tan solid. LCMS-ESI (m/z) calculated for C₂₃H₂₂BrN₇O: 491.1; found 492.5 [M+H]⁺, t_{R}=5.62 min (Method 3).

### Step 25-3. Synthesis of 1-(3′-acetyl-6-(1,3,5-trimethyl-1H-pyrazol-4-yl)-[1,1′-biphenyl]-3-yl)-5-cyclopropyl-N-(pyridin-4-yl)-1H-1,2,3-triazole-4-carboxamide (Compound 25-1)

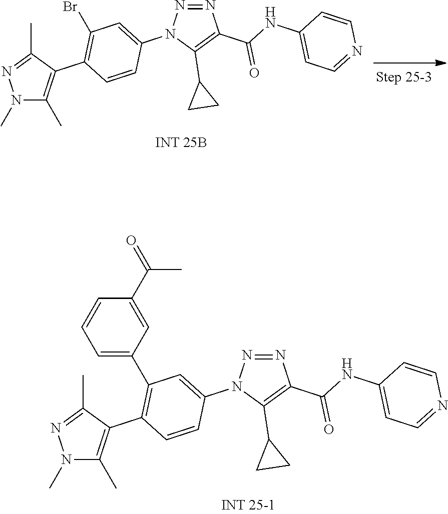 [SMILES: Cc1nn(C)c(C)c1-c1ccc(-n2nnc(C(=O)Nc3ccncc3)c2C2CC2)cc1Br ; CC(=O)c1cccc(-c2cc(-n3nnc(C(=O)Nc4ccncc4)c3C3CC3)ccc2-c2c(C)nn(C)c2C)c1]

INT 25B (41 mg, 0.08 mmol), (3-acetylphenyl)boronic acid (20.5 mg, 0.13 mmol), Pd(dppf)Cl₂·CH₂Cl₂ (6.8 mg, 0.008 mmol), and K₃PO₄ (53.07 mg, 0.25 mmol) were added to a vial, followed by dioxane (1.0 mL) and H₂O (0.5 mL). The organic layer was separated from the aqueous and purified by SiO₂ chromatography (MeOH/EA/hexanes) to provide 24.6 mg (55%) of 1-(3′-acetyl-6-(1,3,5-trimethyl-1H-pyrazol-4-yl)-[1,1′-biphenyl]-3-yl)-5-cyclopropyl-N-(pyridin-4-yl)-1H-1,2,3-triazole-4-carboxamide (Compound 25-1) as a tan solid. LCMS-ESI (m/z) calculated for C₃₁H₂₉N₇O₂: 531.2; found 532 [M+H]⁺, t_{R}=10.6 min (Method 7).

The compounds listed in Table 25 were made using the procedures of Scheme 25.

TABLE 25

| | | Purity | | | | |
|---|---|---|---|---|---|---|
| | Cpd | RT | | observed | | Purity |
| Structure | No. | (min) | MW | m/z | Ion | Method |
| 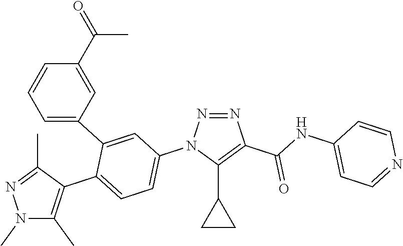 [SMILES: CC(=O)c1cccc(-c2cc(-n3nnc(C(=O)Nc4ccncc4)c3C3CC3)ccc2-c2c(C)nn(C)c2C)c1] | 25-1 | 10.93 | 531.6 | 531.8 | [M + H]⁺ | 2 |
| 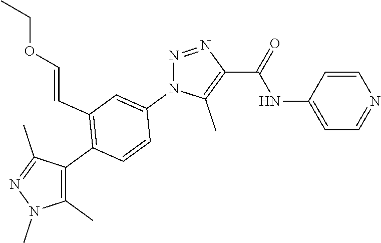 | 25-2 | 11.19 | 483.6 | 484.3 | [M + H]⁺ | 2 |

### Example 26

### MRGPRD Activity

Chinese Hamster Ovary (CHO) cells stably expressing human MRGPRD (Eurofins, San Diego CA) were plated in a 384-well plate at 20,000 cells per well in 12 μL of Optimem (Fisher Scientific 11-058-021). Plates were kept in the incubator at 37° C. overnight. Antagonists were solubilized at a concentration of 10 mM in DMSO, and the agonists were solubilized to stock concentrations of either 50 mM in H₂O (β-Alanine) or 10 mM in DMSO (all others) and then diluted in assay buffer (final concentrations of 5.7 mM Tris-HCl, 43 mM NaCl, 50 mM LiCl, pH=8). The concentration of DMSO was normalized across the plate. Antagonists were added to the plate using a Tecan D300 Digital Dispenser at nanoliter/picoliter volumes and 2 μL of agonists in assay buffer were added to each well for a final assay volume of 14 μL. Plates were covered and incubated for 1 h at 37° C. and then for 1 h at room temperature. IP-1 standards and HTRF detection reagents were added according to an IP-One—Gq Kit purchased from Cisbio (part number 621PAPEJ) and incubated in the dark for 1 h at room temperature. Assay plates were read on either a Molecular Devices SpectraMax iD5 plate reader or a BMG ClarioStar plate reader. The HTRF ratio was calculated form the raw data and graphed using GraphPad Prism to calculate IC₅₀ values. Data are expressed as average IC₅₀ values and average percent antagonism calculated as a percent of the maximum efficacy response. For these studies, the variability of the data was assessed by determining the standard error of mean (SEM).

Activity data for selected MRGPRD antagonists (versus 100 μM β-Alanine agonist) are displayed in Table 26. The activity ranges are denoted as follows: “+++++” denotes antagonist activity<100 nM; “++++” denotes antagonist activity between 100 and 500 nM; “+++” denotes activity between 500 and 1000 nM; “++” denotes activity between 1000 and 3000 nM; and “+” denotes activity>3000 nM.

TABLE 26
Activity data for selected MRGPRD antagonists

| | mRGPRD Antagonist |
|---|---|
| Cpd No. | Activity |
| 1-4 | ++++ |
| 1-5 | +++ |
| 1-6 | +++ |
| 1-7 | +++ |
| 1-9 | ++ |
| 1-19 | +++ |
| 2-1 | + |
| 2-2 | +++ |
| 2-3 | +++ |
| 2-4 | +++ |
| 2-5 | ++ |
| 2-6 | ++ |
| 2-7 | ++ |
| 2-8 | ++ |
| 2-9 | ++ |
| 2-10 | ++ |
| 2-11 | ++ |
| 2-13 | + |
| 2-14 | + |
| 3-1 | +++ |
| 3-2 | ++ |
| 3-3 | ++ |
| 3-4 | ++ |
| 3-5 | +++ |
| 3-6 | ++ |
| 4-1 | + |
| 4-2 | ++++ |
| 4-3 | ++ |
| 4-4 | ++ |
| 4-5 | ++ |
| 4-6 | ++ |
| 4-7 | ++ |
| 4-8 | ++ |
| 4-9 | ++ |
| 4-10 | ++ |
| 4-11 | ++ |
| 4-12 | ++ |
| 4-13 | +++ |
| 5-1 | + |
| 5-2 | + |
| 5-3 | +++ |
| 5-4 | +++ |
| 5-5 | +++ |
| 5-6 | ++ |
| 5-7 | ++ |
| 6-1 | ++ |
| 6-2 | ++++ |
| 6-3 | +++ |
| 6-4 | ++ |
| 6-5 | ++ |
| 7-1 | + |
| 7-2 | + |
| 7-3 | + |
| 7-4 | + |
| 7-5 | + |
| 7-7 | + |
| 7-8 | + |
| 7-9 | + |
| 7-10 | + |
| 7-11 | +++++ |
| 7-12 | +++++ |
| 7-13 | +++++ |
| 7-14 | +++++ |
| 7-15 | +++++ |
| 7-16 | +++++ |
| 7-17 | +++++ |
| 7-18 | +++++ |
| 7-19 | +++++ |
| 7-20 | +++++ |
| 7-21 | ++++ |
| 7-22 | ++++ |
| 7-23 | ++++ |
| 7-24 | ++++ |
| 7-25 | ++++ |
| 7-26 | ++++ |
| 7-27 | ++++ |
| 7-28 | ++++ |
| 7-29 | ++++ |
| 7-30 | ++++ |
| 7-31 | ++++ |
| 7-32 | ++++ |
| 7-33 | ++++ |
| 7-34 | ++++ |
| 7-35 | ++++ |
| 7-36 | ++++ |
| 7-37 | ++++ |
| 7-38 | ++++ |
| 7-39 | ++++ |
| 7-40 | ++++ |
| 7-41 | ++++ |
| 7-42 | ++++ |
| 7-43 | ++++ |
| 7-44 | ++++ |
| 7-45 | ++++ |
| 7-46 | ++++ |
| 7-47 | ++++ |
| 7-48 | ++++ |
| 7-49 | ++++ |
| 7-50 | ++++ |
| 7-51 | ++++ |
| 7-52 | ++++ |
| 7-53 | ++++ |
| 7-54 | ++++ |
| 7-55 | ++++ |
| 7-56 | ++++ |
| 7-57 | ++++ |
| 7-58 | ++++ |
| 7-59 | ++++ |
| 7-60 | ++++ |
| 7-61 | ++++ |
| 7-62 | ++++ |
| 7-63 | ++++ |
| 7-64 | ++++ |
| 7-65 | ++++ |
| 7-66 | ++++ |
| 7-67 | ++++ |
| 7-68 | ++++ |
| 7-69 | ++++ |
| 7-70 | ++++ |
| 7-71 | ++++ |
| 7-72 | ++++ |
| 7-73 | ++++ |
| 7-74 | ++++ |
| 7-75 | ++++ |
| 7-76 | ++++ |
| 7-77 | ++++ |
| 7-78 | ++++ |
| 7-79 | ++++ |
| 7-80 | ++++ |
| 7-81 | ++++ |
| 7-82 | ++++ |
| 7-83 | ++++ |
| 7-84 | ++++ |
| 7-85 | ++++ |
| 7-86 | ++++ |
| 7-87 | ++++ |
| 7-88 | ++++ |
| 7-89 | ++++ |
| 7-90 | ++++ |
| 7-91 | ++++ |
| 7-92 | ++++ |
| 7-93 | ++++ |
| 7-94 | ++++ |
| 7-95 | ++++ |
| 7-96 | ++++ |
| 7-97 | ++++ |
| 7-98 | ++++ |
| 7-99 | ++++ |
| 7-100 | ++++ |
| 7-101 | ++++ |
| 7-102 | ++++ |
| 7-103 | ++++ |
| 7-104 | ++++ |
| 7-105 | ++++ |
| 7-106 | ++++ |
| 7-107 | ++++ |
| 7-108 | ++++ |
| 7-109 | ++++ |
| 7-110 | ++++ |
| 7-111 | ++++ |
| 7-112 | ++++ |
| 7-113 | +++ |
| 7-114 | +++ |
| 7-115 | +++ |
| 7-116 | +++ |
| 7-117 | +++ |
| 7-118 | +++ |
| 7-119 | +++ |
| 7-120 | +++ |
| 7-121 | +++ |
| 7-122 | +++ |
| 7-123 | +++ |
| 7-124 | +++ |
| 7-125 | +++ |
| 7-126 | +++ |
| 7-127 | +++ |
| 7-128 | +++ |
| 7-129 | +++ |
| 7-130 | +++ |
| 7-131 | +++ |
| 7-132 | +++ |
| 7-133 | +++ |
| 7-134 | +++ |
| 7-135 | +++ |
| 7-136 | +++ |
| 7-137 | +++ |
| 7-138 | +++ |
| 7-139 | +++ |
| 7-140 | +++ |
| 7-141 | +++ |
| 7-142 | +++ |
| 7-143 | +++ |
| 7-144 | +++ |
| 7-145 | +++ |
| 7-146 | +++ |
| 7-147 | +++ |
| 7-148 | +++ |
| 7-149 | +++ |
| 7-150 | +++ |
| 7-151 | +++ |
| 7-152 | +++ |
| 7-153 | +++ |
| 7-154 | +++ |
| 7-155 | +++ |
| 7-156 | +++ |
| 7-157 | +++ |
| 7-158 | +++ |
| 7-159 | +++ |
| 7-160 | +++ |
| 7-161 | +++ |
| 7-162 | ++ |
| 7-163 | ++ |
| 7-164 | ++ |
| 7-165 | ++ |
| 7-166 | ++ |
| 7-167 | ++ |
| 7-168 | ++ |
| 7-169 | ++ |
| 7-170 | ++ |
| 7-171 | ++ |
| 7-172 | ++ |
| 7-173 | ++ |
| 7-174 | ++ |
| 7-175 | ++ |
| 7-176 | ++ |
| 7-177 | ++ |
| 7-178 | ++ |
| 7-179 | ++ |
| 7-180 | ++ |
| 7-181 | ++ |
| 7-182 | ++ |
| 7-183 | ++ |
| 7-184 | ++ |
| 7-185 | ++ |
| 7-186 | ++ |
| 7-187 | ++ |
| 7-188 | ++ |
| 7-189 | ++ |
| 7-190 | ++ |
| 7-191 | ++ |
| 7-192 | ++ |
| 7-193 | ++ |
| 7-194 | ++ |
| 7-195 | ++ |
| 7-196 | ++ |
| 7-197 | ++ |
| 7-198 | ++ |
| 7-199 | ++ |
| 7-200 | ++ |
| 7-201 | ++ |
| 7-202 | ++ |
| 7-203 | ++ |
| 7-204 | ++ |
| 7-205 | ++ |
| 7-206 | ++ |
| 7-207 | ++ |
| 7-208 | ++ |
| 7-209 | ++ |
| 7-210 | ++ |
| 7-211 | ++ |
| 7-212 | ++ |
| 7-213 | + |
| 7-214 | + |
| 7-215 | + |
| 7-216 | + |
| 7-217 | + |
| 7-218 | + |
| 7-219 | +++++ |
| 7-220 | +++++ |
| 7-221 | +++++ |
| 7-222 | +++++ |
| 7-223 | +++++ |
| 7-224 | +++++ |
| 7-225 | +++++ |
| 7-226 | ++++ |
| 7-227 | ++++ |
| 7-228 | ++++ |
| 7-229 | ++++ |
| 7-230 | ++++ |
| 7-231 | ++++ |
| 7-232 | ++++ |
| 7-233 | ++++ |
| 7-234 | ++++ |
| 7-235 | ++++ |
| 7-236 | ++++ |
| 7-237 | ++++ |
| 7-238 | ++++ |
| 7-239 | ++++ |
| 7-240 | ++++ |
| 7-241 | +++ |
| 7-242 | +++ |
| 7-243 | +++ |
| 7-244 | +++ |
| 7-245 | +++ |
| 7-246 | +++ |
| 7-247 | +++ |
| 7-248 | +++ |
| 7-249 | +++ |
| 7-250 | +++ |
| 7-251 | +++ |
| 7-252 | +++ |
| 7-253 | +++ |
| 7-255 | +++ |
| 7-256 | +++ |
| 7-257 | +++ |
| 7-258 | +++ |
| 7-259 | +++ |
| 7-260 | +++ |
| 7-261 | +++ |
| 7-262 | +++ |
| 7-263 | +++ |
| 7-264 | +++ |
| 7-265 | +++ |
| 7-266 | +++ |
| 7-267 | +++ |
| 7-268 | +++ |
| 7-269 | +++ |
| 7-270 | +++ |
| 7-271 | +++ |
| 7-272 | +++ |
| 7-273 | +++ |
| 7-274 | +++ |
| 7-275 | +++ |
| 7-276 | +++ |
| 7-277 | +++ |
| 7-278 | +++ |
| 7-279 | +++ |
| 7-280 | +++ |
| 7-281 | +++ |
| 7-282 | +++ |
| 7-283 | +++ |
| 7-284 | +++ |
| 7-285 | ++ |
| 7-286 | ++ |
| 7-287 | ++ |
| 7-288 | ++ |
| 7-289 | ++ |
| 7-290 | ++ |
| 7-291 | ++ |
| 7-292 | ++ |
| 7-293 | ++ |
| 7-294 | ++ |
| 7-295 | ++ |
| 7-296 | ++ |
| 7-297 | ++ |
| 7-298 | ++ |
| 7-299 | ++ |
| 7-300 | ++ |
| 7-301 | ++ |
| 7-302 | ++ |
| 7-303 | ++ |
| 7-304 | ++ |
| 7-305 | ++ |
| 7-306 | ++ |
| 7-307 | ++ |
| 7-308 | ++ |
| 7-309 | ++ |
| 7-310 | ++ |
| 7-311 | ++ |
| 7-312 | ++ |
| 7-313 | ++ |
| 7-314 | ++ |
| 7-315 | ++ |
| 7-316 | ++ |
| 7-317 | ++ |
| 7-318 | ++ |
| 7-319 | ++ |
| 7-320 | ++ |
| 7-321 | ++ |
| 7-322 | ++ |
| 7-323 | ++ |
| 7-324 | ++ |
| 7-325 | ++ |
| 7-326 | ++ |
| 7-327 | ++ |
| 7-328 | + |
| 7-329 | + |
| 7-330 | + |
| 7-331 | + |
| 7-332 | + |
| 7-333 | + |
| 7-334 | + |
| 7-335 | + |
| 8-1 | +++++ |
| 8-2 | +++++ |
| 8-3 | ++++ |
| 8-4 | ++ |
| 9-1 | +++++ |
| 9-2 | +++++ |
| 9-3 | +++++ |
| 9-4 | +++++ |
| 9-5 | +++++ |
| 9-6 | +++++ |
| 9-7 | ++++ |
| 9-8 | ++++ |
| 9-9 | ++++ |
| 9-10 | ++++ |
| 9-11 | ++++ |
| 9-12 | ++++ |
| 9-13 | ++++ |
| 9-14 | ++++ |
| 9-15 | ++++ |
| 9-16 | ++++ |
| 9-17 | ++++ |
| 9-18 | ++++ |
| 9-19 | ++++ |
| 9-20 | ++++ |
| 9-21 | ++++ |
| 9-22 | ++++ |
| 9-23 | ++++ |
| 9-24 | ++++ |
| 9-25 | ++++ |
| 9-26 | +++ |
| 9-27 | +++ |
| 9-28 | +++ |
| 9-29 | +++ |
| 9-30 | ++ |
| 9-31 | ++ |
| 9-32 | ++ |
| 9-33 | ++ |
| 9-34 | ++ |
| 9-35 | ++++ |
| 9-36 | ++++ |
| 9-37 | +++ |
| 10-1 | +++++ |
| 10-2 | + |
| 10-3 | +++++ |
| 10-4 | ++++ |
| 10-5 | ++++ |
| 10-6 | ++++ |
| 10-7 | ++ |
| 10-8 | ++ |
| 10-9 | +++++ |
| 10-10 | +++++ |
| 10-11 | +++++ |
| 10-12 | ++++ |
| 11-1 | ++++ |
| 11-2 | ++++ |
| 12-1 | ++++ |
| 12-2 | ++++ |
| 12-3 | +++ |
| 12-4 | +++ |
| 12-5 | +++ |
| 12-6 | ++ |
| 12-7 | ++ |
| 12-8 | ++ |
| 12-9 | ++ |
| 13-1 | ++ |
| 14-1 | + |
| 14-2 | + |
| 14-3 | + |
| 14-4 | + |
| 14-5 | + |
| 14-6 | + |
| 14-7 | + |
| 14-8 | + |
| 14-9 | + |
| 14-10 | + |
| 14-11 | + |
| 14-12 | + |
| 14-13 | + |
| 14-14 | + |
| 14-15 | ++++ |
| 14-16 | ++++ |
| 14-17 | ++++ |
| 14-18 | ++++ |
| 14-19 | +++ |
| 14-20 | +++ |
| 14-21 | +++ |
| 14-22 | +++ |
| 14-23 | +++ |
| 14-24 | +++ |
| 14-25 | +++ |
| 14-26 | +++ |
| 14-27 | +++ |
| 14-28 | ++ |
| 14-29 | ++ |
| 14-30 | ++ |
| 14-31 | ++ |
| 14-32 | ++ |
| 14-33 | ++ |
| 14-34 | ++ |
| 14-36 | ++ |
| 14-37 | ++ |
| 14-38 | ++ |
| 14-39 | ++ |
| 14-40 | ++ |
| 14-41 | ++ |
| 14-42 | ++ |
| 14-43 | ++ |
| 14-44 | ++ |
| 14-45 | ++ |
| 14-46 | ++ |
| 14-47 | ++ |
| 14-48 | ++ |
| 14-49 | ++ |
| 14-50 | ++ |
| 14-51 | ++ |
| 14-52 | ++ |
| 14-53 | ++ |
| 14-54 | ++ |
| 14-55 | ++ |
| 14-56 | ++ |
| 14-57 | ++ |
| 14-58 | ++ |
| 14-59 | ++ |
| 14-60 | ++ |
| 14-61 | ++ |
| 14-62 | ++ |
| 14-63 | ++ |
| 14-64 | ++ |
| 14-65 | ++ |
| 14-66 | ++ |
| 14-67 | ++ |
| 14-68 | ++ |
| 14-69 | ++ |
| 14-70 | ++ |
| 14-71 | + |
| 14-72 | + |
| 14-73 | + |
| 14-74 | + |
| 14-75 | + |
| 14-76 | + |
| 14-77 | + |
| 14-78 | + |
| 14-79 | + |
| 14-80 | + |
| 14-81 | + |
| 14-82 | + |
| 14-83 | + |
| 14-84 | + |
| 14-85 | + |
| 14-86 | + |
| 14-87 | + |
| 14-88 | ++ |
| 15-1 | +++++ |
| 15-2 | ++++ |
| 15-3 | +++ |
| 15-4 | +++ |
| 15-5 | +++ |
| 15-6 | ++ |
| 15-7 | ++ |
| 15-8 | ++ |
| 15-9 | + |
| 15-10 | + |
| 15-11 | ++++ |
| 15-12 | ++++ |
| 15-13 | +++ |
| 15-14 | ++++ |
| 15-15 | ++ |
| 15-16 | ++ |
| 15-17 | ++ |
| 15-18 | ++ |
| 15-19 | ++++ |
| 15-20 | ++++ |
| 15-21 | +++ |
| 15-22 | +++++ |
| 15-23 | ++++ |
| 15-24 | ++++ |
| 16-1 | +++++ |
| 16-2 | ++++ |
| 16-3 | ++++ |
| 16-4 | ++++ |
| 16-5 | ++++ |
| 17-1 | ++++ |
| 17-2 | ++++ |
| 17-3 | ++++ |
| 17-4 | ++++ |
| 17-5 | ++++ |
| 17-6 | ++++ |
| 17-7 | ++ |
| 17-8 | ++ |
| 17-9 | ++++ |
| 17-10 | +++++ |
| 17-11 | +++++ |
| 17-12 | ++++ |
| 17-13 | ++++ |
| 17-14 | ++++ |
| 17-15 | ++++ |
| 17-16 | ++++ |
| 17-17 | ++++ |
| 17-18 | ++++ |
| 17-19 | ++++ |
| 17-20 | ++++ |
| 17-21 | ++++ |
| 17-22 | ++++ |
| 17-23 | ++++ |
| 17-24 | ++++ |
| 17-25 | ++++ |
| 17-26 | +++ |
| 17-27 | +++ |
| 17-28 | +++ |
| 17-29 | +++ |
| 17-30 | +++ |
| 17-31 | ++ |
| 17-32 | ++ |
| 17-33 | ++ |
| 17-34 | + |
| 18-1 | ++++ |
| 18-2 | ++++ |
| 18-3 | +++ |
| 18-4 | ++ |
| 18-5 | ++ |
| 18-6 | ++ |
| 18-7 | ++ |
| 18-8 | ++ |
| 18-9 | ++ |
| 18-10 | ++ |
| 18-11 | +++++ |
| 18-12 | ++ |
| 18-13 | ++++ |
| 18-14 | ++++ |
| 18-15 | +++ |
| 18-16 | +++ |
| 18-17 | ++ |
| 18-18 | ++ |
| 18-19 | ++ |
| 18-20 | ++++ |
| 18-21 | ++ |
| 18-22 | + |
| 18-23 | ++++ |
| 18-24 | +++ |
| 18-25 | +++ |
| 18-26 | ++ |
| 18-27 | ++ |
| 18-28 | ++ |
| 18-29 | + |
| 18-30 | + |
| 19-1 | +++ |
| 19-2 | ++++ |
| 19-3 | +++ |
| 19-4 | ++++ |
| 20-1 | +++++ |
| 21-1 | +++++ |
| 21-2 | ++++ |
| 22-1 | ++++ |
| 22-2 | +++ |
| 22-3 | ++ |
| 22-4 | ++ |
| 23-1 | ++++ |
| 24-1 | ++ |
| 25-1 | ++ |
| 25-2 | ++ |

All of the U.S. patents, U.S. patent application publications, U.S. patent applications, foreign patents, foreign patent applications and non-patent publications referred to in this specification and/or listed in the Application Data Sheet are incorporated herein by reference, in their entirety. In addition, the terms used in the following claims should not be construed as limited to the specific embodiments disclosed in the specification but should be construed to include all possible embodiments along with the full scope of equivalents to which such claims are entitled.

This application claims the benefit of priority to U.S. Provisional Application No. 63/367,012, filed Jun. 24, 2022, which application is hereby incorporated by reference in its entirety.

1. A compound having the following structure (I): 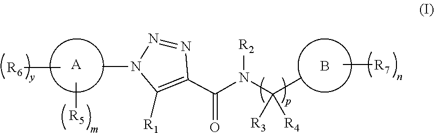
or a pharmaceutically acceptable salt, isomer, hydrate, solvate or isotope thereof, wherein:
when A is a monocyclic carbocyclic or monocyclic heterocyclic ring, then y is 1-4, or when A is a bicyclic carbocyclic or bicyclic heterocyclic ring, then y is 0-5;
B is a carbocyclic or heterocyclic ring;
R₁ is isopropyl, tert-butyl, cyclopropyl, cyclobutyl or cyclopentyl;
R₂ is H or alkyl, or R₂ joins to B to form a ring;
R₃ and R₄ are each independently H or alkyl, or R₃ and R₄ join together to form a carbocyclic ring;
R₅ is halo, OH, CN, alkyl, haloalkyl, alkoxy, haloalkoxy, alkoxyalkyl, alkoxyalkenyl, cyanoalkyl, alkylcarbonyl, aminylcarbonyl, cycloalkyl, heterocyclyl, aryl or heteroaryl;
R₆ is carbocyclyl or heterocyclyl, optionally substituted with one or more R₈;
R₇ is aminyl, halo, OH, CN, alkyl, alkenyl, alkynyl, haloalkyl, alkoxy, alkoxyalkyl, alkylcarbonyl, cycloalkyl, heterocyclyl, heteroaryl, alkylsulfonyl or alkylsulfonylaminyl;
R₈ is aminyl, halo, OH, CN, alkyl, alkenyl, alkynyl, haloalkyl, haloalkoxy, alkoxy, alkoxyalkyl, hydroxylalkyl, aminylalkyl, cyanoalkyl, carboxyl, alkylcarbonyl, aminylcarbonyl, cycloalkyl, heterocyclyl, aryl, arylalkyl, heteroaryl, heteroarylalkyl, alkylsulfonyl, aminylsulfonyl, cycloalkylalkyl, and wherein two R₈ may join together to form a carbocyclic or heterocyclic ring;
n is 0-4,
m is 0-4; and
p is 0-5.

2. The compound of claim 1, having the following structure (I′): 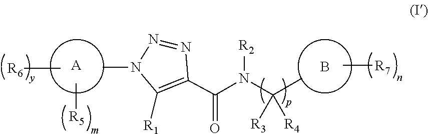
or a pharmaceutically acceptable salt, isomer, hydrate, solvate or isotope thereof, wherein:
when A is a monocyclic carbocyclic or monocyclic heterocyclic ring; and
y is 1-4.

3. The compound of claim 1 or 2, wherein A is a monocyclic carbocyclic ring.

4. The compound of claim 3, wherein A is a saturated or partially saturated monocyclic carbocyclic ring.

5. The compound of claim 3, wherein A is aryl.

6. The compound of claim 5, wherein A is phenyl.

7. The compound of claim 1 or 2, wherein A is a monocyclic heterocyclic ring.

8. The compound of claim 7, wherein A is a heteroaryl ring.

9. The compound of claim 1, having the following structure (I″): 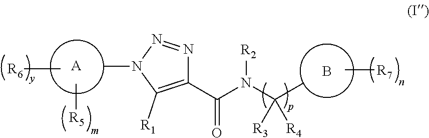
or a pharmaceutically acceptable salt, isomer, hydrate, solvate or isotope thereof, wherein:
when A is a bicyclic carbocyclic or bicyclic heterocyclic ring; and
y is 0-5.

10. The compound of claim 1 or 9, wherein A is a bicyclic carbocyclic ring.

11. The compound of claim 1 or 3, wherein A is a bicyclic heterocyclic ring.

12. The compound of claim 1 or 3, wherein A is a bicyclic heteroaryl ring.

13. The compound of claim 1, wherein A has one of the following structures:

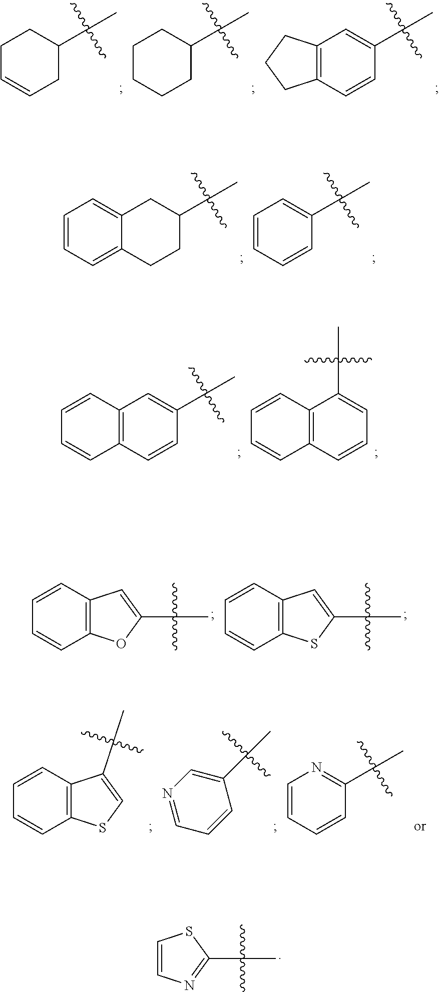

14. The compound of claim 1, having the following structure (II): 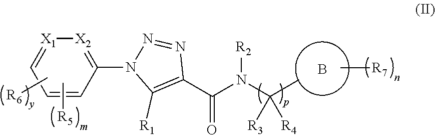
or a pharmaceutically acceptable salt, isomer, hydrate, solvate or isotope thereof, wherein:
X₁ and X₂ are both C, or X₁ is N and X₂ is C, or X₁ is C and X₂ is N;
and y is 1-4.

15. The compound of claim 14, having the following structure (II′): 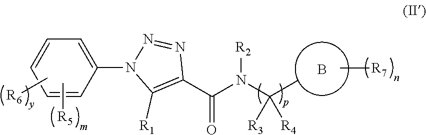
or a pharmaceutically acceptable salt, isomer, hydrate, solvate or isotope thereof, wherein:
y is 1-4.

16. The compound of claim 1, having the following structure (III): 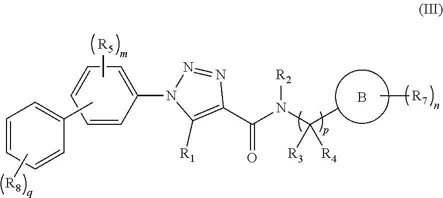
or a pharmaceutically acceptable salt, isomer, hydrate, solvate or isotope thereof, wherein:
q is 0-5.

17. The compound of claim 1, having the following structure (IV): 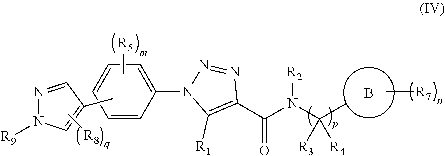
or a pharmaceutically acceptable salt, isomer, hydrate, solvate or isotope thereof, wherein:
R₉ is H, alkyl, haloalkyl, alkoxyalkyl, aminylsulfonyl, cycloalkyl, cycloalkylalkyl, heterocyclyl, heterocyclylalkyl, aryl, arylalkyl, heteroaryl, or heteroarylalkyl; and
q is 0-2.

18. The compound of claim 1 having the following structure (V): 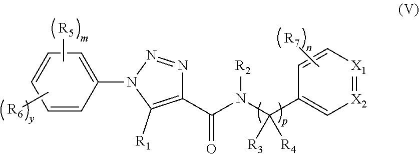
or a pharmaceutically acceptable salt, isomer, hydrate, solvate or isotope thereof, wherein:
one of X₁ and X₂ is N and the other is CH optionally substituted by R₇;
n is 0 to 4; and
y is 1-4.

19. The compound of claim 18, wherein X₁ is N, X₂ is CH optionally substituted by R₇, p is 0, and R₆ is a heterocyclic ring.

20. The compound of claim 18, wherein X₁ is N, X₂ is CH optionally substituted by R₇, p is 1, and R₆ is a heterocyclic ring.

21. The compound of claim 19 or 20, wherein R₆ is a 5-membered heterocyclic ring.

22. The compound of claim 19 or 20, wherein R₆ is a 6-membered heterocyclic ring.

23. The compound of claim 19 or 20, wherein R₆ is a 5-membered heteroaryl ring.

24. The compound of claim 19 or 20, wherein R₆ is a 6-membered heteroaryl ring.

25. The compound of claim 18, having the following structure (VA): 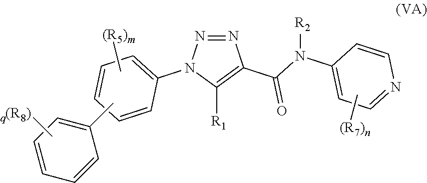
or a pharmaceutically acceptable salt, isomer, hydrate, solvate or isotope thereof, wherein:
n is 0-4; and
q is 0-5.

26. The compound of claim 18, having the following structure (VB): 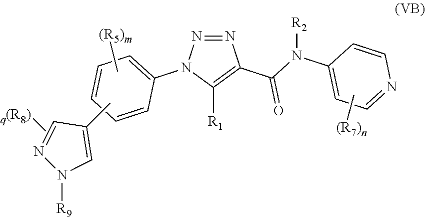
or a pharmaceutically acceptable salt, isomer, hydrate, solvate or isotope thereof, wherein:
R₉ is H, alkyl, haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl;
n is 0-4; and
q is 0-2.

27. The compound of claim 18, having the following structure (VC): 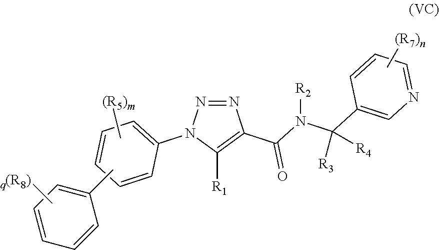
or a pharmaceutically acceptable salt, isomer, hydrate, solvate or isotope thereof, wherein:
n is 0-4; and
q is 0-5.

28. The compound of claim 18, having the following structure (VD): 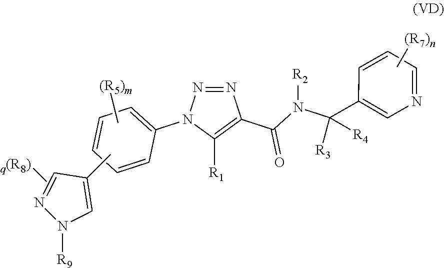
or a pharmaceutically acceptable salt, isomer, hydrate, solvate or isotope thereof, wherein:
R₉ is H, alkyl, haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl;
n is 0-4; and
q is 0-2.

29. The compound of claim 1, having the following structure (VI): 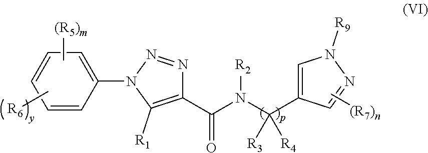
or a pharmaceutically acceptable salt, isomer, hydrate, solvate or isotope thereof, wherein:
R₉ is H, alkyl, haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl;
n is 0-2; and
y is 1-4.

30. The structure (VIA): 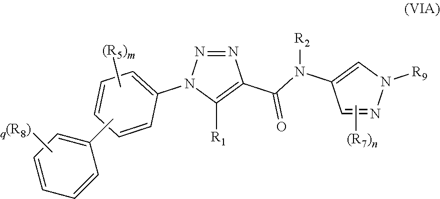
or a pharmaceutically acceptable salt, isomer, hydrate, solvate or isotope thereof, wherein:
R₉ is H, alkyl, haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl;
n is 0-4; and
q is 0-5.

31. The compound of claim 29, having the following structure (VIB): 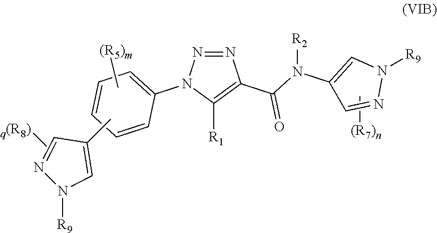
or a pharmaceutically acceptable salt, isomer, hydrate, solvate or isotope thereof, wherein:
each R₉ is independently H, alkyl, haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl;
n is 0-2, and
q is 0-2.

32. The compound of claim 29, having the following structure (VIC): 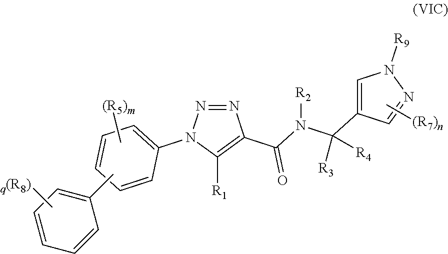
or a pharmaceutically acceptable salt, isomer, hydrate, solvate or isotope thereof, wherein:
R₉ is H, alkyl, haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl;
n is 0-2; and
q is 0-5.

33. The compound of claim 29, having the following structure (VID): 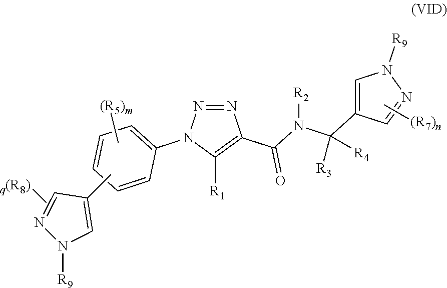
or a pharmaceutically acceptable salt, isomer, hydrate, solvate or isotope thereof, wherein:
each R₉ is independently H, alkyl, haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl;
n is 0-2; and
q is 0-2.

34. The compound of claim 1, having the following structure (VII): 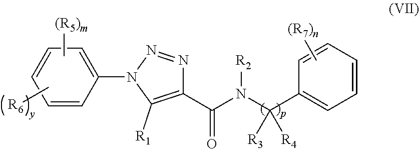
or a pharmaceutically acceptable salt, isomer, hydrate, solvate or isotope thereof, wherein:
y is 1-4.

35. The compound of claim 1, having the following structure (VIII): 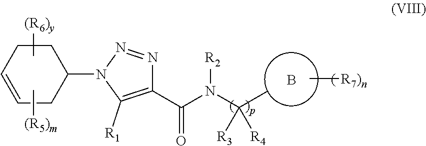
or a pharmaceutically acceptable salt, isomer, hydrate, solvate or isotope thereof, wherein:
y is 1-4.

36. The compound of any one of claims 1-17 or 35, wherein B is a carbocyclic ring.

37. The compound of claim 36, wherein B is a saturated carbocyclic ring.

38. The compound of claim 36, wherein B is an aryl ring.

39. The compound of any one of claims 1-17 or 35, wherein B is a heterocyclic ring.

40. The compound of claim 39, wherein B is a saturated heterocyclic ring.

41. The compound of claim 39, wherein B is a heteroaryl ring.

42. The compound of any one of claims 1-17 or 35, wherein B has one of the following structures: 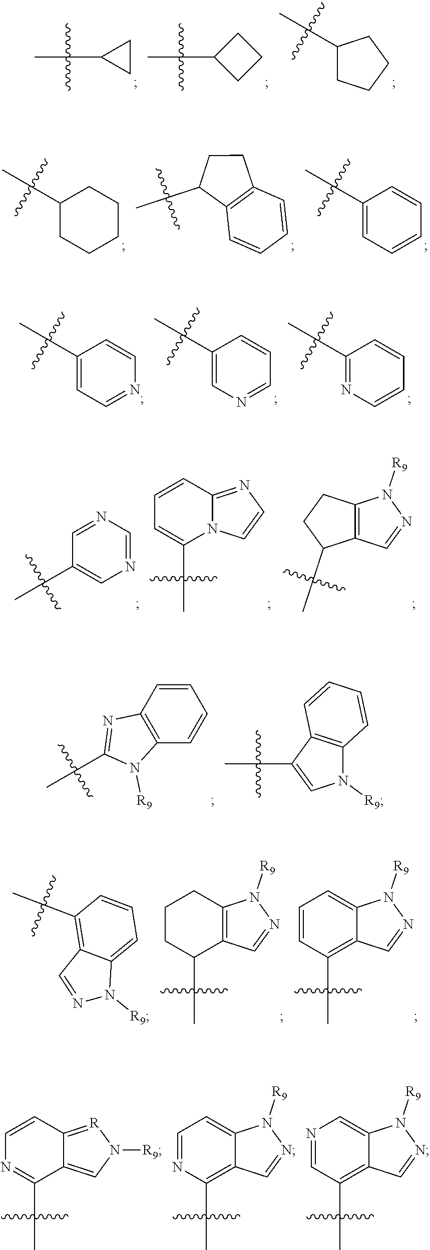 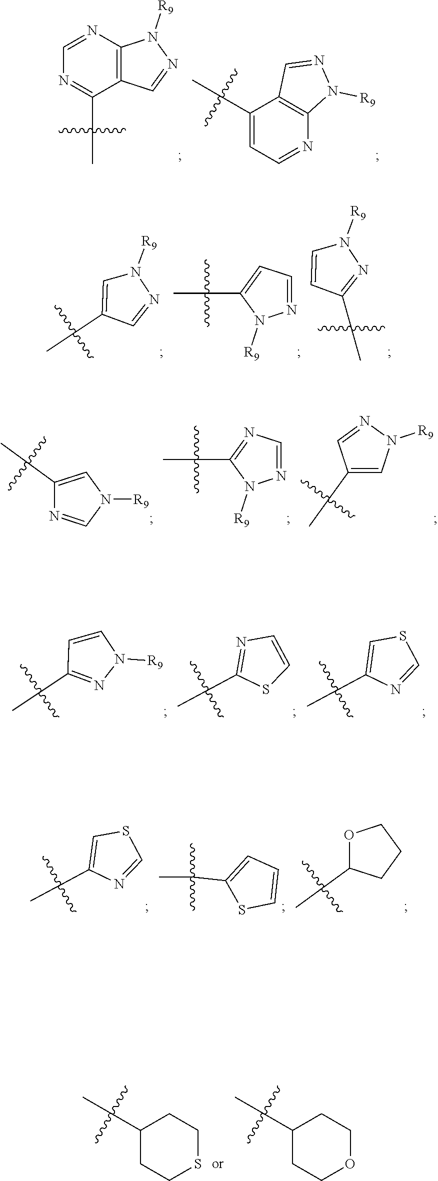
wherein B is optionally substituted by 0-4 R₇; and
wherein R₉ is H, alkyl, or haloalkyl, alkoxyalkyl, aminylsulfonyl, cycloalkyl, cycloalkylalkyl, heterocyclyl, heterocyclylalkyl, aryl, arylalkyl, heteroaryl, or heteroarylalkyl.

43. The compound of any one of claims 1-42, wherein R₁ is isopropyl.

44. The compound of any one of claims 1-42, wherein R₁ is tert-butyl.

45. The compound of any one of claims 1-42, wherein R₁ is cyclopropyl.

46. The compound of any one of claims 1-42, wherein R₁ is cyclobutyl.

47. The compound of any one of claims 1-42, wherein R₁ is cyclopentyl.

48. The compound of any one of claims 1-47, wherein R₂ is H.

49. The compound of any one of claims 1-47, wherein R₂ is alkyl.

50. The compound of claim 49, wherein R₂ is methyl.

51. The compound of any one of claims 1-47, wherein R₂ joins to B to form a ring.

52. The compound of claim 51, wherein R₂ joins to B to form a ring having the following structure: 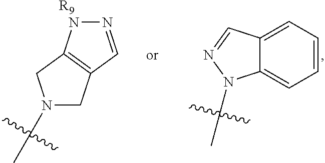
wherein the above structures are optionally substituted by 0-4 R₇; and
wherein R₉ is H, alkyl, or haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl.

53. The compound of any one of claims 1-24, 27-29, or 32-52, wherein R₃ and R₄ are both H.

54. The compound of any one of claims 1-24, 27-29, or 32-52, wherein R₃ and R₄ are both alkyl.

55. The compound of any one of claims 1-24, 27-29, or 32-52, wherein one of R₃ and R₄ is H and the other is alkyl.

56. The compound of claim 54 or 55, wherein the alkyl is methyl.

57. The compound of claim 54 or 55, wherein the alkyl is ethyl.

58. The compound of claim 54 or 55, wherein the alkyl is isopropyl.

59. The compound of any one of claims 1-24, 27-29, or 32-52, wherein R₃ and R₄ join together to form a carbocyclic ring.

60. The compound of claim 59, wherein R₃ and R₄ join together to form a 3- to 5-membered carbocyclic ring.

61. The compound of claim 60, wherein R₃ and R₄ join together to form cyclopropyl.

62. The compound of claim 60, wherein R₃ and R₄ join together to form cyclobutyl.

63. The compound of claim 60, wherein R₃ and R₄ join together to form cyclopentyl.

64. The compound of any one of claims 1-63, wherein R₅ is halo.

65. The compound of claim 64, wherein R₅ is F.

66. The compound of claim 64, wherein R₅ is Cl.

67. The compound of any one of claims 1-63, wherein R₅ is OH.

68. The compound of any one of claims 1-63, wherein R₅ is CN.

69. The compound of any one of claims 1-63, wherein R₅ is alkyl.

70. The compound of claim 69, wherein R₁ is methyl.

71. The compound of claim 69, wherein R₅ is ethyl.

72. The compound of any one of claims 1-63, wherein R₅ is haloalkyl.

73. The compound of claim 72, wherein R₅ is CF₃.

74. The compound of any one of claims 1-63, wherein R₅ is alkoxy.

75. The compound of claim 74, wherein R₅ is methoxy.

76. The compound of any one of claims 1-63, wherein R₅ is haloalkoxy.

77. The compound of claim 76, wherein R₅ is OCF₃.

78. The compound of any one of claims 1-63, wherein R₅ is alkoxyalkyl.

79. The compound of claim 78, wherein R₅ is CH₂OCH₃.

80. The compound of any one of claims 1-63, wherein R₅ is alkoxyalkenyl.

81. The compound of claim 80, wherein R₅ is

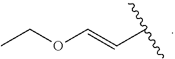 [SMILES: */C=C/OCC]

82. The compound of any one of claims 1-63, wherein R₅ is cyanoalkyl.

83. The compound of claim 82, wherein R₅ is CH₂CN.

84. The compound of any one of claims 1-63, wherein R₅ is alkylcarbonyl.

85. The compound of claim 84, wherein R₅ is C(O)CH₃.

86. The compound of any one of claims 1-63, wherein R₅ is aminylcarbonyl.

87. The compound of claim 86, wherein R₅ is C(O)NH₂.

88. The compound of any one of claims 1-63, wherein R₅ is cycloalkyl.

89. The compound of claim 88, wherein R₅ is C₃-C₅ cycloalkyl.

90. The compound of claim 89, wherein R₅ is cyclopropyl.

91. The compound of any one of claims 1-63, wherein R₅ is heterocyclyl.

92. The compound of any one of claims 1-63, wherein R₅ is aryl.

93. The compound of any one of claims 1-63, wherein R₅ is heteroaryl.

94. The compound of any one of claims 1-63, wherein R₅ has one of the following structures:

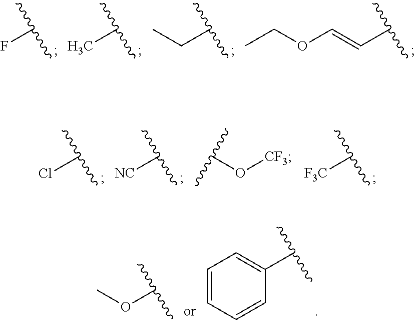

95. The compound of any one of claims 1-17, 18, 25, 27, 29-30, 32, or 34-94, wherein R₆ is carbocyclyl optionally substituted with one or more R₈.

96. The compound of any one of claims 1-15, 17-24, 26, 28-29, 31, or 33-94, wherein R₆ is heterocyclyl optionally substituted with one or more R₈.

97. The compound of any one of claims 1-94, wherein R₆ has one of the following structures: 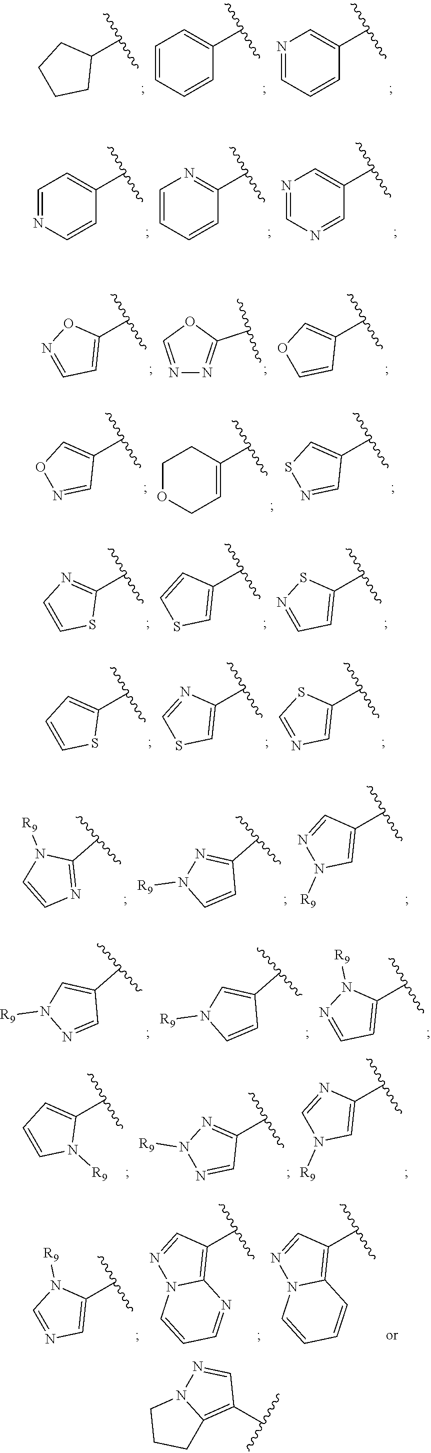
wherein R₆ is optionally substituted with one or more R₈; and
wherein R₉ is H, alkyl, or haloalkyl, aminylsulfonyl, alkoxyalkyl, cycloalkyl, cycloalkylalkyl, heterocyclyl, heterocyclylalkyl, aryl, arylalkyl, heteroaryl, or heteroarylalkyl.

98. The compound of any one of claims 1-97, wherein R₇ is aminyl.

99. The compound of any one of claims 1-97, wherein R₇ is halo.

100. The compound of claim 99, wherein R₇ is F.

101. The compound of claim 99, wherein R₇ is Cl.

102. The compound of any one of claims 1-97, wherein R₇ is OH.

103. The compound of any one of claims 1-97, wherein R₇ is CN.

104. The compound of any one of claims 1-97, wherein R₇ is alkyl.

105. The compound of claim 104, wherein R₇ is methyl.

106. The compound of claim 104, wherein R₇ is ethyl.

107. The compound of claim 104, wherein R₇ is n-propyl.

108. The compound of claim 104, wherein R₇ is isopropyl.

109. The compound of claim 104, wherein R₇ is tert-butyl.

110. The compound of any one of claims 1-97, wherein R₇ is alkenyl.

111. The compound of any one of claims 1-97, wherein R₇ is alkynyl.

112. The compound of claim 111, wherein R₇ is ethynyl.

113. The compound of any one of claims 1-97, wherein R₇ is haloalkyl.

114. The compound of claim 113, wherein R₇ is CF₃.

115. The compound of claim 113, wherein R₇ is CHF₂.

116. The compound of any one of claims 1-97, wherein R₇ is alkoxy.

117. The compound of claim 116, wherein R₇ is methoxy.

118. The compound of any one of claims 1-97, wherein R₇ is alkoxyalkyl.

119. The compound of claim 118, wherein R₇ is

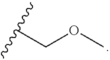 [SMILES: *COC]

120. The compound of any one of claims 1-97, wherein R₇ is alkylcarbonyl.

121. The compound of claim 120, wherein R₇ is C(O)CH₃.

122. The compound of any one of claims 1-97, wherein R₇ is cycloalkyl.

123. The compound of claim 122, wherein R₇ is cyclopropyl.

124. The compound of claim 122, wherein R₇ is cyclobutyl.

125. The compound of claim 122, wherein R₇ is cyclopentyl.

126. The compound of any one of claims 1-97, wherein R₇ is heterocyclyl.

127. The compound of claim 126, wherein R₇ is morpholino.

128. The compound of any one of claims 1-97, wherein R₇ is heteroaryl.

129. The compound of claim 128, wherein R₇ is triazolyl.

130. The compound of any one of claims 1-97, wherein R₇ is alkylsulfonyl.

131. The compound of claim 130, wherein R₇ is

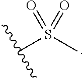 [SMILES: *S(C)(=O)=O]

132. The compound of any one of claims 1-97, wherein R₇ is alkylsulfonylaminyl.

133. The compound of claim 132, wherein R₇ is

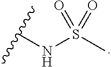 [SMILES: *NS(C)(=O)=O]

134. The compound of any one of claims 1-97, wherein R₇ has one of the following structures:

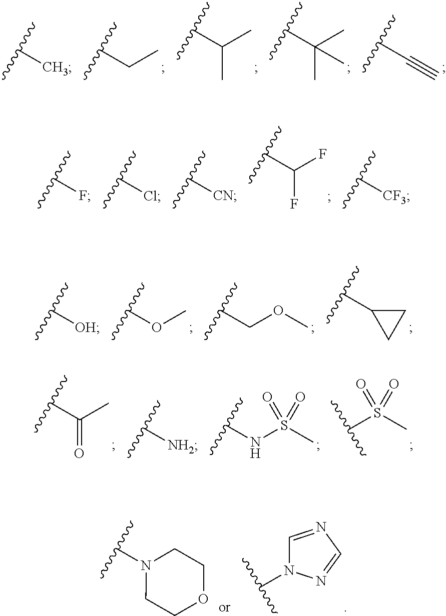

135. The compound of any one of claims 1-134, wherein R₈ is aminyl.

136. The compound of claim 135, wherein R₈ is

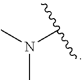 [SMILES: *N(C)C]

137. The compound of any one of claims 1-134, wherein R₈ is halo.

138. The compound of claim 134, wherein R₈ is F.

139. The compound of claim 134, wherein R₈ is Cl.

140. The compound of any one of claims 1-134, wherein R₈ is OH.

141. The compound of any one of claims 1-134, wherein R₈ is CN.

142. The compound of any one of claims 1-134, wherein R₈ is alkyl.

143. The compound of claim 142, wherein R₈ is methyl.

144. The compound of claim 142, wherein R₈ is ethyl.

145. The compound of claim 142, wherein R₈ is n-propyl.

146. The compound of claim 142, wherein R₈ is isopropyl.

147. The compound of claim 142, wherein R₈ is tert-butyl.

148. The compound of claim 142, wherein R₈ is

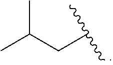 [SMILES: *CC(C)C]

149. The compound of any one of claims 1-134, wherein R₈ is alkenyl.

150. The compound of any one of claims 1-134, wherein R₈ is alkynyl.

151. The compound of claim 150, wherein R₈ is ethynyl.

152. The compound of any one of claims 1-134, wherein R₈ is haloalkyl.

153. The compound of claim 152, wherein R₈ is CF₃.

154. The compound of claim 152, wherein R₈ is CHF₂.

155. The compound of claim 152, wherein R₈ is CH₂CF₃.

156. The compound of any one of claims 1-134, wherein R₈ is haloalkoxy.

157. The compound of claim 156, wherein R₈ is OCF₃.

158. The compound of any one of claims 1-134, wherein R₈ is alkoxy.

159. The compound of claim 158, wherein R₈ is OCH₃.

160. The compound of claim 158, wherein R₈ is

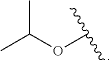 [SMILES: *OC(C)C]

161. The compound of any one of claims 1-134, wherein R₈ is alkoxyalkyl.

162. The compound of claim 161, wherein R₈ is

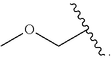 [SMILES: *COC]

163. The compound of any one of claims 1-134, wherein R₈ is hydroxylalkyl.

164. The compound of claim 163, wherein R₈ is

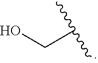 [SMILES: *CO]

165. The compound of claim 163, wherein R₈ is

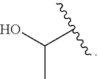 [SMILES: *C(C)O]

166. The compound of any one of claims 1-134, wherein R₈ is aminylalkyl.

167. The compound of claim 166, wherein R₈ is

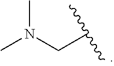 [SMILES: *CN(C)C]

168. The compound of any one of claims 1-134, wherein R₈ is cyanoalkyl.

169. The compound of claim 168, wherein R₈ is

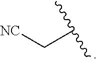 [SMILES: *CC#N]

170. The compound of any one of claims 1-134, wherein R₈ is carboxyl.

171. The compound of any one of claims 1-134, wherein R₈ is alkylcarbonyl.

172. The compound of claim 171, wherein R is

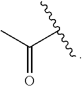 [SMILES: *C(C)=O]

173. The compound of claim 171, wherein R₈ is

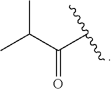 [SMILES: *C(=O)C(C)C]

174. The compound of any one of claims 1-134, wherein R₈ is aminylcarbonyl.

175. The compound of claim 174, wherein R₈ is

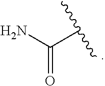 [SMILES: *C(N)=O]

176. The compound of claim 174, wherein R₈ is

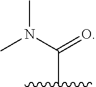 [SMILES: *C(=O)N(C)C]

177. The compound of any one of claims 1-134, wherein R₈ is cycloalkyl.

178. The compound of claim 177, wherein R₈ is cyclopropyl.

179. The compound of claim 177, wherein R₈ is cyclobutyl.

180. The compound of claim 177, wherein R₈ is cyclopentyl.

181. The compound of any one of claims 1-134, wherein R₈ is heterocyclyl.

182. The compound of claim 181, wherein R is tetrahydropyranyl.

183. The compound of any one of claims 1-134, wherein R₈ is aryl.

184. The compound of claim 183, wherein R₈ is phenyl.

185. The compound of any one of claims 1-134, wherein R₈ is arylalkyl.

186. The compound of claim 185, wherein R₈ is

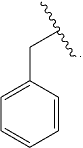 [SMILES: *Cc1ccccc1]

187. The compound of any one of claims 1-134, wherein R₈ is heteroaryl.

188. The compound of any one of claims 1-134, wherein R₈ is heteroarylalkyl.

189. The compound of claim 188, wherein R₈ is

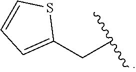 [SMILES: *Cc1cccs1]

190. The compound of any one of claims 1-134, wherein R₈ is alkylsulfonyl.

191. The compound of claim 190, wherein R₈ is

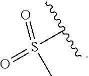 [SMILES: *S(C)(=O)=O]

192. The compound of any one of claims 1-134, wherein R₈ is aminylsulfonyl.

193. The compound of claim 192, wherein R₈ is

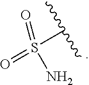 [SMILES: *S(N)(=O)=O]

194. The compound of claim 192, wherein R₈ is

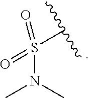 [SMILES: *S(=O)(=O)N(C)C]

195. The compound of any one of claims 1-134, wherein R₈ is cycloalkylalkyl.

196. The compound of claim 195, wherein R₈ is

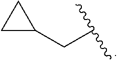 [SMILES: *CC1CC1]

197. The compound of any one of claims 1-134, wherein two R₈ join together to form a carbocyclic ring.

198. The compound of any one of claims 1-134, wherein two R₈ join together to form a heterocyclic ring.

199. The compound of any one of claims 1-134, wherein R₈ has one of the following structures:

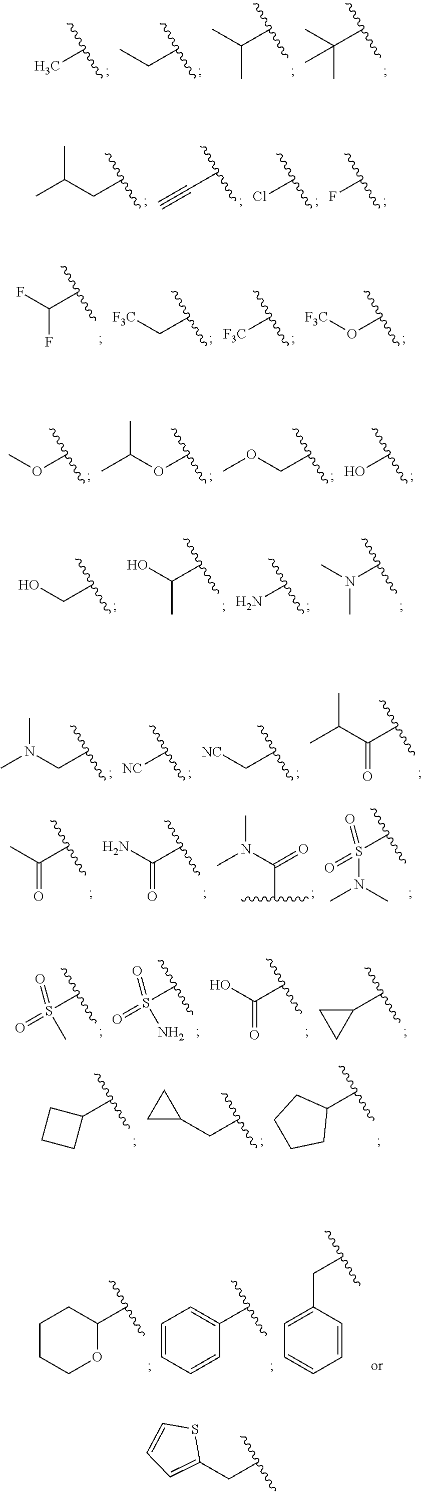

200. The compound of any one of claims 17, 26, 28-33, 36-199, wherein R₉ is H.

201. The compound of any one of claims 17, 26, 28-33, 36-199 wherein R₉ is alkyl.

202. The compound of claim 201, wherein R₉ is methyl.

203. The compound of claim 201, wherein R₉ is ethyl.

204. The compound of claim 201, wherein R₉ is n-propyl.

205. The compound of claim 201, wherein R₉ is isopropyl.

206. The compound of claim 201, wherein R₉ is tert-butyl.

207. The compound of claim 201, wherein R₉ is

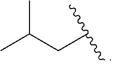 [SMILES: *CC(C)C]

208. The compound of any one of claims 17, 26, 28-33, 36-199, wherein R₉ is haloalkyl.

209. The compound of claim 208, wherein R₉ is CF₃.

210. The compound of claim 208, wherein R₉ is CHF₂.

211. The compound of claim 208, wherein R₉ is

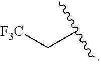 [SMILES: *CC(F)(F)F]

212. The compound of any one of claims 17, 26, 28-33, 36-199, wherein R₉ is alkoxyalkyl.

213. The compound of claim 212, wherein R₉ is

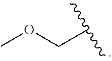 [SMILES: *COC]

214. The compound of any one of claims 17, 26, 28-33, 36-199, wherein R₉ is aminylsulfonyl.

215. The compound of claim 214, wherein R₉ is

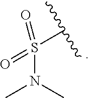 [SMILES: *S(=O)(=O)N(C)C]

216. The compound of any one of claims 17, 26, 28-33, 36-199, wherein R₉ is cycloalkyl.

217. The compound of claim 216, wherein R₉ is cyclopropyl.

218. The compound of claim 216, wherein R₉ is cyclobutyl.

219. The compound of claim 216, wherein R₉ is cyclopentyl.

220. The compound of any one of claims 17, 26, 28-33, 36-199, wherein R₉ is cycloalkylalkyl.

221. The compound of claim 220, wherein R₉ is

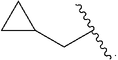 [SMILES: *CC1CC1]

222. The compound of any one of claims 17, 26, 28-33, 36-199, wherein R₉ is heterocyclyl.

223. The compound of claim 222, wherein R₉ is tetrahydropyranyl.

224. The compound of any one of claims 17, 26, 28-33, 36-199, wherein R₉ is heterocyclylalkyl.

225. The compound of any one of claims 17, 26, 28-33, 36-199, wherein R₉ is aryl.

226. The compound of claim 225, wherein R₉ is phenyl.

227. The compound of any one of claims 17, 26, 28-33, 36-199, wherein R₉ is arylalkyl.

228. The compound of claim 227, wherein R₉ is

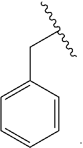 [SMILES: *Cc1ccccc1]

229. The compound of any one of claims 17, 26, 28-33, 36-199, wherein R₉ is heteroaryl.

230. The compound of any one of claims 17, 26, 28-33, 36-199, wherein R₉ is heteroarylalkyl.

231. The compound of claim 230, wherein R₉ is

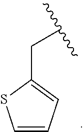 [SMILES: *Cc1cccs1]

232. The compound of any one of claims 17, 26, 28-33, 36-199, wherein R₉ has one of the following structures:

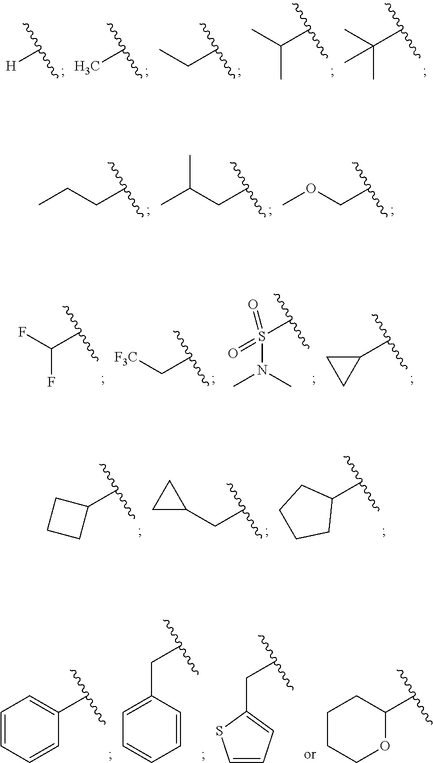

233. The compound of any one of claims 1-232, wherein n is 0.

234. The compound of any one of claims 1-232, wherein n is 1.

235. The compound of any one of claims 1-232, wherein n is 2.

236. The compound of any one of claims 1-232, wherein n is 3.

237. The compound of any one of claims 1-232, wherein n is 4.

238. The compound of any one of claims 1-237, wherein m is 0.

239. The compound of any one of claims 1-237, wherein m is 1.

240. The compound of any one of claims 1-237, wherein m is 2.

241. The compound of any one of claims 1-237, wherein m is 3.

242. The compound of any one of claims 1-237, wherein m is 4.

243. The compound of any one of claims 1-242, wherein p is 0.

244. The compound of any one of claims 1-242, wherein p is 1.

245. The compound of any one of claims 1-242, wherein p is 2.

246. The compound of any one of claims 1-242, wherein p is 3.

247. The compound of any one of claims 1-242, wherein p is 4.

248. The compound of any one of claims 1-242, wherein p is 5.

249. The compound of any one of claims 1-248, wherein q is 0.

250. The compound of any one of claims 1-248, wherein q is 1.

251. The compound of any one of claims 1-248, wherein q is 2.

252. A compound having the following structure (IX): 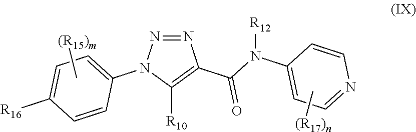
or a pharmaceutically acceptable salt, isomer, hydrate, solvate or isotope thereof, wherein:
R₁₀ is isopropyl, tert-butyl, cyclopropyl, cyclobutyl or cyclopentyl;
R₁₂ is H or alkyl;
R₁₅ is halo, alkyl, CN, OH, alkoxy, haloalkyl, cyanoalkyl or alkylcarbonyl;
R₁₆ is H, alkyl, CN, OH, alkoxy, haloalkyl, cyanoalkyl or alkylcarbonyl;
R₁₇ is halo, alkyl or haloalkyl;
m is 0-4;
n is 0-4;
provided that when R₁₆ is methyl and R₁ is cyclopropyl, then m or n is not 0; and
provided that when R₁₆ is H, R₁ is cyclopropyl, and m is 1, then R₁₅ is not F.

253. A compound having the following structure (IX): 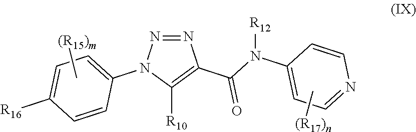
or a pharmaceutically acceptable salt, isomer, hydrate, solvate or isotope thereof, wherein:
(A) R₁₆ is H;
R₁₀ is isopropyl, tert-butyl, cyclopropyl, cyclobutyl or cyclopentyl;
R₁₂ is H or alkyl;
R₁₅ is Cl, Br, I, alkyl, CN, OH, alkoxy, haloalkyl, cyanoalkyl or alkylcarbonyl;
R₁₇ is halo, alkyl or haloalkyl;
m is 0-4; and
n is 0-4;
(B) R₁₆ is methyl and R₁₀ is cyclopropyl;
R₁₂ is H or alkyl;
R₁₅ is halo, alkyl, CN, OH, alkoxy, haloalkyl, cyanoalkyl or alkylcarbonyl;
R₁₇ is halo, alkyl or haloalkyl; and
m is 1-4 and n is 0-4, or m is 0-4 and n is 1-4;
(C) R₁₆ is methyl and R₁₀ is tert-butyl, cyclopropyl, cyclobutyl or cyclopentyl;
R₁₂ is H or alkyl;
R₁₅ is halo, alkyl, CN, OH, alkoxy, haloalkyl, cyanoalkyl or alkylcarbonyl;
R₁₇ is halo, alkyl or haloalkyl;
m is 0-4; and
n is 0-4; or
(D) R₁₆ is C₂-C₆ alkyl, CN, OH, alkoxy, haloalkyl, cyanoalkyl or alkylcarbonyl;
R₁₀ is isopropyl, tert-butyl, cyclopropyl, cyclobutyl or cyclopentyl;
R₁₂ is H or alkyl;
R₁₅ is halo, alkyl, CN, OH, alkoxy, haloalkyl, cyanoalkyl or alkylcarbonyl;
R₁₇ is halo, alkyl or haloalkyl;
m is 0-4; and
n is 0-4.

254. The compound of claim 252 or 253, wherein R₁₀ is isopropyl.

255. The compound of claim 252 or 253, wherein R₁₀ is tert-butyl.

256. The compound of claim 252 or 253, wherein R₁₀ is cyclopropyl.

257. The compound of claim 252 or 253, wherein R₁₀ is cyclobutyl.

258. The compound of claim 252 or 253, wherein R₁₀ is cyclopentyl.

259. The compound of any one of claims 252-258, wherein R₁₂ is H.

260. The compound of any one of claims 252-258, wherein R₁₂ is alkyl.

261. The compound of any one of claims 252-260, wherein R₁₅ is halo.

262. The compound of claim 261, wherein R₁₅ is F.

263. The compound of claim 261, wherein R₁₅ is Cl.

264. The compound of any one of claims 252-260, wherein R₁₅ is alkyl.

265. The compound of claim 264, wherein R₁₅ is methyl.

266. The compound of claim 264, wherein R₁₅ is ethyl.

267. The compound of any one of claims 252-260, wherein R₁₅ is CN.

268. The compound of any one of claims 252-260, wherein R₁₅ is OH.

269. The compound of any one of claims 252-260, wherein R₁₅ is alkoxy.

270. The compound of claim 269, wherein R₁₅ is methoxy.

271. The compound of any one of claims 252-260, wherein R₁₅ is haloalkyl.

272. The compound of claim 271, wherein R₁₅ is CF₃.

273. The compound of any one of claims 252-260, wherein R₁₅ is cyanoalkyl.

274. The compound of claim 273, wherein R₁₅ is CH₂CN.

275. The compound of any one of claims 252-260, wherein R₁₅ is alkylcarbonyl.

276. The compound of claim 275, wherein R₁₅ is C(O)CH₃.

277. The compound of any one of claims 252-276, wherein R₁₆ is H.

278. The compound of any one of claims 252-276, wherein R₁₆ is alkyl.

279. The compound of claim 278, wherein R₁₆ is methyl.

280. The compound of claim 278, wherein R₁₆ is ethyl.

281. The compound of any one of claims 252-276, wherein R₁₆ is CN.

282. The compound of any one of claims 252-276, wherein R₁₆ is OH.

283. The compound of any one of claims 252-276, wherein R₁₆ is alkoxy.

284. The compound of claim 283, wherein R₁₆ is methoxy.

285. The compound of any one of claims 252-276, wherein R₁₆ is haloalkyl.

286. The compound of claim 285, wherein R₁₆ is CF₃.

287. The compound of any one of claims 252-276, wherein R₁₆ is cyanoalkyl.

288. The compound of claim 287, wherein R₁₆ is CH₂CN.

289. The compound of claim any one of claims 252-276, wherein R₁₆ is alkylcarbonyl.

290. The compound of claim 289, wherein R₁₆ is C(O)CH₃.

291. The compound of any one of claims 252-290, wherein R₁₇ is halo.

292. The compound of claim 291, wherein R₁₇ is F.

293. The compound of claim 291, wherein R₁₇ is Cl.

294. The compound of any one of claims 252-290, wherein R₁₇ is alkyl.

295. The compound of claim 294, wherein R₁₇ is methyl.

296. The compound of any one of claims 252-290, wherein R₁₇ is haloalkyl.

297. The compound of claim 296, wherein R₁₇ is CF₃.

298. The compound of any one of claims 252-297, wherein m is 0.

299. The compound of any one of claims 252-297, wherein m is 1.

300. The compound of any one of claims 252-299, wherein n is 0.

301. The compound of any one of claims 252-299, wherein n is 1.

302. The compound of any one of claims 252-299, wherein n is 2.

303. A compound having the following structure (X) 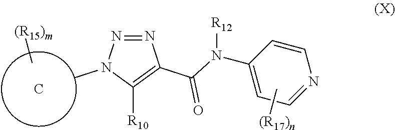
or a pharmaceutically acceptable salt, isomer, hydrate, solvate or isotope thereof, wherein:
C is a saturated carbocyclic ring;
R₁₀ is alkyl, cyclopropyl, cyclobutyl or cyclopentyl;
R₁₂ is H or alkyl;
R₁₅ is halo, alkyl, CN, OH, alkoxy, haloalkyl, cyanoalkyl or alkylcarbonyl;
R₁₇ is halo, alkyl or haloalkyl;
m is 0-4; and
n is 0-4.

304. The compound of claim 303, wherein C is cycloalkyl.

305. The compound of claim 304, wherein C is cyclopentyl.

306. The compound of claim 304, wherein C is cyclohexyl.

307. The compound of any one of claims 303-306, wherein R₁₀ is alkyl.

308. The compound of any one of claims 303-306, wherein R₁₀ is cyclopropyl.

309. The compound of any one of claims 303-306, wherein R₁₀ is cyclobutyl.

310. The compound of any one of claims 303-306, wherein R₁₀ is cyclopentyl.

311. The compound of any one of claims 303-310, wherein R₁₂ is H.

312. The compound of any one of claims 303-310, wherein R₁₂ is alkyl.

313. The compound of any one of claims 303-312, wherein R₁₂ is halo.

314. The compound of claim 313, wherein R₁₅ is F.

315. The compound of claim 313, wherein R₁₅ is Cl.

316. The compound of any one of claims 303-312, wherein R₁₅ is alkyl.

317. The compound of claim 316, wherein R₁₅ is methyl.

318. The compound of claim 316, wherein R₁₅ is ethyl.

319. The compound of any one of claims 303-312, wherein R₁₅ is CN.

320. The compound of any one of claims 303-312, wherein R₁₅ is OH.

321. The compound of any one of claims 303-312, wherein R₁₅ is alkoxy.

322. The compound of claim 321, wherein R₁₅ is methoxy.

323. The compound of any one of claims 303-312, wherein R₁₅ is haloalkyl.

324. The compound of claim 323, wherein R₁₅ is CF₃.

325. The compound of any one of claims 303-312, wherein R₁₅ is cyanoalkyl.

326. The compound of claim 325, wherein R₁₅ is CH₂CN.

327. The compound of any one of claims 303-312, wherein R₁₅ is alkylcarbonyl.

328. The compound of claim 327, wherein R₁₅ is C(O)CH₃.

329. The compound of any one of claims 303-328, wherein R₁₇ is halo.

330. The compound of claim 329, wherein R₁₇ is F.

331. The compound of claim 329, wherein R₁₇ is Cl.

332. The compound of any one of claims 303-328, wherein R₁₇ is alkyl.

333. The compound of claim 332, wherein R₁₇ is methyl.

334. The compound of any one of claims 303-328, wherein R₁₇ is haloalkyl.

335. The compound of claim 334, wherein R₁₇ is CF₃.

336. The compound of any one of claims 303-335, wherein m is 0.

337. The compound of any one of claims 303-335, wherein m is 1.

338. The compound of any one of claims 303-337, wherein n is 0.

339. The compound of any one of claims 303-337, wherein n is 1.

340. The compound of any one of claims 303-337, wherein n is 2.

341. A compound having the following structure (XI): 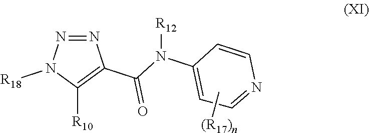
or a pharmaceutically acceptable salt, isomer, hydrate, solvate or isotope thereof, wherein:
R₁₀ is isopropyl, tert-butyl, cyclopropyl, cyclobutyl or cyclopentyl;
R₁₂ is H or alkyl;
R₁₇ is halo, alkyl or haloalkyl;
R₁₈ is an alkyl group optionally substituted with one or more R₁₉;
R₁₉ is halo, CN, OH, alkoxy, haloalkyl, or alkylcarbonyl; and
n is 0-4.

342. The compound of claim 341, wherein R₁₀ is isopropyl.

343. The compound of claim 341, wherein R₁₀ is tert-butyl.

344. The compound of claim 341, wherein R₁₀ is cyclopropyl.

345. The compound of claim 341, wherein R₁₀ is cyclobutyl.

346. The compound of claim 341, wherein R₁₀ is cyclopentyl.

347. The compound of any one of claims 341-346, wherein R₁₂ is H.

348. The compound of any one of claims 341-346, wherein R₁₂ is alkyl.

349. The compound of any one of claims 341-346, wherein R₁₇ is halo.

350. The compound of claim 349, wherein R₁₇ is F.

351. The compound of claim 349, wherein R₁₇ is Cl.

352. The compound of any one of claims 341-346, wherein R₁₇ is alkyl.

353. The compound of claim 352, wherein R₁₇ is methyl.

354. The compound of any one of claims 341-346, wherein R₁₇ is haloalkyl.

355. The compound of claim 354, wherein R₁₇ is CF₃.

356. The compound of any one of claims 341-355, wherein R₁₇ is C₁-C₄ alkyl optionally substituted with one R₁₉.

357. The compound of claim 356, wherein R₁₈ is methyl substituted with one R₁₉.

358. The compound of claim 356, wherein R₁₉ is ethyl substituted with one R₁₉.

359. The compound of any one of claims 341-358, wherein R₁₉ is halo.

360. The compound of claim 359, wherein R₁₉ is F.

361. The compound of claim 359, wherein R₁₉ is Cl.

362. The compound of any one of claims 341-358, wherein R₁₉ is CN.

363. The compound of any one of claims 341-358, wherein R₁₉ is OH.

364. The compound of any one of claims 341-358, wherein R₁₉ is haloalkyl.

365. The compound of claim 364, wherein R₁₉ is CF₃.

366. The compound of any one of claims 341-358, wherein R₁₉ is alkoxy.

367. The compound of claim 366, wherein R₁₉ is methoxy.

368. The compound of any one of claims 341-358, wherein R₁₉ is alkylcarbonyl.

369. The compound of claim 368, wherein R₁₉ is C(O)CH₃.

370. The compound of any one of claims 341-369, wherein n is 0.

371. The compound of any one of claims 341-369, wherein n is 1.

372. The compound of any one of claims 341-369, wherein n is 2.

373. A compound or a pharmaceutically acceptable salt, isomer, hydrate, solvate or isotope thereof, wherein the compound has one of the structures found in Table A.

374. A pharmaceutical composition comprising the compound of any one of claims 1-373, or a pharmaceutically acceptable salt, isomer, hydrate, solvate or isotope thereof and at least one pharmaceutically acceptable excipient.

375. A method of modulating a Mas-Related G-Protein Receptor (MRGPR) D by contacting MRGPRD with an effective amount of the compound of any one of claims 1-373 or the pharmaceutical composition of claim 374.

376. A method of treating a MRGPRD dependent condition by administering to a subject in need thereof an effective amount of the compound of any one of claims 1-373 or the pharmaceutical composition of claim 374.

377. The method of claim 376, wherein the MRGPRD dependent condition is a pain associated condition, an itch associated condition, an ocular associated condition, a cardiovascular and renal disease associate condition, an inflammatory or autoimmune disorder, or a cognitive impairment associated condition, or a cancer related condition.

378. The method of claim 377, wherein the pain associated condition is Acute Pain, Advanced Prostate Cancer, AIDS-Related Pain, Ankylosing Spondylitis, Arachnoiditis, Arthritis, Arthrofibrosis, Ataxic Cerebral Palsy, Autoimmune Atrophic Gastritis, Avascular Necrosis, Back Pain, Behcet's Disease (Syndrome), Burning Mouth Syndrome, Bursitis, Cancer Pain, Carpal Tunnel, Cauda Equina Syndrome, Central Pain Syndrome, Cerebral Palsy, Cervical Stenosis, Charcot-Marie-Tooth (CMT) Disease, Chronic Fatigue Syndrome (CFS), Chronic Functional Abdominal Pain (CFAP), Chronic Pain, Chronic Pancreatitis, Chronic Pelvic Pain Syndrome, Collapsed Lung (Pneumothorax), Complex Regional Pain Syndrome (RSD), Constipation, Corneal Neuropathic Pain, Crohn's Disease, Degenerative Disc Disease, Dental Pain, Dercum's Disease, Dermatomyositis, Diabetic Peripheral Neuropathy (DPN), Dystonia, Ehlers-Danlos Syndrome (EDS), Endometriosis, Eosinophilia-Myalgia Syndrome (EMS), Erythromelalgia, Fibromyalgia, Gout, Headaches, Herniated disc, Hydrocephalus, Inflammatory Bowel Disease, Intercostal Neuraligia, Interstitial Cystitis, Irritable Bowel syndrome (IBS), Juvenile Dermatositis (Dermatomyositis), Knee Injury, Leg Pain, Loin Pain-Haematuria Syndrome, Lupus, Lyme Disease, Medullary Sponge Kidney (MSK), Meralgia Paresthetica, Mesothelioma, Migraine, Musculoskeletal pain, Myofascial Pain, Myositis, Neck Pain, Neuropathic Pain, Occipital Neuralgia, Osteoarthritis, Paget's Disease, Parsonage Turner Syndrome, Pelvic Pain, Periodontitis Pain, Peripheral Neuropathy, Phantom Limb Pain, Pinched Nerve, Polycystic Kidney Disease, Polymyalgia Rhuematica, Polymyositis, Porphyria, Post Herniorrhaphy Pain Syndrome, Post Mastectomy, Postoperative Pain, Pain Syndrome, Post Stroke Pain, Post Thoracotomy Pain Syndrome, Postherpetic Neuralgia (Shingles), Post-Polio Syndrome, Primary Lateral Sclerosis, Psoriatic Arthritis, Pudendal Neuralgia, Radiculopathy, Raynaud's Disease, Rheumatoid Arthritis (RA), Sacroiliac Joint Dysfunction, Sarcoidosi, Scheuemann's Kyphosis Disease, Sciatica, Scoliosis, Shingles (Herpes Zoster), Sjogren's Syndrome, Spasmodic Torticollis, Sphincter of Oddi Dysfunction, Spinal Cerebellum Ataxia (SCA Ataxia), Spinal Cord Injury, Spinal Stenosis, Syringomyelia, Tarlov Cysts, Transverse Myelitis, Trigeminal Neuralgia, Neuropathic Pain, Ulcerative Colitis, Vascular Pain or Vulvodynia.

379. The method of claim 377, wherein the itch associated condition is chronic itch; contact dermatitis; Allergic blepharitis; Anemia; Atopic dermatitis; Bullous pemphigoid; Candidiasis; Chicken pox; end-stage renal failure; hemodialysis; Chronic urticaria; Contact dermatitis, Atopic Dermatitis; Dermatitis herpetiformis; Diabetes; Drug allergy, Dry Eye Syndrome, Dry skin; Dyshidrotic dermatitis; Ectopic eczema; Eosinophilic fasciitis; Epidermolysis bullosa; Erythrasma; Food allergy; Folliculitis; Fungal skin infection; Hemorrhoids; Herpes; HIV infection; Hodgkin's disease; Hyperthyroidism; Iodinated contrast dye allergy; Iron deficiency anemia; Kidney disease; Leukemia, porphyrias; Lymphoma; Malignancy; Mastocystosis; Multiple myeloma; Neurodermatitis; Occular Itch, Onchocerciasis; Paget's disease; Pediculosis; Polycythemia rubra vera; Prurigo nodularis; Lichen Planus; Lichen Sclerosis; Pruritus ani; Pseudorabies; Psoriasis; Rectal prolapse; Sarcoidosis granulomas; Scabies; Schistosomiasis; Scleroderma, Severe stress, Stasia dermatitis; Swimmer's itch; Thyroid disease; Tinea cruris; Rosacea; Cutaneous amyloidosis; Scleroderma; Acne; wound healing; burn healing; ocular itch; or Urticaria.

380. The method of claim 377, wherein the itch associated condition is urticaria, pruritus, atopic dermatitis, dry skin, psoriasis, contact dermatitis, or eczema.

381. The method of claim 377, wherein the ocular associated condition is dry eye syndrome/keratoconjunctivitis sicca and related conditions, including xeropthalmia, meibomian gland dysfunction and lacrimal gland dysfunction; dry eye associated with other medical conditions including dacryoadenitis, dacryocystitis, allergic conjunctivitis, blepharitis, rheumatoid arthritis, systemic lupus erythematous, scleroderma, Sjogren's syndrome, Stevens-Johnson syndrome, sarcoidosis, sympathetic opthalmia, diabetic retinopathy, parasitic eye infections, thyroid disorders, and vitamin A deficiency; dry eye associated with medications such as antihistamines, decongestants, anti-depressants, tranquilizers, diuretics, hormone replacement, oral contraceptives, antihypertensives, isotretonin treatments for acne, and anticholinergic drugs; and dry eye associated with eye surgery including laser eye surgery, glaucoma surgery, corneal transplantation, and cataract removal surgery.

382. The method of claim 377, wherein the cardiovascular and renal disease associate condition peripheral vascular disease, cerebrovascular disease, coronary artery disease, cardiac hypertrophy, cardiac fibrosis, cardiovascular hypertension, renovascular hypertension, renal fibrosis, renal disease, nephritis, atherosclerosis, coronary atherosclerotic heart disease, acute myocardial infarction, stroke, thrombosis, coronary atherothrombosis, pulmonary embolism, myocardial ischemia, carotid stenosis, vertebral stenosis, intracranial stenosis, and aneurysms as well as treatment of cardiac dysfunction induced by sepsis, rheumatic fever, or other acute or chronic disorders that influence cardiovascular and renal function such as diabetes.

383. The method of claim 377, wherein the inflammatory or autoimmune disorder is chronic inflammation, mast cell activation syndrome, Multiple Sclerosis, Steven Johnson's Syndrome, Toxic Epidermal Necrolysis, appendicitis, bursitis, cutaneous lupus, colitis, cystitis, dermatitis, phlebitis, reflex sympathetic dystrophy/complex regional pain syndrome (rsd/crps), rhinitis, tendonitis, tonsillitis, acne vulgaris, sinusitis, rosacea, psoriasis, graft-versus-host disease, reactive airway disorder, asthma, airway infection, autoinflammatory disease, celiac disease, chronic prostatitis, diverticulitis, glomerulonephritis, hidradenitis suppurativa, hypersensitivities, intestinal disorder, epithelial intestinal disorder, inflammatory bowel disease, irritable bowel syndrome, Crohn's Disease, ulcerative colitis, lupus erythematous, interstitial cystitis, otitis, pelvic inflammatory disease, endometrial pain, reperfusion injury, rheumatic fever, rheumatoid arthritis, sarcoidosis, transplant rejection, psoriasis, lung inflammation, chronic obstructive pulmonary disease, cardiovascular disease, or vasculitis.

384. The method of claim 377, wherein the cognitive impairment associated condition includes neurodegenerative diseases including Parkinson's disease, Alzheimer's disease, Huntington's disease, Lewy body dementia, frontotemporal dementia, progressive supranuclear palsy, corticobasal syndrome, frontotemporal lobar degeneration, amyotrophic lateral sclerosis and multiple sclerosis, as well as age-induced cognitive impairment, vascular cognitive impairment and post-stroke cognitive impairment.

385. The method of claim 377, wherein the cancer related condition is acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

386. The method of claim 377, wherein the cancer related condition is lung cancer, pancreatic cancer, or skin cancer.

387. The method of claim 377, wherein the cancer related condition is lung cancer.

388. The method of claim 377, wherein the cancer related condition is pancreatic cancer.

389. The method of claim 377, wherein the cancer related condition is skin cancer.

390. The method of claim 389, wherein the skin cancer related condition is melanoma.
